(19) United States
(12) Patent Application Publication
(10) Pub. No.: US 2022/0259226 A1
(43) Pub. Date: Aug. 18, 2022

(54) MACROCYCLIC MCL-1 INHIBITORS AND METHODS OF USE

(71) Applicants: AbbVie Inc., North Chicago, IL (US); AbbVie Deutschland GmbH & Co. KG, Wiesbaden (DE)

(72) Inventors: Wilfried Braje, Wiesbaden (DE); George Doherty, Libertyville, IL (US); Katja Jantos, Wiesbaden (DE); Cheng Ji, Buffalo Grove, IL (US); Andrew Judd, Grayslake, IL (US); Aaron Kunzer, Arlington Heights, IL (US); Anthony Mastracchio, Libertyville, IL (US); Xiaohong Song, Grayslake, IL (US); Andrew Souers, Libertyville, IL (US); Gerard Sullivan, Libertyville, IL (US); Zhi-Fu Tao, Vernon Hills, IL (US); Chunqiu Lai, Libertyville, IL (US); Jesse Teske, Lake Bluff, IL (US); Michael Wendt, Vernon Hills, IL (US); Patrick Brady, Grayslake, IL (US); Xilu Wang, Libertyville, IL (US); Thomas Penning, Elmhurst, IL (US); Yujia Dai, Gurnee, IL (US); Jane Gong, Deerfield, IL (US); Roberto Risi, Pleasant Prairie, WI (US); Yiyun Yu, Lake Bluff, IL (US); Guidong Zhu, Gurnee, IL (US)

(21) Appl. No.: 17/660,355

(22) Filed: Apr. 22, 2022

Related U.S. Application Data
(63) Continuation of application No. 17/079,141, filed on Oct. 23, 2020, now abandoned.
Application No. 17/079,141 is a division of application No. 16/575,114, filed on Sep. 18, 2019, now abandoned.
Application No. 16/575,114 is a division of application No. 15/998,688, filed on Aug. 15, 2018, now abandoned.
(60) Provisional application No. 62/555,470, filed on Sep. 7, 2017.
(60) Provisional application No. 62/545,836, filed on Aug. 15, 2017.

(51) Int. Cl. C07D 498/18; A61P 35/00; C07D 519/00; C07D 495/18; C07D 495/16; A61K 31/519; C07D 491/16; C07D 498/16

(52) U.S. Cl. CPC C07D 498/18; A61P 35/00; C07D 519/00; C07D 498/16; C07D 495/16; A61K 31/519; C07D 491/16; C07D 495/18

(57) ABSTRACT

The present disclosure provides for compounds of Formula (I)

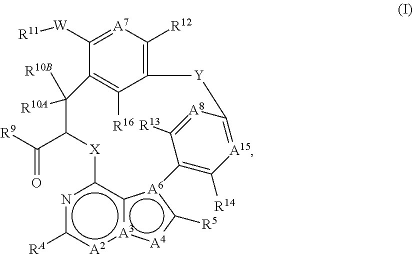

wherein A², A³, A⁴, A⁶, A⁷, A⁸, A¹⁵, R^{A}, R⁵, R⁹, R^{10A}, R^{10B}, R¹¹, R¹², R¹³, R¹⁴, R¹⁶, W, X, and Y have any of the values defined in the specification, and pharmaceutically acceptable salts thereof, that are useful as agents for the treatment of diseases and conditions, including cancer. Also provided are pharmaceutical compositions comprising compounds of Formula (I).

### BACKGROUND

### Technical Field

This disclosure relates to inhibitors of induced myeloid leukemia cell differentiation protein (MCL-1), compositions containing compounds described herein, and methods of treatment thereof.

### Description of Related Technology

Apoptosis, a type of programmed cell death, is critical for normal development and for preservation of cellular homeostasis. Dysregulation of apoptosis is recognized to play an important role in the development of various diseases. For example, blocks in apoptotic signaling are a common requirement for oncogenesis, tumor maintenance and chemoresistance (Hanahan, D. et al. Cell 2000, 100, 57). Apoptotic pathways can be divided into two categories, intrinsic and extrinsic, depending on the origin of the death signal. The intrinsic pathway, or mitochondrial apoptotic pathway, is initiated by intracellular signals that ultimately lead to mitochondrial outer membrane permeabilization (MOMP), caspase activation and cell death.

The intrinsic mitochondrial apoptotic pathway is highly regulated, and the dynamic binding interactions between the pro-apoptotic (e.g. BAX, BAK, BAD, BIM, NOXA) and anti-apoptotic (e.g. BCL-2, BCL-XL, MCL-1) BCL-2 family members control commitment to cell death (Youle, R. J. et al. Nat. Rev. Mol. Cell Biol. 2008, 9, 47). BAK and BAX are essential mediators that upon conformational activation cause MOMP, an irreversible event that subsequently leads to cytochrome c release, caspase activation and cell death. Anti-apoptotic BCL-2 family members such as BCL-2, BCL-XL and MCL-1 can bind and sequester their pro-apoptotic counterparts, thus preventing BAX/BAK activation and promoting cell survival.

BCL-2 plays a dominant role in the survival of several hematological malignancies where it is frequently overexpressed, whereas BCL-XL is a key survival protein in some hematological and solid tumors. The related anti-apoptotic protein MCL-1 is implicated in mediating malignant cell survival in a number of primary tumor types (Ashkenazi, A. et al. Nature Rev Drug Discovery 2017, 16, 273). MCL-1 gene amplifications are frequently found in human cancers, including breast cancer and non-small cell lung cancer (Beroukhim, R. et al. Nature 2010, 463, 899), and the MCL-1 protein has been shown to mediate survival in models of multiple myeloma (Derenn, S. et al. Blood 2002, 100, 194), acute myeloid leukemia (Glaser, S. et al. Genes Dev 2012, 26, 120) and MYC-driven lymphomas (Kelly, G. et al. Genes Dev 2014, 28, 58). Specific compounds that broadly inhibit gene transcription (e.g., CDK9 inhibitors) exert their cytotoxic effects on tumor cells, at least in part, by down-regulating MCL-1 (Kotschy, A. et al. Nature 2016, 538, 477); alvocidib (Kim, W. et al. Blood 2015, 126, 1343) and dinaciclib (Gregory, G. et al. Leukemia 2015, 29, 1437) are two examples that have demonstrated clinical proof-of-concept in patients with hematological malignancies. Literature data supports a role for MCL-1 as a resistance factor to anticancer therapies such gemcitabine, vincristine and taxol (Wertz, I. E. et al. Nature 2011, 471, 110). Accordingly, there is a need in the therapeutic arts for compounds which inhibit the activity of the MCL-1 protein.

### SUMMARY

In embodiments the present disclosure provides for compounds of Formula (I) or a pharmaceutically acceptable salt thereof,

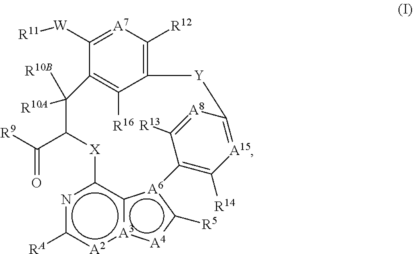

wherein
A² is CR², A³ is N, A⁴ is CR^{4a}, and A⁶ is C; or
A² is CR², A³ is N, A⁴ is O or S, and A⁶ is C; or
A² is N, A³ is C, A⁴ is O or S and A⁶ is C; or
A² is N, A³ is C, A⁴ is CR^{4a}, and A⁶ is N;
R^{A} is hydrogen, CH₃, halogen, CN, CH₂F, CHF₂, or CF₃;
X is O, or N(R^{x2}); wherein R^{x2} is hydrogen, C₁-C₃ alkyl, or unsubstituted cyclopropyl;
Y is (CH₂)ₘ, —CH═CH—(CH₂)ₙ—, —(CH₂)ₚ—CH═CH—, or —(CH₂)_{q}—CH═CH—(CH₂)ᵣ—; wherein 0, 1, 2, or 3 CH₂ groups are each independently replaced by O, N(R^{ya}), C(R^{ya})(R^{yb}), C(O), NC(O)R^{ya}, or S(O)₂;
m is 2, 3, 4, or 5;
n is 1, 2, or 3;
p is 1, 2, or 3;
q is 1 or 2; and
r is 1 or 2; wherein the sum of q and r is 2 or 3;
R^{ya}, at each occurrence, is independently hydrogen, C₂-C₆ alkenyl, C₂-C₆ alkynyl, C₁-C₆ alkyl, or C₁-C₆ haloalkyl; wherein the C₂-C₆ alkenyl, C₂-C₆ alkynyl, C₁-C₆ alkyl, and C₁-C₆ haloalkyl are optionally substituted with 1 or 2 substituents independently selected from the group consisting of oxo, —N(R^{yd})(R^{ye}), G¹, —OR^{yf}, —SR^{yg}, —S(O)₂N(R^{yd})(R^{ye}), and —S(O)₂-G¹; and
R^{yb} is C₂-C₆ alkenyl, C₂-C₆ alkynyl, G¹, C₁-C₆ alkyl, or C₁-C₆ haloalkyl; wherein the C₂-C₆ alkenyl, C₂-C₆ alkynyl, C₁-C₆ alkyl, and C₁-C₆ haloalkyl are optionally substituted with 1 or 2 substituents independently selected from the group consisting of oxo, —N(R^{yd})(R^{ye}), G¹, —OR^{yf}, —SR^{yg}, —S(O)₂N(R^{yd})(R^{ye}), and —S(O)₂-G¹; or
R^{ya} and R^{yb}, together with the carbon atom to which they are attached, form a C₃-C₇ monocyclic cycloalkyl, C₄-C₇ monocyclic cycloalkenyl, or a 4-7 membered monocyclic heterocycle; wherein the C₃-C₇ monocyclic cycloalkyl, C₄-C₇ monocyclic cycloalkenyl, and the 4-7 membered monocyclic heterocycle are each optionally substituted with 1 —OR^{m} and 0, 1, 2, or 3 independently selected R^{s} groups;
R^{yd}, R^{ye}, R^{yf}, and R^{yg}, at each occurrence, are each independently hydrogen, G¹, C₁-C₆ alkyl, or C₁-C₆ haloalkyl; wherein the C₁-C₆ alkyl and the C₁-C₆ haloalkyl are optionally substituted with one substituent selected from the group consisting of G¹, —OR^{th}, —SR^{yh}, —SO₂R^{yh}, and —N(R^{yi})(R^{yk});
G¹, at each occurrence, is piperazinyl, piperidinyl, pyrrolidinyl, thiomorpholinyl, tetrahydropyranyl, morpholinyl, or oxetanyl; wherein each G¹ is optionally substituted with 1 —OR^{m} and 0, 1, 2, or 3 substituents independently selected from the group consisting of G², —(C₁-C₆ alkylenyl)-G², and R^{s};
G², at each occurrence, is a C₃-C₇ monocyclic cycloalkyl, C₄-C₇ monocyclic cycloalkenyl, oxetanyl, or morpholinyl; wherein each G² is optionally substituted with 1 independently selected R^{t} groups;
R² is independently hydrogen, halogen, CH₃, or CN;
R^{4a}, at each occurrence, is independently hydrogen, halogen, CN, C₂-C₄ alkenyl, C₂-C₄ alkynyl, C₁-C₄ alkyl, C₁-C₄ haloalkyl, G^{A}, C₁-C₄ alkyl-G^{A}, or C₁-C₄ alkyl-O-G^{A}; wherein each G^{A} is independently C₆-C₁₀ aryl, C₃-C₇ monocyclic cycloalkyl, C₄-C₇ monocyclic cycloalkenyl, or 4-7 membered heterocycle; wherein each G^{A} is optionally substituted with 1, 2, or 3 R^{u} groups;
R⁵ is independently hydrogen, halogen, G³, C₁-C₆ alkyl, C₂-C₆ alkenyl, or C₂-C₆ alkynyl; wherein the C₁-C₆ alkyl, C₂-C₆ alkenyl, and C₂-C₆ alkynyl are each optionally substituted with one G³;
G³, at each occurrence, is independently C₆-C₁₀ aryl, 5-11 membered heteroaryl, C₃-C₁₁ cycloalkyl, cycloalkenyl, oxetanyl, or 2-oxaspiro[3.3]heptanyl; wherein each G³ is optionally substituted with 1, 2, or 3 R^{v} groups;
A⁷ is N or CR⁷;
A⁸ is N or CR⁸;
A¹⁵ is N or CR¹⁵;
R⁷, R¹² and R⁶ are each independently hydrogen, halogen, C₁-C₄ alkyl, C₁-C₄ haloalkyl, —CN, —OR^{7a}, —SR^{7a}, or —N(R^{7b})(R^{7c});
R⁸, R¹³, R¹⁴, and R¹⁵, are each independently hydrogen, halogen, C₁-C₄ alkyl, C₁-C₄ haloalkyl, —CN, —OR^{8a}, —SR^{8a}, —N(R^{8b})(R^{8c}), or C₃-C₄ monocyclic cycloalkyl; wherein the C₃-C₄ monocyclic cycloalkyl is optionally substituted with one or two substituents independently selected from the group consisting of halogen, C₁-C₃ alkyl, and C₁-C₃ haloalkyl; or
R⁸ and R¹³ are each independently hydrogen, halogen, C₁-C₄ alkyl, C₁-C₄ haloalkyl, —CN, —OR^{8a}, —SR^{8a}, —N(R^{8b})(R^{8c}), or C₃-C₄ monocyclic cycloalkyl; wherein the C₃-C₄ monocyclic cycloalkyl is optionally substituted with one or two substituents independently selected from the group consisting of halogen, C₁-C₃ alkyl, and C₁-C₃ haloalkyl; and
R¹⁴ and R¹⁵, together with the carbon atoms to which they are attached, form a monocyclic ring selected from the group consisting of benzene, cyclobutane, cyclopentane, and pyridine; wherein the monocyclic ring is optionally substituted with 1, 2, or 3 substituents independently selected from the group consisting of halogen, C₁-C₄ alkyl, C₁-C₄ haloalkyl, —CN, —OR^{8a}, —SR^{8a}, and —N(R^{8b})(R^{8c});
R⁹ is —OH, —O—C₁-C₄ alkyl, —O—CH₂—OC(O)(C₁-C₆ alkyl), —NHOH,

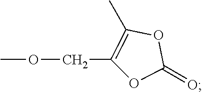

or —N(H)S(O)₂—(C₁-C₆ alkyl);
R^{10A} and R^{10B}, are each independently hydrogen, C₁-C₃ alkyl, or C₁-C₃ haloalkyl; or R^{10A} and R^{10B}, together with the carbon atom to which they are attached, form a cyclopropyl; wherein the cyclopropyl is optionally substituted with one or two substituents independently selected from the group consisting of halogen and CH₃;
W is —CH═CH—, C₁-C₄ alkyl, —O—CHF—, -L¹-CH₂—, or —CH₂-L¹-; wherein L¹ at each occurrence, is independently O, S, S(O), S(O)₂, S(O)₂N(H), N(H), or N(C₁-C₃ alkyl);
R¹¹ is a C₆-C₁₀ aryl or a 5-11 membered heteroaryl; wherein each R¹¹ is optionally substituted with 1, 2, or 3 independently selected R^{w} groups;
R^{w}, at each occurrence, is independently C₁-C₆ alkyl, C₂-C₆ alkenyl, C₂-C₆ alkynyl, halogen, C₁-C₆ haloalkyl, —CN, NO₂, —OR^{11a}, —SR^{11b}, —S(O)₂R^{11b}, —S(O)₂N(R^{11c})₂, —C(O)R^{11a}, —C(O)N(R^{11c})₂, —N(R^{11c})₂, —N(R^{11c})C(O)R^{11b}, —N(R^{11c})S(O)₂R^{11b}, —N(R^{11c})C(O)O(R^{11b}), —N(R^{11c})C(O)N(R^{11c})₂, G⁴, —(C₁-C₆ alkylenyl)-OR^{11a}, —(C₁-C₆alkylenyl)-OC(O)N(R^{11c})₂, —(C₁-C₆ alkylenyl)-SR^{11a}, —(C₁-C₆ alkylenyl)-S(O)₂R^{11b}, —(C₁-C₆ alkylenyl)-S(O)₂N(R^{11c})₂, —(C₁-C₆ alkylenyl)-C(O)R^{11a}, —(C₁-C₆ alkylenyl)-C(O)N(R^{11c})₂, —(C₁-C₆ alkylenyl)-N(R^{11c})₂, —(C₁-C₆ alkylenyl)-N(R^{11c})C(O)R^{11b}, —(C₁-C₆alkylenyl)-N(R^{11c})S(O)₂R^{11b}, —(C₁-C₆ alkylenyl)-N(R^{11c})C(O)O(R^{11b}), —(C₁-C₆alkylenyl)-N(R^{11c})C(O)N(R^{11c})₂, —(C₁-C₆ alkylenyl)-CN, or —(C₁-C₆ alkylenyl)-G⁴;
R^{11a} and R^{11c}, at each occurrence, are each independently hydrogen, C₁-C₆ alkyl, C₂-C₆ alkenyl, C₁-C₆ haloalkyl, G⁴, —(C₂-C₆alkylenyl)-OR^{11d}, —(C₂-C₆ alkylenyl)-N(R^{11e})₂, or —(C₂-C₆ alkylenyl)-G⁴;
R^{11b}, at each occurrence, is independently C₁-C₆ alkyl, C₂-C₆ alkenyl, C₁-C₆ haloalkyl, G⁴, —(C₂-C₆ alkylenyl)-OR^{11d}, —(C₂-C₆ alkylenyl)-N(R^{11e})₂, or —(C₂-C₆ alkylenyl)-G⁴;
G⁴, at each occurrence, is independently phenyl, monocyclic heteroaryl, C₃-C₁₁ cycloalkyl, C₄-C₁₁ cycloalkenyl, oxetanyl, tetrahydrofuranyl, tetrahydropyranyl, morpholinyl, 2,6-dioxa-9-azaspiro[4.5]decanyl, 2-oxa-5-azabicyclo[2.2.1]heptanyl, 3-oxa-8-azabicyclo[3.2.1]octanyl, piperidinyl, azetidinyl, dihydropyranyl, tetrahydropyridinyl, dihydropyrrolyl, or pyrrolidinyl; wherein each G⁴ is optionally substituted with 1 —OR^{m} and 0, 1, 2, 3, or 4 substituents independently selected from the group consisting of G⁵, R^{y}, —(C₁-C₆ alkylenyl)-G⁵, and -L²-(C₁-C₆ alkylenyl), -G⁵;
L² is O, C(O), N(H), N(C₁-C₆ alkyl), NHC(O), C(O)O, S, S(O), or S(O)₂;
s is 0 or 1;
G⁵, at each occurrence, is independently phenyl, monocyclic heteroaryl, C₃-C₇ monocyclic cycloalkyl, C₄-C₇ monocyclic cycloalkenyl, or piperazine; wherein each G⁵ is optionally substituted with 1 independently selected —OR^{m} or R^{z} group;
R^{s}, R^{t}, R^{u}, R^{v}, R^{y}, and R^{z}, at each occurrence, are each independently C₁-C₆ alkyl, C₂-C₆ alkenyl, C₂-C₆ alkynyl, halogen, C₁-C₆ haloalkyl, —CN, oxo, NO₂, P(O)(R^{k})₂, —OC(O)R^{k}, —OC(O)N(R^{j})₂, —SR^{j}, —S(O)₂R^{k}, —S(O)₂N(R^{j})₂, —C(O)R^{j}, —C(O)N(R^{j})₂, —N(R^{j})₂, —N(R^{j})C(O)R^{k}, —N(R^{j})S(O)₂R^{k}, —N(R^{j})C(O)O(R^{k}), —N(R^{j})C(O)N(R^{j})₂, —(C₁-C₆ alkylenyl)-OR^{j}, —(C₁-C₆ alkylenyl)-OC(O)N(R^{j})₂, —(C₁-C₆alkylenyl)-SR^{j}, —(C₁-C₆alkylenyl)-S(O)₂R^{k}, —(C₁-C₆ alkylenyl)-S(O)₂N(R^{j})₂, —(C₁-C₆ alkylenyl)-C(O)R^{j}, —(C₁-C₆ alkylenyl)-C(O)N(R^{j})₂, —(C₁-C₆ alkylenyl)-N(R^{j})₂, —(C₁-C₆alkylenyl)-N(R^{j})C(O)R^{k}, —(C₁-C₆ alkylenyl)-N(R^{j})S(O)₂R^{k}, —(C₁-C₆ alkylenyl)-N(R^{j})C(O)O(R^{k}), —(C₁-C₆ alkylenyl)-N(R^{j})C(O)N(R^{j})₂, or —(C₁-C₆ alkylenyl)-CN;
R^{m} is hydrogen, C₁-C₆ alkyl, C₁-C₆ haloalkyl, —(C₂-C₆alkylenyl)-OR^{j}, or —(C₂-C₆ alkylenyl)-N(R^{j})₂;
R^{yh}, R^{yi}, R^{yk}, R^{7a}, R^{7b}, R^{7c}, R^{8a}, R^{8b}, R^{8c}, R^{11d}, R^{11e}, and R^{j}, at each occurrence, are each independently hydrogen, C₁-C₆ alkyl, or C₁-C₆ haloalkyl; and
R^{k}, at each occurrence, is independently C₁-C₆ alkyl or C₁-C₆ haloalkyl.

In embodiments, the present disclosure provides for methods of treating or preventing disorders that are amenable to inhibition of MCL-1. Such methods comprise administering to the subject a therapeutically effective amount of a compound of Formula (I), alone, or in combination with a pharmaceutically acceptable carrier.

In embodiments, some of the methods are directed to treating or preventing cancer. That is, in embodiments, the present disclosure provides for methods for treating or preventing cancer, wherein such methods comprise administering to the subject a therapeutically effective amount of a compound of Formula (I), alone, or in combination with a pharmaceutically acceptable carrier.

In embodiments, the present disclosure relates to methods of treating cancer in a subject comprising administering a therapeutically effective amount of a compound of Formula (I), or a pharmaceutically acceptable salt thereof, to a subject in need thereof. In certain embodiments, the cancer is multiple myeloma. In certain embodiments, the methods further comprise administering a therapeutically effective amount of at least one additional therapeutic agent.

In embodiments, the present disclosure provides the use of a compound of Formula (I), alone or in combination with at least one additional therapeutic agent, in the manufacture of a medicament for treating or preventing conditions and disorders disclosed herein, with or without a pharmaceutically acceptable carrier.

Pharmaceutical compositions comprising a compound of Formula (I), or a pharmaceutically acceptable salt, alone or in combination with at least one additional therapeutic agent, are also provided.

### DETAILED DESCRIPTION

In embodiments, the present disclosure provides for compounds of Formula (I), or a pharmaceutically acceptable salt thereof,

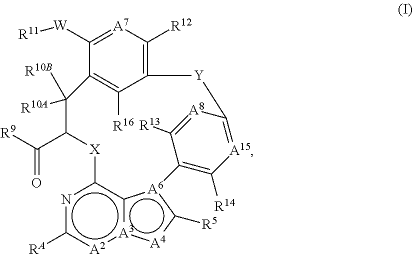

wherein A², A³, A⁴, A⁶, A⁷, A⁸, A¹⁵, R^{A}, R⁵, R⁹, R^{10A}, R^{10B}, R¹¹, R¹², R¹³, R¹⁴, R¹⁶, W, X, and Y are defined above in the Summary and below in the Detailed Description. Further, compositions comprising such compounds and methods for treating conditions and disorders using such compounds and compositions are also included.

Compounds included herein may contain one or more variable(s) that occur more than one time in any substituent or in the Formulae herein. Definition of a variable on each occurrence is independent of its definition at another occurrence. Further, combinations of substituents are permissible only if such combinations result in stable compounds. Stable compounds are compounds which can be isolated from a reaction mixture.

### Definitions

It is noted that, as used in this specification and the intended claims, the singular form “a,” “an,” and “the” include plural referents unless the context clearly dictates otherwise. Thus, for example, reference to “a compound” includes a single compound as well as one or more of the same or different compounds, reference to “a pharmaceutically acceptable carrier” means a single pharmaceutically acceptable carrier as well as one or more pharmaceutically acceptable carriers, and the like.

As used in the specification and the appended claims, unless specified to the contrary, the following terms have the meaning indicated:

The term “alkenyl” as used herein, means a straight or branched hydrocarbon chain containing from 2 to 10 carbons and containing at least one carbon-carbon double bond. The term “C₂-C₆ alkenyl” and “C₂-C₄ alkenyl” means an alkenyl group containing 2-6 carbon atoms and 2-4 carbon atoms respectively. Non-limiting examples of alkenyl include buta-1,3-dienyl, ethenyl, 2-propenyl, 2-methyl-2-propenyl, 3-butenyl, 4-pentenyl, and 5-hexenyl. The terms “alkenyl,” “C₂-C₆ alkenyl,” and “C₂-C₄ alkenyl” used herein are unsubstituted, unless otherwise indicated.

The term “alkyl” as used herein, means a saturated, straight or branched hydrocarbon chain radical. In some instances, the number of carbon atoms in an alkyl moiety is indicated by the prefix “Cₓ-C_{y}”, wherein x is the minimum and y is the maximum number of carbon atoms in the substituent. Thus, for example, “C₁-C₆ alkyl” means an alkyl substituent containing from 1 to 6 carbon atoms, “C₁-C₄ alkyl” means an alkyl substituent containing from 1 to 4 carbon atoms, and “C₁-C₃ alkyl” means an alkyl substituent containing from 1 to 3 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 1-methylbutyl, 2-methylbutyl, 3-methylbutyl, 3,3-dimethylbutyl, 1,1-dimethylpropyl, 1,2-dimethylpropyl, 2,2-dimethylpropyl, 1-methylpropyl, 2-methylpropyl, 1-ethylpropyl, and 1,2,2-trimethylpropyl. The terms “alkyl,” “C₁-C₆ alkyl,” “C₁-C₄ alkyl,” and “C₁-C₃ alkyl” used herein are unsubstituted, unless otherwise indicated.

The term “alkylene” or “alkylenyl” means a divalent radical derived from a straight or branched, saturated hydrocarbon chain, for example, of 1 to 10 carbon atoms or of 1 to 6 carbon atoms (C₁-C₆ alkylenyl) or of 1 to 4 carbon atoms (C₁-C₄ alkylenyl) or of 1 to 3 carbon atoms (C₁-C₃ alkylenyl) or of 2 to 6 carbon atoms (C₂-C₆ alkylenyl). Examples of alkylenyl include, but are not limited to, —CH₂—, —CH₂CH₂—, —C((CH₃)₂)—CH₂CH₂CH₂—, —C((CH₃)₂)—CH₂CH₂, —CH₂CH₂CH₂CH₂—, and —CH₂CH(CH₃)CH₂—.

The term “C₂-C₆ alkynyl” and “C₂-C₄ alkynyl” as used herein, means a straight or branched chain hydrocarbon radical containing from 2 to 6 carbon atoms and 2 to 4 carbon atoms respectively, and containing at least one carbon-carbon triple bond. Representative examples of C₂-C₆ alkynyl and C₂-C₄ alkynyl include, but are not limited, to acetylenyl, 1-propynyl, 2-propynyl, 3-butynyl, 2-pentynyl, and 1-butynyl. The terms “alkynyl,” “C₂-C₆ alkynyl,” and “C₂-C₄ alkynyl” used herein are unsubstituted, unless otherwise indicated.

The term “C₆-C₁₀ aryl” as used herein, means phenyl or a bicyclic aryl. The bicyclic aryl is naphthyl, or a phenyl fused to a C₃-C₆ monocyclic cycloalkyl, or a phenyl fused to a C₄-C₆ monocyclic cycloalkenyl. Non-limiting examples of the aryl groups include dihydroindenyl, indenyl, naphthyl, dihydronaphthalenyl, and tetrahydronaphthalenyl.

The term “C₃-C₁₁cycloalkyl” as used herein, means a hydrocarbon ring radical containing 3-11 carbon atoms, zero heteroatom, and zero double bonds. The C₃-C₁₁ cycloalkyl group may be a single-ring (monocyclic) or have two or more rings (polycyclic or bicyclic). Monocyclic cycloalkyl groups typically contain from 3 to 8 carbon ring atoms (C₃-C₈ monocyclic cycloalkyl) or 3 to 7 carbon ring atoms (C₃-C₇ monocyclic cycloalkyl), and even more typically 3-6 carbon ring atoms (C₃-C₆ monocyclic cycloalkyl). Examples of monocyclic cycloalkyls include cyclopropyl, cyclobutyl, cyclopentyl, cyclohexyl, cycloheptyl, and cyclooctyl. Polycyclic cycloalkyl groups contain two or more rings, and bicyclic cycloalkyls contain two rings. In certain embodiments, the polycyclic cycloalkyl groups contain 2 or 3 rings. The rings within the polycyclic and the bicyclic cycloalkyl groups may be in a bridged, fused, or spiro orientation, or combinations thereof. In a spirocyclic cycloalkyl, one atom is common to two different rings. An example of a spirocyclic cycloalkyl is spiro[4.5]decane. In a bridged cycloalkyl, the rings share at least two non-adjacent atoms. Examples of bridged cycloalkyls include, but are not limited to, bicyclo[1.1.1]pentanyl, bicyclo[2.2.2]octanyl, bicyclo[3.2.1]octanyl, bicyclo[3.1.1]heptyl, bicyclo[2.2.1]heptyl, bicyclo[3.2.2]nonyl, bicyclo[3.3.1]nonyl, bicyclo[4.2.1]nonyl, tricyclo[3.3.1.0^{3,7}]nonyl (octahydro-2,5-methanopentalenyl or noradamantyl), tricyclo[3.3.1.1^{3,7}]decyl (adamantyl), and tricyclo[4.3.1.1^{3,8}]undecyl (homoadamantyl). In a fused ring cycloalkyl, the rings share one common bond. Example of fused-ring cycloalkyls include, but not limited to, decalin (decahydronaphthyl).

The term “C₃-C₇ monocyclic cycloalkyl” as used herein, means cyclopropyl, cyclobutyl, cyclopentyl, cyclohexyl, and cycloheptyl.

The term “C₄-C₁₁cycloalkenyl” as used herein, refers to a monocyclic or a bicyclic hydrocarbon ring radical. The monocyclic cycloalkenyl has four-, five-, six-, seven- or eight carbon atoms and zero heteroatoms. The four-membered ring systems have one double bond, the five- or six-membered ring systems have one or two double bonds, and the seven- or eight-membered ring systems have one, two, or three double bonds. Representative examples of monocyclic cycloalkenyl groups include, but are not limited to, cyclobutenyl, cyclopentenyl, cyclohexenyl, cycloheptenyl, and cyclooctenyl. The bicyclic cycloalkenyl is a monocyclic cycloalkenyl fused to a monocyclic cycloalkyl group, or a monocyclic cycloalkenyl fused to a monocyclic cycloalkenyl group. The monocyclic and bicyclic cycloalkenyl ring may contain one or two alkylene bridges, each consisting of one, two, or three carbon atoms, and each linking two non-adjacent carbon atoms of the ring system. Representative examples of the bicyclic cycloalkenyl groups include, but are not limited to, 4,5,6,7-tetrahydro-3aH-indene, octahydronaphthalenyl, and 1,6-dihydro-pentalene. The monocyclic and the bicyclic cycloalkenyls, including exemplary rings, are optionally substituted unless otherwise indicated. The monocyclic cycloalkenyl and bicyclic cycloalkenyl are attached to the parent molecular moiety through any substitutable atom contained within the ring systems.

The term “C₃-C₆ monocyclic cycloalkyl” as used herein, means cyclopropyl, cyclobutyl, cyclopentyl, and cyclohexyl.

The term “C₃-C₄ monocyclic cycloalkyl” as used herein, means cyclopropyl and cyclobutyl.

The term “C₄-C₆ monocyclic cycloalkenyl” as used herein, means cyclobutenyl, cyclopentenyl, and cyclohexenyl.

The term “halo” or “halogen” as used herein, means Cl, Br, I, and F.

The term “haloalkyl” as used herein, means an alkyl group, as defined herein, in which one, two, three, four, five, or six hydrogen atoms are replaced by halogen. The term “C₁-C₆ haloalkyl” means a C₁-C₆ alkyl group, as defined herein, in which one, two, three, four, five, or six hydrogen atoms are replaced by halogen. The term “C₁-C₄ haloalkyl” means a C₁-C₄ alkyl group, as defined herein, in which one, two, three, four, or five hydrogen atoms are replaced by halogen. The term “C₁-C₃ haloalkyl” means a C₁-C₃ alkyl group, as defined herein, in which one, two, three, four, or five hydrogen atoms are replaced by halogen. Representative examples of haloalkyl include, but are not limited to, chloromethyl, 2-fluoroethyl, 2,2-difluoroethyl, fluoromethyl, 2,2,2-trifluoroethyl, trifluoromethyl, difluoromethyl, pentafluoroethyl, 2-chloro-3-fluoropentyl, trifluorobutyl, and trifluoropropyl. The terms “haloalkyl,” “C₁-C₆ haloalkyl,” “C₁-C₄ haloalkyl,” and “C₁-C₃ haloalkyl,” as used herein are unsubstituted, unless otherwise indicated.

The term “5-11 membered heteroaryl” as used herein, means a monocyclic heteroaryl and a bicyclic heteroaryl. The monocyclic heteroaryl is a five- or six-membered hydrocarbon ring wherein at least one carbon ring atom is replaced by heteroatom independently selected from the group consisting of O, N, and S. The five-membered ring contains two double bonds. The five membered ring may have one heteroatom selected from O or S; or one, two, three, or four nitrogen atoms and optionally one oxygen or one sulfur atom. The six-membered ring contains three double bonds and one, two, three or four nitrogen atoms. Examples of monocyclic heteroaryl include, but are not limited to, furanyl, imidazolyl, isoxazolyl, isothiazolyl, oxadiazolyl, 1,3-oxazolyl, pyridinyl, pyridazinyl, pyrimidinyl, pyrazinyl, pyrazolyl, pyrrolyl, tetrazolyl, thiadiazolyl, 1,3-thiazolyl, thienyl, triazolyl, and triazinyl. The bicyclic heteroaryl consists of a monocyclic heteroaryl fused to a phenyl, or a monocyclic heteroaryl fused to a monocyclic C₃-C₆ cycloalkyl, or a monocyclic heteroaryl fused to C₄-C₆ monocyclic cycloalkenyl, or a monocyclic heteroaryl fused to a monocyclic heteroaryl, or a monocyclic heteroaryl fused to a 4-7 membered monocyclic heterocycle. Representative examples of bicyclic heteroaryl groups include, but are not limited to, benzofuranyl, benzothienyl, benzoxazolyl, benzimidazolyl, benzoxadiazolyl, phthalazinyl, 2,6-dihydropyrrolo[3,4-c]pyrazol-5(4H)-yl, 6,7-dihydro-pyrazolo[1,5-a]pyrazin-5(4H)-yl, 6,7-dihydro-1,3-benzothiazolyl, imidazo[1,2-a]pyridinyl, indazolyl, indolyl, isoindolyl, isoquinolinyl, naphthyridinyl, pyridoimidazolyl, quinolinyl, 2,4,6,7-tetrahydro-5H-pyrazolo[4,3-c]pyridin-5-yl, thiazolo[5,4-b]pyridin-2-yl, thiazolo[5,4-d]pyrimidin-2-yl, and 5,6,7,8-tetrahydroquinolin-5-yl.

The term “4-11 membered heterocycle” as used herein, means a hydrocarbon ring radical of 4-11 carbon ring atoms wherein at least one carbon ring atom is replaced by atoms independently selected from the group consisting of O, N, S, P(═O), and Si. The 4-11 membered heterocycle ring may be a single ring (monocyclic) or have two or more rings (bicyclic or polycyclic). In certain embodiments, the monocyclic heterocycle is a four-, five-, six-, or seven-, membered hydrocarbon ring wherein at least one carbon ring atom is replaced by atoms independently selected from the group consisting of O, N, S, P(═O), and Si. In certain embodiments, the monocyclic heterocycle is a 4-6 membered hydrocarbon ring wherein at least one carbon ring atom is replaced by atoms independently selected from the group consisting of O, N, S, P(═O), and Si. A four-membered monocyclic heterocycle contains zero or one double bond, and one carbon ring atom replaced by an atom selected from the group consisting of O, N, and S. A five-membered monocyclic heterocycle contains zero or one double bond and one, two, or three carbon ring atoms replaced by atoms selected from the group consisting of O, N, S, P(═O), and Si. Examples of five-membered monocyclic heterocycles include those containing in the ring: 1 O; 1 S; 1 N; 1 P(═O); 1 Si; 2 N; 3 N; 1 S and 1 N; 1 S, and 2 N; 1 O and 1 N; or 1 O and 2 N. Non limiting examples of 5-membered monocyclic heterocyclic groups include 1,3-dioxolanyl, tetrahydrofuranyl, dihydrofuranyl, tetrahydrothienyl, dihydrothienyl, imidazolidinyl, oxazolidinyl, imidazolinyl, isoxazolidinyl, isothiazolidinyl, pyrazolidinyl, pyrazolinyl, pyrrolidinyl, 2-pyrrolinyl, 3-pyrrolinyl, thiazolinyl, and thiazolidinyl. A six-membered monocyclic heterocycle contains zero, one, or two double bonds and one, two, or three carbon ring atoms replaced by heteroatoms selected from the group consisting of O, N, S, P(═O), and Si. Examples of six-membered monocyclic heterocycles include those containing in the ring: 1 P(═O); 1 Si; 1 O; 2 O; 1 S; 2 S; 1 N; 2 N; 3 N; 1 S, 1 O, and 1 N; 1 S and 1 N; 1 S and 2 N; 1 S and 1 O; 1 S and 2 O; 1 O and 1 N; and 1 O and 2 N. Examples of six-membered monocyclic heterocycles include 1,3-oxazinanyl, tetrahydropyranyl, dihydropyranyl, 1,6-dihydropyridazinyl, 1,2-dihydropyrimidinyl, 1,6-dihydropyrimidinyl, dioxanyl, 1,4-dithianyl, hexahydropyrimidinyl, morpholinyl, piperazinyl, piperidinyl, 1,2,3,6-tetrahydropyridinyl, tetrahydrothiopyranyl, thiomorpholinyl, thioxanyl, and trithianyl Seven- and eight-membered monocyclic heterocycles contains zero, one, two, or three double bonds and one, two, or three carbon ring atoms replaced by heteroatoms selected from the group consisting of O, N, and S. Examples of monocyclic heterocycles include, but are not limited to, azetidinyl, azepanyl, aziridinyl, diazepanyl, 1,3-dioxanyl, 1,3-dioxolanyl, 1,3-dithiolanyl, 1,3-dithianyl, 1,6-dihydropyridazinyl, 1,2-dihydropyrimidinyl, 1,6-dihydropyrimidinyl, hexahydropyrimidinyl, imidazolinyl, imidazolidinyl, isoindolinyl, isothiazolinyl, isothiazolidinyl, isoxazolinyl, isoxazolidinyl, morpholinyl, oxadiazolinyl, oxadiazolidinyl, 1,3-oxazinanyl, oxazolinyl, 1,3-oxazolidinyl, oxetanyl, piperazinyl, piperidinyl, pyranyl, pyrazolinyl, pyrazolidinyl, pyrrolinyl, pyrrolidinyl, 1,2-dihydropyridinyl, tetrahydrofuranyl, tetrahydropyridinyl, tetrahydropyrimidinyl, tetrahydropyranyl, tetrahydrothienyl, thiadiazolinyl, thiadiazolidinyl, thiazolinyl, thiazolidinyl, thiomorpholinyl, thiopyranyl, and trithianyl Polycyclic heterocycle groups contain two or more rings, and bicyclic heterocycles contain two rings. In certain embodiments, the polycyclic heterocycle groups contain 2 or 3 rings. The rings within the polycyclic and the bicyclic heterocycle groups are in a bridged, fused, or spiro orientation, or combinations thereof. In a spirocyclic heterocycle, one atom is common to two different rings. Non limiting examples of spirocyclic heterocycles include 4,6-diazaspiro[2.4]heptanyl, 6-azaspiro[3.4]octane, 2-oxa-6-azaspiro[3.4]octan-6-yl, and 2,7-diazaspiro[4.4]nonane. In a fused ring heterocycle, the rings share one common bond. Examples of fused bicyclic heterocycles are a 4-6 membered monocyclic heterocycle fused to a phenyl group, or a 4-6 membered monocyclic heterocycle fused to a monocyclic C₃-C₆ cycloalkyl, or a 4-6 membered monocyclic heterocycle fused to a C₄-C₆ monocyclic cycloalkenyl, or a 4-6 membered monocyclic heterocycle fused to a 4-6 membered monocyclic heterocycle. Examples of fused bicyclic heterocycles include, but are not limited to hexahydropyrano[3,4-b][1,4]oxazin-1(5H)-yl, hexahydropyrrolo[3,4-c]pyrrol-2(1H)-yl, hexahydro-1H-imidazo[5,1-c][1,4]oxazinyl, hexahydro-1H-pyrrolo[1,2-c]imidazolyl, hexahydrocyclopenta[c]pyrrol-3a(1H)-yl, and 3-azabicyclo[3.1.0]hexanyl. In a bridged heterocycle, the rings share at least two non-adjacent atoms. Examples of such bridged heterocycles include, but are not limited to, azabicyclo[2.2.1]heptyl (including 2-azabicyclo[2.2.1]hept-2-yl), 8-azabicyclo[3.2.1]oct-8-yl, octahydro-2,5-epoxypentalene, hexahydro-1H-1,4-methanocyclopenta[c]furan, aza-admantane (1-azatricyclo[3.3.1.1³′⁷]decane), and oxa-adamantane (2-oxatricyclo[3.3.1.1³′⁷]decane).

The term “4-7 membered monocyclic heterocycle” as used herein, means a four-, five-, six-, or seven-membered monocyclic heterocycle, as defined herein above.

The phenyl, the aryls, the cycloalkyls, the cycloalkenyls, the heteroaryls, and the heterocycles, including the exemplary rings, are optionally substituted unless otherwise indicated; and are attached to the parent molecular moiety through any substitutable atom contained within the ring system.

The term “heteroatom” as used herein, means a nitrogen, oxygen, and sulfur.

The term “oxo” as used herein, means a ═O group.

The term “radiolabel” means a compound of the present disclosure in which at least one of the atoms is a radioactive atom or a radioactive isotope, wherein the radioactive atom or isotope spontaneously emits gamma rays or energetic particles, for example alpha particles or beta particles, or positrons. Examples of such radioactive atoms include, but are not limited to, ³H (tritium), ¹⁴C, ¹¹C, ¹⁵O, ¹⁸F, ³⁵S, ¹²³I, and ¹²⁵I.

A moiety is described as “substituted” when a non-hydrogen radical is in the place of hydrogen radical of any substitutable atom of the moiety. Thus, for example, a substituted heterocycle moiety is a heterocycle moiety in which at least one non-hydrogen radical is in the place of a hydrogen radical on the heterocycle. It should be recognized that if there are more than one substitution on a moiety, each non-hydrogen radical may be identical or different (unless otherwise stated).

If a moiety is described as being “optionally substituted,” the moiety may be either (1) not substituted or (2) substituted. If a moiety is described as being optionally substituted with up to a particular number of non-hydrogen radicals, that moiety may be either (1) not substituted; or (2) substituted by up to that particular number of non-hydrogen radicals or by up to the maximum number of substitutable positions on the moiety, whichever is less. Thus, for example, if a moiety is described as a heteroaryl optionally substituted with up to 3 non-hydrogen radicals, then any heteroaryl with less than 3 substitutable positions would be optionally substituted by up to only as many non-hydrogen radicals as the heteroaryl has substitutable positions. To illustrate, tetrazolyl (which has only one substitutable position) would be optionally substituted with up to one non-hydrogen radical. To illustrate further, if an amino nitrogen is described as being optionally substituted with up to 2 non-hydrogen radicals, then a primary amino nitrogen will be optionally substituted with up to 2 non-hydrogen radicals, whereas a secondary amino nitrogen will be optionally substituted with up to only 1 non-hydrogen radical.

The terms “treat”, “treating”, and “treatment” refer to a method of alleviating or abrogating a disease and/or its attendant symptoms. In certain embodiments, “treat,” “treating,” and “treatment” refer to ameliorating at least one physical parameter, which may not be discernible by the subject. In yet another embodiment, “treat”, “treating”, and “treatment” refer to modulating the disease or disorder, either physically (for example, stabilization of a discernible symptom), physiologically (for example, stabilization of a physical parameter), or both. In a further embodiment, “treat”, “treating”, and “treatment” refer to slowing the progression of the disease or disorder.

The terms “prevent”, “preventing”, and “prevention” refer to a method of preventing the onset of a disease and/or its attendant symptoms or barring a subject from acquiring a disease. As used herein, “prevent”, “preventing” and “prevention” also include delaying the onset of a disease and/or its attendant symptoms and reducing a subject's risk of acquiring or developing a disease or disorder.

The phrase “therapeutically effective amount” means an amount of a compound, or a pharmaceutically acceptable salt thereof, sufficient to prevent the development of or to alleviate to some extent one or more of the symptoms of the condition or disorder being treated when administered alone or in conjunction with another therapeutic agent for treatment in a particular subject or subject population. The “therapeutically effective amount” may vary depending on the compound, the disease and its severity, and the age, weight, health, etc., of the subject to be treated. For example in a human or other mammal, a therapeutically effective amount may be determined experimentally in a laboratory or clinical setting, or may be the amount required by the guidelines of the United States Food and Drug Administration, or equivalent foreign agency, for the particular disease and subject being treated.

The term “subject” is defined herein to refer to animals such as mammals, including, but not limited to, primates (e.g., humans), cows, sheep, goats, pigs, horses, dogs, cats, rabbits, rats, mice and the like. In one embodiment, the subject is a human. The terms “human,” “patient,” and “subject” are used interchangeably herein.

### Compounds

Compounds of the present disclosure have the general Formula (I) as described above.

Particular values of variable groups are as follows. Such values may be used where appropriate with any of the other values, definitions, claims or embodiments defined hereinbefore or hereinafter.

### Formula (I)

One embodiment pertains to compounds of Formula (I), or pharmaceutically acceptable salts thereof,

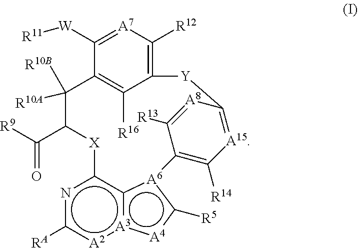

wherein
A² is CR², A³ is N, A⁴ is CR^{4a}, and A⁶ is C; or
A² is CR², A³ is N, A⁴ is O or S, and A⁶ is C; or
A² is N, A³ is C, A⁴ is O or S and A⁶ is C; or
A² is N, A³ is C, A⁴ is CR^{4a}, and A⁶ is N;
R^{A} is hydrogen, CH₃, halogen, CN, CH₂F, CHF₂, or CF₃;
X is O, or N(R^{x2}); wherein R^{x2} is hydrogen, C₁-C₃ alkyl, or unsubstituted cyclopropyl;
Y is (CH₂)ₘ, —CH═CH—(CH₂)ₙ—, —(CH₂)ₚ—CH═CH—, or —(CH₂)_{q}—CH═CH—(CH₂)ᵣ—; wherein 0, 1, 2, or 3 CH₂ groups are each independently replaced by O, N(R^{ya}), C(R^{ya})(R^{yb}), C(O), NC(O)R^{ya}, or S(O)₂;
m is 2, 3, 4, or 5;
n is 1, 2, or 3;
p is 1, 2, or 3;
q is 1 or 2; and
r is 1 or 2; wherein the sum of q and r is 2 or 3;
R^{ya}, at each occurrence, is independently hydrogen, C₂-C₆ alkenyl, C₂-C₆ alkynyl, G¹, C₁-C₆ alkyl, or C₁-C₆ haloalkyl; wherein the C₂-C₆ alkenyl, C₂-C₆ alkynyl, C₁-C₆ alkyl, and C₁-C₆ haloalkyl are optionally substituted with 1 or 2 substituents independently selected from the group consisting of oxo, —N(R^{yd})(R^{ye}), G¹, —OR^{yf}, —SR^{yg}, —S(O)₂N(R^{yd})(R^{ye}), and —S(O)₂-G¹; and
R^{yb} is C₂-C₆ alkenyl, C₂-C₆ alkynyl, G¹, C₁-C₆ alkyl, or C₁-C₆ haloalkyl; wherein the C₂-C₆ alkenyl, C₂-C₆ alkynyl, C₁-C₆ alkyl, and C₁-C₆ haloalkyl are optionally substituted with 1 or 2 substituents independently selected from the group consisting of oxo, —N(R^{yd})(R^{ye}), G¹, —OR^{yf}, —SR^{yg}, —S(O)₂N(R^{yd})(R^{ye}), and —S(O)₂-G¹; or
R^{ya} and R^{yb}, together with the carbon atom to which they are attached, form a C₃-C₇ monocyclic cycloalkyl, C₄-C₇ monocyclic cycloalkenyl, or a 4-7 membered monocyclic heterocycle; wherein the C₃-C₇ monocyclic cycloalkyl, C₄-C₇ monocyclic cycloalkenyl, and the 4-7 membered monocyclic heterocycle are each optionally substituted with 1 —OR^{m} and 0, 1, 2, or 3 independently selected R^{s} groups;
R^{yd}, R^{ye}, R^{yf}, and R^{yg}, at each occurrence, are each independently hydrogen, G¹, C₁-C₆ alkyl, or C₁-C₆ haloalkyl; wherein the C₁-C₆ alkyl and the C₁-C₆ haloalkyl are optionally substituted with one substituent selected from the group consisting of G¹, —OR^{yh}, —SR^{yh}, —SO₂R^{yh}, and —N(R^{yi})(R^{yk});
G¹, at each occurrence, is piperazinyl, piperidinyl, pyrrolidinyl, thiomorpholinyl, tetrahydropyranyl, morpholinyl, or oxetanyl; wherein each G¹ is optionally substituted with 1 —OR^{m} and 0, 1, 2, or 3 substituents independently selected from the group consisting of G², —(C₁-C₆ alkylenyl)-G², and R^{s};
G², at each occurrence, is a C₃-C₇ monocyclic cycloalkyl, C₄-C₇ monocyclic cycloalkenyl, oxetanyl, or morpholinyl; wherein each G² is optionally substituted with 1 independently selected R^{t} groups;
R² is independently hydrogen, halogen, CH₃, or CN;
R^{4a}, at each occurrence, is independently hydrogen, halogen, CN, C₂-C₄ alkenyl, C₂-C₄ alkynyl, C₁-C₄ alkyl, C₁-C₄ haloalkyl, G^{A}, C₁-C₄ alkyl-G^{A}, or C₁-C₄ alkyl-O-G^{A}; wherein each G^{A} is independently C₆-C₁₀ aryl, C₃-C₇ monocyclic cycloalkyl, C₄-C₇ monocyclic cycloalkenyl, or 4-7 membered heterocycle; wherein each G^{A} is optionally substituted with 1, 2, or 3 R^{u} groups;
R⁵ is independently hydrogen, halogen, G³, C₁-C₆ alkyl, C₂-C₆ alkenyl, or C₂-C₆ alkynyl; wherein the C₁-C₆ alkyl, C₂-C₆ alkenyl, and C₂-C₆ alkynyl are each optionally substituted with one G³;
G³, at each occurrence, is independently C₆-C₁ aryl, 5-11 membered heteroaryl, C₃-C₁₁ cycloalkyl, cycloalkenyl, oxetanyl, or 2-oxaspiro[3.3]heptanyl; wherein each G³ is optionally substituted with 1, 2, or 3 R^{v} groups;
A⁷ is N or CR⁷;
A⁸ is N or CR⁸;
A¹⁵ is N or CR¹⁵;
R⁷, R¹² and R¹⁶ are each independently hydrogen, halogen, C₁-C₄ alkyl, C₁-C₄ haloalkyl, —CN, —OR^{7a}, —SR^{7a}, or —N(R^{7b})(R^{7c});
R⁸, R¹³, R¹⁴, and R¹⁵, are each independently hydrogen, halogen, C₁-C₄ alkyl, C₁-C₄ haloalkyl, —CN, —OR^{8a}, —N(R^{8b})(R^{8c}), or C₃-C₄ monocyclic cycloalkyl; wherein the C₃-C₄ monocyclic cycloalkyl is optionally substituted with one or two substituents independently selected from the group consisting of halogen, C₁-C₃ alkyl, and C₁-C₃ haloalkyl; or
R⁸ and R¹³ are each independently hydrogen, halogen, C₁-C₄ alkyl, C₁-C₄ haloalkyl, —CN, —OR^{8a}, —SR^{8a}, —N(R^{8b})(R^{8c}), or C₃-C₄ monocyclic cycloalkyl; wherein the C₃-C₄ monocyclic cycloalkyl is optionally substituted with one or two substituents independently selected from the group consisting of halogen, C₁-C₃ alkyl, and C₁-C₃ haloalkyl; and
R¹⁴ and R¹⁵, together with the carbon atoms to which they are attached, form a monocyclic ring selected from the group consisting of benzene, cyclobutane, cyclopentane, and pyridine; wherein the monocyclic ring is optionally substituted with 1, 2, or 3 substituents independently selected from the group consisting of halogen, C₁-C₄ alkyl, C₁-C₄ haloalkyl, —CN, —OR^{8a}, —SR^{8a}, and —N(R^{8b})(R^{8c});
R⁹ is —OH, —O—C₁-C₄ alkyl, —O—CH₂—OC(O)(C₁-C₆ alkyl), —NHOH,

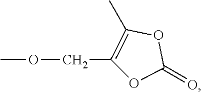

or —N(H)S(O)₂—(C₁-C₆ alkyl);
R^{10A} and R^{10B}, are each independently hydrogen, C₁-C₃ alkyl, or C₁-C₃ haloalkyl; or R^{10A} and R^{10B}, together with the carbon atom to which they are attached, form a cyclopropyl; wherein the cyclopropyl is optionally substituted with one or two substituents independently selected from the group consisting of halogen and CH₃;
W is —CH═CH—, C₁-C₄ alkyl, —O—CHF—, -L¹-CH₂—, or —CH₂-L¹-; wherein L¹ at each occurrence, is independently O, S, S(O), S(O)₂, S(O)₂N(H), N(H), or N(C₁-C₃ alkyl);
R¹¹ is a C₆-C₁₀ aryl or a 5-11 membered heteroaryl; wherein each R¹¹ is optionally substituted with 1, 2, or 3 independently selected R^{w} groups;
R^{w}, at each occurrence, is independently C₁-C₆ alkyl, C₂-C₆ alkenyl, C₂-C₆ alkynyl, halogen, C₁-C₆ haloalkyl, —CN, NO₂, —OR^{11a}, —SR^{11b}, —S(O)₂R^{11b}, —S(O)₂N(R^{11c})₂, —C(O)R^{11a}, —C(O)N(R^{11c})₂, —N(R^{11c})₂, —N(R^{11c})C(O)R^{11b}, —N(R^{11c})S(O)₂R^{11b}, —N(R^{11c})C(O)O(R^{11b}), —N(R^{11c})C(O)N(R^{11c})₂, G⁴, —(C₁-C₆ alkylenyl)-OR^{11a}, —(C₁-C₆alkylenyl)-OC(O)N(R^{11c})₂, —(C₁-C₆ alkylenyl)-SR^{11a}, —(C₁-C₆ alkylenyl)-S(O)₂R^{11b}, —(C₁-C₆ alkylenyl)-S(O)₂N(R^{11c})₂, —(C₁-C₆ alkylenyl)-C(O)R^{11a}, —(C₁-C₆ alkylenyl)-C(O)N(R^{11c})₂, —(C₁-C₆ alkylenyl)-N(R^{11c})₂, —(C₁-C₆ alkylenyl)-N(R^{11c})C(O)R^{11b}, —(C₁-C₆alkylenyl)-N(R^{11c})S(O)₂R^{11b}, —(C₁-C₆ alkylenyl)-N(R^{11c})C(O)O(R^{11b}), —(C₁-C₆alkylenyl)-N(R^{11c})C(O)N(R^{11c})₂, —(C₁-C₆ alkylenyl)-CN, or —(C₁-C₆ alkylenyl)-G⁴;
R^{11a} and R^{11c}, at each occurrence, are each independently hydrogen, C₁-C₆ alkyl, C₂-C₆ alkenyl, C₁-C₆ haloalkyl, G⁴, —(C₂-C₆alkylenyl)-OR^{11d}, —(C₂-C₆ alkylenyl)-N(R^{11e})₂, or —(C₂-C₆ alkylenyl)-G⁴;
R^{11b}, at each occurrence, is independently C₁-C₆ alkyl, C₂-C₆ alkenyl, C₁-C₆ haloalkyl, G⁴, —(C₂-C₆ alkylenyl)-OR^{11d}, —(C₂-C₆ alkylenyl)-N(R^{11e})₂, or —(C₂-C₆ alkylenyl)-G⁴;
G⁴, at each occurrence, is independently phenyl, monocyclic heteroaryl, C₃-C₁₁ cycloalkyl, C₄-C₁₁ cycloalkenyl, oxetanyl, tetrahydrofuranyl, tetrahydropyranyl, morpholinyl, 2,6-dioxa-9-azaspiro[4.5]decanyl, 2-oxa-5-azabicyclo[2.2.1]heptanyl, 3-oxa-8-azabicyclo[3.2.1]octanyl, piperidinyl, azetidinyl, dihydropyranyl, tetrahydropyridinyl, dihydropyrrolyl, or pyrrolidinyl; wherein each G⁴ is optionally substituted with 1 —OR^{m} and 0, 1, 2, 3, or 4 substituents independently selected from the group consisting of G⁵, R^{y}, —(C₁-C₆ alkylenyl)-G⁵, and -L²-(C₁-C₆ alkylenyl), -G⁵;
L² is O, C(O), N(H), N(C₁-C₆ alkyl), NHC(O), C(O)O, S, S(O), or S(O)₂;
s is 0 or 1;
G⁵, at each occurrence, is independently phenyl, monocyclic heteroaryl, C₃-C₇ monocyclic cycloalkyl, C₄-C₇ monocyclic cycloalkenyl, or piperazine; wherein each G⁵ is optionally substituted with 1 independently selected —OR^{m} or R^{z} group;
R^{s}, R^{t}, R^{u}, R^{v}, R^{y}, and R^{z}, at each occurrence, are each independently C₁-C₆ alkyl, C₂-C₆ alkenyl, C₂-C₆ alkynyl, halogen, C₁-C₆ haloalkyl, —CN, oxo, NO₂, P(O)(R^{k})₂, —OC(O)R^{k}, —OC(O)N(R^{j})₂, —SR^{j}, —S(O)₂R^{k}, —S(O)₂N(R^{j})₂, —C(O)R^{j}, —C(O)N(R^{j})₂, —N(R^{j})₂, —N(R^{j})C(O)R^{k}, —N(R^{j})S(O)₂R^{k}, —N(R^{j})C(O)O(R^{k}), —N(R^{j})C(O)N(R^{j})₂, —(C₁-C₆ alkylenyl)-OR^{j}, —(C₁-C₆ alkylenyl)-OC(O)N(R^{j})₂, —(C₁-C₆alkylenyl)-SR^{j}, —(C₁-C₆alkylenyl)-S(O)₂R^{k}, —(C₁-C₆ alkylenyl)-S(O)₂N(R^{j})₂, —(C₁-C₆ alkylenyl)-C(O)R^{j}, —(C₁-C₆ alkylenyl)-C(O)N(R^{j})₂, —(C₁-C₆ alkylenyl)-N(R^{j})₂, —(C₁-C₆alkylenyl)-N(R^{j})C(O)R^{k}, —(C₁-C₆ alkylenyl)-N(R^{j})S(O)₂R^{k}, —(C₁-C₆ alkylenyl)-N(R^{j})C(O)O(R^{k}), —(C₁-C₆ alkylenyl)-N(R^{j})C(O)N(R^{j})₂, or —(C₁-C₆ alkylenyl)-CN;
R^{m} is hydrogen, C₁-C₆ alkyl, C₁-C₆ haloalkyl, —(C₂-C₆alkylenyl)-OR^{j}, or —(C₂-C₆ alkylenyl)-N(R^{j})₂;
R^{yh}, R^{yi}, R^{yk}, R^{7a}, R^{7b}, R^{7c}, R^{8a}, R^{8b}, R^{8c}, R^{11d}, R^{11e}, and R^{j}, at each occurrence, are each independently hydrogen, C₁-C₆ alkyl, or C₁-C₆ haloalkyl; and
R^{k}, at each occurrence, is independently C₁-C₆ alkyl or C₁-C₆ haloalkyl.

In one embodiment of Formula (I), A² is CR², A³ is N, A⁴ is CR^{4a}, and A⁶ is C; or A² is CR², A³ is N, A⁴ is O or S, and A⁶ is C; or A² is N, A³ is C, A⁴ is O or S and A⁶ is C; or A² is N, A³ is C, A⁴ is CR^{4a}, and A⁶ is N. In another embodiment of Formula (I), A² is CR², A³ is N, A⁴ is CR^{4a}, and A⁶ is C. In another embodiment of Formula (I), A² is CH, A³ is N, A⁴ is CH, and A⁶ is C. In another embodiment of Formula (I), A² is CR², A³ is N, A⁴ is CR^{4a}, A⁶ is C, R² is H, and R^{4a} is halogen. In another embodiment of Formula (I), A² is CR², A³ is N, A⁴ is CR^{4a}, A⁶ is C, R² is H, and R^{4a} is Cl. In another embodiment of Formula (I), A² is CR², A³ is N, A⁴ is O or S, and A⁶ is C. In another embodiment of Formula (I), A² is N, A³ is C, A⁴ is O, and A⁶ is C. In another embodiment of Formula (I), A² is N, A³ is C, A⁴ is S, and A⁶ is C. In another embodiment of Formula (I), A² is N, A³ is C, A⁴ is CR^{4a}, and A⁶ is N.

In one embodiment of Formula (I), R^{A} is hydrogen, CH₃, halogen, CN, CH₂F, CHF₂, or CF₃. In another embodiment of Formula (I), R^{A} is hydrogen.

In one embodiment of Formula (I), X is O, or N(R^{x2}); wherein R^{x2} is hydrogen, C₁-C₃ alkyl, or unsubstituted cyclopropyl. In another embodiment of Formula (I), X is O.

In one embodiment of Formula (I), Y is (CH₂)ₘ, —CH═CH—(CH₂)ₙ—, —(CH₂)ₚ—CH═CH—, or —(CH₂)_{q}—CH═CH—(CH₂)ᵣ—; wherein 0, 1, 2, or 3 CH₂ groups are each independently replaced by O, N(R^{ya}), C(R^{ya})(R^{yb}), C(O), NC(O)R^{ya}, or S(O)₂; and m is 2, 3, 4, or 5. In another embodiment of Formula (I), Y is (CH₂)ₘ; wherein 1, 2, or 3 CH₂ groups are each independently replaced by O, N(R^{ya}), C(R^{ya})(R^{yb}), C(O), or NC(O)R^{ya}; and m is 3 or 4. In another embodiment of Formula (I), Y is (CH₂)ₘ; wherein 1 CH₂ group is independently replaced by N(R^{ya}); and m is 3. In another embodiment of Formula (I), Y is (CH₂)ₘ; wherein 2 CH₂ groups are each independently replaced by O and 1 CH₂ group is replaced by C(R^{ya})(R^{yb}); and m is 4. In another embodiment of Formula (I), Y is

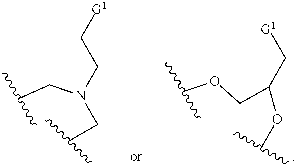

In another embodiment of Formula (I), Y is

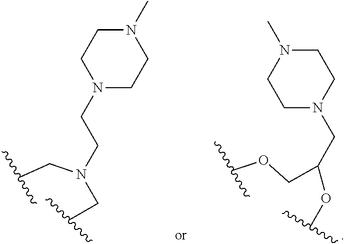

In one embodiment of Formula (I), R^{ya}, at each occurrence, is independently hydrogen, C₂-C₆ alkenyl, C₂-C₆ alkynyl, G¹, C₁-C₆ alkyl, or C₁-C₆ haloalkyl; wherein the C₂-C₆ alkenyl, C₂-C₆ alkynyl, C₁-C₆ alkyl, and C₁-C₆ haloalkyl are optionally substituted with 1 or 2 substituents independently selected from the group consisting of oxo, —N(R^{yd})(R^{ye}), G¹, —OR^{yf}, —SR^{yg}, —S(O)₂N(R^{yd})(R^{ye}), and —S(O)₂-G¹; and R^{yb} is C₂-C₆ alkenyl, C₂-C₆ alkynyl, G¹, C₁-C₆ alkyl, or C₁-C₆ haloalkyl; wherein the C₂-C₆ alkenyl, C₂-C₆ alkynyl, C₁-C₆ alkyl, and C₁-C₆ haloalkyl are optionally substituted with 1 or 2 substituents independently selected from the group consisting of oxo, —N(R^{yd})(R^{ye}), G¹, —OR^{yf}, —SR^{yg}, —S(O)₂N(R^{yd})(R^{ye}), and —S(O)₂-G¹; or R^{ya} and R^{yb}, together with the carbon atom to which they are attached, form a C₃-C₇ monocyclic cycloalkyl, C₄-C₇ monocyclic cycloalkenyl, or a 4-7 membered monocyclic heterocycle; wherein the C₃-C₇ monocyclic cycloalkyl, C₄-C₇ monocyclic cycloalkenyl, and the 4-7 membered monocyclic heterocycle are each optionally substituted with 1 —OR^{m} and 0, 1, 2, or 3 independently selected R^{s} groups; and R^{yd}, R^{ye}, R^{yf}, and R^{yg}, at each occurrence, are each independently hydrogen, G¹, C₁-C₆ alkyl, or C₁-C₆ haloalkyl; wherein the C₁-C₆ alkyl and the C₁-C₆ haloalkyl are optionally substituted with one substituent selected from the group consisting of G¹, —OR^{yh}, —SR^{yh}, —SO₂R^{yh}, and —N(R^{yi})(R^{yk}). In another embodiment of Formula (I), R^{ya}, at each occurrence, is independently hydrogen, or C₁-C₆ alkyl; wherein the C₁-C₆ alkyl is optionally substituted with 1 or 2 substituents independently selected from the group consisting of —N(R^{yd})(R^{ye}), —OR^{yf}, or C₁-C₆ alkyl; and R^{yb} is C₁-C₆ alkyl; wherein the C₁-C₆ alkyl is optionally substituted with 1 or 2 substituents independently selected from the group consisting of —N(R^{yd})(R^{ye}), G¹, and —OR^{yf}; and R^{yd}, R^{ye}, and R^{yf}, at each occurrence, are each independently hydrogen, or C₁-C₆ alkyl; wherein the C₁-C₆ alkyl is optionally substituted with one substituent selected from the group consisting of G¹, —OR^{yh}, and SO₂R^{yh}. In another embodiment of Formula (I), R^{ya}, at each occurrence, is independently hydrogen; and R^{yb} is C₁-C₆ alkyl; wherein the C₁-C₆ alkyl is substituted with 1 G¹.

In one embodiment of Formula (I), G¹, at each occurrence, is piperazinyl, piperidinyl, pyrrolidinyl, thiomorpholinyl, tetrahydropyranyl, morpholinyl, or oxetanyl; wherein each G¹ is optionally substituted with 1 —OR^{m} and 0, 1, 2, or 3 substituents independently selected from the group consisting of G², —(C₁-C₆ alkylenyl)-G², and R^{s}. In another embodiment of Formula (I), G¹ is piperazinyl optionally substituted with 1 —OR^{m} and 0, 1, 2, or 3 substituents independently selected from the group consisting of G², —(C₁-C₆ alkylenyl)-G², and R^{s}. In another embodiment of Formula (I), G¹ is piperazinyl substituted with 1 R^{s}. In another embodiment of Formula (I), G¹ is piperazinyl substituted with 1 R^{s}; and R^{s} is C₁-C₆ alkyl. In another embodiment of Formula (I), G¹ is piperazinyl substituted with 1 R^{s}; and R^{s} is CH₃.

In one embodiment of Formula (I), G², at each occurrence, is a C₃-C₇ monocyclic cycloalkyl, C₄-C₇ monocyclic cycloalkenyl, oxetanyl, or morpholinyl; wherein each G² is optionally substituted with 1 independently selected R^{t} groups. In another embodiment of Formula (I), G², at each occurrence, is a C₃-C₇ monocyclic cycloalkyl. In another embodiment of Formula (I), G², at each occurrence, is a morpholinyl.

In one embodiment of Formula (I), R² is independently hydrogen, halogen, CH₃, or CN. In another embodiment of Formula (I), R² is independently hydrogen.

In one embodiment of Formula (I), R^{4a}, at each occurrence, is independently hydrogen, halogen, CN, C₂-C₄ alkenyl, C₂-C₄ alkynyl, C₁-C₄ alkyl, C₁-C₄ haloalkyl, G^{A}, C₁-C₄ alkyl-G^{A}, or C₁-C₄ alkyl-O-G^{A}; wherein each G^{A} is independently C₆-C₁₀ aryl, C₃-C₇ monocyclic cycloalkyl, C₄-C₇ monocyclic cycloalkenyl, or 4-7 membered heterocycle; wherein each G^{A} is optionally substituted with 1, 2, or 3 R^{u} groups. In another embodiment of Formula (I), R^{4a}, at each occurrence, is independently halogen.

In one embodiment of Formula (I), R⁵ is independently hydrogen, halogen, G³, C₁-C₆ alkyl, C₂-C₆ alkenyl, or C₂-C₆ alkynyl; wherein the C₁-C₆ alkyl, C₂-C₆ alkenyl, and C₂-C₆ alkynyl are each optionally substituted with one G³; and G³, at each occurrence, is independently C₆-C₁₀ aryl, 5-11 membered heteroaryl, C₃-C₁₁cycloalkyl, cycloalkenyl, oxetanyl, or 2-oxaspiro[3.3]heptanyl; wherein each G³ is optionally substituted with 1, 2, or 3 R^{v} groups. In another embodiment of Formula (I), R⁵ is independently hydrogen, G³, or C₂-C₆ alkynyl; and G³, at each occurrence, is independently C₆-C₁₀ aryl, or C₃-C₁₁ cycloalkyl; wherein each G³ is optionally substituted with 1, 2, or 3 R^{v} groups. In another embodiment of Formula (I), R⁵ is independently G³; and G³, at each occurrence, is independently C₆-C₁₀ aryl; wherein each G³ is optionally substituted with 1 R^{v} group. In another embodiment of Formula (I), R⁵ is independently G³; and G³, at each occurrence, is independently phenyl; wherein each G³ is optionally substituted with 1 R^{v} group; and R^{v} is halogen. In another embodiment of Formula (I), R⁵ is independently G³; and G³, at each occurrence, is independently phenyl; wherein G³ is optionally substituted with 1 R^{v} group; and R^{v} is C₁.

In one embodiment of Formula (I), A⁷ is N or CR⁷; A⁸ is N or CR⁸; and A¹⁵ is N or CR¹⁵. In another embodiment of Formula (I), R⁷, R¹² and R¹⁶ are each independently hydrogen, halogen, C₁-C₄ alkyl, C₁-C₄ haloalkyl, —CN, —OR^{7a}, —SR^{7a}, or —N(R^{7b})(R^{7c}); and R⁸, R¹³, R¹⁴, and R¹⁵, are each independently hydrogen, halogen, C₁-C₄ alkyl, C₁-C₄ haloalkyl, —CN, —OR^{8a}, —SR^{8a}, —N(R^{8b})(R^{8c}), or C₃-C₄ monocyclic cycloalkyl; wherein the C₃-C₄ monocyclic cycloalkyl is optionally substituted with one or two substituents independently selected from the group consisting of halogen, C₁-C₃ alkyl, and C₁-C₃ haloalkyl. In another embodiment of Formula (I), R⁷, R¹² and R¹⁶ are each independently hydrogen. In another embodiment of Formula (I), A⁷ is CH; A⁸ is CR⁸; and A¹⁵ is CR¹⁵; and R⁸, and R¹⁵ are each independently hydrogen, halogen, C₁-C₄ alkyl, or —OR^{8a}.

In one embodiment of Formula (I), R⁸ and R¹³ are each independently hydrogen, halogen, C₁-C₄ alkyl, C₁-C₄ haloalkyl, —CN, —OR^{8a}, —SR^{8a}, —N(R^{8b})(R^{8c}), or C₃-C₄ monocyclic cycloalkyl; wherein the C₃-C₄ monocyclic cycloalkyl is optionally substituted with one or two substituents independently selected from the group consisting of halogen, C₁-C₃ alkyl, and C₁-C₃ haloalkyl; and R¹⁴ and R¹⁵, together with the carbon atoms to which they are attached, form a monocyclic ring selected from the group consisting of benzene, cyclobutane, cyclopentane, and pyridine; wherein the monocyclic ring is optionally substituted with 1, 2, or 3 substituents independently selected from the group consisting of halogen, C₁-C₄ alkyl, C₁-C₄ haloalkyl, —CN, —OR^{8a}, —SR^{8a}, and —N(R^{8b})(R^{8c}). In another embodiment of Formula (I), R⁸ and R¹³ are each independently hydrogen, and R¹⁴ and R¹⁵, together with the carbon atoms to which they are attached form benzene.

In one embodiment of Formula (I), R⁹ is —OH, —O—C₁-C₄ alkyl, —O—CH₂—OC(O)(C₁-C₆ alkyl), —NHOH,

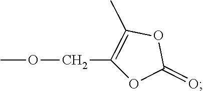

or —N(H)S(O)₂—(C₁-C₆ alkyl). In another embodiment of Formula (I), R⁹ is —OH.

In one embodiment of Formula (I), R^{10A} and R^{10B}, are each independently hydrogen, C₁-C₃ alkyl, or C₁-C₃ haloalkyl; or R^{10A} and R^{10B}, together with the carbon atom to which they are attached, form a cyclopropyl; wherein the cyclopropyl is optionally substituted with one or two substituents independently selected from the group consisting of halogen and CH₃. In another embodiment of Formula (I), R^{10A} and R^{10B} are each independently hydrogen.

In one embodiment of Formula (I),
R^{A} is hydrogen;
R⁹ is —OH;
R^{10A} and R^{10B}, are each independently hydrogen; and
R⁷, R¹² and R¹⁶ are each independently hydrogen.

In one embodiment of Formula (I), W is —CH═CH—, C₁-C₄ alkyl, —O—CHF—, -L¹-CH₂—, or —CH₂-L¹-; wherein L¹ at each occurrence, is independently O, S, S(O), S(O)₂, S(O)₂N(H), N(H), or N(C₁-C₃ alkyl). In another embodiment of Formula (I), W is —O—CHF—, or -L¹-CH₂—; wherein L¹ at each occurrence, is independently O. In another embodiment of Formula (I), W is -L¹-CH₂—; wherein at each occurrence, is independently O.

In one embodiment of Formula (I), R¹¹ is a C₆-C₁₀ aryl or a 5-11 membered heteroaryl; wherein each R¹¹ is optionally substituted with 1, 2, or 3 independently selected R^{w} groups. In another embodiment of Formula (I), is a C₆-C₁₀ aryl or a 5-11 membered heteroaryl; wherein each R¹¹ is optionally substituted with 1 independently selected R^{w} groups. In another embodiment of Formula (I), W is —O—CH₂—, and R¹¹ is pyrimidinyl, optionally substituted with 1, 2, or 3 independently selected R^{w} groups. In another embodiment of Formula (I), W is —O—CH₂—; and R¹¹ is pyrimidinyl, optionally substituted with 1, 2, or 3 independently selected R^{w} groups; and R^{w}, at each occurrence, is independently C₁-C₆ alkyl, —OR^{11a}, or G⁴.

In one embodiment of Formula (I), R^{11a} and R^{11c}, at each occurrence, are each independently hydrogen, C₁-C₆ alkyl, C₂-C₆ alkenyl, C₁-C₆ haloalkyl, G⁴, —(C₂-C₆alkylenyl)-OR^{11d}, —(C₂-C₆ alkylenyl)-N(R^{11c})₂, or —(C₂-C₆ alkylenyl)-G⁴; and R^{11b}, at each occurrence, is independently C₁-C₆ alkyl, C₂-C₆ alkenyl, C₁-C₆ haloalkyl, G⁴, —(C₂-C₆alkylenyl)-OR^{11d}, —(C₂-C₆alkylenyl)-N(R^{11e})₂, or —(C₂-C₆ alkylenyl)-G⁴. In another embodiment of Formula (I), R^{11a} is C₁-C₆ alkyl or C₁-C₆ haloalkyl.

In one embodiment of Formula (I), G⁴, at each occurrence, is independently phenyl, monocyclic heteroaryl, C₃-C₁₁ cycloalkyl, C₄-C₁₁ cycloalkenyl, oxetanyl, tetrahydrofuranyl, tetrahydropyranyl, morpholinyl, 2,6-dioxa-9-azaspiro[4.5]decanyl, 2-oxa-5-azabicyclo[2.2.1]heptanyl, 3-oxa-8-azabicyclo[3.2.1]octanyl, piperidinyl, azetidinyl, dihydropyranyl, tetrahydropyridinyl, dihydropyrrolyl, or pyrrolidinyl; wherein each G⁴ is optionally substituted with 1 —OR^{m} and 0, 1, 2, 3, or 4 substituents independently selected from the group consisting of G⁵, R^{y}, —(C₁-C₆ alkylenyl)-G⁵, and -L²-(C₁-C₆alkylenyl)ₛ-G⁵; and L² is O, C(O), N(H), N(C₁-C₆ alkyl), NHC(O), C(O)O, S, S(O), or S(O)₂; and s is 0 or 1. In another embodiment of Formula (I), G⁴, at each occurrence, is independently phenyl, monocyclic heteroaryl, C₃-C₁₁ cycloalkyl, oxetanyl, tetrahydrofuranyl, tetrahydropyranyl, morpholinyl, 2,6-dioxa-9-azaspiro[4.5]decanyl, 2-oxa-5-azabicyclo[2.2.1]heptanyl, 3-oxa-8-azabicyclo[3.2.1]octanyl, or pyrrolidinyl; wherein each G⁴ is optionally substituted with 1 —OR^{m} and 0, 1, 2, 3, or 4 substituents independently selected from the group consisting of R^{y}, and -L²-(C₁-C₆alkylenyl)ₛ-G⁵; L² is O or C(O)O; and s is 0 or 1. In another embodiment of Formula (I), G⁴, at each occurrence, is independently phenyl optionally substituted with 1 —OR^{m} and 0, 1, 2, 3, or 4 substituents independently selected from the group consisting of R^{y}, and -L²-(C₁-C₆alkylenyl)ₛ-G⁵; L² is O or C(O)O; and s is 0 or 1. In another embodiment of Formula (I), G⁴, at each occurrence, is independently tetrahydrofuranyl optionally substituted with 1 —OR^{m} and 0, 1, 2, 3, or 4 substituents independently selected from the group consisting of R^{y}, and -L²-(C₁-C₆alkylenyl)ₛ-G⁵; L² is O or C(O)O; and s is 0 or 1. In another embodiment of Formula (I), G⁴, at each occurrence, is independently tetrahydropyranyl optionally substituted with 1 —OR^{m} and 0, 1, 2, 3, or 4 substituents independently selected from the group consisting of R^{y}, and -L²-(C₁-C₆alkylenyl)ₛ-G⁵; L² is O or C(O)O; and s is 0 or 1. In another embodiment of Formula (I), G⁴, at each occurrence, is independently phenyl optionally substituted with 1 —OCH₃.

In one embodiment of Formula (I), G⁵, at each occurrence, is independently phenyl, monocyclic heteroaryl, C₃-C₇ monocyclic cycloalkyl, C₄-C₇ monocyclic cycloalkenyl, or piperazine; wherein each G⁵ is optionally substituted with 1 independently selected —OR^{m} or R^{z} group. In another embodiment of Formula (I), G⁵, at each occurrence, is independently phenyl optionally substituted with 1 independently selected R^{z} group.

In one embodiment of Formula (I),
A² is CH;
A³ is N;
A⁴ is CH;
A⁶ is C;
R^{A} is hydrogen;
X is O;
R⁹ is —OH;
R^{10A} and R^{10B}, are each independently hydrogen; and
R⁷, R¹² and R¹⁶ are each independently hydrogen.

In one embodiment of Formula (I),
A² is N;
A³ is C;
A⁴ is O;
A⁶ is C;
R^{A} is hydrogen;
X is O;
R⁹ is —OH;
R^{10A} and R^{10B}, are each independently hydrogen; and
R⁷, R¹² and R¹⁶ are each independently hydrogen.

In one embodiment of Formula (I),
A² is N;
A³ is C;
A⁴ is S;
A⁶ is C;
R^{A} is hydrogen;
X is O;
R⁹ is —OH;
R^{10A} and R^{10B}, are each independently hydrogen; and
R⁷, R¹² and R¹⁶ are each independently hydrogen.

In one embodiment of Formula (I),
A² is N;
A³ is C;
A⁴ is S;
A⁶ is C;
R^{A} is hydrogen;
X is O;
R⁹ is —OH;
R^{10A} and R^{10B}, are each independently hydrogen;
R⁷, R¹² and R¹⁶ are each independently hydrogen;
Y is (CH₂)ₘ; wherein 1 CH₂ group is independently replaced by N(R^{ya}); and
m is 3.

In one embodiment of Formula (I),
A² is N;
A³ is C;
A⁴ is S;
A⁶ is C;
R^{A} is hydrogen;
X is O;
R⁹ is —OH;
R^{10A} and R^{10B}, are each independently hydrogen;
R⁷, R¹² and R¹⁶ are each independently hydrogen;
Y is (CH₂)ₘ; wherein 2 CH₂ groups are each independently replaced by O and 1 CH₂ group is
replaced by C(R^{ya})(R^{yb}); and
m is 4.

In one embodiment of Formula (I),
A² is CH;
A³ is N;
A⁴ is CH;
A⁶ is C;
R^{A} is hydrogen;
X is O;
R⁹ is —OH;
R^{10A} and R^{10B}, are each independently hydrogen;
R⁷, R¹² and R¹⁶ are each independently hydrogen;
Y is (CH₂)ₘ; wherein 1 CH₂ group is independently replaced by N(R^{ya});
m is 3; and
G¹ is piperazinyl substituted with 1 R^{s}.

In one embodiment of Formula (I),
A² is CH;
A³ is N;
A⁴ is CH;
A⁶ is C;
R^{A} is hydrogen;
X is O;
R⁹ is —OH;
R^{10A} and R^{10B}, are each independently hydrogen;
R⁷, R¹² and R¹⁶ are each independently hydrogen;
Y is (CH₂)ₘ; wherein 2 CH₂ groups are each independently replaced by O and 1 CH₂ group is replaced by C(R^{ya})(R^{yb});
m is 4; and
G¹ is piperazinyl substituted with 1 R^{s}.

In one embodiment of Formula (I),
A² is CH;
A³ is N;
A⁴ is CH;
A⁶ is C;
R^{A} is hydrogen;
X is O;
R⁹ is —OH;
R^{10A} and R^{10B}, are each independently hydrogen;
R⁷, R¹² and R¹⁶ are each independently hydrogen;
Y is (CH₂)ₘ; wherein 1 CH₂ group is independently replaced by N(R^{ya});
m is 3;
G¹ is piperazinyl substituted with 1 R^{s};
W is -L¹-CH₂—; and
L¹ is independently O.

In one embodiment of Formula (I),
A² is CH;
A³ is N;
A⁴ is CH;
A⁶ is C;
R^{A} is hydrogen;
X is O;
R⁹ is —OH;
R^{10A} and R^{10B}, are each independently hydrogen;
R⁷, R¹² and R¹⁶ are each independently hydrogen;
Y is (CH₂)ₘ; wherein 2 CH₂ groups are each independently replaced by O and 1 CH₂ group is replaced by C(R^{ya})(R^{yb});
m is 4;
G¹ is piperazinyl substituted with 1 R^{s};
W is -L¹-CH₂—; and
L¹ is independently O.

In one embodiment of Formula (I),
A² is CH;
A³ is N;
A⁴ is CH;
A⁶ is C;
R^{A} is hydrogen;
X is O;
R⁹ is —OH;
R^{10A} and R^{10B}, are each independently hydrogen;
R⁷, R¹² and R¹⁶ are each independently hydrogen;
Y is (CH₂)ₘ; wherein 1 CH₂ group is independently replaced by N(R^{ya});

m is 3;

G¹ is piperazinyl substituted with 1 R^{s};
W is -L¹-CH₂—;
L¹ is independently O;
W is —O—CH₂—, and
R¹¹ is pyrimidinyl, optionally substituted with 1, 2, or 3 independently selected R^{w} groups.

One embodiment pertains to compounds of Formula (I), or pharmaceutically acceptable salts thereof,

wherein
A² is CR², A³ is N, A⁴ is CR^{4a}, and A⁶ is C; or
A² is N, A³ is C, A⁴ is O or S and A⁶ is C;
R^{A} is hydrogen;
X is O;
Y is (CH₂)ₘ, wherein 0, 1, 2, or 3 CH₂ groups are each independently replaced by O, N(R^{ya}), C(R^{ya})(R^{yb}), C(O), or NC(O)R^{ya};
m is 3, or 4;
R^{ya}, at each occurrence, is independently hydrogen, or C₁-C₆ alkyl; wherein the C₁-C₆ alkyl is optionally substituted with 1 substituent independently selected from the group consisting of —N(R^{yd})(R^{ye}), G¹, and —OR^{yf};
R^{yb} is C₁-C₆ alkyl; wherein the C₁-C₆ alkyl is optionally substituted with 1 substituent independently selected from the group consisting of —N(R^{yd})(R^{ye}), G¹, and —OR^{yf};
R^{yd}, R^{ye}, and R^{yf}, at each occurrence, are each independently hydrogen, or C₁-C₆ alkyl; wherein the C₁-C₆ alkyl is optionally substituted with one substituent selected from the group consisting of G¹, —OR^{yh}, and —SO₂R^{yh};
G¹, at each occurrence, is piperazinyl, piperidinyl, pyrrolidinyl, thiomorpholinyl, tetrahydropyranyl, morpholinyl, or oxetanyl; wherein each G¹ is optionally substituted with 1 —OR^{m} and 0, 1, 2, or 3 substituents independently selected from the group consisting of G² and R^{s};
G², at each occurrence, is a C₃-C₇ monocyclic cycloalkyl or morpholinyl; wherein each G² is optionally substituted with 1 independently selected R^{t} groups;
R² is independently hydrogen;
R^{4a}, at each occurrence, is independently halogen;
R⁵ is independently hydrogen, G³, or C₂-C₆ alkynyl;
G³, at each occurrence, is independently C₆-C₁₀ aryl, or C₃-C₁₁ cycloalkyl; wherein each G³ is optionally substituted with 1, 2, or 3 R^{v} groups;
A⁷ is CR⁷;
A⁸ is CR⁸;
A¹⁵ is CR¹⁵;
R⁷, R¹² and R¹⁶ are each independently hydrogen;
R⁸, R¹³, R¹⁴, and R¹⁵, are each independently hydrogen, halogen, C₁-C₄ alkyl, or —OR^{8a}; or
R⁸ and R¹³ are each independently hydrogen; and
R¹⁴ and R¹⁵, together with the carbon atoms to which they are attached, form benzene;
R⁹ is —OH, —O—C₁-C₄ alkyl, —O—CH₂—OC(O)(C₁-C₆ alkyl), —NHOH, or

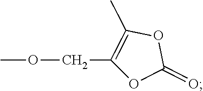

R^{10A} and R^{10B}, are each independently hydrogen;
W is —O—CHF—, -L¹-CH₂—; wherein L¹ at each occurrence, is independently O;
R¹¹ is a C₆-C¹⁰ aryl or a 5-11 membered heteroaryl; wherein each R¹¹ is optionally substituted with 1, 2, or 3 independently selected R^{w} groups;
R^{w}, at each occurrence, is independently C₁-C₆ alkyl, —OR^{11a}, or G⁴;
R^{11a} and R^{11c}, at each occurrence, are each independently hydrogen, C₁-C₆ alkyl, or C₁-C₆ haloalkyl;
G⁴, at each occurrence, is independently phenyl, monocyclic heteroaryl, C₃-C₁₁ cycloalkyl, oxetanyl, tetrahydrofuranyl, tetrahydropyranyl, morpholinyl, 2,6-dioxa-9-azaspiro[4.5]decanyl, 2-oxa-5-azabicyclo[2.2.1]heptanyl, 3-oxa-8-azabicyclo[3.2.1]octanyl, or pyrrolidinyl; wherein each G⁴ is optionally substituted with 1 —OR^{m} and 0, 1, 2, 3, or 4 substituents independently selected from the group consisting of R^{y}, and -L²-(C₁-C₆ alkylenyl)ₛ-G⁵;
L² is O, or C(O)O;
s is 0 or 1;
G⁵, at each occurrence, is independently phenyl; wherein each G⁵ is optionally substituted with 1 independently selected or R^{z} group;
R^{s}, R^{v}, R^{y}, and R^{z}, at each occurrence, are each independently C₁-C₆ alkyl, halogen, C₁-C₆ haloalkyl, —CN, oxo, P(O)(R^{k})₂, —S(O)₂R^{k}, —C(O)R^{j}, —N(R^{j})₂, —(C₁-C₆ alkylenyl)-OR^{j}, or —(C₁-C₆ alkylenyl)-S(O)₂R^{k};

R^{m} is hydrogen, C₁-C₆ alkyl, C₁-C₆ haloalkyl, or —(C₂-C₆ alkylenyl)-OR^{j};
R^{yh}, R^{8a}, and R^{j}, at each occurrence, are each independently hydrogen, C₁-C₆ alkyl, or C₁-C₆ haloalkyl; and
R^{k}, at each occurrence, is independently C₁-C₆ alkyl.

Exemplary compounds of Formula (I) include, but are not limited to:
(7R,20S)-18-chloro-1-(4-fluorophenyl)-10-{[2-(2-methoxyphenyl)pyrimidin-4-yl]methoxy}-19-methyl-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylic acid;
(5R)-21-(4-fluorophenyl)-8-{[2-(2-methoxyphenyl)pyrimidin-4-yl]methoxy}-13-[2-(4-methylpiperazin-1-yl)ethyl]-5,6,13,14-tetrahydro-12H-15,20-etheno-11,7-(metheno)-4-oxa-22-thia-1,3,13-triazabenzo[16,17]cyclooctadeca[1,2,3-cd]indene-5-carboxylic acid;
(7R,20S)-1-(4-fluorophenyl)-10-{[2-(2-methoxyphenyl)pyrimidin-4-yl]methoxy}-18,19-dimethyl-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylic acid;
(7R,20S)-1-(4-fluorophenyl)-10-{[2-(2-methoxyphenyl)pyrimidin-4-yl]methoxy}-19-methyl-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylic acid;
(7R,20S)-18,19-difluoro-1-(4-fluorophenyl)-10-{[2-(2-methoxyphenyl)pyrimidin-4-yl]methoxy}-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylic acid;
(7R,20S)-19-chloro-1-(4-fluorophenyl)-10-{[2-(2-methoxyphenyl)pyrimidin-4-yl]methoxy}-18-methyl-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylic acid;
(7R,20S)-18-chloro-1-(4-fluorophenyl)-19-methyl-15-oxo-16-[2-(piperazin-1-yl)ethyl]-10-{[2-(3,3,3-trifluoropropoxy)pyrimidin-4-yl]methoxy}-7,8,15,16-tetrahydro-14H-17,20-etheno-13,9-(metheno)-6-oxa-2-thia-3,5,16-triazacyclooctadeca[1,2,3-cd]indene-7-carboxylic acid;
(7R,20S)-18-fluoro-1-(4-fluorophenyl)-19-methoxy-10-{[2-(2-methoxyphenyl)pyrimidin-4-yl]methoxy}-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylic acid;
(7R,20R)-18-chloro-1-(4-fluorophenyl)-19-methyl-16-[2-(4-methylpiperazin-1-yl)ethyl]-15-oxo-10-{[2-(3,3,3-trifluoropropoxy)pyrimidin-4-yl]methoxy}-7,8,15,16-tetrahydro-14H-17,20-etheno-13,9-(metheno)-6-oxa-2-thia-3,5,16-triazacyclooctadeca[1,2,3-cd]indene-7-carboxylic acid;
(7R,21S)-19-chloro-1-(4-fluorophenyl)-20-methyl-16-[2-(4-methylpiperazin-1-yl)ethyl]-15-oxo-10-{[2-(3,3,3-trifluoropropoxy)pyrimidin-4-yl]methoxy}-7,8,14,15,16,17-hexahydro-18,21-etheno-13,9-(metheno)-6-oxa-2-thia-3,5,16-triazacyclononadeca[1,2,3-cd]indene-7-carboxylic acid;
(7R,21S)-19-chloro-1-(4-fluorophenyl)-10-{[2-(2-methoxyphenyl)pyrimidin-4-yl]methoxy}-20-methyl-16-[2-(4-methylpiperazin-1-yl)ethyl]-15-oxo-7,8,14,15,16,17-hexahydro-18,21-etheno-13,9-(metheno)-6-oxa-2-thia-3,5,16-triazacyclononadeca[1,2,3-cd]indene-7-carboxylic acid;
(7R,21R)-19-chloro-1-(4-fluorophenyl)-10-{[2-(2-methoxyphenyl)pyrimidin-4-yl]methoxy}-20-methyl-16-[2-(4-methylpiperazin-1-yl)ethyl]-15-oxo-7,8,14,15,16,17-hexahydro-18,21-etheno-13,9-(metheno)-6-oxa-2-thia-3,5,16-triazacyclononadeca[1,2,3-cd]indene-7-carboxylic acid;
(7R,20S)-18-chloro-1-(4-fluorophenyl)-10-{[2-(2-methoxyphenyl)pyrimidin-4-yl]methoxy}-19-methyl-16-[2-(4-methylpiperazin-1-yl)ethyl]-15-oxo-7,8,15,16-tetrahydro-14H-17,20-etheno-13,9-(metheno)-6-oxa-2-thia-3,5,16-triazacyclooctadeca[1,2,3-cd]indene-7-carboxylic acid;
(7R)-18-chloro-1-(4-fluorophenyl)-10-{[2-(2-methoxyphenyl)pyrimidin-4-yl]methoxy}-19-methyl-15-oxo-7,8,15,16-tetrahydro-14H-17,20-etheno-13,9-(metheno)-6-oxa-2-thia-3,5,16-triazacyclooctadeca[1,2,3-cd]indene-7-carboxylic acid;
(7R,20S)-18-chloro-1-(4-fluorophenyl)-10-{[2-(2-methoxyphenyl)pyrimidin-4-yl]methoxy}-19-methyl-16-[3-(4-methylpiperazin-1-yl)propyl]-15-oxo-7,8,15,16-tetrahydro-14H-17,20-etheno-13,9-(metheno)-6-oxa-2-thia-3,5,16-triazacyclooctadeca[1,2,3-cd]indene-7-carboxylic acid;
(7R,21S)-19-chloro-1-(4-fluorophenyl)-20-methyl-17-[2-(4-methylpiperazin-1-yl)ethyl]-16-oxo-10-{[2-(3,3,3-trifluoropropoxy)pyrimidin-4-yl]methoxy}-7,8,16,17-tetrahydro-15H-18,21-etheno-13,9-(metheno)-6,14-dioxa-2-thia-3,5,17-triazacyclononadeca[1,2,3-cd]indene-7-carboxylic acid;
(7R,21S)-19-chloro-1-(4-fluorophenyl)-20-methyl-17-[2-(4-methylpiperazin-1-yl)ethyl]-10-{[2-(3,3,3-trifluoropropoxy)pyrimidin-4-yl]methoxy}-7,8,16,17-tetrahydro-15H-18,21-etheno-13,9-(metheno)-6,14-dioxa-2-thia-3,5,17-triazacyclononadeca[1,2,3-cd]indene-7-carboxylic acid;
(7R,21S)-19-chloro-1-(4-fluorophenyl)-20-methyl-10-{[2-(3,3,3-trifluoropropoxy)pyrimidin-4-yl]methoxy}-7,8,16,17-tetrahydro-15H-18,21-etheno-13,9-(metheno)-6,14-dioxa-2-thia-3,5,17-triazacyclononadeca[1,2,3-cd]indene-7-carboxylic acid;
(7R,21R)-19-chloro-1-(4-fluorophenyl)-20-methyl-10-{[2-(3,3,3-trifluoropropoxy)pyrimidin-4-yl]methoxy}-7,8,16,17-tetrahydro-15H-18,21-etheno-13,9-(metheno)-6,14-dioxa-2-thia-3,5,17-triazacyclononadeca[1,2,3-cd]indene-7-carboxylic acid;
(7R,20S)-18-chloro-1-(4-fluorophenyl)-10-{[2-(2-methoxyphenyl)pyrimidin-4-yl]methoxy}-19-methyl-15-[2-(morpholin-4-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylic acid;
[(2,2-dimethylpropanoyl)oxy]methyl (7R,21S)-19-chloro-1-(4-fluorophenyl)-10-{[2-(2-methoxyphenyl)pyrimidin-4-yl]methoxy}-20-methyl-16-[2-(4-methylpiperazin-1-yl)ethyl]-15-oxo-7,8,14,15,16,17-hexahydro-18,21-etheno-13,9-(metheno)-6-oxa-2-thia-3,5,16-triazacyclononadeca[1,2,3-cd]indene-7-carboxylate;
(7R,20S)-18-chloro-1-(4-fluorophenyl)-15-(2-methoxyethyl)-10-{[2-(2-methoxyphenyl)pyrimidin-4-yl]methoxy}-19-methyl-7,8,15,16-tetrahydro-14H-17,20-etheno-13,9-(metheno)-6-oxa-2-thia-3,5,15-triazacyclooctadeca[1,2,3-cd]indene-7-carboxylic acid;
(7R,20S)-18-chloro-15-[2-(4,4-difluoropiperidin-1-yl)ethyl]-1-(4-fluorophenyl)-10-{[2-(2-methoxyphenyl)pyrimidin-4-yl]methoxy}-19-methyl-7,8,15,16-tetrahydro-14H-17,20-etheno-13,9-(metheno)-6-oxa-2-thia-3,5,15-triazacyclooctadeca[1,2,3-cd]indene-7-carboxylic acid;
(7R,20S)-18-chloro-1-(4-fluorophenyl)-15-[2-(2-methoxyethoxy)ethyl]-10-{[2-(2-methoxyphenyl)pyrimidin-4-yl]methoxy}-19-methyl-7,8,15,16-tetrahydro-14H-17,20-etheno-13,9-(metheno)-6-oxa-2-thia-3,5,15-triazacyclooctadeca[1,2,3-cd]indene-7-carboxylic acid;
(7R,21S)-19-chloro-1-(4-fluorophenyl)-10-{[2-(2-methoxyphenyl)pyrimidin-4-yl]methoxy}-20-methyl-17-[2-(4-methylpiperazin-1-yl)ethyl]-16-oxo-7,8,14,15,16,17-hexahydro-18,21-etheno-13,9-(metheno)-6-oxa-2-thia-3,5,17-triazacyclononadeca[1,2,3-cd]indene-7-carboxylic acid;
(7R,21R)-19-chloro-1-(4-fluorophenyl)-10-{[2-(2-methoxyphenyl)pyrimidin-4-yl]methoxy}-20-methyl-17-[2-(4-methylpiperazin-1-yl)ethyl]-16-oxo-7,8,14,15,16,17-hexahydro-18,21-etheno-13,9-(metheno)-6-oxa-2-thia-3,5,17-triazacyclononadeca[1,2,3-cd]indene-7-carboxylic acid;
(5-methyl-2-oxo-2H-1,3-dioxol-4-yl)methyl (7S,21S)-19-chloro-1-(4-fluorophenyl)-10-{[2-(2-methoxyphenyl)pyrimidin-4-yl]methoxy}-20-methyl-16-[2-(4-methylpiperazin-1-yl)ethyl]-15-oxo-7,8,14,15,16,17-hexahydro-18,21-etheno-13,9-(metheno)-6-oxa-2-thia-3,5,16-triazacyclononadeca[1,2,3-cd]indene-7-carboxylate;
(5-methyl-2-oxo-2H-1,3-dioxol-4-yl)methyl (7R,21S)-19-chloro-1-(4-fluorophenyl)-10-{[2-(2-methoxyphenyl)pyrimidin-4-yl]methoxy}-20-methyl-16-[2-(4-methylpiperazin-1-yl)ethyl]-15-oxo-7,8,14,15,16,17-hexahydro-18,21-etheno-13,9-(metheno)-6-oxa-2-thia-3,5,16-triazacyclononadeca[1,2,3-cd]indene-7-carboxylate;
(7R,20S)-18-chloro-1-(4-fluorophenyl)-10-{[2-(2-methoxyphenyl)pyrimidin-4-yl]methoxy}-19-methyl-15-{[3-(morpholin-4-yl)oxetan-3-yl]methyl}-7,8,15,16-tetrahydro-14H-17,20-etheno-13,9-(metheno)-6-oxa-2-thia-3,5,15-triazacyclooctadeca[1,2,3-cd]indene-7-carboxylic acid;
(7R,20S)-18-chloro-1-(4-fluorophenyl)-10-{[2-(2-methoxyphenyl)pyrimidin-4-yl]methoxy}-19-methyl-15-[(oxan-4-yl)methyl]-7,8,15,16-tetrahydro-14H-17,20-etheno-13,9-(metheno)-6-oxa-2-thia-3,5,15-triazacyclooctadeca[1,2,3-cd]indene-7-carboxylic acid;
(7R,20S)-15-[2-(4-acetylpiperazin-1-yl)ethyl]-18-chloro-1-(4-fluorophenyl)-10-{[2-(2-methoxyphenyl)pyrimidin-4-yl]methoxy}-19-methyl-7,8,15,16-tetrahydro-14H-17,20-etheno-13,9-(metheno)-6-oxa-2-thia-3,5,15-triazacyclooctadeca[1,2,3-cd]indene-7-carboxylic acid;
(7R,20S)-18-chloro-1-(4-fluorophenyl)-15-{2-[(2-methoxyethyl)(methyl)amino]ethyl}-10-{[2-(2-methoxyphenyl)pyrimidin-4-yl]methoxy}-19-methyl-7,8,15,16-tetrahydro-14H-17,20-etheno-13,9-(metheno)-6-oxa-2-thia-3,5,15-triazacyclooctadeca[1,2,3-cd]indene-7-carboxylic acid;
(7R,20S)-18-chloro-1-(4-fluorophenyl)-N-hydroxy-10-{[2-(2-methoxyphenyl)pyrimidin-4-yl]methoxy}-19-methyl-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxamide;
(7R,20S)-18-chloro-1-(4-fluorophenyl)-15-[2-(4-hydroxypiperidin-1-yl)ethyl]-10-{[2-(2-methoxyphenyl)pyrimidin-4-yl]methoxy}-19-methyl-7,8,15,16-tetrahydro-14H-17,20-etheno-13,9-(metheno)-6-oxa-2-thia-3,5,15-triazacyclooctadeca[1,2,3-cd]indene-7-carboxylic acid;
(7R,21S)-19-chloro-1-(4-fluorophenyl)-20-methyl-15-oxo-16-{2-[4-(2,2,2-trifluoroethyl)piperazin-1-yl]ethyl}-10-{[2-(3,3,3-trifluoropropoxy)pyrimidin-4-yl]methoxy}-7,8,14,15,16,17-hexahydro-18,21-etheno-13,9-(metheno)-6-oxa-2-thia-3,5,16-triazacyclononadeca[1,2,3-cd]indene-7-carboxylic acid;
(7R,21R)-19-chloro-1-(4-fluorophenyl)-20-methyl-15-oxo-16-{2-[4-(2,2,2-trifluoroethyl)piperazin-1-yl]ethyl}-10-{[2-(3,3,3-trifluoropropoxy)pyrimidin-4-yl]methoxy}-7,8,14,15,16,17-hexahydro-18,21-etheno-13,9-(metheno)-6-oxa-2-thia-3,5,16-triazacyclononadeca[1,2,3-cd]indene-7-carboxylic acid;
(7R,20S)-18-chloro-15-[2-(dimethylamino)ethyl]-1-(4-fluorophenyl)-10-{[2-(2-methoxyphenyl)pyrimidin-4-yl]methoxy}-19-methyl-7,8,15,16-tetrahydro-14H-17,20-etheno-13,9-(metheno)-6-oxa-2-thia-3,5,15-triazacyclooctadeca[1,2,3-cd]indene-7-carboxylic acid;
(7R,20S)-18-chloro-1-(4-fluorophenyl)-15-(3-hydroxypropyl)-10-{[2-(2-methoxyphenyl)pyrimidin-4-yl]methoxy}-19-methyl-7,8,15,16-tetrahydro-14H-17,20-etheno-13,9-(metheno)-6-oxa-2-thia-3,5,15-triazacyclooctadeca[1,2,3-cd]indene-7-carboxylic acid;
(7R,20S)-18-chloro-1-(4-fluorophenyl)-10-{[2-(2-methoxyphenyl)pyrimidin-4-yl]methoxy}-15,19-dimethyl-7,8,15,16-tetrahydro-14H-17,20-etheno-13,9-(metheno)-6-oxa-2-thia-3,5,15-triazacyclooctadeca[1,2,3-cd]indene-7-carboxylic acid;
(7R,20S)-18-chloro-1-(4-fluorophenyl)-10-{[2-(2-methoxyphenyl)pyrimidin-4-yl]methoxy}-19-methyl-7,8,15,16-tetrahydro-14H-17,20-etheno-13,9-(metheno)-6-oxa-2-thia-3,5,15-triazacyclooctadeca[1,2,3-cd]indene-7-carboxylic acid;
(7R,20S)-18-chloro-15-[2-(4-cyclopropylpiperazin-1-yl)ethyl]-1-(4-fluorophenyl)-10-{[2-(2-methoxyphenyl)pyrimidin-4-yl]methoxy}-19-methyl-7,8,15,16-tetrahydro-14H-17,20-etheno-13,9-(metheno)-6-oxa-2-thia-3,5,15-triazacyclooctadeca[1,2,3-cd]indene-7-carboxylic acid;
(7R,20S)-18-chloro-10-{[2-(2-methoxyphenyl)pyrimidin-4-yl]methoxy}-19-methyl-15-[2-(4-methylpiperazin-1-yl)ethyl]-1-(prop-1-yn-1-yl)-7,8,15,16-tetrahydro-14H-17,20-etheno-13,9-(metheno)-6-oxa-2-thia-3,5,15-triazacyclooctadeca[1,2,3-cd]indene-7-carboxylic acid;
(7R,20S)-18-chloro-1-(4-fluorophenyl)-10-{[2-(2-methoxyphenyl)pyrimidin-4-yl]methoxy}-19-methyl-15-{2-[4-(2,2,2-trifluoroethyl)piperazin-1-yl]ethyl}-7,8,15,16-tetrahydro-14H-17,20-etheno-13,9-(metheno)-6-oxa-2-thia-3,5,15-triazacyclooctadeca[1,2,3-cd]indene-7-carboxylic acid;
(7R,20S)-ethyl 18-chloro-1-(4-fluorophenyl)-10-{[2-(2-methoxyphenyl)pyrimidin-4-yl]methoxy}-19-methyl-15-[2-(piperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylate;
(7R,20S)-18-chloro-1-(4-fluorophenyl)-15-[2-(3-hydroxypyrrolidin-1-yl)ethyl]-10-{[2-(2-methoxyphenyl)pyrimidin-4-yl]methoxy}-19-methyl-7,8,15,16-tetrahydro-14H-17,20-etheno-13,9-(metheno)-6-oxa-2-thia-3,5,15-triazacyclooctadeca[1,2,3-cd]indene-7-carboxylic acid;
(7R,20S)-18-chloro-15-[2-(4-hydroxypiperidin-1-yl)ethyl]-10-{[2-(2-methoxyphenyl)pyrimidin-4-yl]methoxy}-19-methyl-1-(prop-1-yn-1-yl)-7,8,15,16-tetrahydro-14H-17,20-etheno-13,9-(metheno)-6-oxa-2-thia-3,5,15-triazacyclooctadeca[1,2,3-cd]indene-7-carboxylic acid;
(7R,20R)-18-chloro-15-[2-(4-hydroxypiperidin-1-yl)ethyl]-10-{[2-(2-methoxyphenyl)pyrimidin-4-yl]methoxy}-19-methyl-1-(prop-1-yn-1-yl)-7,8,15,16-tetrahydro-14H-17,20-etheno-13,9-(metheno)-6-oxa-2-thia-3,5,15-triazacyclooctadeca[1,2,3-cd]indene-7-carboxylic acid;
(7R,20S)-18-chloro-1-(4-fluorophenyl)-10-{[2-(2-methoxyphenyl)pyrimidin-4-yl]methoxy}-19-methyl-15-[2-(1-methylpiperidin-4-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylic acid;
(7R,16R,21R)-19-chloro-1-(4-fluorophenyl)-10-{[2-(2-methoxyphenyl)pyrimidin-4-yl]methoxy}-20-methyl-16-[(4-methylpiperazin-1-yl)methyl]-7,8,15,16-tetrahydro-18,21-etheno-9,13-(metheno)-2,6,14,17-tetraoxa-3,5-diazacyclononadeca[1,2,3-cd]indene-7-carboxylic acid;
(7R,20S)-18-chloro-1-(4-fluorophenyl)-10-{[2-(2-methoxyphenyl)pyrimidin-4-yl]methoxy}-19-methyl-15-[3-(4-methylpiperazin-1-yl)propyl]-7,8,15,16-tetrahydro-14H-17,20-etheno-13,9-(metheno)-6-oxa-2-thia-3,5,15-triazacyclooctadeca[1,2,3-cd]indene-7-carboxylic acid;
(7R,21S)-19-chloro-16-[2-(4,4-difluoropiperidin-1-yl)ethyl]-1-(4-fluorophenyl)-20-methyl-15-oxo-10-{[2-(3,3,3-trifluoropropoxy)pyrimidin-4-yl]methoxy}-7,8,14,15,16,17-hexahydro-18,21-etheno-13,9-(metheno)-6-oxa-2-thia-3,5,16-triazacyclononadeca[1,2,3-cd]indene-7-carboxylic acid;
(7R,20S)-18-chloro-1-(4-fluorophenyl)-15-{3-[4-(2-hydroxyethyl)piperazin-1-yl]propyl}-10-{[2-(2-methoxyphenyl)pyrimidin-4-yl]methoxy}-19-methyl-7,8,15,16-tetrahydro-14H-17,20-etheno-13,9-(metheno)-6-oxa-2-thia-3,5,15-triazacyclooctadeca[1,2,3-cd]indene-7-carboxylic acid;
(7R,21R)-19-chloro-16-[2-(4,4-difluoropiperidin-1-yl)ethyl]-1-(4-fluorophenyl)-20-methyl-15-oxo-10-{[2-(3,3,3-trifluoropropoxy)pyrimidin-4-yl]methoxy}-7,8,14,15,16,17-hexahydro-18,21-etheno-13,9-(metheno)-6-oxa-2-thia-3,5,16-triazacyclononadeca[1,2,3-cd]indene-7-carboxylic acid;
(7R,21S)-19-chloro-1-(4-fluorophenyl)-16-{2-[4-(methanesulfonyl)piperazin-1-yl]ethyl}-20-methyl-15-oxo-10-{[2-(3,3,3-trifluoropropoxy)pyrimidin-4-yl]methoxy}-7,8,14,15,16,17-hexahydro-18,21-etheno-13,9-(metheno)-6-oxa-2-thia-3,5,16-triazacyclononadeca[1,2,3-cd]indene-7-carboxylic acid;
(7R,21S)-19-chloro-1-(4-fluorophenyl)-20-methyl-15-oxo-16-[2-(3-oxopiperazin-1-yl)ethyl]-10-{[2-(3,3,3-trifluoropropoxy)pyrimidin-4-yl]methoxy}-7,8,14,15,16,17-hexahydro-18,21-etheno-13,9-(metheno)-6-oxa-2-thia-3,5,16-triazacyclononadeca[1,2,3-cd]indene-7-carboxylic acid;
(7R,20S)-18-chloro-1-(4-fluorophenyl)-10-{[2-(2-methoxyphenyl)pyrimidin-4-yl]methoxy}-19-methyl-15-{2-[4-(methylamino)piperidin-1-yl]ethyl}-7,8,15,16-tetrahydro-14H-17,20-etheno-13,9-(metheno)-6-oxa-2-thia-3,5,15-triazacyclooctadeca[1,2,3-cd]indene-7-carboxylic acid;
(7R,20S)-18-chloro-15-{2-[4-(dimethylamino)piperidin-1-yl]ethyl}-1-(4-fluorophenyl)-10-{[2-(2-methoxyphenyl)pyrimidin-4-yl]methoxy}-19-methyl-7,8,15,16-tetrahydro-14H-17,20-etheno-13,9-(metheno)-6-oxa-2-thia-3,5,15-triazacyclooctadeca[1,2,3-cd]indene-7-carboxylic acid;
(7R,20S)-18-chloro-1-(4-fluorophenyl)-10-{[2-(2-methoxyphenyl)pyrimidin-4-yl]methoxy}-19-methyl-15-[2-(4-methyl-3-oxopiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylic acid;
ethyl (7R,21S)-19-chloro-1-(4-fluorophenyl)-10-{[2-(2-methoxyphenyl)pyrimidin-4-yl]methoxy}-20-methyl-15-oxo-16-[2-(piperazin-1-yl)ethyl]-7,8,14,15,16,17-hexahydro-18,21-etheno-13,9-(metheno)-6-oxa-2-thia-3,5,16-triazacyclononadeca[1,2,3-cd]indene-7-carboxylate;
(7S,16R,21S)-19-chloro-1-(4-fluorophenyl)-10-{[2-(2-methoxyphenyl)pyrimidin-4-yl]methoxy}-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,20S)-18-chloro-1-(4-fluorophenyl)-10-{[2-(2-methoxyphenyl)pyrimidin-4-yl]methoxy}-19-methyl-7,8-dihydro-14H,16H-17,20-etheno-13,9-(metheno)-6,15-dioxa-2-thia-3,5-diazacyclooctadeca[1,2,3-cd]indene-7-carboxylic acid;
(7R,20S)-18-chloro-1-(4-fluorophenyl)-10-{[2-(2-methoxyphenyl)pyrimidin-4-yl]methoxy}-19-methyl-15-[2-(piperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylic acid;
(7R,16R,21R)-19-chloro-1-(4-fluorophenyl)-10-{[2-(2-methoxyphenyl)pyrimidin-4-yl]methoxy}-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16S,21S)-19-chloro-1-(4-fluorophenyl)-20-methyl-16-[(4-methylpiperazin-1-yl)methyl]-10-{[2-(3,3,3-trifluoropropoxy)pyrimidin-4-yl]methoxy}-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,20S)-18-chloro-1-(4-fluorophenyl)-10-({2-[2-(2-methoxyethoxy)phenyl]pyrimidin-4-yl}methoxy)-19-methyl-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylic acid;
18-chloro-1-(4-fluorophenyl)-19-methyl-15-[2-(4-methylpiperazin-1-yl)ethyl]-10-{[2-(3-methylpyridin-4-yl)pyrimidin-4-yl]methoxy}-7,8,15,16-tetrahydro-14H-17,20-etheno-13,9-(metheno)-6-oxa-2-thia-3,5,15-triazacyclooctadeca[1,2,3-cd]indene-7-carboxylic acid;
(7R,21S)-19-chloro-1-(4-fluorophenyl)-10-{[2-(2-methoxyphenyl)pyrimidin-4-yl]methoxy}-20-methyl-15-oxo-16-[2-(piperazin-1-yl)ethyl]-7,8,14,15,16,17-hexahydro-18,21-etheno-13,9-(metheno)-6-oxa-2-thia-3,5,16-triazacyclononadeca[1,2,3-cd]indene-7-carboxylic acid;
(7R,16R,21S)-19-chloro-1-(4-fluorophenyl)-10-{[2-(2-methoxyphenyl)pyrimidin-4-yl]methoxy}-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid,
(7R,20R)-2,18-dichloro-1-(4-fluorophenyl)-10-{[2-(2-methoxyphenyl)pyrimidin-4-yl]methoxy}-19-methyl-15-[2-(4-methylpiperazin-1-yl)ethyl]-7,8,15,16-tetrahydro-14H-17,20-etheno-9,13-(metheno)-6-oxa-2a,5,15-triazacyclooctadeca[1,2,3-cd]indene-7-carboxylic acid;
(7R,20S)-10-[(1-butyl-1H-pyrazol-5-yl)methoxy]-18-chloro-1-(4-fluorophenyl)-19-methyl-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylic acid;
(7R,20S)-18-chloro-1-(4-fluorophenyl)-19-methyl-15-[2-(4-methylpiperazin-1-yl)ethyl]-10-{[2-(3,3,3-trifluoropropoxy)pyrimidin-4-yl]methoxy}-7,8,15,16-tetrahydro-14H-17,20-etheno-13,9-(metheno)-6-oxa-2-thia-3,5,15-triazacyclooctadeca[1,2,3-cd]indene-7-carboxylic acid;
(7R,20S)-2,18-dichloro-1-(4-fluorophenyl)-10-{[2-(2-methoxyphenyl)pyrimidin-4-yl]methoxy}-19-methyl-15-[2-(4-methylpiperazin-1-yl)ethyl]-7,8,15,16-tetrahydro-14H-17,20-etheno-9,13-(metheno)-6-oxa-2a,5,15-triazacyclooctadeca[1,2,3-cd]indene-7-carboxylic acid;
(7R,16R,21S)-19-chloro-1-(4-fluorophenyl)-10-{[2-(2-methoxyphenyl)pyrimidin-4-yl]methoxy}-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,20S)-18-chloro-1-(4-fluorophenyl)-10-{[2-(2-methoxyphenyl)pyrimidin-4-yl]methoxy}-19-methyl-15-[3-(4-methylpiperazin-1-yl)propanoyl]-7,8,15,16-tetrahydro-14H-17,20-etheno-13,9-(metheno)-6-oxa-2-thia-3,5,15-triazacyclooctadeca[1,2,3-cd]indene-7-carboxylic acid;
(7R,16R,21R)-2,19-dichloro-1-(4-fluorophenyl)-10-{[2-(2-methoxyphenyl)pyrimidin-4-yl]methoxy}-20-methyl-16-[(4-methylpiperazin-1-yl)methyl]-7,8,15,16-tetrahydro-18,21-etheno-9,13-(metheno)-6,14,17-trioxa-2a,5-diazacyclononadeca[1,2,3-cd]indene-7-carboxylic acid;
(7R,20S)-18-chloro-1-(4-fluorophenyl)-19-methyl-10-[(4-{3-[2-(4-methylpiperazin-1-yl)ethoxy]phenyl}pyrimidin-2-yl)methoxy]-7,8-dihydro-14H,16H-17,20-etheno-13,9-(metheno)-6,15-dioxa-2-thia-3,5-diazacyclooctadeca[1,2,3-cd]indene-7-carboxylic acid;
(7R,20S)-18-chloro-1-(4-fluorophenyl)-10-{[2-(3-methoxyphenyl)pyrimidin-4-yl]methoxy}-19-methyl-7,8-dihydro-14H,16H-17,20-etheno-13,9-(metheno)-6,15-dioxa-2-thia-3,5-diazacyclooctadeca[1,2,3-cd]indene-7-carboxylic acid;
(7R,20S)-22-chloro-1-(4-fluorophenyl)-21-methyl-10-[(2-{3-[2-(4-methylpiperazin-1-yl)ethoxy]phenyl}pyrimidin-4-yl)methoxy]-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylic acid;
(7R,21S)-19-chloro-1-(4-fluorophenyl)-10-{[2-(2-methoxyphenyl)pyrimidin-4-yl]methoxy}-20-methyl-7,8,15,16-tetrahydro-18,21-etheno-9,13-(metheno)-6,14,17-trioxa-2-thia-3,5-diazacyclononadeca[1,2,3-cd]indene-7-carboxylic acid;
(7R,21S)-23-chloro-1-(4-fluorophenyl)-10-{[2-(2-methoxyphenyl)pyrimidin-4-yl]methoxy}-22-methyl-7,8,16,17-tetrahydro-15H-18,21-etheno-13,9-(metheno)-6,14-dioxa-2-thia-3,5,17-triazacyclononadeca[1,2,3-cd]indene-7-carboxylic acid;
(7R,21S)-23-chloro-1-(4-fluorophenyl)-10-{[2-(2-methoxyphenyl)pyrimidin-4-yl]methoxy}-22-methyl-17-[2-(morpholin-4-yl)ethyl]-7,8,16,17-tetrahydro-15H-18,21-etheno-13,9-(metheno)-6,14-dioxa-2-thia-3,5,17-triazacyclononadeca[1,2,3-cd]indene-7-carboxylic acid;
(7R,16R,21S)-19-chloro-1-(4-fluorophenyl)-16-({4-[2-(methanesulfonyl)ethyl]piperazin-1-yl}methyl)-10-{[2-(2-methoxyphenyl)pyrimidin-4-yl]methoxy}-20-methyl-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,20S)-18-chloro-1-(4-fluorophenyl)-10-({2-[3-(2-methoxyethyl)oxetan-3-yl]pyrimidin-4-yl}methoxy)-19-methyl-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylic acid;
(7R,20S)-10-[(2-{(2S)-1-[(benzyloxy)carbonyl]pyrrolidin-2-yl}pyrimidin-4-yl)methoxy]-18-chloro-1-(4-fluorophenyl)-19-methyl-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylic acid;
(7R,20S)-18-chloro-1-(4-fluorophenyl)-19-methyl-15-[2-(4-methylpiperazin-1-yl)ethyl]-10-({2-[(2R)-oxolan-2-yl]pyrimidin-4-yl}methoxy)-7,8,15,16-tetrahydro-14H-17,20-etheno-13,9-(metheno)-6-oxa-2-thia-3,5,15-triazacyclooctadeca[1,2,3-cd]indene-7-carboxylic acid;
(7R,20S)-18-chloro-1-(4-fluorophenyl)-19-methyl-15-[2-(4-methylpiperazin-1-yl)ethyl]-10-({2-[(2S*)-oxolan-2-yl]pyrimidin-4-yl}methoxy)-7,8,15,16-tetrahydro-14H-17,20-etheno-13,9-(metheno)-6-oxa-2-thia-3,5,15-triazacyclooctadeca[1,2,3-cd]indene-7-carboxylic acid;
(7R,20S)-18-chloro-1-(4-fluorophenyl)-19-methyl-15-[2-(4-methylpiperazin-1-yl)ethyl]-10-({2-[(2S*)-pyrrolidin-2-yl]pyrimidin-4-yl}methoxy)-7,8,15,16-tetrahydro-14H-17,20-etheno-13,9-(metheno)-6-oxa-2-thia-3,5,15-triazacyclooctadeca[1,2,3-cd]indene-7-carboxylic acid;
(7R,16R)-19,23-dichloro-1-(4-fluorophenyl)-10-{[2-(2-methoxyphenyl)pyrimidin-4-yl]methoxy}-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic acid;
(7R,20S)-18-chloro-1-(4-fluorophenyl)-19-methyl-15-[2-(4-methylpiperazin-1-yl)ethyl]-10-({2-[(1S,4S)-2-oxa-5-azabicyclo[2.2.1]heptan-5-yl]pyrimidin-4-yl}methoxy)-7,8,15,16-tetrahydro-14H-17,20-etheno-13,9-(metheno)-6-oxa-2-thia-3,5,15-triazacyclooctadeca[1,2,3-cd]indene-7-carboxylic acid;
(7R,20S)-18-chloro-1-(4-fluorophenyl)-19-methyl-10-({2-[(3R)-3-methylmorpholin-4-yl]pyrimidin-4-yl}methoxy)-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylic acid;
(7R,16R,21S)-19-chloro-1-(4-fluorophenyl)-16-{[(2-methoxyethyl)(methyl)amino]methyl}-10-{[2-(2-methoxyphenyl)pyrimidin-4-yl]methoxy}-20-methyl-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R,21S)-19-chloro-1-(4-fluorophenyl)-16-({(3R)-3-[(methanesulfonyl)methyl]-4-methylpiperazin-1-yl}methyl)-10-{[2-(2-methoxyphenyl)pyrimidin-4-yl]methoxy}-20-methyl-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R,21S)-19-chloro-1-(4-fluorophenyl)-16-({(3R)-3-[(methanesulfonyl)methyl]piperazin-1-yl}methyl)-10-{[2-(2-methoxyphenyl)pyrimidin-4-yl]methoxy}-20-methyl-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R,21S)-19-chloro-16-[(1,1-dioxo-1λ⁶-thiomorpholin-4-yl)methyl]-1-(4-fluorophenyl)-10-{[2-(2-methoxyphenyl)pyrimidin-4-yl]methoxy}-20-methyl-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R,21S)-19-chloro-1-(4-fluorophenyl)-10-{[2-(2-methoxyphenyl)pyrimidin-4-yl]methoxy}-20-methyl-16-[(4-methyl-3-oxopiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,20S)-18-chloro-1-(4-fluorophenyl)-19-methyl-15-[2-(4-methylpiperazin-1-yl)ethyl]-10-({2-[(1R,5S)-3-oxa-8-azabicyclo[3.2.1]octan-8-yl]pyrimidin-4-yl}methoxy)-7,8,15,16-tetrahydro-14H-17,20-etheno-13,9-(metheno)-6-oxa-2-thia-3,5,15-triazacyclooctadeca[1,2,3-cd]indene-7-carboxylic acid;
(7R,20S)-18-chloro-10-{[2-(2,6-dioxa-9-azaspiro[4.5]decan-9-yl)pyrimidin-4-yl]methoxy}-1-(4-fluorophenyl)-19-methyl-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylic acid;
(7R,20S)-10-{[2-(bicyclo[1.1.1]pentan-1-yl)pyrimidin-4-yl]methoxy}-18-chloro-1-(4-fluorophenyl)-19-methyl-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylic acid;
(7R,20S)-18-chloro-1-(4-fluorophenyl)-19-methyl-10-({2-[(4-methyloxan-4-yl)methyl]pyrimidin-4-yl}methoxy)-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylic acid;
(7R,20S)-18-chloro-10-{[2-(2-cyanophenyl)pyrimidin-4-yl]methoxy}-1-(4-fluorophenyl)-19-methyl-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylic acid;
(7R,20S)-18-chloro-10-({2-[2-(dimethylphosphoryl)phenyl]pyrimidin-4-yl}methoxy)-1-(4-fluorophenyl)-19-methyl-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylic acid;
(7R,16R,21S)-19-chloro-1-(4-fluorophenyl)-16-({[2-(methanesulfonyl)ethyl](methyl)amino}methyl)-10-{[2-(2-methoxyphenyl)pyrimidin-4-yl]methoxy}-20-methyl-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R,21S)-19-chloro-16-[(dimethylamino)methyl]-1-(4-fluorophenyl)-10-{[2-(2-methoxyphenyl)pyrimidin-4-yl]methoxy}-20-methyl-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R,21S)-19-chloro-10-{(R)-fluoro[2-(2-methoxyphenyl)pyrimidin-4-yl]methoxy}-1-(4-fluorophenyl)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R,21S)-19-chloro-10-{(S)-fluoro[2-(2-methoxyphenyl)pyrimidin-4-yl]methoxy}-1-(4-fluorophenyl)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R,21S)-2,19-dichloro-1-(4-fluorophenyl)-10-{[2-(2-methoxyphenyl)pyrimidin-4-yl]methoxy}-20-methyl-16-[(4-methylpiperazin-1-yl)methyl]-7,8,15,16-tetrahydro-18,21-etheno-9,13-(metheno)-6,14,17-trioxa-2a,5-diazacyclononadeca[1,2,3-cd]indene-7-carboxylic acid;
(7S,16R,21R)-2,19-dichloro-1-(4-fluorophenyl)-10-{[2-(2-methoxyphenyl)pyrimidin-4-yl]methoxy}-20-methyl-16-[(4-methylpiperazin-1-yl)methyl]-7,8,15,16-tetrahydro-18,21-etheno-9,13-(metheno)-6,14,17-trioxa-2a,5-diazacyclononadeca[1,2,3-cd]indene-7-carboxylic acid;
(7R,16R,21S)-19-chloro-1-cyclopropyl-10-{[2-(2-methoxyphenyl)pyrimidin-4-yl]methoxy}-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7S,16R,21S)-19-chloro-1-cyclopropyl-10-{[2-(2-methoxyphenyl)pyrimidin-4-yl]methoxy}-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R,21R)-23-chloro-1-cyclopropyl-10-{[2-(2-methoxyphenyl)pyrimidin-4-yl]methoxy}-22-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19-chloro-1-(4-fluorophenyl)-10-{[2-(2-methoxyphenyl)pyrimidin-4-yl]methoxy}-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic acid;
(7R,16R)-23-chloro-1-(4-fluorophenyl)-10-{[2-(2-methoxyphenyl)pyrimidin-4-yl]methoxy}-16-[(4-methylpiperazin-1-yl)methyl]-7,8,15,16-tetrahydro-18,21-etheno-9,13-(metheno)-2,6,14,17-tetraoxa-3,5-diazacyclononadeca[1,2,3-cd]indene-7-carboxylic acid;
(7R,16R,21S)-19-chloro-16-[(4,4-difluoropiperidin-1-yl)methyl]-1-(4-fluorophenyl)-10-{[2-(2-methoxyphenyl)pyrimidin-4-yl]methoxy}-20-methyl-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R,21S)-19-chloro-1-(4-fluorophenyl)-10-{[2-(2-methoxyphenyl)pyrimidin-4-yl]methoxy}-20-methyl-16-({methyl[2-(morpholin-4-yl)ethyl]amino}methyl)-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R,21S)-19-chloro-1-(4-fluorophenyl)-10-{[2-(2-methoxyphenyl)pyrimidin-4-yl]methoxy}-20-methyl-16-{[(3R,5S)-3,4,5-trimethylpiperazin-1-yl]methyl}-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-19,23-dichloro-1-(4-fluorophenyl)-10-{[2-(2-methoxyphenyl)pyrimidin-4-yl]methoxy}-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7S,16R)-19,23-dichloro-1-(4-fluorophenyl)-10-{[2-(2-methoxyphenyl)pyrimidin-4-yl]methoxy}-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R,21S)-19-chloro-1-(4-fluorophenyl)-10-{[2-(2-methoxyphenyl)pyrimidin-4-yl]methoxy}-20-methyl-16-{[4-(2,2,2-trifluoroethyl)piperazin-1-yl]methyl}-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R,21S)-16-{[bis(2-methoxyethyl)amino]methyl}-19-chloro-1-(4-fluorophenyl)-10-{[2-(2-methoxyphenyl)pyrimidin-4-yl]methoxy}-20-methyl-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R,21S)-23-chloro-10-{[2-(2-methoxyphenyl)pyrimidin-4-yl]methoxy}-22-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R)-2,19,23-trichloro-1-(4-fluorophenyl)-10-{[2-(2-methoxyphenyl)pyrimidin-4-yl]methoxy}-16-[(4-methylpiperazin-1-yl)methyl]-7,8,15,16-tetrahydro-18,21-etheno-9,13-(metheno)-6,14,17-trioxa-2a,5-diazacyclononadeca[1,2,3-cd]indene-7-carboxylic acid;
(7R,16R,21S)-19-chloro-10-{[2-(2-cyanophenyl)pyrimidin-4-yl]methoxy}-1-(4-fluorophenyl)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,20R)-18-chloro-10-{[2-(3-fluoro-2-methoxyphenyl)pyrimidin-4-yl]methoxy}-1-(4-fluorophenyl)-19-methyl-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylic acid;
(7R,20S)-18-chloro-10-{[2-(5-fluoro-2-methoxyphenyl)pyrimidin-4-yl]methoxy}-1-(4-fluorophenyl)-19-methyl-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylic acid;
(7R,20S)-18-chloro-1-(4-fluorophenyl)-10-{[2-(4-hydroxyphenyl)pyrimidin-4-yl]methoxy}-19-methyl-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylic acid;
(7R,16R)-1-(4-fluorophenyl)-10-{[2-(2-methoxyphenyl)pyrimidin-4-yl]methoxy}-16-[(4-methylpiperazin-1-yl)methyl]-7,8,15,16-tetrahydro-18,21-etheno-9,13-(metheno)-2,6,14,17-tetraoxa-3,5-diazacyclononadeca[1,2,3-cd]indene-7-carboxylic acid;
(7R,16R)-19,23-dichloro-1-(4-fluorophenyl)-10-{[2-(2-methoxyphenyl)pyrimidin-4-yl]methoxy}-16-[(4-methylpiperazin-1-yl)methyl]-7,8,15,16-tetrahydro-14H-18,21-etheno-9,13-(metheno)-6,17-dioxa-2-thia-3,5-diazacyclononadeca[1,2,3-cd]indene-7-carboxylic acid;
(7S,16R)-19,23-dichloro-1-(4-fluorophenyl)-10-{[2-(2-methoxyphenyl)pyrimidin-4-yl]methoxy}-16-[(4-methylpiperazin-1-yl)methyl]-7,8,15,16-tetrahydro-14H-18,21-etheno-9,13-(metheno)-6,17-dioxa-2-thia-3,5-diazacyclononadeca[1,2,3-cd]indene-7-carboxylic acid;
(7R,16R)-19,23-dichloro-1-(4-fluorophenyl)-10-{[2-(2-methoxyphenyl)pyrimidin-4-yl]methoxy}-16-[(4-methylpiperazin-1-yl)methyl]-7,8,15,16-tetrahydro-18,21-etheno-9,13-(metheno)-2,6,14,17-tetraoxa-3,5-diazacyclononadeca[1,2,3-cd]indene-7-carboxylic acid;
(7R,20S)-18-chloro-1-(4-fluorophenyl)-10-({2-[2-(methanesulfonyl)phenyl]pyrimidin-4-yl}methoxy)-19-methyl-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylic acid;
(7R,20S)-18-chloro-1-(4-fluorophenyl)-19-methyl-15-[2-(4-methylpiperazin-1-yl)ethyl]-10-({2-[(3R)-oxolan-3-yl]pyrimidin-4-yl}methoxy)-7,8,15,16-tetrahydro-14H-17,20-etheno-13,9-(metheno)-6-oxa-2-thia-3,5,15-triazacyclooctadeca[1,2,3-cd]indene-7-carboxylic acid;
(7R,20S)-18-chloro-1-(4-fluorophenyl)-19-methyl-15-[2-(4-methylpiperazin-1-yl)ethyl]-10-({2-[(3S)-oxolan-3-yl]pyrimidin-4-yl}methoxy)-7,8,15,16-tetrahydro-14H-17,20-etheno-13,9-(metheno)-6-oxa-2-thia-3,5,15-triazacyclooctadeca[1,2,3-cd]indene-7-carboxylic acid;
(7R,16R,21S)-19-chloro-16-{[(3R)-3,4-dimethylpiperazin-1-yl]methyl}-1-(4-fluorophenyl)-10-{[2-(2-methoxyphenyl)pyrimidin-4-yl]methoxy}-20-methyl-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16S,21S)-19-chloro-1-(4-fluorophenyl)-10-{[2-(2-methoxyphenyl)pyrimidin-4-yl]methoxy}-20-methyl-16-[(4-methylpiperazin-1-yl)methyl]-7,8,15,16-tetrahydro-14H-18,21-etheno-9,13-(metheno)-6,17-dioxa-2-thia-3,5-diazacyclononadeca[1,2,3-cd]indene-7-carboxylic acid;
(7S,16S,21S)-19-chloro-1-(4-fluorophenyl)-10-{[2-(2-methoxyphenyl)pyrimidin-4-yl]methoxy}-20-methyl-16-[(4-methylpiperazin-1-yl)methyl]-7,8,15,16-tetrahydro-14H-18,21-etheno-9,13-(metheno)-6,17-dioxa-2-thia-3,5-diazacyclononadeca[1,2,3-cd]indene-7-carboxylic acid;
(7R,16R,21S)-10-(benzyloxy)-19-chloro-1-(4-fluorophenyl)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7S,16R)-19,23-dichloro-1-(4-fluorophenyl)-10-{[2-(2-methoxyphenyl)pyrimidin-4-yl]methoxy}-16-[(4-methylpiperazin-1-yl)methyl]-7,8,15,16-tetrahydro-18,21-etheno-9,13-(metheno)-2,6,14,17-tetraoxa-3,5-diazacyclononadeca[1,2,3-cd]indene-7-carboxylic acid;
(7R,16R)-19-chloro-1-cyclobutyl-10-{[2-(2-methoxyphenyl)pyrimidin-4-yl]methoxy}-20-methyl-16-[(4-methylpiperazin-1-yl)methyl]-7,8,15,16-tetrahydro-18,21-etheno-13,9-(metheno)-2,6,14,17-tetraoxa-3,5-diazacyclononadeca[1,2,3-cd]indene-7-carboxylic acid;
(7R,16R,21S)-19-chloro-10-({2-[2-(difluoromethoxy)phenyl]pyrimidin-4-yl}methoxy)-1-(4-fluorophenyl)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R,21S)-19-chloro-1-(4-fluorophenyl)-10-({2-[2-(methoxymethyl)phenyl]pyrimidin-4-yl}methoxy)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,20S)-18-chloro-1-(4-fluorophenyl)-19-methyl-15-[2-(4-methylpiperazin-1-yl)ethyl]-10-({2-[(2R)-oxan-2-yl]pyrimidin-4-yl}methoxy)-7,8,15,16-tetrahydro-14H-17,20-etheno-13,9-(metheno)-6-oxa-2-thia-3,5,15-triazacyclooctadeca[1,2,3-cd]indene-7-carboxylic acid;
(7R,20S)-18-chloro-1-(4-fluorophenyl)-19-methyl-15-[2-(4-methylpiperazin-1-yl)ethyl]-10-({2-[(2S)-oxan-2-yl]pyrimidin-4-yl}methoxy)-7,8,15,16-tetrahydro-14H-17,20-etheno-13,9-(metheno)-6-oxa-2-thia-3,5,15-triazacyclooctadeca[1,2,3-cd]indene-7-carboxylic acid;
(7R,15S,21S)-19-chloro-1-(4-fluorophenyl)-10-{[2-(2-methoxyphenyl)pyrimidin-4-yl]methoxy}-20-methyl-15-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R,21S)-19-chloro-10-{[2-(5-fluoro-2-methoxyphenyl)pyrimidin-4-yl]methoxy}-1-(4-fluorophenyl)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R,21S)-19-chloro-1-(4-fluorophenyl)-20-methyl-16-[(4-methylpiperazin-1-yl)methyl]-10-({2-[(2S)-oxolan-2-yl]pyrimidin-4-yl}methoxy)-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R,21S)-19-chloro-1-(4-fluorophenyl)-10-({2-[2-(methanesulfonyl)phenyl]pyrimidin-4-yl}methoxy)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R,21S)-19-chloro-1-(4-fluorophenyl)-20-methyl-16-[(4-methylpiperazin-1-yl)methyl]-10-({2-[(2S)-oxan-2-yl]pyrimidin-4-yl}methoxy)-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R,21S)-19-chloro-1-(4-fluorophenyl)-10-{[2-(2-hydroxyphenyl)pyrimidin-4-yl]methoxy}-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R,21S)-19-chloro-1-(4-fluorophenyl)-10-({2-[4-(hydroxymethyl)phenyl]pyrimidin-4-yl}methoxy)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R,21S)-19-chloro-1-(4-fluorophenyl)-10-{[2-(4-hydroxyphenyl)pyrimidin-4-yl]methoxy}-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid;
(7R,16R,21S)-19-chloro-1-(4-fluorophenyl)-10-({2-[2-(hydroxymethyl)phenyl]pyrimidin-4-yl}methoxy)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid; and
   pharmaceutically acceptable salts thereof.

### Formula (II)

One embodiment pertains to compounds of Formula (IIa), (IIb), (IIc), (IId), or pharmaceutically acceptable salts thereof,

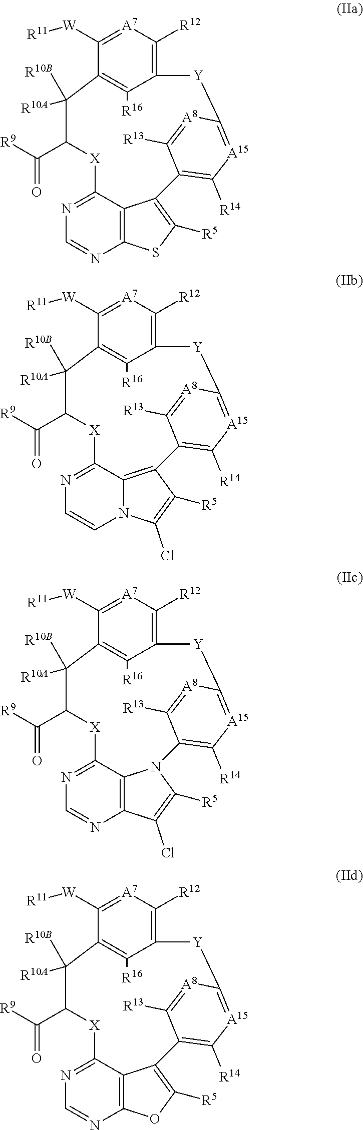

wherein A⁷, A⁸, A¹⁵, R⁵, R⁹, R^{10A}, R^{10B}, R¹¹, R¹², R¹³, R¹⁴, R¹⁶, W, X, and Y are as described in embodiments of Formula (I) herein.

### Formula (III)

One embodiment pertains to compounds of Formula (IIIa), (IIIb), (IIIc), (IIId), or pharmaceutically acceptable salts thereof,

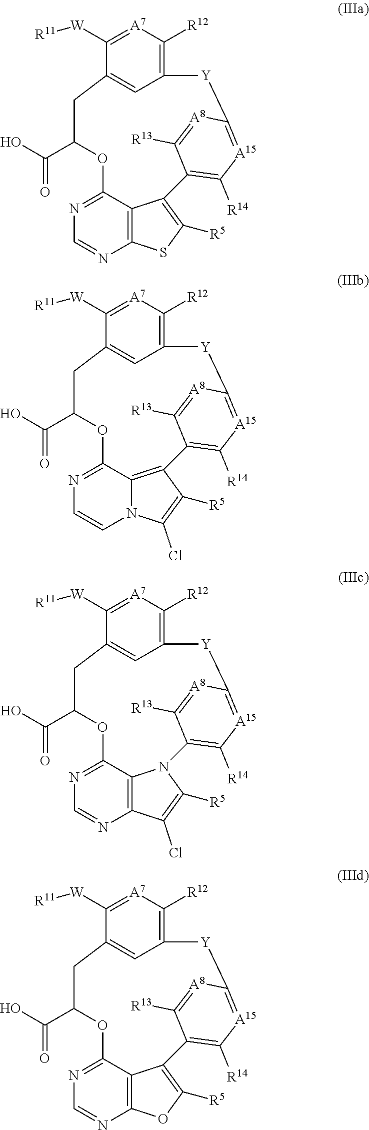

wherein A⁸, A¹⁵, R⁵, R¹¹, R¹³, R¹⁴, W, and Y are as described in embodiments of Formula (I) herein.

### Formula (IV)

One embodiment pertains to compounds of Formula (IVa), (IVb), (IVc), (IVd), or pharmaceutically acceptable salts thereof,

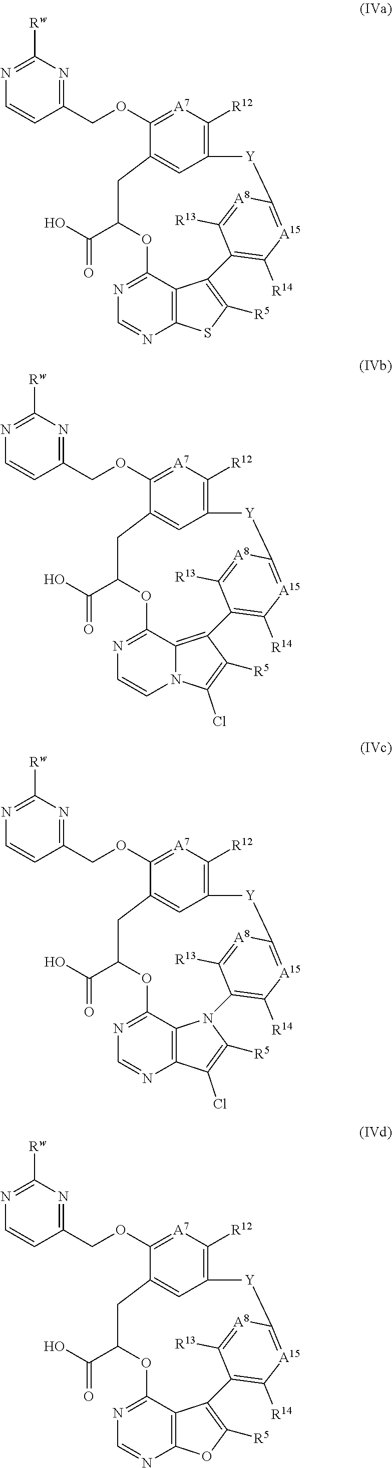

wherein A⁸, A¹⁵, R⁵, R¹³, R¹⁴, R^{w}, and Y are as described in embodiments of Formula (I) herein.

One embodiment pertains to compounds of Formula (IVa), (IVb), (IVc), and (IVd) wherein R^{w} is tetrahydrofuranyl, tetrahydropyranyl, or phenyl, optionally substituted with one R^{y}.

One embodiment pertains to compounds of Formula (IVa), (IVb), (IVc), and (IVd) wherein R^{w} is tetrahydrofuranyl, tetrahydropyranyl, or phenyl, optionally substituted with one OCH₃.

One embodiment pertains to compounds of Formula (IVa), (IVb), (IVc), and (IVd) wherein R^{w} is tetrahydrofuranyl, tetrahydropyranyl, or phenyl, optionally substituted with one OCH₃; and R^{s} is 4-fluorophenyl or cyclopropyl.

### Formula (V)

One embodiment pertains to compounds of Formula (Va), (Vb), (Vc), (Vd), or pharmaceutically acceptable salts thereof,

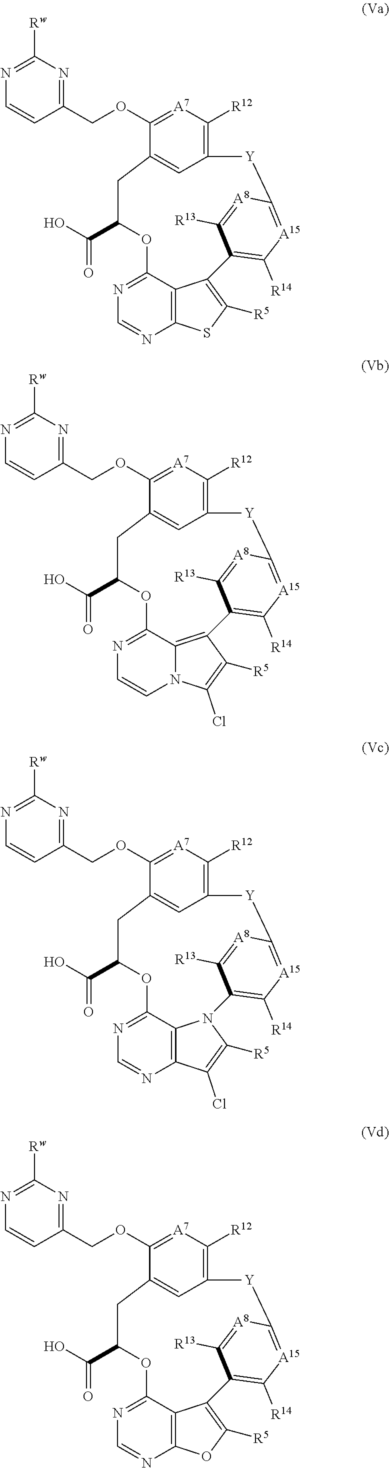

wherein A⁸, A¹⁵, R⁵, R¹³, R¹⁴, R^{w}, and Y are as described in embodiments of Formula (I) herein.

One embodiment pertains to compounds of Formula (Va), (Vb), (Vc), and (Vd) wherein R^{w} is tetrahydrofuranyl, tetrahydropyranyl, or phenyl, optionally substituted with one R^{y}.

One embodiment pertains to compounds of Formula (Va), (Vb), (Vc), and (Vd) wherein R^{w} is tetrahydrofuranyl, tetrahydropyranyl, or phenyl, optionally substituted with one OCH₃.

One embodiment pertains to compounds of Formula (Va), (Vb), (Vc), and (Vd) wherein R^{w} is tetrahydrofuranyl, tetrahydropyranyl, or phenyl, optionally substituted with one OCH₃; and R⁵ is 4-fluorophenyl or cyclopropyl.

Compound names are assigned by using Name 2016.1.1 (File Version N30E41, Build 86668) or Name 2017.2.1 (File Version N40E41, Build 96719) naming algorithm by Advanced Chemical Development or Struct=Name naming algorithm as part of CHEMDRAW® ULTRA v. 12.0.2.1076 or Professional Version 15.0.0.106.

Compounds according to the present disclosure may exist as atropisomers, resulting from hindered rotation about a single bond, when energy differences due to steric strain or other contributors create a barrier to rotation that is high enough to allow for isolation of individual conformers. See, e.g., Bringmann, G. et al., Atroposelective Synthesis of Axially Chiral Biaryl Compounds. Angew. Chem., Int. Ed., 2005, 44: 5384-5428. In some instances, the barrier of rotation is high enough that the different atropisomers may be separated and isolated, such as by chromatography on a chiral stationary phase. It is to be understood that the stereochemistry of the atropisomers is included in the compound names only when compounds are assayed as being pure (at least 95%) or are predominantly (at least 80%) one isomer. Where there is no atropisomer stereochemistry noted for a compound, then it is to be understood that either the stereochemistry is undetermined, or it was determined to be a near-equal mixture of atropisomers. In addition, where there is a discrepancy between the name of the compound and the structure found in Table 1, the structure depicted in Table 1 shall prevail.

Compounds of the present disclosure may exist as stereoisomers wherein asymmetric or chiral centers are present. These stereoisomers are “R” or “S” depending on the configuration of substituents around the chiral carbon atom. The terms “R” and “S” used herein are configurations as defined in IUPAC 1974 Recommendations for Section E, Fundamental Stereochemistry, in Pure Appl. Chem., 1976, 45: 13-30. The present disclosure contemplates various stereoisomers and mixtures thereof and these are specifically included within the scope of this disclosure. Stereoisomers include enantiomers and diastereomers, and mixtures of enantiomers or diastereomers. Individual stereoisomers of compounds of the present disclosure may be prepared synthetically from commercially available starting materials which contain asymmetric or chiral centers or by preparation of racemic mixtures followed by methods of resolution well-known to those of ordinary skill in the art. These methods of resolution are exemplified by (1) attachment of a mixture of enantiomers to a chiral auxiliary, separation of the resulting mixture of diastereomers by precipitation or chromatography and optional liberation of the optically pure product from the auxiliary as described in Furniss, Hannaford, Smith, and Tatchell, “Vogel's Textbook of Practical Organic Chemistry”, 5th edition (1989), Longman Scientific & Technical, Essex CM20 2JE, England, or (2) direct separation of the mixture of optical enantiomers on chiral chromatographic columns or (3) fractional recrystallization methods. It is to be understood that an asterisk (*) at a particular stereocenter in a structure of a chiral compound, indicates an arbitrary assignment of stereochemical configuration at that stereocenter. Moreover, an asterisk (*) following a stereochemical descriptor in the name of such a compound designates an arbitrary assignment of stereochemical configuration at that stereocenter.

Compounds of the present disclosure may exist as cis or trans isomers, wherein substituents on a ring may attached in such a manner that they are on the same side of the ring (cis) relative to each other, or on opposite sides of the ring relative to each other (trans). For example, cyclobutane may be present in the cis or trans configuration, and may be present as a single isomer or a mixture of the cis and trans isomers. Individual cis or trans isomers of compounds of the present disclosure may be prepared synthetically from commercially available starting materials using selective organic transformations, or prepared in single isomeric form by purification of mixtures of the cis and trans isomers. Such methods are well-known to those of ordinary skill in the art, and may include separation of isomers by recrystallization or chromatography.

It should be understood that the compounds of the present disclosure may possess tautomeric forms, as well as geometric isomers, and that these also constitute an aspect of the present disclosure.

The present disclosure includes all pharmaceutically acceptable isotopically-labeled compounds of Formula (I) wherein one or more atoms are replaced by atoms having the same atomic number, but an atomic mass or mass number different from the atomic mass or mass number which predominates in nature. Examples of isotopes suitable for inclusion in the compounds of the disclosure include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S. Certain isotopically-labeled compounds of Formula (I), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection. Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances. Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of Formula (I) may generally be prepared by conventional techniques known to those skilled in the art or by processes analogous to those described in the accompanying Examples using an appropriate isotopically-labeled reagents in place of the non-labeled reagent previously employed.

Thus, the Formula drawings within this specification can represent only one of the possible tautomeric, geometric, or stereoisomeric forms. It is to be understood that the present disclosure encompasses any tautomeric, geometric, or stereoisomeric form, and mixtures thereof, and is not to be limited merely to any one tautomeric, geometric, or stereoisomeric form utilized within the Formula drawings.

Exemplary compounds of Formula (I) include, but are not limited to, the compounds shown in Table 1 below. It is to be understood that when there is a discrepancy between the name of the compound found herein and the structure found in Table 1, the structure in Table 1 shall prevail. In addition, it is to be understood that an asterisk (*), at a particular stereocenter in a structure, indicates an arbitrary assignment of stereochemical configuration at that stereocenter.

TABLE 1

| EXAMPLE | STRUCTURE |
|---|---|
| 1 | 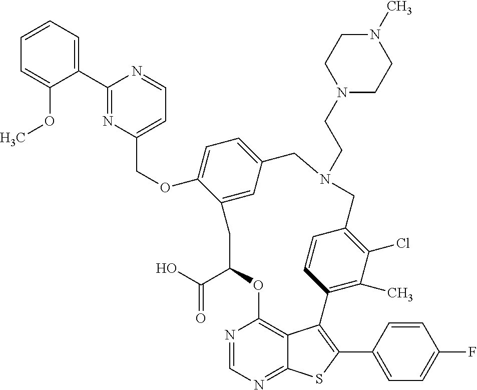 |
| 2 | 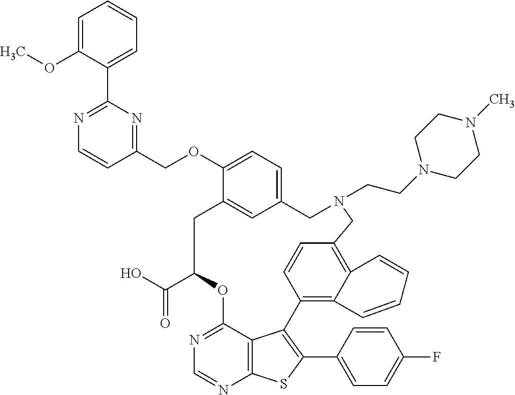 |
| 3 | 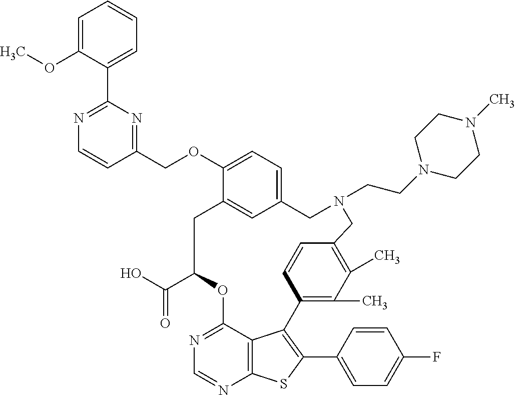 |
| 4 | 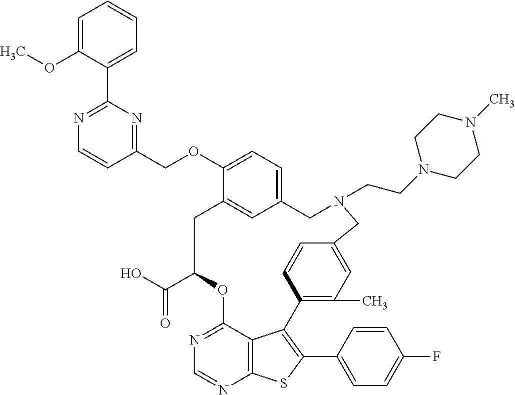 |
| 5 | 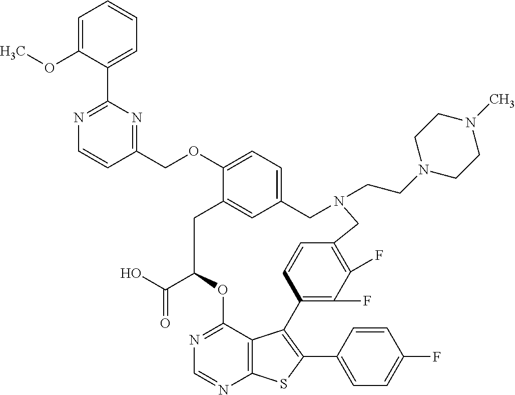 |
| 6 | 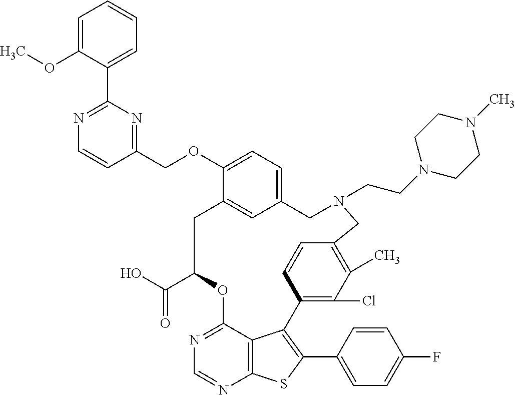 |
| 7 | 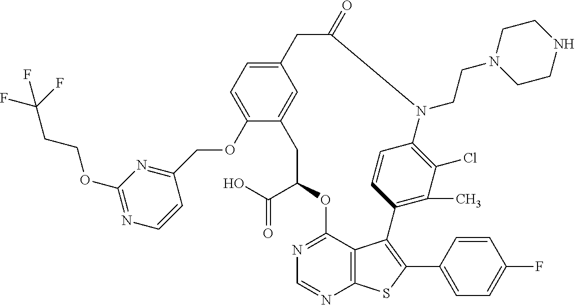 |
| 8 | 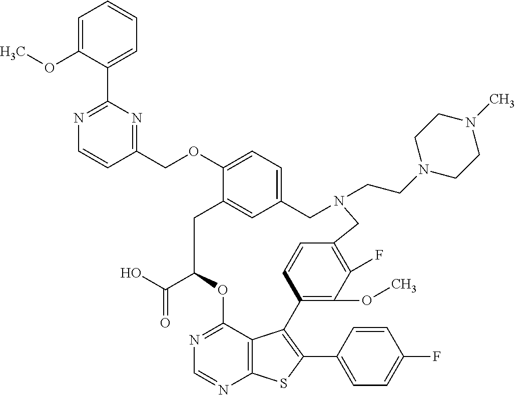 |
| 9 | 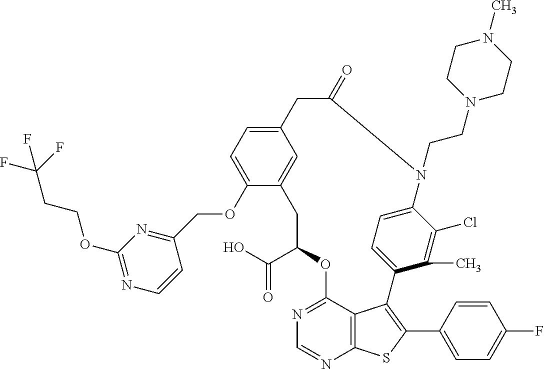 |
| 10 | 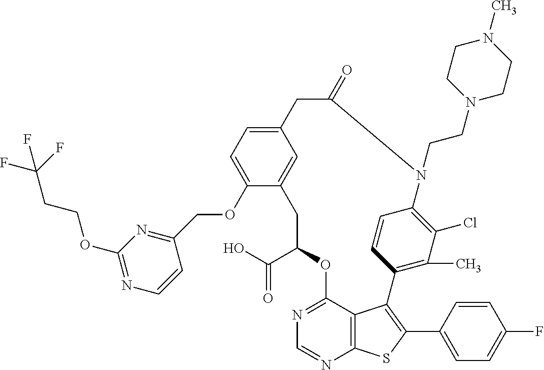 |
| 11 | 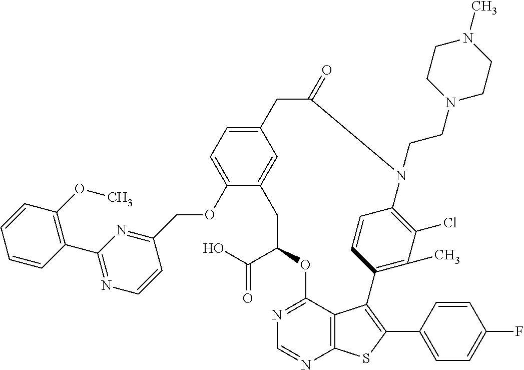 |
| 12 | 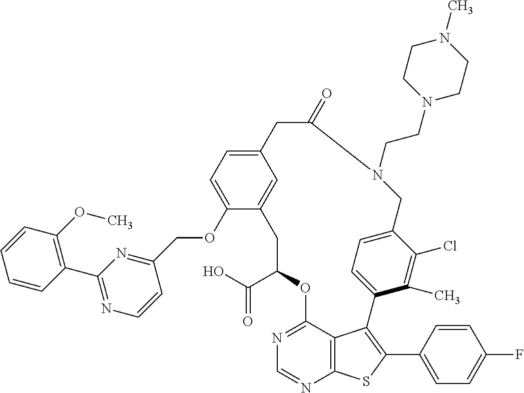 |
| 13 | 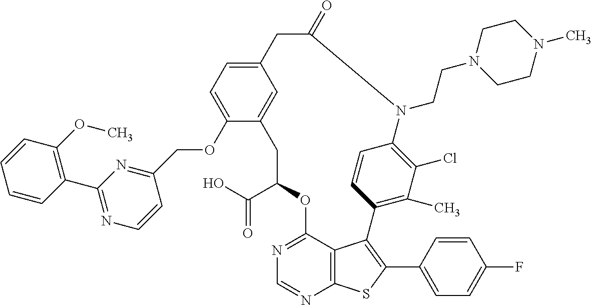 |
| 14 | 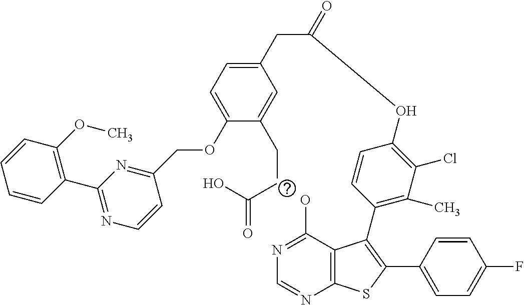 [SMILES: COc1ccccc1-c1nccc(COc2ccc(CC(=O)Oc3ccc(-c4c(-c5ccc(F)cc5)sc5ncnc(O)c45)c(C)c3Cl)cc2CCC(=O)O)n1] |
| 15 | 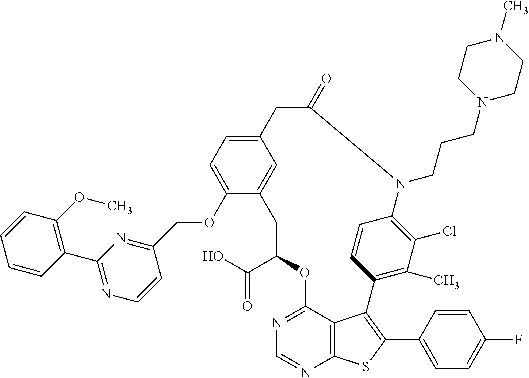 |
| 16 | 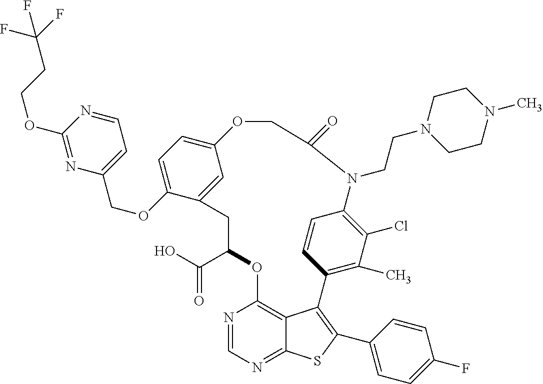 |
| 17 | 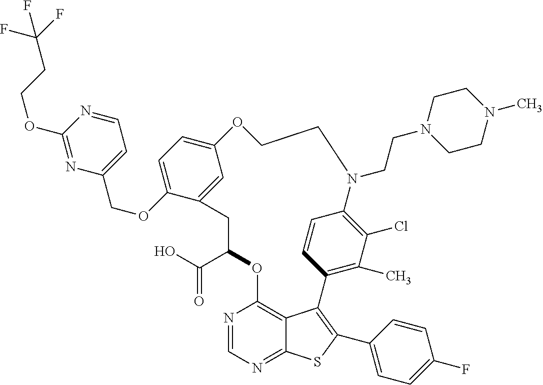 |
| 18 | 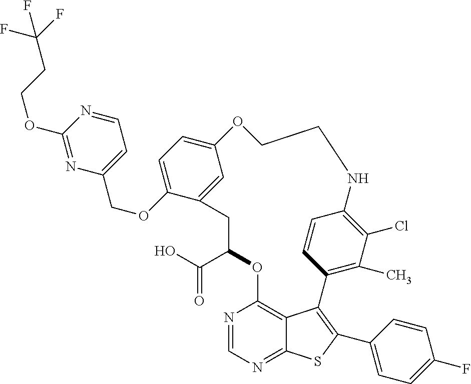 |
| 19 | 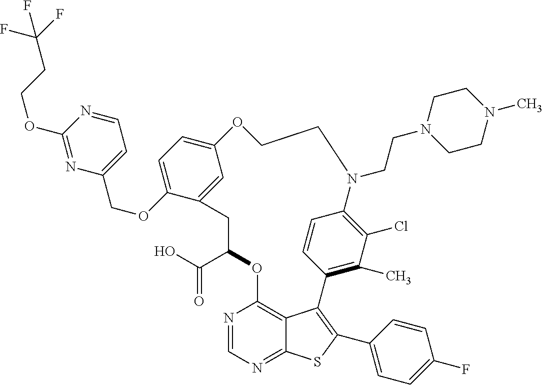 [SMILES: Cc1c2ccc(c1Cl)N(CCN1CCN(C)CC1)CCOc1ccc(OCc3ccnc(OCCC(F)(F)F)n3)c(c1)C[C@H](C(=O)O)Oc1ncnc3sc(-c4ccc(F)cc4)c-2c13] |
| 20 | 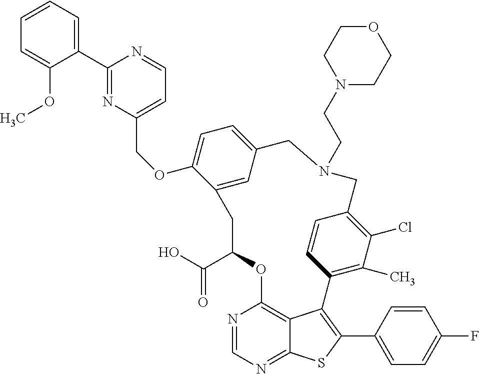 |
| 21 | 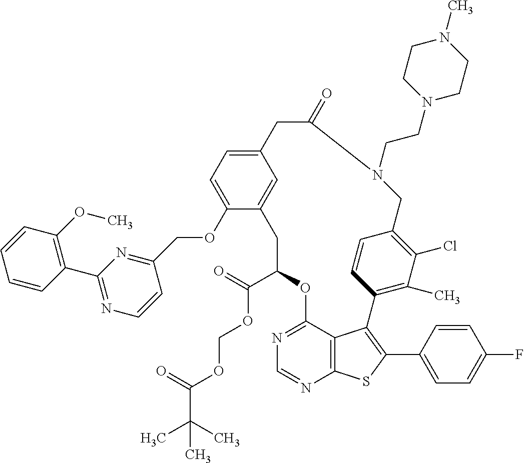 |
| 22 | 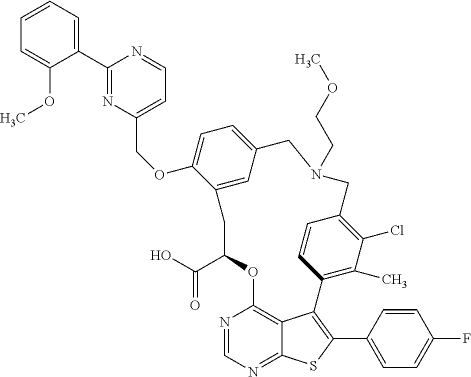 |
| 23 | 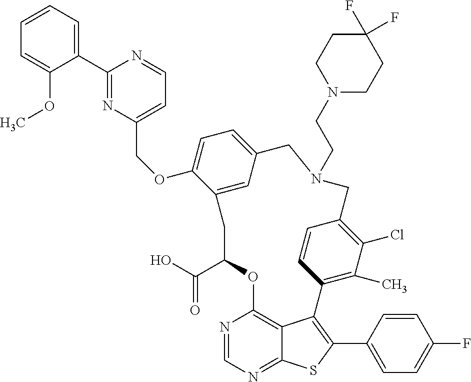 |
| 24 | 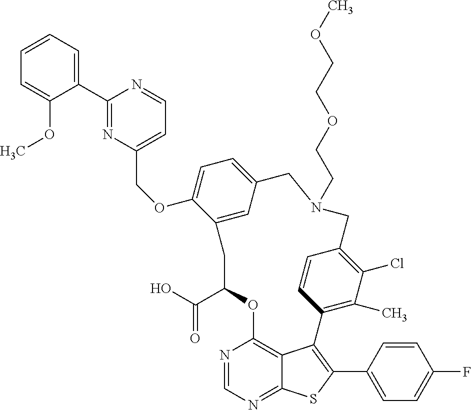 |
| 25 | 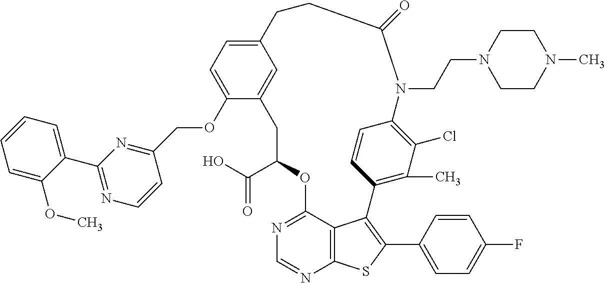 |
| 26 | 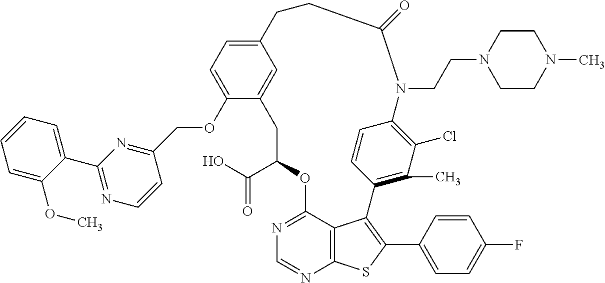 [SMILES: COc1ccccc1-c1nccc(COc2ccc3cc2C[C@H](C(=O)O)Oc2ncnc4sc(-c5ccc(F)cc5)c(c24)-c2ccc(c(Cl)c2C)N(CCN2CCN(C)CC2)C(=O)CC3)n1] |
| 27 | 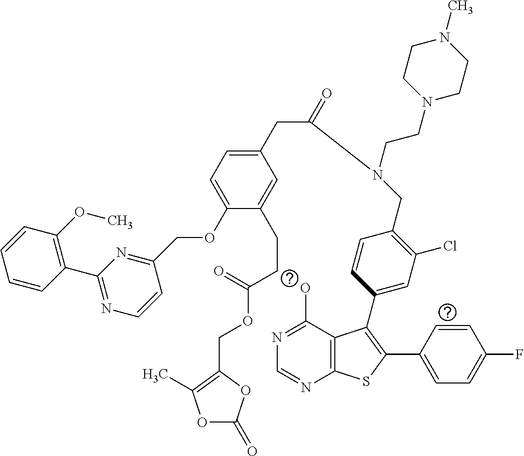 |
| 28 | 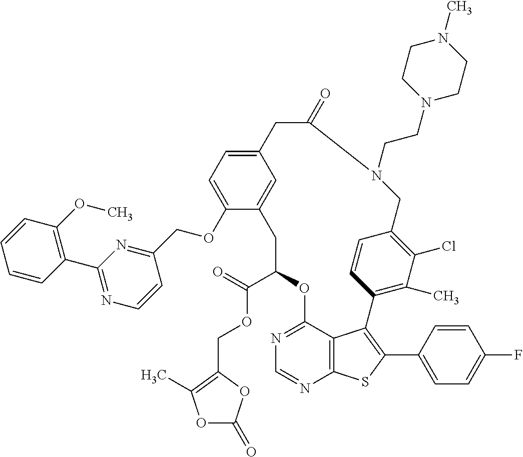 |
| 29 | 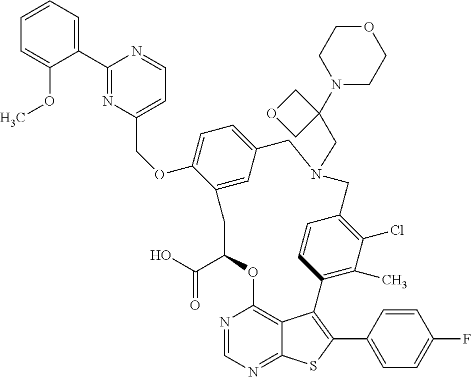 |
| 30 | 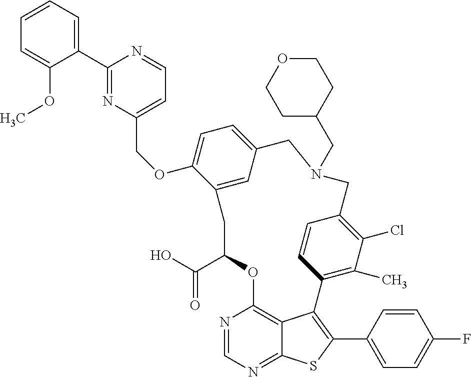 |
| 31 | 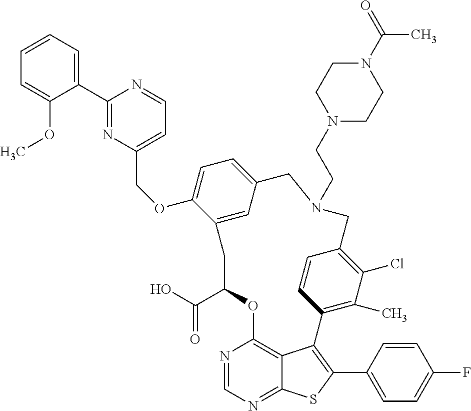 |
| 32 | 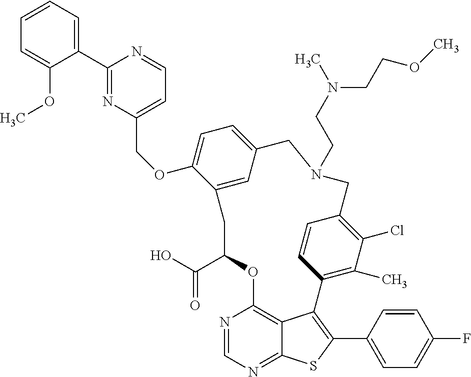 |
| 33 | 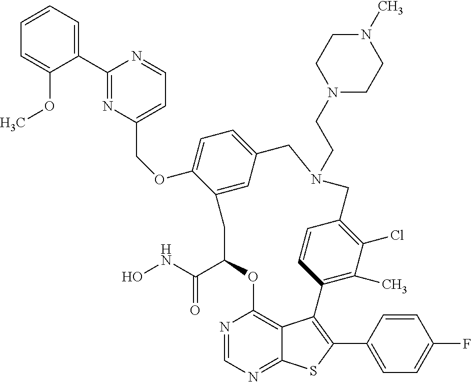 |
| 34 | 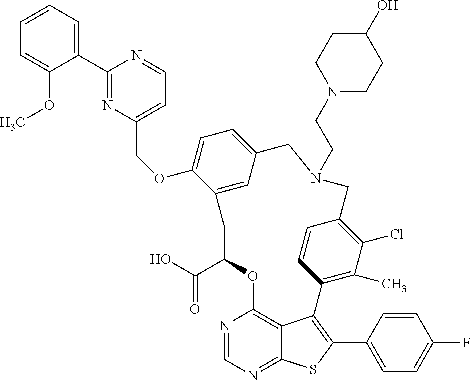 |
| 35 | 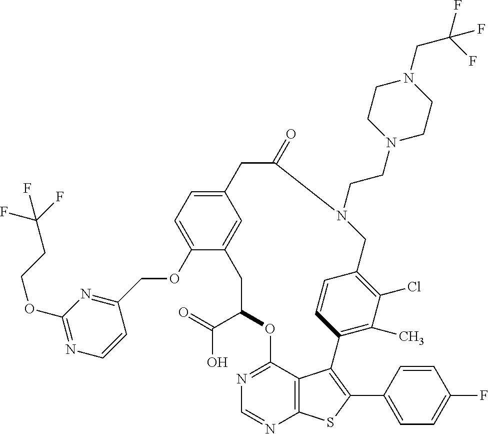 |
| 36 | 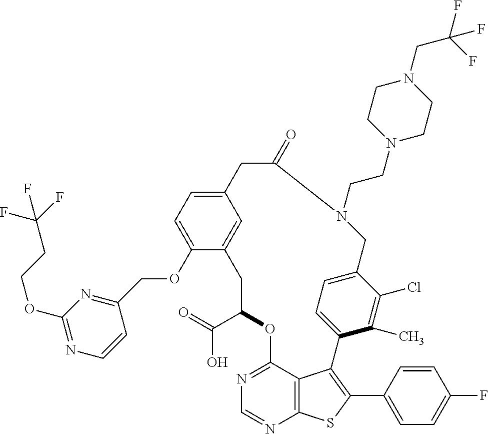 |
| 37 | 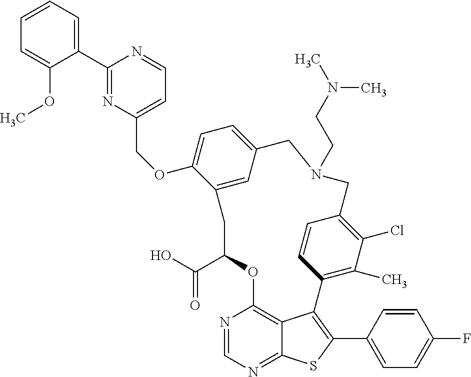 |
| 38 | 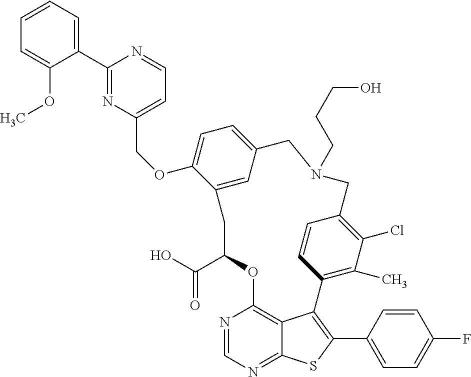 |
| 39 | 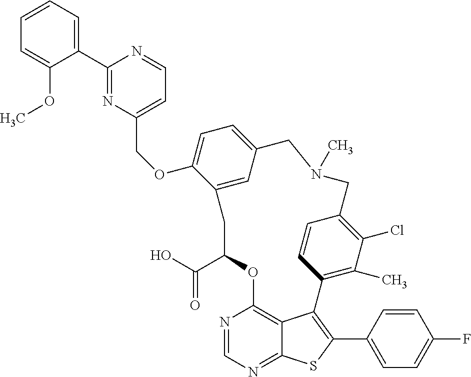 |
| 40 | 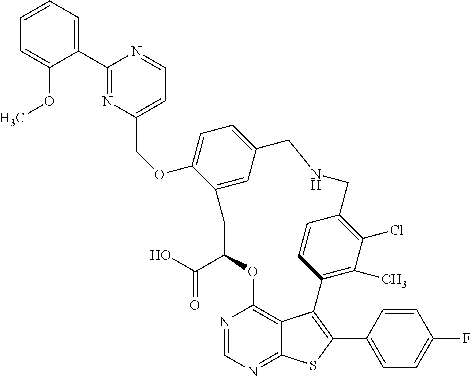 |
| 41 | 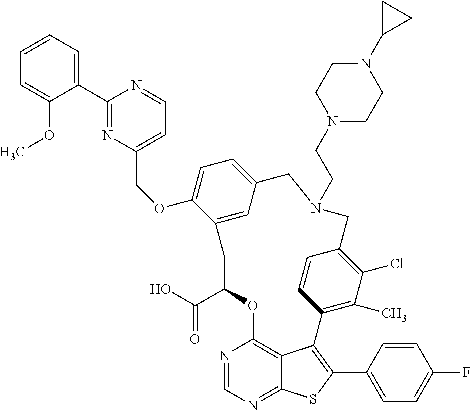 |
| 42 | 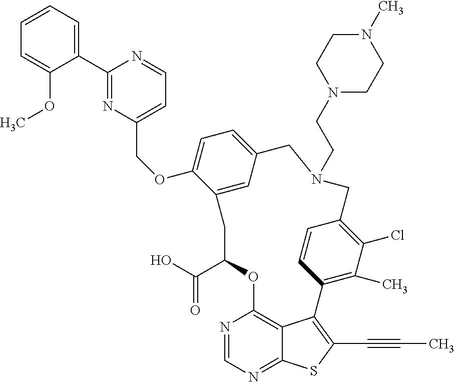 |
| 43 | 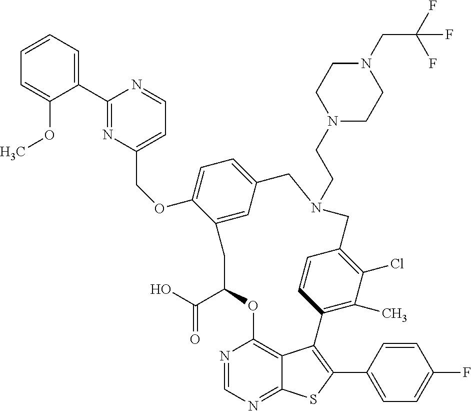 |
| 44 | 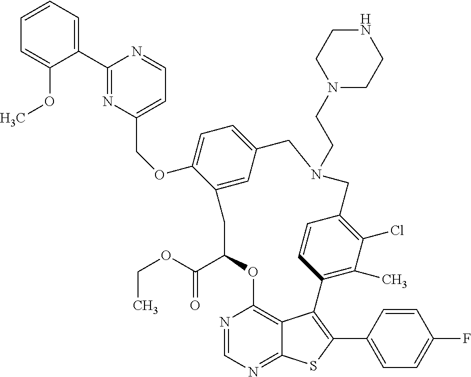 |
| 45 | 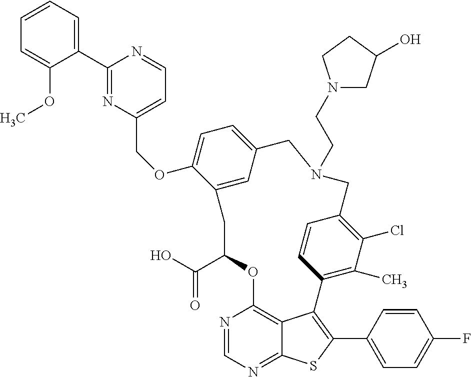 |
| 46 | 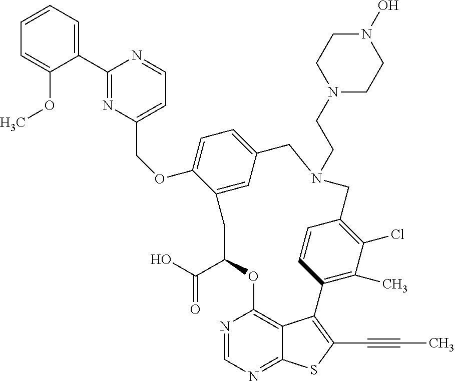 |
| 47 | 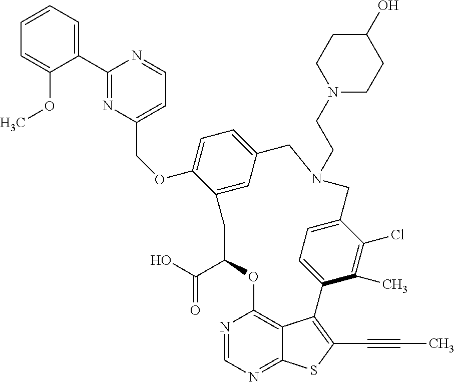 |
| 48 | 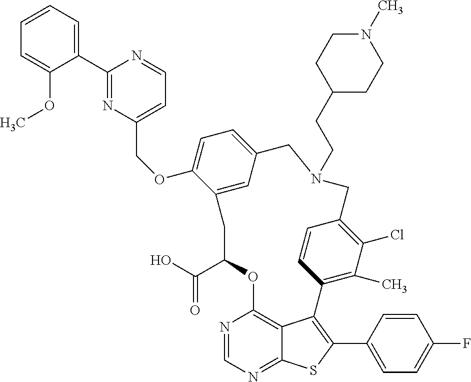 |
| 49 | 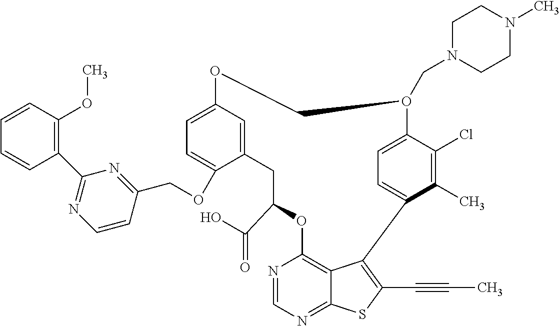 |
| 50 | 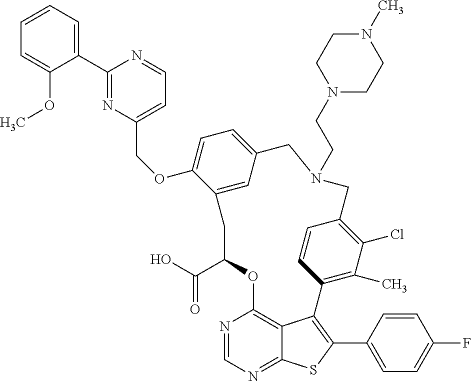 |
| 51 | 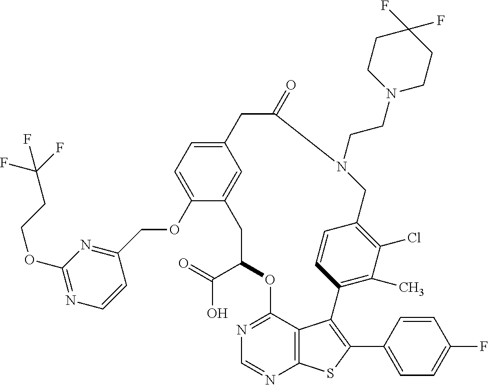 |
| 52 | 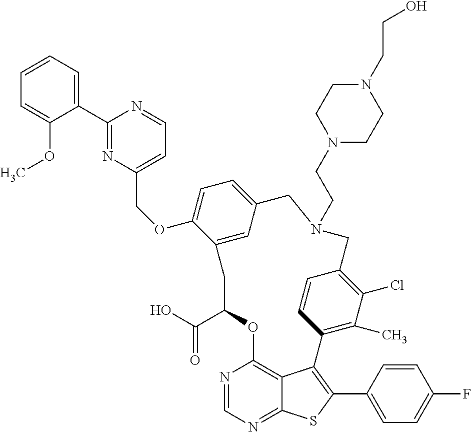 |
| 53 | 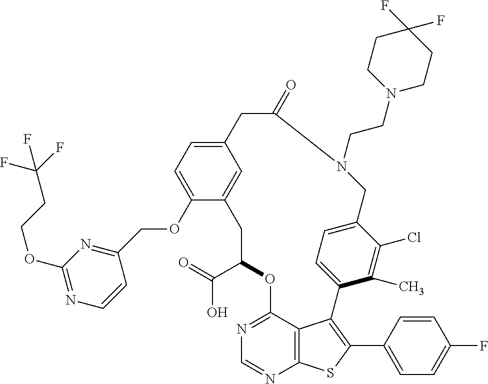 |
| 54 | 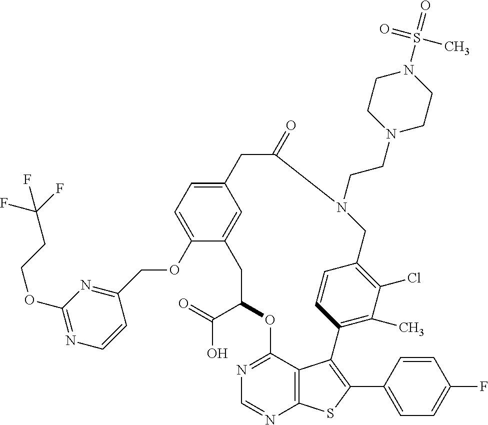 |
| 55 | 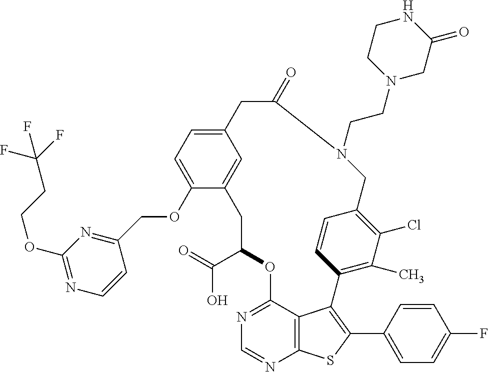 |
| 56 | 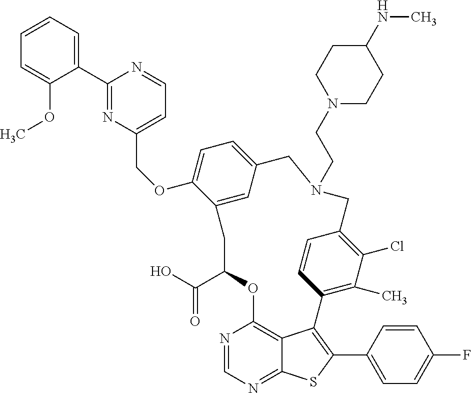 |
| 57 | 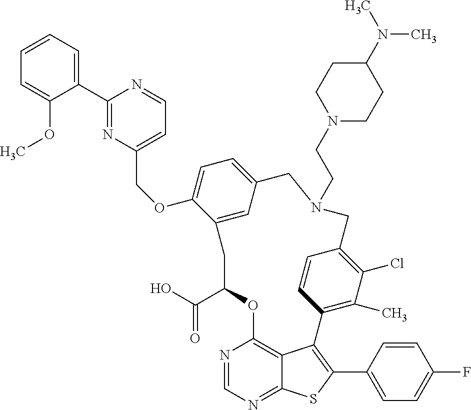 |
| 58 | 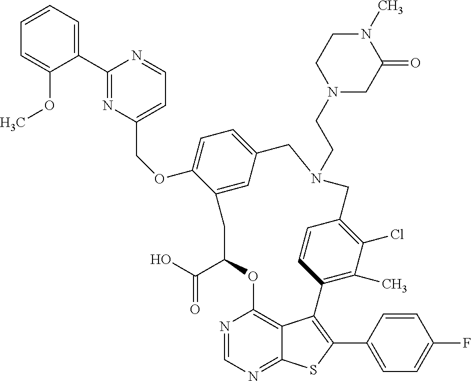 |
| 59 | 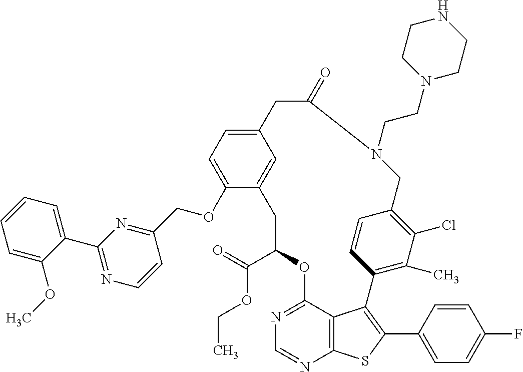 |
| 60 | 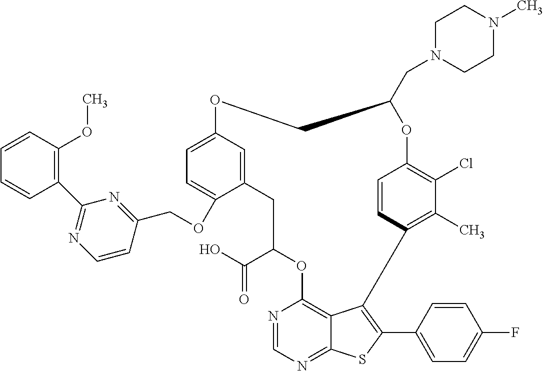 |
| 61 | 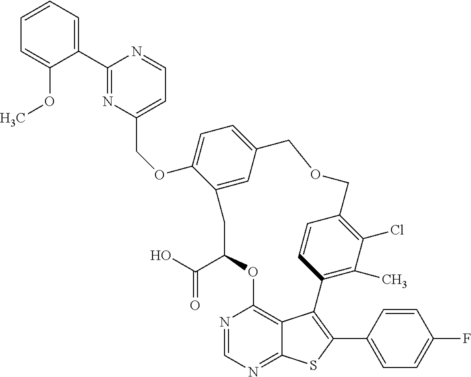 |
| 62 | 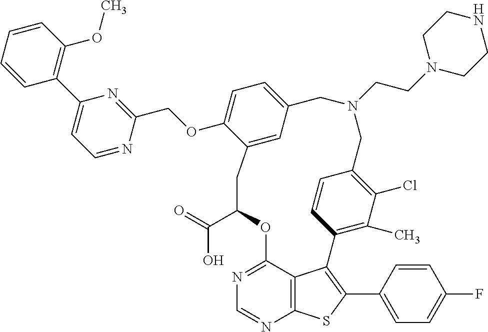 |
| 63 | 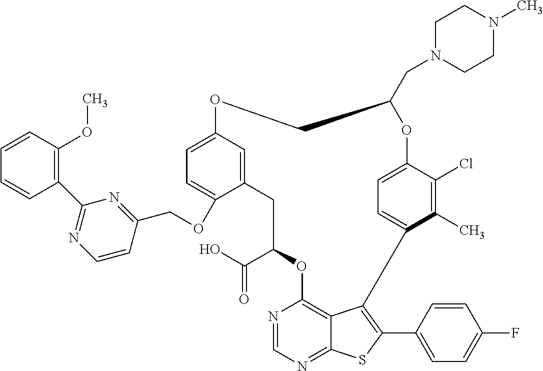 |
| 64 | 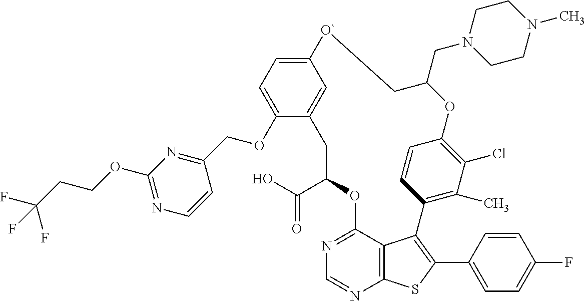 |
| 65 | 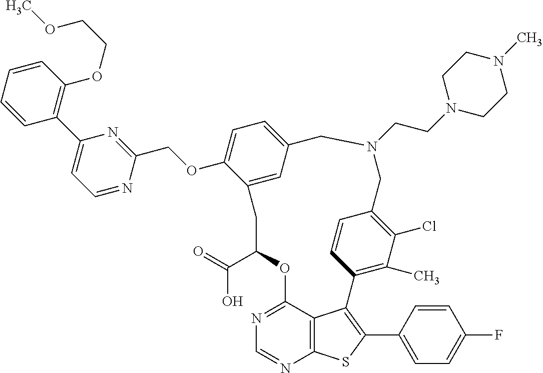 |
| 66 | 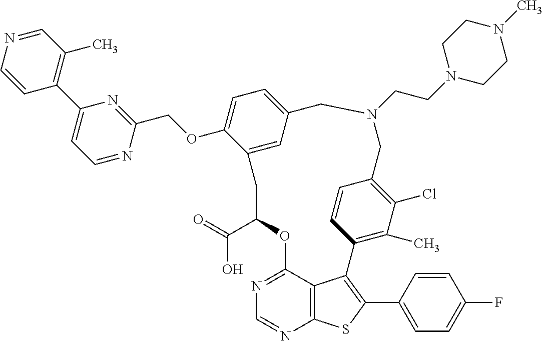 |
| 67 | 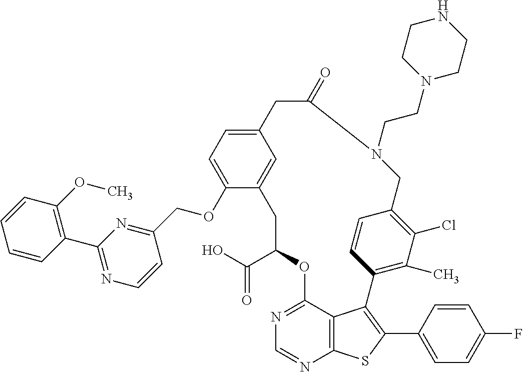 |
| 68 | 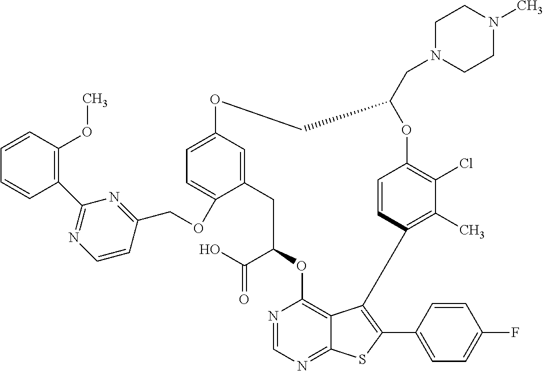 |
| 69 | 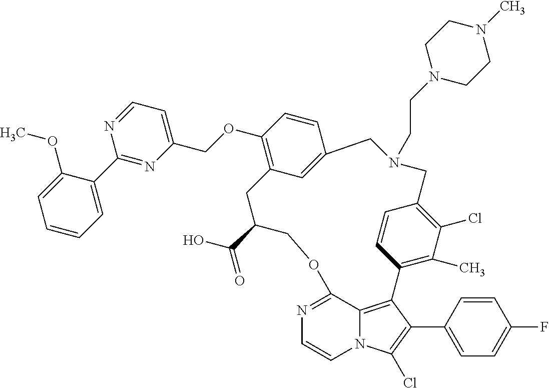 |
| 70 | 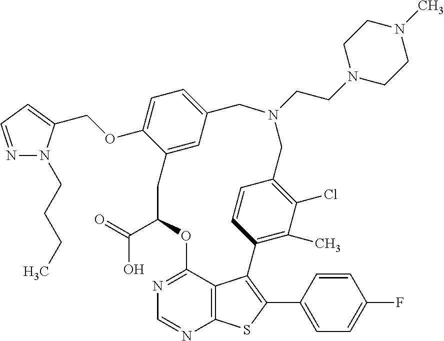 |
| 71 | 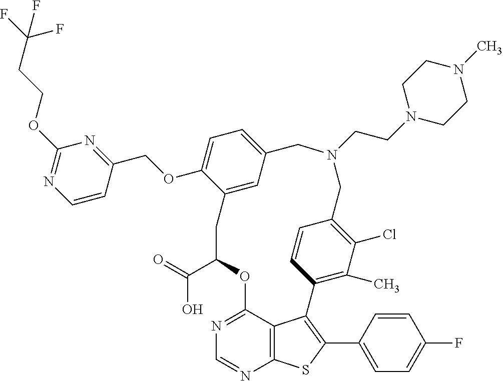 |
| 72 | 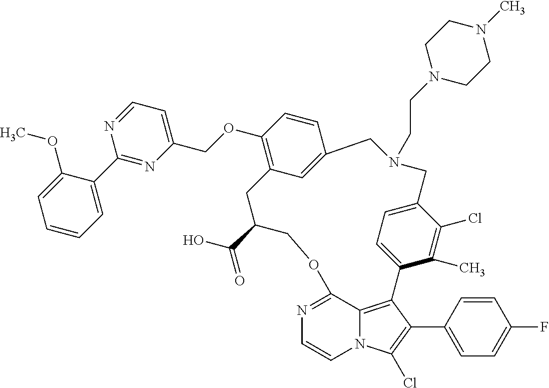 |
| 73 | 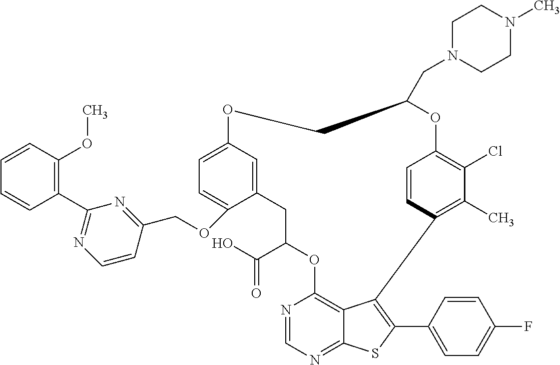 |
| 74 | 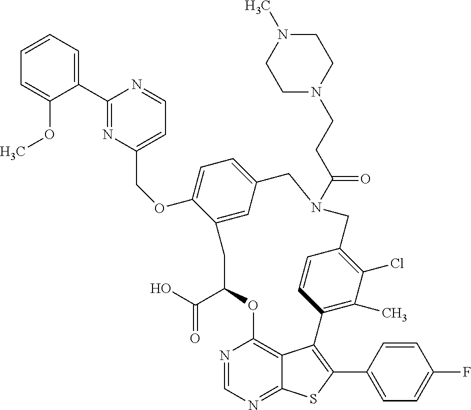 |
| 75 | 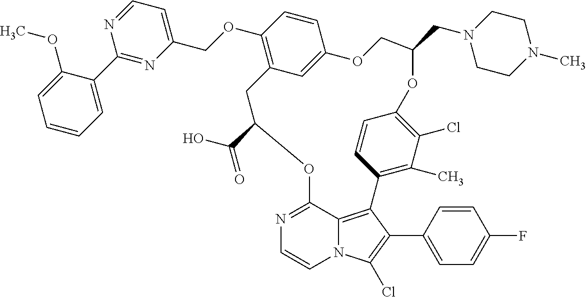 |
| 76 | 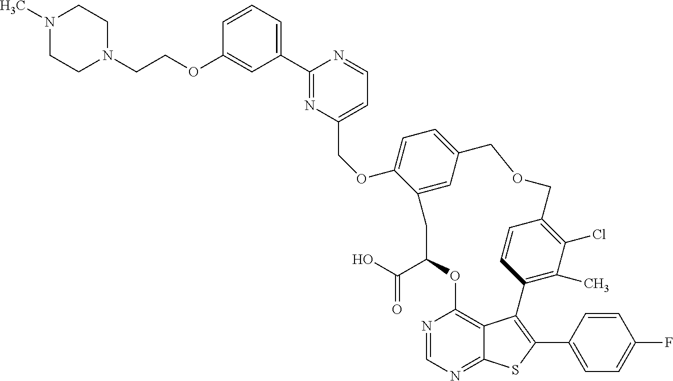 |
| 77 | 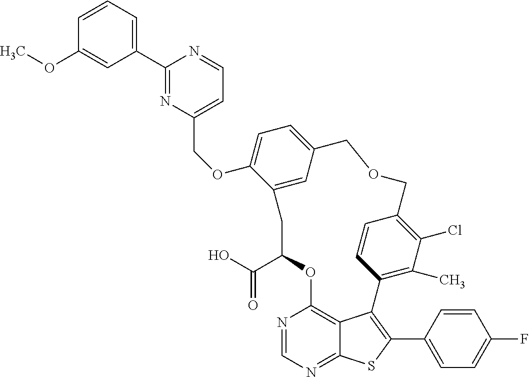 |
| 78 | 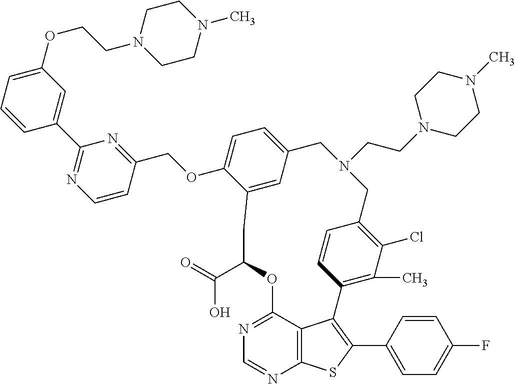 |
| 79 | 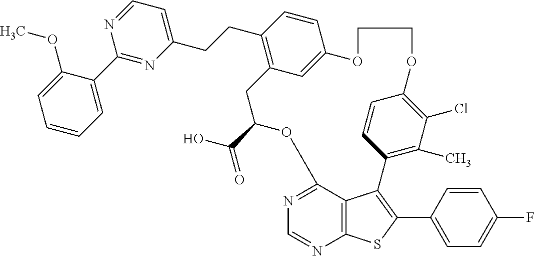 |
| 80 | 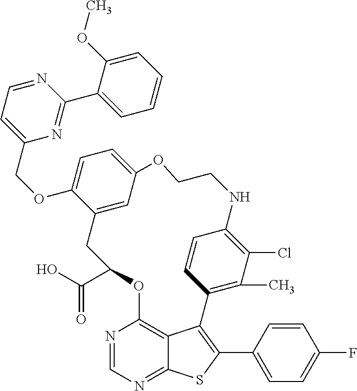 |
| 81 | 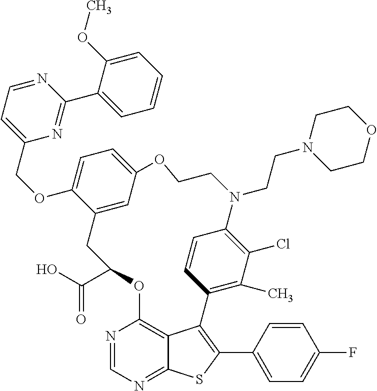 |
| 82 | 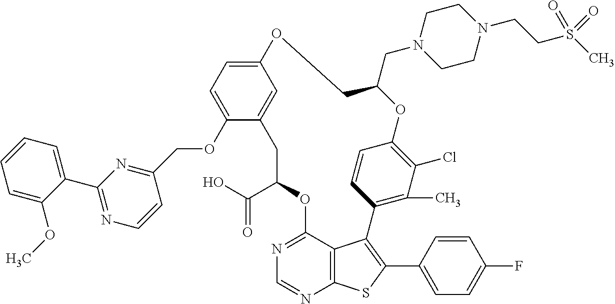 |
| 83 | 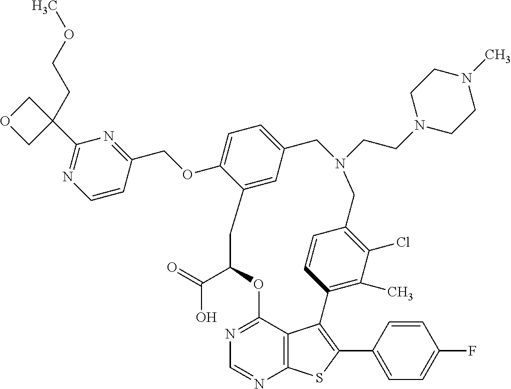 |
| 84 | 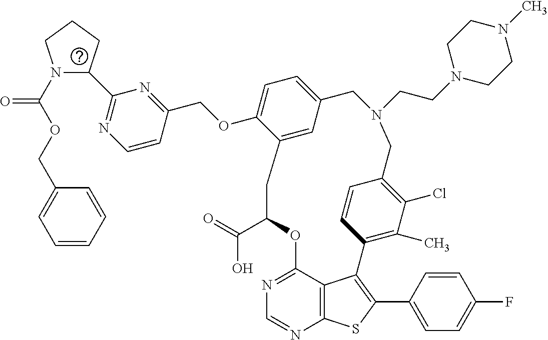 |
| 85 | 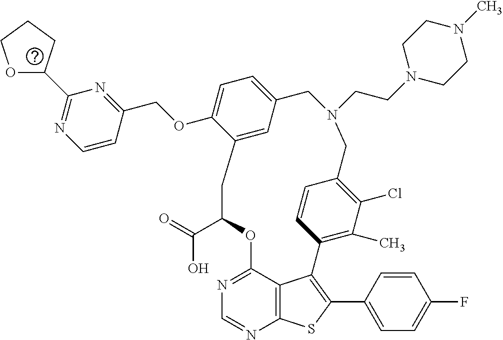 |
| 86 | 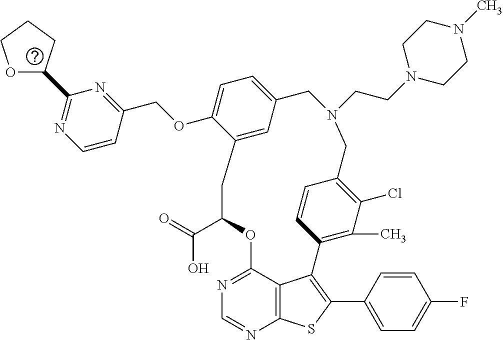 |
| 87 | 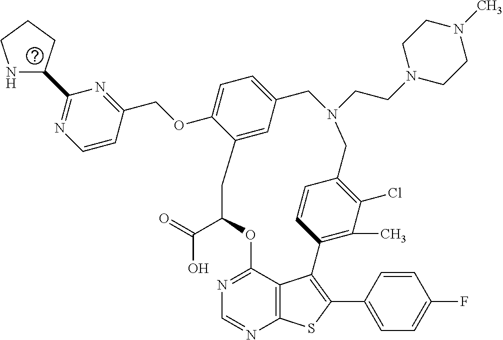 |
| 88 | 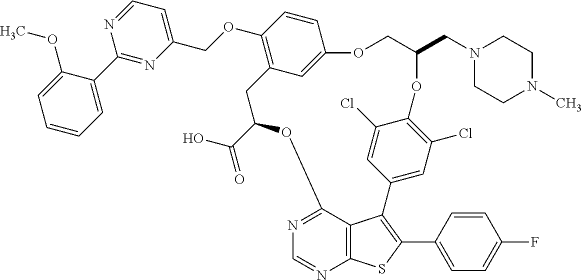 |
| 89 | 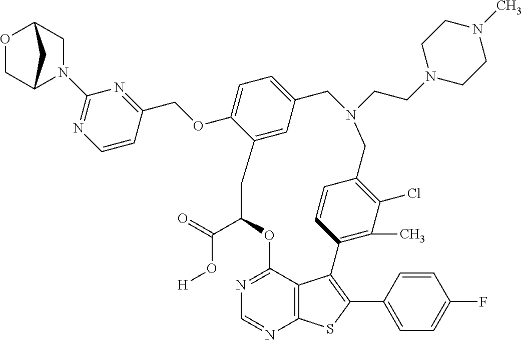 |
| 90 | 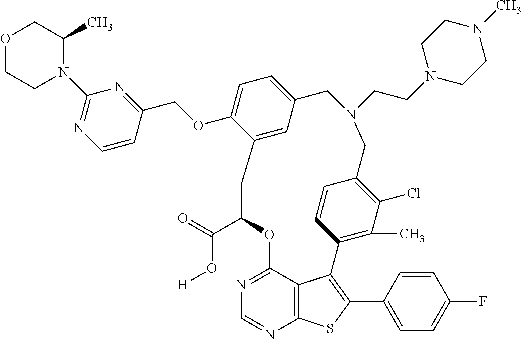 |
| 91 | 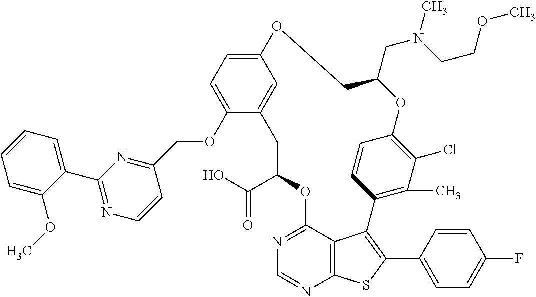 |
| 92 | 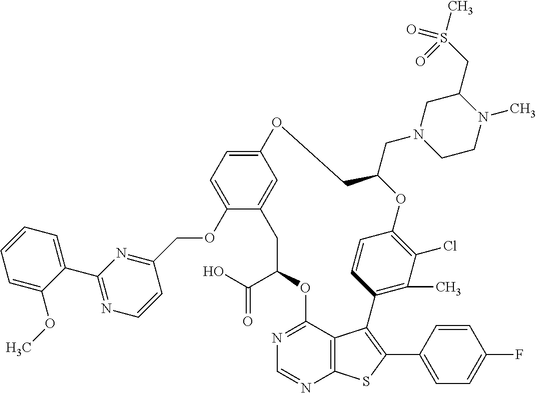 |
| 93 | 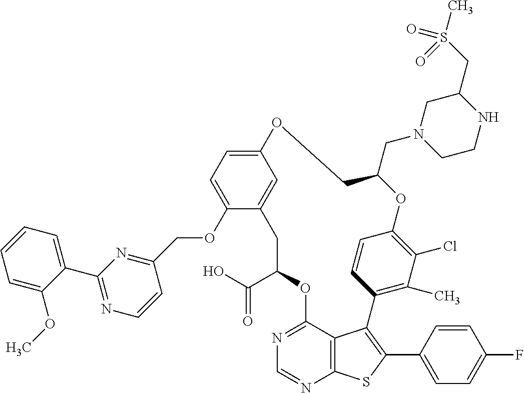 |
| 94 | 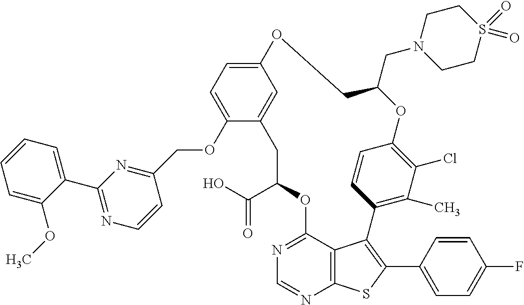 |
| 95 | 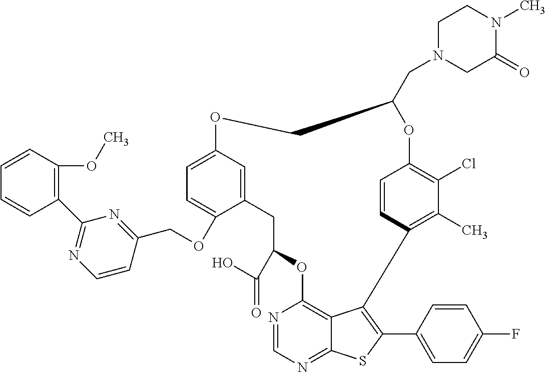 |
| 96 | 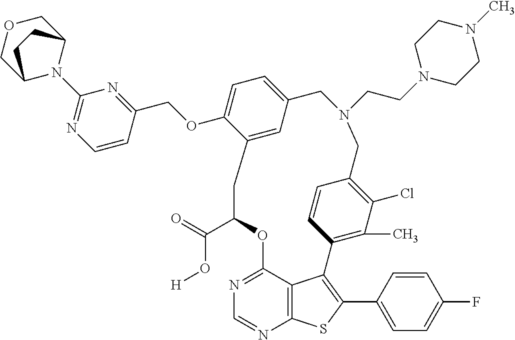 |
| 97 | 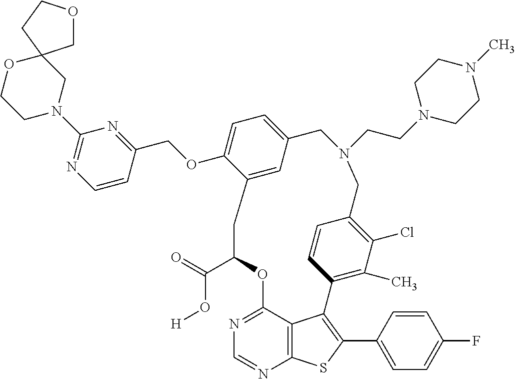 |
| 98 | 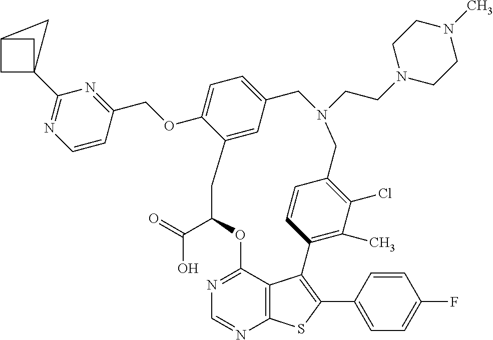 |
| 99 | 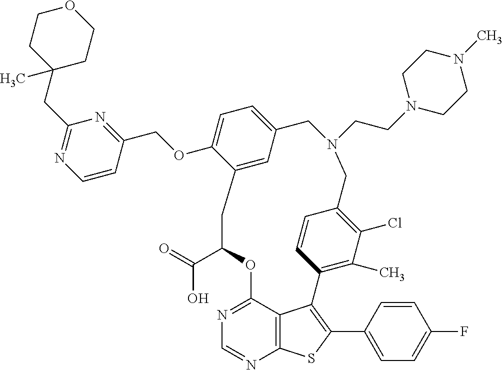 |
| 100 | 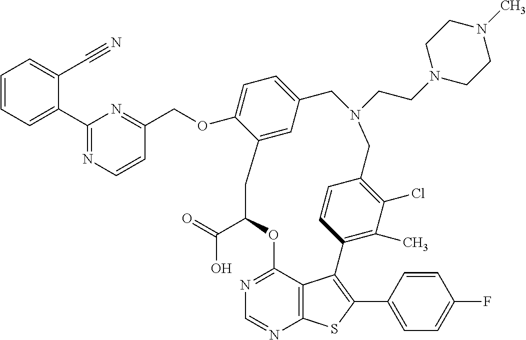 |
| 101 | 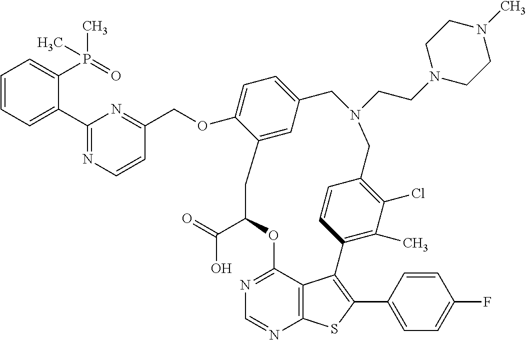 |
| 102 | 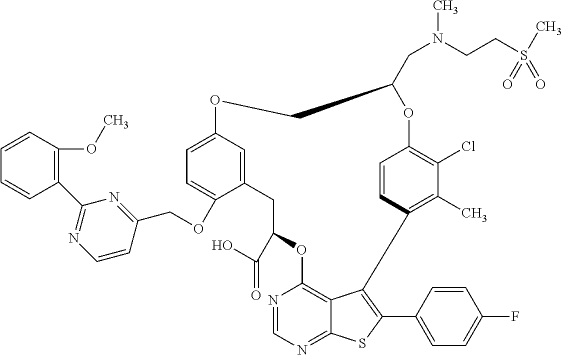 |
| 103 | 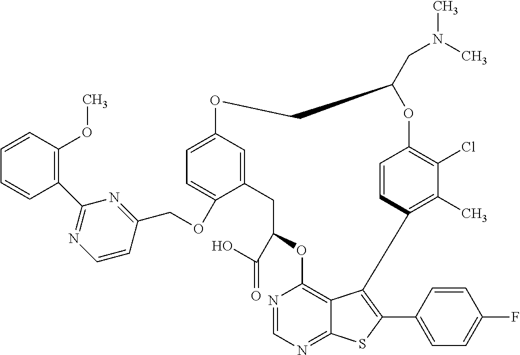 |
| 104 | 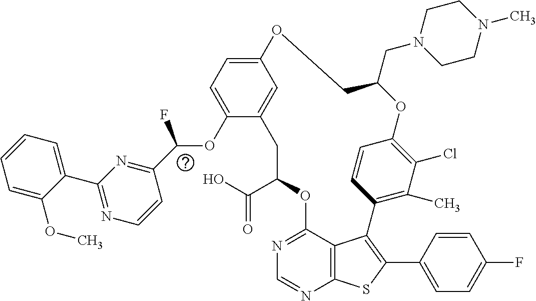 |
| 105 | 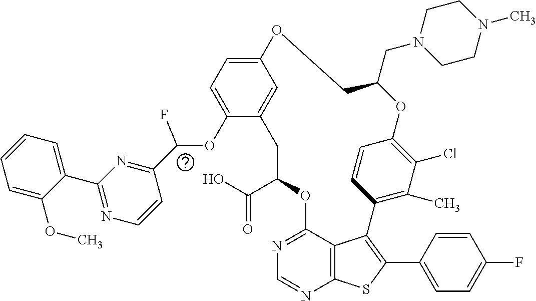 |
| 106 | 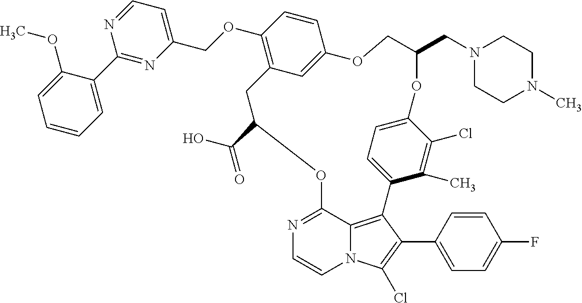 |
| 107 | 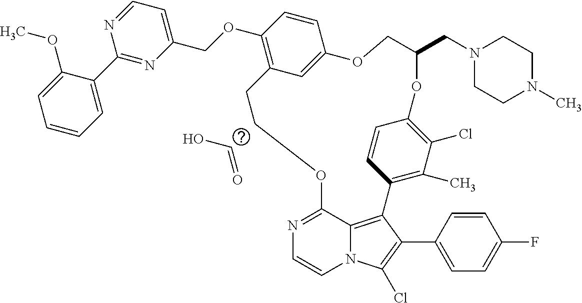 |
| 108 | 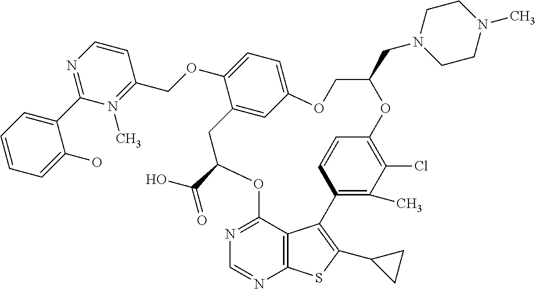 |
| 109 | 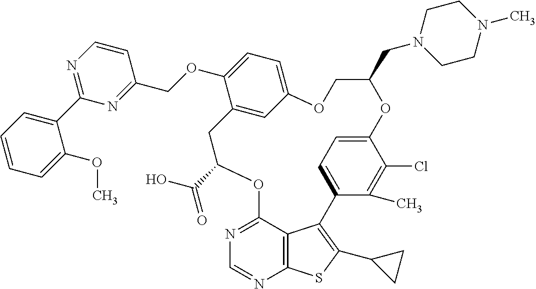 [SMILES: COc1ccccc1-c1nccc(COc2ccc3cc2C[C@@H](C(=O)O)Oc2ncnc4sc(C5CC5)c(c24)-c2ccc(c(Cl)c2C)O[C@H](CN2CCN(C)CC2)CO3)n1] |
| 110 | 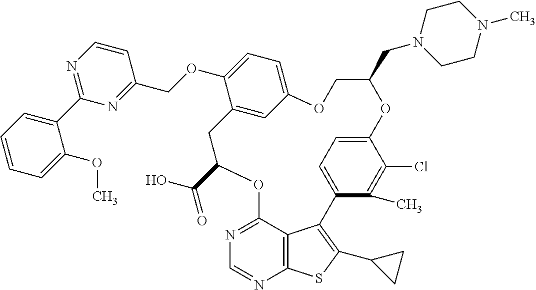 |
| 111 | 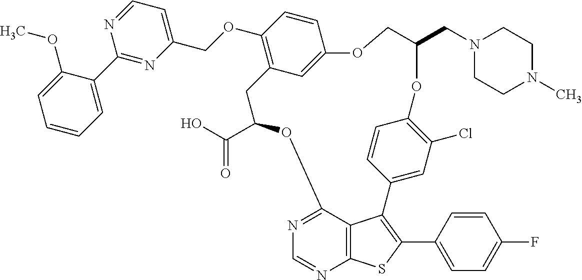 |
| 112 | 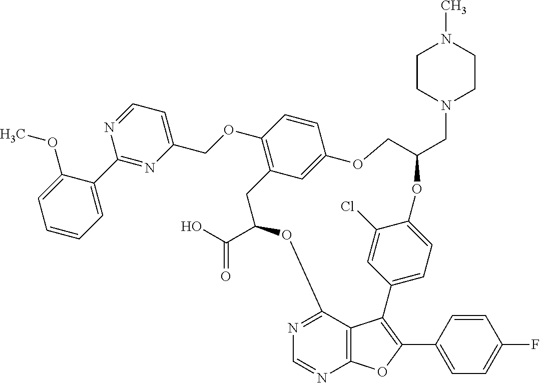 [SMILES: COc1ccccc1-c1nccc(COc2ccc3cc2C[C@H](C(=O)O)Oc2ncnc4oc(-c5ccc(F)cc5)c(c24)-c2ccc(c(Cl)c2)O[C@H](CN2CCN(C)CC2)CO3)n1] |
| 113 | 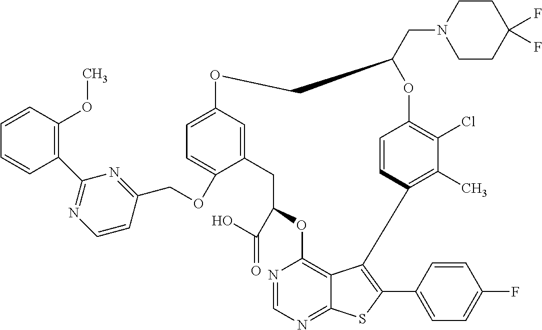 |
| 114 | 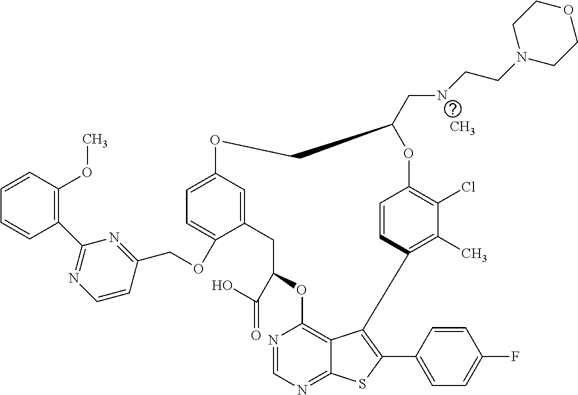 |
| 115 | 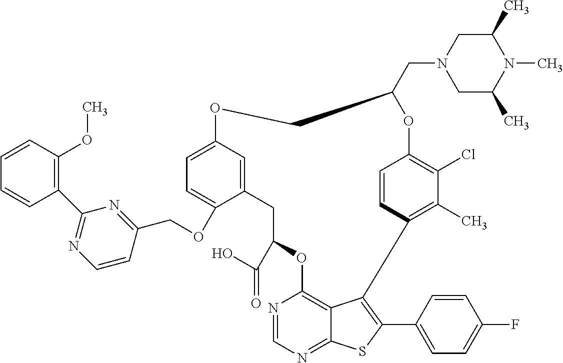 |
| 116 | 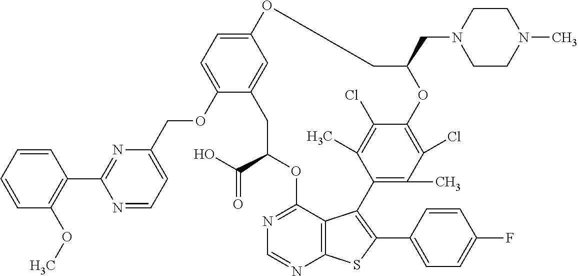 [SMILES: COc1ccccc1-c1nccc(COc2ccc3cc2C[C@H](C(=O)O)Oc2ncnc4sc(-c5ccc(F)cc5)c(c24)-c2c(C)c(Cl)c(c(Cl)c2C)O[C@H](CN2CCN(C)CC2)CO3)n1] |
| 117 | 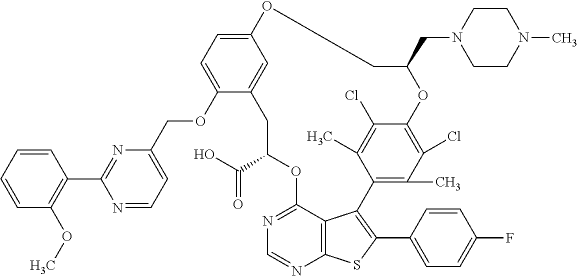 [SMILES: COc1ccccc1-c1nccc(COc2ccc3cc2C[C@@H](C(=O)O)Oc2ncnc4sc(-c5ccc(F)cc5)c(c24)-c2c(C)c(Cl)c(c(Cl)c2C)O[C@H](CN2CCN(C)CC2)CO3)n1] |
| 118 | 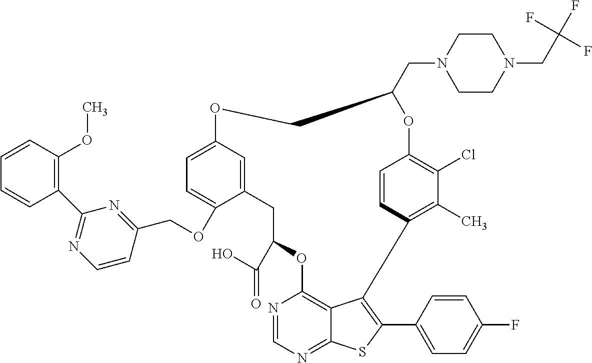 |
| 119 | 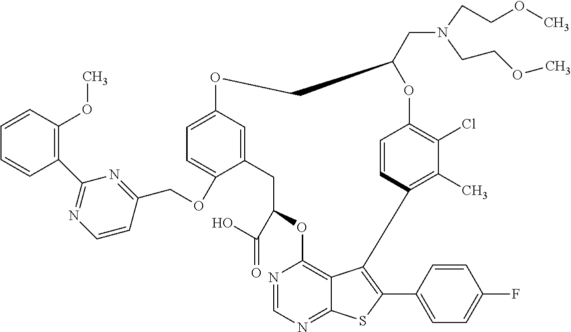 |
| 120 | 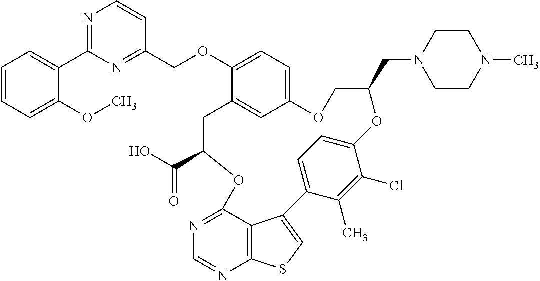 [SMILES: COc1ccccc1-c1nccc(COc2ccc3cc2C[C@H](C(=O)O)Oc2ncnc4scc(c24)-c2ccc(c(Cl)c2C)O[C@H](CN2CCN(C)CC2)CO3)n1] |
| 121 | 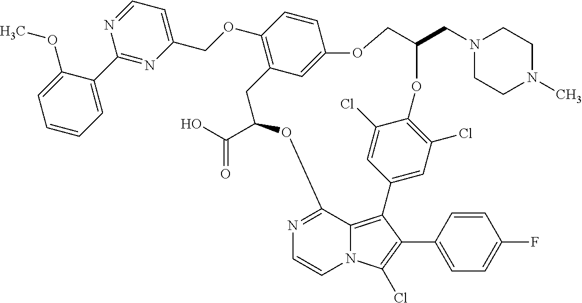 |
| 122 | 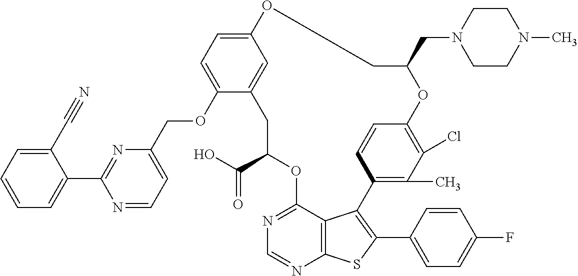 |
| 123 | 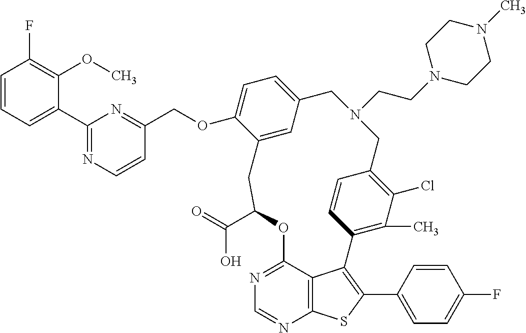 |
| 124 | 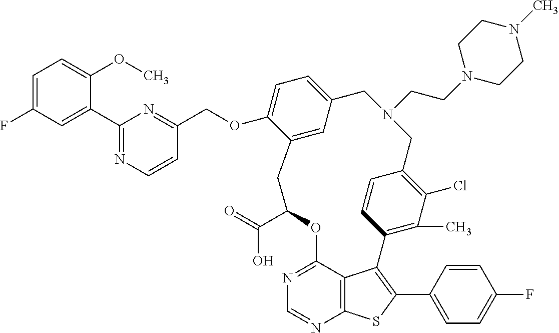 |
| 125 | 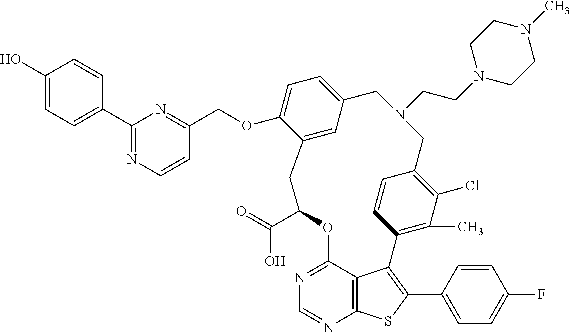 |
| 126 | 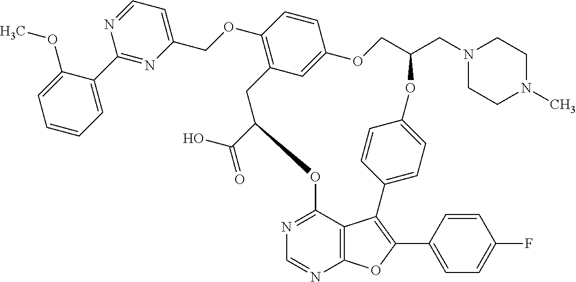 |
| 127 | 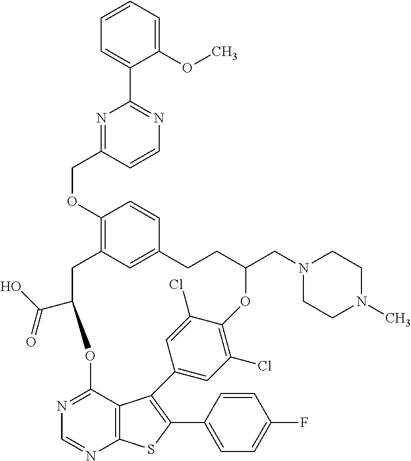 [SMILES: COc1ccccc1-c1nccc(COc2ccc3cc2C[C@H](C(=O)O)Oc2ncnc4sc(-c5ccc(F)cc5)c(c24)-c2cc(Cl)c(c(Cl)c2)OC(CN2CCN(C)CC2)CC3)n1] |
| 128 | 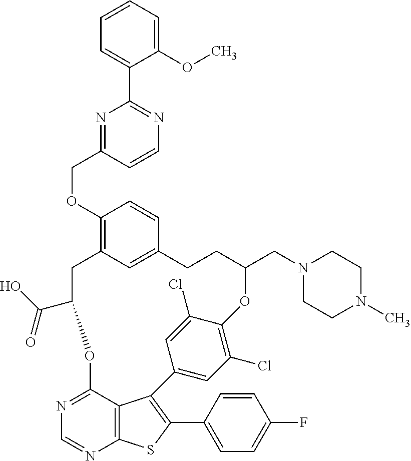 [SMILES: COc1ccccc1-c1nccc(COc2ccc3cc2C[C@@H](C(=O)O)Oc2ncnc4sc(-c5ccc(F)cc5)c(c24)-c2cc(Cl)c(c(Cl)c2)OC(CN2CCN(C)CC2)CC3)n1] |
| 129 | 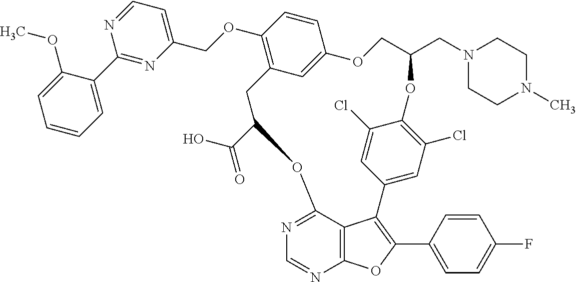 |
| 130 | 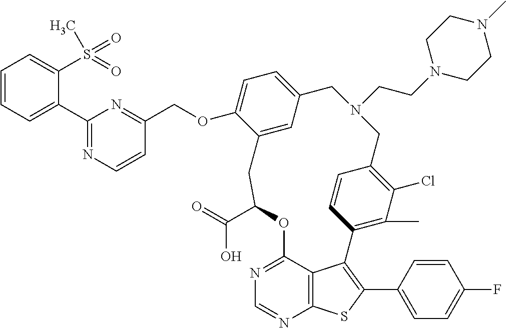 |
| 131 | 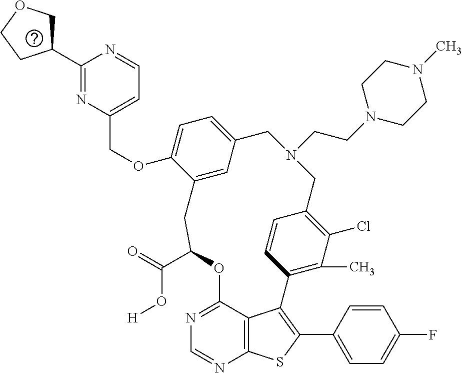 |
| 132 | 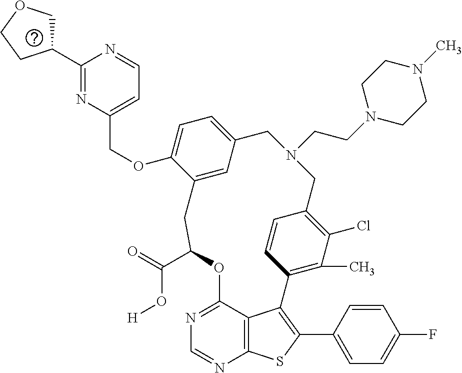 |
| 133 | 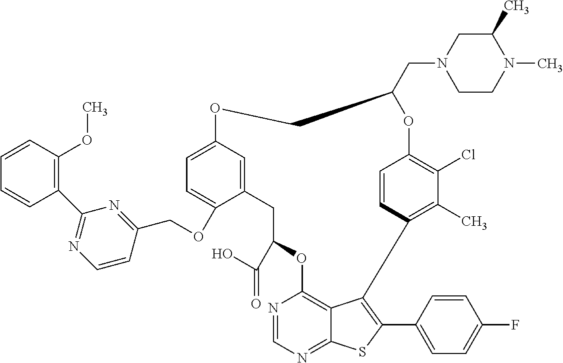 |
| 134 | 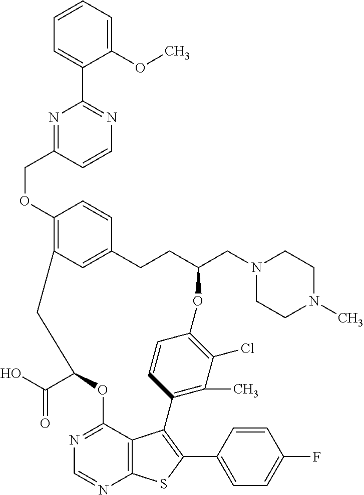 |
| 135 | 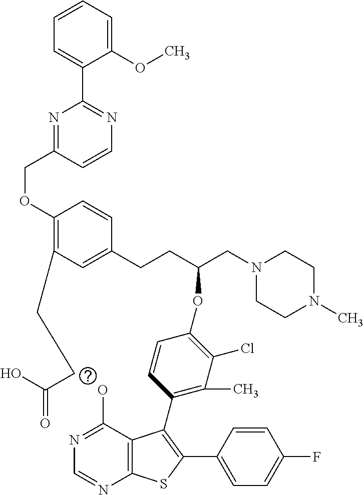 |
| 136 | 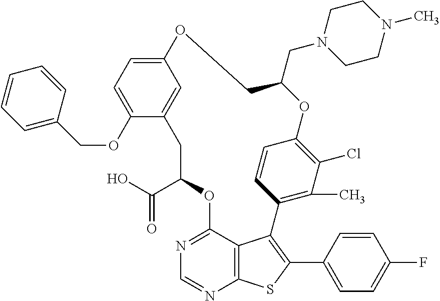 |
| 137 | 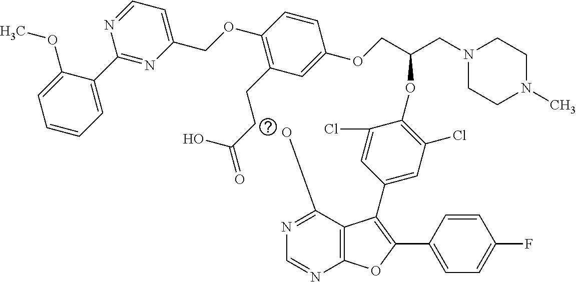 |
| 138 | 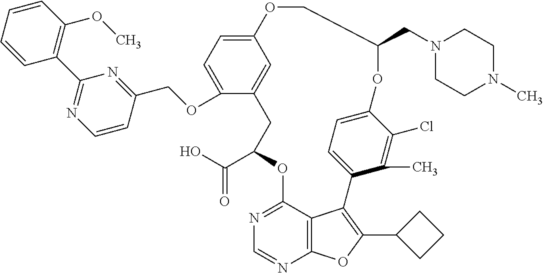 |
| 139 | 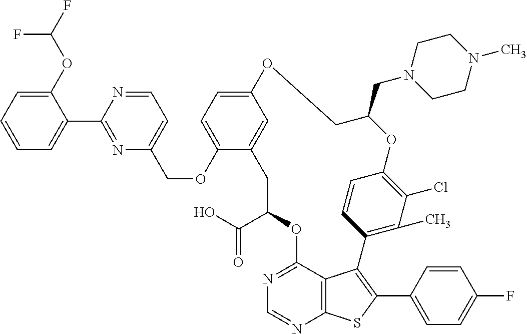 |
| 140 | 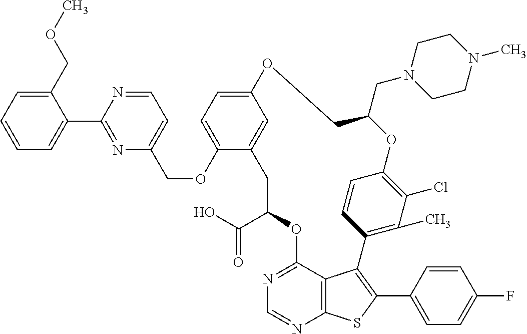 |
| 141 | 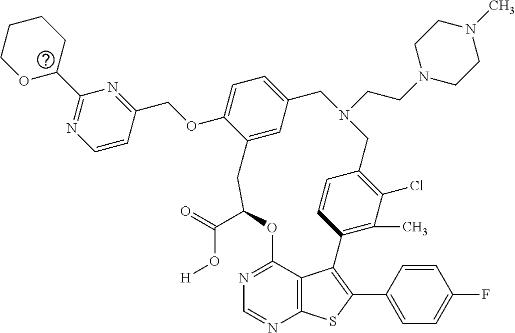 |
| 142 | 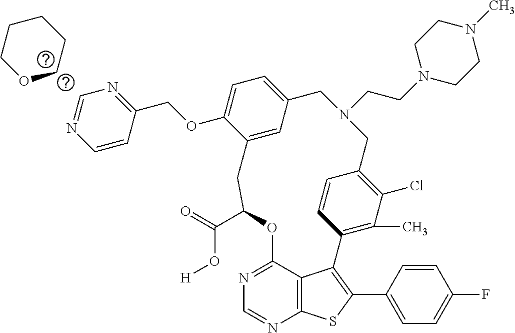 |
| 143 | 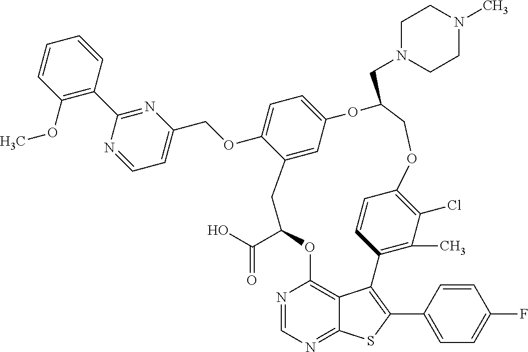 |
| 144 | 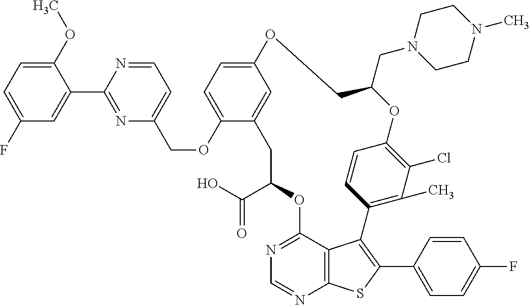 |
| 145 | 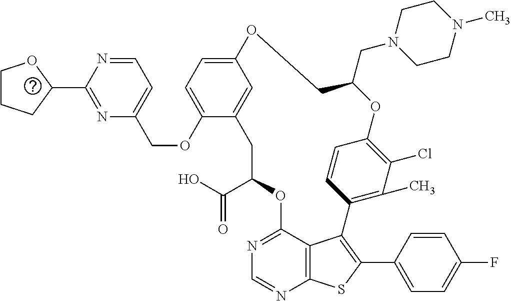 |
| 146 | 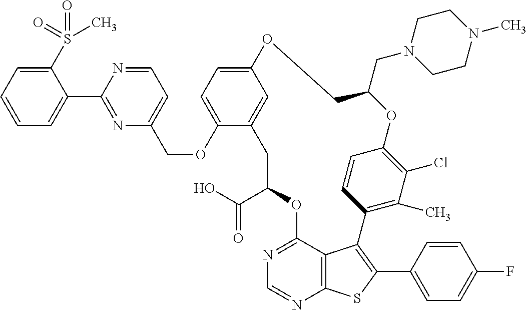 |
| 147 | 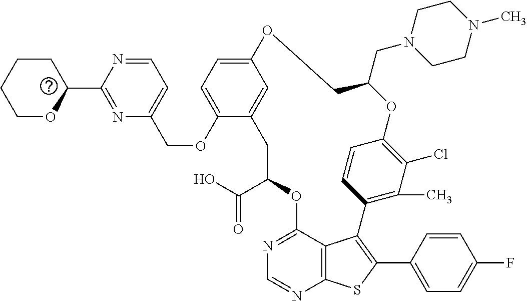 |
| 148 | 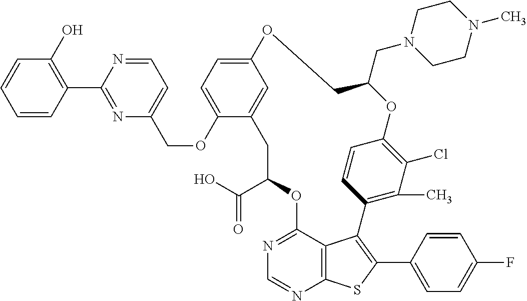 |
| 149 | 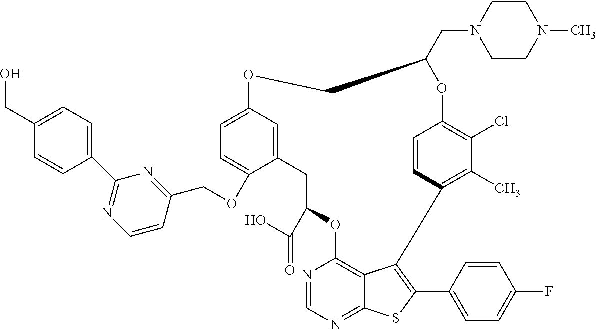 |
| 150 | 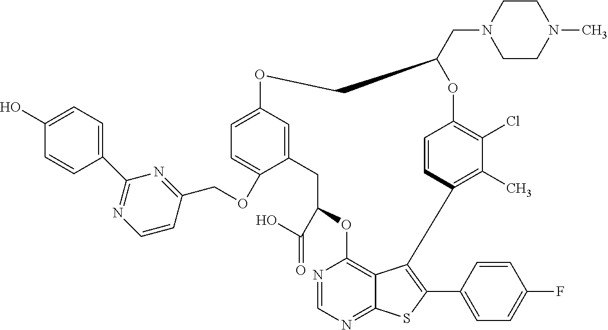 |
| 151 | 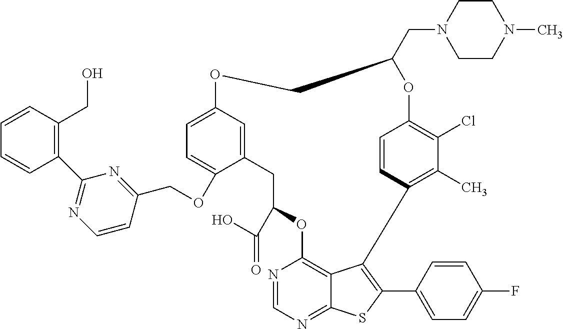 |

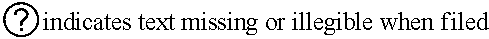

One embodiment pertains to Example 73, and pharmaceutically acceptable salts thereof:

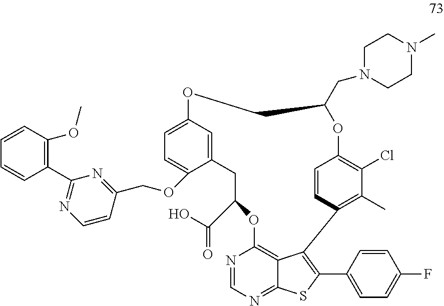

That is, in embodiments, the compound of Formula (I) is (7R,16R,21S)-19-chloro-1-(4-fluorophenyl)-10-{[2-(2-methoxyphenyl)pyrimidin-4-yl]methoxy}-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid, or pharmaceutically acceptable salts thereof.

One embodiment pertains to Example 108, and pharmaceutically acceptable salts thereof:

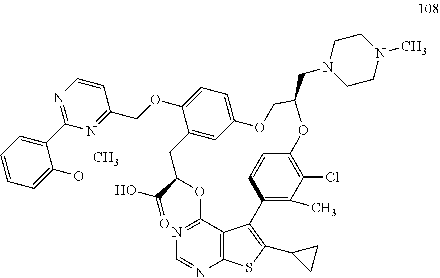

That is, in embodiments, the compound of Formula (I) is (7R,16R,21S)-19-chloro-1-cyclopropyl-10-{[2-(2-methoxyphenyl)pyrimidin-4-yl]methoxy}-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid, or pharmaceutically acceptable salts thereof.

One embodiment pertains to Example 116, and pharmaceutically acceptable salts thereof:

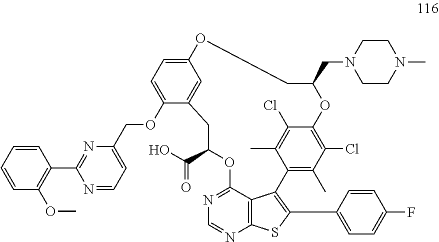 [SMILES: COc1ccccc1-c1nccc(COc2ccc3cc2C[C@H](C(=O)O)Oc2ncnc4sc(-c5ccc(F)cc5)c(c24)-c2c(C)c(Cl)c(c(Cl)c2C)O[C@H](CN2CCN(C)CC2)CO3)n1]

That is, in embodiments, the compound of Formula (I) is (7R,16R)-19,23-dichloro-1-(4-fluorophenyl)-10-{[2-(2-methoxyphenyl)pyrimidin-4-yl]methoxy}-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid, or pharmaceutically acceptable salts thereof.

One embodiment pertains to Example 130, and pharmaceutically acceptable salts thereof:

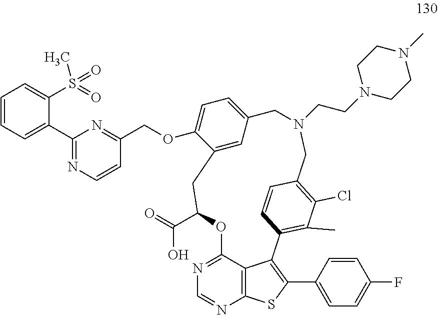

That is, in embodiments, the compound of Formula (I) is (7R,20S)-18-chloro-1-(4-fluorophenyl)-10-({2-[2-(methanesulfonyl)phenyl]pyrimidin-4-yl}methoxy)-19-methyl-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylic acid, or pharmaceutically acceptable salts thereof.

One embodiment pertains to Example 139, and pharmaceutically acceptable salts thereof:

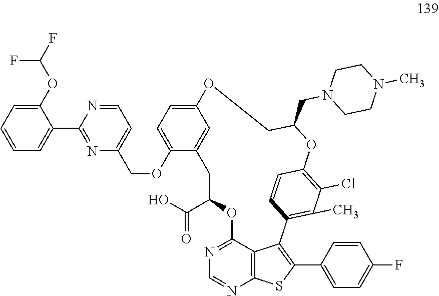

That is, in embodiments, the compound of Formula (I) is (7R,16R,21S)-19-chloro-10-({2-[2-(difluoromethoxy)phenyl]pyrimidin-4-yl}methoxy)-1-(4-fluorophenyl)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid, and pharmaceutically acceptable salts thereof.

One embodiment pertains to Example 140, and pharmaceutically acceptable salts thereof:

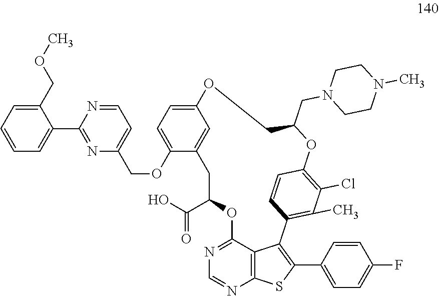

That is, in embodiments, the compound of Formula (I) is (7R,16R,21S)-19-chloro-1-(4-fluorophenyl)-10-({2-[2-(methoxymethyl)phenyl]pyrimidin-4-yl}methoxy)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid, and pharmaceutically acceptable salts thereof.

One embodiment pertains to Example 146, and pharmaceutically acceptable salts thereof:

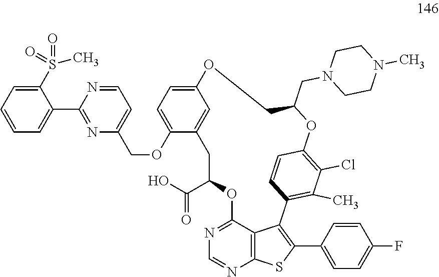

That is, in embodiments, the compound of Formula (I) is (7R,16R,21S)-19-chloro-1-(4-fluorophenyl)-10-({2-[2-(methanesulfonyl)phenyl]pyrimidin-4-yl}methoxy)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid, and pharmaceutically acceptable salts thereof.

Compounds of Formula (I) may be used in the form of pharmaceutically acceptable salts. The phrase “pharmaceutically acceptable salt” means those salts which are, within the scope of sound medical judgement, suitable for use in contact with the tissues of humans and lower animals without undue toxicity, irritation, allergic response and the like and are commensurate with a reasonable benefit/risk ratio.

Pharmaceutically acceptable salts have been described in S. M. Berge et al. J. Pharmaceutical Sciences, 1977, 66: 1-19.

Compounds of Formula (I) may contain either a basic or an acidic functionality, or both, and may be converted to a pharmaceutically acceptable salt, when desired, by using a suitable acid or base. The salts may be prepared in situ during the final isolation and purification of the compounds of the present disclosure.

Examples of acid addition salts include, but are not limited to acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, fumarate, hydrochloride, hydrobromide, hydroiodide, 2-hydroxyethansulfonate (isothionate), lactate, malate, maleate, methanesulfonate, nicotinate, 2-naphthalenesulfonate, oxalate, palmitoate, pectinate, persulfate, 3-phenylpropionate, picrate, pivalate, propionate, succinate, tartrate, thiocyanate, phosphate, glutamate, bicarbonate, p-toluenesulfonate and undecanoate. Examples of acids which may be employed to form pharmaceutically acceptable acid addition salts include such inorganic acids as hydrochloric acid, hydrobromic acid, sulfuric acid, and phosphoric acid and such organic acids as acetic acid, fumaric acid, maleic acid, 4-methylbenzenesulfonic acid, succinic acid and citric acid.

Basic addition salts may be prepared in situ during the final isolation and purification of compounds of this present disclosure by reacting a carboxylic acid-containing moiety with a suitable base such as, but not limited to, the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as, but not limited to, lithium, sodium, potassium, calcium, magnesium and aluminum salts and the like and nontoxic quaternary ammonia and amine cations including ammonium, tetramethylammonium, tetraethylammonium, methylamine, dimethylamine, trimethylamine, triethylamine, diethylamine, ethylamine and the like. Other examples of organic amines useful for the formation of base addition salts include ethylenediamine, ethanolamine, diethanolamine, piperidine, piperazine and the like.

### Synthesis

The compounds described herein, including compounds of general Formula (I) and specific examples, may be prepared, for example, through the reaction routes depicted in schemes 1-9. The variables A², A³, A⁴, A⁶, A⁷, A⁸, A¹⁵, R^{A}, R⁵, R⁹, R^{10A}, R^{10B}, R⁹, R¹², R¹³, R¹⁴, R¹⁵, R¹⁶, W, X, and Y used in the following schemes have the meanings as set forth in the Summary and Detailed Description sections unless otherwise noted.

Abbreviations that may be used in the descriptions of the schemes and the specific examples have the meanings listed in the table below.

| Abbreviation | Definition |
|---|---|
| μL | microliter |
| Boc | tert-butoxycarbonyl |
| br s | broad singlet |
| d | duplet |
| DCI | desorption chemical ionization |
| DCM | dichloromethane |
| dd | double duplet |
| DIEA | N,N-diisopropylethylamine |
| DMAP | dimethylaminopyridine |
| DMF | N,N-dimethylformamide |
| DMSO | dimethyl sulfoxide |
| eq or equiv | equivalents |
| ESI | electrospray ionization |
| Et | ethyl |
| g | gram |
| h | hours |
| HATU | 1-[bis(dimethylamino)methylene]- |
| | 1H-1,2,3-triazolo[4,5-b]pyridinium 3-oxid |
| | hexafluorophosphate |
| HOBt | 1-hydroxybenzotriazole hydrate |
| HPLC | high performance liquid chromatography |
| HPLC | high pressure liquid chromatography |
| kg | kilogram |
| LC/MS or LCMS | liquid chromatography-mass spectrometry |
| m | multiplet |
| Me | methyl |
| MeOH | methanol |
| mg | milligram |
| min | minute |
| mL | milliliter |
| mmol | millimoles |
| MPLC | medium pressure liquid chromatography |
| MS | mass spectrum |
| NMP | N-methylpyrrolidone |
| NMR | nuclear magnetic resonance |
| Ph | phenyl |
| ppm | parts per million |
| psi | pounds per square inch |
| s | singlet |
| SFC | supercritical fluid chromatography |
| tBuOH or t-BuOH | tert-butanol |
| TFA | trifluoroacetic acid |
| THF | tetrahydrofuran |
| TLC | thin layer chromatography |
| XPhos | 2-dicyclohexylphosphino-2′,4′,6′- |
| | triisopropylbiphenyl |

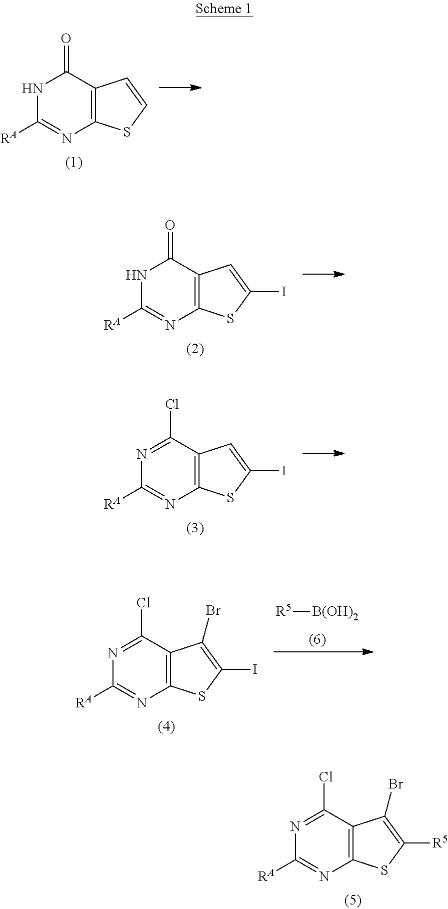

The synthesis of thienopyrimidine intermediates of Formula (5) is described in Scheme 1. Thieno[2,3-d]pyrimidine-4(3H)-ones of Formula (I), wherein R^{A} is as described herein, can be treated with periodic acid and iodine to provide 6-iodothieno[2,3-d]pyrimidin-4(3H)-ones of Formula (2). The reaction is typically performed at an elevated temperature, for example from 60° C. to 70° C., in a solvent system such as, but not limited to, acetic acid, sulfuric acid and water. 4-Chloro-6-iodothieno[2,3-d]pyrimidines of Formula (3) can be prepared by treating 6-iodothieno[2,3-d]pyrimidin-4(3H)-ones of Formula (2) with phosphorous oxychloride. The reaction is typically carried out in a solvent such as, but not limited to, N,N-dimethylaniline at an elevated temperature. 5-Bromo-4-chloro-6-iodothieno[2,3-d]pyrimidines of Formula (4) can be prepared by the treatment of 4-chloro-6-iodothieno[2,3-d]pyrimidines of Formula (3) with N-bromosuccinimide in the presence of tetrafluoroboric acid-dimethyl ether complex. The reaction is typically performed at ambient temperature in a solvent such as, but not limited to, acetonitrile. Compounds of Formula (5) can be prepared by reacting 5-bromo-4-chloro-6-iodothieno[2,3-d]pyrimidines of Formula (4) with a boronic acid (or the equivalent boronate ester) of Formula (6), wherein R⁵ is G³ as described herein, under Suzuki Coupling conditions described herein, known to those skilled in the art, or widely available in the literature.

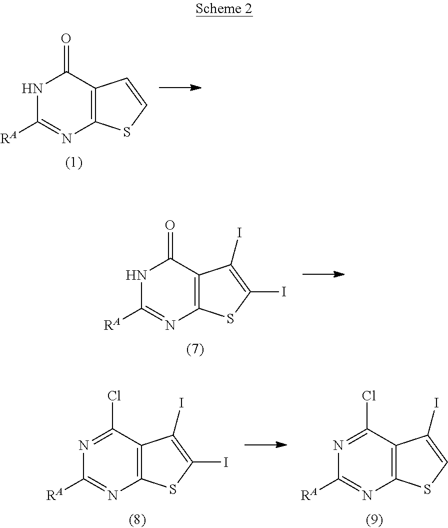

The synthesis of thienopyrimidine intermediates of Formula (9) is described in Scheme 2. Thieno[2,3-d]pyrimidine-4(3H)-ones of Formula (I), wherein R^{A} is as described herein, can be treated with periodic acid and iodine to provide 5,6-diiodothieno[2,3-d]pyrimidin-4(3H)-ones of Formula (7). The reaction is typically performed at an elevated temperature, for example from 60° C. to 100° C., in a solvent system such as, but not limited to, acetic acid, sulfuric acid and water. 4-Chloro-5,6-diiodothieno[2,3-d]pyrimidines of Formula (8) can be prepared by treating 5,6-diiodothieno[2,3-d]pyrimidin-4(3H)-ones of Formula (7) with phosphorous oxychloride. The reaction is typically carried out in a solvent such as, but not limited to, N,N-dimethylaniline at an elevated temperature. 4-Chloro-5,6-diiodothieno[2,3-d]pyrimidines of Formula (8) can be treated with tert-butylmagnesium chloride to provide compounds of Formula (9). The reaction is typically performed at a low temperature in a solvent, such as, but not limited to, tetrahydrofuran.

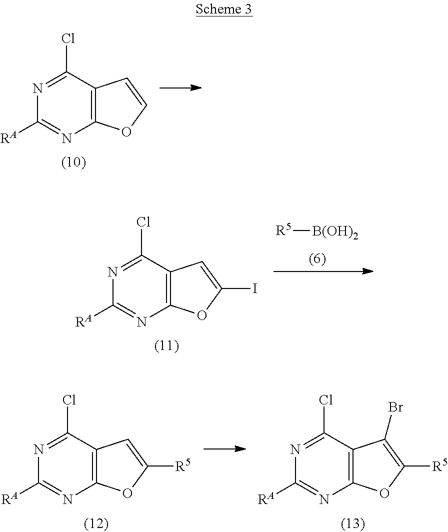

Scheme 3 describes the synthesis of furanopyrimidine intermediates of Formula (13). 4-Chlorofuro[2,3-d]pyrimidines (10), wherein R^{A} is as described herein, can be treated with lithium diisopropylamide followed by iodine, in a solvent such as, but not limited to, tetrahydrofuran, to provide 4-chloro-6-iodofuro[2,3-d]pyrimidines of Formula (11). The reaction is typically performed by first incubating a compound of Formula (10) with lithium diisopropylamide at a low temperature, such as −78° C., followed by the addition of iodine and subsequent warming to ambient temperature. Compounds of Formula (12) can be prepared by reacting 4-chloro-6-iodofuro[2,3-d]pyrimidines of Formula (11) with a boronic acid (or the equivalent boronate ester) of Formula (6) under Suzuki Coupling conditions described herein, known to those skilled in the art, or widely available in the literature. Compounds of Formula (12) can be treated with N-bromosuccinimide to provide compounds of Formula (13). The reaction is typically performed at ambient temperature in a solvent, such as, but not limited to, N,N-dimethylformamide.

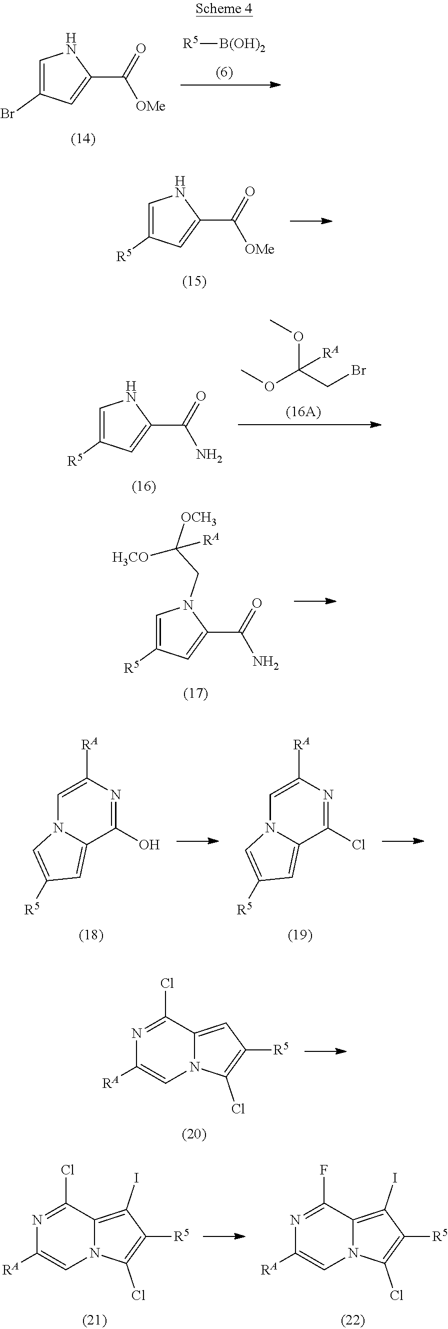

Scheme 4 describes the synthesis of pyrrolopyrazine intermediates of the Formula (22), wherein R^{A} and R⁵ are as described herein. Compounds of the Formula (15) can be prepared by reacting methyl 4-bromo-1H-pyrrole-2-carboxylate (14) with a boronic acid (or the equivalent boronate ester) of Formula (6) under Suzuki Coupling conditions described herein, known to those skilled in the art, or widely available in the literature. Compounds of Formula (15) can be heated in the presence of an aqueous ammonium hydroxide solution to provide compounds of Formula (16). Compounds of the Formula (17) can be prepared by treatment of pyrroles of Formula (16) with 2-bromo-1,1-dimethoxyethane in the presence of a base such as, but not limited to, cesium carbonate. The reaction is typically performed in a solvent such as, but not limited to, N,N-dimethylformamide at elevated temperatures ranging from 80° C. to 90° C. Compounds of Formula (17) can be treated with hydrogen chloride in a solvent such as, but not limited to, dichloromethane to provide compounds of the Formula (18). Compounds of the Formula (19) can be prepared by reacting intermediates (18) with phosphorous oxychloride in the presence of a base such as, but not limited to, N,N-diisopropylethylamine. The reaction is typically performed at elevated temperatures such as ranging from 100° C. to 115° C. Compounds of Formula (19) can be treated with N-chlorosuccinimide in a solvent system such as, but not limited to, tetrahydrofuran to provide compounds of Formula (20). The reaction is typically performed at an elevated temperature. Compounds of Formula (21) can be prepared by reacting compounds of Formula (20) with N-iodosuccinimide at an elevated temperature in a solvent such as, but not limited to, N,N-dimethylformamide. Compounds of Formula (21) can be treated with tetramethylammonium fluoride to provide compounds of Formula (22). The reaction is typically performed at ambient temperature in a solvent such as, but not limited to, N,N-dimethylformamide.

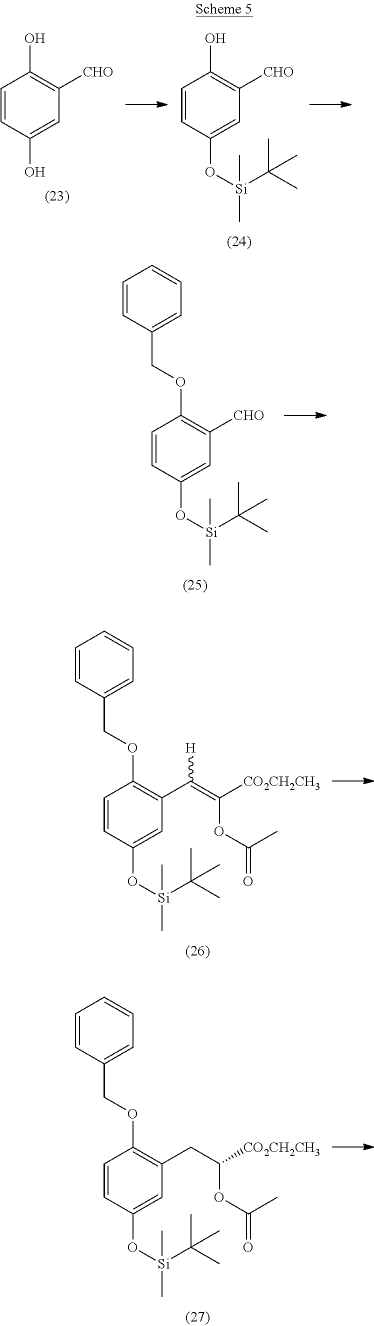

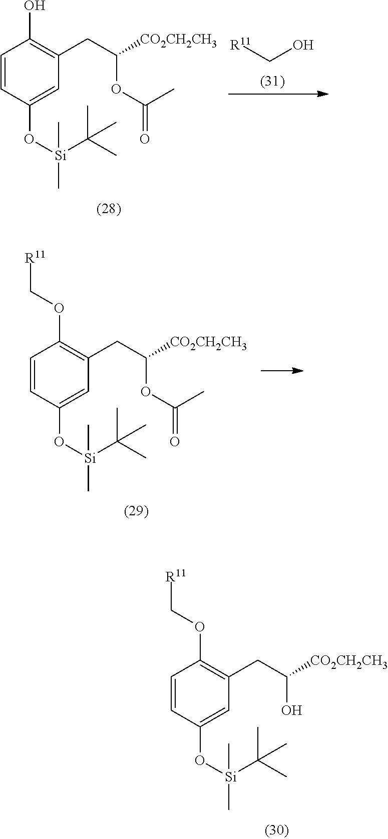

Scheme 5 describes the synthesis of propanoate intermediates of Formula (30). 2,5-Dihydroxybenzaldehyde (23) can be treated with tert-butylchlorodimethylsilane to provide mono-silylated intermediate (24). The reaction is typically conducted at ambient temperature in the presence of a base such as, but not limited to, imidazole in a solvent such as, but not limited to, dichloromethane. The mono-silylated intermediate can be reacted with benzyl bromide to provide 2-(benzyloxy)-5-((tert-butyldimethylsilyl)oxy)benzaldehyde (25). The reaction is typically performed in the presence of a base such as, but not limited to, potassium carbonate, and in a solvent such as, but not limited to acetone, N,N-dimethylformamide, or mixtures thereof. The reaction is typically initiated at room temperature followed by heating to an elevated temperature. 2-(Benzyloxy)-5-((tert-butyldimethylsilyl)oxy)benzaldehyde (25) can be treated with ethyl 2-acetoxy-2-(diethoxyphosphoryl)acetate to provide (E)/(Z)-ethyl 2-acetoxy-3-(2-(benzyloxy)-5-((tert-butyldimethylsilyl)oxy)phenyl)acrylates (26). The reaction is typically run in the presence a base such as, but not limited to, cesium carbonate in a solvent such as, but not limited to, tetrahydrofuran, toluene, or mixtures thereof. (E)/(Z)-Ethyl 2-acetoxy-3-(2-(benzyloxy)-5-((tert-butyldimethylsilyl)oxy)phenyl)acrylates (26) can be reacted with the catalyst (R,R)-Rh EtDuPhos (1,2-bis[(2R,5R)-2,5-diethylphospholano]benzene(1,5-cyclooctadiene)rhodium(I) trifluoromethanesulfonate) under an atmosphere of hydrogen gas in a solvent such as, but not limited to, methanol, to provide (R)-ethyl 2-acetoxy-3-(2-(benzyloxy)-5-((tert-butyldimethylsilyl)oxy)phenyl)propanoate (27). The reaction is typically performed at 35° C. under 50 psi of hydrogen gas. Ethyl (R)-2-acetoxy-3-(5-((tert-butyldimethylsilyl)oxy)-2-hydroxyphenyl)propanoate (28) can be provided by reacting (R)-ethyl 2-acetoxy-3-(2-(benzyloxy)-5-((tert-butyldimethylsilyl)oxy)phenyl)propanoate (27) under hydrogenolysis conditions, such as in the presence of 5% palladium on carbon under 50 psi of hydrogen gas in a solvent such as, but not limited to, ethanol at an elevated temperature, such as, but not limited to, 35° C. Ethyl (R)-2-acetoxy-3-(5-((tert-butyldimethylsilyl)oxy)-2-hydroxyphenyl)propanoate (28) can be reacted with compounds of Formula (31), wherein R¹¹ is as described herein, under Mitsunobu conditions described herein, known to those skilled in the art, or widely available in the literature, to provide compounds of Formula (29). Compounds of the Formula (29) can be treated with ethanol in the presence of a base such as, but not limited to, potassium carbonate or sodium ethoxide, to provide compounds of the Formula (30).

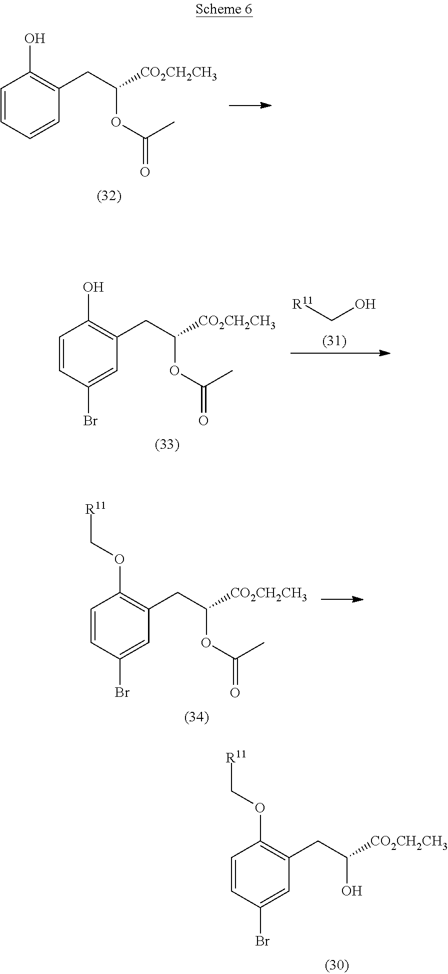

Scheme 6 describes the synthesis of propanoate intermediates of Formula (35). (R)-Ethyl 2-acetoxy-3-(2-hydroxyphenyl)propanoate (32), which can be prepared using methods similar to those described for compounds of Formula (28) in Scheme 5 or using methods described herein, can be treated with a brominating agent such as N-bromosuccinimide to provide (R)-ethyl 2-acetoxy-3-(5-bromo-2-hydroxyphenyl)propanoate (33). The reaction is typically performed in a solvent such as, but not limited to, tetrahydrofuran, at a low temperature, such as −30° C. to 0° C., before warming to ambient temperature. (R)-Ethyl 2-acetoxy-3-(5-bromo-2-hydroxyphenyl)propanoate (33) can be reacted with compounds of Formula (31), wherein R¹¹ is as described herein, under Mitsunobu conditions described herein or in the literature to provide compounds of Formula (34). Compounds of Formula (34) can be treated with ethanol in the presence of a base such as, but not limited to, potassium carbonate or sodium ethoxide at ambient temperature to provide compounds of Formula (35).

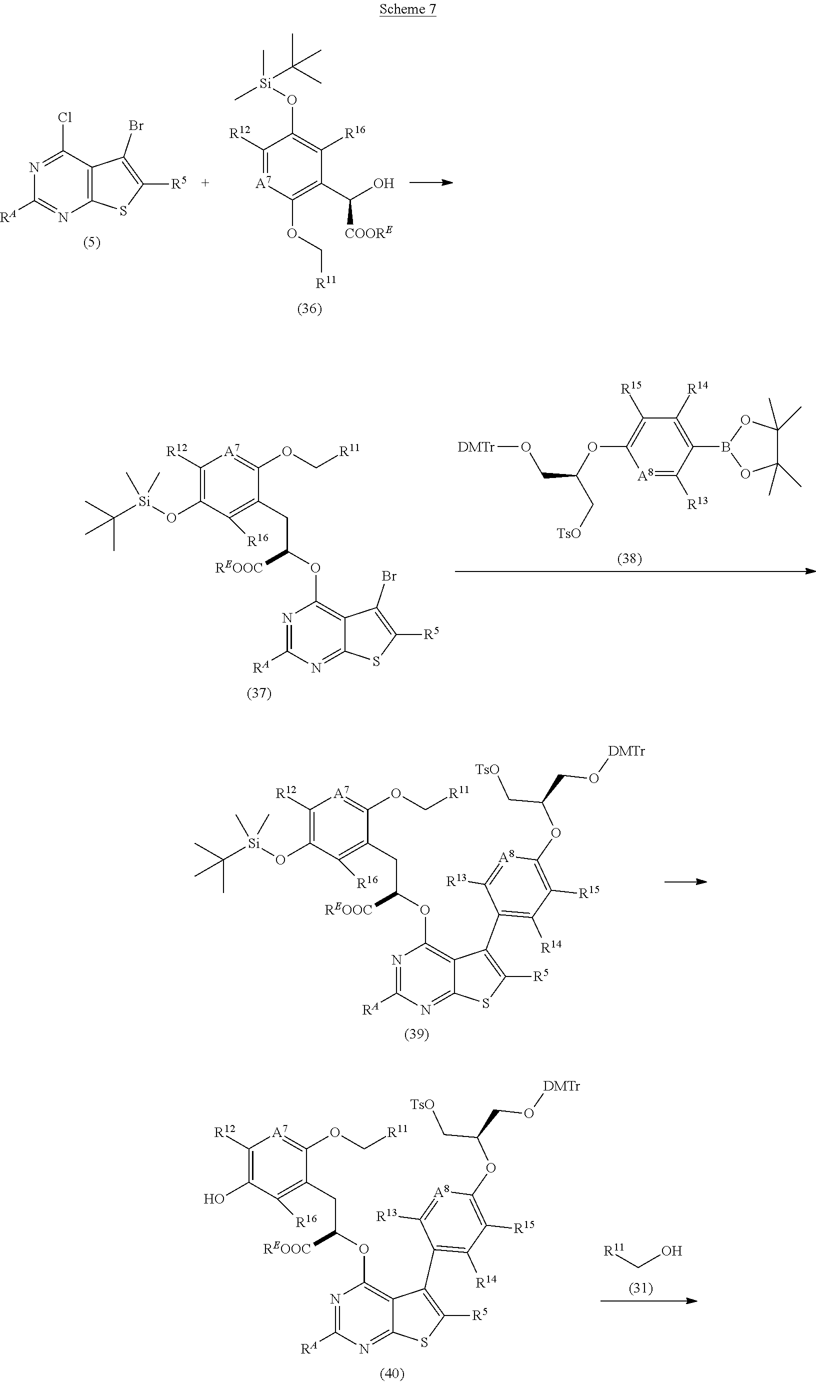

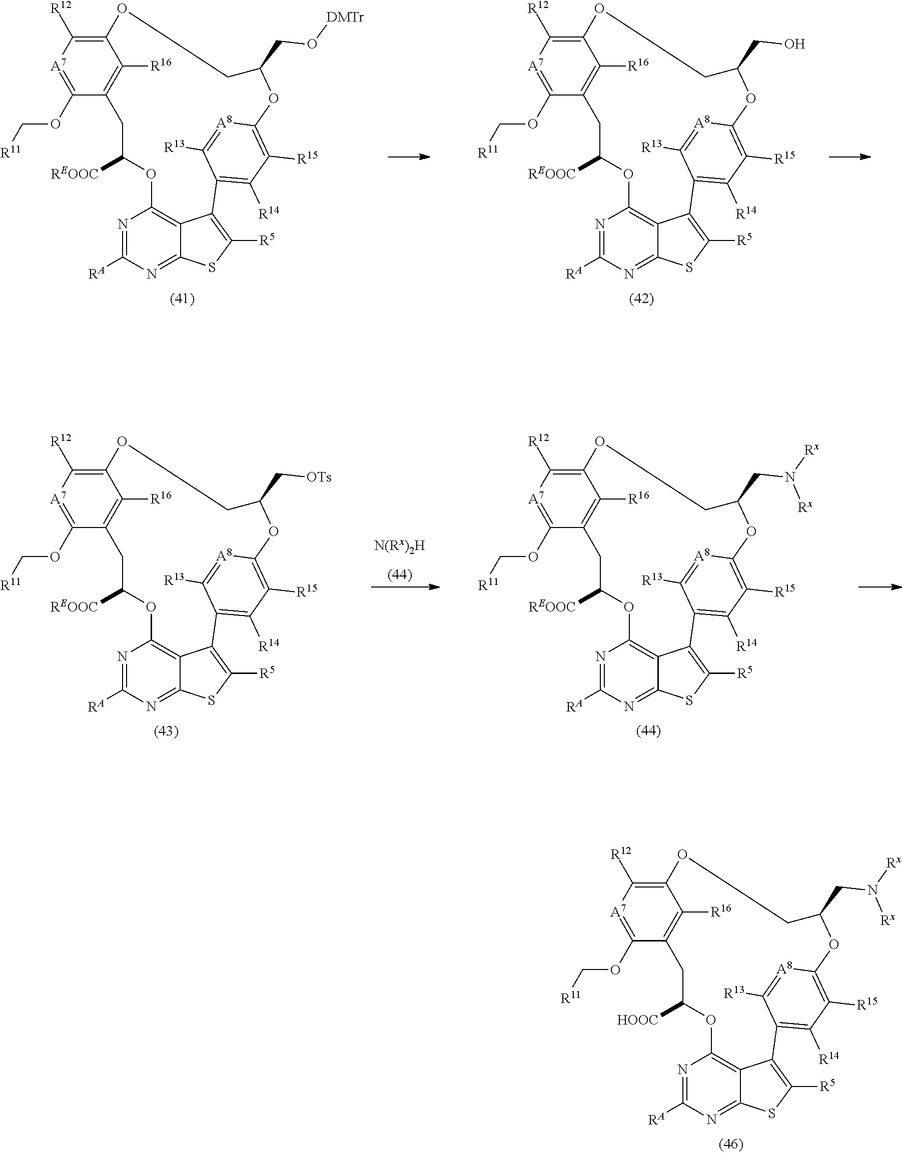

Scheme 7 describes the synthesis of macrocyclic compounds of the Formula (46), which are representative of compounds of Formula (I). Intermediates of the Formula (5) can be reacted with compounds of the Formula (36), wherein A⁷, R¹¹, R¹², R¹⁶ are as described herein and R^{E} is alkyl, in the presence of base such as, but not limited to, cesium carbonate, to provide compounds of the Formula (37). The reaction is typically conducted at an elevated temperature, such as, but not limited to 65° C., in a solvent such as but not limited to tert-butanol, N,N-dimethylformamide, or mixtures thereof. Compounds of Formula (39) can be prepared by reacting compounds of Formula (37) with a boronate ester (or the equivalent boronic acid) of Formula (38) under Suzuki Coupling conditions described herein or in the literature. Compounds of Formula (39) can be treated with tetrabutylammonium fluoride in a solvent system such as dichloromethane, tetrahydrofuran or mixtures thereof to provide compounds of Formula (40). Treatment of compounds of Formula (40) with a base such as, but not limited to, cesium carbonate in a solvent such as, but not limited to, N,N-dimethylformamide, will provide compounds of Formula (41). The reaction is typically performed at an elevated temperature, or more preferably at ambient temperature. Compounds of the Formula (41) can be deprotected to give compounds of the Formula (42) using procedures described herein or available in the literature. For example, compounds of Formula (41) can be treated with formic acid at ambient temperature in a solvent system such as, but not limited to, dichloromethane and methanol, to provide compounds of the Formula (42). Compounds of the Formula (42) can be treated with para-toluenesulfonyl chloride in the presence of a base such as, but not limited to, triethylamine or DABCO (1,4-diazabicyclo[2.2.2]octane) to provide compounds of Formula (43). The reaction is typically performed at low temperature before warming to room temperature in a solvent such as, but not limited to, dichloromethane. Compounds of Formula (43) can be reacted with amine nucleophiles of Formula (44), wherein two R^{x}, together with the nitrogen to which they are attached, optionally form a heterocycle, to provide intermediates of Formula (45). The reaction is typically performed in a solvent such as, but not limited to, N,N-dimethylformamide, at ambient temperature before heating to 35° C. to 40° C. Compounds of Formula (46) can be prepared by treating compounds of Formula (45) with lithium hydroxide. The reaction is typically performed at ambient temperature in a solvent such as, but not limited to, tetrahydrofuran, methanol, water, or mixtures thereof.

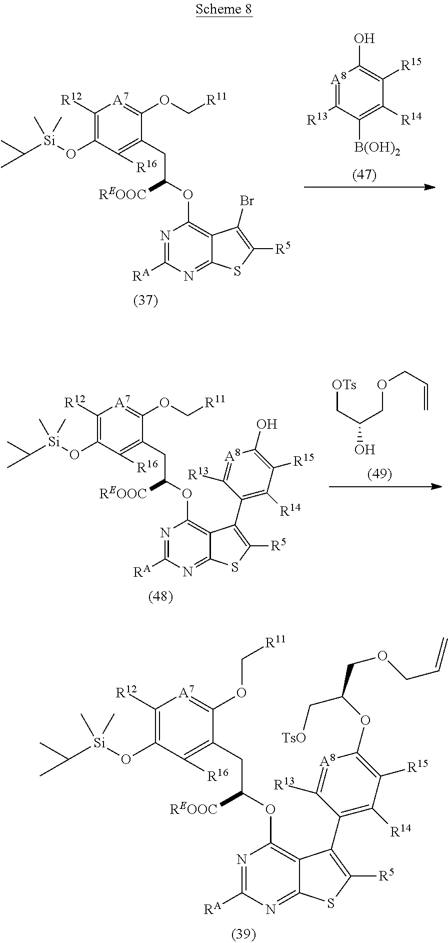

Scheme 8 describes an alternative synthesis of intermediates of the Formula (39). Compounds of Formula (48) can be prepared by reacting compounds of Formula (37) with a boronate ester (or the equivalent boronic acid) of Formula (47) under Suzuki Coupling conditions described herein or available in the literature. Compounds of the Formula (48) can be reacted with compounds of Formula (49) under Mitsunobu conditions described herein or available in the literature to provide compounds of the Formula (39). Compounds of the Formula (39) can be further treated as described in Scheme 7 or using methods described herein to provide macrocyclic compounds of the Formula (46), which are representative of compounds of Formula (I).

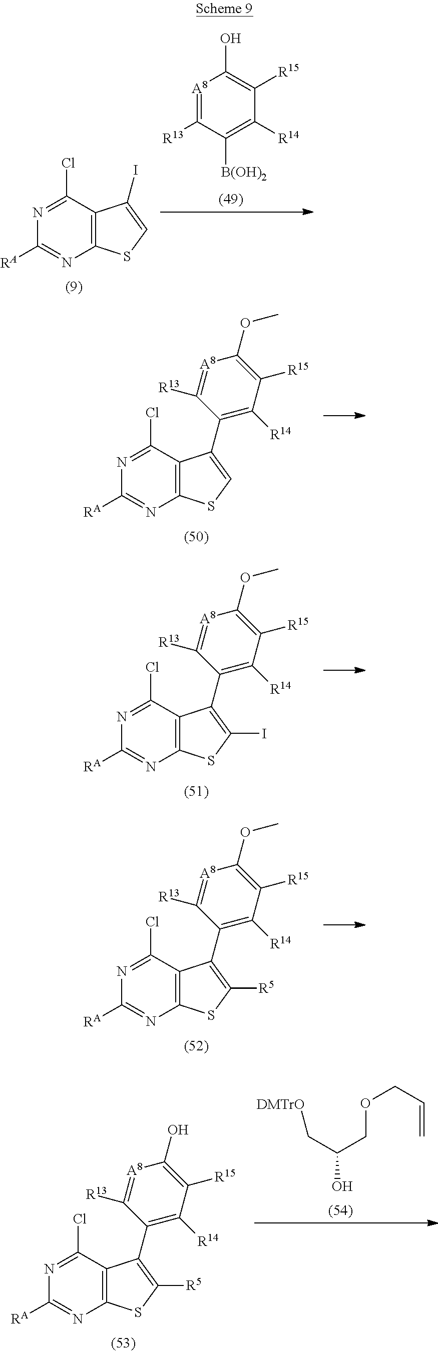

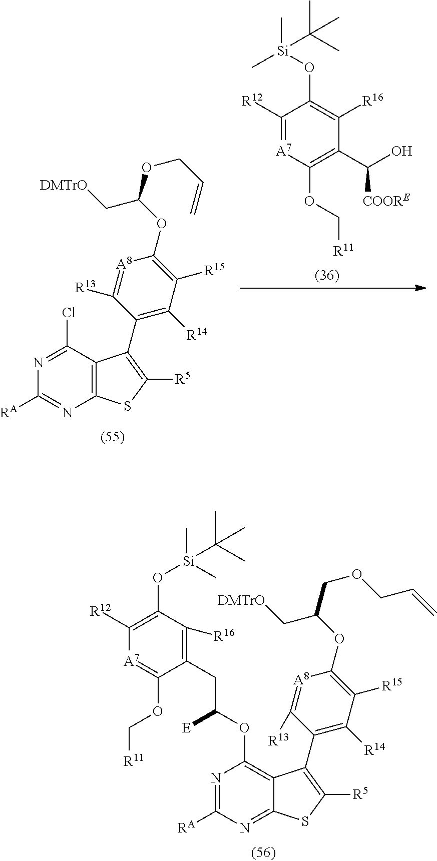

Scheme 9 describes the synthesis of compounds of Formula (56). Compounds of Formula (50) can be prepared by reacting compounds of Formula (9) with a boronate ester (or the equivalent boronic acid) of Formula (49) under Suzuki Coupling conditions described herein or available in the literature. Compounds of Formula (50) can be treated with a strong base such as, but not limited to lithium diisopropylamide, followed by the addition of iodine to provide compounds of the Formula (51). The reaction is typically performed in a solvent such as, but not limited to, tetrahydrofuran, at a reduced temperature before warming to ambient temperature. Compounds of Formula (52) can be prepared by reacting compounds of Formula (51) with a boronate ester (or the equivalent boronic acid) of Formula (6) under Suzuki Coupling conditions described herein or known in the literature. Compounds of Formula (52) can be treated with aluminum trichloride to provide compounds of Formula (53). The reaction is typically performed at an elevated temperature, for example from 60° C. to 70° C., in a solvent, such as but not limited to, 1,2-dichloroethane. Compounds of Formula (53) can be treated with compounds of Formula (54) under Mitsunobu conditions described herein or available in the literature to provide compounds of the Formula (55). Compounds of Formula (55) can be reacted with compounds of Formula (36) in the presence of a base such as, but not limited to, cesium carbonate to provide compounds of Formula (56). The reaction is typically performed at an elevated temperature in a solvent such as tert-butanol, N,N-dimethylformamide, or mixtures thereof. Compounds of Formula (56) can be used as described in subsequent steps herein to provide compounds of Formula (I).

It should be appreciated that the synthetic schemes and specific examples as illustrated in the synthetic examples section are illustrative and are not to be read as limiting the scope of the disclosure as it is defined in the appended claims. All alternatives, modifications, and equivalents of the synthetic methods and specific examples are included within the scope of the claims.

Optimum reaction conditions and reaction times for each individual step can vary depending on the particular reactants employed and substituents present in the reactants used. Specific procedures are provided in the Synthetic Examples section. Reactions can be worked up in the conventional manner, e.g. by eliminating the solvent from the residue and further purified according to methodologies generally known in the art such as, but not limited to, crystallization, distillation, extraction, trituration and chromatography. Unless otherwise described, the starting materials and reagents are either commercially available or can be prepared by one skilled in the art from commercially available materials using methods described in the chemical literature.

Manipulation of the reaction conditions, reagents and sequence of the synthetic route, protection of any chemical functionality that can not be compatible with the reaction conditions, and deprotection at a suitable point in the reaction sequence of the method are included in the scope of the present disclosure. Suitable protecting groups and the methods for protecting and deprotecting different substituents using such suitable protecting groups are well known to those skilled in the art; examples of which can be found in T. Greene and P. Wuts, Protecting Groups in Organic Synthesis (3^{rd} ed.), John Wiley & Sons, NY (1999), which is incorporated herein by reference in its entirety. Synthesis of the compounds of the present disclosure can be accomplished by methods analogous to those described in the synthetic schemes described hereinabove and in specific examples.

Starting materials, if not commercially available, can be prepared by procedures selected from standard organic chemical techniques, techniques that are analogous to the synthesis of known, structurally similar compounds, or techniques that are analogous to the above described schemes or the procedures described in the synthetic examples section.

When an optically active form of a compound is required, it can be obtained by carrying out one of the procedures described herein using an optically active starting material (prepared, for example, by asymmetric induction of a suitable reaction step), or by resolution of a mixture of the stereoisomers of the compound or intermediates using a standard procedure (such as chromatographic separation, recrystallization or enzymatic resolution).

Similarly, when a pure geometric isomer of a compound is required, it can be prepared by carrying out one of the above procedures using a pure geometric isomer as a starting material, or by resolution of a mixture of the geometric isomers of the compound or intermediates using a standard procedure such as chromatographic separation.

### Pharmaceutical Compositions

When employed as a pharmaceutical, a compound of the present disclosure may be administered in the form of a pharmaceutical composition. One embodiment pertains to a pharmaceutical composition comprising a therapeutically effective amount of a compound of Formula (I) according to claim 1, or a pharmaceutically acceptable salt thereof, in combination with a pharmaceutically acceptable carrier. The phrase “pharmaceutical composition” refers to a composition suitable for administration in medical or veterinary use.

The term “pharmaceutically acceptable carrier” as used herein, means a non-toxic, inert solid, semi-solid or liquid filler, diluent, encapsulating material or Formulation auxiliary.

### Methods of Use

The compounds of Formula (I), or pharmaceutically acceptable salts thereof, and pharmaceutical compositions comprising a compound of Formula (I), or a pharmaceutically acceptable salt thereof, may be administered to a subject suffering from a disorder or condition associated with MCL-1 overexpression or up-regulation. The term “administering” refers to the method of contacting a compound with a subject. Disorders or conditions associated with MCL-1 overexpression or up-regulation may be treated prophylactically, acutely, and chronically using compounds of Formula (I), depending on the nature of the disorder or condition. Typically, the host or subject in each of these methods is human, although other mammals may also benefit from the administration of a compound of Formula (I).

In embodiments, the present disclosure provides a method of treating a subject having cancer, wherein the method comprises the step of administering to the subject a therapeutically effective amount of a compound of Formula (I) or an embodiment thereof, with or without a pharmaceutically acceptable carrier. In embodiments, the cancer is an MCL-1 mediated disorder or condition. A “MCL-1-mediated disorder or condition” is characterized by the participation of MCL-1 in the inception and/or manifestation of one or more symptoms or disease markers, maintenance, severity, or progression of a disorder or condition. In embodiments, the present disclosure provides a method for treating multiple myeloma. The method comprises the step of administering to a subject in need thereof a therapeutically effective amount of a compound of Formula (I) or a preferred embodiment thereof, with or without a pharmaceutically acceptable carrier.

In embodiments, the present disclosure provides compounds of the disclosure, or pharmaceutical compositions comprising a compound of the disclosure, for use in medicine. In embodiments, the present disclosure provides compounds of the disclosure, or pharmaceutical compositions comprising a compound of the disclosure, for use in the treatment of diseases or disorders as described herein above.

One embodiment is directed to the use of a compound according to Formula (I), or a pharmaceutically acceptable salt thereof in the preparation of a medicament. The medicament optionally can comprise at least one additional therapeutic agent. In some embodiments the medicament is for use in the treatment of diseases and disorders as described herein above.

This disclosure is also directed to the use of a compound according to Formula (I), or a pharmaceutically acceptable salt thereof in the manufacture of a medicament for the treatment of the diseases and disorders as described herein above. The medicament optionally can comprise at least one additional therapeutic agent.

The compounds of Formula (I) may be administered as the sole active agent or may be co-administered with other therapeutic agents, including other compounds that demonstrate the same or a similar therapeutic activity and that are determined to be safe and efficacious for such combined administration. The term “co-administered” means the administration of two or more different therapeutic agents or treatments (e.g., radiation treatment) that are administered to a subject in a single pharmaceutical composition or in separate pharmaceutical compositions. Thus co-administration involves administration at the same time of a single pharmaceutical composition comprising two or more different therapeutic agents or administration of two or more different compositions to the same subject at the same or different times.

### EXAMPLES

The following Examples may be used for illustrative purposes and should not be deemed to narrow the scope of the present disclosure.

All reagents were of commercial grade and were used as received without further purification, unless otherwise stated. Commercially available anhydrous solvents were used for reactions conducted under inert atmosphere. Reagent grade solvents were used in all other cases, unless otherwise specified. Chemical shifts (δ) for ¹H NMR spectra were reported in parts per million (ppm) relative to tetramethylsilane (δ 0.00) or the appropriate residual solvent peak, i.e. CHCl₃ (δ 7.27), as internal reference. Multiplicities were given as singlet (s), doublet (d), triplet (t), quartet (q), quintuplet (quin), multiplet (m) and broad (br).

### Example 1

### (7R,20S)-18-chloro-1-(4-fluorophenyl)-10-{[2-(2-methoxyphenyl)pyrimidin-4-yl]methoxy}-19-methyl-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylic acid

### Example 1A

### 6-iodothieno[2,3-d]pyrimidin-4(3H)-one

Acetic acid (312 mL), sulfuric acid (9.37 mL) and water (63 mL) were combined with stirring. Thieno[2,3-d]pyrimidin-4(3H)-one (50 g), periodic acid (37.4 g) and iodine (75 g) were added sequentially, and the mixture became slightly endothermic. A heating mantle was added and the reaction mixture was ramped up to 60° C. Midway through, the temperature climbed to 68-69° C. The heating mantle was removed and the temperature was maintained at 70° C. by self-heating for about 45 minutes. LC/MS indicated a single peak corresponding to the title compound. The reaction mixture was cooled to room temperature. The resulting suspension was filtered, and washed with 5:1 acetic acid:water (three times), and diethyl ether (5×) to provide the title compound which was used in the next step without further purification. ¹H NMR (400 MHz, dimethyl sulfoxide-d₆) δ ppm 12.80-12.41 (m, 1H), 8.10 (s, 1H), 7.66 (s, 1H). MS (ESI) m/z 277.9 (M−H)⁻.

### Example 1B

### 4-chloro-6-iodothieno[2,3-d]pyrimidine

Phosphorous oxychloride (37 mL) and N,N-dimethylaniline (11.5 mL) were combined, and Example 1A (25 g) was added over a few minutes. The reaction mixture was stirred at about 105° C. for 1.5 hours. An aliquot was analyzed by LC/MS, which indicated the reaction mixture was complete. The suspension was cooled to 5-10° C., filtered, and washed with heptanes. The crude filter cake was dumped into ice water with rapid stirring. The mixture was stirred for about 30 minutes, filtered, and washed with additional water (3 times) and diethyl ether (3 times). The material was dried on the filter bed overnight to provide the title compound and was used in the next step without further purification. ¹H NMR (400 MHz, dimethyl sulfoxide-d₆) δ ppm 8.89 (s, 1H), 7.95 (s, 1H).

### Example 1C

### 5-bromo-4-chloro-6-iodothieno[2,3-d]pyrimidine

Example 1B (20.5 g) was taken up in acetonitrile (173 mL) and N-bromosuccinimide (13.54 g) was added followed by tetrafluoroboric acid-dimethyl ether complex (2 mL). While the reaction mixture was stirring, the temperature slowly climbed, reaching 25.5° C. after 30 minutes. The reaction mixture was allowed to stir overnight at room temperature. An additional 0.4 equivalents of N-bromosuccinimide was added followed by tetrafluoroboric acid-dimethyl ether complex (2 mL), and the reaction mixture was stirred for an additional 5 hours. The reaction mixture was cooled in an ice bath to about 5° C. (internal) and filtered. The material was washed with acetonitrile (twice) and dried on the filter bed overnight. The title compound was used in the next step without further purification. ¹H NMR (400 MHz, dimethyl sulfoxide-d₆) δ ppm 8.93 (s, 1H).

### Example 1D

### 5-bromo-4-chloro-6-(4-fluorophenyl)thieno[2,3-d]pyrimidine

(Tris(dibenzylideneacetone)dipalladium(0)) (7.32 g), di-tert-butyl(2′,4′,6′-triisopropyl-[1,1′-biphenyl]-2-yl)phosphine (7.47 g), tripotassium phosphate (181 g), (4-fluorophenyl)boronic acid (89 g), and Example 1C (200 g) were combined in a three neck, 5 L round bottom flask, fitted with a water condenser, thermocouple/JKEM, overhead stirring and an argon gas inlet. The material was flushed with argon for 40 minutes. Tetrahydrofuran (1705 mL) and water (426 mL) were combined into a 3 L round bottom flask. The contents were sparged with argon for 30 minutes. The solvent mixture was cannulated into the flask containing the material. A sharp temperature increase to 37° C. was observed. The temperature was set to 64° C. (internal), and the reaction mixture was stirred overnight (16 hours) at 64° C. under a light positive flow of argon. The reaction mixture was cooled to 38° C., and 200 mL water was added with stirring (overhead). Stirring was continued for 2 hours, and the material was filtered and washed with water. A second crop was obtained from the filtrate and was combined with the first crop. The combined material was taken up in hot tetrahydrofuran (2 L), stirred with 20 g thiosilica gel and 20 g charcoal for 30 minutes, and filtered through a pad of diatomaceous earth. The filtrate was concentrated to provide the title compound. ¹H NMR (400 MHz, chloroform-d) δ ppm 8.86 (s, 1H), 7.75-7.58 (m, 2H), 7.22 (t, 2H). MS (ESI) m/z 344.8 (M+H)⁺.

### Example 1E

### 2-methoxybenzimidamide hydrochloride

A dried 12 L five-necked flask equipped with a mechanical stirrer, a gas inlet with tubing leading to a nitrogen regulator, a gas inlet adapter with tubing leading to a bubbler, and an internal temperature probe (J-KEM controlled), was charged with ammonium chloride (86 g). The material was mixed under nitrogen with anhydrous toluene (2 L). The mixture was cooled to −12.3° C. in an ice/methanol bath. To the mixture was added, via cannula, 2.0 M trimethylaluminum in toluene (800 mL). Upon addition of the trimethylaluminum, the mixture started to smoke immediately and gas was evolved. The temperature of the reaction mixture rose to a high of −0.4° C. during the addition, and the addition took a total of about 60 minutes. After all the trimethylaluminum was added, the mixture was allowed to stir at 20° C. for 3 hours. To the mixture was added 2-methoxybenzonitrile (107 g) as a liquid (had been melted in bath at about 45° C.). Once the 2-methoxybenzonitrile was added, the reaction mixture was heated at 90° C. overnight with the use of a heating mantle controlled by a J-KEM. The reaction flask was fitted with a vigreux condenser. Thin-layer chromatography in 50% ethyl acetate/heptane indicated a major baseline product. The reaction mixture was cooled to −8.7° C. in an ice/methanol bath, and to the cold mixture was added 4 L of methanol, dropwise via an addition funnel. The addition evolved gas and was exothermic. The temperature of the reaction mixture reached a high of 7.9° C., and the addition took a total of about one hour. After all the methanol was added, the mixture was allowed to stir for three hours at 20° C. The reaction mixture was filtered through filter paper on a benchtop filter. The material collected were washed with additional methanol (2 L). The filtrate was concentrated. The crude material was mixed with 500 mL of ethyl acetate. The mixture was sonicated for 30 minutes and was stirred for another 30 minutes. The material was filtered off and washed with more ethyl acetate. The material collected was air dried for an hour and then dried under high vacuum for two hours to provide the title compound. ¹H NMR (400 MHz, dimethyl sulfoxide-d₆) δ ppm 9.23 (bs, 2H), 7.69 (bs, 1H), 7.63 (ddd, 1H), 7.55 (dd, 1H), 7.25 (dd, 1H), 7.12 (td, 1H), 3.87 (s, 3H). MS (DCI) m/z 151.0 (M+H)⁺.

### Example 1F

### 4-(dimethoxymethyl)-2-(2-methoxyphenyl)pyrimidine

An oven-dried 5 L three neck flask equipped with a mechanical stirrer, nitrogen inlet into a reflux condenser and outlet to a bubbler, and an internal temperature probe (J-KEM controlled), was charged with Example 1E (126.9 g) and (E)-4-(dimethylamino)-1,1-dimethoxybut-3-en-2-one (177 g). Anhydrous methanol (1360 mL) was added. To the mixture at room temperature under nitrogen was added solid sodium methoxide (257 g) in portions over 20 minutes. The temperature of the reaction went up from 18.6° C. to 35.7° C. during the addition. Once the exotherm stopped, the reaction mixture was heated to 65° C. overnight. The reaction mixture was cooled, and concentrated. The residue was mixed with ethyl acetate (800 mL), and water (1 L) was added carefully. The two phase mixture was sonicated for about 30 minutes to dissolve all the material. The layers were separated, and organic layer was washed with saturated aqueous NH₄Cl mixture. The combined aqueous extracts were extracted one time with ethyl acetate. The combined organic extracts were washed with brine, dried with Na₂SO₄, filtered, and concentrated. The residue was dissolved in a small amount of dichloromethane (30 mL) and loaded onto a 2.0 L plug of silica in a 3 L Buchner funnel that had been equilibrated with 40% ethyl acetate/heptane. The desired product was eluted with 40% to 50% ethyl acetate/heptane. The fractions containing the desired product were combined, and were concentrated to provide the title compound. ¹H NMR (500 MHz, dimethyl sulfoxide-d₆) δ ppm 8.93 (d, 1H), 7.54 (dd, 1H), 7.50-7.43 (m, 2H), 7.16 (dd, 1H), 7.06 (td, 1H), 5.31 (s, 1H), 3.76 (s, 3H), 3.38 (s, 6H). MS (DCI) m/z 261.0 (M+H)⁺.

### Example 1G

### (2-(2-methoxyphenyl)pyrimidin-4-yl)methanol

A mixture of Example 1F (14.7 g) in 110 mL HCl in dioxane (4M mixture) and 110 mL water was heated at 50° C. for 14 hours. The mixture was cooled to 0° C., and ground NaOH (17.60 g) was added in portions. The pH was adjusted to 8 using 10% K₂CO₃ aqueous mixture. NaBH₄ (4.27 g) was added in portions. The mixture was stirred at 0° C. for 45 minutes. The mixture was carefully quenched with 150 mL saturated aqueous NH₄Cl and was stirred at 0° C. for 30 minutes. The mixture was extracted with ethyl acetate (5×150 mL), washed with brine, dried over MgSO₄, filtered, and concentrated. The residue was triturated in 30 mL ethanol to give a first crop of the title compound. The filtrate was concentrated and the residue was purified on a silica gel column (120 g, 55-100% ethyl acetate in heptanes, dry load) to give a second crop of the title compound. ¹H NMR (500 MHz, dimethyl sulfoxide-d₆) δ ppm 8.84 (d, 1H), 7.49 (m, 2H), 7.44 (ddd, 1H), 7.13 (dd, 1H), 7.04 (td, 1H), 5.65 (t, 1H), 4.60 (dd, 2H), 3.75 (s, 3H). MS (DCI) m/z 217.0 (M+H)⁺.

### Example 1H

### ethyl 2-acetoxy-3-(2-(benzyloxy)phenyl)acrylate

A 2 L three-necked round bottom flask equipped with an internal temperature probe was charged with ethyl 2-acetoxy-2-(diethoxyphosphoryl)acetate (86 g) and anhydrous tetrahydrofuran (1 L) at room temperature under nitrogen gas. To the mixture was added cesium carbonate (100 g, 307 mmol) in one portion. The reaction mixture was stirred for about 20 minutes, and 2-(benzyloxy)benzaldehyde (50 g) was added in one portion. The slurry was stirred vigorously overnight at room temperature. Thin-layer chromatography in 10% ethyl acetate/heptane indicted the reaction was about 60 to 70% complete. Another 0.5 equiv of ethyl 2-acetoxy-2-(diethoxyphosphoryl)acetate and cesium carbonate were added, and the reaction mixture was stirred overnight. Thin-layer chromatography indicated the reaction mixture was complete. The reaction mixture was cooled to about 0° C. in an ice bath, and the reaction mixture was quenched with water (500 mL) in portions. Water was added such that the temperature of the reaction mixture was maintained below 10° C. The reaction mixture was diluted with ethyl acetate (500 mL), and the mixture was stirred for 30 minutes. The mixture was poured into a separatory funnel and was further diluted with ethyl acetate and water to a total volume of 2.6 L. The organic layer was separated, washed with brine, dried with Na₂SO₄, filtered, and concentrated. The residue was dissolved in 2:1 heptane/dichloromethane and was purified on a 2 L silica gel plug equilibrated with 100% heptane. The material was eluted with 5% to 10% ethyl acetate/heptane. The pure fractions were combined, and the solvents were removed under reduced pressure to provide the title compound. NMR indicated the material was about a 2:1 mix of E and Z isomer. ¹H NMR (501 MHz, dimethyl sulfoxide-d₆) δ ppm 7.71 (m, 2H), 7.50-7.25 (m, 12H), 7.20 (dd, 1H), 7.11 (dd, 0.5H), 7.04 (m, 1H), 6.94 (m, 1H), 5.22 (s, 2H), 5.14 (s, 1H), 4.20 (q, 2H), 4.01 (q, 1H), 2.30 (s, 3H), 2.21 (s, 1.5H), 1.24 (t, 3H), 0.99 (t, 1.5H). MS (ESI) m/z 340.8 (M+H)⁺.

### Example 1I

### (R)-ethyl 2-acetoxy-3-(2-(benzyloxy)phenyl)propanoate

Example 1H (1.0 kg) in methanol (5.0 L) was degassed with bubbling argon for 30 minutes and then transferred to a 2 gallon Parr stainless steel reactor. The reactor was purged with argon for 30 minutes. At that time, 1,2-bis((2R,5R)-2,5-diethylphospholano)benzene(cyclooctadiene)rhodium(I) tetrafluoroborate (17.8 g) was added, and the vessel was sealed and purged further with argon. The vessel was pressurized to 120 psi with hydrogen. The mixture was stirred under 120 psi of hydrogen with no external heating applied. After 70 hours, the reactor was vented and purged 4 times with argon. HPLC indicated complete conversion to the desired product. The mixture was transferred to a flask, and the solvents were concentrated. To the residue was added 1:1 heptane/ethyl acetate, and the clear material turned into a cloudy mix. The flask was swirled, and a sludge crashed out. With the swirling, much of the sludge stuck to the side of the flask. The material was poured through a plug of silica (1 L), eluting with 1:1 heptane/ethyl acetate. The filtrate which contained the title compound was concentrated to provide the title compound. ¹H NMR (400 MHz, Chloroform-d) δ ppm 7.47 (m, 2H), 7.39 (m, 2H), 7.32 (m, 1H), 7.19 (m, 2H), 6.90 (m, 2H), 5.31 (dd, 1H), 5.12 (m, 2H), 4.13 (qq, 2H), 3.35 (dd, 1H), 3.06 (dd, J=13.8, 9.2 Hz, 1H), 2.03 (s, 3H), 1.17 (t, 3H). MS (ESI) m/z 360.0 (M+NH₄)⁺.

### Example 1J

### (R)-ethyl 2-acetoxy-3-(2-hydroxyphenyl)propanoate

Example 1I (896 g) in ethanol (4.3 L) was added to wet 5% palladium on carbon catalyst (399.7 g) in a 2 gallon Parr stainless steel reactor. The reactor was purged with argon, and the mixture was stirred at 600 RPM under 50 psi of hydrogen at 25° C. for 12 hours. LC/MS indicated a single peak corresponding to the title compound. The mixture was filtered through filter paper and followed by a 0.2 micron polypropylene membrane. The mixture was concentrated to produce an material that formed a precipiate upon standing overnight. The precipiate were transferred into a 12 L three-neck round bottom flask equipped with a mechanical stirrer and temperature probe (J-KEM controlled). The material was mixed in 5 L (about 0.5M) of heptane. The mixture was heated to about 74° C. To the hot mixture was added isopropyl acetate. The isopropyl acetate was added in 100 mL aliquots up to about 500 mL. The material was almost all dissolved. Isopropyl acetate was added in 10 mL aliquots until a clear, mixture formed. A total of 630 mL of isopropyl acetate was used. The mixture was heated to about 80° C. for about 10 minutes. The heat was turned off but the heating mantle was left on. Stirring was slowed to a low rate. The mixture was allowed to cool slowly overnight. The mixture was filtered, and the material was washed with heptane, and dried for a few hours. The filtrate was concentrated, and the process was repeated on the residue using the same conditions to produce additional title compound. The two batches of title compound were combined. Chiral HPLC of the combined material on a Gilson HPLC system using a ChiralPak AD-H column (4.6 mm×250 mm, 3 uM) and a 5% to 50% ethanol/heptane gradient over 15 minutes indicated a single peak with a retention time of 8.9 minutes. ¹H NMR (400 MHz, dimethyl sulfoxide-d₆) δ ppm 9.53 (s, 1H), 7.06 (m, 2H), 6.79 (m, 1H), 6.71 (td, 1H), 5.11 (dd, J=8.3, 6.0 Hz, 1H), 4.05 (q, 2H), 3.07 (dd, 1H), 2.95 (dd, 1H), 2.00 (s, 3H), 1.09 (t, 3H). MS (DCI) m/z 270.0 (M+NH₄)⁺.

### Example 1K

### (R)-ethyl 2-acetoxy-3-(5-bromo-2-hydroxyphenyl)propanoate

A dried 5 L three neck jacketed flask equipped with a mechanical stirrer and an internal temperature probe controlled by a Huber Ministat 230 chiller was charged with Example 1J (200 g). To this was added anhydrous tetrahydrofuran (3.3 L) at room temperature under nitrogen. The mixture was cooled to −20.4° C. using a chiller. To the cooled mixture was added concentrated sulfuric acid (4.23 mL). The temperature of the reaction rose to −19.8° C. N-Bromosuccinimide (143 g) was added in portions over a period of 10 minutes. The temperature rose from −20.3° C. to −20.0° C. during the addition. The reaction mixture was stirred overnight at −20° C. LC/MS indicated the reaction mixture was about 70% complete. The reaction mixture was warmed to 0° C. with the use of the chiller and was stirred for 5 hours at 0° C. LC/MS indicated reaction mixture was greater than 90% complete. The reaction mixture was warmed to 20° C. with use of the chiller. After one hour at 20° C., LC/MS showed no sign of starting material and one major product. The reaction mixture was cooled to 0° C. with use of the chiller. The reaction mixture was quenched with 500 mL of water, and the temperature rose from 0° C. to about 8° C. The reaction mixture was diluted with ethyl acetate (1.0 L), and two-phase mixture was stirred for about 20 minutes. The two phase mixture was poured into a 6 L separatory funnel. One liter of water was added, the mixture shaken, and the layers were separated. The organic layer was washed with saturated aqueous NaHCO₃ mixture and brine. The combined aqueous layers were back-extracted one time with ethyl acetate. The combined organic extracts were dried with Na₂SO₄, filtered, and concentrated. Dichloromethane (300 mL) was added to the residue. The mixture was sonicated for 60 minutes. The material was filtered, washed with a minimum amount of dichloromethane, and dried for an hour to provide the title compound. The material that formed in the filtrate were filtered and washed with ethyl acetate. The two batches of material were combined and dried in a vacuum oven at 50° C. for 5 hours to provide the title compound. Chiral HPLC of this material on a Gilson HPLC system using a ChiralPak AD-H column (4.6 mm×250 mm, 3 μM) and a 5-50% ethanol/heptane gradient over 30 minutes indicated a single peak with a retention time of 10.6 minutes. ¹H NMR (400 MHz, dimethyl sulfoxide-d₆) δ ppm 9.89 (s, 1H), 7.22 (m, 2H), 6.76 (dt, 1H), 5.11 (dd, 1H), 4.06 (qq, 2H), 3.05 (dd, 1H), 2.97 (dd, 1H), 2.02 (s, 3H), 1.10 (t, 3H). MS (ESI) m/z 332.8 (M+H)⁺.

### Example 1L

### (R)-ethyl 2-acetoxy-3-(5-bromo-2-((2-(2-methoxyphenyl)pyrimidin-4-yl)methoxy)phenyl)propanoate

A 2 L three neck round bottom flask equipped with a temperature probe (J-KEM controlled) and stir bar was charged with Example 1K (40 g) and Example 1G (31.3 g) under nitrogen. The material was dissolved in anhydrous tetrahydrofuran (604 mL) at room temperature, and the reaction mixture was cooled to 2.3° C. in an ice bath. To the mixture was added triphenylphosphine (63.4 g). After about 15 minutes, (E)-N¹,N,N²,N²-tetramethyldiazene-1,2-dicarboxamide (41.6 g) was added in one portion. The temperature of the reaction did not rise significantly (temperature maintained at 2.5° C.). The reaction mixture was stirred at room temperature overnight. Thin-layer chromatography in 50% ethyl acetate/heptane indicated the starting materials were consumed, and a single major product had formed. The reaction mixture was filtered through a fritted Buchner funnel, and the material collected were washed with ethyl acetate. The filtrate was concentrated. The residue was dissolved in dichloromethane (150 mL), and loaded on to 2.2 L of silica gel that had been equilibrated in 30% ethyl acetate/heptane in a 3 L fritted Buchner funnel. The title compound was eluted with a gradient of 30-60% ethyl acetate in heptane. The early fractions were pure, but the later fractions were contaminated with triphenylphosphine oxide. The pure fractions were combined and were concentrated to provide the title compound. The impure fractions were combined and concentrated. The residue was dissolved in dichlormethane (50 mL) and purified on a Grace Reveleris X2 MPLC using a Teledyne Isco RediSep® Rf gold 750 g silica gel column, eluting with 30-50% ethyl acetate/heptane. Pure fractions from this column were combined with the pure material from the earlier column. The material that resulted was mixed with diethyl ether (50 mL). The mixture was sonicated for 30 minutes and stirred for an additional 10 minutes. The material was filtered off, washed with diethyl ether, and dried to provide the title compound. Chiral SFC of this material on a HP/Aurora system using a ChiralCel OD-H column (4.6 mm×100 mm, 5 μM) and a 5% to 50% methanol gradient over 10 minutes indicated a single peak with a retention time of 5.0 minutes. ¹H NMR (400 MHz, dimethyl sulfoxide-d₆) δ ppm 8.94 (d, 1H), 7.55 (m, 2H), 7.45 (m, 3H), 7.16 (m, 1H), 7.06 (m, 2H), 5.27 (d, 2H), 5.18 (dd, 1H), 4.07 (q, 2H), 3.77 (s, 3H), 3.29 (dd, 1H), 3.13 (dd, 1H), 2.02 (s, 3H), 1.10 (t, 3H). MS (ESI) m/z 529.1 (M+H)⁺.

### Example 1M

### (R,E)-ethyl 2-acetoxy-3-(5-(hex-1-en-1-yl)-2-((2-(2-methoxyphenyl)pyrimidin-4-yl)methoxy)phenyl)propanoate

A 1 L three neck round bottom flask equipped with a stir bar and an internal temperature probe (J-KEM controlled) was charged with Example 1L (41 g), ((E)-hex-1-en-1-ylboronic acid (19.82 g), palladium(II) acetate (1.74 g), dicyclohexyl(2′,6′-dimethoxy-[1,1′-biphenyl]-2-yl)phosphine (SPhos) (4.45 g), and CsF (35.3 g). The flask was sealed with septa, and the material was sparged for 60 minutes by blowing nitrogen over the material while stirring. Meanwhile in a separate 500 mL round bottom flask was added anhydrous 1,4-dioxane (620 mL), and the mixture was sparged subsurface with nitrogen for 60 minutes. The sparged solvent was then transferred via cannula to the flask with the material, and the reaction was stirred at room temperature. The temperature rose steadily and slowly from about 17.4° C. to about 33° C. The temperature started to go down after about 5 minutes once the high temperature was reached. LC/MS of the reaction mixture after 30 minutes at room temperature produced a single peak that corresponded to the desired product. The reaction mixture was diluted with ethyl acetate and water, and the two-phased mixture was stirred for about 30 minutes with about 3.8 g (˜3.0 equiv. based on moles of palladium) of APDTC (ammonium pyrrolidine dithiocarbamate) palladium scavenger. The mixture was filtered through diatomaceous earth with ethyl acetate washes. The filtrate was poured into a separatory funnel, and the layers were separated. The organic layer was washed with brine. The combined aqueous layers were back extracted one time with ethyl acetate. The combined organic layers were dried with Na₂SO₄, filtered, and concentrated. The residue was purified on a Grace Reveleris X2 MPLC using a Teledyne Isco RediSep® Rf gold 750 g silica gel column, eluting with 30% to 40% ethyl acetate/heptane. The product containing fractions were combined, and the solvents were concentrated to provide the title compound. ¹H NMR (400 MHz, dimethyl sulfoxide-d₆) δ ppm 8.93 (d, 1H), 7.55 (m, 2H), 7.47 (ddd, 1H), 7.25 (m, 2H), 7.16 (dd, 1H), 7.05 (m, 2H), 6.31 (m, 1H), 6.14 (dt, 1H), 5.26 (d, 2H), 5.18 (dd, 1H), 4.07 (q, 2H), 3.77 (s, 3H), 3.28 (dd, 1H), 3.11 (dd, 1H), 2.16 (m, 2H), 2.01 (s, 3H), 1.37 (m, 4H), 1.09 (t, 3H), 0.89 (t, 3H). MS (ESI) m/z 533.3 (M+H)⁺.

### Example 1N

### (R)-ethyl 2-acetoxy-3-(5-formyl-2-((2-(2-methoxyphenyl)pyrimidin-4-yl)methoxy)phenyl)propanoate

A 2 L three neck round bottom flask equipped with a stir bar and an internal temperature probe (J-KEM controlled) was charged with Example 1M (41 g) and iodobenzene diacetate (57.0 g). Tetrahydrofuran (733 mL) and water (36.7 mL) were added. To the mixture was added 2,6-lutidine (22.41 mL), followed by addition of solid osmium tetroxide (249 mg). The temperature of the reaction rose from 19.7° C. to 33° C. LC/MS of the mixture after 5 minutes indicated a single product had formed that corresponded to desired product. The reaction mixture was quenched with saturated aqueous sodium thiosulfate (500 mL), and was diluted further with ethyl acetate. The mixture was poured into a separatory funnel, and the layers were separated. The organic layer was washed with aqueous sodium thiosulfate and brine, and the washes were combined with the first thiosulfate wash. The combined thiosulfate washes were back extracted with dichloromethane, and the dichloromethane extract was combined with the original organic extract. The combined organic extracts were then washed with an aqueous copper sulfate mixture (twice) and brine. The organic extracts were dried with Na₂SO₄, filtered, and concentrated. The residue was purified on a Grace Reveleris X2 MPLC using a Teledyne Isco RediSep® Rf gold 750 g silica gel column eluting with 50% to 60% ethyl acetate/heptane. The product containing fractions were combined, and concentrated. The residue was dissolved in dichloromethane, and the mixture was loaded onto a plug of silica gel (300 mL-dry loaded) in a 500 mL plastic disposable Buchner funnel. The desired product was eluted with 50% to 60% to 70% ethyl acetate/heptane. The pure fractions were combined and concentrated to provide the title compound. Chiral HPLC on a Gilson HPLC system using a CHIRALCEL OD-H column (4.6 mm×250 mm, 5 μM) and a 20% to 100% ethanol/heptane gradient over 30 minutes indicated a single peak with a retention time of 29.0 minutes. ¹H NMR (400 MHz, dimethyl sulfoxide-d₆) δ ppm 9.89 (s, 1H), 8.95 (d, 1H), 7.87 (dd, 1H), 7.80 (d, 1H), 7.57 (m, 2H), 7.47 (ddd, 1H), 7.32 (d, 1H), 7.16 (dd, 1H), 7.06 (td, 1H), 5.42 (m, 2H), 5.22 (dd, 1H), 4.07 (q, 2H), 3.77 (s, 3H), 3.38 (dd, 1H), 3.22 (dd, 1H), 2.00 (s, 3H), 1.09 (t, 3H). MS (ESI) m/z 479.3 (M+H)⁺.

### Example 1O

### (R)-ethyl 3-(5-formyl-2-((2-(2-methoxyphenyl)pyrimidin-4-yl)methoxy)phenyl)-2-hydroxypropanoate

A 500 mL round bottom flask was charged with Example 1N (14.7 g). The material was mixed with anhydrous ethanol (219 mL). To the mixture at room temperature was added a 21% sodium ethoxide mixture in ethanol (0.573 mL). The reaction mixture was stirred for 3 hours at room temperature. LC/MS indicated a single product had formed that corresponded to the desired product. The reaction mixture was quenched with acetic acid (0.352 mL,), and was concentrated. The residue was dissolved in dichloromethane and loaded onto a plug of silica gel (300 mL-dry loaded) in a 500 mL plastic disposable fritted Buchner funnel. The desired product was eluted with 50% to 60% to 70% ethyl acetate/heptane. The desired product containing fractions were combined, and concentrated to provide the title compound. Chiral HPLC on a Gilson HPLC system using a ChiralCel OD-H column (4.6 mm×250 mm, 5 μM) and a 10% to 100% ethanol/heptane gradient over 20 minutes indicated a single peak with a retention time of 19.2 minutes. ¹H NMR (400 MHz, dimethyl sulfoxide-d₆) δ ppm 9.88 (s, 1H), 8.94 (d, 1H), 7.80 (m, 2H), 7.58 (m, 2H), 7.47 (ddd, 1H), 7.29 (d, 1H), 7.17 (dd, 1H), 7.06 (td, 1H), 5.61 (d, 1H), 5.40 (d, 2H), 4.39 (ddd, 1H), 4.07 (q, 2H), 3.77 (s, 3H), 3.23 (dd, 1H), 2.95 (dd, 1H), 1.12 (t, 3H). MS (ESI) m/z 437.2 (M+H)⁺.

### Example 10P

### (R)-ethyl 2-((5-bromo-6-(4-fluorophenyl)thieno[2,3-d]pyrimidin-4-yl)oxy)-3-(5-formyl-2-((2-(2-methoxyphenyl)pyrimidin-4-yl)methoxy)phenyl)propanoate

A 500 mL round bottom flask equipped with a stir bar and temperature probe (J-KEM controlled) was charged with Example 1O (9.2 g) and Example 1D (7.60 g). Anhydrous tert-butanol (162 mL) was added. The mixture was stirred to form a slurry. To the slurry was added cesium carbonate (27.5 g), and the mixture was heated to 65° C. After 4 hours of heating, thin-layer chromatography in 50% ethyl acetate/heptane indicated one major product with no starting material remaining. The reaction mixture was poured into a combination of saturated aqueous NH₄Cl, brine, and water. The flask was rinsed with ethyl acetate, and more ethyl acetate was added to the aqueous quench. Methanol was added to dissolve most of the material. The layers were separated, and aqueous layer was extracted one more time with 10% methanol/ethyl acetate. The combined organic extracts were washed with brine, dried with Na₂SO₄, filtered, and concentrated. The residue was dissolved in dichloromethane and was purified on a Grace Reveleris X2 MPLC using a Teledyne Isco RediSep® Rf gold 330 g silica gel column, eluting with 50-70% ethyl acetate in heptane. The pure fractions were collected, and the column was washed with 50-70% ethyl acetate/dichloromethane. The impure fractions were collected from the wash, and they were combined and concentrated. The crude material were purified on a Grace Reveleris X2 MPLC using a Teledyne Isco RediSep® Rf gold 220 g silica gel column eluting with 10-30% ethyl acetate/dichloromethane. The product containing fractions from both columns were combined to provide the title compound. ¹H NMR (400 MHz, dimethyl sulfoxide-d₆) δ ppm 9.89 (s, 1H), 8.92 (d, 1H), 8.60 (s, 1H), 8.06 (d, 1H), 7.86 (dd, 1H), 7.73 (m, 2H), 7.61 (d, 1H), 7.44 (m, 4H), 7.33 (d, 1H), 7.11 (d, 1H), 6.99 (t, 1H), 5.78 (dd, 1H), 5.42 (m, 2H), 4.17 (q, 2H), 3.75 (s, 3H), 3.66 (dd, 1H), 3.40 (m, 1H), 1.15 (t, 3H). MS (ESI) m/z 743.2 (M+H)⁺.

### Example 1Q

### 2-(4-bromo-2-chlorophenyl)-1,3-dioxane

A 3 L, three neck round bottom flask equipped with a Dean-Stark trap and reflux condenser was charged with 4-bromo-2-chlorobenzaldehyde (200 g), toluene (1519 mL), propane-1,3-diol (110 mL) and p-toluenesulfonic acid monohydrate (1.1 g). The reaction mixture was heated to reflux (112° C. internal) under Dean-Stark conditions, producing 18 mL of water in about 2 hours. The reaction mixture was cooled to room temperature and poured into saturated aqueous sodium bicarbonate mixture (600 mL) and ethyl acetate (500 mL). The layers were separated, and the aqueous layer was extracted with ethyl acetate (500 mL, once). The combined organics were dried (anhydrous MgSO₄) and treated with charcoal with stirring overnight. The mixture was filtered through a plug of diatomaceous earth and the filtrate was concentrated by rotary evaporation to provide the title compound. The title compound was placed in a vacuum oven overnight at 50° C. and was used in the next step without further purification. ¹H NMR (400 MHz, chloroform-d) δ ppm 7.57 (d, 1H), 7.51 (d, 1H), 7.42 (dd, 1H), 5.74 (s, 1H), 4.29-4.19 (m, 2H), 4.05-3.91 (m, 2H), 2.31-2.13 (m, 1H), 1.43 (dtt, 1H).

### Example 1R

### 2-(4-bromo-2-chloro-3-methylphenyl)-1,3-dioxane

A 5-neck, 5 L round bottom reactor was equipped with overhead stirring, thermocouple/JKEM, addition funnels and nitrogen inlet. The assembled reactor was dried with a heat gun under nitrogen. N,N-Diisopropylamine (138 mL) and tetrahydrofuran (1759 mL) were added to the reactor under a flow of nitrogen. The mixture was cooled to about −76° C. (internal) and n-butyllithium (369 mL, 923 mmol) was added via addition funnel at a rate necessary to keep the temperature below −68° C. The mixture was stirred at −76° C. for 45 minutes to generate a mixture of lithium diisopropylamide (LDA). A tetrahydrofuran (500 mL) mixture of Example 1Q (244.08 g) was added dropwise via addition funnel (over 45 minutes) to the LDA mixture at a rate necessary to keep the temperature below −68° C. The mixture was stirred for 2 hours at −76° C. Iodomethane (57.7 mL) was added dropwise over 1 hour via addition funnel (very exothrmic), and the temperature was kept below-70° C. during the addition. The reaction mixture was allowed to warm slowly to room temperature and was stirred overnight. In the morning, water and saturated aqueous ammonium chloride were added along with ethyl acetate (1 L). The layers were separated by pump, and the aqueous layer was extracted with ethyl acetate (twice) pumping the top layer into a seperatory funnel. The combined organics were dried (anhydrous MgSO₄), filtered through diatomaceous earth, and concentrated by rotary evaporation to provide the title compound. GC-MS indicated 11.71 minutes (3%, starting material), 12.82 minutes (8.2%, +Me) and product at 12.5 minutes (88.8%). The material (246 g) was slurried in 550 mL isopropyl alcohol. The mixture was heated to about 80° C. With stirring, the mixture was allowed to cool slowly to room temperature. Copious amounts of material formed, and the flask was placed in the freezer (−16° C.). After 1 hour, the material was broken up and 400 mL of ice cold isopropylachohol was added. The mixture was slurried and filtered through paper, washing quickly with cold isopropyl alcohol. The material was allowed to dry on the filter bed and was placed in the vacuum oven for 5 hours (50° C.) to provide the title compound. ¹H NMR (400 MHz, Chloroform-d) δ ppm 7.50 (d, 1H), 7.41 (d, 1H), 5.77 (s, 1H), 4.25 (ddd, 2H), 4.01 (td, 2H), 2.53 (s, 3H), 2.34-2.13 (m, 1H), 1.44 (ddt, 1H). MS (ESI) m/z 308.0 (M+NH₄)⁺.

### Example 1S

### 2-(3-chloro-4-(1,3-dioxan-2-yl)-2-methylphenyl)-4,4,5,5-tetramethyl-1,3,2-dioxaborolane

A 3-neck, 5 L round bottom flask fitted with a thermocouple/JKEM, dry ice acetone bath, overhead stirring, nitrogen inlet and outlets and addition funnel was charged with Example 1R (100 g) and tetrahydrofuran (1715 mL) under a positive flow of nitrogen. The mixture was cooled to −76° C. (internal) and n-butyllithium (151 mL, 377 mmol) was added dropwise via addition funnel, observing a temperature increase of 5-8° C. The mixture remained clear and colorless and was stirred for 10 minutes at −76° C. 2-Isopropoxy-4,4,5,5-tetramethyl-1,3,2-dioxaborolane (84 mL) was added dropwise (mixture became exothermic) at such a rate to keep the temperature below −68° C. The mixture was stirred at −76° C. for about 30 minutes, warmed to room temperature, and stirred for 3 hours. The reaction mixture was deemed complete by thin-layer chromatography (3:1 heptanes:ethyl acetate). The reaction mixture was concentrated by rotary evaporation. After the volatiles were removed, the water bath was set to 80° C., and the evaporator was switched to high vacuum for 1 hour. Water and ethyl acetate were added to the residue, and the layers were separated. The aqueous layer was extracted with ethyl acetate (once), and the combined organics were dried (anhydrous MgSO₄), filtered and concentrated. The material was triturated with ice-cold methanol, filtered through paper, and dried on the filter bed and vacuum oven (50° C.) to provide the title compound. ¹H NMR (400 MHz, dimethyl sulfoxide-d₆) δ ppm 7.59 (d, 1H), 7.45 (d, 1H), 5.76 (s, 1H), 4.14 (ddd, 2H), 3.96 (td, 2H), 2.53 (s, 2H), 2.09-1.94 (m, 1H), 1.50-1.39 (m, 1H), 1.31 (s, 9H). MS (ESI) m/z 339.3 (M+H)⁺.

### Example 1T

### (R)-ethyl 2-((5-((1S)-3-chloro-4-(1,3-dioxan-2-yl)-2-methylphenyl)-6-(4-fluorophenyl)thieno[2,3-d]pyrimidin-4-yl)oxy)-3-(5-formyl-2-((2-(2-methoxyphenyl)pyrimidin-4-yl)methoxy)phenyl)propanoate

A 500 mL round bottom flask was charged with Example 1P (8.9 g, 11.97 mmol), Example 1S (4.86 g), potassium phosphate (7.62 g), and bis(di-tert-butyl(4-dimethylaminophenyl)phosphine)dichloropalladium(II) (0.847 g). The flask was sealed, and the material was sparged for 60 minutes by blowing nitrogen over the material with stirring. Separately, in a 250 mL round bottom flask were added tetrahydrofuran (100 mL) and water (25 mL). The mixture was sparged sub-surface with stirring for 60 minutes by bubbling nitrogen through it. The sparged mixture was transferred via cannula to the flask with the material, and the reaction mixture was stirred overnight at room temperature. LC/MS indicated a single product had formed that corresponded to the desired product. The reaction mixture was diluted with ethyl acetate and water. Ammonium pyrrolidine dithiocarbamate (APDTC, 600 mgs, 3 equiv based on moles of Pd) was added as palladium scavenger, and mixture was stirred for 60 minutes. The mixture was poured into a separatory funnel, and the layers were separated. The organic layer was washed with brine, dried with Na₂SO₄, filtered, and concentrated. The residue was dissolved in dichloromethane and was purified on a Grace Reveleris X2 MPLC using a Teledyne Isco RediSep® Rf gold 330 g silica gel column eluting with 20-40% of (25% ethanol in ethyl acetate)/heptane. The desired product containing fractions were combined and concentrated to provide the title compound. ¹H NMR indicated atropisomers in an 8:1 ratio. Analytical HPLC of this material on a HP Agilent instrument using a Thermo Scientific HPLC column (Hypersil Gold AQ, 3.0 um, 150×4.6 mm) and a 30 minute gradient run from 10% to 90% acetonitrile in a trifluoroacetic acid buffer indicated the major atropisomer was 82% of the material with a retention time of 20.2 minutes and the minor atropisomer was 10% of the material with a retention time of 20.8 minutes. The crude material was carried on in the next step without further purification. MS (ESI) m/z 875.2 (M+H)⁺.

### Example 1U

### (R)-ethyl 2-((5-((1S)-3-chloro-4-formyl-2-methylphenyl)-6-(4-fluorophenyl)thieno[2,3-d]pyrimidin-4-yl)oxy)-3-(5-formyl-2-((2-(2-methoxyphenyl)pyrimidin-4-yl)methoxy)phenyl)propanoate

A 100 mL round bottom flask equipped with a stir bar was charged with Example 1T (2.98 g). The material was dissolved at room temperature in dichloromethane (6.81 mL). To the mixture was added trifluoroacetic acid (10 mL) and water (0.123 mL). The reaction mixture was stirred overnight at room temperature. Thin-layer chromatography in 20% ethyl acetate/dichloromethane indicated the reaction mixture was complete. The solvents were concentrated with a 50° C. bath and house vacuum. The material that resulted was dissolved in ethyl acetate and poured into water. The mixture was diluted further with ethyl acetate and water, and the layers were separated. The organic layer was washed with saturated aqueous NaHCO₃ mixture and brine, dried with Na₂SO₄, filtered, and concentrated. The residue was dissolved in dichloromethane and purified on a Grace Reveleris X2 MPLC using a Grace Reveleris 120 g silica gel column eluting with a 30 minute ramp of 10-30% ethyl acetate/dichloromethane. The desired product containing fractions were combined, and the solvents were concentrated to provide the title compound. ¹H NMR indicated an 8 to 1 mixture of atropisomers. Analytical HPLC of this material on a HP Agilent instrument using a Thermo Scientific HPLC column (Hypersil Gold AQ, 3.0 um, 150×4.6 mm) and a 30 minute gradient run from 10-90% acetonitrile in a trifluoroacetic acid buffer indicated the major atropisomer was 87% of the material with a retention time of 19.3 minutes and the minor atropisomer was 12% of the material with a retention time of 19.8 minutes. The crude material was carried on in the next step without further purification. MS (ESI) m/z 817.2 (M+H)⁺.

### Example 1V

### ethyl (7R,20S)-18-chloro-1-(4-fluorophenyl)-10-{[2-(2-methoxyphenyl)pyrimidin-4-yl]methoxy}-19-methyl-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylate

A 250 mL round bottom flask equipped with a stir bar was charged with Example 1U (1.96 g) and anhydrous dichloromethane (160 mL) at room temperature under nitrogen. The mixture was cooled to 0° C. in an ice bath, and 2-(4-methylpiperazin-1-yl)ethanamine (0.395 mL) was added via a syringe. The mixture was stirred for 25 minutes at 0° C., and sodium triacetoxyborohydride (156 mg) was added as a solid. The reaction mixture was stirred for 15 minutes at 0° C., and powdered activated 3 angstrom molecular sieves were added (1.96 g). The reaction mixture was stirred 2 hours at 0° C., and was allowed to stir and warm slowly to room temperature overnight. LC/MS indicated one major peak with a mass that corresponded to desired product. The reaction mixture was quenched with dichloromethane and water. The layers were separated, and aqueous layer was extracted with dichloromethane and 10% methanol/dichloromethane. The aqueous layer was neutralized with saturated aqueous NaHCO₃ mixture, and was extracted one more time with 10% methanol/dichloromethane. The combined extracts were washed with saturated aqueous NaHCO₃ and brine, dried with Na₂SO₄, filtered, and concentrated. The residue was dissolved in dichloromethane and was purified on a Grace Reveleris X2 MPLC using a Teledyne Isco RediSep® Rf gold 750 g silica gel column eluting with a gradient of 0-20% of methanol/dichloromethane over 40 minutes. The mixed fractions were purified on a Grace Reveleris X2 MPLC using a Teledyne Isco RediSep® Rf gold 330 g silica gel column eluting with a ramp of 0-15% of methanol/dichloromethane over 40 minutes to collect additional title compound. The material from both columns was combined to provide the title compound. ¹H NMR (501 MHz, dimethyl sulfoxide-d₆) δ ppm 8.61 (m, 2H), 7.47 (m, 2H), 7.39 (d, 1H), 7.17 (m, 7H), 7.04 (td, 1H), 6.96 (dd, 1H), 6.67 (d, 1H), 6.51 (d, 1H), 5.84 (dd, 1H), 5.06 (m, 2H), 4.07 (ddq, 2H), 3.90 (d, 1H), 3.75 (s, 3H), 3.68 (dd, 2H), 3.50 (d, 1H), 3.17 (m, 1H), 3.08 (m, 1H), 2.90 (m, 2H), 2.65-2.20 (m, 10H), 2.14 (s, 3H), 1.67 (s, 3H), 1.09 (t, 3H). MS (ESI) m/z 928.4 (M+H)⁺.

### Example 1W

### (7R,20S)-18-chloro-1-(4-fluorophenyl)-10-{[2-(2-methoxyphenyl)pyrimidin-4-yl]methoxy}-19-methyl-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylic acid

A 50 mL round bottom flask equipped with a stir bar was charged with Example 1V (1.07 g). The material was dissolved in tetrahydrofuran (5 mL). To the mixture at room temperature was added water (5.00 mL), solid LiOH (0.552 g), and methanol (1 mL). The mixture was stirred overnight at room temperature. LC/MS indicated the reaction mixture was about 60% complete. Another 500 mg of LiOH was added along with another 1 mL of methanol and 2 mL of water. After six more hours at room temperature, LC/MS indicated one major peak with a mass that corresponded to desired product. The reaction mixture was diluted with water, and ethyl acetate was added. The cloudy, two-phase mixture was stirred for 10 minutes. The layers were separated. The aqueous layer had a pH of about 9 and was neutralized to pH 7 with saturated aqueous NH₄Cl mixture. The aqueous phase was extracted with ethyl acetate. The combined organic extracts were washed with saturated aqueous NH₄Cl mixture and brine, dried with Na₂SO₄, filtered, and concentrated. The residue was dissolved in dichloromethane with about 2% methanol and purified on a Grace Reveleris X2 MPLC using a Teledyne Isco RediSep® Rf gold 40 g silica gel column eluting with a gradient over 20 minutes of 10-40% methanol/dichloromethane, and then a gradient over 10 minutes of 40-60% methanol/dichloromethane. Most of the desired product eluted during the second gradient. The desired product-containing fractions were combined, and the solvents were concentrated to provide the title compound. ¹H NMR (501 MHz, dimethyl sulfoxide-d₆) δ ppm 8.54 (m, 2H), 7.46 (m, 2H), 7.38 (d, 1H), 7.26 (d, 1H), 7.15 (m, 4H), 7.03 (m, 3H), 6.90 (dd, 1H), 6.59 (m, 2H), 5.87 (dd, 1H), 5.08 (d, 1H), 4.95 (d, 1H), 3.90-3.30 (m, 5H), 3.74 (s, 3H), 3.26 (dd, 1H), 3.03 (dd, 1H), 2.87 (m, 2H), 2.60-2.40 (m, 10H), 2.25 (s, 3H), 1.55 (s, 3H). MS (ESI) m/z 900.42 (M+H)⁺.

### Example 2

### (5R)-21-(4-fluorophenyl)-8-{[2-(2-methoxyphenyl)pyrimidin-4-yl]methoxy}-13-[2-(4-methylpiperazin-1-yl)ethyl]-5,6,13,14-tetrahydro-12H-15,20-etheno-11,7-(metheno)-4-oxa-22-thia-1,3,13-triazabenzo[16,17]cyclooctadeca[1,2,3-cd]indene-5-carboxylic acid

### Example 2A

### (R)-ethyl 2-((5-bromo-6-(4-fluorophenyl)thieno[2,3-d]pyrimidin-4-yl)oxy)-3-(5-(((tert-butoxycarbonyl)(2-(4-methylpiperazin-1-yl)ethyl)amino)methyl)-2-((2-(2-methoxyphenyl)pyrimidin-4-yl)methoxy)phenyl)propanoate

To a mixture of Example 1P (1.2 g) in dichoroethane (10 mL) was added 2-(4-methylpiperazin-1-yl)ethanamine (359 mg). The mixture was stirred at room temperature for 1 hour before the addition of sodium triacetoxyborohydride (800 mg). The mixture was stirred at room temperature for 3 hours and was quenched by the addition of saturated aqueous sodium bicarbonate mixture. The reaction mixture was extracted with ethyl acetate (200 mL×2). The combined organic extracts were washed with water and brine, and dried over sodium sulfate. Filtration and concentration of the filtrate provided a residue, which was dissolved in tetrahydrofuran (20 mL). Di-tert-butyldicarbonate (0.45 g) was added, followed by a catalytic amount of 4-N,N-dimethylaminopyridine. The mixture was stirred at room temperature for 2 hours. LC/MS showed the reaction was complete. The mixture was diluted with ethyl acetate (300 mL), washed with water and brine, and dried over sodium sulfate. Filtration and concentration of the filtrate provided a residue, which was purified by silica gel chromatography on a Grace Reveleris X2 MPLC and Grace Reveleris 80 g silica gel column, eluting with 5% 7N ammonium in methanol in dichloromethane to provide the title compound. MS (ESI) m/z 972.0 (M+H)⁺.

### Example 2B

### (2R)-ethyl 3-(5-(((tert-butoxycarbonyl)(2-(4-methylpiperazin-1-yl)ethyl)amino)methyl)-2-((2-(2-methoxyphenyl)pyrimidin-4-yl)methoxy)phenyl)-2-((6-(4-fluorophenyl)-5-(4-formylnaphthalen-1-yl)thieno[2,3-d]pyrimidin-4-yl)oxy)propanoate

(4-Formylnaphthalen-1-yl)boronic acid (24 mg), Example 2A (98 mg), bis(di-tert-butyl(4-dimethylaminophenyl)phosphine)dichloropalladium(II) (7.15 mg) and potassium carbonate (42 mg) were placed in 20 mL vial. Tetrahydrofuran (8 mL) and water (3 mL) were added, and the reaction mixture was purged with argon. The reaction mixture was stirred at room temperature over a weekend. The mixture was concentrated under vacuum. The residue was dissolved in ethyl acetate (300 mL), washed with water and brine, and dried over sodium sulfate. Filtration and concentration provided the title compound, which was used in the next reaction without further purification. MS (ESI) m/z 1046.43 (M+H)⁺.

### Example 2C

### (5R)-21-(4-fluorophenyl)-8-{[2-(2-methoxyphenyl)pyrimidin-4-yl]methoxy}-13-[2-(4-methylpiperazin-1-yl)ethyl]-5,6,13,14-tetrahydro-12H-15,20-etheno-11,7-(metheno)-4-oxa-22-thia-1,3,13-triazabenzo[16,17]cyclooctadeca[1,2,3-cd]indene-5-carboxylic acid

Example 2B (120 mg) was dissolved in dichloromethane and trifluoroacetic acid (10 mL, 1:1). The mixture was stirred at room temperature for 1 hour. LC/MS showed the deprotection was complete. The solvents were evaporated under vacuum, and the residue was dissolved in ethyl acetate (300 mL). The mixture was washed with saturated aqueous sodium bicarbonate mixture and brine, dried over sodium sulfate, and filtered. Concentration of the filtrate provided a residue, which was dissolved in dichloromethane (20 mL). Magnesium sulfate (anhydrous, 2.0 g) was added, and the mixture was stirred at room temperature for 1 hour before the addition of sodium triacetoxyborohydride (140 mg). The mixture was stirred for 1 hour. The mixture was partitioned between saturated aqueous sodium bicarbonate mixture (100 mL) and ethyl acetate (200 mL). The organic layer was washed with brine, dried over sodium sulfate, and filtered. Concentration of the filtrate provided a residue, which was dissolved in tetrahydrofuran/methanol/water (2:1:1, 10 mL). LiOH water (300 mg) was added. The mixture was stirred for 4 hours until LC/MS showed the saponification was complete. The mixture was concentrated under vacuum. The residue was dissolved in N,N-dimethylformamide (20 mL) and water (5 mL) and acidified with trifluoroacetic acid. The mixture was filtered and loaded on a Gilson HPLC (Phenomenex®, 250×50 mm, C-18 column). The column was eluted with 20 to 85% acetonitrile in water (0.1% trifluoroacetic acid) in 35 minutes to provide the title compound. ¹H NMR (501 MHz, dimethyl sulfoxide-d₆) δ ppm 8.74 (d, 1H), 8.69 (s, 1H), 8.01 (d, 1H), 7.80 (d, 1H), 7.55-7.43 (m, 5H), 7.38 (t, 1H), 7.24-7.13 (m, 4H), 7.05 (dt, 4H), 6.56 (d, 1H), 5.74 (s, 1H), 5.66 (dd, 1H), 5.06 (d, 1H), 4.97 (d, 1H), 4.90 (d, 1H), 4.25 (s, 2H), 3.76 (s, 3H), 3.10 (q, 3H), 2.81 (s, 3H), 2.50 (m, 10H). MS (ESI) m/z 902.2 (M+H)⁺.

### Example 3

### (7R,20S)-1-(4-fluorophenyl)-10-{[2-(2-methoxyphenyl)pyrimidin-4-yl]methoxy}-18,19-dimethyl-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylic acid

### Example 3A

### (2R)-ethyl 3-(5-(((tert-butoxycarbonyl)(2-(4-methylpiperazin-1-yl)ethyl)amino)methyl)-2-((2-(2-methoxyphenyl)pyrimidin-4-yl)methoxy)phenyl)-2-((6-(4-fluorophenyl)-5-(4-formyl-2,3-dimethylphenyl)thieno[2,3-d]pyrimidin-4-yl)oxy)propanoate

The title compound was prepared as described in Example 2B by replacing (4-formylnaphthalen-1-yl)boronic acid with (4-formyl-2,3-dimethylphenyl)boronic acid. MS (ESI) m/z 1024.32 (M+H)⁺.

### Example 3B

### (7R,20S)-1-(4-fluorophenyl)-10-{[2-(2-methoxyphenyl)pyrimidin-4-yl]methoxy}-18,19-dimethyl-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylic acid

The title compound was prepared as described in Example 2C, replacing Example 2B with Example 3A. ¹H NMR (400 MHz, dimethyl sulfoxide-d₆) δ ppm 8.77 (d, 1H), 8.68 (s, 1H), 7.54 (dd, 1H), 7.47 (ddd, 1H), 7.37 (d, 2H), 7.28 (ddd, 3H), 7.15 (td, 3H), 7.11-7.01 (m, 2H), 6.95 (d, 1H), 6.15 (d, 1H), 5.96 (dd, 1H), 5.32-5.14 (m, 2H), 4.24 (d, 2H), 3.77 (s, 3H), 3.71-2.91 (m, 5H), 2.79 (s, 3H), 1.89 (s, 3H), 1.85 (s, 3H). MS (ESI) m/z 880.2 (M+H)⁺.

### Example 4

### (7R,20S)-1-(4-fluorophenyl)-10-{[2-(2-methoxyphenyl)pyrimidin-4-yl]methoxy}-19-methyl-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylic acid

### Example 4A

### (2R)-ethyl 3-(5-(((tert-butoxycarbonyl)(2-(4-methylpiperazin-1-yl)ethyl)amino)methyl)-2-((2-(2-methoxyphenyl)pyrimidin-4-yl)methoxy)phenyl)-2-((6-(4-fluorophenyl)-5-(4-formyl-2-methylphenyl)thieno[2,3-d]pyrimidin-4-yl)oxy)propanoate

The title compound was prepared as described in Example 2B by replacing (4-formylnaphthalen-1-yl)boronic acid with (4-formyl-2-methylphenyl)boronic acid. MS (ESI) m/z 1010.22 (M+H)⁺.

### Example 4B

### (7R,20S)-1-(4-fluorophenyl)-10-{[2-(2-methoxyphenyl)pyrimidin-4-yl]methoxy}-19-methyl-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylic acid

The title compound was prepared as described in Example 2C by replacing Example 2B with Example 4A. ¹H NMR (501 MHz, dimethyl sulfoxide-d₆) δ ppm 8.71 (d, 1H), 8.61 (d, 1H), 8.52 (d, 1H), 7.58-7.43 (m, 3H), 7.38-7.25 (m, 4H), 7.23-7.08 (m, 7H), 7.05-6.98 (m, 2H), 6.71 (s, 1H), 6.62-6.56 (m, 1H), 5.93 (dd, 1H), 5.25-5.07 (m, 3H), 4.62-4.26 (m, 5H), 3.74 (d, 13H), 3.69-2.97 (m, 18H), 2.80 (s, 4H), 2.34 (s, 1H), 1.57 (s, 3H). MS (ESI) m/z 866.2 (M+H)⁺.

### Example 5

### (7R,20S)-18,19-difluoro-1-(4-fluorophenyl)-10-{[2-(2-methoxyphenyl)pyrimidin-4-yl]methoxy}-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylic acid

### Example 5A

### (2R)-ethyl 3-(5-(((tert-butoxycarbonyl)(2-(4-methylpiperazin-1-yl)ethyl)amino)methyl)-2-((2-(2-methoxyphenyl)pyrimidin-4-yl)methoxy)phenyl)-2-((5-(2,3-difluoro-4-formylphenyl)-6-(4-fluorophenyl)thieno[2,3-d]pyrimidin-4-yl)oxy)propanoate

The title compound was prepared as described in Example 2B by replacing (4-formylnaphthalen-1-yl)boronic acid with (2,3-difluoro-4-formylphenyl)boronic acid. MS (ESI) m/z 1032.33 (M+H)⁺.

### Example 5B

### (7R,20S)-18,19-difluoro-1-(4-fluorophenyl)-10-{[2-(2-methoxyphenyl)pyrimidin-4-yl]methoxy}-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylic acid

The title compound was prepared as described in Example 2C by replacing Example 2B with Example 5A. ¹H NMR (500 MHz, dimethyl sulfoxide-d₆) δ ppm 8.62 (s, 1H), 8.52 (d, 1H), 7.51-7.41 (m, 2H), 7.29-7.23 (m, 2H), 7.22-7.12 (m, 3H), 7.08 (d, 1H), 7.03 (td, 2H), 6.85 (d, 1H), 6.78 (d, 1H), 6.67 (t, 1H), 6.41-6.31 (m, 1H), 5.97 (dd, 1H), 5.22-5.06 (m, 2H), 4.41 (d, 1H), 4.09-3.82 (m, 7H), 3.73 (s, 3H), 3.50 (dd, 1H), 3.18 (d, 5H), 2.81 (s, 3H). MS (ESI) m/z 888.1 (M+H)⁺.

### Example 6

### (7R,20S)-19-chloro-1-(4-fluorophenyl)-10-{[2-(2-methoxyphenyl)pyrimidin-4-yl]methoxy}-18-methyl-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylic acid

### Example 6A

### (2R)-ethyl 3-(5-(((tert-butoxycarbonyl)(2-(4-methylpiperazin-1-yl)ethyl)amino)methyl)-2-((2-(2-methoxyphenyl)pyrimidin-4-yl)methoxy)phenyl)-2-((5-(2-chloro-4-formyl-3-methylphenyl)-6-(4-fluorophenyl)thieno[2,3-d]pyrimidin-4-yl)oxy)propanoate

The title compound was prepared as described in Example 2B by replacing (4-formylnaphthalen-1-yl)boronic acid with (2-chloro-4-formyl-3-methylphenyl)boronic acid. MS (ESI) m/z 1044.72 M+H)⁺.

### Example 6B

### (7R,20S)-19-chloro-1-(4-fluorophenyl)-10-{[2-(2-methoxyphenyl)pyrimidin-4-yl]methoxy}-18-methyl-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylic acid

The title compound was prepared as described in Example 2C by replacing Example 2B with Example 6A. ¹H NMR (501 MHz, dimethyl sulfoxide-d₆) δ ppm 8.62-8.56 (m, 2H), 7.53-7.40 (m, 2H), 7.28-7.21 (m, 3H), 7.19-7.10 (m, 3H), 7.08-6.94 (m, 2H), 6.80 (t, 2H), 6.55-6.40 (m, 2H), 5.83 (dd, 1H), 5.15 (s, 2H), 4.42 (d, 1H), 3.95 (d, 2H), 3.74 (s, 3H), 3.46 (dd, 1H), 3.39-2.91 (m, 4H), 2.79 (s, 3H), 2.67 (s, 3H). MS (ESI) m/z 900.2 (M+H)⁺.

### Example 7

### (7R,20S)-18-chloro-1-(4-fluorophenyl)-19-methyl-15-oxo-16-[2-(piperazin-1-yl)ethyl]-10-{[2-(3,3,3-trifluoropropoxy)pyrimidin-4-yl]methoxy}-7,8,15,16-tetrahydro-14H-17,20-etheno-13,9-(metheno)-6-oxa-2-thia-3,5,16-triazacyclooctadeca[1,2,3-cd]indene-7-carboxylic acid

### Example 7A

### 4-(dimethoxymethyl)-2-(methylthio)pyrimidine

A dried 1 L three-neck round bottom flask equipped with a stir bar and an internal temperature probe (J-KEM controlled) was charged with solid sodium methoxide (24.95 g) under nitrogen at room temperature. The flask was cooled in a NaCl-ice water bath as anhydrous methanol (257 mL) was added. The internal temperature monitored by J-KEM indicated a temperature rise of about 7° C. upon addition of the methanol. The colorless slurry that resulted was cooled to about 3.6° C. To the mixture was added portionwise thiourea (26.4 g) over the course of about 5 minutes. The addition was slightly endothermic with the temperature dropping to 2.4° C. The reaction mixture was stirred for 60 minutes at about 1.0° C. To the mixture at 1.6° C. was added (E)-4-(dimethylamino)-1,1-dimethoxybut-3-en-2-one (40 g) dropwise via an addition funnel. The addition took about 10 minutes, and a slight temperature rise from 1.6° C. to 3.6° C. was observed. The cooling bath was removed, and the reaction mixture was heated to about 65° C. After three hours of heating, thin-layer chromatography in 5% methanol/dichloromethane indicated the reaction mixture was nearly complete. The reaction mixture was heated an additional hour. The heating block was removed, and the reaction was cooled in an ice bath to about 3.5° C. Iodomethane (19.49 mL) was added dropwise via an addition funnel. The temperature rose to 9.4° C., and the addition took about 10 minutes. The mixture was stirred overnight at room temperature. The reaction mixture was filtered, and the collected material was washed with additional methanol. The solvents were concentrated, and the residue was dissolved in ethyl acetate. The organic layer was washed with water (twice) and brine. The combined aqueous layers were back extracted with diethyl ether. The combined extracts were dried with Na₂SO₄, filtered, and concentrated. The residue was mixed in 1:1 dichloromethane/heptane and poured onto the top of a pad of silica (about 1.4 L silica) that had been equilibrated in a 3 L fritted Buchner funnel with 10% ethyl acetate/heptane. The title compound was eluted with 10% to 20% to 30% ethyl acetate in heptane. The pure fractions of title compound were collected and concentrated to provide the title compound. ¹H NMR (400 MHz, dimethyl sulfoxide-d₆) δ ppm 8.66 (d, 1H), 7.21 (d, 1H), 5.20 (s, 1H), 3.31 (s, 6H), 2.50 (s, 3H). MS (DCI) m/z 200.9 (M+H)⁺.

### Example 7B

### (2-(methylthio)pyrimidin-4-yl)methanol

A 2 L flask fitted with an internal temperature probe (J-KEM controlled) and stir bar was charged with Example 7A (17.4 g). To the mixture was added at room temperature 2N aqueous HCl mixture (261 mL). The addition was slightly exothermic. The mixture was heated to 60° C. for three hours. Heating was stopped, and as the reaction mixture was cooled to 37° C., 1,4-dioxane (260 mL) was added. The mixture was cooled to −9.7° C. in an ice/methanol bath. Powdered NaOH (19.11 g) was added in portions over about one hour. The temperature rose to about 1.3° C. during the addition. The reaction mixture was stirred until all the solid NaOH was dissolved (pH was about 2 at this point). NaOH mixture (1N aqueous) was added in 10 mL portions until the pH was about 8 by pH paper. The temperature rose to 4.3° C. during the addition. The reaction mixture was allowed to cool to −0.9° C., and solid NaBH₄ (6.57 g) was added to the mixture in portions over about 5 minutes, during which the temperature of the reaction went up to 4.5° C. The reaction mixture was allowed to stir in the cold bath for 1 hour. To the reaction mixture was added 100 mL of 30% methanol/dichloromethane. The two-phase mixture was stirred for about 15 minutes. The layers were separated, and aqueous layer was extracted once with 100 mL of 30% methanol/dichloromethane. Thin-layer chromatography of the aqueous layer still indicated desired product remained. Another 100 mL of 30% methanol/dichloromethane was added to the aqueous layer, and two-phase mixture was stirred overnight. The layers were separated, and aqueous layer was extracted once with 100 mL of 30% methanol/dichloromethane. Thin-layer chromatography of the aqueous layer still indicated some desired product. Brine was added to the aqueous layer, and 100 mL of 40% methanol/dichloromethane was added. The two-phase mixture was stirred for two hours. The layers were separated, and the combined organic extracts were dried with Na₂SO₄, filtered, and concentrated. The crude material was pre-absorbed on 50 g of silica gel and purified on a Grace Reveleris X2 MPLC using a Teledyne Isco RediSep® Rf gold 220 g silica gel column, eluting with a 0% to 40% gradient over 30 minutes of ethyl acetate/dichloromethane. The pure fractions were combined and concentrated to provide the title compound. ¹H NMR (400 MHz, dimethyl sulfoxide-d₆) δ ppm 8.61 (d, 1H), 7.25 (dt, 1H), 5.63 (t, 1H), 4.50 (m, 2H), 2.50 (s, 3H). MS (DCI) m/z 156.9 (M+H)⁺.

### Example 7C

### 4-(dimethoxymethyl)-2-(methylsulfonyl)pyrimidine

Example 7B (117 g) was dissolved in 1 L methanol and charged into a 5 L fully-jacketed round-bottom flask connected to a Huber 230 circulator and fit with overhead stirring and a thermocouple. Water (1 L) was added, and the temperature was set to 0° C. When the reaction temperature reached about 2.0° C., Oxone® (potassium peroxymonosulfate, 467 g) was added portionwise over about 20 minutes, noting a slight and easily controlled rise in temperature (2-3° C., reaction). The slurry was stirred overnight at 0° C. The reactor temperature was increased to 20° C., and the methanol was removed (bulb to bulb) under vacuum, increasing the flask temperature to 40° C., collecting about 750 mL methanol in a dry ice/acetone cooled receiving flask. The remaining slurry was filtered through paper. The material was washed twice with dichloromethane, and the biphasic filtrate was separated. The aqueous layer was extracted twice with dichloromethane. The combined organics were dried (MgSO₄), filtered and concentrated by rotary evaporation to provide the title compound. ¹H NMR (501 MHz, dimethyl sulfoxide-d₆) δ ppm 9.16 (d, 1H), 7.88 (d, 1H), 5.46 (s, 1H), 3.45 (s, 3H), 3.40 (s, 6H). MS (ESI) m/z 250.0 (M+NH₄)⁺.

### Example 7D

### 4-(dimethoxymethyl)-2-(3,3,3-trifluoropropoxy)pyrimidine

Example 7C (128 g), potassium carbonate (152 g) and acetonitrile (1837 mL) were combined in a 5 L round bottom flask equipped with mechanical stirring, JKEM/thermocouple, reflux condenser and a light nitrogen flow. 3,3,3-Trifluoropropan-1-ol (35.5 mL) was added neat, and the reaction mixture was heated to 58° C. overnight. An additional 40 g of 3,3,3-trifluoropropan-1-ol was added and the mixture was heated at 80° C. again overnight. Thin-layer chromatography indicated a single spot (1:1 ethyl acetate:heptanes) with just a little starting material remaining. The reaction mixture was cooled to room temperature and was filtered. The filtrate was treated with charcoal, stirred for 60 minutes, filtered through a plug of diatomaceous earth, and concentrated by rotary evaporation. The residue was passed through a silica gel plug (1.5 L silica gel), using ethyl acetate:heptanes (1:1) to elute. The collected fractions were concentrated by rotary evaporation to provide the title compound. ¹H NMR (400 MHz, dimethyl sulfoxide-d₆) δ ppm 8.68 (d, 1H), 7.23 (d, 1H), 5.23 (s, 1H), 4.55 (t, 2H), 3.34 (d, 6H), 2.98-2.73 (m, 2H). MS (DCI) m/z 267.0 (M+H)⁺.

### Example 7E

### (2-(3,3,3-trifluoropropoxy)pyrimidin-4-yl)methanol

Example 7D (137 g, 515 mmol) and acetonitrile (1.715 L) were combined in a 5 L round-bottom flask. Aqueous HCl (2 N, 1 L) was added, and the mixture was stirred at 60° C. for 1 hour. The reaction mixture was cooled in an ice bath, achieving an internal temperature of about 5° C., and 2 N aqueous NaOH (0.901 L) was added followed by solid K₂CO₃ until the pH was ˜ 8. Sodium borohydride was added portionwise. After 1 hour, a single peak by LC/MS indicated product formation. Ethyl acetate (1 L) was added, and the layers were separated. The aqueous layer was extracted with ethyl acetate (three times). Charcoal and MgSO₄ were added to the combined organic layers and the mixture was stirred overnight. The mixture was filtered through a short plug of silica to remove much of the color. The filtrate was concentrated to give coarse material, which were milled and bottled to provide the title compound. ¹H NMR (400 MHz, chloroform-d) δ ppm 8.45 (d, 1H), 7.05 (dd, 1H), 4.69 (d, 2H), 4.58 (t, 2H), 3.67 (t, 1H), 2.76-2.51 (m, 2H). MS (DCI) m/z 223.0 (M+H)⁺.

### Example 7F

### (R)-ethyl 2-acetoxy-3-(5-bromo-2-((2-(3,3,3-trifluoropropoxy)pyrimidin-4-yl)methoxy)phenyl)propanoate

Example 7F was made according to the procedure described for Example 1L, substituting Example 7E for Example 1G. ¹H NMR (400 MHz, dimethyl sulfoxide-d₆) δ ppm 8.68 (d, 1H), 7.52-7.36 (m, 2H), 7.29 (d, 1H), 7.01 (d, 1H), 5.25-5.10 (m, 3H), 4.54 (t, 2H), 4.07 (q, 2H), 3.26 (dd, 1H), 3.11 (dd, 1H), 2.93-2.72 (m, 2H), 2.02 (s, 3H), 1.10 (t, 3H). MS (ESI−) m/z 534.9 (M+H)⁺.

### Example 7G

### 4-bromo-2-chloro-3-methylaniline

To a mixture of 2-chloro-3-methylaniline (1.83 g) and ammonium acetate (100 mg) in acetonitrile (64.6 mL), was added N-bromosuccinimide (2.42 g), and the mixture was stirred at room temperature. After completion of the reaction as indicated by thin-layer chromatography, the mixture was concentrated onto silica gel. Purification by flash chromatography on a CombiFlash® Teledyne Isco system using a Teledyne Isco RediSep® Rf gold 80 g silica gel column (eluting with 0-30% ethyl acetate/heptane) provided the title compound. LC/MS (APCI) m/z 222.3 (M+H)⁺.

### Example 7H

### 2-chloro-3-methyl-4-(4,4,5,5-tetramethyl-1,3,2-dioxaborolan-2-yl)aniline

To a 25 mL flask was added potassium acetate (2.44 g), and the vessel was capped with septum and heated to 100° C. under high vacuum for 1 hour. After cooling to ambient temperature, bis(pinacolato)diboron (4.22 g), Example 7G (1.83 g), 2-(dicyclohexylphosphino)-2′,4′,6′-triisopropylbiphenyl (0.119 g) and chloro(2-dicyclohexylphosphino-2′,4′,6′-triisopropyl-1,1′-biphenyl)[2-(2′-amino-1,1′-biphenyl)]palladium(II) (0.196 g) were quickly added. The vessel was capped again, evacuated and backfilled with nitrogen three times. Freshly degassed 2-methyltetrahydrofuran (83 mL; nitrogen was bubbled through the solvent for 30 minutes prior addition) was introduced via syringe. The stirring mixture was evacuated and backfilled with nitrogen twice again. The mixture was stirred at 75° C. for 6 hours and cooled to ambient temperature. The mixture was filtered through a bed of diatomaceous earth, eluted with 20 mL of ethyl acetate, and concentrated onto silica gel. Purification by silica gel chromatography on a CombiFlash® Teledyne Isco system using a Teledyne Isco RediSep® Rf gold 24 g silica gel column (eluting with 0-30% ethyl acetate/heptane) provided the title compound. LC/MS (APCI) m/z 268.2 (M+H)⁺.

### Example 7I

### (R)-ethyl 2-acetoxy-3-(5-allyl-2-((2-(3,3,3-trifluoropropoxy)pyrimidin-4-yl)methoxy)phenyl)propanoate

A round bottom flask equipped with a stir bar and a reflux condenser was charged with Example 7F (2 g), 1,1′-bis(diphenylphosphino)ferrocene-palladium(II)dichloride dichloromethane complex (0.458 g) and cesium fluoride (2.55 g). The flask was capped with a septum and sparged with nitrogen. Degassed anhydrous tetrahydrofuran was added followed by 2-allyl-4,4,5,5-tetramethyl-1,3,2-dioxaborolane (1.57 g). The mixture was evacuated and backfilled with nitrogen twice, stirred at 75° C. for 4 hours, and cooled back to ambient temperature. The resulting mixture was filtered through a one inch thick diatomaceous earth pad, and the filter cake was washed with 200 mL of ethyl acetate. The filtrate was concentrated onto silica gel and purification by silica gel flash chromatography on a CombiFlash® Teledyne Isco system using a Teledyne Isco RediSep® Rf gold 120 g silica gel column (eluting with 10-100% ethyl acetate/heptane) provided the title compound. LC/MS (APCI) m/z 497.2 (M+H)⁺.

### Example 7J

### (R)-2-(3-(2-acetoxy-3-ethoxy-3-oxopropyl)-4-((2-(3,3,3-trifluoropropoxy)pyrimidin-4-yl)methoxy)phenyl)acetic acid

To a mixture of Example 71 (1.51 g) in carbon tetrachloride (18.1 mL) and acetonitrile (18.1 mL) at room temperature was added ruthenium(III) chloride trihydrate (0.119 g) and sodium periodate (3.25 g) as a mixture in water (27.2 mL). The mixture was stirred vigorously at ambient temperature for 90 minutes. The mixture was diluted with 50 mL of water, poured into a separatory funnel and extracted with three 50 mL portions of dichloromethane. The combined organic layers were dried over anhydrous magnesium sulfate, filtered and concentrated onto silica gel. Purification by silica gel chromatography on a CombiFlash® Teledyne Isco system using a Teledyne Isco RediSep® Rf gold 120 g silica gel column (eluting with solvent A=2:1 ethyl acetate:ethanol and solvent B=heptane; 10-100% A to B) provided the title compound. LC/MS (APCI) m/z 515.2 (M+H)⁺.

### Example 7K

### (R)-ethyl 2-acetoxy-3-(5-(2-(tert-butoxy)-2-oxoethyl)-2-((2-(3,3,3-trifluoropropoxy)pyrimidin-4-yl)methoxy)phenyl)propanoate

Example 7J (500 mg) was added to a 25 mL microwavable vessel and was treated with 3 mL of tert-butyl acetoacetate. Sulfuric acid (10 μL of) was added. The flask was capped, and the mixture was stirred at 40° C. for 48 hours. After cooling to −10° C., the cap was removed, and the mixture was concentrated, re-dissolved into dichloromethane, and concentrated onto silica gel. Purification by silica gel chromatography on a CombiFlash® Teledyne Isco system using a Teledyne Isco RediSep® Rf gold 24 g silica gel column (eluting with 10-100% ethyl acetate/heptane) provided the title compound. LC/MS (APCI) m/z 571.2 (M+H)⁺.

### Example 7L

### (R)-ethyl 3-(5-(2-(tert-butoxy)-2-oxoethyl)-2-((2-(3,3,3-trifluoropropoxy)pyrimidin-4-yl)methoxy)phenyl)-2-hydroxypropanoate

To a mixture of Example 7K (0.2 g) in ethanol (2.29 mL) was added anhydrous potassium carbonate (0.194 g), and the mixture was stirred at room temperature for 3 hours. The reaction mixture was poured into a separatory funnel containing water (30 mL) and was extracted with three portions of dichloromethane. The combined organic layers was dried over anhydrous magnesium sulfate, filtered and concentrated onto silica gel. Purification by silica gel chromatography on a CombiFlash® Teledyne Isco system using a Teledyne Isco RediSep® Rf gold 24 g silica gel column (eluting with 0-70% ethyl acetate/heptane) provided the title compound. LC/MS (APCI) m/z 529.3 (M+H)⁺.

### Example 7M

### (R)-ethyl 2-((5-bromo-6-(4-fluorophenyl)thieno[2,3-d]pyrimidin-4-yl)oxy)-3-(5-(2-(tert-butoxy)-2-oxoethyl)-2-((2-(3,3,3-trifluoropropoxy)pyrimidin-4-yl)methoxy)phenyl)propanoate

To a 50 mL round bottom flask containing Example 7L (135 mg) was added Example 1D (114 mg), cesium carbonate (283 mg) and tert-butanol (2.5 mL). The vial was capped, and the mixture was stirred at 65° C. for 2 hours. After cooling to ambient temperature, the mixture was concentrated to remove most of the tert-butanol. The residue was re-dissolved in ethyl acetate (25 mL) and poured into a separatory funnel. The resulting mixture was washed with water and saturated aqueous brine, dried over anhydrous magnesium sulfate, filtered and concentrated onto silica gel. Purification by silica gel chromatography on a CombiFlash® Teledyne Isco system using a Teledyne Isco RediSep® Rf gold 12 g silica gel column (eluting with 0-50% ethyl acetate/heptane) provided the title compound. LC/MS (APCI) m/z 835.1 (M+H)⁺.

### Example 7N

### (R)-ethyl 2-((5-((1S)-4-amino-3-chloro-2-methylphenyl)-6-(4-fluorophenyl)thieno[2,3-d]pyrimidin-4-yl)oxy)-3-(5-(2-(tert-butoxy)-2-oxoethyl)-2-((2-(3,3,3-trifluoropropoxy)pyrimidin-4-yl)methoxy)phenyl)propanoate

A 20 mL microwavable vessel, equipped with stir bar and septa, was charged with Example 7M (50 mg), Example 7H (20.8 mg), bis(di-tert-butyl(4-dimethylaminophenyl)phosphine)dichloropalladium(II) (4.24 mg) and cesium carbonate (58.5 mg). The vessel was capped and evacuated and backfilled with nitrogen twice. Freshly degassed tetrahydrofuran (0.6 mL) followed by water (0.15 mL) were introduced, and the reaction mixture was evacuated and backfilled with nitrogen twice again while stirring. The mixture was stirred at ambient temperature overnight. The mixture was poured into a separatory funnel, and diluted with ethyl acetate. The organic layer was washed with water and brine, dried over anhydrous magnesium sulfate, filtered and concentrated onto silica gel. Purification by silica gel chromatography on a CombiFlash® Teledyne Isco system using a Teledyne Isco RediSep® Rf gold 12 g silica gel column (eluting with 10-80% ethyl acetate/heptane) provided the title compound. LC/MS (APCI) m/z 896.2 (M+H)⁺.

### Example 7O

### 2-(3-((R)-2-((5-((1S)-4-amino-3-chloro-2-methylphenyl)-6-(4-fluorophenyl)thieno[2,3-d]pyrimidin-4-yl)oxy)-3-ethoxy-3-oxopropyl)-4-((2-(3,3,3-trifluoropropoxy)pyrimidin-4-yl)methoxy)phenyl)acetic acid

Example 7N (17.5 mg) was dissolved in 0.5 mL of dichloromethane and 0.5 mL of trifluoroacetic was added. The reaction mixture was stirred at ambient temperature for 75 minutes and concentrated to provide the title compound, which was used in the next step without further purification. LC/MS (APCI) m/z 839.9 (M+H)⁺.

### Example 7P

### ethyl (7R,20S)-18-chloro-1-(4-fluorophenyl)-19-methyl-15-oxo-10-{[2-(3,3,3-trifluoropropoxy)pyrimidin-4-yl]methoxy}-7,8,15,16-tetrahydro-14H-17,20-etheno-13,9-(metheno)-6-oxa-2-thia-3,5,16-triazacyclooctadeca[1,2,3-cd]indene-7-carboxylate

Example 7O (16.8 mg) was dissolved in dichloromethane (2 mL) and 1-[bis(dimethylamino)methylene]-1H-1,2,3-triazolo[4,5-b]pyridinium 3-oxid hexafluorophosphate (11.4 mg, HATU), 1-hydroxybenzotriazole hydrate (2.3 mg, HOBT), 4-dimethylaminopyridine (0.2 mg) and N,N-diisopropylethylamine (21 μL) were added successively. The reaction mixture was stirred at room temperature overnight. The mixture was concentrated, and the residue was dissolved in a small amount of dichloromethane and loaded on a 0.5 mm thick 20×20 cm preparative thin-layer chromatography plate (eluting with 75% ethyl acetate/heptane) to provide the title compound. LC/MS (APCI) m/z 822.1 (M+H)⁺.

### Example 7Q

### ethyl (7R,20S)-16-{2-[4-(tert-butoxycarbonyl)piperazin-1-yl]ethyl}-18-chloro-1-(4-fluorophenyl)-19-methyl-15-oxo-10-{[2-(3,3,3-trifluoropropoxy)pyrimidin-4-yl]methoxy}-7,8,15,16-tetrahydro-14H-17,20-etheno-13,9-(metheno)-6-oxa-2-thia-3,5,16-triazacyclooctadeca[1,2,3-cd]indene-7-carboxylate

A 4 mL vial equipped with stir bar and septum was charged with Example 7P (9.5 mg), tert-butyl 4-(2-bromomethyl)piperazine-1-carboxylate (6.8 mg) and cesium carbonate (11.3 mg). N,N-dimethylformamide (116 μL) was added, and the mixture was stirred at ambient temperature. After completion of the reaction as indicated by LC/MS (˜30 minutes), the mixture was poured into water and extracted with three portions of ethyl acetate. The combined organic layers were washed with brine, dried over anhydrous magnesium sulfate, filtered and concentrated. Purification by preparative thin-layer chromatography (0.5 mm thick, 20×20 cm, eluting with 100% ethyl acetate) provided the title compound. LC/MS (APCI) m/z 1034.4 (M+H)⁺.

### Example 7R

### (7R,20S)-18-chloro-1-(4-fluorophenyl)-19-methyl-15-oxo-16-[2-(piperazin-1-yl)ethyl]-10-{[2-(3,3,3-trifluoropropoxy)pyrimidin-4-yl]methoxy}-7,8,15,16-tetrahydro-14H-17,20-etheno-13,9-(metheno)-6-oxa-2-thia-3,5,16-triazacyclooctadeca[1,2,3-cd]indene-7-carboxylic acid

Example 7Q (11 mg) was dissolved in 0.5 mL of dichloromethane and was treated with 0.5 mL of trifluoroacetic acid. The mixture was stirred at ambient temperature for 10 minutes and was concentrated. The crude residue was dissolved in 0.3 mL of tetrahydrofuran and 0.3 mL of aqueous LiOH (1 molar) was added. The mixture was stirred at ambient temperature overnight. The volatiles were removed, and the aqueous mixture was acidified with few drops of trifluoroacetic acid. Acetonitrile was added to the mixture to solubilize the material, and the resulting mixture was purified directly on a Gilson reverse-phase prep LC (Zorbax, C-18, 250×2.54 column, Mobile phase A: 0.1% trifluoroacetic acid in water; B: 0.1% trifluoroacetic acid in acetonitrile; 10-100% B to A gradient) to provide the title compound. ¹H NMR (500 MHz, dimethyl sulfoxide-d₆) δ ppm 2.15 (s, 3H), 2.70-2.90 (m, 3H), 2.92-3.21 (m, 7H), 3.33 (q, 2H), 3.70 (dd, 1H), 4.06 (s, 4H), 4.30-4.38 (m, 1H), 4.53 (t, 2H), 5.12-5.24 (m, 2H), 5.94 (d, 1H), 6.42 (t, 1H), 6.91 (d, 1H), 7.06 (dd, 1H), 7.13 (d, 1H), 7.15-7.24 (m, 3H), 7.25-7.33 (m, 2H), 7.46 (d, 1H), 8.61 (d, 1H), 8.78 (s, 1H), 8.85 (s, 2H). LC/MS (APCI) m/z 906.2 (M+H)⁺.

### Example 8

### (7R,20S)-18-fluoro-1-(4-fluorophenyl)-19-methoxy-10-{[2-(2-methoxyphenyl)pyrimidin-4-yl]methoxy}-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylic acid

### Example 8A

### (2R)-ethyl 3-(5-(((tert-butoxycarbonyl)(2-(4-methylpiperazin-1-yl)ethyl)amino)methyl)-2-((2-(2-methoxyphenyl)pyrimidin-4-yl)methoxy)phenyl)-2-((5-(3-fluoro-4-formyl-2-methoxyphenyl)-6-(4-fluorophenyl)thieno[2,3-d]pyrimidin-4-yl)oxy)propanoate

The title compound was prepared as described in Example 2B by replacing (4-formylnaphthalen-1-yl)boronic acid with 2-fluoro-3-methoxy-4-(4,4,5,5-tetramethyl-1,3,2-dioxaborolan-2-yl)benzaldehyde. MS (ESI) m/z 1044.33 (M+H)⁺.

### Example 8B

### (7R,20S)-18-fluoro-1-(4-fluorophenyl)-19-methoxy-10-{[2-(2-methoxyphenyl)pyrimidin-4-yl]methoxy}-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylic acid

The title compound was prepared as described in Example 2C by replacing Example 2B with Example 8A. ¹H NMR (400 MHz, dimethyl sulfoxide-d₆) δ ppm 8.67-8.59 (m, 2H), 8.52 (d, 1H), 7.54-7.41 (m, 3H), 7.29-7.12 (m, 11H), 7.06-7.00 (m, 1H), 6.93-6.78 (m, 3H), 6.46 (t, 1H), 6.28 (d, 1H), 5.96 (ddd, 2H), 5.19 (s, 2H), 4.57 (d, 1H), 4.35-4.01 (m, 8H), 3.94 (d, J=2.1 Hz, 3H), 3.82-3.41 (m, 22H), 3.10 (s, 3H), 2.81 (s, 3H). MS (ESI) m/z 900.3 (M+H)⁺.

### Example 9

### (7R,20R)-18-chloro-1-(4-fluorophenyl)-19-methyl-16-[2-(4-methylpiperazin-1-yl)ethyl]-15-oxo-10-{[2-(3,3,3-trifluoropropoxy)pyrimidin-4-yl]methoxy}-7,8,15,16-tetrahydro-14H-17,20-etheno-13,9-(metheno)-6-oxa-2-thia-3,5,16-triazacyclooctadeca[1,2,3-cd]indene-7-carboxylic acid

Example 7Q (36 mg) was dissolved in 0.5 mL of dichloromethane and treated with 0.5 mL of trifluoroacetic acid. The mixture was stirred at ambient temperature for 10 minutes and was concentrated. The residue was dissolved in tetrahydrofuran (696 μL), and ˜37% aqueous mixture of formaldehyde (10 μL) followed by sodium triacetoxyborohydride (22.1 mg) were added. The resulting mixture was stirred at ambient temperature until completion of the reaction as indicated by LC/MS (˜30 minutes). Aqueous lithium hydroxide (1M, 696 μL) followed by 0.2 mL of methanol were added, and the mixture was stirred at ambient temperature overnight. The volatiles were removed, and the resulting aqueous mixture was acidified by dropwise addition of trifluoroacetic acid. Acetonitrile (1 mL) was added to dissolve the material, and the mixture was purified directly on a Gilson reverse-phase HPLC (Zorbax, C-18, 250×2.54 mm column, Mobile phase A: 0.1% trifluoroacetic acid in water; B: 0.1% trifluoroacetic acid in acetonitrile; 10-100% B to A gradient) to provide the title compound. ¹H NMR (501 MHz, dimethyl sulfoxide-d₆) δ ppm 2.13 (s, 3H), 2.57-2.72 (m, 4H), 2.74 (s, 3H), 2.76-2.86 (m, 2H), 2.98-3.11 (m, 2H), 3.12-3.25 (m, 4H), 3.30 (q, 2H), 3.69 (dd, 1H), 4.30 (dt, 1H), 4.51 (t, 2H), 5.10-5.21 (m, 2H), 5.93 (d, 1H), 6.41 (t, 1H), 6.90 (d, 1H), 7.04 (dd, 1H), 7.10 (d, 1H), 7.13-7.23 (m, 4H), 7.24-7.32 (m, 2H), 7.40 (d, 1H), 8.59 (d, 1H), 8.76 (s, 1H). LC/MS m/z (APCI) m/z 920.2 (M+H)⁺.

### Example 10

### (7R,21S)-19-chloro-1-(4-fluorophenyl)-20-methyl-16-[2-(4-methylpiperazin-1-yl)ethyl]-15-oxo-10-{[2-(3,3,3-trifluoropropoxy)pyrimidin-4-yl]methoxy}-7,8,14,15,16,17-hexahydro-18,21-etheno-13,9-(metheno)-6-oxa-2-thia-3,5,16-triazacyclononadeca[1,2,3-cd]indene-7-carboxylic acid

### Example 10A

### 4-bromo-2-chloro-3-methylbenzaldehyde

To a mixture of Example 1R (4.5 g) in tetrahydrofuran (27.0 mL) was slowly added 50 mL of 1 molar aqueous HCl mixture, and the mixture was refluxed for 4 hours. After cooling to ambient temperature, the mixture was diluted with ethyl acetate and water and partitioned between the two phases. The aqueous layer was removed, and the organic layer washed with brine, dried over anhydrous magnesium sulfate, filtered and concentrated to provide the title compound, which was used in the next step without further purification. ¹H NMR (500 MHz, dimethyl sulfoxide-d₆) δ ppm 2.53 (s, 3H), 7.60 (d, 1H), 7.79 (d, 1H), 10.32 (s, 1H).

### Example 10B

### tert-butyl 4-bromo-2-chloro-3-methylbenzyl(2-(4-methylpiperazin-1-yl)ethyl)carbamate

To a mixture of Example 10A (265 mg) in dichloromethane (12 mL) with 2-(4-methylpiperazin-1-yl)ethanamine (195 mg) was added acetic acid (0.325 mL), sodium cyanoborohydride (143 mg) and methanol (3.03 mL). The mixture was stirred at ambient temperature for 30 minutes, and di-tert-butyl dicarbonate (0.395 mL) was added. Stirring was continued for two additional hours. Triethylamine (1 mL) was added. The material was dissolved following methanol addition (5 mL). The mixture was concentrated onto silica gel and purification by silica gel chromatography on a CombiFlash® Teledyne Isco system using a Teledyne Isco RediSep® Rf gold 24 g silica gel column (eluting with solvent A=2:1 ethyl acetate:ethanol with 3% triethylamine; solvent B=3% triethylamine in heptane; 0-100% A to B) provided the title compound. LC/MS (APCI) m/z 462.2 (M+H)⁺.

### Example 10C

### tert-butyl 2-chloro-3-methyl-4-(4,4,5,5-tetramethyl-1,3,2-dioxaborolan-2-yl)benzyl(2-(4-methylpiperazin-1-yl)ethyl)carbamate

The title compound was prepared as described in Example 7H substituting Example 10B for Example 7G. LC/MS (APCI) m/z 508.4 (M+H)⁺.

### Example 10D

### (R)-ethyl 3-(5-(2-(tert-butoxy)-2-oxoethyl)-2-((2-(3,3,3-trifluoropropoxy)pyrimidin-4-yl)methoxy)phenyl)-2-((5-((1S)-4-(((tert-butoxycarbonyl)(2-(4-methylpiperazin-1-yl)ethyl)amino)methyl)-3-chloro-2-methylphenyl)-6-(4-fluorophenyl)thieno[2,3-d]pyrimidin-4-yl)oxy)propanoate

The title compound was prepared as described in Example 7N substituting Example 10C for Example 7H. LC/MS (APCI) m/z 1136.4 (M+H)⁺.

### Example 10E

### ethyl (7R,21S)-19-chloro-1-(4-fluorophenyl)-20-methyl-16-[2-(4-methylpiperazin-1-yl)ethyl]-15-oxo-10-{[2-(3,3,3-trifluoropropoxy)pyrimidin-4-yl]methoxy}-7,8,14,15,16,17-hexahydro-18,21-etheno-13,9-(metheno)-6-oxa-2-thia-3,5,16-triazacyclononadeca[1,2,3-cd]indene-7-carboxylate

Example 10D (74 mg) was dissolved in 1 mL of dichloromethane and was treated with 1 mL of trifluoroacetic acid. The mixture was stirred at ambient temperature for 10 minutes and was concentrated. The residue was dissolved in dichloromethane (6.5 mL) and 1-[bis(dimethylamino)methylene]-1H-1,2,3-triazolo[4,5-b]pyridinium 3-oxid hexafluorophosphate (37.1 mg, HATU), 1-hydroxybenzotriazole hydrate (7.5 mg), 4-dimethylaminopyridine (0.8 mg) and N,N-diisopropylethylamine (0.23 mL) were added successively. The reaction mixture was stirred at room temperature for 24 hours. The mixture was concentrated onto silica gel and purification by silica gel chromatography on a CombiFlash® Teledyne Isco system using a Teledyne Isco RediSep® Rf gold 12 g silica gel column (eluting with solvent A=2:1 methanol:water; solvent B=ethyl acetate; 0-50% A to B) provided the title compound. LC/MS (APCI) m/z 962.3 (M+H)⁺.

### Example 10F

### (7R,21S)-19-chloro-1-(4-fluorophenyl)-20-methyl-16-[2-(4-methylpiperazin-1-yl)ethyl]-15-oxo-10-{[2-(3,3,3-trifluoropropoxy)pyrimidin-4-yl]methoxy}-7,8,14,15,16,17-hexahydro-18,21-etheno-13,9-(metheno)-6-oxa-2-thia-3,5,16-triazacyclononadeca[1,2,3-cd]indene-7-carboxylic acid

Example 10E (43.3 mg) was dissolved in tetrahydrofuran (0.6 mL), and 1 molar aqueous lithium hydroxide (0.6 mL) was added followed by 0.25 mL of methanol. The mixture was stirred at ambient temperature for 4 hours. The mixture was concentrated to remove the volatiles, and the resulting aqueous mixture was acidified with trifluoroacetic acid until the pH approximated 1. The precipitate that formed was redissolved by adding 1 mL of acetonitrile. The resulting mixture was purified directly by Gilson reverse-phase prep HPLC (Zorbax, C-18, 250×21.2 mm column, mobile phase A: 0.1% trifluoroacetic acid in water; B: 0.1% trifluoroacetic acid in acetonitrile; 10-100% B to A gradient) to provide the title compound. ¹H NMR (400 MHz, dimethyl sulfoxide-d₆) δ ppm 1.82 (s, 3H), 2.66-2.77 (m, 5H), 2.79-2.91 (m, 5H), 3.10-3.18 (m, 5H), 3.20-3.36 (m, 2H), 3.44 (d, 1H), 3.73-3.86 (m, 1H), 4.09-4.20 (m, 1H), 4.42 (d, 1H), 4.48-4.54 (m, 2H), 4.67-4.83 (m, 2H), 4.87-4.96 (m, 1H), 5.53-5.63 (m, 1H), 6.51 (d, 1H), 6.72 (d, 1H), 6.83 (d, 1H), 6.87 (d, 1H), 7.01-7.11 (m, 5H), 7.20-7.28 (m, 2H), 8.41 (d, 1H), 8.47 (s, 1H). LC/MS (APCI) m/z 934.1 (M+H)⁺.

### Example 11

### (7R,21S)-19-chloro-1-(4-fluorophenyl)-10-{[2-(2-methoxyphenyl)pyrimidin-4-yl]methoxy}-20-methyl-16-[2-(4-methylpiperazin-1-yl)ethyl]-15-oxo-7,8,14,15,16,17-hexahydro-18,21-etheno-13,9-(metheno)-6-oxa-2-thia-3,5,16-triazacyclononadeca[1,2,3-cd]indene-7-carboxylic acid

### Example 11A

### (R)-ethyl 2-acetoxy-3-(5-(2-(tert-butoxy)-2-oxoethyl)-2-((2-(2-methoxyphenyl)pyrimidin-4-yl)methoxy)phenyl)propanoate

A mixture of Example 1L (2.65 g), 2-tert-butoxy-2-oxoethylzinc chloride (0.5 molar in diethyl ether; 12 mL), tris(dibenzylidenacetone)dipalladium(0) (0.275 g) and 1,2,3,4,5-pentaphenyl-1′-(di-tert-butylphosphino)ferrocene (0.355 g, QPHOS) in anhydrous tetrahydrofuran (14.7 mL) was degassed by bubbling nitrogen through the mixture for 3 minutes. The mixture was stirred at 70° C. for 90 minutes. After cooling to ambient temperature, the mixture was poured into a separatory funnel, and was diluted with ethyl acetate. The layers were separated, and the organic mixture was washed with water and saturated aqueous brine, dried over anhydrous magnesium sulfate, filtered and concentrated onto silica gel. Purification by silica gel chromatography on a CombiFlash® Teledyne Isco system using a Teledyne Isco RediSep® Rf gold 24 g silica gel column (eluting with 10-75% ethyl acetate/heptane) provided the title compound. LC/MS (APCI) m/z 565.3 (M+H)⁺.

### Example 11B

### (R)-ethyl 3-(5-(2-(tert-butoxy)-2-oxoethyl)-2-((2-(2-methoxyphenyl)pyrimidin-4-yl)methoxy)phenyl)-2-hydroxypropanoate

The title compound was prepared as described in Example 7L, substituting Example 11A for Example 7K. LC/MS (APCI) m/z 523.2 (M+H)⁺.

### Example 11C

### (R)-ethyl 2-((5-bromo-6-(4-fluorophenyl)thieno[2,3-d]pyrimidin-4-yl)oxy)-3-(5-(2-(tert-butoxy)-2-oxoethyl)-2-((2-(2-methoxyphenyl)pyrimidin-4-yl)methoxy)phenyl)propanoate

The title compound was prepared as described in Example 7M, substituting Example 11B for Example 7L. LC/MS (APCI) m/z 831.1 (M+H)⁺.

### Example 11D

### (R)-ethyl 3-(5-(2-(tert-butoxy)-2-oxoethyl)-2-((2-(2-methoxyphenyl)pyrimidin-4-yl)methoxy)phenyl)-2-((5-((1S)-4-(((tert-butoxycarbonyl)(2-(4-methylpiperazin-1-yl)ethyl)amino)methyl)-3-chloro-2-methylphenyl)-6-(4-fluorophenyl)thieno[2,3-d]pyrimidin-4-yl)oxy)propanoate

The title compound was prepared as described in Example 7N, substituting Example 11C for Example 7M and substituting Example 10C for Example 7H. LC/MS (APCI) m/z 1130.4 (M+H)⁺.

### Example 11E

### ethyl (7R,21S)-19-chloro-1-(4-fluorophenyl)-10-{[2-(2-methoxyphenyl)pyrimidin-4-yl]methoxy}-20-methyl-16-[2-(4-methylpiperazin-1-yl)ethyl]-15-oxo-7,8,14,15,16,17-hexahydro-18,21-etheno-13,9-(metheno)-6-oxa-2-thia-3,5,16-triazacyclononadeca[1,2,3-cd]indene-7-carboxylate

The title compound was prepared as described in Example 10E, substituting Example 11D for Example 10D. LC/MS (APCI) m/z 956.3 (M+H)⁺.

### Example 11F

### (7R,21S)-19-chloro-1-(4-fluorophenyl)-10-{[2-(2-methoxyphenyl)pyrimidin-4-yl]methoxy}-20-methyl-16-[2-(4-methylpiperazin-1-yl)ethyl]-15-oxo-7,8,14,15,16,17-hexahydro-18,21-etheno-13,9-(metheno)-6-oxa-2-thia-3,5,16-triazacyclononadeca[1,2,3-cd]indene-7-carboxylic acid

The title compound was prepared as described in Example 10F, substituting Example 11E for Example 10E. ¹H NMR (120° C.) (400 MHz, dimethyl sulfoxide-d₆) δ ppm 1.82 (s, 3H), 2.74 (s, 3H), 2.81-2.95 (m, 5H), 3.10-3.21 (m, 4H), 3.23-3.42 (m, 2H), 3.45 (d, 1H), 3.74 (s, 3H), 3.76-3.86 (m, 1H), 4.09-4.21 (m, 1H), 4.42 (d, 1H), 4.77-4.99 (m, 3H), 5.60-5.65 (m, 1H), 6.51 (d, 1H), 6.77 (d, 1H), 6.84 (d, 1H), 6.99-7.13 (m, 7H), 7.18-7.26 (m, 2H), 7.35-7.45 (m, 1H), 7.51-7.58 (m, 1H), 8.49 (s, 1H), 8.66 (d, 1H). LC/MS (APCI) m/z 928.3 (M+H)⁺.

### Example 12

### (7R,21R)-19-chloro-1-(4-fluorophenyl)-10-{[2-(2-methoxyphenyl)pyrimidin-4-yl]methoxy}-20-methyl-16-[2-(4-methylpiperazin-1-yl)ethyl]-15-oxo-7,8,14,15,16,17-hexahydro-18,21-etheno-13,9-(metheno)-6-oxa-2-thia-3,5,16-triazacyclononadeca[1,2,3-cd]indene-7-carboxylic acid

The title compound was obtained during the synthesis of Example 11F and was isolated by Gilson reverse-phase prep HPLC (Zorbax, C-18, 250×2.54 column, Mobile phase A: 0.1% trifluoroacetic acid in water; B: 0.1% trifluoroacetic acid in acetonitrile; 10-100% B to A gradient). ¹H NMR (400 MHz, dimethyl sulfoxide-d₆) δ ppm 2.25 (s, 3H), 2.55 (dd, 1H), 2.69-2.79 (m, 5H), 2.79-2.89 (m, 4H), 2.96-3.08 (m, 1H), 3.08-3.18 (m, 4H), 3.37-3.49 (m, 2H), 3.74 (s, 3H), 3.79 (d, 1H), 3.97-4.09 (m, 1H), 4.48-4.57 (m, 1H), 4.88 (d, 1H), 5.00-5.17 (m, 2H), 6.16 (dd, 1H), 6.20-6.28 (m, 1H), 6.40 (d, 1H), 6.46 (d, 1H), 6.82 (d, 1H), 6.98-7.08 (m, 3H), 7.08-7.15 (m, 3H), 7.18-7.26 (m, 2H), 7.37-7.45 (m, 1H), 7.53 (dt, 1H), 8.44 (s, 1H), 8.55-8.63 (m, 1H). LC/MS (APCI) m/z 928.3 (M+H)⁺.

### Example 13

### (7R,20S)-18-chloro-1-(4-fluorophenyl)-10-{[2-(2-methoxyphenyl)pyrimidin-4-yl]methoxy}-19-methyl-16-[2-(4-methylpiperazin-1-yl)ethyl]-15-oxo-7,8,15,16-tetrahydro-14H-17,20-etheno-13,9-(metheno)-6-oxa-2-thia-3,5,16-triazacyclooctadeca[1,2,3-cd]indene-7-carboxylic acid

### Example 13A

### (R)-ethyl 2-((5-((1S)-4-amino-3-chloro-2-methylphenyl)-6-(4-fluorophenyl)thieno[2,3-d]pyrimidin-4-yl)oxy)-3-(5-(2-(tert-butoxy)-2-oxoethyl)-2-((2-(2-methoxyphenyl)pyrimidin-4-yl)methoxy)phenyl)propanoate

The title compound was prepared as described in Example 7N, substituting Example 11C for Example 7M. LC/MS (APCI) m/z 890.3 (M+H)⁺.

### Example 13B

### 2-(3-((R)-2-((5-((1S)-4-amino-3-chloro-2-methylphenyl)-6-(4-fluorophenyl)thieno[2,3-d]pyrimidin-4-yl)oxy)-3-ethoxy-3-oxopropyl)-4-((2-(2-methoxyphenyl)pyrimidin-4-yl)methoxy)phenyl)acetic acid

The title compound was prepared as described in Example 7O, substituting Example 13A for Example 7N. LC/MS (APCI) m/z 834.2 (M+H)⁺.

### Example 13C

### ethyl (7R,20S)-18-chloro-1-(4-fluorophenyl)-10-{[2-(2-methoxyphenyl)pyrimidin-4-yl]methoxy}-19-methyl-15-oxo-7,8,15,16-tetrahydro-14H-17,20-etheno-13,9-(metheno)-6-oxa-2-thia-3,5,16-triazacyclooctadeca[1,2,3-cd]indene-7-carboxylate

The title compound was prepared as described in Example 7P, substituting Example 13B for Example 7O. LC/MS (APCI) m/z 816.2 (M+H)⁺.

### Example 13D

### ethyl (7R,20S)-16-{2-[4-(tert-butoxycarbonyl)piperazin-1-yl]ethyl}-18-chloro-1-(4-fluorophenyl)-10-{[2-(2-methoxyphenyl)pyrimidin-4-yl]methoxy}-19-methyl-15-oxo-7,8,15,16-tetrahydro-14H-17,20-etheno-13,9-(metheno)-6-oxa-2-thia-3,5,16-triazacyclooctadeca[1,2,3-cd]indene-7-carboxylate

The title compound was prepared as described in Example 7Q, substituting Example 13C for Example 7P. LC/MS (APCI) m/z 1028.4 (M+H)⁺.

### Example 13E

### (7R,20S)-18-chloro-1-(4-fluorophenyl)-10-{[2-(2-methoxyphenyl)pyrimidin-4-yl]methoxy}-19-methyl-16-[2-(4-methylpiperazin-1-yl)ethyl]-15-oxo-7,8,15,16-tetrahydro-14H-17,20-etheno-13,9-(metheno)-6-oxa-2-thia-3,5,16-triazacyclooctadeca[1,2,3-cd]indene-7-carboxylic acid

The title compound was prepared as described in Example 9, substituting Example 13D for Example 7Q. ¹H NMR (400 MHz, dimethyl sulfoxide-d₆) δ ppm 2.12 (s, 3H), 2.75 (s, 5H), 2.96-3.52 (m, 12H), 3.64-3.74 (m, 1H), 3.74 (s, 3H), 4.31 (dt, 1H), 5.18-5.29 (m, 2H), 5.93 (d, 1H), 6.41 (t, 1H), 6.94 (d, 1H), 6.99-7.08 (m, 2H), 7.08-7.20 (m, 3H), 7.22-7.30 (m, 2H), 7.38-7.44 (m, 2H), 7.46 (d, 1H), 7.53 (dd, 2H), 8.75 (s, 1H), 8.84 (d, 1H). LC/MS (APCI) m/z 914.3 (M+H)⁺.

### Example 14

### (7R)-18-chloro-1-(4-fluorophenyl)-10-{[2-(2-methoxyphenyl)pyrimidin-4-yl]methoxy}-19-methyl-15-oxo-7,8,15,16-tetrahydro-14H-17,20-etheno-13,9-(metheno)-6-oxa-2-thia-3,5,16-triazacyclooctadeca[1,2,3-cd]indene-7-carboxylic acid

The title compound was prepared as described in Example 10F, substituting Example 13C for Example 10E. ¹H NMR (500 MHz, dimethyl sulfoxide-d₆) δ ppm 2.17 (s, 3H), 3.01 (dd, 1H), 3.12 (d, 1H), 3.35-3.44 (m, 1H), 3.51-3.57 (m, 4H), 3.78 (s, 3H), 5.17-5.30 (m, 2H), 5.92 (s, 1H), 6.33 (t, 1H), 6.96 (d, 1H), 6.98-7.29 (m, 6H), 7.30-7.40 (m, 3H), 7.42-7.50 (m, 2H), 7.57 (d, 1H), 8.77 (s, 1H), 8.87 (d, 1H), 9.21 (s, 1H). LC/MS (APCI) m/z 788.1 (M+H)⁺.

### Example 15

### (7R,20S)-18-chloro-1-(4-fluorophenyl)-10-{[2-(2-methoxyphenyl)pyrimidin-4-yl]methoxy}-19-methyl-16-[3-(4-methylpiperazin-1-yl)propyl]-15-oxo-7,8,15,16-tetrahydro-14H-17,20-etheno-13,9-(metheno)-6-oxa-2-thia-3,5,16-triazacyclooctadeca[1,2,3-cd]indene-7-carboxylic acid

### Example 15A

### ethyl (7R,20S)-18-chloro-1-(4-fluorophenyl)-10-{[2-(2-methoxyphenyl)pyrimidin-4-yl]methoxy}-19-methyl-16-[3-(4-methylpiperazin-1-yl)propyl]-15-oxo-7,8,15,16-tetrahydro-14H-17,20-etheno-13,9-(metheno)-6-oxa-2-thia-3,5,16-triazacyclooctadeca[1,2,3-cd]indene-7-carboxylate

The title compound was prepared as described in Example 7Q, substituting Example 13C for Example 7P and substituting 3-(N-methylpiperazine)propyl bromide dihydrobromide for tert-butyl 4-(2-bromomethyl)piperazine-1-carboxylate. LC/MS (APCI) m/z 956.3 (M+H)⁺.

### Example 15B

### (7R,20S)-18-chloro-1-(4-fluorophenyl)-10-{[2-(2-methoxyphenyl)pyrimidin-4-yl]methoxy}-19-methyl-16-[3-(4-methylpiperazin-1-yl)propyl]-15-oxo-7,8,15,16-tetrahydro-14H-17,20-etheno-13,9-(metheno)-6-oxa-2-thia-3,5,16-triazacyclooctadeca[1,2,3-cd]indene-7-carboxylic acid

The title compound was prepared as described in Example 10F, substituting Example 15A for Example 10E. ¹H NMR (400 MHz, dimethyl sulfoxide-d₆) δ ppm 1.64-1.79 (m, 2H), 2.12 (s, 3H), 2.82 (s, 3H), 2.88-3.63 (m, 14H), 3.66-3.73 (m, 1H), 3.74 (s, 3H), 4.11 (dt, 1H), 5.23 (s, 2H), 5.95 (d, 1H), 6.41 (t, 1H), 6.94 (d, 1H), 6.98-7.09 (m, 2H), 7.09-7.19 (m, 4H), 7.22-7.30 (m, 2H), 7.34-7.49 (m, 3H), 7.53 (dd, 1H), 8.75 (s, 1H), 8.84 (d, 1H). LC/MS (APCI) m/z 928.2 (M+H)⁺.

### Example 16

### (7R,21S)-19-chloro-1-(4-fluorophenyl)-20-methyl-17-[2-(4-methylpiperazin-1-yl)ethyl]-16-oxo-10-{[2-(3,3,3-trifluoropropoxy)pyrimidin-4-yl]methoxy}-7,8,16,17-tetrahydro-15H-18,21-etheno-13,9-(metheno)-6,14-dioxa-2-thia-3,5,17-triazacyclononadeca[1,2,3-cd]indene-7-carboxylic acid

### Example 16A

### 2-(benzyloxy)-5-((tert-butyldimethylsilyl)oxy)benzaldehyde

A 2 L round bottom flask was charged with 2,5-dihydroxybenzaldehyde (30 g), imidazole (29.6 g) and dichloromethane (543 mL). The flask was placed in a water bath and solid tert-butylchlorodimethylsilane (32.7 g) was added. The reaction mixture was stirred at ambient temperature for 15 minutes at which point thin-layer chromatography indicated complete consumption of starting material. The reaction mixture was poured into a separatory funnel with 200 mL water. The biphasic mixture was shaken and layers were separated. The aqueous layer was washed with 100 mL dichloromethane and the organic layers were combined. The organic layer was dried over sodium sulfate, filtered, and concentrated and the material was used in the next step. A 1 L three-necked round bottom flask equipped with an internal temperature probe, a reflux condenser, and a stir bar was charged with 5-((tert-butyldimethylsilyl)oxy)-2-hydroxybenzaldehyde (45 g, 178 mmol) in acetone (297 mL). Solid K₂CO₃ (27.1 g) was added followed by dropwise addition of neat benzyl bromide (21.21 mL). The mixture was stirred at ambient temperature for 10 minutes and heated to 55° C. The reaction mixture stirred overnight. The reaction mixture was cooled to ambient temperature then poured over cold water (200 mL). The mixture was then transferred to a 1 L separatory funnel. The crude product was extracted with ethyl acetate (3×250 mL). The combined organic layers were dried over sodium sulfate, filtered, and concentrated. The crude material was purified by silica gel chromatography over a 330 g column on a Grace Reveleris system (0-5% ethyl acetate/heptanes elution gradient). Fractions containing the desired product were combined, concentrated and dried under vacuum to obtain the title compound. ¹H NMR (501 MHz, dimethyl sulfoxide-d₆) δ ppm 10.35 (s, 1H), 7.51-7.47 (m, 2H), 7.42-7.37 (m, 2H), 7.35-7.31 (m, 1H), 7.22 (d, 1H), 7.15 (dd, 1H), 7.11 (d, 1H), 5.21 (s, 2H), 0.93 (s, 10H), 0.16 (s, 7H).

### Example 16B

### (E)/(Z)-ethyl 2-acetoxy-3-(2-(benzyloxy)-5-((tert-butyldimethylsilyl)oxy)phenyl)acrylate

Into a 50 mL Erlenmeyer flask ethyl 2-acetoxy-2-(diethoxyphosphoryl)acetate (37.1 g) was weighed and dried over anhydrous MgSO₄. The mixture was filtered over a 0.5 inch bed of silica and washed with toluene (50 mL) into a 1 L round bottom flask. The toluene mixture was concentrated and 200 mL tetrahydrofuran was added followed by Cs₂CO₃ (42.8 g). The mixture was stirred at ambient temperature for 20 minutes. A tetrahydrofuran mixture (15 mL+50 mL washing) of Example 16A (15 g) was added, and the reaction mixture was stirred at ambient temperature for 66 hours. The reaction mixture was filtered, and the filtrate was transferred to a separatory funnel with 200 mL water. The layers were separated. The aqueous layer was washed with ethyl acetate (2×100 mL), and the combined organic layers were washed with brine, dried over MgSO₄, filtered, and concentrated. The crude material was purified by silica gel chromatography over a 330 g column on a Grace Reveleris system (0-10% ethyl acetate/heptanes elution gradient). Fractions containing the desired product were combined, concentrated and dried under vacuum to obtain the title compound as an inseparable E/Z mixture. The E/Z ratio was found to be inconsequential for the subsequent step. ¹H NMR of Z isomer (tentatively assigned): ¹H NMR (400 MHz, dimethyl sulfoxide-d₆) δ ppm 7.63 (s, 1H), 7.48-7.32 (m, 5H), 7.15 (d, 1H), 7.10 (d, 1H), 6.92 (dd, 1H), 5.13 (s, 2H), 4.20 (q, 2H), 2.27 (s, 3H), 1.23 (t, 3H), 0.94 (s, 9H), 0.16 (s, 6H). ¹H NMR of E isomer (tentatively assigned): ¹H NMR (400 MHz, dimethyl sulfoxide-d₆) δ ppm 7.48-7.29 (m, 5H), 6.98 (d, 1H), 6.88 (s, 1H), 6.80 (d, 2H), 5.05 (s, 2H), 4.02 (q, 2H), 2.20 (s, 3H), 1.03 (t, 3H), 0.94 (s, 9H), 0.15 (s, 6H). MS (ESI) m/z 488.0 (M+NH₄)⁺.

### Example 16C

### (R)-ethyl 2-acetoxy-3-(2-(benzyloxy)-5-((tert-butyldimethylsilyl)oxy)phenyl)propanoate

A 100 mL Parr stainless steel reactor was charged with degassed methanol (37.5 mL) and Example 16B (10.5 g). In a nitrogen-filled glove box, a vial was charged with (1,2-Bis[(2R,5R)-2,5-diethylphospholano]benzene(1,5-cyclooctadiene)rhodium(I) trifluoromethanesulfonate (0.45 g) and degassed methanol (4 mL) was added. The catalyst mixture was capped and brought outside the glove box and added to the reactor via syringe. The reaction mixture was stirred under 50 psi of hydrogen at 35° C. for 8 hours. The reaction mixture was cooled to ambient temperature and filtered. The filtrate was concentrated. The crude material was purified on a silica plug with 20% ethyl acetate/heptanes as the eluent. The fractions containing the desired product were combined and concentrated to obtain the title compound. ¹H NMR (500 MHz, dimethyl sulfoxide-d₆) δ ppm 7.48-7.43 (m, 2H), 7.41-7.36 (m, 2H), 7.35-7.29 (m, 1H), 6.93 (dt, 1H), 6.72-6.66 (m, 2H), 5.12 (dd, 1H), 5.09-5.00 (m, 2H), 4.03 (qd, 2H), 3.16 (dd, 1H), 2.96 (dd, 1H), 1.97 (s, 3H), 1.07 (t, 3H), 0.93 (s, 9H), 0.14 (s, 6H). MS (DCI) m/z 490.2 (M+NH₄)⁺. Enantiomeric excess was determined in the following way: A vial was charged with Example 16C (8 mg) and tetrahydrofuran (1 mL). A 1 M mixture of tetrabutyl ammonium fluoride was added in a single portion. After 5 minutes, the reaction mixture was diluted with ethyl acetate (1 mL) and poured over water (1 mL). The biphasic mixture was vigorously stirred, the layers were allowed to separate, and the organic layer was removed via a pipette. The organic layer was dried over MgSO₄, filtered, and concentrated. Analytical SFC: 5-50% methanol, ChiralPak IC column, retention time for the R enantiomer=2.28 minutes, retention time for the S enantiomer=2.08 minutes. The enantiomeric excess of the sample was determined to be >99%.

### Example 16D

### (R)-ethyl 2-acetoxy-3-(5-((tert-butyldimethylsilyl)oxy)-2-hydroxyphenyl)propanoate

Example 16C (10.2 g) in ethanol (70 mL) was added to 5% Pd/C (wet JM #9) (0.517 g) in a 250 mL pressure bottle. The mixture was stirred under 50 psi of hydrogen (g) at 35° C. for 7.5 hours. The reaction mixture was cooled to ambient temperature and filtered. The filtrate was concentrated to obtain the title compound. ¹H NMR (400 MHz, dimethyl sulfoxide-d₆) δ ppm 9.08 (s, 1H), 6.68-6.60 (m, 1H), 6.59-6.49 (m, 2H), 5.09 (dd, 1H), 4.05 (q, 2H), 3.02 (dd, 1H), 2.87 (dd, 1H), 1.99 (s, 3H), 1.11 (t, 3H), 0.92 (s, 9H), 0.11 (s, 6H). MS (ESI) m/z 399.8 (M+NH₄)⁺. Analytical SFC: 5-50% methanol, Whelk-O1 (S,S) column, retention time for the R enantiomer=1.828 minutes, retention time for the S enantiomer=1.926 minutes. The enantiomeric excess of the sample was determined to be >99%.

### Example 16E

### ethyl (R)-2-acetoxy-3-(5-((tert-butyldimethylsilyl)oxy)-2-((2-(3,3,3-trifluoropropoxy)pyrimidin-4-yl)methoxy)phenyl)propanoate

To an oven dried 500 mL round bottom flask was added Example 16D (8 g), triphenylphosphine (10.97 g), Example 7E (5.58 g) and tetrahydrofuran (105 mL). The reaction mixture was placed in an ice bath. When the reaction was cooled to 3° C. internal temperature, solid (E)-N,N,N′,N′-tetramethyldiazene-1,2-dicarboxamide (7.20 g) was added (no exotherm observed) and the reaction mixture was allowed to warm up to ambient temperature overnight. After about 2 minutes, a precipitate was observed. The next morning thin-layer chromatography indicated complete consumption of starting material. The reaction mixture was transferred to a 500 mL single-necked round bottom flask and concentrated. Ethyl acetate (100 mL) was added and the mixture was stirred for about 30 minutes and filtered. The filtrate was concentrated and the crude material was purified on Grace Reveleris system using a 220 g silica column using 0-25% ethyl acetate/heptanes. Fractions containing pure product were combined and concentrated to obtain the title compound. ¹H NMR (400 MHz, dimethyl sulfoxide-d₆) δ ppm 8.66 (d, 1H), 7.30 (d, 1H), 6.89 (d, 1H), 6.73 (d, 1H), 6.69 (dd, 1H), 5.14 (dd, 1H), 5.09 (d 2H), 4.52 (t2H), 4.06 (qd, 2H), 3.23 (dd, 1H), 3.02 (dd1H), 2.81 (qt, 2H), 1.99 (s, 3H), 1.10 (t, 3H), 0.93 (s, 9H), 0.14 (s, 6H). MS (ESI) m/z 387.1 (M+H)⁺.

### Example 16F

### ethyl (R)-3-(5-((tert-butyldimethylsilyl)oxy)-2-((2-(3,3,3-trifluoropropoxy)pyrimidin-4-yl)methoxy)phenyl)-2-hydroxypropanoate

To a mixture of Example 16E (3.2 g) in ethanol (60 mL) was added anhydrous potassium carbonate (3.015 g), and the mixture was stirred at room temperature and was monitored by LC/MS. After 2 hours, LC/MS showed complete consumption of starting material with a major peak consistent with the desired product. The mixture was poured into water (100 mL), and the mixture was extracted with three portions of ethyl acetate. The combined organics were dried over anhydrous magnesium sulfate, filtered and concentrated. The crude product was used in the next step without purification. LC/MS (APCI) m/z 545.0 (M+H)⁺.

### Example 16G

### (R)-ethyl 2-((5-bromo-6-(4-fluorophenyl)thieno[2,3-d]pyrimidin-4-yl)oxy)-3-(5-((tert-butyldimethylsilyl)oxy)-2-((2-(3,3,3-trifluoropropoxy)pyrimidin-4-yl)methoxy)phenyl)propanoate

To a 250 mL round-bottom flask containing Example 16F (2.97 g) were added Example 1D (1.873 g), cesium carbonate (5.33 g) and tert-butanol (50 mL). The flask was capped, and the mixture was stirred at 65° C. for 2 hours. The mixture was poured into a separatory funnel and was diluted with ethyl acetate. The mixture was washed with water and brine, dried over anhydrous sodium sulfate, filtered and concentrated. The residue was purified by silica gel chromatography on an AnaLogix IntelliFlash²⁸⁰ system (0-30% ethyl acetate/heptanes, linear gradient) to provide the title compound. LC/MS (APCI) m/z 853.2 (M+H)⁺.

### Example 16H

### (R)-ethyl 2-((5-bromo-6-(4-fluorophenyl)thieno[2,3-d]pyrimidin-4-yl)oxy)-3-(5-hydroxy-2-((2-(3,3,3-trifluoropropoxy)pyrimidin-4-yl)methoxy)phenyl)propanoate

Example 16G (2.440 g) was taken up in tetrahydrofuran (24 mL) at room temperature under nitrogen. Tetrabutylammonium fluoride (5.73 mL, 1.0 M in tetrahydrofuran) was added dropwise. The mixture was stirred at room temperature for 1 day. The reaction mixture was poured into a separatory funnel and was diluted with ethyl acetate and 1:1 water:saturated NH₄Cl mixture. The layers were separated, and the aqueous layer was extracted with ethyl acetate. The combined organics were dried over anhydrous sodium sulfate, filtered and concentrated. The residue was purified by silica gel chromatography on an AnaLogix IntelliFlash²⁸⁰ system (0-30% ethyl acetate in hexanes, linear gradient) to provide the title compound. LC/MS (APCI) m/z 739.2 (M+H)⁺.

### Example 16I

### (R)-ethyl 2-((5-bromo-6-(4-fluorophenyl)thieno[2,3-d]pyrimidin-4-yl)oxy)-3-(5-(2-(tert-butoxy)-2-oxoethoxy)-2-((2-(3,3,3-trifluoropropoxy)pyrimidin-4-yl)methoxy)phenyl)propanoate

Example 16H (1000 mg) with cesium carbonate (884 mg) in N,N-dimethylformamide (9 mL) was stirred vigorously at 0° C. and was treated with tert-butyl bromoacetate (0.238 mL). The cooling bath was removed, and the mixture was stirred at ambient temperature temperature for 1 hour. The mixture was poured into a separatory funnel and was diluted with ethyl acetate. The mixture was washed with water (twice) and brine, dried over anhydrous sodium sulfate, filtered and concentrated. The residue was purified by silica gel chromatography on an AnaLogix IntelliFlash²⁸⁰ system (0-30% ethyl acetate/heptane, linear gradient) to provide the title compound. LC/MS (APCI) m/z 853.3 (M+H)⁺.

### Example 16J

### (R)-ethyl 2-((5-((1S)-4-amino-3-chloro-2-methylphenyl)-6-(4-fluorophenyl)thieno[2,3-d]pyrimidin-4-yl)oxy)-3-(5-(2-(tert-butoxy)-2-oxoethoxy)-2-((2-(3,3,3-trifluoropropoxy)pyrimidin-4-yl)methoxy)phenyl)propanoate

Example 16I (300 mg), Example 7H (123 mg), bis(di-tert-butyl(4-dimethylaminophenyl)phosphine)dichloropalladium(II) (24.94 mg) and cesium carbonate (344 mg) were placed in a 25 mL pressure vial, and the reaction mixture was degassed and purged with nitrogen. Tetrahydrofuran (3.0 mL) and water (0.75 mL) were added via syringe, and the reaction mixture was degassed and purged with nitrogen. The reaction mixture was heated to 40° C. for 3 hours. To the mixture was added water, and the mixture was extracted with ethyl acetate. The organics were dried over anhydrous sodium sulfate, filtered, and concentrated. The residue was purified with flash chromatography purification on an AnaLogix IntelliFlash²⁸⁰ system (5-50% ethyl acetate in hexanes, linear gradient) to provide the title compound. LC/MS (APCI) m/z 912.2 (M+H)⁺.

### Example 16K

### (3-[(2R)-2-{[5-((1S)-4-amino-3-chloro-2-methylphenyl)-6-(4-fluorophenyl)thieno[2,3-d]pyrimidin-4-yl]oxy}-3-ethoxy-3-oxopropyl]-4-{[2-(3,3,3-trifluoropropoxy)pyrimidin-4-yl]methoxy}phenoxy)acetic acid

Example 16J (80 mg) was dissolved in dichloromethane (0.5 mL), and 0.5 mL of trifluoroacetic acid was added. After 3 hours, the mixture was concentrated. The crude product was used in the next step without further purification. LC/MS (APCI) m/z 856.2 (M+H)⁺.

### Example 16L

### ethyl (7R,21S)-19-chloro-1-(4-fluorophenyl)-20-methyl-16-oxo-10-{[2-(3,3,3-trifluoropropoxy)pyrimidin-4-yl]methoxy}-7,8,16,17-tetrahydro-15H-18,21-etheno-13,9-(metheno)-6,14-dioxa-2-thia-3,5,17-triazacyclononadeca[1,2,3-cd]indene-7-carboxylate

Example 16K (51.4 mg) was dissolved in dichloromethane (6 mL). 1-[Bis(dimethylamino)methylene]-1H-1,2,3-triazolo[4,5-b]pyridinium-3-oxid hexafluorophosphate (34.2 mg, HATU), 1-hydroxybenzotriazole hydrate (6.89 mg), 4-dimethylaminopyridine (7.3 mg) and N,N-diisopropylethylamine (0.062 mL) were added. The reaction mixture was stirred at ambient temperature for 2 days. The mixture was diluted with ethyl acetate and washed with water. The organics were separated, dried over anhydrous sodium sulfate, filtered, and concentrated. The residue was purified by silica gel chromatography on an AnaLogix IntelliFlash²⁸⁰ system (10-100% ethyl acetate/heptanes, linear gradient) to provide the title compound. LC/MS (APCI) m/z 838.1 (M+H)⁺.

### Example 16M

### ethyl (7R,21S)-17-{2-[4-(tert-butoxycarbonyl)piperazin-1-yl]ethyl}-19-chloro-1-(4-fluorophenyl)-20-methyl-16-oxo-10-{[2-(3,3,3-trifluoropropoxy)pyrimidin-4-yl]methoxy}-7,8,16,17-tetrahydro-15H-18,21-etheno-13,9-(metheno)-6,14-dioxa-2-thia-3,5,17-triazacyclononadeca[1,2,3-cd]indene-7-carboxylate

Example 16L (67.1 mg) was dissolved in N,N-dimethylformamide (0.8 mL). tert-Butyl 4-(2-bromoethyl)piperazine-1-carboxylate (35.2 mg) and cesium carbonate (78.0 mg) were added. The reaction mixture was stirred at ambient temperature for 40 minutes. The mixture was diluted with ethyl acetate and water. The organics were separated, dried over anhydrous sodium sulfate, filtered, and concentrated. The residue was purified by silica gel chromatography on an AnaLogix IntelliFlash²⁸⁰ system (50-100% ethyl acetate/heptanes, linear gradient) to provide the title compound. LC/MS (APCI) m/z 1050.3 (M+H)⁺.

### Example 16N

### ethyl (7R,21S)-19-chloro-1-(4-fluorophenyl)-20-methyl-16-oxo-17-[2-(piperazin-1-yl)ethyl]-10-{[2-(3,3,3-trifluoropropoxy)pyrimidin-4-yl]methoxy}-7,8,16,17-tetrahydro-15H-18,21-etheno-13,9-(metheno)-6,14-dioxa-2-thia-3,5,17-triazacyclononadeca[1,2,3-cd]indene-7-carboxylate

Example 16M (90 mg) was dissolved in dichloromethane (0.7 mL). Trifluoroacetic acid (0.7 mL) was added. The reaction mixture was stirred at ambient temperature for 10 minutes. LC/MS showed complete conversion to one peak consistent with the desired product. The mixture was concentrated under reduced pressure. The crude product was used in the next step without further purification. LC/MS (APCI) m/z 950.2 (M+H)⁺.

### Example 16O

### ethyl (7R,21S)-19-chloro-1-(4-fluorophenyl)-20-methyl-17-[2-(4-methylpiperazin-1-yl)ethyl]-16-oxo-10-{[2-(3,3,3-trifluoropropoxy)pyrimidin-4-yl]methoxy}-7,8,16,17-tetrahydro-15H-18,21-etheno-13,9-(metheno)-6,14-dioxa-2-thia-3,5,17-triazacyclononadeca[1,2,3-cd]indene-7-carboxylate

Example 16N (69 mg) was dissolved in tetrahydrofuran (1 mL), and formaldehyde (18 mg) followed by sodium triacetoxyborohydride (46 mg) were added. The reaction mixture was stirred at ambient temperature for 1 hour. The reaction mixture was diluted with ethyl acetate and was washed with sodium bicarbonate mixture (0.1 M in water). The organics were dried over anhydrous sodium sulfate, filtered, and concentrated under reduced pressure. The crude product was used in the next step without further purification. LC/MS (APCI) m/z 964.3 (M+H)⁺.

### Example 16P

### (7R,21S)-19-chloro-1-(4-fluorophenyl)-20-methyl-17-[2-(4-methylpiperazin-1-yl)ethyl]-16-oxo-10-{[2-(3,3,3-trifluoropropoxy)pyrimidin-4-yl]methoxy}-7,8,16,17-tetrahydro-15H-18,21-etheno-13,9-(metheno)-6,14-dioxa-2-thia-3,5,17-triazacyclononadeca[1,2,3-cd]indene-7-carboxylic acid

To a mixture of Example 16O (70.4 mg) in tetrahydrofuran (0.50 mL) and methanol (0.50 mL) was added lithium hydroxide mixture (1.0 M in water) (1.10 mL). The mixture was stirred at ambient temperature for 1 hour. The mixture was concentrated, dissolved in N,N-dimethylformamide (1 mL), and acidified with trifluoroacetic acid. The mixture was purified on a Gilson reverse-phase HPLC (Zorbax, C-18, 250×21.2 mm column, 5 to 90% acetonitrile in water (0.1% trifluoroacetic acid)) to provide the title compound. ¹H NMR (500 MHz, dimethyl sulfoxide-d₆) δ ppm 8.76 (s, 1H), 8.57 (d, 1H), 7.28 (d, 1H), 7.20 (d, 1H), 7.16-7.07 (m, 4H), 6.99 (d, 1H), 6.75 (d, 1H), 6.56 (dd, 1H), 6.10 (t, 1H), 6.02 (d, 1H), 5.11-4.98 (m, 2H), 4.83 (d, 1H), 4.57 (d, 1H), 4.53 (t, 2H), 4.42-4.28 (m, 1H), 3.50 (dd, 1H), 3.42-3.27 (m, 2H), 3.25-3.09 (m, 2H), 3.10-2.90 (m, 4H), 2.90-2.80 (m, 2H), 2.78 (s, 3H), 2.43-2.23 (m, 4H), 2.08 (s, 3H). MS (ESI) m/z 936.2 (M+H)⁺.

### Example 17

### (7R,21S)-19-chloro-1-(4-fluorophenyl)-20-methyl-17-[2-(4-methylpiperazin-1-yl)ethyl]-10-{[2-(3,3,3-trifluoropropoxy)pyrimidin-4-yl]methoxy}-7,8,16,17-tetrahydro-15H-18,21-etheno-13,9-(metheno)-6,14-dioxa-2-thia-3,5,17-triazacyclononadeca[1,2,3-cd]indene-7-carboxylic acid

### Example 17A

### 4-bromo-2-chloro-N-(2-chloroethyl)-3-methylaniline

To a stirring mixture of Example 7G (1.00 g) and chloroacetaldehyde (0.691 mL) in 0.78 mL of 1:1 of 6M HCl:methanol in methanol (10 mL) was added sodium cyanoborohydride (314 mg). The reaction mixture was stirred at ambient temperature for 1 day and was concentrated. The mixture was diluted with dichloromethane, washed with sodium bicarbonate mixture (1M in water), dried over anhydrous sodium sulfate, filtered, and concentrated. The residue was purified by silica gel chromatography on an AnaLogix IntelliFlash²⁸⁰ system (0-30% ethyl acetate/heptanes, linear gradient) to provide the title compound. LC/MS (APCI) m/z 283.6 (M+H)⁺.

### Example 17B

### 2-chloro-N-(2-chloroethyl)-3-methyl-4-(4,4,5,5-tetramethyl-1,3,2-dioxaborolan-2-yl)aniline

To a 100 mL flask was added potassium acetate (1.040 g). The flask was capped with septa and heated to 100° C. under high vacuum for 1 hour. After cooling to ambient temperature, bis(pinacolato)diboron (1.795 g), Example 17A (1.00 g), 2-(dicyclohexylphosphino)-2′,4′,6′-triisopropylbiphenyl (50.5 mg) and chloro(2-dicyclohexylphosphino-2′,4′,6′-triisopropyl-1,1′-biphenyl)[2-(2′-amino-1,1′-biphenyl)]palladium(II) (83 mg) were quickly added. The flask was capped and evacuated and backfilled with nitrogen three times. Freshly degassed 2-methyltetrahydrofuran (35 mL) (nitrogen was bubbled through the solvent for 30 minutes prior addition) was introduced via syringe. The stirring mixture was evacuated and backfilled with nitrogen twice again. The mixture was stirred at 65° C. for 30 hours. After cooling to ambient temperature, the mixture was filtered through a bed of diatomaceous earth and was washed with 100 mL of ethyl acetate. The filtrate was concentrated and was purified by silica gel chromatography on an AnaLogix IntelliFlash²⁸⁰ system (0-30% ethyl acetate in heptanes, linear gradient) to provide the title compound. LC/MS (APCI) m/z 329.8 (M+H)⁺.

### Example 17C

### (2R)-ethyl 2-((5-((1S)-3-chloro-4-((2-chloroethyl)amino)-2-methylphenyl)-6-(4-fluorophenyl)thieno[2,3-d]pyrimidin-4-yl)oxy)-3-(5-hydroxy-2-((2-(3,3,3-trifluoropropoxy)pyrimidin-4-yl)methoxy)phenyl)propanoate

Example 16H (700 mg), Example 17B (407 mg), bis(di-tert-butyl(4-dimethylaminophenyl)phosphine)dichloropalladium(II) (67.2 mg) and cesium carbonate (928 mg) were placed in a 5 mL vial, degassed and purged with nitrogen. To the mixture, tetrahydrofuran (6.0 mL) and water (1.5 mL) were added via syringe, and the reaction vessel was degassed and purged with nitrogen. The reaction mixture was heated to 55° C. for 1 hour. The mixture was filtered through diatomaceous earth and washed with ethyl acetate. The organics were concentrated and purified by silica gel chromatography on an AnaLogix IntelliFlash²⁸⁰ system (5-60% ethyl acetate in hexanes, linear gradient) to provide the title compound. LC/MS (APCI) m/z 860.1 (M+H)⁺.

### Example 17D

### ethyl (7R,21S)-19-chloro-1-(4-fluorophenyl)-20-methyl-10-{[2-(3,3,3-trifluoropropoxy)pyrimidin-4-yl]methoxy}-7,8,16,17-tetrahydro-15H-18,21-etheno-13,9-(metheno)-6,14-dioxa-2-thia-3,5,17-triazacyclononadeca[1,2,3-cd]indene-7-carboxylate

A mixture of Example 17C (550 mg), sodium iodide (96 mg) and cesium carbonate (416 mg) in N,N-dimethylformamide (55 mL) was stirred at 45° C. for 18 hours. To the mixture was added water, and the mixture was extracted with ethyl acetate. The organics were washed with brine, dried over anhydrous sodium sulfate, filtered, and concentrated under reduced pressure. The residue was purified by silica gel flash chromatography on an AnaLogix IntelliFlash²⁸⁰ system (0-40% ethyl acetate/heptanes, linear gradient) to provide the title compound. LC/MS (APCI) m/z 824.1 (M+H)⁺.

### Example 17E

### ethyl (7R,21S)-19-chloro-17-(2-chloroethyl)-1-(4-fluorophenyl)-20-methyl-10-{[2-(3,3,3-trifluoropropoxy)pyrimidin-4-yl]methoxy}-7,8,16,17-tetrahydro-15H-18,21-etheno-13,9-(metheno)-6,14-dioxa-2-thia-3,5,17-triazacyclononadeca[1,2,3-cd]indene-7-carboxylate

To a stirring mixture of Example 17D (115 mg) and chloroacetaldehyde (0.035 mL) in 0.1 mL of 1:1 of 6M HCl:methanol in methanol (1 mL) was added sodium cyanoborohydride (17.54 mg). The reaction mixture was stirred at ambient temperature for 1 day. The mixture was diluted with ethyl acetate, washed with sodium bicarbonate mixture (1M in water), dried over anhydrous sodium sulfate, filtered, and concentrated. The residue was purified by silica gel chromatography on an AnaLogix IntelliFlash²⁸⁰ system (5-60% ethyl acetate in hexanes, linear gradient) to provide the title compound. LC/MS (APCI) m/z 886.1 (M+H)⁺.

### Example 17F

### ethyl (7R,21S)-19-chloro-1-(4-fluorophenyl)-20-methyl-17-[2-(4-methylpiperazin-1-yl)ethyl]-10-{[2-(3,3,3-trifluoropropoxy)pyrimidin-4-yl]methoxy}-7,8,16,17-tetrahydro-15H-18,21-etheno-13,9-(metheno)-6,14-dioxa-2-thia-3,5,17-triazacyclononadeca[1,2,3-cd]indene-7-carboxylate

To a stirring mixture of Example 17E (58 mg) in propiononitrile (0.5 mL) were added 1-methylpiperazine (10.48 mg), sodium iodide (15.69 mg) and sodium carbonate (11.09 mg). The reaction mixture was stirred at 75° C. overnight. The mixture was filtered through diatomaceous earth, rinsed with ethanol/methanol (10/1), and concentrated under reduced pressure. The crude product was used in the next step without further purification. LC/MS (APCI) m/z 950.2 (M+H)⁺.

### Example 17G

### (7R,21S)-19-chloro-1-(4-fluorophenyl)-20-methyl-17-[2-(4-methylpiperazin-1-yl)ethyl]-10-{[2-(3,3,3-trifluoropropoxy)pyrimidin-4-yl]methoxy}-7,8,16,17-tetrahydro-15H-18,21-etheno-13,9-(metheno)-6,14-dioxa-2-thia-3,5,17-triazacyclononadeca[1,2,3-cd]indene-7-carboxylic acid

To a mixture of Example 17F (38.0 mg) in tetrahydrofuran (0.40 mL) and methanol (0.40 mL) was added lithium hydroxide (0.60 mL, 1.0 M in water). The mixture was stirred at ambient temperature for 6 hours. The mixture was concentrated, dissolved in N,N-dimethylformamide (1 mL), and acidified with trifluoroacetic acid. The mixture was purified on a Gilson prep HPLC (Zorbax, C-18, 250×21.2 mm column, 5 to 90% acetonitrile in water (0.1% trifluoroacetic acid)) to provide the title compound. ¹H NMR (400 MHz, dimethyl sulfoxide-d₆) δ ppm 8.73 (s, 1H), 8.62 (d, 2H), 7.27 (d, 1H), 7.24-7.11 (m, 5H), 6.91 (d, 1H), 6.82 (d, 1H), 6.74 (dd, 1H), 6.13 (dd, 1H), 5.65 (d, 1H), 5.06 (d2H), 4.53 (t, 2H), 4.40 (dd, 1H), 4.08-3.91 (m, 1H), 3.81 (dd, 1H), 3.67-3.55 (m, 3H), 3.31-3.15 (m, 5H), 2.93-2.78 (m, 5H), 2.76 (s, 3H), 2.65 (d, 3H), 2.20 (s, 3H). MS (ESI) m/z 922.2 (M+H)⁺.

### Example 18

### (7R,21S)-19-chloro-1-(4-fluorophenyl)-20-methyl-10-{[2-(3,3,3-trifluoropropoxy)pyrimidin-4-yl]methoxy}-7,8,16,17-tetrahydro-15H-18,21-etheno-13,9-(metheno)-6,14-dioxa-2-thia-3,5,17-triazacyclononadeca[1,2,3-cd]indene-7-carboxylic acid

To a mixture of Example 17D (34 mg) in tetrahydrofuran (0.50 mL) and methanol (0.50 mL) was added lithium hydroxide (0.619 mL, 1.0 M in water). The mixture was stirred at ambient temperature for 1 day and was concentrated. The residue was dissolved in N,N-dimethylformamide (1 mL) and was acidified with trifluoroacetic acid. The mixture was purified on a Gilson prep HPLC (Zorbax, C-18, 250×21.2 mm column, 5 to 90% acetonitrile in water (0.1% trifluoroacetic acid)) to provide the title compound after lyophilization. ¹H NMR (400 MHz, dimethyl sulfoxide-d₆) δ ppm 12.76 (s, 1H), 8.67 (s, 1H), 8.60 (d, 1H), 7.35-7.27 (m, 2H), 7.25 (d, 1H), 7.23-7.16 (m, 2H), 6.95-6.67 (m, 4H), 5.99 (dd, 1H), 5.84 (d, 1H), 5.25 (s, 1H), 5.01 (s, 2H), 4.52 (t, 2H), 4.42-4.27 (m, 1H), 3.97-3.81 (m, 2H), 3.76 (dd, 1H), 3.24-3.13 (m, 1H), 2.89-2.66 (m, 3H), 2.09 (s, 3H). MS (ESI) m/z 796.1 (M+H)⁺.

### Example 19

### (7R,21R)-19-chloro-1-(4-fluorophenyl)-20-methyl-10-{[2-(3,3,3-trifluoropropoxy)pyrimidin-4-yl]methoxy}-7,8,16,17-tetrahydro-15H-18,21-etheno-13,9-(metheno)-6,14-dioxa-2-thia-3,5,17-triazacyclononadeca[1,2,3-cd]indene-7-carboxylic acid

The title compound was isolated during the synthesis of Example 18. ¹H NMR (400 MHz, dimethyl sulfoxide-d₆) δ ppm 13.23 (s, 1H), 8.61-8.53 (m, 2H), 7.41 (d, 1H), 7.36-7.31 (m, 2H), 7.24-7.12 (m, 2H), 6.81-6.69 (m, 2H), 6.63 (d, 1H), 6.43 (d, 1H), 6.12 (d, 1H), 5.94 (s, 1H), 5.72 (dd, 1H), 5.08 (q, 2H), 4.57-4.43 (m, 2H), 4.29-4.15 (m, 1H), 3.90 (ddd, 1H), 3.78 (d, 1H), 3.53-3.44 (m, 2H), 2.79 (qt, 2H), 2.46-2.39 (m, 1H), 2.38 (s, 3H). MS (ESI) m/z 796.0 (M+H)⁺.

### Example 20

### (7R,20S)-18-chloro-1-(4-fluorophenyl)-10-{[2-(2-methoxyphenyl)pyrimidin-4-yl]methoxy}-19-methyl-15-[2-(morpholin-4-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylic acid

### Example 20A

### (R)-ethyl 2-acetoxy-3-(5-cyano-2-((2-(2-methoxyphenyl)pyrimidin-4-yl)methoxy)phenyl)propanoate

A mixture of Example 1L (3 g), zinc cyanide (0.799 g) and tetrakis(triphenylphosphine)palladium (0) (0.65 g) in anhydrous N,N-dimethylformamide (20 mL) was purged with nitrogen and stirred at 70° C. overnight. The reaction mixture was quenched with water, extracted three times with ethyl acetate (100 mL), dried over magnesium sulfate, filtered and concentrated. The residue was purified by silica gel chromatography (60% ethyl acetate in hexane) to provide the title compound. MS (DCI) m/z 476 (M+H)⁺.

### Example 20B

### (R)-ethyl 2-acetoxy-3-(5-formyl-2-((2-(2-methoxyphenyl)pyrimidin-4-yl)methoxy)phenyl)propanoate

A mixture of Example 20A (0.5 g) in 60% of acetic acid in water (25 mL) was treated with Raney Nickel (100 mg). The mixture was stirred at room temperature under hydrogen overnight. The reaction mixture was filtered, and the filtrate was concentrated. The residue was purified by silica gel chromatography (60% ethyl acetate in hexane) to provide the title compound. MS (DCI) m/z 479 (M+H)⁺.

### Example 20C

### (R)-ethyl 2-acetoxy-3-(5-(((tert-butoxycarbonyl)(2-morpholinoethyl)amino)methyl)-2-((2-(2-methoxyphenyl)pyrimidin-4-yl)methoxy)phenyl)propanoate

To a mixture of Example 20B (300 mg) in dichloromethane (5 mL) was added 2-morpholinoethanamine (98 mg). The mixture was stirred at room temperature for 1 hour before the addition of sodium triacetoxyborohydride (199 mg). The mixture was stirred at room temperature for 4 hours and quenched by the addition of saturated aqueous sodium bicarbonate mixture. The reaction mixture was partitioned between ethyl acetate (100 mL) and brine (100 mL). The organic phase was concentrated and dissolved in tetrahydrofuran (5 mL). To the mixture was added di-tert-butyldicarbonate (151 mg) and 4-dimethylaminopyridine (0.8 mg). The mixture was stirred at room temperature for 30 min. The reaction mixture was concentrated and was purified by silica gel chromatography (60% ethyl acetate in hexane) to provide the title compound. MS (DCI) m/z 693 (M+H)⁺.

### Example 20D

### ethyl (R)-3-(5-(((tert-butoxycarbonyl)(2-morpholinoethyl)amino)methyl)-2-((2-(2-methoxyphenyl)pyrimidin-4-yl)methoxy)phenyl)-2-hydroxypropanoate

Example 20D was prepared according to the procedure described for Example 10, substituting Example 20C for Example 1N.

### Example 20E

### (R)-ethyl 2-((5-bromo-6-(4-fluorophenyl)thieno[2,3-d]pyrimidin-4-yl)oxy)-3-(5-(((tert-butoxycarbonyl)(2-morpholinoethyl)amino)methyl)-2-((2-(2-methoxyphenyl)pyrimidin-4-yl)methoxy)phenyl)propanoate

To a flask containing Example 20D (300 mg), cesium carbonate (300 mg) and anhydrous tert-butanol (5 mL) was added Example 1D (170 mg). The mixture was stirred at 65° C. overnight. The reaction mixture was diluted with dichloromethane (100 mL), and the material was filtered. The organic phase was concentrated and was purified by silica gel chromatography (20% methanol in ethyl acetate) to provide the title compound. MS (DCI) m/z 958 (M+H)⁺.

### Example 20F

### (4-bromo-2-chloro-3-methylphenyl)methanol

To a cold (0° C. external bath) mixture of Example 10A (20 g) in methanol (200 mL) was added sodium borohydride (4.86 g), portionwise. The reaction warmed to room temperature overnight and was quenched by the addition of 1 M aqueous HCl (150 mL), water (100 mL) and ethyl acetate (200 mL). The layers were separated, and the aqueous layer was extracted with additional ethyl acetate (100 mL×2). The combined organic layers were washed with water and brine, dried over anhydrous magnesium sulfate, filtered and concentrated under reduced pressure to provide the title compound, which was used in the subsequent step without further purification. ¹H NMR (500 MHz, chloroform-d) δ ppm 7.5 (d, 1H), 7.2 (d, 1H), 4.75 (d, 1H), and 2.55 (s, 3H).

### Example 20G

### ((4-bromo-2-chloro-3-methylbenzyl)oxy)(tert-butyl)dimethylsilane

To a mixture of Example 20F (170 mg) and 1H-imidazole (74 mg) in N,N-dimethylformamide (5 mL) was added tert-butylchlorodimethylsilane (163 mg). The reaction mixture was stirred for 1 hour at room temperature. Ethyl acetate (50 mL) and water (30 mL) were added, and the layers were separated. The organic phase was washed with brine and concentrated. The residue was purified by silica gel column chromatography (5% ethyl acetate in heptane) to provide the title compound. MS (DCI) m/z 350 (M+H)⁺.

### Example 20H

### tert-butyl((2-chloro-3-methyl-4-(4,4,5,5-tetramethyl-1,3,2-dioxaborolan-2-yl)benzyl)oxy)dimethylsilane

A mixture of Example 20G (1.1 g) in tetrahydrofuran (10 mL) was cooled to −78° C., n-butyllithium (2.4 mL, 2.5 M in hexane) was added to the reaction, and the reaction mixture was stirred at −78° C. for 30 minutes. 2-Isopropoxy-4,4,5,5-tetramethyl-1,3,2-dioxaborolane (696 mg) was added to the mixture, and the mixture was warmed to room temperature. The reaction mixture was partitioned between ethyl acetate (100 mL) and brine (100 mL). The organic phase was concentrated and purified by silica gel column chromatography (10% ethyl acetate in heptane) to provide the title compound. MS (DCI) m/z 397 (M+H)⁺.

### Example 20I

### (2R)-ethyl 3-(5-(((tert-butoxycarbonyl)(2-morpholinoethyl)amino)methyl)-2-((2-(2-methoxyphenyl)pyrimidin-4-yl)methoxy)phenyl)-2-((5-((1S)-4-(((tert-butyldimethylsilyl)oxy)methyl)-3-chloro-2-methylphenyl)-6-(4-fluorophenyl)thieno[2,3-d]pyrimidin-4-yl)oxy)propanoate

A mixture of Example 20E (130 mg), Example 20H (81 mg), bis(di-tert-butyl(4-dimethylaminophenyl)phosphine)dichloropalladium(II) (10 mg) and cesium carbonate (88 mg) was evacuated and filled with argon. To the mixture a degassed mixture of tetrahydrofuran (6 mL) and water (1.8 mL) was added. The reaction mixture was stirred at 40° C. overnight. The reaction mixture was concentrated and was purified by silica gel chromatography (eluting with a gradient of ethyl acetate in heptane of 60-100%) to provide the title compound. MS (DCI) m/z 1148 (M+H)⁺.

### Example 20J

### (2R)-ethyl 3-(5-(((tert-butoxycarbonyl)(2-morpholinoethyl)amino)methyl)-2-((2-(2-methoxyphenyl)pyrimidin-4-yl)methoxy)phenyl)-2-((5-((1S)-3-chloro-4-formyl-2-methylphenyl)-6-(4-fluorophenyl)thieno[2,3-d]pyrimidin-4-yl)oxy)propanoate

A mixture of Example 20I (110 mg) in tetrahydrofuran (5 mL) was cooled to 0° C., and tetrabutylammonium fluoride (0.2 mL, 1M in tetrahydrofuran) was added. The reaction mixture was stirred at 0° C. for 1 hour. The reaction mixture was quenched with water and was extracted with ethyl acetate (2×100 mL). The organic phase was concentrated and was redissolved in dichloromethane (5 mL). To the mixture, Dess-Martin periodinane (41 mg) in dichloromethane (1 mL) was added. The reaction mixture was stirred at room temperature for about 30 minutes. The reaction mixture was concentrated and was purified by silica gel chromatography (eluting with 100% ethyl acetate) to provide the title compound. MS (DCI) m/z 1032 (M+H)⁺.

### Example 20K

### (7R,20S)-18-chloro-1-(4-fluorophenyl)-10-{[2-(2-methoxyphenyl)pyrimidin-4-yl]methoxy}-19-methyl-15-[2-(morpholin-4-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylic acid

To Example 20J (80 mg) in dichloromethane (2 mL) was added trifluoroacetic acid (0.5 mL). The mixture was stirred at room temperature for 3 hours. The mixture was concentrated and partitioned between ethyl acetate (100 mL) and sodium bicarbonate mixture (30 mL). The organic phase was dried with magnesium sulfate, filtered, and concentrated. The intermediate was dissolved in dichloromethane (5 mL), and magnesium sulfate (500 mg) was added. The mixture was stirred at room temperature for 1 hour before sodium triacetoxyborohydride (46 mg) was added. The mixture was stirred for another 20 minutes and was concentrated under vacuum. The reaction mixture was partitioned between ethyl acetate (100 mL) and brine. The organic phase was dried with magnesium sulfate, filtered and concentrated. The crude product was dissolved in a mixed solvent of tetrahydrofuran (4 mL), water (2 mL), and methanol (2 mL). Lithium hydroxide monohydrate (8 mg) was added. The reaction mixture was stirred at room temperature for two days. The mixture was acidified by adding trifluoroacetic acid and was concentrated. The residue was purified by reverse phase HPLC (Zorbax C-18, 10 to 50% acetonitrile in water containing 0.1% v/v trifluoroacetic acid) to provide the title compound as a trifluoroacetic acid salt. ¹H NMR (501 MHz, dimethyl sulfoxide-d₆) δ ppm 8.63 (s, 1H), 8.61 (d, 1H), 7.49 (dd, 1H), 7.45 (ddd, 1H), 7.40 (d, 1H), 7.27-7.16 (m, 5H), 7.13 (ddd, 3H), 7.03 (td, 2H), 6.73 (d, 1H), 6.35 (d, 1H), 5.91 (dd, 1H), 5.20-4.97 (m, 2H), 4.00-3.56 (m, 5H), 3.74 (s, 3H), 3.44 (t, 2H), 3.32 (t, 4H), 3.19 (dtd, 3H), 2.48 (p, 4H), 1.74 (s, 3H). MS (ESI) m/z 888 (M+H)⁺.

### Example 21

### [(2,2-dimethylpropanoyl)oxy]methyl (7R,21S)-19-chloro-1-(4-fluorophenyl)-10-{[2-(2-methoxyphenyl)pyrimidin-4-yl]methoxy}-20-methyl-16-[2-(4-methylpiperazin-1-yl)ethyl]-15-oxo-7,8,14,15,16,17-hexahydro-18,21-etheno-13,9-(metheno)-6-oxa-2-thia-3,5,16-triazacyclononadeca[1,2,3-cd]indene-7-carboxylate

Example 11F (120 mg), sodium iodide (29.6 mg) and cesium carbonate (300 mg) were added to N,N-dimethylformamide (0.8 mL) and chloromethyl pivalate (35 mg) was added. The mixture was stirred at ambient temperature overnight. Water (2.5 mL) was added, and the precipitate was extracted with three portions of ethyl acetate. The organic layers were combined, dried over anhydrous magnesium sulfate, filtered and concentrated. The residue was purified by silica gel preparative thin-layer chromatography (20×20 cm; 1 mm thick; eluting 40% of 2:1 methanol:water in ethyl acetate) to provide the title compound. ¹H NMR (500 MHz, dimethyl sulfoxide-d₆) δ ppm 1.03 (s, 9H), 1.23 (s, 3H), 1.83 (s, 3H), 2.13 (s, 3H), 2.22-2.44 (m, 3H), 2.45-2.50 (m, 1H), 2.55-2.64 (m, 1H), 3.04-3.58 (m, 8H), 3.74 (s, 3H), 3.82 (d, 1H), 3.93-4.03 (m, 1H), 4.48 (d, 1H), 4.87 (d, 1H), 4.93 (d, 1H), 5.73-5.79 (m, 2H), 6.46-6.67 (m, 1H), 6.79 (d, 1H), 7.03-7.11 (m, 3H), 7.12-7.21 (m, 4H), 7.22-7.31 (m, 3H), 7.44-7.50 (m, 1H), 7.50-7.54 (m, 1H), 8.47 (s, 1H), 8.74 (d, 1H). LC/MS (APCI) m/z 1042.5 (M+H)⁺.

### Example 22

### (7R,20S)-18-chloro-1-(4-fluorophenyl)-15-(2-methoxyethyl)-10-{[2-(2-methoxyphenyl)pyrimidin-4-yl]methoxy}-19-methyl-7,8,15,16-tetrahydro-14H-17,20-etheno-13,9-(metheno)-6-oxa-2-thia-3,5,15-triazacyclooctadeca[1,2,3-cd]indene-7-carboxylic acid

### Example 22A

### ethyl (R)-2-acetoxy-3-(5-(((tert-butoxycarbonyl)(2-methoxyethyl)amino)methyl)-2-((2-(2-methoxyphenyl)pyrimidin-4-yl)methoxy)phenyl)propanoate

The title compound was prepared as described in Example 20C by replacing 2-morpholinoethanamine with 2-methoxyethanamine. MS (ESI) m/z 638 (M+H)⁺.

### Example 22B

### (R)-ethyl 3-(5-(((tert-butoxycarbonyl)(2-methoxyethyl)amino)methyl)-2-((2-(2-methoxyphenyl)pyrimidin-4-yl)methoxy)phenyl)-2-hydroxypropanoate

The title compound was prepared as described in Example 10 by replacing Example 1N with Example 22A. MS (ESI) m/z 596 (M+H)⁺.

### Example 22C

### (R)-ethyl 2-((5-bromo-6-(4-fluorophenyl)thieno[2,3-d]pyrimidin-4-yl)oxy)-3-(5-(((tert-butoxycarbonyl)(2-morpholinoethyl)amino)methyl)-2-((2-(2-methoxyphenyl)pyrimidin-4-yl)methoxy)phenyl)propanoate

The title compound was prepared as described in Example 20E by replacing Example 20D with Example 22B. MS (ESI) m/z 902 (M+H)⁺.

### Example 22D

### (2R)-ethyl 3-(5-(((tert-butoxycarbonyl)(2-methoxyethyl)amino)methyl)-2-((2-(2-methoxyphenyl)pyrimidin-4-yl)methoxy)phenyl)-2-((5-((1S)-4-(((tert-butyldimethylsilyl)oxy)methyl)-3-chloro-2-methylphenyl)-6-(4-fluorophenyl)thieno[2,3-d]pyrimidin-4-yl)oxy)propanoate

The title compound was prepared as described in Example 20I by replacing Example 20E with Example 22C. MS (ESI) m/z 1093 (M+H)⁺.

### Example 22E

### (2R)-ethyl 3-(5-(((tert-butoxycarbonyl)(2-methoxyethyl)amino)methyl)-2-((2-(2-methoxyphenyl)pyrimidin-4-yl)methoxy)phenyl)-2-((5-((1S)-3-chloro-4-formyl-2-methylphenyl)-6-(4-fluorophenyl)thieno[2,3-d]pyrimidin-4-yl)oxy)propanoate

The title compound was prepared as described in Example 20J by replacing Example 20I with Example 22D. MS (ESI) m/z 977 (M+H)⁺.

### Example 22F

### (7R,20S)-18-chloro-1-(4-fluorophenyl)-15-(2-methoxyethyl)-10-{[2-(2-methoxyphenyl)pyrimidin-4-yl]methoxy}-19-methyl-7,8,15,16-tetrahydro-14H-17,20-etheno-13,9-(metheno)-6-oxa-2-thia-3,5,15-triazacyclooctadeca[1,2,3-cd]indene-7-carboxylic acid

The title compound was prepared as described in Example 20K by replacing Example 20J with Example 22E. ¹H NMR (400 MHz, dimethyl sulfoxide-d₆) δ ppm 10.35 (s, 1H), 8.67-8.61 (m, 1H), 7.61-7.56 (m, 1H), 7.50 (dd, J=7.6, 1.8 Hz, 1H), 7.50-7.38 (m, 1H), 7.38-7.08 (m, 10H), 7.03 (td, J=7.5, 1.0 Hz, 1H), 6.90 (d, J=8.5 Hz, 1H), 6.58-6.53 (m, 1H), 5.98 (m, 1H), 5.29-5.16 (m, 1H), 5.08 (d, J=14.9 Hz, 1H), 4.63-4.48 (m, 1H), 4.37 (m, 1H), 4.29 (d, J=13.8 Hz, 1H), 3.92 (q, J=4.6, 4.2 Hz, 2H), 3.74 (s, 3H), 3.37 (s, 3H), 3.23 (d, J=13.9 Hz, 3H), 2.96 (d, J=6.7 Hz, 1H), 1.73 (s, 3H). MS (ESI) m/z 833 (M+H)⁺.

### Example 23

### (7R,20S)-18-chloro-15-[2-(4,4-difluoropiperidin-1-yl)ethyl]-1-(4-fluorophenyl)-10-{[2-(2-methoxyphenyl)pyrimidin-4-yl]methoxy}-19-methyl-7,8,15,16-tetrahydro-14H-17,20-etheno-13,9-(metheno)-6-oxa-2-thia-3,5,15-triazacyclooctadeca[1,2,3-cd]indene-7-carboxylic acid

To a mixture of Example 1U (100 mg) in dichloromethane (5 mL) and acetic acid (1 mL) was added 2-(4,4-difluoropiperidin-1-yl)ethanamine (39 mg). The mixture was stirred at room temperature for 1 hour before the addition of sodium triacetoxyborohydride (186 mg). The mixture was stirred at room temperature for 1 hour and was quenched by the addition of saturated aqueous sodium bicarbonate mixture. The reaction mixture was extracted with ethyl acetate (50 mL×2). The combined organic layers were washed with brine and dried over sodium sulfate. The mixture was filtered, and the solvents were removed under reduced pressure. The residue was dissolved in a mixture of trifluoroacetic acid/tetrahydrofuran/water (3/3/0.5). The reaction mixture was stirred at room temperature for 1 hour and was quenched by the addition of saturated aqueous sodium bicarbonate mixture. The reaction mixture was extracted with ethyl acetate (50 mL×2). The combined extracts were washed with brine, dried over sodium sulfate, filtered and concentrated under reduced pressure. The residue was dissolved in dichloromethane (5 mL) and magnesium sulfate (500 mg) was added. The mixture was stirred at room temperature for 1 hour before sodium triacetoxyborohydride (210 mg) was added. The mixture was stirred for 20 minutes, and quenched by the addition of ethyl acetate (100 mL) and saturated aqueous sodium bicarbonate mixture (30 mL). The layers were separated, and the organic layer was washed with additional saturated aqueous sodium bicarbonate mixture and brine (30 mL). The organic phase was dried with magnesium sulfate, filtered and concentrated under reduced pressure. The residue was dissolved in a mixed solvent system of tetrahydrofuran (8 mL), water (4 mL), and methanol (4 mL), and solid lithium hydroxide monohydrate (10 mg) was added. The reaction mixture was stirred at room temperature for 3 hours, and the mixture was acidified with trifluoroacetic acid (0.1 mL) and was concentrated under reduced pressure. The residue was dissolved in dimethylsulfoxide/methanol and was purified by reverse-phase HPLC (Zorbax C-18, 10 to 80% acetonitrile in water containing 0.1% v/v trifluoracetic acid) to provide the title compound. ¹H NMR (400 MHz, dimethyl sulfoxide-d₆) δ ppm 8.64-8.55 (m, 2H), 7.53-7.35 (m, 4H), 7.24-7.16 (m, 4H), 7.12 (ddd, 3H), 7.08-6.97 (m, 2H), 6.74 (d, 1H), 6.33 (d, 1H), 5.90 (dd, 1H), 5.18-4.96 (m, 2H), 4.03-3.74 (m, 5H), 3.72 (s, 3H), 3.43 (dt, 3H), 3.35-3.05 (m, 2H), 2.47 (p, 4H), 2.28 (dp, 4H), 1.72 (s, 3H). MS (ESI) m/z 922 (M+H)⁺.

### Example 24

### (7R,20S)-18-chloro-1-(4-fluorophenyl)-15-[2-(2-methoxyethoxy)ethyl]-10-{[2-(2-methoxyphenyl)pyrimidin-4-yl]methoxy}-19-methyl-7,8,15,16-tetrahydro-14H-17,20-etheno-13,9-(metheno)-6-oxa-2-thia-3,5,15-triazacyclooctadeca[1,2,3-cd]indene-7-carboxylic acid

The title compound was prepared according to the procedure described in Example 23, substituting 2-(2-methoxyethoxy)ethanamine for 2-(4,4-difluoropiperidin-1-yl)ethanamine. ¹H NMR (400 MHz, dimethyl sulfoxide-d₆) δ ppm 8.65-8.59 (m, 2H), 7.50-7.38 (m, 5H), 7.31 (dtd, 4H), 7.25-7.07 (m, 2H), 7.00 (qd, 2H), 6.82 (d, 1H), 6.02-5.88 (m, 1H), 5.54-5.43 (m, 1H), 5.24 (d, 1H), 4.60-4.39 (m, 2H), 3.95 (dd, 2H), 3.72 (s, 3H), 3.66-3.55 (m, 4H), 3.53-3.44 (m, 2H), 3.43-3.38 (m, 2H), 3.17 (s, 3H), 3.03-2.85 (m, 2H), 2.71-2.59 (m, 1H), 1.89 (s, 3H). MS (ESI) m/z 877 (M+H)⁺.

### Example 25

### (7R,21S)-19-chloro-1-(4-fluorophenyl)-10-{[2-(2-methoxyphenyl)pyrimidin-4-yl]methoxy}-20-methyl-17-[2-(4-methylpiperazin-1-yl)ethyl]-16-oxo-7,8,14,15,16,17-hexahydro-18,21-etheno-13,9-(metheno)-6-oxa-2-thia-3,5,17-triazacyclononadeca[1,2,3-cd]indene-7-carboxylic acid

### Example 25A

### (R)-ethyl 2-acetoxy-3-(5-(3-(tert-butoxy)-3-oxopropyl)-2-((2-(2-methoxyphenyl)pyrimidin-4-yl)methoxy)phenyl)propanoate

The title compound was prepared as described in Example 11A substituting (3-(tert-butoxy)-3-oxopropyl)zinc(II) bromide (0.5 molar in diethyl ether mixture) for 2-tert-butoxy-2-oxoethylzinc chloride. LC/MS (APCI) m/z 579.3 (M+H)⁺.

### Example 25B

### (R)-ethyl 3-(5-(3-(tert-butoxy)-3-oxopropyl)-2-((2-(2-methoxyphenyl)pyrimidin-4-yl)methoxy)phenyl)-2-hydroxypropanoate

The title compound was prepared as described in Example 7L, substituting Example 25A for Example 7K. LC/MS (APCI) m/z 523.2 (M+H)⁺.

### Example 25C

### (R)-ethyl 2-((5-bromo-6-(4-fluorophenyl)thieno[2,3-d]pyrimidin-4-yl)oxy)-3-(5-(3-(tert-butoxy)-3-oxopropyl)-2-((2-(2-methoxyphenyl)pyrimidin-4-yl)methoxy)phenyl)propanoate

The title compound was prepared as described in Example 7M, substituting Example 25B for Example 7L. LC/MS (APCI) m/z 843.1 (M+H)⁺.

### Example 25D

### (R)-ethyl 2-((5-((1S)-4-amino-3-chloro-2-methylphenyl)-6-(4-fluorophenyl)thieno[2,3-d]pyrimidin-4-yl)oxy)-3-(5-(3-(tert-butoxy)-3-oxopropyl)-2-((2-(2-methoxyphenyl)pyrimidin-4-yl)methoxy)phenyl)propanoate

The title compound was prepared as described in Example 7N, substituting Example 25C for Example 7M. LC/MS (APCI) m/z 904.0 (M+H)⁺.

### Example 25E

### 3-(3-((R)-2-((5-((1S)-4-amino-3-chloro-2-methylphenyl)-6-(4-fluorophenyl)thieno[2,3-d]pyrimidin-4-yl)oxy)-3-ethoxy-3-oxopropyl)-4-((2-(2-methoxyphenyl)pyrimidin-4-yl)methoxy)phenyl)propanoic acid

The title compound was prepared as described in Example 7O substituting Example 25D for Example 7N. LC/MS (APCI) m/z 848.2 (M+H)⁺.

### Example 25F

### ethyl (7R,21S)-19-chloro-1-(4-fluorophenyl)-10-{[2-(2-methoxyphenyl)pyrimidin-4-yl]methoxy}-20-methyl-16-oxo-7,8,14,15,16,17-hexahydro-18,21-etheno-13,9-(metheno)-6-oxa-2-thia-3,5,17-triazacyclononadeca[1,2,3-cd]indene-7-carboxylate

The title compound was prepared as described in Example 7P, substituting Example 25E for Example 7O. LC/MS (APCI) m/z 830.2 (M+H)⁺.

### Example 25G

### ethyl (7R,21S)-17-{2-[4-(tert-butoxycarbonyl)piperazin-1-yl]ethyl}-19-chloro-1-(4-fluorophenyl)-10-{[2-(2-methoxyphenyl)pyrimidin-4-yl]methoxy}-20-methyl-16-oxo-7,8,14,15,16,17-hexahydro-18,21-etheno-13,9-(metheno)-6-oxa-2-thia-3,5,17-triazacyclononadeca[1,2,3-cd]indene-7-carboxylate

The title compound was prepared as described in Example 7Q, substituting Example 25F for Example 7P. LC/MS (APCI) m/z 1042.4 (M+H)⁺.

### Example 25H

### (7R,21S)-19-chloro-1-(4-fluorophenyl)-10-{[2-(2-methoxyphenyl)pyrimidin-4-yl]methoxy}-20-methyl-17-[2-(4-methylpiperazin-1-yl)ethyl]-16-oxo-7,8,14,15,16,17-hexahydro-18,21-etheno-13,9-(metheno)-6-oxa-2-thia-3,5,17-triazacyclononadeca[1,2,3-cd]indene-7-carboxylic acid

The title compound was prepared as described in Example 9, substituting Example 25G for Example 7Q. ¹H NMR (400 MHz, dimethyl sulfoxide-d₆) δ ppm 2.11 (s, 3H), 2.18-2.31 (m, 1H), 2.33-2.45 (m, 1H), 2.57 (t, 2H), 2.63-2.73 (m, 1H), 2.76 (s, 3H), 2.87-3.50 (m, 12H), 3.58 (dd, 1H), 3.72 (s, 3H), 4.02-4.14 (m, 1H), 5.08-5.19 (m, 2H), 5.85-5.97 (m, 1H), 6.25 (d, 1H), 6.79 (d, 1H), 6.89 (dd, 1H), 7.01 (td, J=7.5, 1.0 Hz, 1H), 7.09-7.22 (m, 5H), 7.30 (d, 1H), 7.39-7.47 (m, 2H), 7.47-7.55 (m, 2H), 8.72 (s, 1H), 8.85 (d, 1H). LC/MS (APCI) m/z 928.2 (M+H)⁺.

### Example 26

### (7R,21R)-19-chloro-1-(4-fluorophenyl)-10-{[2-(2-methoxyphenyl)pyrimidin-4-yl]methoxy}-20-methyl-17-[2-(4-methylpiperazin-1-yl)ethyl]-16-oxo-7,8,14,15,16,17-hexahydro-18,21-etheno-13,9-(metheno)-6-oxa-2-thia-3,5,17-triazacyclononadeca[1,2,3-cd]indene-7-carboxylic acid

The title compound was obtained as a side product during the synthesis of Example 25H and was isolated by Gilson reverse-phase prep reverse-phase HPLC (Zorbax, C-18, 250×21.2 mm column, Mobile phase A: 0.1% trifluoroacetic acid in water; B: 0.1% trifluoroacetic acid in acetonitrile; 10-100% B to A gradient). ¹H NMR (400 MHz, dimethyl sulfoxide-d₆) δ ppm 1.89-2.05 (m, 1H), 2.07-2.19 (m, 1H), 2.32-2.60 (m, 8H), 2.63-2.73 (m, 1H), 2.88-3.51 (m, 12H), 3.71 (s, 3H), 4.08 (dd, 1H), 5.10-5.24 (m, 2H), 6.08 (dd, 1H), 6.27 (d, 1H), 6.79-6.87 (m, 1H), 6.88-6.96 (m, 2H), 6.96-7.03 (m, 1H), 7.07-7.23 (m, 5H), 7.26 (d, 1H), 7.37-7.44 (m, 1H), 7.45-7.50 (m, 1H), 7.53 (d, 1H), 8.72 (s, 1H), 8.84 (d, 1H). LC/MS (APCI) m/z 928.2 (M+H)⁺.

### Example 27

### (5-methyl-2-oxo-2H-1,3-dioxol-4-yl)methyl (7S,21S)-19-chloro-1-(4-fluorophenyl)-10-{[2-(2-methoxyphenyl)pyrimidin-4-yl]methoxy}-20-methyl-16-[2-(4-methylpiperazin-1-yl)ethyl]-15-oxo-7,8,14,15,16,17-hexahydro-18,21-etheno-13,9-(metheno)-6-oxa-2-thia-3,5,16-triazacyclononadeca[1,2,3-cd]indene-7-carboxylate

The title compound was prepared as described in Example 21, substituting 4-chloromethyl-5-methyl-1,3-dioxol-2-one for chloromethyl pivalate. ¹H NMR (400 MHz, dimethyl sulfoxide-d₆) δ ppm 1.88 (s, 3H), 2.06 (s, 3H), 2.83 (s, 3H), 2.97-3.57 (m, 15H), 3.71 (s, 3H), 3.76 (d, 1H), 4.29-4.39 (m, 1H), 4.49 (d, 1H), 4.75-4.92 (m, 2H), 4.93-5.04 (m, 2H), 6.47-6.66 (m, 1H), 6.76 (d, 1H), 6.97-7.30 (m, 10H), 7.40-7.54 (m, 2H), 8.39 (s, 1H), 8.70 (d, 1H). LC/MS (APCI) m/z 1040.3 (M+H)⁺.

### Example 28

### (5-methyl-2-oxo-2H-1,3-dioxol-4-yl)methyl (7R,21S)-19-chloro-1-(4-fluorophenyl)-10-{[2-(2-methoxyphenyl)pyrimidin-4-yl]methoxy}-20-methyl-16-[2-(4-methylpiperazin-1-yl)ethyl]-15-oxo-7,8,14,15,16,17-hexahydro-18,21-etheno-13,9-(metheno)-6-oxa-2-thia-3,5,16-triazacyclononadeca[1,2,3-cd]indene-7-carboxylate

The title compound was isolated during the synthesis of Example 27. ¹H NMR (400 MHz, dimethyl sulfoxide-d₆) δ ppm 1.88 (s, 3H), 2.06 (s, 3H), 2.20 (s, 3H), 2.95-3.50 (m, 10H), 3.54-3.66 (m, 5H), 3.71 (s, 3H), 4.21-4.34 (m, 1H), 4.46 (d, 1H), 4.72 (s, 2H), 4.77-4.90 (m, 2H), 4.91-5.05 (m, 2H), 6.44-6.59 (m, 1H), 6.76 (d, 1H), 6.98-7.29 (m, 10H), 7.40-7.52 (m, 2H), 8.39 (s, 1H), 8.70 (d, 1H). LC/MS (APCI) m/z 1040.3 (M+H)⁺.

### Example 29

### (7R,20S)-18-chloro-1-(4-fluorophenyl)-10-{[2-(2-methoxyphenyl)pyrimidin-4-yl]methoxy}-19-methyl-15-{[3-(morpholin-4-yl)oxetan-3-yl]methyl}-7,8,15,16-tetrahydro-14H-17,20-etheno-13,9-(metheno)-6-oxa-2-thia-3,5,15-triazacyclooctadeca[1,2,3-cd]indene-7-carboxylic acid

The title compound was prepared according to the procedure described in Example 23, substituting (3-morpholinooxetan-3-yl)methanamine for 2-(4,4-difluoropiperidin-1-yl)ethanamine. ¹H NMR (400 MHz, dimethyl sulfoxide-d₆) δ ppm 8.68-8.58 (m, 2H), 7.58-7.35 (m, 3H), 7.35-7.16 (m, 5H), 7.13 (td, 3H), 7.00 (dtd, 2H), 6.79 (d, 1H), 6.32 (d, 1H), 5.98 (dd, 1H), 5.13 (dd, 2H), 4.28-3.75 (m, 5H), 3.72 (s, 3H), 3.53 (t, 4H), 3.36-3.07 (m, 5H), 2.88 (dd, 1H), 2.72 (dd, 1H), 2.40 (tt, 4H), 1.77 (s, 3H). MS (ESI) m/z 930 (M+H)⁺.

### Example 30

### (7R,20S)-18-chloro-1-(4-fluorophenyl)-10-{[2-(2-methoxyphenyl)pyrimidin-4-yl]methoxy}-19-methyl-15-[(oxan-4-yl)methyl]-7,8,15,16-tetrahydro-14H-17,20-etheno-13,9-(metheno)-6-oxa-2-thia-3,5,15-triazacyclooctadeca[1,2,3-cd]indene-7-carboxylic acid

The title compound was prepared according to the procedure described in Example 23, substituting (tetrahydro-2H-pyran-4-yl)methanamine for 2-(4,4-difluoropiperidin-1-yl)ethanamine. ¹H NMR (400 MHz, dimethyl sulfoxide-d₆) δ ppm 8.65 (d, 2H), 7.68-7.39 (m, 3H), 7.37-7.17 (m, 5H), 7.13 (td, 3H), 7.02 (td, 2H), 6.90 (s, 1H), 6.50-6.36 (m, 1H), 6.10-5.84 (m, 1H), 5.29-5.01 (m, 2H), 4.12 (s, 6H), 3.86 (dt, 2H), 3.73 (s, 3H), 3.55-3.09 (m, 5H), 1.96-1.73 (m, 2H), 1.72 (s, 3H), 1.46-1.23 (m, 2H). MS (ESI) m/z 873 (M+H)⁺.

### Example 31

### (7R,20S)-15-[2-(4-acetylpiperazin-1-yl)ethyl]-18-chloro-1-(4-fluorophenyl)-10-{[2-(2-methoxyphenyl)pyrimidin-4-yl]methoxy}-19-methyl-7,8,15,16-tetrahydro-14H-17,20-etheno-13,9-(metheno)-6-oxa-2-thia-3,5,15-triazacyclooctadeca[1,2,3-cd]indene-7-carboxylic acid

The title compound was prepared according to the procedure described in Example 23, substituting 1-(4-(2-aminoethyl)piperazin-1-yl)ethanone for 2-(4,4-difluoropiperidin-1-yl)ethanamine. ¹H NMR (501 MHz, dimethyl sulfoxide-d₆) δ ppm 8.65-8.56 (m, 2H), 7.54-7.35 (m, 3H), 7.30-7.18 (m, 5H), 7.19-7.10 (m, 3H), 7.03 (t, 2H), 6.74 (d, 1H), 6.34 (d, 1H), 5.91 (dd, 1H), 5.26-4.93 (m, 2H), 3.94-3.77 (m, 9H), 3.74 (s, 3H), 3.42 (t, 2H), 3.37-3.18 (m, 6H), 3.13 (dd, 1H), 2.04 (s, 3H), 1.75 (s, 3H). MS (ESI) m/z 929 (M+H)⁺.

### Example 32

### (7R,20S)-18-chloro-1-(4-fluorophenyl)-15-{2-[(2-methoxyethyl)(methyl)amino]ethyl}-10-{[2-(2-methoxyphenyl)pyrimidin-4-yl]methoxy}-19-methyl-7,8,15,16-tetrahydro-14H-17,20-etheno-13,9-(metheno)-6-oxa-2-thia-3,5,15-triazacyclooctadeca[1,2,3-cd]indene-7-carboxylic acid

The title compound was prepared according to the procedure described in Example 23, substituting N-(2-methoxyethyl)-N-methylethane-1,2-diamine for 2-(4,4-difluoropiperidin-1-yl)ethanamine. ¹H NMR (501 MHz, dimethyl sulfoxide-d₆) δ ppm 8.64-8.57 (m, 2H), 7.53-7.38 (m, 3H), 7.25-7.15 (m, 4H), 7.13 (ddd, 3H), 7.03 (t, 2H), 6.72 (d, 1H), 6.39 (d, 1H), 5.91 (dd, 1H), 5.24-4.93 (m, 2H), 3.73 (s, 3H), 3.73-3.55 (m, 9H), 3.41 (dt, 3H), 3.30 (s, 3H), 3.27-3.12 (m, 3H), 2.90 (s, 3H), 1.70 (s, 3H). MS (ESI) m/z 890 (M+H)⁺.

### Example 33

### (7R,20S)-18-chloro-1-(4-fluorophenyl)-N-hydroxy-10-{[2-(2-methoxyphenyl)pyrimidin-4-yl]methoxy}-19-methyl-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxamide

To a solution of Example 1W (25 mg), hydroxylamine hydrochloride (2.1 mg) and 1-benzotriazolyl hydrate (4.5 mg) in N,N-dimethylformamide (0.57 mL) was added 4-methylmorpholine (0.006 mL), and the reaction was stirred at ambient temperature for 1.5 hours. The reaction was quenched by the addition of acetic acid (0.1 mL) and water (1 mL). The solution was purified by reverse-phase HPLC (Phenomenenex® Luna® C18 250×50 mm column), eluting with 5 to 85% acetonitrile in 0.1% trifluoroacetic acid/water over 30 minutes. The fractions containing product were lyophilized to give the title product. ¹H NMR (500 MHz, dimethylsulfoxide-d₆) δ ppm 10.80 (s, 1H), 8.90 (s, 1H), 8.62 (s, 1H), 8.56 (d, 1H), 7.55-7.44 (m, 4H), 7.16 (dtd, 8H), 7.08-7.03 (m, 1H), 6.79 (d, 1H), 6.61 (d, 1H), 5.98 (dd, 1H), 5.17 (d, 1H), 4.99 (d, 1H), 4.37 (s, 2H), 4.19 (s, 2H), 3.75 (s, 3H), 3.44-3.39 (m, 8H), 3.22 (dd, 1H), 3.11-3.00 (m, 4H), 2.80 (s, 3H), 1.57 (s, 3H). MS (ESI) m/z 915.4 (M+H)⁺.

### Example 34

### (7R,20S)-18-chloro-1-(4-fluorophenyl)-15-[2-(4-hydroxypiperidin-1-yl)ethyl]-10-{[2-(2-methoxyphenyl)pyrimidin-4-yl]methoxy}-19-methyl-7,8,15,16-tetrahydro-14H-17,20-etheno-13,9-(metheno)-6-oxa-2-thia-3,5,15-triazacyclooctadeca[1,2,3-cd]indene-7-carboxylic acid

### Example 34A

### (2R)-ethyl 2-((5-((1S)-3-chloro-4-(1,3-dioxan-2-yl)-2-methylphenyl)-6-(4-fluorophenyl)thieno[2,3-d]pyrimidin-4-yl)oxy)-3-(5-(((2-(4-hydroxypiperidin-1-yl)ethyl)amino)methyl)-2-((2-(2-methoxyphenyl)pyrimidin-4-yl)methoxy)phenyl)propanoate

To a mixture of Example 1T (60 mg) in dichloromethane (3 mL) and acetic acid (0.3 mL) was added 1-(2-aminoethyl)piperidin-4-ol (10 mg). The mixture was stirred at room temperature for 30 minutes before the addition of sodium triacetoxyborohydride (44 mg). The mixture was stirred at room temperature for 2 hours. The mixture was diluted with ethyl acetate (200 mL), washed with saturated aqueous sodium bicarbonate mixture and brine, and dried over sodium sulfate. Filtration and evaporation of the solvent provided the title compound, which was used in the subsequent step without further purification. MS (ESI) m/z 1003.64 (M+H)⁺.

### Example 34B

### (7R,20S)-18-chloro-1-(4-fluorophenyl)-15-[2-(4-hydroxypiperidin-1-yl)ethyl]-10-{[2-(2-methoxyphenyl)pyrimidin-4-yl]methoxy}-19-methyl-7,8,15,16-tetrahydro-14H-17,20-etheno-13,9-(metheno)-6-oxa-2-thia-3,5,15-triazacyclooctadeca[1,2,3-cd]indene-7-carboxylic acid

To a mixture of Example 34A (73 mg) in dichloromethane (6 mL) and trifluoroacetic acid (1 mL) was added a few drops of water. The mixture was stirred at room temperature for 4 hours. The mixture was concentrated under vacuum, and the residue was diluted with ethyl acetate (200 mL) and washed with saturated aqueous sodium bicarbonate mixture and brine and dried over sodium sulfate. Filtration and evaporation of the solvent gave a residue that was dissolved in dichloromethane (4 mL). Magnesium sulfate (anhydrous, 1 g) was added. The mixture was stirred at room temperature for 1 hour before the addition of sodium triacetoxyborohydride (232 mg). The mixture was stirred further for 1 hour. The reaction mixture was partitioned between ethyl acetate (300 mL) and saturated aqueous sodium bicarbonate mixture (100 mL). The organic layer was washed with brine and dried over sodium sulfate. Filtration and evaporation of the solvent gave a residue that was dissolved in tetrahydrofuran/methanol/water (2:1:1, 4 mL). Lithium hydroxide monohydrate (50 mg) was added. The mixture was stirred at room temperature for 3 hours. The solvent was evaporated under vacuum, and the residue was dissolved in N,N-dimethylformamide (10 mL) and neutralized with trifluoroacetic acid (0.5 mL). The mixture was purified by reverse phase chromatography on a Gilson HPLC (Phenomenex®, 250×50 mm, C18 column), eluting with 20% acetonitrile in 0.1% trifluoroacetic acid in water to 75% acetonitrile in 0.1% trifluoroacetic acid in water over 35 minutes to provide the title compound. ¹H NMR (500 MHz, dimethyl sulfoxide-d₆) δ ppm 8.65-8.54 (m, 2H), 7.50 (d, 1H), 7.45 (t, 1H), 7.33-7.26 (m, 1H), 7.23 (dd, 2H), 7.19-7.10 (m, 3H), 7.03 (t, 1H), 6.88 (d, 1H), 6.81 (d, 1H), 6.75 (d, 1H), 6.54 (d, 1H), 6.43 (d, 1H), 5.87 (dd, 1H), 5.22-5.09 (m, 2H), 4.18 (d, 1H), 3.76 (d, 6H), 3.24-3.09 (m, 2H), 2.45 (s, 3H). MS (ESI) m/z 901.3 (M+H)⁺.

### Example 35

### (7R,21S)-19-chloro-1-(4-fluorophenyl)-20-methyl-15-oxo-16-{2-[4-(2,2,2-trifluoroethyl)piperazin-1-yl]ethyl}-10-{[2-(3,3,3-trifluoropropoxy)pyrimidin-4-yl]methoxy}-7,8,14,15,16,17-hexahydro-18,21-etheno-13,9-(metheno)-6-oxa-2-thia-3,5,16-triazacyclononadeca[1,2,3-cd]indene-7-carboxylic acid

### Example 35A

### tert-butyl (2-(4-(2,2,2-trifluoroethyl)piperazin-1-yl)ethyl)carbamate

To a mixture of tert-butyl (2-(piperazin-1-yl)ethyl)carbamate (500 mg) in tetrahydrofuran (16 mL) was added triethylamine (221 mg) followed by 2,2,2-trifluoroethyl trifluoromethanesulfonate (506 mg). The reaction mixture was stirred at 60° C. overnight, and concentrated under reduced pressure. The residue was dissolved in ethyl acetate, washed with water and brine, dried over anhydrous sodium sulfate, filtered, and concentrated. The residue was purified by silica gel chromatography on an AnaLogix IntelliFlash²⁸⁰ system (5-18% methanol in dichloromethane, linear gradient) to provide the title compound. MS (ESI) m/z 312.1 (M+H)⁺.

### Example 35B

### 2-(4-(2,2,2-trifluoroethyl)piperazin-1-yl)ethanamine

To a mixture of Example 35A (100 mg) in dichloromethane (0.5 mL) was added trifluoroacetic acid (0.5 mL). The reaction mixture was stirred at ambient temperature for 20 minutes and was concentrated under reduced pressure. The crude product was used in the next step without further purification. LC/MS (APCI) m/z 212.4 (M+H)⁺.

### Example 35C

### 2-chloro-3-methyl-4-(4,4,5,5-tetramethyl-1,3,2-dioxaborolan-2-yl)benzaldehyde

Oven dried potassium acetate (4.20 g), bis(pinacolato)diboron (5.98 g), Example 10A (5 g, 21.41 mmol) and 1,1′-bis(diphenylphosphino)ferrocenedichloro palladium(II) dichloromethane complex (0.392 g) were all placed into an oven-dried 500 mL round-bottom flask. A dried vigeroux column was added, and the system was inerted with argon for 45 minutes. In the meantime, 2-methyltetrahydrofuran (107 mL) was sparged with argon for 40 minutes and was transferred to the reaction flask containing the material. The mixture was stirred at 90° C. (external), which refluxed the reaction. After 5 hours, the reaction mixture was cooled to room temperature and was filtered through diatomaceous earth. The filtrate was stirred with charcoal and thiosilica gel for 30 minutes and was filtered through a small pad of silica gel to provide a much lighter filtrate, which was concentrated by rotary evaporation. The material was taken up in dichloromethane and purified by silica gel chromatography (Grace system, 120 g RediSep® Gold, 0-50% ethyl acetate:heptanes over 30 minutes) to provide the title compound. ¹H NMR (400 MHz, chloroform-d) δ ppm 10.56 (t, 1H), 7.80-7.65 (m, 2H), 2.65 (d, 3H), 1.38 (d, 13H).

### Example 35D

### (2R)-ethyl 3-(5-(2-(tert-butoxy)-2-oxoethyl)-2-((2-(3,3,3-trifluoropropoxy)pyrimidin-4-yl)methoxy)phenyl)-2-((5-((1S)-3-chloro-4-formyl-2-methylphenyl)-6-(4-fluorophenyl)thieno[2,3-d]pyrimidin-4-yl)oxy)propanoate

Example 7M (1000 mg), Example 35C (403 mg), 4-(di-tert-butylphosphino)-N,N-dimethylaniline (19.05 mg), tris(dibenzylideneacetone)dipalladium(0) (32.9 mg) and cesium carbonate (585 mg) were placed in a 25 mL pressure vial. The material was sparged for 60 minutes by blowing nitrogen over the material while stirring. Meanwhile, anhydrous 1,4-dioxane and water were respectively sparged with stirring for 60 minutes by bubbling nitrogen through them. The sparged 1,4-dioxane (8.0 mL) and water (1.0 mL) were respectively transferred via cannula to the vial with the material. The reaction mixture was stirred at 40° C. for 1 day. The reaction mixture was filtered through diatomaceous earth and was washed with dichloromethane. The filtrate was concentrated and was purified by silica gel chromatography on an AnaLogix IntelliFlash²⁸⁰ system eluting with 5-65% ethyl acetate in hexanes to provide the title compound. LC/MS (APCI) m/z 909.2 (M+H)⁺.

### Example 35E

### (2R)-ethyl 3-(5-(2-(tert-butoxy)-2-oxoethyl)-2-((2-(3,3,3-trifluoropropoxy)pyrimidin-4-yl)methoxy)phenyl)-2-((5-((1S)-3-chloro-2-methyl-4-(((2-(4-(2,2,2-trifluoroethyl)piperazin-1-yl)ethyl)amino)methyl)phenyl)-6-(4-fluorophenyl)thieno[2,3-d]pyrimidin-4-yl)oxy)propanoate

A pH 4 buffer mixture was prepared by dissolving 48 g of acetic acid and 36 g of sodium acetate tris hydrate in methanol and adding methanol to reach a volume of 1 L. A mixture of Example 35D (100 mg) and Example 35B (54.8 mg) in 1.0 mL of acetic acid/sodium acetate pH 4 methanol mixture was stirred at ambient temperature for 25 minutes. Sodium cyanoborohydride (8.29 mg) was added. The mixture was stirred at ambient temperature for 45 minutes. The mixture was concentrated and was purified by silica gel chromatography on an AnaLogix IntelliFlash²⁸⁰ system (1-5% methanol in dichloromethane, linear gradient) to provide the title compound. MS (ESI) m/z 1104.3 (M+H)⁺.

### Example 35F

### 2-(3-((2R)-2-((5-((1S)-3-chloro-2-methyl-4-(((2-(4-(2,2,2-trifluoroethyl)piperazin-1-yl)ethyl)amino)methyl)phenyl)-6-(4-fluorophenyl)thieno[2,3-d]pyrimidin-4-yl)oxy)-3-ethoxy-3-oxopropyl)-4-((2-(3,3,3-trifluoropropoxy)pyrimidin-4-yl)methoxy)phenyl)acetic acid

To a mixture of Example 35E (45 mg) in dichloromethane (0.5 mL) was added trifluoroacetic acid (0.5 mL). The reaction mixture was stirred at ambient temperature for 50 minutes, and was concentrated under reduced pressure. The crude product was used in the next step without further purification. LC/MS (APCI) m/z 1048.3 (M+H)⁺.

### Example 35G

### ethyl (7R,21S)-19-chloro-1-(4-fluorophenyl)-20-methyl-15-oxo-16-{2-[4-(2,2,2-trifluoroethyl)piperazin-1-yl]ethyl}-10-{[2-(3,3,3-trifluoropropoxy)pyrimidin-4-yl]methoxy}-7,8,14,15,16,17-hexahydro-18,21-etheno-13,9-(metheno)-6-oxa-2-thia-3,5,16-triazacyclononadeca[1,2,3-cd]indene-7-carboxylate

Example 35F (51 mg) was dissolved in dichloromethane (4 mL). Then 1-bis(dimethylamino)methylene]-1H-1,2,3-triazolo[4,5-b]pyridinium-3-oxid hexafluorophosphate (18.83 mg), 1-hydroxybenzotriazole hydrate (3.79 mg), 4-dimethylaminopyridine (4.03 mg) and N,N-diisopropylethylamine (0.034 mL) were added. The reaction mixture was stirred at ambient temperature for 1 hour. The mixture was diluted with ethyl acetate and washed with water. The organics were dried over anhydrous sodium sulfate, filtered, and concentrated. The residue was purified by silica gel chromatography on an AnaLogix IntelliFlash²⁸⁰ system (1-5% methanol in dichloromethane, linear gradient) to provide the title compound. LC/MS (APCI) m/z 1031.1 (M+H)⁺.

### Example 35H

### (7R,21S)-19-chloro-1-(4-fluorophenyl)-20-methyl-15-oxo-16-{2-[4-(2,2,2-trifluoroethyl)piperazin-1-yl]ethyl}-10-{[2-(3,3,3-trifluoropropoxy)pyrimidin-4-yl]methoxy}-7,8,14,15,16,17-hexahydro-18,21-etheno-13,9-(metheno)-6-oxa-2-thia-3,5,16-triazacyclononadeca[1,2,3-cd]indene-7-carboxylic acid

To a mixture of Example 35G (18 mg) in tetrahydrofuran (0.26 mL) and methanol (0.26 mL) was added lithium hydroxide (0.262 mL, 1.0 M in water). The mixture was stirred at ambient temperature for 5 hours and was concentrated under reduced pressure. The residue was dissolved in N,N-dimethylformamide (1 mL) and was acidified with trifluoroacetic acid. The mixture was purified on a Gilson prep HPLC (Zorbax, C-18, 250×21.2 mm column, 5 to 90% acetonitrile in water (0.1% trifluoroacetic acid)) to provide the title compound. ¹H NMR (501 MHz, dimethyl sulfoxide-d₆) δ ppm 9.31 (s, 1H), 8.52-8.41 (m, 2H), 7.26 (t, 2H), 7.15 (t, 2H), 7.04 (dd, 1H), 6.92-6.75 (m, 2H), 6.72 (d, 1H), 6.64 (s, 1H), 4.89 (d, 1H), 4.65 (d, 1H), 4.48 (dq, 5H), 3.87 (d, 1H), 3.77-3.24 (m, 9H), 3.22-3.02 (m, 5H), 2.88-2.64 (m, 5H), 1.84 (s, 3H). MS (ESI) m/z 1002.3 (M+H)⁺.

### Example 36

### (7R,21R)-19-chloro-1-(4-fluorophenyl)-20-methyl-15-oxo-16-{2-[4-(2,2,2-trifluoroethyl)piperazin-1-yl]ethyl}-10-{[2-(3,3,3-trifluoropropoxy)pyrimidin-4-yl]methoxy}-7,8,14,15,16,17-hexahydro-18,21-etheno-13,9-(metheno)-6-oxa-2-thia-3,5,16-triazacyclononadeca[1,2,3-cd]indene-7-carboxylic acid

The title compound was isolated during the synthesis of Example 35G. ¹H NMR (501 MHz, dimethyl sulfoxide-d₆) δ ppm 9.19 (s, 1H), 8.49 (s, 1H), 8.28 (s, 1H), 7.29-7.23 (m, 2H), 7.21-7.12 (m, 2H), 7.02 (dd, 1H), 6.75 (d, 2H), 6.50 (d, 2H), 6.04 (d, 1H), 5.13 (s, 1H), 4.99 (d, 1H), 4.78 (s, 1H), 4.56 (d, 1H), 4.48 (td, 2H), 4.36 (s, 1H), 3.96 (s, 1H), 3.70-3.21 (m, 8H), 3.09 (d, 5H), 2.87-2.63 (m, 6H), 2.31 (s, 3H). MS (ESI) m/z 1002.2 (M+H)⁺.

### Example 37

### (7R,20S)-18-chloro-15-[2-(dimethylamino)ethyl]-1-(4-fluorophenyl)-10-{[2-(2-methoxyphenyl)pyrimidin-4-yl]methoxy}-19-methyl-7,8,15,16-tetrahydro-14H-17,20-etheno-13,9-(metheno)-6-oxa-2-thia-3,5,15-triazacyclooctadeca[1,2,3-cd]indene-7-carboxylic acid

The title compound was prepared according to the procedure described in Example 23, substituting N¹, N¹-dimethylethane-1,2-diamine for 2-(4,4-difluoropiperidin-1-yl)ethanamine. ¹H NMR (400 MHz, dimethyl sulfoxide-d₆) δ ppm 8.62-8.55 (m, 2H), 7.52-7.39 (m, 4H), 7.25-7.17 (m, 3H), 7.17-7.07 (m, 5H), 7.04-6.93 (m, 2H), 6.70 (d, 1H), 6.40 (d, 1H), 5.91 (dd, 1H), 5.19-4.88 (m, 2H), 3.77 (q, 3H), 3.72 (s, 3H), 3.63-3.47 (m, 1H), 3.45-3.25 (m, 2H), 3.26-3.01 (m, 3H), 2.87 (s, 6H), 1.68 (s, 3H). MS (ESI) m/z 846 (M+H)⁺.

### Example 38

### (7R,20S)-18-chloro-1-(4-fluorophenyl)-15-(3-hydroxypropyl)-10-{[2-(2-methoxyphenyl)pyrimidin-4-yl]methoxy}-19-methyl-7,8,15,16-tetrahydro-14H-17,20-etheno-13,9-(metheno)-6-oxa-2-thia-3,5,15-triazacyclooctadeca[1,2,3-cd]indene-7-carboxylic acid

### Example 38A

### ethyl (R)-2-((5-((1S)-3-chloro-4-(1,3-dioxan-2-yl)-2-methylphenyl)-6-(4-fluorophenyl)thieno[2,3-d]pyrimidin-4-yl)oxy)-3-(5-(((3-hydroxypropyl)amino)methyl)-2-((2-(2-methoxyphenyl)pyrimidin-4-yl)methoxy)phenyl)propanoate

To a mixture of Example 1T (520 mg) in dichloromethane (10 mL) and acetic acid (0.5 mL) was added 3-amino-1-propanol (134 mg). The mixture was stirred at room temperature for 30 minutes before the addition of sodium triacetoxyborohydride (378 mg). The mixture was stirred at room temperature for 2 hours. LC/MS showed the expected product as a major peak. The mixture was diluted with ethyl acetate (200 mL), washed with saturated aqueous sodium bicarbonate mixture and brine, and dried over sodium sulfate. Filtration and evaporation of the solvent provided the title compound, which was used in the next step without further purification. MS (ESI) m/z 934.2 (M+H)⁺.

### Example 38B

### (7R,20S)-18-chloro-1-(4-fluorophenyl)-15-(3-hydroxypropyl)-10-{[2-(2-methoxyphenyl)pyrimidin-4-yl]methoxy}-19-methyl-7,8,15,16-tetrahydro-14H-17,20-etheno-13,9-(metheno)-6-oxa-2-thia-3,5,15-triazacyclooctadeca[1,2,3-cd]indene-7-carboxylic acid

The title compound was prepared as described in Example 34B, replacing Example 34A with Example 38A. ¹H NMR (500 MHz, dimethyl sulfoxide-d₆) δ ppm 8.73-8.57 (m, 2H), 7.58 (s, 2H), 7.54-7.44 (m, 4H), 7.21-7.13 (m, 6H), 7.09-7.02 (m, 4H), 6.91 (d, 1H), 6.55 (d, 1H), 6.01 (s, 1H), 5.31-5.02 (m, 2H), 4.22 (d, 20H), 3.76 (s, 3H), 3.64 (s, 4H), 3.20 (d, 2H), 2.89 (s, 3H), 2.73 (s, 3H). MS (ESI) m/z 832.2 (M+H)⁺.

### Example 39

### (7R,20S)-18-chloro-1-(4-fluorophenyl)-10-{[2-(2-methoxyphenyl)pyrimidin-4-yl]methoxy}-15,19-dimethyl-7,8,15,16-tetrahydro-14H-17,20-etheno-13,9-(metheno)-6-oxa-2-thia-3,5,15-triazacyclooctadeca[1,2,3-cd]indene-7-carboxylic acid

### Example 39A

### (2R)-ethyl 2-((5-((1S)-3-chloro-4-formyl-2-methylphenyl)-6-(4-fluorophenyl)thieno[2,3-d]pyrimidin-4-yl)oxy)-3-(5-(((3-hydroxypropyl)amino)methyl)-2-((2-(2-methoxyphenyl)pyrimidin-4-yl)methoxy)phenyl)propanoate

Example 38A (320 mg) was dissolved in a mixture of trifluoroacetic acid/tetrahydrofuran/water (3/3/0.5). The reaction mixture was stirred at room temperature for 3 hours. The mixture was concentrated under vacuum, and the residue was dissolved in ethyl acetate (200 mL), washed with saturated aqueous sodium bicarbonate mixture and brine, and dried over sodium sulfate. Filtration and evaporation of the solvent provided the title compound, which was used in the next step without further purification. MS (ESI) m/z 934.2 (M+H)⁺.

### Example 39B

### ethyl (7R,20S)-18-chloro-1-(4-fluorophenyl)-15-(3-hydroxypropyl)-10-{[2-(2-methoxyphenyl)pyrimidin-4-yl]methoxy}-19-methyl-7,8,15,16-tetrahydro-14H-17,20-etheno-13,9-(metheno)-6-oxa-2-thia-3,5,15-triazacyclooctadeca[1,2,3-cd]indene-7-carboxylate

Example 39A (320 mg) was dissolved in dichloromethane (10 mL) and anhydrous magnesium sulfate (1.75 g) was added. The mixture was stirred at room temperature for 1 hour before the addition of sodium triacetoxyborohydride (232 mg). The mixture was stirred further for 1 hour. The reaction mixture was added to a ethyl acetate (300 mL) and saturated aqueous sodium bicarbonate mixture (100 mL). The organic layer was washed with brine and dried over sodium sulfate. Filtration and evaporation of solvent provided the title compound. MS (ESI) m/z 860.1 (M+H)⁺.

### Example 39C

### ethyl (7R,20S)-18-chloro-1-(4-fluorophenyl)-10-{[2-(2-methoxyphenyl)pyrimidin-4-yl]methoxy}-15,19-dimethyl-7,8,15,16-tetrahydro-14H-17,20-etheno-13,9-(metheno)-6-oxa-2-thia-3,5,15-triazacyclooctadeca[1,2,3-cd]indene-7-carboxylate

To a mixture of dimethyl sulfoxide (0.5 mL) in dichloromethane (5 mL) at −78° C. was added oxalyl chloride (0.2 mL). The mixture was stirred 20 minutes at −78° C., and a mixture of Example 39B (300 mg) in dichloromethane (5 mL) was added through a syringe. After 40 minutes, triethylamine (0.5 mL) was added to the mixture. The mixture was stirred overnight, and the temperature was allowed to rise to room temperature. The reaction mixture was diluted with ethyl acetate (200 mL), washed with water and brine, and dried over sodium sulfate. Filtration and evaporation of the solvent provided the title compound as a minor component, which was used without further purification. MS (ESI) m/z 858.1 (M+H)⁺.

### Example 39D

### (7R,20S)-18-chloro-1-(4-fluorophenyl)-10-{[2-(2-methoxyphenyl)pyrimidin-4-yl]methoxy}-15,19-dimethyl-7,8,15,16-tetrahydro-14H-17,20-etheno-13,9-(metheno)-6-oxa-2-thia-3,5,15-triazacyclooctadeca[1,2,3-cd]indene-7-carboxylic acid

To a mixture of Example 39C (256 mg) in tetrahydrofuran (10 mL) and methanol (5 mL) and water (5 mL) was added LiOH monohydrate (120 mg). The mixture was stirred for 20 minutes at 0° C. The reaction mixture was acidified with trifluoroacetic acid and was concentrated under vacuum. The residue was dissolved in N,N-dimethylformamide (12 mL) and was purified by reverse-phase chromatography on a Gilson HPLC (Phenomenex®, 250×50 mm, C18 column), eluting with 20 to 75% acetonitrile in water (0.1% trifluoroacetic acid) to provide the title compound. ¹H NMR (400 MHz, dimethyl sulfoxide-d₆) δ ppm 8.69-8.58 (m, 2H), 7.60-7.43 (m, 5H), 7.37-7.10 (m, 11H), 7.05 (t, 1H), 6.88 (d, 1H), 6.66 (s, 1H), 6.09-5.98 (m, 1H), 5.30-4.99 (m, 3H), 4.68-4.18 (m, 4H), 3.76 (s, 3H), 3.21 (s, 3H), 1.64 (s, 3H). MS (ESI) m/z 788.2 (M+H)⁺.

### Example 40

### (7R,20S)-18-chloro-1-(4-fluorophenyl)-10-{[2-(2-methoxyphenyl)pyrimidin-4-yl]methoxy}-19-methyl-7,8,15,16-tetrahydro-14H-17,20-etheno-13,9-(metheno)-6-oxa-2-thia-3,5,15-triazacyclooctadeca[1,2,3-cd]indene-7-carboxylic acid

### Example 40A

### ethyl (7R,20S)-18-chloro-1-(4-fluorophenyl)-10-{[2-(2-methoxyphenyl)pyrimidin-4-yl]methoxy}-19-methyl-7,8,15,16-tetrahydro-14H-17,20-etheno-13,9-(metheno)-6-oxa-2-thia-3,5,15-triazacyclooctadeca[1,2,3-cd]indene-7-carboxylate

Example 40A was isolated as a minor product during the synthesis of Example 39C. MS (ESI) m/z 802.2 (M+H)⁺.

### Example 40B

### (7R,20S)-18-chloro-1-(4-fluorophenyl)-10-{[2-(2-methoxyphenyl)pyrimidin-4-yl]methoxy}-19-methyl-7,8,15,16-tetrahydro-14H-17,20-etheno-13,9-(metheno)-6-oxa-2-thia-3,5,15-triazacyclooctadeca[1,2,3-cd]indene-7-carboxylic acid

To a mixture of Example 40A (256 mg) in tetrahydrofuran (10 mL), methanol (5 mL) and water (5 mL) was added LiOH (120 mg). The mixture was stirred for 20 minutes at 0° C. The reaction mixture was acidified with trifluoroacetic acid and was concentrated under vacuum. The residue was dissolved in N,N-dimethylformamide (12 mL) and was purified by reverse-phase chromatography on Gilson HPLC (Phenomenex®, 250×50 mm, C18 column), eluting with 20 to 75% acetonitrile in water (0.1% trifluoroacetic acid) over 35 minutes to provide the title compound. ¹H NMR (501 MHz, dimethyl sulfoxide-d₆) δ ppm 9.67 (s, 2H), 8.75 (d, 1H), 8.71 (s, 1H), 7.54 (dd, 1H), 7.52-7.46 (m, 2H), 7.37 (dd, 1H), 7.32-7.25 (m, 4H), 7.23-7.13 (m, 3H), 7.09-6.97 (m, 2H), 6.27 (d, 1H), 6.12 (dd, 1H), 5.37-5.09 (m, 2H), 4.36 (dd, 2H), 4.09 (d, 1H), 3.77 (s, 5H), 3.18 (dd, 1H), 1.94 (s, 3H). MS (ESI) m/z 774.1 (M+H)⁺.

### Example 41

### (7R,20S)-18-chloro-15-[2-(4-cyclopropylpiperazin-1-yl)ethyl]-1-(4-fluorophenyl)-10-{[2-(2-methoxyphenyl)pyrimidin-4-yl]methoxy}-19-methyl-7,8,15,16-tetrahydro-14H-17,20-etheno-13,9-(metheno)-6-oxa-2-thia-3,5,15-triazacyclooctadeca[1,2,3-cd]indene-7-carboxylic acid

### Example 41A

### (2R)-ethyl 2-((5-((1S)-3-chloro-4-(1,3-dioxan-2-yl)-2-methylphenyl)-6-(4-fluorophenyl)thieno[2,3-d]pyrimidin-4-yl)oxy)-3-(5-(((2-hydroxyethyl)amino)methyl)-2-((2-(2-methoxyphenyl)pyrimidin-4-yl)methoxy)phenyl)propanoate

To a mixture of Example 1T (300 mg) in dichloromethane (6 mL) and acetic acid (0.5 mL) was added ethanolamine (64 mg). The mixture was stirred at room temperature for 30 minutes before the addition of sodium triacetoxyborohydride (220 mg). The mixture was stirred at room temperature for 2 hours. The mixture was diluted with ethyl acetate (200 mL), washed with saturated aqueous sodium bicarbonate mixture and brine, and dried over sodium sulfate. Filtration and evaporation of the solvent provided the title compound, which was used in the last step without further purification. MS (ESI) m/z 920.1 (M+H)⁺.

### Example 41B

### (2R)-ethyl 3-(5-(((tert-butoxycarbonyl)(2-hydroxyethyl)amino)methyl)-2-((2-(2-methoxyphenyl)pyrimidin-4-yl)methoxy)phenyl)-2-((5-((1S)-3-chloro-4-(1,3-dioxan-2-yl)-2-methylphenyl)-6-(4-fluorophenyl)thieno[2,3-d]pyrimidin-4-yl)oxy)propanoate

To a mixture of Example 41A (400 mg) in dichloromethane (10 mL) was added di-tert-butyldicarbonate (190 mg). The mixture was stirred at room temperature overnight. The mixture was diluted with ethyl acetate (200 mL) and washed with aqueous 1N HCl mixture, saturated aqueous sodium bicarbonate mixture, and brine, and dried over sodium sulfate. Filtration and evaporation of the solvent provided the title compound, which was used in the next step without further purification. MS (ESI) m/z 1020.33 (M+H)⁺.

### Example 41C

### (2R)-ethyl 3-(5-(((tert-butoxycarbonyl)(2-oxoethyl)amino)methyl)-2-((2-(2-methoxyphenyl)pyrimidin-4-yl)methoxy)phenyl)-2-((5-((1S)-3-chloro-4-(1,3-dioxan-2-yl)-2-methylphenyl)-6-(4-fluorophenyl)thieno[2,3-d]pyrimidin-4-yl)oxy)propanoate

To a mixture of dimethylsulfoxide (0.5 mL) in dichloromethane (5 mL) at −78° C. was added oxalyl chloride (0.2 mL). The mixture was stirred for 20 minutes at −78° C., and a mixture of Example 41B (650 mg) in dichloromethane (10 mL) was added through a syringe. After 40 minutes, triethylamine (0.5 mL) was added to the mixture, and the mixture was stirred overnight, as the temperature was allowed to rise to room temperature. The reaction mixture was diluted with ethyl acetate (200 mL) and washed with water and brine, and dried over sodium sulfate. Filtration and evaporation of the solvent provided the title compound, which was used in the next step without further purification. MS (ESI) m/z 1018.0 (M+H)⁺.

### Example 41D

### (2R)-ethyl 2-((5-((1S)-3-chloro-4-formyl-2-methylphenyl)-6-(4-fluorophenyl)thieno[2,3-d]pyrimidin-4-yl)oxy)-3-(5-(((2-(4-cyclopropylpiperazin-1-yl)ethyl)amino)methyl)-2-((2-(2-methoxyphenyl)pyrimidin-4-yl)methoxy)phenyl)propanoate

To a mixture of Example 41C (53 mg) in dichloromethane (2 mL) was added 1-cyclopropylpiperazine (24 mg). The mixture was stirred for 20 minutes at room temperature before the addition of sodium triacetoxyborohydride (33 mg). The mixture was stirred at room temperature for 40 minutes. The reaction mixture was diluted with ethyl acetate (200 mL), washed with water and brine, and dried over sodium sulfate. Filtration and evaporation of the solvent provided the title compound, which was used in the next reaction without further purification. MS (ESI) m/z 1027.4 (M+H)⁺.

### Example 41E

### (7R,20S)-18-chloro-15-[2-(4-cyclopropylpiperazin-1-yl)ethyl]-1-(4-fluorophenyl)-10-{[2-(2-methoxyphenyl)pyrimidin-4-yl]methoxy}-19-methyl-7,8,15,16-tetrahydro-14H-17,20-etheno-13,9-(metheno)-6-oxa-2-thia-3,5,15-triazacyclooctadeca[1,2,3-cd]indene-7-carboxylic acid

The title compound was prepared as described in Example 34B, replacing Example 34A with Example 41D. ¹H NMR (400 MHz, dimethyl sulfoxide-d₆) δ ppm 8.65 (d, 1H), 7.58-7.44 (m, 3H), 7.34-7.11 (m, 7H), 7.05 (t, 1H), 6.86-6.77 (m, 4H), 6.46-6.39 (m, 3H), 5.94 (dd, 1H), 5.24-5.00 (m, 2H), 4.14 (s, 2H), 3.46-2.94 (m, 18H), 1.76 (s, 3H), 1.24 (s, 1H), 0.69-0.53 (m, 5H). MS (ESI) m/z 926.3 (M+H)⁺.

### Example 42

### (7R,20S)-18-chloro-10-{[2-(2-methoxyphenyl)pyrimidin-4-yl]methoxy}-19-methyl-15-[2-(4-methylpiperazin-1-yl)ethyl]-1-(prop-1-yn-1-yl)-7,8,15,16-tetrahydro-14H-17,20-etheno-13,9-(metheno)-6-oxa-2-thia-3,5,15-triazacyclooctadeca[1,2,3-cd]indene-7-carboxylic acid

### Example 42A

### 5,6-diiodothieno[2,3-d]pyrimidin-4(3H)-one

A 4-neck 2 L round-bottom flask was fitted with mechanical stirring, reflux condenser and thermocouple/JKEM and placed in an ice bath. Acetic acid (175 mL), sulfuric acid (5.18 mL) and water (36 mL) were added with stirring. The internal temperature was about 14° C. Example 1A (50 g), periodic acid (20.9 g) and iodine (48 g) were added sequentially, and the mixture was slightly endothermic. The ice bath was removed. A heating mantle was added, and the reaction mixture was heated to 60° C. and was stirred for 1 hour. Midway through, the temperature climbed to 68-69° C. The heating mantle was removed and the temperature remained at 68-70° C. without external heating (caution). LC/MS of an aliquot indicated a single peak corresponding to product. The reaction mixture was cooled to room temperature (placed in ice bath again to expedite), and the resulting suspension was filtered, washed with 5:1 acetic acid:water (three times) and diethyl ether (five times) to provide the title compound. ¹H NMR (400 MHz, dimethyl sulfoxide-d₆) δ ppm 12.60 (s, 1H), 8.13 (d, 1H). MS (ESI) m/z 405.0 (M+H)⁺.

### Example 42B

### 4-chloro-5,6-diiodothieno[2,3-d]pyrimidine

A 250 mL flask fitted with magnetic stirring, heating mantle, temperature probe and reflux condenser to a nitrogen bubbler was charged with phosphorus oxychloride (57.3 mL) and N,N-dimethylaniline (17.64 mL). To the mixture was added Example 42A (56.22 g) over 5 minutes. The resulting suspension was heated to 105° C., whereupon the reaction became difficult to stir. The mixture was heated for 0.5 hour, and the heat was turned off. The material was broken up as well as possible and transferred to a Buchner funnel with heptanes. The material was pressed down and washed with heptanes until most of the very dark color was filtered into a filter flask, leaving a lighter material. The material was scooped slowly into rapidly stirring ice cooled water (1.2° C., 600 mL) and the mixture was stirred for 15 minutes. The suspension was filtered, and the material was washed with water and separately with diethyl ether (200 mL). The material was air-dried to provide the title compound, which was used the next step without further purification. ¹H NMR (500 MHz, dimethyl sulfoxide-d₆) δ ppm 8.9 (s, 1H).

### Example 42C

### 4-chloro-5-iodo-6-(prop-1-yn-1-yl)thieno[2,3-d]pyrimidine

Example 42B (22 g), copper(i) iodide (0.992 g) and bis(triphenylphosphine)palladium dichloride (1.828 g) were inerted with argon gas in a round-bottom flask for about 20 minutes. N,N-diisopropylamine (207 mL) was added, and the mixture was sparged with argon for about 10 minutes. Prop-1-yne (2.087 g) was bubbled through the reaction, and the reaction mixture was stirred overnight under argon. The reaction mixture was concentrated, and the material was triturated with water, filtered and air-dried to provide the title compound. MS (DCI) m/z 334.8 (M+H)⁺.

### Example 42D

### (R)-ethyl 3-(5-formyl-2-((2-(2-methoxyphenyl)pyrimidin-4-yl)methoxy)phenyl)-2-((5-iodo-6-(prop-1-yn-1-yl)thieno[2,3-d]pyrimidin-4-yl)oxy)propanoate

A mixture of Example 10 (865 mg), cesium carbonate (323 mg) and Example 42C (663 mg) in 20 mL tert-butanol was heated to 65° C. for 3 hours. The reaction mixture was cooled to room temperature and partitioned between water and ethyl acetate. The aqueous phase was extracted with ethyl acetate. The combined organic phases were washed with brine, dried over magnesium sulfate, and filtered. The filtrate was concentrated, and the residue was purified by silica gel chromatography, eluting with 40-80% ethyl acetate in heptanes, to provide the title compound. MS (ESI) m/z 735.0 (M+H)⁺.

### Example 42E

### (2R)-ethyl 2-((5-((1S)-3-chloro-4-(1,3-dioxan-2-yl)-2-methylphenyl)-6-(prop-1-yn-1-yl)thieno[2,3-d]pyrimidin-4-yl)oxy)-3-(5-formyl-2-((2-(2-methoxyphenyl)pyrimidin-4-yl)methoxy)phenyl)propanoate

A round-bottom flask charged with Example 42D (760 mg), Example 1S (420 mg), cesium carbonate (1011 mg) and bis(di-tert-butyl(4-dimethylaminophenyl)phosphine)dichloropalladium(II) (73.3 mg) was evacuated and backfilled with nitrogen for 2 cycles. Anhydrous tetrahydrofuran (12 mL) and degassed water (4 mL) were added. The resulting mixture was sparged with nitrogen for 10 minutes and was heated at 65° C. for 5 hours. The mixture was partitioned between ethyl acetate and brine. The aqueous phase was extracted with ethyl acetate. The combined organic phases were dried over magnesium sulfate and filtered. The filtrate was concentrated, and the residue was purified by silica gel chromatography, eluting with 60-90% ethyl acetate in heptanes, to provide the title compound. MS (ESI) m/z 819.2 (M+H)⁺.

### Example 42F

### (2R)-ethyl 2-((5-((1S)-3-chloro-4-formyl-2-methylphenyl)-6-(prop-1-yn-1-yl)thieno[2,3-d]pyrimidin-4-yl)oxy)-3-(5-formyl-2-((2-(2-methoxyphenyl)pyrimidin-4-yl)methoxy)phenyl)propanoate

A mixture of Example 42E (670 mg) in 6 mL dichloromethane was treated with 10 mL trifluoroacetic acid and 20 drops of water at room temperature. The resulting mixture was stirred at room temperature for 3 hours. The reaction mixture was concentrated. The mixture was cooled with an ice-water bath, and the residue was slowly neutralized with saturated aqueous sodium bicarbonate mixture. The mixture was partitioned between brine and ethyl acetate. The aqueous phase was extracted with ethyl acetate. The combined organic phases were dried over magnesium sulfate and filtered. The filtrate was concentrated to provide the title compound, which was used without further purification. MS (ESI) m/z 761.2 (M+H)⁺.

### Example 42G

### ethyl (7R,20S)-18-chloro-10-{[2-(2-methoxyphenyl)pyrimidin-4-yl]methoxy}-19-methyl-15-[2-(4-methylpiperazin-1-yl)ethyl]-1-(prop-1-yn-1-yl)-7,8,15,16-tetrahydro-14H-17,20-etheno-13,9-(metheno)-6-oxa-2-thia-3,5,15-triazacyclooctadeca[1,2,3-cd]indene-7-carboxylate

To a mixture of Example 42F (100 mg) in 13 mL dichloromethane at 0° C. were added 50 mg 4 Å molecular sieves and sodium triacetoxyborohydride (84 mg) followed by 2-(4-methylpiperazin-1-yl)ethanamine (19.68 μL). The mixture was stirred at room temperature for 3 hours, and was partitioned between saturated aqueous sodium bicarbonate mixture and dichloromethane. The aqueous phase was extracted with dichloromethane. The combined organic phases were dried over magnesium sulfate and filtered. The filtrate was concentrated, and the residue was purified by silica gel chromatography, eluting with 5-12% methanol in dichloromethane, to provide the title compound. MS (ESI) m/z 872.3 (M+H)⁺.

### Example 42H

### (7R,20S)-18-chloro-10-{[2-(2-methoxyphenyl)pyrimidin-4-yl]methoxy}-19-methyl-15-[2-(4-methylpiperazin-1-yl)ethyl]-1-(prop-1-yn-1-yl)-7,8,15,16-tetrahydro-14H-17,20-etheno-13,9-(metheno)-6-oxa-2-thia-3,5,15-triazacyclooctadeca[1,2,3-cd]indene-7-carboxylic acid

A mixture of Example 42G (35 mg) in 0.5 mL tetrahydrofuran and 0.5 mL methanol was treated with lithium hydroxide (602 μL, 1N aqueous mixture). The mixture was stirred at room temperature overnight, adjusted to pH=6 with 1N aqueous HCl under cooling with an ice-water bath, and extracted with ethyl acetate (three times). The combined organic phases were dried over magnesium sulfate, filtered and concentrated. The residue was purified on reverse phase HPLC (5-75% acetonitrile in water with 1% trifluoroacetic acid) to provide the title compound as a trifluoroacetic acid salt, which was a mixture of two atropisomers in a ratio of 3:1 based on ¹H NMR. ¹H NMR (400 MHz, dimethyl sulfoxide-d₆) δ ppm 8.69-8.50 (m, 2H), 7.57-7.42 (m, 3H), 7.29-7.11 (m, 4H), 7.04 (t, 1H), 6.85 (d, 0.75H), 6.78 (d, 0.25H), 6.65 (d, 0.25H), 6.53 (d, 0.75H), 5.92-5.81 (m, 1H), 5.22-5.00 (m, 2H), 4.42 (m, 2H), 4.18 (m, 2H), 3.76 (s, 3H), 3.70-2.95 (m, 14H), 2.78 (s, 3H), 1.96 (s, 3H), 1.86 (s, 3H). MS (ESI) m/z 844.4 (M+H)⁺.

### Example 43

### (7R,20S)-18-chloro-1-(4-fluorophenyl)-10-{[2-(2-methoxyphenyl)pyrimidin-4-yl]methoxy}-19-methyl-15-{2-[4-(2,2,2-trifluoroethyl)piperazin-1-yl]ethyl}-7,8,15,16-tetrahydro-14H-17,20-etheno-13,9-(metheno)-6-oxa-2-thia-3,5,15-triazacyclooctadeca[1,2,3-cd]indene-7-carboxylic acid

The title compound was prepared according to the procedure described in Example 23, substituting Example 35B for 2-(4,4-difluoropiperidin-1-yl)ethanamine. ¹H NMR (500 MHz, dimethyl sulfoxide-d₆) δ ppm 8.66-8.61 (m, 2H), 7.54-7.36 (m, 3H), 7.29-7.11 (m, 7H), 7.09-6.99 (m, 2H), 6.74 (d, 1H), 6.34 (d, 1H), 5.91 (dd, 1H), 5.24-4.95 (m, 2H), 4.05-3.75 (m, 4H), 3.75 (s, 3H), 3.60 (d, 1H), 3.48-3.05 (m, 11H), 2.97-2.81 (m, 5H), 1.76 (s, 3H). MS (ESI) m/z 969 (M+H)⁺.

### Example 44

### (7R,20S)-ethyl 18-chloro-1-(4-fluorophenyl)-10-{[2-(2-methoxyphenyl)pyrimidin-4-yl]methoxy}-19-methyl-15-[2-(piperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylate

To a mixture of Example 1T (200 mg) in dichloromethane (10 mL) was added tert-butyl 4-(2-aminoethyl)piperazine-1-carboxylate (84 mg). The mixture was stirred at ambient temperature for 30 minutes, and sodium triacetoxyborohydride (104 mg) and 4 Å molecular sieves (250 mg) were added. The reaction mixture was stirred overnight and was quenched by the addition of saturated aqueous sodium bicarbonate mixture and ethyl acetate. The layers were separated, and the aqueous layer was extracted with ethyl acetate (50 mL×2). The combined organics were washed with brine, dried over anhydrous sodium sulfate, filtered and concentrated. The residue was dissolved in dichloromethane (5 mL) and trifluoroacetic acid (5 mL) was added. After 1 hour, the reaction mixture was concentrated under reduced pressure. The residue was purified by reverse phase HPLC (Zorbax C-18, 10 to 50% acetonitrile in water containing 0.1% v/v trifluoroacetic acid) to provide the title compound. ¹H NMR (400 MHz, dimethyl sulfoxide-d₆) δ ppm 9.01 (s, 1H), 8.77-8.56 (m, 2H), 7.63-7.37 (m, 3H), 7.34-7.08 (m, 8H), 7.03 (td, 1H), 6.85 (d, 1H), 6.41 (d, 1H), 5.95 (dd, 1H), 5.32-4.88 (m, 2H), 4.46-3.84 (m, 6H), 3.74 (s, 3H), 3.61-3.35 (m, 2H), 3.20 (dt, 8H), 3.04 (q, 4H), 1.75 (s, 3H), 1.00 (t, 3H). MS (ESI) m/z 915 (M+H)⁺.

### Example 45

### (7R,20S)-18-chloro-1-(4-fluorophenyl)-15-[2-(3-hydroxypyrrolidin-1-yl)ethyl]-10-{[2-(2-methoxyphenyl)pyrimidin-4-yl]methoxy}-19-methyl-7,8,15,16-tetrahydro-14H-17,20-etheno-13,9-(metheno)-6-oxa-2-thia-3,5,15-triazacyclooctadeca[1,2,3-cd]indene-7-carboxylic acid

### Example 45A

### (2R)-ethyl 2-((5-((1S)-3-chloro-4-formyl-2-methylphenyl)-6-(4-fluorophenyl)thieno[2,3-d]pyrimidin-4-yl)oxy)-3-(5-(((2-(3-hydroxypyrrolidin-1-yl)ethyl)amino)methyl)-2-((2-(2-methoxyphenyl)pyrimidin-4-yl)methoxy)phenyl)propanoate

The title compound was prepared as described in Example 41D by replacing 1-cyclopropylpiperazine with pyrrolidin-3-ol. MS (ESI) m/z 988.42 (M+H)⁺.

### Example 45B

### (7R,20S)-18-chloro-1-(4-fluorophenyl)-15-[2-(3-hydroxypyrrolidin-1-yl)ethyl]-10-{[2-(2-methoxyphenyl)pyrimidin-4-yl]methoxy}-19-methyl-7,8,15,16-tetrahydro-14H-17,20-etheno-13,9-(metheno)-6-oxa-2-thia-3,5,15-triazacyclooctadeca[1,2,3-cd]indene-7-carboxylic acid

The title compound was prepared as described in Example 34B by replacing Example 34A with Example 45A. ¹H NMR (501 MHz, dimethyl sulfoxide-d₆) δ ppm 8.71-8.58 (m, 2H), 7.57-7.36 (m, 3H), 7.28-7.12 (m, 7H), 7.10-6.96 (m, 2H), 6.73 (d, 1H), 6.38 (d, 1H), 5.92 (dd, 1H), 5.23-4.97 (m, 2H), 4.46 (h, 1H), 3.76 (s, 6H), 3.29-3.08 (m, 3H), 2.17 (s, 2H), 1.90 (dt, 1H), 1.75 (s, 3H). MS (ESI) m/z 887.3 (M+H)⁺.

### Example 46

### (7R,20S)-18-chloro-15-[2-(4-hydroxypiperidin-1-yl)ethyl]-10-{[2-(2-methoxyphenyl)pyrimidin-4-yl]methoxy}-19-methyl-1-(prop-1-yn-1-yl)-7,8,15,16-tetrahydro-14H-17,20-etheno-13,9-(metheno)-6-oxa-2-thia-3,5,15-triazacyclooctadeca[1,2,3-cd]indene-7-carboxylic acid

### Example 46A

### ethyl (7R,20S)-18-chloro-15-[2-(4-hydroxypiperidin-1-yl)ethyl]-10-{[2-(2-methoxyphenyl)pyrimidin-4-yl]methoxy}-19-methyl-1-(prop-1-yn-1-yl)-7,8,15,16-tetrahydro-14H-17,20-etheno-13,9-(metheno)-6-oxa-2-thia-3,5,15-triazacyclooctadeca[1,2,3-cd]indene-7-carboxylate

To a mixture of Example 42F (100 mg) in 13 mL dichloromethane were added 4 Å molecular sieves (50 mg), sodium triacetoxyborohydride (61.3 mg) and a mixture of 1-(2-aminoethyl)piperidin-4-ol (18.94 mg) in 1 mL dichloromethane. The mixture was stirred at room temperature overnight and partitioned between saturated aqueous sodium bicarbonate mixture and dichloromethane. The aqueous phase was extracted with dichloromethane. The combined organic phases were dried over magnesium sulfate and filtered. The filtrate was concentrated, and the residue was purified by silica gel chromatography, eluting with 30-60% methanol in dichloromethane, to provide the title compound. MS (ESI) m/z 873.4 (M+H)⁺.

### Example 46B

### (7R,20S)-18-chloro-15-[2-(4-hydroxypiperidin-1-yl)ethyl]-10-{[2-(2-methoxyphenyl)pyrimidin-4-yl]methoxy}-19-methyl-1-(prop-1-yn-1-yl)-7,8,15,16-tetrahydro-14H-17,20-etheno-13,9-(metheno)-6-oxa-2-thia-3,5,15-triazacyclooctadeca[1,2,3-cd]indene-7-carboxylic acid

A mixture of Example 46A (35 mg) in 0.5 mL tetrahydrofuran and 0.5 mL methanol was treated with LiOH (601 μL, 1N aqueous mixture). The mixture was stirred at room temperature overnight. The mixture was diluted with 10 mL water, and the pH was adjusted to about 5-6 with acetic acid. The mixture was extracted with ethyl acetate (3×60 mL), washed with brine, dried over magnesium sulfate and filtered. The filtrate was concentrated. The residue was taken up in 2 mL N,N-dimethylformamide and purified by reverse phase HPLC (5-75% acetonitrile in water with 1% trifluoroacetic acid to provide the title compound and Example 47 as separable atropisomers. ¹H NMR (400 MHz, dimethyl sulfoxide-d₆) δ ppm 8.65 (s, 1H), 8.59 (d, 1H), 7.53-7.42 (m, 4H), 7.22-7.11 (m, 3H), 7.08-6.99 (m, 2H), 6.74 (d, 1H), 6.37 (s, 1H), 5.84 (dd, 1H), 5.18-4.96 (m, 2H), 3.95 (d, 1H), 3.76 (s, 3H), 3.82-3.0 (m, 16H), 1.97 (s, 3H), 1.90 (s, 3H). LC/MS (ESI) m/z 845.6 (M+H)⁺.

### Example 47

### (7R,20R)-18-chloro-15-[2-(4-hydroxypiperidin-1-yl)ethyl]-10-{[2-(2-methoxyphenyl)pyrimidin-4-yl]methoxy}-19-methyl-1-(prop-1-yn-1-yl)-7,8,15,16-tetrahydro-14H-17,20-etheno-13,9-(metheno)-6-oxa-2-thia-3,5,15-triazacyclooctadeca[1,2,3-cd]indene-7-carboxylic acid

The title compound was isolated during the synthesis of Example 46B. ¹H NMR (400 MHz, dimethyl sulfoxide-d₆) δ ppm 8.60 (s, 1H), 8.55 (d, 1H), 7.52-7.41 (m, 3H), 7.23 (d, 1H), 7.13 (d, 1H), 7.03 (dt, 3H), 6.91 (d, 1H), 6.76 (t, 2H), 6.56 (s, 1H), 5.80 (dd, 1H), 5.13 (s, 2H), 4.22 (d, 1H), 3.85-3.02 (m, 16H), 3.73 (s, 3H), 2.27 (s, 3H), 1.96 (s, 3H). LC/MS (ESI) m/z 845.6 (M+H)⁺.

### Example 48

### (7R,20S)-18-chloro-1-(4-fluorophenyl)-10-{[2-(2-methoxyphenyl)pyrimidin-4-yl]methoxy}-19-methyl-15-[2-(1-methylpiperidin-4-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylic acid

The title compound was prepared according to the procedure described in Example 23, substituting 2-(1-methylpiperidin-4-yl)ethanamine for 2-(4,4-difluoropiperidin-1-yl)ethanamine. ¹H NMR (500 MHz, dimethyl sulfoxide-d₆) δ ppm 8.63 (d, 2H), 7.71-7.38 (m, 3H), 7.40-7.10 (m, 9H), 7.04 (t, 1H), 6.87 (s, 1H), 6.63 (s, 1H), 5.98 (s, 1H), 5.31-4.96 (m, 2H), 4.69-4.15 (m, 3H), 3.75 (s, 3H), 3.74-3.62 (m, 4H), 3.52-3.06 (m, 4H), 3.00-2.68 (m, 5H), 2.04-1.81 (m, 4H), 1.70 (s, 3H), 1.44 (t, 2H). MS (ESI) m/z 900 (M+H)⁺.

### Example 49

### (7R,16R,21R)-19-chloro-1-(4-fluorophenyl)-10-{[2-(2-methoxyphenyl)pyrimidin-4-yl]methoxy}-20-methyl-16-[(4-methylpiperazin-1-yl)methyl]-7,8,15,16-tetrahydro-18,21-etheno-9,13-(metheno)-2,6,14,17-tetraoxa-3,5-diazacyclononadeca[1,2,3-cd]indene-7-carboxylic acid

### Example 49A

### 4-chloro-6-iodofuro[2,3-d]pyrimidine

To a mixture of 4-chlorofuro[2,3-d]pyrimidine (1 g) in tetrahydrofuran (30.8 mL) at −78° C. was added lithium diisopropylamide (1 M in tetrahydrofuran/hexane, 7.1 mL) over ˜5 minutes, and the mixture was allowed to stir at −78° C. for 1 hour. A mixture of iodine (1.8 g) in tetrahydrofuran (15.4 mL) was added over 10 minutes, and the reaction mixture was allowed to stir. The cooling bath was removed after 15 minutes, and the reaction mixture was stirred at room temperature overnight. The reaction mixture was quenched with 10% sodium thiosulfate mixture, cooled to 0° C., and stirred for 1 hour. The mixture was filtered, and the material was washed with water and pentane and dried under vacuum to provide the title compound. MS(ESI) m/z 281.0 (M+H)⁺.

### Example 49B

### 4-chloro-6-(4-fluorophenyl)furo[2,3-d]pyrimidine

Two 20 mL microwave vials were charged with Example 49A (770 mg), (4-fluorophenyl)boronic acid (500 mg), tris(dibenzylideneacetone)dipalladium (50 mg) and 2-di-tert-butylphosphino-2′-4′-6′-triisopropylbiphenyl (47 mg) and purged with nitrogen for 30 minutes. Tetrahydrofuran (8.8 mL) and water (2.2 mL) were purged with nitrogen and added to the vials. Each vial was heated under microwave irradiation (Biotage® Initiator) for 2 hours at 80° C. The reactions were cooled, combined, diluted with dichloromethane, washed with water twice and washed with brine. The organic layer was dried over sodium sulfate, filtered and concentrated. The residue was purified by normal phase MPLC on a Teledyne Isco Combiflash Rf+ (0-20% ethyl acetate in heptanes) to provide the title compound. MS(ESI) m/z 249.3 (M+H)⁺.

### Example 49C

### 5-bromo-4-chloro-6-(4-fluorophenyl)furo[2,3-d]pyrimidine

To a mixture of Example 49B (1.2 g) in N,N-dimethylformamide (23.5 mL) at room temperature was added N-bromosuccinimide (1.2 g), and the reaction mixture was allowed to stir overnight. The reaction mixture was diluted with water and extracted with dichloromethane (3 times). The combined organic extracts were washed with water and brine, dried over sodium sulfate, filtered and concentrated. The residue was purified by normal phase MPLC on a Teledyne Isco Combiflash Rf+ (0-15% ethyl acetate in heptanes) to provide the title compound. MS(ESI) m/z 329.0 (M+H)⁺.

### Example 49D

### (R)-ethyl 2-((5-bromo-6-(4-fluorophenyl)furo[2,3-d]pyrimidin-4-yl)oxy)-3-(5-((tert-butyldimethylsilyl)oxy)-2-((2-(2-methoxyphenyl)pyrimidin-4-yl)methoxy)phenyl)propanoate

To a mixture of Example 49C (200 mg) and Example 68B (330 mg) in tert-butanol (6.1 mL) was added cesium carbonate (600 mg), and the reaction mixture was heated at 65° C. for 4 hours. After cooling, some tert-butanol was removed under vacuum, and the mixture was diluted with water and brine. The mixture was extracted with ethyl acetate (three times), and the combined organic layers were dried over sodium sulfate, filtered and concentrated. The residue was purified by normal phase MPLC on a Teledyne Isco Combiflash Rf+ (5-60% ethyl acetate in heptanes) to provide the title compound. MS (ESI) m/z 829.2 (M+H)⁺.

### Example 49E

### (2R)-ethyl 2-((5-(4-(((R)-1-(bis(4-methoxyphenyl)(phenyl)methoxy)-3-(4-methylpiperazin-1-yl)propan-2-yl)oxy)-3-chloro-2-methylphenyl)-6-(4-fluorophenyl)furo[2,3-d]pyrimidin-4-yl)oxy)-3-(5-((tert-butyldimethylsilyl)oxy)-2-((2-(2-methoxyphenyl)pyrimidin-4-yl)methoxy)phenyl)propanoate

To a vial containing Example 49D (200 mg), Example 64K (230 mg), cesium carbonate (240 mg) and bis(di-tert-butyl(4-dimethylaminophenyl)phosphine)dichloropalladium (17 mg) was added degassed tetrahydrofuran (2.4 mL) and water (600 μL), and the reaction mixture was allowed to stir at room temperature for 3 days. To the reaction mixture was added 1-pyrrolidinecarboditioic acid ammonium salt (4 mg), and the mixture was stirred for 30 minutes. The reaction mixture was filtered over diatomaceous earth, washing with ethyl acetate. The filtrate was diluted with water and brine and extracted with ethyl acetate (three times). The combined organic layers were dried over sodium sulfate, filtered and concentrated. The residue was purified by normal phase MPLC on a Teledyne Isco Combiflash Rf+ (0-6% methanol in dichloromethane) to provide the title compound. MS (ESI) m/z 1350.5 (M+H)⁺.

### Example 49F

### (2R)-ethyl 3-(5-((tert-butyldimethylsilyl)oxy)-2-((2-(2-methoxyphenyl)pyrimidin-4-yl)methoxy)phenyl)-2-((5-(3-chloro-4-(((R)-1-hydroxy-3-(4-methylpiperazin-1-yl)propan-2-yl)oxy)-2-methylphenyl)-6-(4-fluorophenyl)furo[2,3-d]pyrimidin-4-yl)oxy)propanoate

To a mixture of Example 49E (150 mg) in dichloromethane (600 μL) and methanol (600 μL) was added formic acid (630 μL), and the reaction mixture was allowed to stir for 90 minutes. The reaction mixture was slowly quenched with saturated sodium bicarbonate mixture and was extracted with ethyl acetate (three times). The combined organic extracts were washed with brine, dried over sodium sulfate, filtered and concentrated to provide the title compound which was used without further purification. MS (ESI) m/z 1047.3 (M+H)⁺.

### Example 49G

### (2R)-ethyl 2-((5-(3-chloro-4-(((R)-1-hydroxy-3-(4-methylpiperazin-1-yl)propan-2-yl)oxy)-2-methylphenyl)-6-(4-fluorophenyl)furo[2,3-d]pyrimidin-4-yl)oxy)-3-(5-hydroxy-2-((2-(2-methoxyphenyl)pyrimidin-4-yl)methoxy)phenyl)propanoate

To a mixture of Example 49F (114 mg) in tetrahydrofuran (1 mL) at room temperature was added tetrabutyl ammonium fluoride (1 M in tetrahydrofuran, 330 μL), and the reaction mixture was allowed to stir for 40 minutes. The reaction mixture was quenched with saturated ammonium chloride and extracted with ethyl acetate (three times). The combined organic layers were washed with water, dried over sodium sulfate, filtered and concentrated. The crude residue was purified by normal phase MPLC on a Teledyne Isco Combiflash® Rf+ (1-10% methanol in dichloromethane) followed by reverse-phase HPLC on a Gilson PLC 2020 using a Luna column (250×50 mm, 10 m) (5-75% acetonitrile in water containing 0.1% trifluoroacetic acid). The product containing fractions were combined and neutralized with saturated sodium bicarbonate. The mixture was extracted with dichloromethane (three times), and the combined organic layers were dried over sodium sulfate, filtered and concentrated to provide the title compound as a mixture of atropisomers containing an unknown amount of tetrabutyl ammonium salt. MS (ESI) m/z 933.4 (M+H)⁺.

### Example 49H

### ethyl (7R,16R)-19-chloro-1-(4-fluorophenyl)-10-{[2-(2-methoxyphenyl)pyrimidin-4-yl]methoxy}-20-methyl-16-[(4-methylpiperazin-1-yl)methyl]-7,8,15,16-tetrahydro-18,21-etheno-9,13-(metheno)-2,6,14,17-tetraoxa-3,5-diazacyclononadeca[1,2,3-cd]indene-7-carboxylate

To a mixture of Example 49G (57 mg) in toluene (6.1 mL) was added triphenylphosphine (48 mg) followed by N,N,N′N′-tetramethylazodicaboxamide (32 mg), and the reaction mixture was allowed to stir overnight. The reaction mixture was diluted with ethyl acetate, filtered over diatomaceous earth and concentrated. The residue was purified by reverse-phase HPLC on a Gilson PLC 2020 using a Luna column (250×50 mm, 10 m) (5-70% acetonitrile in water containing 0.1% trifluoroacetic acid) to provide the title compound. MS (ESI) m/z 915.4 (M+H)⁺.

### Example 49I

### (7R,16R,21R)-19-chloro-1-(4-fluorophenyl)-10-{[2-(2-methoxyphenyl)pyrimidin-4-yl]methoxy}-20-methyl-16-[(4-methylpiperazin-1-yl)methyl]-7,8,15,16-tetrahydro-18,21-etheno-9,13-(metheno)-2,6,14,17-tetraoxa-3,5-diazacyclononadeca[1,2,3-cd]indene-7-carboxylic acid

To a mixture of Example 49H (39 mg) in tetrahydrofuran (375 μL) and methanol (375 μL) was added a mixture of lithium hydroxide (16 mg) in water (375 μL), and the reaction mixture was allowed to stir overnight. The reaction mixture was quenched with trifluoroacetic acid (65 μL) and was purified by reverse-phase HPLC on a Gilson PLC 2020 using a Luna column (250×50 mm, 10 m) (5-65% acetonitrile in water containing 0.1% trifluoroacetic acid) to provide the title compound. ¹H NMR (400 MHz, dimethyl sulfoxide-d₆) δ ppm 8.85 (d, 1H), 8.51 (s, 1H), 7.59 (d, 1H), 7.57-7.40 (m, 4H), 7.30-7.17 (m, 3H), 7.13 (d, 1H), 7.03 (t, 1H), 6.95 (d, 1H), 6.85 (d, 1H), 6.77 (dd, 1H), 6.11 (d, 1H), 5.61 (dd, 1H), 5.25-5.08 (m, 3H), 4.32-4.24 (m, 1H), 4.13 (dd, 1H), 3.74 (s, 3H), 3.08-2.90 (m, 2H), 2.81 (s, 3H), 2.76-2.63 (m, 1H), 2.43 (s, 3H). MS (ESI) m/z 887.3 (M+H)⁺.

### Example 50

### (7R,20S)-18-chloro-1-(4-fluorophenyl)-10-{[2-(2-methoxyphenyl)pyrimidin-4-yl]methoxy}-19-methyl-15-[3-(4-methylpiperazin-1-yl)propyl]-7,8,15,16-tetrahydro-14H-17,20-etheno-13,9-(metheno)-6-oxa-2-thia-3,5,15-triazacyclooctadeca[1,2,3-cd]indene-7-carboxylic acid

To a mixture of Example 1T (65 mg) in dichloromethane (2 mL) was added 3-(4-methylpiperazin-1-yl)propan-1-amine (24 mg). The mixture was stirred for 20 minutes at room temperature before the addition of sodium triacetoxyborohydride (33 mg). The mixture was stirred at room temperature for 40 minutes. The reaction mixture was diluted with ethyl acetate (200 mL) and washed with water and brine and dried over sodium sulfate. Evaporation of the solvent gave the crude product, which was dissolved in dichloromethane (8 mL), trifluoroacetic acid (2 mL) and a few drops of water. The mixture was stirred at room temperature for 4 hours. The mixture was concentrated under vacuum. The residue was dissolved in ethyl acetate (200 mL) and washed with saturated aqueous sodium bicarbonate mixture (50 mL) and brine and dried over sodium sulfate. Filtration and evaporation of the solvent gave a residue that was dissolved in tetrahydrofuran (5 mL). Decaborane (30 mg) was added, and the mixture was stirred at room temperature for 10 minutes. The reaction mixture was added to a mixture of methanol (10 mL) and 1N aqueous HCl (30 mL) and was stirred at room temperature for 2 hours. The reaction mixture was basified with solid K₂CO₃, diluted with ethyl acetate (200 mL), washed with saturated aqueous sodium bicarbonate mixture and brine, and dried over sodium sulfate. Filtration and evaporation of the solvent gave a residue that was dissolved in tetrahydrofuran (4 mL), methanol (2 mL) and water (2 mL). Lithium hydroxide monohydrate (50 mg) was added, and the mixture was stirred at room temperature for 3 hours. LC/MS showed the saponification was complete, and the mixture was acidified with trifluoroacetic acid and concentrated under vacuum. The residue was dissolved in N,N-dimethylformamide (8 mL) and was purified by reverse-phase chromatography on a Gilson HPLC (Phenomenex®, 250×50 mm, C18 column), eluting with 20 to 80% acetonitrile in water (0.1% trifluoroacetic acid) over 35 minutes to provide the title compound. ¹H NMR (501 MHz, dimethyl sulfoxide-d₆) δ ppm 8.64 (q, 2H), 7.57-7.43 (m, 3H), 7.30 (d, 1H), 7.28-7.21 (m, 3H), 7.19-7.11 (m, 4H), 7.05 (t, 1H), 6.86 (d, 1H), 6.56 (d, 1H), 5.95 (dd, 1H), 5.23-4.88 (m, 2H), 4.43-4.02 (m, 4H), 3.76 (s, 3H), 3.29-3.10 (m, 2H), 2.79 (s, 3H), 2.71 (s, 2H), 2.10 (s, 2H), 1.71 (s, 3H). MS (ESI) m/z 914.3 (M+H)⁺.

### Example 51

### (7R,21S)-19-chloro-16-[2-(4,4-difluoropiperidin-1-yl)ethyl]-1-(4-fluorophenyl)-20-methyl-15-oxo-10-{[2-(3,3,3-trifluoropropoxy)pyrimidin-4-yl]methoxy}-7,8,14,15,16,17-hexahydro-18,21-etheno-13,9-(metheno)-6-oxa-2-thia-3,5,16-triazacyclononadeca[1,2,3-cd]indene-7-carboxylic acid

### Example 51A

### ethyl (R)-3-(5-(2-(tert-butoxy)-2-oxoethyl)-2-((2-(3,3,3-trifluoropropoxy)pyrimidin-4-yl)methoxy)phenyl)-2-((5-((1S)-3-chloro-4-(((2-(4,4-difluoropiperidin-1-yl)ethyl)amino)methyl)-2-methylphenyl)-6-(4-fluorophenyl)thieno[2,3-d]pyrimidin-4-yl)oxy)propanoate

The title compound was made according to the procedure described for Example 35E, substituting 2-(4,4-difluoropiperidin-1-yl)ethan-1-amine for Example 35B. MS (APCI) m/z 1057.42 (M)⁺.

### Example 51B

### (R)-2-(3-(2-((5-((1S)-3-chloro-4-(((2-(4,4-difluoropiperidin-1-yl)ethyl)amino)methyl)-2-methylphenyl)-6-(4-fluorophenyl)thieno[2,3-d]pyrimidin-4-yl)oxy)-3-ethoxy-3-oxopropyl)-4-((2-(3,3,3-trifluoropropoxy)pyrimidin-4-yl)methoxy)phenyl)acetic acid

The title compound was made according to the procedure described for Example 35F, substituting Example 51A for Example 35E.

### Example 51C

### ethyl (7R,21S)-19-chloro-16-[2-(4,4-difluoropiperidin-1-yl)ethyl]-1-(4-fluorophenyl)-20-methyl-15-oxo-10-{[2-(3,3,3-trifluoropropoxy)pyrimidin-4-yl]methoxy}-7,8,14,15,16,17-hexahydro-18,21-etheno-13,9-(metheno)-6-oxa-2-thia-3,5,16-triazacyclononadeca[1,2,3-cd]indene-7-carboxylate

The title compound was synthesized according to the procedure described for 35G, substituting Example 51B for Example 35F. MS (APCI) m/z 1001.2 (M+H)⁺.

### Example 51D

### (7R,21S)-19-chloro-16-[2-(4,4-difluoropiperidin-1-yl)ethyl]-1-(4-fluorophenyl)-20-methyl-15-oxo-10-{[2-(3,3,3-trifluoropropoxy)pyrimidin-4-yl]methoxy}-7,8,14,15,16,17-hexahydro-18,21-etheno-13,9-(metheno)-6-oxa-2-thia-3,5,16-triazacyclononadeca[1,2,3-cd]indene-7-carboxylic acid

The title compound was synthesized according to the procedure described for 35H, substituting Example 51C for Example 35G. ¹H NMR (501 MHz, dimethyl sulfoxide-d₆) δ ppm 9.68 (s, 1H), 8.49 (s, 1H), 8.46 (d, 1H), 7.27 (t, 2H), 7.16 (t, 2H), 7.04 (dd, 1H), 6.86-6.76 (m, 1H), 6.73 (d, 1H), 6.69-6.54 (m, 2H), 4.91 (d, 1H), 4.66 (d, 1H), 4.55-4.40 (m, 5H), 3.88 (d,), 3.70-3.02 (m, 13H), 2.82 (qt, 2H), 2.44-2.21 (m, 2H), 1.86 (s, 3H). MS (ESI) m/z 955.2 (M+H)⁺.

### Example 52

### (7R,20S)-18-chloro-1-(4-fluorophenyl)-15-{3-[4-(2-hydroxyethyl)piperazin-1-yl]propyl}-10-{[2-(2-methoxyphenyl)pyrimidin-4-yl]methoxy}-19-methyl-7,8,15,16-tetrahydro-14H-17,20-etheno-13,9-(metheno)-6-oxa-2-thia-3,5,15-triazacyclooctadeca[1,2,3-cd]indene-7-carboxylic acid

The title compound was prepared as described in Example 50 by replacing 3-(4-methylpiperazin-1-yl)propan-1-amine with 2-(4-(3-aminopropyl)piperazin-1-yl)ethanol. ¹H NMR (400 MHz, dimethyl sulfoxide-d₆) δ 8.73-8.61 (m, 2H), 7.56-7.45 (m, 4H), 7.35-7.12 (m, 12H), 7.05 (t, 1H), 6.86 (d, 1H), 6.56 (d, 1H), 5.95 (dd, 1H), 5.27-4.99 (m, 2H), 4.49-4.10 (m, 6H), 3.75 (d, 6H), 3.24-3.04 (m, 6H), 2.79 (d, 3H), 2.12 (dd, 3H), 1.72 (s, 3H). MS (ESI) m/z 944.2 (M+H)⁺.

### Example 53

### (7R,21R)-19-chloro-16-[2-(4,4-difluoropiperidin-1-yl)ethyl]-1-(4-fluorophenyl)-20-methyl-15-oxo-10-{[2-(3,3,3-trifluoropropoxy)pyrimidin-4-yl]methoxy}-7,8,14,15,16,17-hexahydro-18,21-etheno-13,9-(metheno)-6-oxa-2-thia-3,5,16-triazacyclononadeca[1,2,3-cd]indene-7-carboxylic acid

The title compound was isolated as a minor component during the synthesis of Example 51D. ¹H NMR (501 MHz, dimethyl sulfoxide-d₆) δ ppm 9.56 (s, 1H), 8.50 (s, 1H), 8.28 (s, 1H), 7.30-7.22 (m, 2H), 7.19-7.11 (m, 2H), 7.03 (dd, 1H), 6.75 (d, 2H), 6.50 (d, 1H), 6.05 (d, 1H), 5.14 (s, 1H), 4.99 (d, 1H), 4.78 (d, 1H), 4.58 (d, 1H), 4.52-4.43 (m, 2H), 4.36 (s, 1H), 3.97 (s, 1H), 3.88-3.00 (m, 15H), 2.80 (qt, 2H), 2.31 (s, 3H). MS (ESI) m/z 955.2 (M+H)⁺.

### Example 54

### (7R,21S)-19-chloro-1-(4-fluorophenyl)-16-{2-[4-(methanesulfonyl)piperazin-1-yl]ethyl}-20-methyl-15-oxo-10-{[2-(3,3,3-trifluoropropoxy)pyrimidin-4-yl]methoxy}-7,8,14,15,16,17-hexahydro-18,21-etheno-13,9-(metheno)-6-oxa-2-thia-3,5,16-triazacyclononadeca[1,2,3-cd]indene-7-carboxylic acid

### Example 54A

### ethyl (R)-3-(5-(2-(tert-butoxy)-2-oxoethyl)-2-((2-(3,3,3-trifluoropropoxy)pyrimidin-4-yl)methoxy)phenyl)-2-((5-((1S)-3-chloro-2-methyl-4-(((2-(4-(methylsulfonyl)piperazin-1-yl)ethyl)amino)methyl)phenyl)-6-(4-fluorophenyl)thieno[2,3-d]pyrimidin-4-yl)oxy)propanoate

The title compound was made according to the procedure described for Example 35E, substituting 2-(4-(methylsulfonyl)piperazin-1-yl)ethan-1-amine for Example 35B. MS (APCI) m/z 1100.5 (M+H)⁺.

### Example 54B

### (R)-2-(3-(2-((5-((1S)-3-chloro-2-methyl-4-(((2-(4-(methylsulfonyl)piperazin-1-yl)ethyl)amino)methyl)phenyl)-6-(4-fluorophenyl)thieno[2,3-d]pyrimidin-4-yl)oxy)-3-ethoxy-3-oxopropyl)-4-((2-(3,3,3-trifluoropropoxy)pyrimidin-4-yl)methoxy)phenyl)acetic acid

The title compound was prepared as described in Example 35F, substituting Example 54A for Example 35E. MS (APCI) m/z 1044.2 (M+H)⁺.

### Example 54C

### ethyl (7R,21S)-19-chloro-1-(4-fluorophenyl)-16-{2-[4-(methanesulfonyl)piperazin-1-yl]ethyl}-20-methyl-15-oxo-10-{[2-(3,3,3-trifluoropropoxy)pyrimidin-4-yl]methoxy}-7,8,14,15,16,17-hexahydro-18,21-etheno-13,9-(metheno)-6-oxa-2-thia-3,5,16-triazacyclononadeca[1,2,3-cd]indene-7-carboxylate

The title compound was prepared as described in Example 35G, substituting Example 54B for Example 35F. MS (APCI) m/z 1026.2 (M+H)⁺.

### Example 54D

### (7R,21S)-19-chloro-1-(4-fluorophenyl)-16-{2-[4-(methanesulfonyl)piperazin-1-yl]ethyl}-20-methyl-15-oxo-10-{[2-(3,3,3-trifluoropropoxy)pyrimidin-4-yl]methoxy}-7,8,14,15,16,17-hexahydro-18,21-etheno-13,9-(metheno)-6-oxa-2-thia-3,5,16-triazacyclononadeca[1,2,3-cd]indene-7-carboxylic acid

The title compound was synthesized according to the procedure described for 35H, substituting Example 54C for Example 35G. ¹H NMR (501 MHz, dimethyl sulfoxide-d₆) δ ppm 9.52 (s, 1H), 8.50 (s, 1H), 8.47 (d, 1H), 7.26 (d, 2H), 7.16 (t, 2H), 7.04 (dd, 1H), 6.83 (s, 1H), 6.73 (d, 1H), 6.71-6.48 (m, 2H), 4.90 (d, 1H), 4.66 (d, 1H), 4.48 (qp, 5H), 3.88 (d, 1H), 3.60-3.36 (m, 15H), 3.04 (s, 3H), 2.82 (qt, 2H), 1.88 (s, 3H). MS (ESI) m/z 998.3 (M+H)⁺.

### Example 55

### (7R,21S)-19-chloro-1-(4-fluorophenyl)-20-methyl-15-oxo-16-[2-(3-oxopiperazin-1-yl)ethyl]-10-{[2-(3,3,3-trifluoropropoxy)pyrimidin-4-yl]methoxy}-7,8,14,15,16,17-hexahydro-18,21-etheno-13,9-(metheno)-6-oxa-2-thia-3,5,16-triazacyclononadeca[1,2,3-cd]indene-7-carboxylic acid

### Example 55A

### ethyl (R)-3-(5-(2-(tert-butoxy)-2-oxoethyl)-2-((2-(3,3,3-trifluoropropoxy)pyrimidin-4-yl)methoxy)phenyl)-2-((5-((1S)-3-chloro-2-methyl-4-(((2-(3-oxopiperazin-1-yl)ethyl)amino)methyl)phenyl)-6-(4-fluorophenyl)thieno[2,3-d]pyrimidin-4-yl)oxy)propanoate

The title compound was prepared as described in Example 35E, substituting 4-(2-aminoethyl)piperazin-2-one for Example 35B. MS (APCI) m/z 1036.3 (M+H)⁺.

### Example 55B

### (R)-2-(3-(2-((5-((1S)-3-chloro-2-methyl-4-(((2-(3-oxopiperazin-1-yl)ethyl)amino)methyl)phenyl)-6-(4-fluorophenyl)thieno[2,3-d]pyrimidin-4-yl)oxy)-3-ethoxy-3-oxopropyl)-4-((2-(3,3,3-trifluoropropoxy)pyrimidin-4-yl)methoxy)phenyl)acetic acid

The title compound prepared as described in Example 35F, substituting Example 55A for Example 35E. MS (APCI) m/z 980.2 (M+H)⁺.

### Example 55C

### ethyl (7R,21S)-19-chloro-1-(4-fluorophenyl)-20-methyl-15-oxo-16-[2-(3-oxopiperazin-1-yl)ethyl]-10-{[2-(3,3,3-trifluoropropoxy)pyrimidin-4-yl]methoxy}-7,8,14,15,16,17-hexahydro-18,21-etheno-13,9-(metheno)-6-oxa-2-thia-3,5,16-triazacyclononadeca[1,2,3-cd]indene-7-carboxylate

The title compound was prepared as described in Example 35G, substituting Example 55B for Example 35F. MS (APCI) m/z 962.01 (M+H)⁺.

### Example 55D

### (7R,21S)-19-chloro-1-(4-fluorophenyl)-20-methyl-15-oxo-16-[2-(3-oxopiperazin-1-yl)ethyl]-10-{[2-(3,3,3-trifluoropropoxy)pyrimidin-4-yl]methoxy}-7,8,14,15,16,17-hexahydro-18,21-etheno-13,9-(metheno)-6-oxa-2-thia-3,5,16-triazacyclononadeca[1,2,3-cd]indene-7-carboxylic acid

The title compound was prepared as described in Example 35H, substituting Example 55C for Example 35G. ¹H NMR (501 MHz, dimethyl sulfoxide-d₆) δ ppm 8.50 (s, 1H), 8.48-8.44 (m, 2H), 7.62 (s, 1H), 7.26 (q, 2H), 7.21-7.13 (m, 2H), 7.04 (td, 1H), 6.69-6.40 (m, 2H), 6.83 (s, 1H), 6.72 (dd, 1H), 4.90 (d, 1H), 4.67 (d, 1H), 4.56-4.35 (m, 4H), 3.97-3.77 (m, 2H), 3.68-2.97 (m, 12H), 2.96-2.86 (m, 2H), 2.81 (ddt, 2H), 1.85 (s, 3H). MS (ESI) m/z 934.2 (M+H)⁺.

### Example 56

### (7R,20S)-18-chloro-1-(4-fluorophenyl)-10-{[2-(2-methoxyphenyl)pyrimidin-4-yl]methoxy}-19-methyl-15-{2-[4-(methylamino)piperidin-1-yl]ethyl}-7,8,15,16-tetrahydro-14H-17,20-etheno-13,9-(metheno)-6-oxa-2-thia-3,5,15-triazacyclooctadeca[1,2,3-cd]indene-7-carboxylic acid

### Example 56A

### tert-butyl (1-(2-(((benzyloxy)carbonyl)amino)ethyl)piperidin-4-yl)(methyl)carbamate

To a mixture of benzyl (2-bromoethyl)carbamate (500 mg) in N,N-dimethylformamide (5 mL) was added triethylamine and tert-butyl methyl(piperidin-4-yl)carbamate (623 mg). The mixture was heated to 50° C. overnight. Thin layer chromatography showed the starting material was consumed. The reaction mixture was quenched with sodium bicarbonate mixture and was extracted with ethyl acetate (2×50 mL). The organic phase was concentrated and was purified by silica gel chromatography on a CombiFlash® Teledyne Isco system eluting with 100% ethyl acetate to provide the title compound. LC/MS (ESI) m/z 392 (M+H)⁺.

### Example 56B

### tert-butyl (1-(2-aminoethyl)piperidin-4-yl)(methyl)carbamate

To a mixture of Example 56A (160 mg) in methanol (5 mL) was added Pd/C (10%, 40 mg). The mixture was degassed and filled with H₂ and stirred at room temperature overnight under H₂. Thin layer chromatography showed the starting material was consumed. The reaction mixture was filtered and concentrated to give a residue, which was used in the next step without purification. LC/MS (ESI) m/z 258 (M+H)⁺.

### Example 56C

### (7R,20S)-18-chloro-1-(4-fluorophenyl)-10-{[2-(2-methoxyphenyl)pyrimidin-4-yl]methoxy}-19-methyl-15-{2-[4-(methylamino)piperidin-1-yl]ethyl}-7,8,15,16-tetrahydro-14H-17,20-etheno-13,9-(metheno)-6-oxa-2-thia-3,5,15-triazacyclooctadeca[1,2,3-cd]indene-7-carboxylic acid

To a mixture of Example 1U (50 mg) in dichloromethane (5 mL) and acetic acid (1 mL) was added Example 56B (23 mg). Molecular sieves (4 Å, 50 mg) were added. The mixture was stirred at room temperature for 1 hour before the addition of sodium triacetoxyborohydride (26 mg). The mixture was stirred at room temperature overnight. The reaction mixture was quenched by the addition of saturated aqueous sodium bicarbonate. The reaction mixture was extracted with ethyl acetate (50 mL×2). The combined organic phases were washed with brine and dried over sodium sulfate. The mixture was filtered, and the solvent was removed to give a crude product, which was dissolved in dichloromethane (2 mL) and trifluoroacetic acid (0.5 mL). The mixture was stirred for 30 minutes, quenched with water, and partitioned between water and ethyl acetate. The organic phase was concentrated. The residue was dissolved in a mixture of tetrahydrofuran (2 mL), water (1 mL) and methanol (1 mL). Lithium hydroxide (5 mg) was added. The reaction mixture was stirred at room temperature overnight. The mixture was acidified with trifluoroacetic acid and concentrated. The residue was purified by reverse-phase chromatography on a Gilson HPLC (Phenomenex®, 250×50 mm, C18 column), eluting with 20-80% acetonitrile in water (0.1% trifluoroacetic acid) over 35 minutes to provide the title compound. ¹H NMR (400 MHz, dimethyl sulfoxide-d₆) δ ppm 8.92-8.73 (m, 1H), 8.65-8.52 (m, 2H), 7.61-7.31 (m, 3H), 7.34-7.07 (m, 8H), 7.05-6.91 (m, 2H), 6.70 (d, 1H), 6.33 (d, 1H), 5.88 (dd, 1H), 5.23-4.94 (m, 2H), 3.81 (d, 1H), 3.72 (s, 3H), 3.49 (s, 7H), 3.13 (dtd, 6H), 2.62-2.49 (m, 4H), 2.19 (d, 2H), 1.83-1.71 (m, 2H), 1.71 (s, 3H). MS (ESI) m/z 915 (M+H)⁺.

### Example 57

### (7R,20S)-18-chloro-15-{2-[4-(dimethylamino)piperidin-1-yl]ethyl}-1-(4-fluorophenyl)-10-{[2-(2-methoxyphenyl)pyrimidin-4-yl]methoxy}-19-methyl-7,8,15,16-tetrahydro-14H-17,20-etheno-13,9-(metheno)-6-oxa-2-thia-3,5,15-triazacyclooctadeca[1,2,3-cd]indene-7-carboxylic acid

### Example 57A

### benzyl (2-(4-(dimethylamino)piperidin-1-yl)ethyl)carbamate

A mixture of N,N-dimethylpiperidin-4-amine (217 mg) in dichloromethane (5 mL) and acetic acid (0.5 mL) was added tert-butyl (2-oxoethyl)carbamate (300 mg) followed by addition of sodium triacetoxyborohydride (658 mg). The mixture was stirred at room temperature overnight. The reaction mixture was quenched with saturated aqueous sodium bicarbonate mixture, and was extracted with ethyl acetate (2×50 mL). The organic phase was concentrated and the crude material was purified by silica gel chromatography on a CombiFlash® Teledyne Isco system eluting with 100% ethyl acetate to provide the title compound. LC/MS (ESI) m/z 306 (M+H)⁺.

### Example 57B

### 1-(2-aminoethyl)-N,N-dimethylpiperidin-4-amine

To a mixture of Example 57A (150 mg) in methanol (5 mL) was added Pd/C (10%, 40 mg). The mixture was degassed, filled with H₂ and stirred at room temperature overnight under H₂. Thin layer chromatography showed the starting material was consumed. The reaction mixture was filtered and concentrated to provide the title compound, which was used in the next step without further purification. LC/MS (ESI) m/z 171 (M+H)⁺.

### Example 57C

### (7R,20S)-18-chloro-15-{2-[4-(dimethylamino)piperidin-1-yl]ethyl}-1-(4-fluorophenyl)-10-{[2-(2-methoxyphenyl)pyrimidin-4-yl]methoxy}-19-methyl-7,8,15,16-tetrahydro-14H-17,20-etheno-13,9-(metheno)-6-oxa-2-thia-3,5,15-triazacyclooctadeca[1,2,3-cd]indene-7-carboxylic acid

Example 57C was prepared as described in Example 23, substituting Example 57B for 2-(4,4-difluoropiperidin-1-yl)ethanamine. ¹H NMR (400 MHz, dimethyl sulfoxide-d₆) δ ppm 8.71-8.45 (m, 2H), 7.55-7.29 (m, 3H), 7.29-7.06 (m, 8H), 7.06-6.90 (m, 2H), 6.71 (d, 1H), 6.33 (d, 1H), 5.89 (dd, 1H), 5.22-4.90 (m, 2H), 3.93-3.73 (m, 8H), 3.72 (s, 3H), 3.38 (t, 2H), 3.30-2.95 (m, 5H), 2.77 (s, 6H), 2.22 (d, 2H), 1.95-1.77 (m, 2H), 1.71 (s, 3H). MS (ESI) m/z 929 (M+H)⁺.

### Example 58

### (7R,20S)-18-chloro-1-(4-fluorophenyl)-10-{[2-(2-methoxyphenyl)pyrimidin-4-yl]methoxy}-19-methyl-15-[2-(4-methyl-3-oxopiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylic acid

### Example 58A

### benzyl (2-(4-methyl-3-oxopiperazin-1-yl)ethyl)carbamate

To a mixture of benzyl (2-bromoethyl)carbamate (500 mg) in N,N-dimethylformamide (5 mL) was added triethylamine and 1-methylpiperazin-2-one (623 mg). The mixture was heated to 50° C. for 16 hours. The reaction mixture was quenched with saturated aqueous sodium bicarbonate mixture and was extracted with ethyl acetate (2×50 mL). The organic phase was concentrated and was purified by silica gel chromatography on a CombiFlash® Teledyne Isco system eluting with 100% ethyl acetate to provide the title compound. LC/MS (ESI) m/z 292 (M+H)⁺.

### Example 58B

### 4-(2-aminoethyl)-1-methylpiperazin-2-one

To a mixture of Example 58A (320 mg) in methanol (5 mL) was added Pd/C (10%, 40 mg). The mixture was degassed, filled with H₂ and stirred at room temperature for 16 hours under an atmosphere of hydrogen gas. The reaction mixture was filtered and concentrated to provide the title compound, which was used in the next step without purification. LC/MS (ESI) m/z 158 (M+H)⁺.

### Example 58C

### (7R,20S)-18-chloro-1-(4-fluorophenyl)-10-{[2-(2-methoxyphenyl)pyrimidin-4-yl]methoxy}-19-methyl-15-[2-(4-methyl-3-oxopiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylic acid

Example 58C was prepared according to the procedure described in Example 23, substituting Example 58B for 2-(4,4-difluoropiperidin-1-yl)ethanamine. ¹H NMR (400 MHz, dimethyl sulfoxide-d₆) δ ppm 8.61 (d, 1H), 7.45 (dtd, 3H), 7.25-7.16 (m, 4H), 7.11 (td, 4H), 7.02 (t, 2H), 6.79 (d, 1H), 6.35 (d, 1H), 5.91 (dd, 1H), 5.21-4.99 (m, 2H), 4.21-3.74 (m, 9H), 3.72 (s, 3H), 3.50-3.06 (m, 8H), 2.85 (s, 3H), 1.73 (s, 3H). MS (ESI) m/z 915 (M+H)⁺.

### Example 59

### ethyl (7R,21S)-19-chloro-1-(4-fluorophenyl)-10-{[2-(2-methoxyphenyl)pyrimidin-4-yl]methoxy}-20-methyl-15-oxo-16-[2-(piperazin-1-yl)ethyl]-7,8,14,15,16,17-hexahydro-18,21-etheno-13,9-(metheno)-6-oxa-2-thia-3,5,16-triazacyclononadeca[1,2,3-cd]indene-7-carboxylate

### Example 59A

### tert-butyl 4-(2-((((9H-fluoren-9-yl)methoxy)carbonyl)(4-bromo-2-chloro-3-methylbenzyl)amino)ethyl)piperazine-1-carboxylate

To a mixture of Example 10A (3.13 g) in dichloromethane (143 mL) with tert-butyl 4-(2-aminoethyl)piperazine-1-carboxylate (3.69 g) was added acetic acid (3.84 mL), sodium cyanoborohydride (1.685 g) and methanol (35.7 mL). The mixture was stirred at ambient temperature for 30 minutes. 9-Fluorenylmethyl chloroformate (4.16 g) was added and stirring was continued for another hour. Triethylamine (15 mL) was added, and the material that formed were redissolved with methanol (50 mL). The resulting mixture was concentrated onto silica gel and purification by silica gel chromatography on a CombiFlash® Teledyne Isco system using a Teledyne Isco RediSep® Rf gold 220 g silica gel column (eluting with 0-70% ethyl acetate/heptane) provided the title compound. LC/MS (APCI) m/z 670.1 (M+H)⁺.

### Example 59B

### benzyl 4-(2-((((9H-fluoren-9-yl)methoxy)carbonyl)(4-bromo-2-chloro-3-methylbenzyl)amino)ethyl)piperazine-1-carboxylate

Example 59A (5.16 g) was dissolved in dichloromethane (38.6 mL), and trifluoroacetic acid (38.6 mL) was added. The mixture was stirred at ambient temperature for 15 minutes and concentrated. Saturated aqueous sodium bicarbonate mixture (40 mL) and 40 mL of tetrahydrofuran were added. While the mixture was stirring, benzyl chloroformate (2.65 mL) was added dropwise. After stirring at ambient temperature for one hour, the mixture was poured into a 500 mL separatory funnel, and was diluted with 200 mL of ethyl acetate and 100 mL of saturated aqueous sodium bicarbonate mixture. The mixture was partitioned, and the aqueous layer was removed. The organic layer was washed with saturated aqueous brine, dried over anhydrous magnesium sulfate, filtered and concentrated onto silica gel. Purification by silica gel flash chromatography on a CombiFlash® Teledyne Isco system using a Teledyne Isco RediSep® Rf gold 220 g silica gel column (eluting with 0-60% ethyl acetate/heptane) provided the title compound. LC/MS (APCI) m/z 704.1 (M+H)⁺.

### Example 59C

### benzyl 4-(2-((4-bromo-2-chloro-3-methylbenzyl)(tert-butoxycarbonyl)amino)ethyl)piperazine-1-carboxylate

Example 59B (4.88 g) was dissolved in tetrahydrofuran (34.7 mL) and methanol (34.7 mL). To the mixture was added 1 molar aqueous lithium hydroxide (69.4 mL) and stirring was continued at ambient temperature for 1 hour. Saturated aqueous sodium bicarbonate mixture (70 mL) and di-tert-butyl dicarbonate (2.42 mL) were added, and the mixture was stirred at ambient temperature for another 90 minutes. The mixture was poured into a 500 mL separatory funnel and was diluted with 200 mL of ethyl acetate and 100 mL of saturated aqueous sodium bicarbonate mixture. The mixture was partitioned, and the aqueous layer was removed. The organic layer was washed with saturated aqueous brine, dried over anhydrous magnesium sulfate, filtered and concentrated onto silica gel. Purification by silica gel chromatography on a CombiFlash® Teledyne Isco system using a Teledyne Isco RediSep® Rf gold 220 g silica gel column (eluting with 10-80% ethyl acetate/heptane) provided the title compound. LC/MS (APCI) m/z 582.1 (M+H)⁺.

### Example 59D

### benzyl 4-(2-((tert-butoxycarbonyl)(2-chloro-3-methyl-4-(4,4,5,5-tetramethyl-1,3,2-dioxaborolan-2-yl)benzyl)amino)ethyl)piperazine-1-carboxylate

The title compound was prepared as described in Example 7H, substituting Example 59C for Example 7G. LC/MS (APCI) m/z 628.3 (M+H)⁺.

### Example 59E

### benzyl 4-(2-((4-((S)-4-(((R)-3-(5-(2-(tert-butoxy)-2-oxoethyl)-2-((2-(2-methoxyphenyl)pyrimidin-4-yl)methoxy)phenyl)-1-ethoxy-1-oxopropan-2-yl)oxy)-6-(4-fluorophenyl)thieno[2,3-d]pyrimidin-5-yl)-2-chloro-3-methylbenzyl)(tert-butoxycarbonyl)amino)ethyl)piperazine-1-carboxylate

The title compound was prepared as described in Example 7N, substituting Example 59D for Example 7H and substituting Example 11C for Example 7M. LC/MS (APCI) m/z 1150.5 (M−Boc+H)⁺.

### Example 59F

### ethyl (7R,21S)-16-(2-{4-[(benzyloxy)carbonyl]piperazin-1-yl}ethyl)-19-chloro-1-(4-fluorophenyl)-10-{[2-(2-methoxyphenyl)pyrimidin-4-yl]methoxy}-20-methyl-15-oxo-7,8,14,15,16,17-hexahydro-18,21-etheno-13,9-(metheno)-6-oxa-2-thia-3,5,16-triazacyclononadeca[1,2,3-cd]indene-7-carboxylate

The title compound was prepared as described in Example 10E, substituting Example 59E for Example 10D. LC/MS (APCI) m/z 1076.3 (M+H)⁺.

### Example 59G

### ethyl (7R,21S)-19-chloro-1-(4-fluorophenyl)-10-{[2-(2-methoxyphenyl)pyrimidin-4-yl]methoxy}-20-methyl-15-oxo-16-[2-(piperazin-1-yl)ethyl]-7,8,14,15,16,17-hexahydro-18,21-etheno-13,9-(metheno)-6-oxa-2-thia-3,5,16-triazacyclononadeca[1,2,3-cd]indene-7-carboxylate

Example 59F (405 mg) was dissolved in methanol (3.8 mL), and palladium hydroxide on carbon (20% weight Degussa® type; 264 mg) was added. The stirring mixture was evacuated and backfilled with nitrogen twice then evacuated and backfilled with hydrogen (using a hydrogen balloon). The mixture was stirred under hydrogen overnight. The mixture was filtered through a 0.45 uM PTFE filter, and the filtrate was concentrated. The residue was purified on Gilson reverse-phase prep HPLC (Zorbax, C-18, 250×21.2 mm column, Mobile phase A: 0.1% trifluoroacetic acid in water; B: 0.1% trifluoroacetic acid in acetonitrile; 10-100% B to A gradient) to provide the title compound. ¹H NMR (400 MHz, dimethyl sulfoxide-d₆) δ ppm 1.13 (t, 3H), 1.87 (s, 3H), 3.06-3.65 (m, 15H), 3.76 (s, 3H), 3.84 (d, 1H), 4.15 (q, 2H), 4.39-4.62 (m, 2H), 4.75-4.88 (m, 2H), 4.93 (d, 1H), 6.55-6.76 (m, 2H), 6.79 (d, 1H), 6.96-7.12 (m, 4H), 7.12-7.22 (m, 3H), 7.21-7.30 (m, 2H), 7.45-7.58 (m, 2H), 8.53 (s, 1H), 8.73 (d, 1H), 9.27 (s, 2H). LC/MS (APCI) m/z 942.2 (M+H)⁺.

### Example 60

### (7S,16R,21S)-19-chloro-1-(4-fluorophenyl)-10-{[2-(2-methoxyphenyl)pyrimidin-4-yl]methoxy}-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid

The title compound was isolated as a minor component during the synthesis of Example 73K. ¹H NMR (500 MHz, dimethylsulfoxide-d₆) δ ppm 9.53 (s, 1H), 8.86 (d, 1H), 8.66 (s, 1H), 7.62 (d, 1H), 7.50 (dd, 1H), 7.44 (ddd, 1H), 7.25-7.15 (m, 4H), 7.13 (d, 1H), 7.02 (td, 1H), 6.97-6.89 (m, 2H), 6.76 (dd, 1H), 6.71 (d, 1H), 5.85 (d, 1H), 5.74 (dd, 1H), 5.25-5.12 (m, 2H), 4.87-4.79 (m, 1H), 4.24 (dd, 1H), 4.14 (dd, 1H), 3.74 (s, 3H), 3.48-3.41 (m, 8H), 3.22-2.97 (m, 2H), 2.97-2.76 (m, 5H), 2.47 (s, 3H). MS (ESI) m/z 903.2 (M+H)⁺.

### Example 61

### (7R,20S)-18-chloro-1-(4-fluorophenyl)-10-{[2-(2-methoxyphenyl)pyrimidin-4-yl]methoxy}-19-methyl-7,8-dihydro-14H,16H-17,20-etheno-13,9-(metheno)-6,15-dioxa-2-thia-3,5-diazacyclooctadeca[1,2,3-cd]indene-7-carboxylic acid

### Example 61A

### 2-(benzyloxy)-5-(hydroxymethyl)benzaldehyde

To a stirred suspension of 2-hydroxy-5-(hydroxymethyl)benzaldehyde (2.48 g) (obtained by following the Stoerner and Behn process, Ber. 1901, 34, 2455-2460) and potassium carbonate (2.5 g) in N,N—N,N-dimethylformamide (10 mL) was added benzyl bromide (2 mL). The mixture was stirred at 40° C. for 14 hours. The mixture was cooled to room temperature, and a mixture of dichloromethane/water (100 mL, 1:1) was added. The layers were separated, and the aqueous layer was extracted with dichloromethane (50 mL×2). The combined organic layers were washed with brine (100 mL×2). The organics were filtered through a Biotage® Isolute Phase Separator column. The organic solvent was removed under reduced pressure. The residue was purified by silica gel chromatography using a Teledyne ISCO CombiFlash® system and ISCO SF40-80 g column, eluting with 0-10% ethyl acetate/heptane, to provide the title compound. MS (ESI) m/z 240.8 (M−H)⁻.

### Example 61B

### 2-(benzyloxy)-5-(((tert-butyldimethylsilyl)oxy)methyl)benzaldehyde

To a mixture of Example 61A (3 g), tert-butyldimethylchlorosilane (2.5 g) and imidazole (1.048 g) was added dichloromethane (20 mL). The mixture was stirred at room temperature for 14 hours. The mixture was filtered, and the material was washed with dichloromethane (2.5 mL×2). The mixture was concentrated under reduced pressure. The reaction mixture was purified by silica gel chromatography using a Teledyne ISCO CombiFlash® system and ISCO SF40-120 g column, eluting with 0-5% ethyl acetate/heptane, to provide the title compound. MS (ESI) m/z 379.2 (M+Na)⁺.

### Example 61C

### ethyl 2-acetoxy-3-(2-(benzyloxy)-5-(((tert-butyldimethylsilyl)oxy)methyl)phenyl)acrylate

To an ice bath cooled mixture of ethyl 2-acetoxy-2-(diethoxyphosphoryl)acetate (2.35 g) in tetrahydrofuran (20 mL) was added lithium chloride (0.73 g) and 1,1,3,3-tetramethylguanidine (2.1 mL). After stirring at 0° C. for 15 minutes, Example 61B (6 g) in tetrahydrofuran (20 mL) was added. The mixture was stirred at room temperature for 2 hours and was quenched by the addition of water (20 mL) and dichloromethane (20 mL). The reaction mixture was filtered through a Biotage® Isolute Phase Separator column and was washed with dichloromethane (5 mL). The solvents were removed under reduced pressure, and the residue was purified by silica gel chromatography using a Teledyne ISCO CombiFlash® system and ISCO SF40-120 g column, eluting with 0-10% ethyl acetate/heptane, to provide the title compound. MS (ESI) m/z 501.9 (M+NH₄)⁺.

### Example 61D

### (R)-ethyl 2-acetoxy-3-(2-(benzyloxy)-5-(((tert-butyldimethylsilyl)oxy)methyl)phenyl)propanoate

In a glovebox, 1,2-bis[(2R,5R)-2,5-diethylphospholano]benzene(1,5-cyclooctadiene)rhodium(I) trifluoromethanesulfonate (0.976 g) was weighed into a vial, and the container was removed. In a 300 mL stainless steel reactor, a mixture of Example 61C (14.06 g) in methanol (150 mL) was prepared and degassed with nitrogen. The reactor was closed, and a mixture of 1,2-Bis[(2R,5R)-2,5-diethylphospholano]benzene(1,5-cyclooctadiene)rhodium(I) trifluoromethanesulfonate in methanol (13 mL) was added via syringe. The reaction mixture was pressurized with hydrogen to 50 psi. After 19 hours, the mixture was filtered and concentrated. The reaction mixture was purified by silica gel chromatography using a Teledyne ISCO CombiFlash® system and ISCO SF65-330 g column, eluting with 0-45% ethyl acetate/heptane, to provide the title compound. MS (ESI) m/z 503.9 (M+NH₄)⁺.

### Example 61E

### (R)-ethyl 2-acetoxy-3-(5-(((tert-butyldimethylsilyl)oxy)methyl)-2-hydroxyphenyl)propanoate

Example 61D (5.7 g) in ethanol (66.2 mL) was added to 5% Pd/C (1.001 g) in a 100 mL Parr stirred reactor. The reactor was purged with nitrogen. The mixture was stirred at 1600 RPM under 50 psi of hydrogen at 25° C. for 6 hours. The reaction mixture was filtered and concentrated under reduced pressure. The residue was dissolved in dichloromethane and loaded to a dry silica gel column, which was dried under reduced pressure. The reaction mixture was purified by silica gel chromatography using a Teledyne ISCO CombiFlash® system and ISCO SF60-330 g column, eluting with 0-30% ethyl acetate/heptane, to provide the title compound. MS (ESI) m/z 413.9 (M+NH₄)⁺.

### Example 61F

### (R)-ethyl 2-acetoxy-3-(5-(((tert-butyldimethylsilyl)oxy)methyl)-2-((2-(2-methoxyphenyl)pyrimidin-4-yl)methoxy)phenyl)propanoate

To a stirred suspension of Example 61E (1.1 g) and triphenylphosphine (1.33 g) in toluene (15 mL) was added (E)-N¹,N¹,N²,N²-tetramethyldiazene-1,2-dicarboxamide (0.87 g). The mixture was stirred at 50° C. for 2 hours. The suspension was filtered and washed with toluene (5 mL×2). The toluene mixture was directly loaded to a RediSep®Rf SF40-80 g silica gel column and purified using a Teledyne ISCO CombiFlash® system, eluting with 10-40% ethyl acetate/heptane, to provide the title compound. MS (ESI) m/z 595.4 (M+H)⁺.

### Example 61G

### (R)-ethyl 3-(5-(((tert-butyldimethylsilyl)oxy)methyl)-2-((2-(2-methoxyphenyl)pyrimidin-4-yl)methoxy)phenyl)-2-hydroxypropanoate

To a stirred mixture of Example 61F (1.5 g) in absolute ethanol (10 mL) was added sodium ethanolate (0.05 mL) (21% w/w in ethanol). The mixture was stirred at room temperature for 1 hour, and acetic acid (0.015 mL) was added. The reaction mixture was diluted with dichloromethane (20 mL) and water (20 mL), and the mixture was filtered through a Biotage® Isolute Phase Separator column and washed with dichloromethane (5 mL×3). The solvents were removed under reduced pressure, and the title compound was used directly in next step without further purification. MS (ESI) m/z 553.3 (M+H)⁺.

### Example 61H

### (R)-ethyl 2-((5-bromo-6-(4-fluorophenyl)thieno[2,3-d]pyrimidin-4-yl)oxy)-3-(5-(((tert-butyldimethylsilyl)oxy)methyl)-2-((2-(2-methoxyphenyl)pyrimidin-4-yl)methoxy)phenyl)propanoate

To a stirred suspension of Example 61G (1.4 g) and cesium carbonate (2.5 g) in tert-butanol (10 mL) was added Example 1D (1.0 g). The mixture was stirred at 65° C. for 3 hours. The reaction mixture was cooled to room temperature, and diethyl ether (100 mL) was added. The mixture was filtered, and the material was washed with diethyl ether (10 mL×3). The combined diethyl ether filtrate was concentrated under reduced pressure. The residue was dissolved in dichloromethane (5 mL), loaded onto a dry silica gel column (RedSep Gold, SF40-80 g), and dried under reduced pressure. The reaction mixture was purified by silica gel chromatography using a Teledyne ISCO CombiFlash® system, eluting with 1-10% ethyl acetate/heptane, to provide the title compound. MS (ESI) m/z 859.2 (M+H)⁺.

### Example 61I

### (2R)-ethyl 3-(5-(((tert-butyldimethylsilyl)oxy)methyl)-2-((2-(2-methoxyphenyl)pyrimidin-4-yl)methoxy)phenyl)-2-((5-((1S)-4-(((tert-butyldimethylsilyl)oxy)methyl)-3-chloro-2-methylphenyl)-6-(4-fluorophenyl)thieno[2,3-d]pyrimidin-4-yl)oxy)propanoate

To a stirred suspension of Example 61H (0.2 g), Example 20G (0.15 g), bis(di-tert-butyl(4-dimethylaminophenyl)phosphine)dichloropalladium (II) (0.02 g) and potassium phosphate (0.15 g) in tetrahydrofuran (1 mL) and water (0.3 mL) was degassed by three cycles of reduced pressure/nitrogen backfill. The suspension was stirred at room temperature for 20 hours. Dichloromethane (20 mL) and water (20 mL) were added, and the mixture was filtered through a Biotage® Isolute Phase Separator column. The solvents were removed by reduced pressure, and the reaction mixture was purified by silica gel chromatography using a Teledyne ISCO CombiFlash® system and RediSep® SF15-40 g Gold column, eluting with 10-50% ethyl acetate/heptane, to provide the title compound. MS (ESI) m/z 1049.3 (M+H)⁺.

### Example 61J

### (R)-ethyl 2-((5-((1S)-3-chloro-4-(hydroxymethyl)-2-methylphenyl)-6-(4-fluorophenyl)thieno[2,3-d]pyrimidin-4-yl)oxy)-3-(5-(hydroxymethyl)-2-((2-(2-methoxyphenyl)pyrimidin-4-yl)methoxy)phenyl)propanoate

To a stirred mixture of Example 61I (0.174 g) in tetrahydrofuran (1 mL) was added tetra-N-butylammonium fluoride (0.5 mL, 1M in tetrahydrofuran). The mixture was stirred at room temperature for 1 hour. Ethyl acetate (30 mL) was added, and the mixture was washed with brine. The aqueous layer was extracted with ethyl acetate (10 mL). The combined organic phase was filtered through a Biotage® Isolute Phase Separator column, and the solvents were removed under reduced pressure. The residue was purified by silica gel chromatography using a Teledyne ISCO CombiFlash® system and RediSep® Rf SF40-120 g Gold column, eluting with 20-50% ethyl acetate/heptane, to provide the title compound. MS (ESI) m/z 821.3 (M+H)⁺.

### Example 61K

### ethyl (7R,20S)-18-chloro-1-(4-fluorophenyl)-10-{[2-(2-methoxyphenyl)pyrimidin-4-yl]methoxy}-19-methyl-7,8-dihydro-14H,16H-17,20-etheno-13,9-(metheno)-6,15-dioxa-2-thia-3,5-diazacyclooctadeca[1,2,3-cd]indene-7-carboxylate

A mixture of Example 61J (0.067 g) and 2-(tributylphosphoranylidene)acetonitrile (0.1 g) was dissolved in toluene (5 mL) and stirred at 75° C. for 3 hours. The reaction mixture was directly loaded onto a RediSep® SF15-24 g Gold column and purified using a Teledyne ISCO CombiFlash® system, eluting with 10-70% ethyl acetate/heptane, to provide the title compound. MS (ESI) m/z 803.3 (M+H)⁺.

### Example 61L

### (7R,20S)-18-chloro-1-(4-fluorophenyl)-10-{[2-(2-methoxyphenyl)pyrimidin-4-yl]methoxy}-19-methyl-7,8-dihydro-14H,16H-17,20-etheno-13,9-(metheno)-6,15-dioxa-2-thia-3,5-diazacyclooctadeca[1,2,3-cd]indene-7-carboxylic acid

A mixture of Example 61K (13.5 mg) and lithium hydroxide hydrate mixture (5 mg in 1 mL water) in methanol (10 mL) was stirred at room temperature overnight. After removal of the solvents under reduced pressure, acetonitrile (1 mL) with trifluoroacetic acid (10 μL) was added to the residue. The reaction mixture was purified by reverse phase HPLC using a Gilson system (Luna column, 250×30 mm, flow rate 50 mL/min) using a gradient of 50% to 100% acetonitrile water with 0.1% trifluoroacetic acid over 30 minutes. The product containing fractions were lyophilized to provide the title compound. ¹H NMR (400 MHz, dimethyl sulfoxide-d₆) δ ppm 8.59 (m, 2H), 7.43 (m, 4H), 7.20 (m, 4H), 7.11 (m, 3H), 7.00 (m, 2H), 6.73 (d, 1H), 6.41 (d, 1H), 5.85 (dd, 1H), 5.08 (q, 2H), 4.79 (d, 1H), 4.52 (m, 3H), 3.72 (s, 3H), 3.11 (m, 2H), 1.66 (s, 3H). MS (ESI) m/z 775.2 (M+H)⁺.

### Example 62

### (7R,20S)-18-chloro-1-(4-fluorophenyl)-10-{[2-(2-methoxyphenyl)pyrimidin-4-yl]methoxy}-19-methyl-15-[2-(piperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylic acid

To a mixture of Example 44 (26 mg) in tetrahydrofuran (230 μL) and methanol (230 μL) was added a mixture of lithium hydroxide (7.4 mg) in water (230 μL), and the reaction mixture was allowed to stir overnight. The reaction mixture was quenched with trifluoroacetic acid (40 μL, 25 equiv.) and was diluted with dimethyl sulfoxide (600 μL). The mixture was purified by reverse-phase HPLC on a Gilson PLC 2020 using a Luna column (250×50 mm, 10 mm) (5-70% over 30 minutes with acetonitrile in water containing 0.1% trifluoroacetic acid) to provide the title compound after lyophilization. ¹H NMR (400 MHz, dimethyl sulfoxide-d₆) δ ppm 9.02 (br s, 1H), 8.70-8.61 (m, 2H), 7.57-7.40 (m, 3H), 7.33-7.09 (m, 9H), 7.05 (t, 1H), 6.85 (d, 1H), 6.45 (d, 1H), 5.96 (dd, 1H), 5.14 (dd, 2H), 4.30 (dd, 2H), 4.13 (s, 2H), 3.75 (s, 3H), 3.57-3.40 (m, 2H), 3.31-2.97 (m, 12H), 1.75 (s, 3H). MS (ESI) m/z 886.4 (M+H)⁺.

### Example 63

### (7R,16R,21R)-19-chloro-1-(4-fluorophenyl)-10-{[2-(2-methoxyphenyl)pyrimidin-4-yl]methoxy}-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid

The title compound was isolated during the preparation of Example 68G. ¹H NMR (501 MHz, dimethyl sulfoxide-d₆) δ ppm 9.55 (br s, 1H), 8.85 (d, 1H), 8.61 (s, 1H), 7.65 (d, 1H), 7.50 (dd, 1H), 7.49-7.40 (m, 1H), 7.33-7.27 (m, 2H), 7.24-7.17 (m, 2H), 7.13 (dd, 1H), 7.07-7.00 (m, 2H), 6.84 (d, 1H), 6.75 (dd, 1H), 6.63 (d, 1H), 6.04 (d, 1H), 5.75 (dd, 1H), 5.25-5.08 (m, 3H), 4.38 (d, 1H), 4.07 (dd, 1H), 3.74 (s, 3H), 3.32-3.17 (m, 3H), 3.08 (s, 2H), 2.90 (td, 2H), 2.79 (s, 3H), 2.55 (m, 2H), 2.46 (s, 3H).

### Example 64

### (7R,16S,21S)-19-chloro-1-(4-fluorophenyl)-20-methyl-16-[(4-methylpiperazin-1-yl)methyl]-10-{[2-(3,3,3-trifluoropropoxy)pyrimidin-4-yl]methoxy}-7,8,15,16-tetrahydro-18,21-etheno-13,9-(metheno)-6,14,17-trioxa-2-thia-3,5-diazacyclononadeca[1,2,3-cd]indene-7-carboxylic acid

### Example 64A

### (4-bromo-2-chlorophenoxy)triisopropylsilane

To a mixture of 4-bromo-2-chlorophenol (570 g) in dichloromethane (4.5 L) was added triisopropylchlorosilane (582 mL) and imidazole (187 g), and the mixture was stirred for 8 hours at 25° C. The reaction mixture was poured into water, and extracted with dichloromethane (3×2000 mL). The organic layers were combined, washed with brine (1×2000 mL), dried over anhydrous sodium sulfate, filtered and concentrated under reduced pressure to give a residue. The residue was purified by column chromatography on silica gel, eluting with petroleum ether to obtain the title compound. ¹H NMR (400 MHz, chloroform-d) δ ppm 1.12 (d, 18H), 1.27-1.35 (m, 3H), 6.78 (d, 1H), 7.21 (dd, 1H), 7.49 (d, 1H).

### Example 64B

### (4-bromo-2-chloro-3-methylphenoxy)triisopropylsilane

A 5 L, 3-neck round-bottom flask, fitted with overhead stirring, nitrogen inlet and outlet, three addition funnels, a thermocouple and a Claisen adaptor was twice dried with a torch and heat gun and cooled under nitrogen. The reaction flask was charged with N,N-diisopropylamine (69.2 mL) and tetrahydrofuran (2110 mL). The mixture was cooled to −78° C. under nitrogen. n-Butyllithium (177 mL, 2.5 M in hexane) was added slowly via addition funnel, and a slight rise in temperature was observed. The mixture was stirred at −78° C. for 45 minutes, at which time Example 64A (153.5 g) was added over 30 minutes as a tetrahydrofuran (200 mL) mixture. The reaction mixture was stirred for about 6.5 hours at −76° C. Iodomethane (31.7 mL) was added dropwise via addition funnel maintaining the temperature below −62° C. The reaction mixture was allowed to warm slowly overnight to room temperature. The volatiles were removed by rotary evaporation. Ethyl acetate (1.5 L) and water (1.5 L) were added to the residue, and the layers were separated. The organics were washed with brine. The combined aqueous layer was extracted once with ethyl acetate (500 mL). The combined organics were dried (MgSO₄), filtered and concentrated by rotary evaporation. The residue was purified by flash silica gel column chromatography (1500 g SiO₂, heptanes) to provide the title compound.

### Example 64C

### 4-bromo-2-chloro-3-methylphenol

To a mixture of Example 64B (500 g) in tetrahydrofuran (5 L) was added tetra-N-butylammonium fluoride (381 g). The reaction mixture was stirred at 25° C. for 3 hours. The reaction mixture was diluted with water (3 L), and extracted with tert-butyl methyl ether (3×2 L). The combined organic layers were dried over anhydrous sodium sulfate, filtered and concentrated under reduced pressure. The residue was diluted with 10% (w/w) aqueous sodium hydroxide (8 L) and washed with a mixture of petroleum ether/tert-butyl methyl ether (v/v=10/1, 3×3 L). The organic layer was discarded. The aqueous layer was adjusted to pH=3 with 3 N aqueous HCl mixture and was extracted with a mixture of petroleum ether/tert-butyl methyl ether (v/v=10/1, 3×4 L). The combined organic layers were dried over anhydrous sodium sulfate, filtered and concentrated under reduced pressure to give a residue. The residue was triturated with petroleum ether (1.5 L), and the material was dried under high vacuum to provide the title compound. ¹H NMR (400 MHz, chloroform-d) δ ppm 2.51 (s, 3H) 5.60 (s, 1H) 6.80 (d, 1H) 7.37 (d, 1H).

### Example 64D

### (R)-(2,2-dimethyl-1,3-dioxolan-4-yl)methyl benzoate

(S)-(+)-2,2-Dimethyl-1,3-dioxolane-4-methanol (3.0 g) was stirred in pyridine (92 mL). Benzoic anhydride (10.3 g) and 4-dimethylaminopyridine (0.92 g) were added. The mixture was stirred at ambient temperature under nitrogen for 90 minutes. The mixture was concentrated to remove most of the pyridine and was dissolved in diethyl ether (˜80 mL). A 5% aqueous ammonium hydroxide mixture (100 mL) was added, and the biphasic mixture was vigorously stirred at ambient temperature for 10 minutes. The mixture was poured into a separatory funnel, and was diluted with 5% aqueous ammonium hydroxide mixture (200 mL) and diethyl ether (200 mL). The mixture was partitioned between the two phases. The aqueous layer was removed. The organic layer was washed with 1 molar aqueous hydrochloric acid mixture and saturated aqueous brine, dried over anhydrous magnesium sulfate, filtered and concentrated onto silica gel. Purification by silica gel flash chromatography on a CombiFlash® Teledyne Isco system using a Teledyne Isco RediSep® Rf gold 220 g silica gel column (eluting with 0-40% ethyl acetate/heptane) provided the title compound. ¹H NMR (400 MHz, dimethyl sulfoxide-d₆) δ ppm 1.29 (d, 6H), 3.73-3.87 (m, 1H), 4.01-4.11 (m, 1H), 4.20-4.32 (m, 1H), 4.31-4.45 (m, 2H), 7.45-7.59 (m, 2H), 7.60-7.70 (m, 1H), 7.92-8.03 (m, 2H).

### Example 64E

### (R)-2,3-dihydroxypropyl benzoate

Antimony trichloride (1.45 g) and water (0.76 mL) were added to a stirring mixture of Example 64D (5.0 g) in acetonitrile (212 mL). The reaction mixture was stirred at ambient temperature for 30 minutes and was concentrated onto silica gel. Purification by silica gel chromatography on a CombiFlash® Teledyne Isco system using a Teledyne Isco RediSep® Rf gold 220 g silica gel column (eluting with 0-60% 2:1 ethyl acetate:ethanol/heptane) provided the title compound. LC/MS (APCI) m/z 197.4 (M+H)⁺.

### Example 64F

### (R)-3-(bis(4-methoxyphenyl)(phenyl)methoxy)-2-hydroxypropyl benzoate

Example 64E (4.14 g) was dissolved in pyridine (129 mL), and N,N-diisopropylethylamine (8.84 mL) was added followed by 4-dimethylaminopyridine (1.3 g). To this stirring mixture was slowly added 4,4′-dimethoxytrityl chloride (10.7 g) as a pyridine (64.5 mL) mixture over 40 minutes. Stirring continued at ambient temperature for 12 hours. The mixture was concentrated under reduced pressure, and the residue was dissolved in ethyl acetate. The mixture was washed with water and brine, dried over anhydrous magnesium sulfate, filtered and concentrated onto silica gel. Purification by silica gel chromatography on a CombiFlash® Teledyne Isco system using a Teledyne Isco RediSep® Rf gold 220 g silica gel column (eluting 0-40% ethyl acetate/heptane) provided the title compound. Analytical SFC was performed on an Aurora A5 SFC Fusion and Agilent 1100 system running under Agilent Chemstation software control. The SFC system included a 10-way column switcher, CO₂ pump, modifier pump, oven, and backpressure regulator. The mobile phase comprised of supercritical CO₂ supplied by a beverage-grade CO₂ cylinder with a modifier mixture of methanol at a flow rate of 3 mL/minute. Oven temperature was at 35° C. and the outlet pressure at 150 bar. The mobile phase gradient started with 5% modifier and held it for 0.1 minutes at a flow rate of 1 mL/minute, then the flow rate was ramped up to 3 mL/minute and held for 0.4 minutes. The modifier was ramped from 5% to 50% over the next 8 minutes at 3 mL/minute then held for 1 minute at 50% modifier (3 mL/minute). The gradient was ramped down from 50% to 5% modifier over 0.5 minute (3 mL/minute). The instrument was fitted with a ChiralCel OJ-H column with dimensions of 4.6 mm i.d.×150 mm length with 5 μm particles. Minor enantiomer (S) eluted after 5.1 minutes and major enantiomer (R) eluted after 6.1 minutes. Using this assay the title compound enantiopurity was determined to be 97% ee (enantiomeric excess). ¹H NMR (400 MHz, dimethyl sulfoxide-d₆) δ ppm 3.03 (d, 2H), 3.67 (d, 6H), 3.90-4.00 (m, 1H), 4.23-4.39 (m, 2H), 5.20 (d, 1H), 6.74-6.84 (m, 4H), 7.14-7.26 (m, 7H), 7.33-7.40 (m, 2H), 7.44-7.51 (m, 2H), 7.59-7.66 (m, 1H), 7.79-7.86 (m, 2H).

### Example 64G

### (S)-3-(bis(4-methoxyphenyl)(phenyl)methoxy)-2-(4-bromo-2-chloro-3-methylphenoxy)propyl benzoate

A 500 mL round bottom flask, equipped with stir bar and septum, was charged with Example 64F (5.62 g), Example 64C (3.25 g), di-tert-butyl azodicarboxylate (3.89 g) and triphenylphosphine (4.43 g). The flask was evacuated and backfilled with nitrogen twice. Tetrahydrofuran (113 mL) was introduced via syringe, and the flask was evacuated and backfilled with nitrogen twice again and was stirred at 45° C. for 2 hours. After cooling to ambient temperature, the mixture was concentrated onto silica gel and purified by silica gel chromatography on a CombiFlash® Teledyne Isco system using a Teledyne Isco RediSep® Rf gold 330 g silica gel column (eluting 0-30% ethyl acetate/heptane) to provide the title compound. Analytical SFC was performed on an Aurora A5 SFC Fusion and Agilent 1100 system running under Agilent Chemstation software control. The SFC system included a 10-way column switcher, CO₂ pump, modifier pump, oven, and backpressure regulator. The mobile phase comprised of supercritical CO₂ supplied by a beverage-grade CO₂ cylinder with a modifier mixture of methanol at a flow rate of 3 mL/minute. Oven temperature was at 35° C. and the outlet pressure at 150 bar. The mobile phase gradient started with 40% modifier, held for 0.1 minutes at a flow rate of 1 mL/minute, then the flow rate was ramped up to 3 mL/minute and held for 0.4 minutes. The modifier was ramped from 40% to 50% over the next 8 minutes at 3 mL/minute then held for 1 minute at 50% modifier (3 mL/minute). The gradient was ramped down from 50% to 5% modifier over 0.5 minute (3 mL/minute). The instrument was fitted with a ChiralCel OJ-H column with dimensions of 4.6 mm i.d.×150 mm length with 5 μm particles. Minor enantiomer (R) eluted after 3.8 minutes and major enantiomer (S) eluted after 5.7 minutes. Using this assay the title compound enantiopurity was determined to be 97% ee (enantiomeric excess). ¹H NMR (400 MHz, dimethyl sulfoxide-d₆) δ ppm 2.41 (s, 3H), 3.32 (s, 2H), 4.57 (d, 2H), 4.99 (p, 1H), 6.75-6.86 (m, 4H), 7.11 (d, 1H), 7.15-7.28 (m, 7H), 7.31-7.38 (m, 2H), 7.42-7.52 (m, 3H), 7.58-7.68 (m, 1H), 7.70-7.78 (m, 2H).

### Example 64H

### (R)-3-(bis(4-methoxyphenyl)(phenyl)methoxy)-2-(4-bromo-2-chloro-3-methylphenoxy)propan-1-ol

To a tetrahydrofuran (96 mL) mixture of Example 64G (6.75 g) was added lithium hydroxide (96 mL, 1 M) followed by 20 mL of methanol, and the mixture was allowed to stir at ambient temperature for 1 hour. The mixture was diluted with ethyl acetate and washed with saturated aqueous sodium bicarbonate mixture (once), brine, dried over anhydrous magnesium sulfate, filtered and concentrated onto silica gel. Purification by silica gel chromatography on a CombiFlash® Teledyne Isco system using a Teledyne Isco RediSep® Rf gold 120 g silica gel column (eluting with 0-50% ethyl acetate/heptane) provided the title compound. ¹H NMR (501 MHz, dimethyl sulfoxide-d₆) δ ppm 2.45 (s, 3H), 3.21 (d, 2H), 3.51-3.67 (m, 2H), 3.70 (d, 6H), 4.57 (p, 1H), 4.88 (t, 1H), 6.78-6.85 (m, 4H), 7.05 (d, 1H), 7.14-7.20 (m, 5H), 7.21-7.28 (m, 2H), 7.28-7.33 (m, 2H), 7.49 (d, 1H).

### Example 64I

### (S)-3-(bis(4-methoxyphenyl)(phenyl)methoxy)-2-(4-bromo-2-chloro-3-methylphenoxy)propyl 4-methylbenzenesulfonate

A mixture of Example 64H (3.18 g) and triethylamine (1.11 mL) in dichloromethane (53 mL), was cooled with an ice-water bath, and para-toluenesulfonyl chloride (1.2 g) was added in one portion. The cooling bath was removed, and the mixture was stirred at ambient temperature for 12 hours. The reaction mixture was concentrated onto silica gel and purification by silica gel chromatography on a CombiFlash® Teledyne Isco system using a Teledyne Isco RediSep® Rf gold 120 g silica gel column (eluting with 0-40% ethyl acetate/heptane) provided the title compound. ¹H NMR (400 MHz, dimethyl sulfoxide-d₆) δ ppm 2.33 (s, 3H), 2.41 (s, 3H), 3.16 (d, 2H), 3.69 (d, 6H), 4.19-4.31 (m, 2H), 4.75 (p, 1H), 6.74-6.86 (m, 5H), 7.06-7.12 (m, 4H), 7.13-7.20 (m, 1H), 7.20-7.25 (m, 4H), 7.31-7.37 (m, 2H), 7.39 (d, 1H), 7.61-7.70 (m, 2H)

### Example 64J

### (R)-1-(3-(bis(4-methoxyphenyl)(phenyl)methoxy)-2-(4-bromo-2-chloro-3-methylphenoxy)propyl)-4-methylpiperazine

To a mixture of Example 64I (3.7 g) and triethylamine (2.057 mL) in N,N-dimethylformamide (50 mL) was added 1-methylpiperazine (2.7 mL) in one portion, and the reaction mixture was stirred at 80° C. for 12 hours. After cooling to ambient temperature, the reaction mixture was poured into a separatory funnel and was diluted with ethyl acetate. The mixture was washed with water and brine, dried over anhydrous magnesium sulfate, filtered and concentrated onto silica gel. Purification by flash chromatography on a CombiFlash® Teledyne Isco system using a Teledyne Isco RediSep® Rf gold 120 g silica gel column (eluting with 10-100% 2:1 ethyl acetate:ethanol/heptane) provided the title compound. ¹H NMR (500 MHz, dimethyl sulfoxide-d₆) δ ppm 2.07 (s, 3H), 2.10-2.25 (m, 4H), 2.30-2.43 (m, 4H), 2.45 (s, 3H), 2.58 (dd, 1H), 2.66 (dd, 1H), 3.16 (dd, 1H), 3.25 (dd, 1H), 3.71 (d, 6H), 4.60-4.75 (m, 1H), 6.77-6.85 (m, 4H), 7.02 (d, 1H), 7.15-7.21 (m, 5H), 7.21-7.27 (m, 2H), 7.30-7.35 (m, 2H), 7.45 (d, 1H).

### Example 64K

### (R)-1-(3-(bis(4-methoxyphenyl)(phenyl)methoxy)-2-(2-chloro-3-methyl-4-(4,4,5,5-tetramethyl-1,3,2-dioxaborolan-2-yl)phenoxy)propyl)-4-methylpiperazine

The title compound was prepared as described in Example 7H, substituting Example 64J for Example 7G. ¹H NMR (400 MHz, dimethyl sulfoxide-d₆) δ ppm 1.26 (s, 12H), 2.05 (s, 3H), 2.08-2.22 (m, 4H), 2.27-2.44 (m, 4H), 2.51 (s, 3H), 2.57 (dd, 1H), 2.66 (dd, 1H), 3.13 (dd, 1H), 3.22 (dd, 1H), 3.68 (d, 6H), 4.69 (p, 1H), 6.71-6.82 (m, 4H), 6.97 (d, 1H), 7.11-7.25 (m, 7H), 7.27-7.32 (m, 2H), 7.47 (d, 1H).

### Example 64L

### (R)-ethyl 2-((5-((1S)-4-(((R)-1-(bis(4-methoxyphenyl)(phenyl)methoxy)-3-(4-methylpiperazin-1-yl)propan-2-yl)oxy)-3-chloro-2-methylphenyl)-6-(4-fluorophenyl)thieno[2,3-d]pyrimidin-4-yl)oxy)-3-(5-((tert-butyldimethylsilyl)oxy)-2-((2-(3,3,3-trifluoropropoxy)pyrimidin-4-yl)methoxy)phenyl)propanoate

The title compound was prepared as described in Example 7N, substituting Example 16G for Example 7M, and also substituting Example 64K for Example 7H. From this reaction mixture was obtained an inseparable 3:1 mixture of atropisomers with the major isomer being the title compound. LC/MS (APCI) m/z 1070.4 (M-dimethoxytrityl+H)⁺.

### Example 64M

### (R)-ethyl 3-(5-((tert-butyldimethylsilyl)oxy)-2-((2-(3,3,3-trifluoropropoxy)pyrimidin-4-yl)methoxy)phenyl)-2-((5-((1S)-3-chloro-4-(((R)-1-hydroxy-3-(4-methylpiperazin-1-yl)propan-2-yl)oxy)-2-methylphenyl)-6-(4-fluorophenyl)thieno[2,3-d]pyrimidin-4-yl)oxy)propanoate

To a stirring mixture of Example 64L (115 mg) in dichloromethane (0.8 mL) and methanol (0.8 mL) was added 0.8 mL of formic acid, and the mixture was stirred at ambient temperature for 30 minutes. The mixture was carefully poured into 10 mL of saturated aqueous sodium bicarbonate. The resulting mixture was poured into a separatory funnel, diluted with ethyl acetate and partitioned between the two phases. The aqueous layer was removed, and the organic layer was washed with saturated aqueous brine, dried over magnesium sulfate, filtered and concentrated onto silica gel. Purification by silica gel chromatography on a CombiFlash® Teledyne Isco system using a Teledyne Isco RediSep® Rf gold 12 g silica gel column (eluting with 0-20% 2:1 ethyl acetate:water/ethyl acetate) provided the title compound. LC/MS (APCI) m/z 1069.3 (M+H)⁺.

### Example 64N

### ethyl (7R,16S,21S)-19-chloro-1-(4-fluorophenyl)-20-methyl-16-[(4-methylpiperazin-1-yl)methyl]-10-{[2-(3,3,3-trifluoropropoxy)pyrimidin-4-yl]methoxy}-7,8,15,16-tetrahydro-18,21-etheno-13,9-(metheno)-6,14,17-trioxa-2-thia-3,5-diazacyclononadeca[1,2,3-cd]indene-7-carboxylate

A stirring mixture of Example 64M (20 mg) and triethylamine (8 μL) in dichloromethane (200 μL) was cooled in an ice-water bath and para-toluenesulfonyl chloride (7 mg) was added in one portion. The cooling bath was removed, and the mixture was stirred at ambient temperature for four hours. The reaction mixture was concentrated to remove most of the dichloromethane and was treated with tetra-N-butylammonium fluoride (1 molar in tetrahydrofuran, 300 μL). The mixture stirred at ambient temperature for 3 hours. The mixture was concentrated and was purified by silica gel preparative thin-layer chromatography (0.5 mm thick, 20×20 cm, eluting with 15% 2:1 methanol:water in ethyl acetate) to provide the title compound. LC/MS (APCI) m/z 937.1 (M+H)⁺.

### Example 64O

### (7R,16S,21S)-19-chloro-1-(4-fluorophenyl)-20-methyl-16-[(4-methylpiperazin-1-yl)methyl]-10-{[2-(3,3,3-trifluoropropoxy)pyrimidin-4-yl]methoxy}-7,8,15,16-tetrahydro-18,21-etheno-13,9-(metheno)-6,14,17-trioxa-2-thia-3,5-diazacyclononadeca[1,2,3-cd]indene-7-carboxylic acid

The title compound was prepared as described in Example 10F, substituting Example 64N for Example 10E. ¹H NMR (400 MHz, dimethyl sulfoxide-d₆) δ ppm 2.18 (s, 3H), 2.54 (s, 3H), 2.71-2.97 (m, 6H), 2.98-3.55 (m, 8H), 3.80 (dd, 1H), 3.97 (t, 1H), 4.40 (d, 1H), 4.53 (t, 2H), 4.92-5.26 (m, 2H), 5.79 (d, 1H), 6.28 (dd, 1H), 6.70 (dd, 1H), 6.83 (d, 1H), 6.93 (d, 1H), 7.13-7.29 (m, 6H), 8.62 (d, 1H), 8.74 (s, 1H). LC/MS (APCI) m/z 909.1 (M+H)⁺.

### Example 65

### (7R,20S)-18-chloro-1-(4-fluorophenyl)-10-({2-[2-(2-methoxyethoxy)phenyl]pyrimidin-4-yl}methoxy)-19-methyl-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylic acid

### Example 65A

### 2-(2-methoxyethoxy)benzonitrile

To a mixture of 2-hydroxybenzonitrile (82 g) in N,N-dimethylformamide (2.5 L) was added 1-bromo-2-methoxyethane (96 g) and cesium fluoride (299 g). The mixture was stirred at 25° C. for 12 hours. The mixture was filtered, and the solvent was evaporated under reduced pressure to provide the title compound, which was used in the subsequent reaction without further purification. ¹H NMR (400 MHz, chloroform-d) δ ppm 7.63-7.38 (m, 2H), 7.05-6.92 (m, 2H), 4.22-4.19 (m, 2H), 3.811-3.76 (m, 2H), 3.49-3.38 (m, 3H).

### Example 65B

### 2-(2-methoxyethoxy)benzimidamide

To a mixture of Example 65A (50 g) in methanol (500 mL) was bubbled in HCl gas for 0.5 hours at −50° C. The reaction mixture was stirred at 25° C. for 24 hours. The reaction mixture was diluted with ethyl acetate and was filtered. The solvent was evaporated under reduced pressure to give an intermediate product, which was dissolved in methanol (400 mL) and bubbled with ammonia gas for 0.5 hour at −50° C. The reaction mixture was stirred at 25° C. for 24 hours. The mixture was filtered, and the solvent was evaporated under reduced pressure to provide the title compound. MS (ESI) m/z 210 (M+H)⁺.

### Example 65C

### (4-(dimethoxymethyl)-2-(2-(2-methoxyethoxy)phenyl)pyrimidine

To a mixture of Example 65B (40 g) in methanol (250 mL) was added (E)-4-(dimethylamino)-1,1-dimethoxybut-3-en-2-one (38.5 g) and sodium methoxide (12.02 g), and the mixture was stirred at 75° C. for 12 hours. The mixture was cooled to 25° C. and was concentrated under reduced pressure. The residue was diluted with water (500 mL) and extracted with dichloromethane (3×400 mL). The combined organic layers were dried over sodium sulfate, filtered and concentrated under reduced pressure to provide the title compound, which was used in the subsequent step without further purification. ¹H NMR (400 MHz, chloroform-d) δ ppm 8.83 (d, 1H), 7.68 (d, 1H), 7.42 (d, 1H), 7.35 (t, 1H), 7.07-6.97 (m, 2H), 5.30 (s, 1H), 4.22-4.10 (m, 2H), 3.66 (t, 2H), 3.42 (s, 6H), and 3.29 (s, 3H).

### Example 65D

### (2-(2-(2-methoxyethoxy)phenyl)pyrimidin-4-yl)methanol

To a mixture of Example 65C (25 g) in HCl/1,4-dioxane (4 M, 140 mL) was added water (210 mL) at 25° C. The mixture was heated to 50° C. for 16 hours. The reaction mixture was cooled to 0° C., and solid sodium hydroxide (33.6 g) was added portionwise at 0° C. The pH was adjusted to 8 using 10% potassium carbonate, and sodium borohydride (6.22 g) was added. The mixture was stirred for 30 minutes at 0° C. The mixture was diluted with 200 mL water and was extracted with ethyl acetate (3×300 mL). The combined organic phases were dried over sodium sulfate, filtered, and concentrated under reduced pressure. The residue was purified by column chromatography on silica gel, eluting with 1:5 petroleum ether:ethyl acetate to provide the title compound. ¹H NMR (400 MHz, chloroform-d) δ ppm 8.85-8.62 (m, 1H), 7.81 (dd, 1H), 7.43-7.34 (m, 1H), 7.12 (d, 1H), 7.09-6.99 (m, 2H), 4.74 (br. s., 2H), 4.25-4.13 (m, 3H), 3.74-3.65 (m, 2H), 3.35 (s, 3H).

### Example 65E

### 4-(chloromethyl)-2-(2-(2-methoxyethoxy)phenyl)pyrimidine

To a mixture of Example 65D (300 mg) in anhydrous dichloromethane (20 mL) was added triphenylphosphine (393 mg) at 0° C. The mixture was stirred at 0° C. for 45 minutes, and N-chlorosuccinimide (169 mg) was added. The reaction mixture was warmed to room temperature for 3 hours, and was directly loaded onto a silica gel column that was eluted with 20-60% ethyl acetate in heptane to provide the title compound. MS (ESI) m/z 278 (M+H)⁺.

### Example 65F

### (R)-ethyl 2-acetoxy-3-(5-bromo-2-((4-methoxybenzyl)oxy)phenyl)propanoate

A mixture of 4-methoxybenzyl alcohol (6.51 g), triphenylphosphine (12.36 g), Example 1K (12.0 g) and N,N,N,N′-tetramethylazodicarboxamide (8.11 g) were dissolved in anhydrous toluene (200 mL) at 0° C. The mixture was stirred at 0° C. for 2 hours and was allowed to warm to room temperature overnight. The reaction mixture was directly purified by silica gel chromatography (330 g RediSep® Gold column, 10-40% ethyl acetate in hexane) to provide the title compound. MS (ESI) m/z 470 (M+NH₄)⁺.

### Example 65G

### (R,E)-ethyl 2-acetoxy-3-(2-((4-methoxybenzyl)oxy)-5-(pent-1-en-1-yl)phenyl)propanoate

A mixture of Example 65F (10.12 g), (E)-pent-1-en-1-ylboronic acid (5.11 g), 2-dicyclohexylphosphino-2′,6′-dimethoxybiphenyl (1.289 g), palladium(II) acetate (0.503 g) and cesium fluoride (10.22 g) in a 500 mL round-bottom flask was purged with nitrogen. Anhydrous 1,4-dioxane (200 mL) was added under nitrogen. The mixture was purged with nitrogen again and was stirred at room temperature for 4 hours. The mixture was partitioned between ethyl acetate (400 mL) and brine (500 mL). The organic phase was washed with brine and was concentrated. The residue was purified by silica gel chromatography (5-30% ethyl acetate in heptane) to provide the title compound. MS (ESI) m/z 458 (M+NH₄)⁺.

### Example 65H

### (R)-ethyl 2-acetoxy-3-(5-formyl-2-((4-methoxybenzyl)oxy)phenyl)propanoate

To Example 65G (9.68 g) and iodobenzene diacetate (15.78 g) in a mixture of tetrahydrofuran (170 mL) and water (8.5 mL) was added 2,6-dimethylpiperidine (6.55 mL) and osmium tetroxide (0.1 M mixture in water, 4.26 mL). The reaction mixture was stirred at room temperature for 4 hours. The reaction mixture was partitioned between ethyl acetate and brine. The organic phase was washed with brine and was concentrated. The residue was purified by silica gel chromatography (5-40% ethyl acetate in heptane) to provide the title compound. MS (ESI) m/z 418 (M+NH₄)⁺.

### Example 65I

### (R)-ethyl 3-(5-formyl-2-((4-methoxybenzyl)oxy)phenyl)-2-hydroxypropanoate

A mixture of Example 65H (7.22 g) in anhydrous ethanol (160 mL) was treated with 21% sodium ethoxide mixture in ethanol (0.336 mL). The reaction mixture was stirred at room temperature for 5 hours and was quenched by the addition of acetic acid (0.103 mL). The volatiles were removed, and the residue was partitioned between ethyl acetate and brine. The organic phase was washed with brine and concentrated. The residue was purified by silica gel chromatography (5-50% ethyl acetate in heptane) to provide the title compound. MS (ESI) m/z 376 (M+NH₄)⁺.

### Example 65J

### (R)-ethyl 2-((5-bromo-6-(4-fluorophenyl)thieno[2,3-d]pyrimidin-4-yl)oxy)-3-(5-formyl-2-((4-methoxybenzyl)oxy)phenyl)propanoate

A mixture of Example 65I (5.28 g) and Example 1D (5.32 g) was suspended in 160 mL of anhydrous tert-butanol under nitrogen. Cesium carbonate (16.32 g) was added, and the mixture was stirred at 65° C. for 5 hours. After cooling, the reaction mixture was partitioned between ethyl acetate and brine. The organic phase was washed with brine, and concentrated. The residue was purified by silica gel chromatography (10-60% ethyl acetate in heptane) to provide the title compound. MS (ESI) m/z 666 (M+H)⁺.

### Example 65K

### (2R)-ethyl 2-((5-((1S)-3-chloro-4-(1,3-dioxan-2-yl)-2-methylphenyl)-6-(4-fluorophenyl)thieno[2,3-d]pyrimidin-4-yl)oxy)-3-(5-formyl-2-((4-methoxybenzyl)oxy)phenyl)propanoate

A 250 mL round-bottom flask was charged with Example 65J (9.32 g), Example 1S (6.16 g), potassium phosphate (8.92 g), and bis(di-tert-butyl(4-dimethylaminophenyl)phosphine)dichloropalladium (II) (992 mg). The flask was purged with nitrogen, and tetrahydrofuran (100 mL) and water (25 mL) were added. The reaction mixture was purged with nitrogen again and stirred at room temperature overnight. The reaction mixture was partitioned between ethyl acetate and brine. The organic phase was washed with brine, and concentrated. The residue was purified by silica gel chromatography (10-60% ethyl acetate in heptane) to provide the title compound. MS (ESI) m/z 797 (M+H)⁺.

### Example 65L

### ethyl (7R,20S)-18-chloro-1-(4-fluorophenyl)-10-[(4-methoxyphenyl)methoxy]-19-methyl-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylate

To a mixture of Example 65K (8.8 g) in a mixture of anhydrous dichloromethane (100 mL) and acetic acid (20 mL) was added 2-(4-methylpiperazin-1-yl)ethanamine (3.16 g). The mixture was stirred at room temperature for 1 hour before sodium triacetoxyborohydride (7.02 g) was added. The reaction mixture was stirred at room temperature overnight. The volatiles were removed by rotary evaporation, and the residue was dissolved in tetrahydrofuran (45 mL) and water (7.5 mL). The mixture was cooled to 0° C., and trifluoracetic acid (45 mL) was added. After the addition, the cooling bath was removed, and the mixture was stirred at room temperature for 4 hours. The mixture was diluted with ethyl acetate. The mixture was washed with a pre-cooled diluted sodium hydroxide mixture (contained about 60 mL of 50% sodium hydroxide, pH 10) and brine. The organic phase was concentrated. The residual intermediate was dissolved in anhydrous dichloromethane (100 mL). Anhydrous magnesium sulfate (25 g) was added. The mixture was stirred at room temperature overnight before sodium triacetoxyborohydride (7.02 g) was added. The reaction mixture was stirred at room temperature for 4 hours. The mixture was filtered, and the filtrate was directly purified by silica gel chromatography (0-20% methanol containing 3% ammonium hydroxide in dichloromethane) to provide the title compound. MS (ESI) m/z 850 (M+H)⁺.

### Example 65M

### ethyl (7R,20S)-18-chloro-1-(4-fluorophenyl)-10-hydroxy-19-methyl-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylate

Example 65L (2.9 g) was dissolved in anhydrous trifluoracetic acid (60 mL), and the mixture was heated at 45° C. for 1 hour. Anhydrous toluene (60 mL) was added, and the mixture was concentrated. The residue was concentrated with toluene again and dried under vacuum for 2 hours. Anhydrous ethanol (100 mL) was added, and the mixture was stirred at room temperature over a weekend. The volatiles were removed, and the residue was treated with triethylamine (2.5 mL) and loaded onto a silica gel column. The column was eluted with 0-20% methanol containing 3% ammonium hydroxide in dichloromethane to provide the title compound. MS (ESI) m/z 731 (M+H)⁺.

### Example 65N

### ethyl (7R,20S)-18-chloro-1-(4-fluorophenyl)-10-({2-[2-(2-methoxyethoxy)phenyl]pyrimidin-4-yl}methoxy)-19-methyl-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylate

A mixture of Example 65M (50 mg), Example 65E (38.2 mg), and cesium carbonate (89 mg) in anhydrous N,N-dimethylformamide (5 mL) was stirred at room temperature overnight. The reaction mixture was partitioned between ethyl acetate and brine. The organic phase was washed with brine, and concentrated. The residue was purified by silica gel chromatography (0-20% methanol containing 3% ammonium hydroxide in dichloromethane) to provide the title compound. MS (ESI) m/z 972 (M+H)⁺.

### Example 65O

### (7R,20S)-18-chloro-1-(4-fluorophenyl)-10-({2-[2-(2-methoxyethoxy)phenyl]pyrimidin-4-yl}methoxy)-19-methyl-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylic acid

To a mixture of Example 65N (45 mg) in tetrahydrofuran (1.5 mL) was added a mixture of lithium hydroxide monohydrate (4 mg) in water (1.5 mL) and methanol (1.5 mL). The mixture was stirred at room temperature for 2 days before trifluoracetic acid (0.04 mL) was added. The mixture was concentrated. The residue was purified by reverse-phase HPLC (Zorbax, C-18, 250×50 mm column, mobile phase A: 0.1% trifluoracetic acid in water; B: 0.1% trifluoracetic acid in CH₃CN; 0-70% gradient). Product-containing fractions were lyophilized to provide the title compound. ¹H NMR (400 MHz, dimethyl sulfoxide-d₆) δ ppm 8.71-8.61 (m, 3H), 7.61-7.52 (m, 3H), 7.50-7.41 (m, 2H), 7.33-7.00 (m, 12H), 6.84 (dd, 2H), 6.49 (s, 2H), 5.96 (dd, 2H), 5.19 (d, 1H), 5.15-5.04 (m, 2H), 4.37 (q, 4H), 4.19 (s, 2H), 4.11 (q, 3H), 3.23-2.92 (m, 4H), 2.79 (d, 6H), 1.74 (s, 3H). MS (ESI) m/z 944 (M+H)⁺.

### Example 66

### 18-chloro-1-(4-fluorophenyl)-19-methyl-15-[2-(4-methylpiperazin-1-yl)ethyl]-10-{[2-(3-methylpyridin-4-yl)pyrimidin-4-yl]methoxy}-7,8,15,16-tetrahydro-14H-17,20-etheno-13,9-(metheno)-6-oxa-2-thia-3,5,15-triazacyclooctadeca[1,2,3-cd]indene-7-carboxylic acid

### Example 66A

### 3-methylisonicotinonitrile

To a mixture of 3-chloroisonicotinonitrile (50 g) in toluene (1.5 L) was added K₃PO₄ (306 g), and the mixture was stirred for 10 minutes at 25° C. Methylboronic acid (32.4 g) and tricyclohexylphosphine (10.12 g) were added. After 5 minutes, 150 mL of water was added, and the mixture was stirred for 5 minutes at 25° C. Diacetoxypalladium (2.431 g) was added under a nitrogen atmosphere. The resulting mixture was stirred for 10 hours at 100° C. Eleven additional reactions were set up as described above. After cooling to 20° C., all twelve reaction mixtures were combined. 5 L of water was added to the mixture, and the layers were separated. The organic phase was dried over sodium sulfate, filtered and concentrated under reduced pressure to give a residue, which was purified by silica gel chromatography using 1-20% ethyl acetate in heptanes as the eluent to provide the title compound. ¹H NMR (400 MHz, chloroform-d) δ ppm 8.68 (s, 1H), 8.60 (d, 1H), 7.46 (d, 1H), 2.56 (s, 3H).

### Example 66B

### 3-methylisonicotinimidamide

To a suspension of ammonia hydrochloride (22.64 g) in toluene (500 mL) was added trimethylaluminum (211.5 mL) (2 M mixture in toluene) dropwise at 0° C. over 30 minutes (a lot of bubbles formed, at the end of addition the suspension almost became a mixture). After the addition, the mixture was stirred at 25° C. until there was no further evolution of gas. Example 66A (25 g) was added in portions. The resulting mixture was heated at 100° C. (internal temperature) for 12 hours. After cooling to 20° C., methanol (1.5 L) was added to the mixture dropwise. After stirring for 30 minutes, the mixture was filtered. The filtrate was concentrated under reduced pressure, and the residue was triturated with dichloromethane (600 mL) and filtered to provide the title compound. ¹H NMR (400 MHz, dimethyl sulfoxide-d₆) δ ppm 9.81-9.20 (m, 4H), 8.69-8.57 (m, 2H), 7.50 (d, 1H), 2.36 (s, 3H).

### Example 66C

### 4-(dimethoxymethyl)-2-(3-methylpyridin-4-yl)pyrimidine

To a mixture of Example 66B (50 g) in methanol (500 mL) was added (E)-4-(dimethylamino)-1,1-dimethoxybut-3-en-2-one (50.5 g) and sodium methanolate (26.8 g). The mixture was stirred at 75° C. for 12 hours. After cooling to 25° C., the reaction mixture was concentrated under reduced pressure. The residue was diluted with water (500 mL) and extracted with dichloromethane (3×400 mL). The combined organic layers were dried over sodium sulfate, filtered and concentrated under reduced pressure. The residue was purified by silica gel chromatography, eluting with petroleum ether and ethyl acetate (100/1 to 5/1) to provide the title compound. ¹H NMR (400 MHz, chloroform-d) δ ppm 8.92 (d, 1H), 8.57 (d, 2H), 7.79 (d, 1H), 7.54 (d, 1H), 5.36-5.32 (m, 1H), 3.47 (s, 6H), 2.57 (s, 3H).

### Example 66D

### (2-(3-methylpyridin-4-yl)pyrimidin-4-yl)methanol

To a mixture of Example 66C (40 g) in 1,4-dioxane (280 mL) was added 4N aqueous HCl mixture (280 mL) at 25° C. The mixture was stirred at 50° C. for 12 hours. After cooling to 0° C., a mixture of sodium hydroxide (44.8 g) in water (200 mL) was added dropwise at 0° C. The mixture was adjusted to pH 8 with 10% aqueous potassium carbonate (50 mL). Sodium tetrahydroborate (12.34 g) was added portionwise, and the mixture was stirred for 30 minutes at 0° C. After completion of the reaction, all five reaction mixtures were combined, diluted with water (2 L), and extracted with dichloromethane (3×1 L). The combined organic phases were dried over sodium sulfate, filtered and concentrated under reduced pressure. The crude product was purified by silica gel chromatography eluting with dichloromethane and methanol (1000/1 to 20/1) to provide the title compound. ¹H NMR (400 MHz, (chloroform-d) δ ppm 8.85 (d, 1H), 8.60-8.50 (m, 2H), 7.77 (d, 1H), 7.40 (d, 1H), 4.87 (s, 2H), 4.14 (br s, 1H), 2.56 (s, 3H).

### Example 66E

### 4-(chloromethyl)-2-(3-methylpyridin-4-yl)pyrimidine

To a mixture of Example 66D (300 mg) in anhydrous dichloromethane (20 mL) was added triphenylphosphine (508 mg) at 0° C. The mixture was stirred at 0° C. for 45 minutes, and N-chlorosuccinimide (219 mg) was added. The reaction mixture was allowed to warm to room temperature for 3 hours. The mixture was directly loaded onto a silica gel column which was eluted with 20-70% ethyl acetate in heptane to provide the title compound. The product was not stable at room temperature, and was immediately used in the next step. MS (DCI) m/z 220 (M+H)⁺.

### Example 66F

### ethyl (7R,20S)-18-chloro-1-(4-fluorophenyl)-19-methyl-15-[2-(4-methylpiperazin-1-yl)ethyl]-10-{[2-(3-methylpyridin-4-yl)pyrimidin-4-yl]methoxy}-7,8,15,16-tetrahydro-14H-17,20-etheno-13,9-(metheno)-6-oxa-2-thia-3,5,15-triazacyclooctadeca[1,2,3-cd]indene-7-carboxylate

The title compound was prepared as described in Example 65N, substituting Example 66E for Example 65E. MS (ESI) m/z 914 (M+H)⁺.

### Example 66G

### (7R,20S)-18-chloro-1-(4-fluorophenyl)-19-methyl-15-[2-(4-methylpiperazin-1-yl)ethyl]-10-{[2-(3-methylpyridin-4-yl)pyrimidin-4-yl]methoxy}-7,8,15,16-tetrahydro-14H-17,20-etheno-13,9-(metheno)-6-oxa-2-thia-3,5,15-triazacyclooctadeca[1,2,3-cd]indene-7-carboxylic acid

The title compound was prepared as described in Example 65O, substituting Example 66F for Example 65N. ¹H NMR (400 MHz, dimethyl sulfoxide-d₆) δ ppm 8.84 (d, 1H), 8.66-8.57 (m, 3H), 7.82 (d, 1H), 7.51 (d, 1H), 7.41 (d, 1H), 7.31-7.11 (m, 6H), 6.87 (d, 1H), 6.51 (d, 1H), 5.92 (dd, 2H), 5.26 (d, 2H), 5.09 (d, 2H), 4.42-4.21 (m, 3H), 4.20-4.08 (m, 2H), 2.97 (s, 12H), 2.79 (s, 5H), 1.72 (s, 3H). MS (ESI) m/z 885 (M+H)⁺.

### Example 67

### (7R,21S)-19-chloro-1-(4-fluorophenyl)-10-{[2-(2-methoxyphenyl)pyrimidin-4-yl]methoxy}-20-methyl-15-oxo-16-[2-(piperazin-1-yl)ethyl]-7,8,14,15,16,17-hexahydro-18,21-etheno-13,9-(metheno)-6-oxa-2-thia-3,5,16-triazacyclononadeca[1,2,3-cd]indene-7-carboxylic acid

To a mixture of Example 59G (30 mg) in tetrahydrofuran (260 μL) and methanol (260 μL) was added a mixture of lithium hydroxide (8.4 mg) in water (260 μL), and the reaction mixture was allowed to stir overnight. The reaction mixture was quenched with trifluoroacetic acid (45 μL) and was diluted with dimethyl sulfoxide (600 μL). The mixture was purified by reverse-phase HPLC Gilson PLC 2020 using a Luna column (250×50 mm, 10 mm) (5-70% over 30 minutes with acetonitrile in water containing 0.1% trifluoroacetic acid) to provide the title compound after lyophilization. ¹H NMR (400 MHz, dimethyl sulfoxide-d₆) δ ppm 9.18 (br s, 1H), 8.70 (d, 1H), 8.52 (s, 1H), 7.55-7.41 (m, 3H), 7.30-6.98 (m, 10H), 6.77 (d, 1H), 4.99-4.71 (m, 4H), 4.49 (d, 1H), 4.45-4.32 (m, 1H), 3.85 (d, 1H), 3.75 (s, 3H), 3.49-3.10 (m, 12H), 1.83 (br s, 3H). MS (ESI) m/z 914.3 (M+H)⁺.

### Example 68

### (7R,16R,21S)-19-chloro-1-(4-fluorophenyl)-10-{[2-(2-methoxyphenyl)pyrimidin-4-yl]methoxy}-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid

### Example 68A

### (R)-ethyl 2-acetoxy-3-(5-((tert-butyldimethylsilyl)oxy)-2-((2-(2-methoxyphenyl)pyrimidin-4-yl)methoxy)phenyl)propanoate

To an oven dried 500 mL round bottom flask was added Example 16D (8 g), triphenylphosphine (13.71 g), Example 1G (6.78 g) and tetrahydrofuran (105 mL). The reaction flask was cooled in an ice bath. Solid (E)-N,N,N′,N′-tetramethyldiazene-1,2-dicarboxamide (9 g) was added and the reaction mixture was allowed to warm up to ambient temperature and was stirred overnight. After ˜2 minutes, a precipitate was observed. After 48 hours, thin-layer chromatography indicated complete consumption of starting material. The reaction mixture was concentrated. Ethyl acetate (50 mL) was added to the material and the mixture was stirred for about 30 minutes and filtered. The filtrate was concentrated and purified by silica gel chromatography on a Grace Reveleris system using a 120 g silica column with 0-25% ethyl acetate/heptanes. Fractions containing desired product were combined and concentrated to obtain the title compound. ¹H NMR (400 MHz, dimethyl sulfoxide-d₆) δ ppm 8.92 (d, 1H), 7.59-7.50 (m, 2H), 7.46 (ddd, 1H), 7.15 (dd, 1H), 7.05 (td, 1H), 6.95 (d, 1H), 6.77-6.68 (m, 2H), 5.25-5.11 (m, 3H), 4.07 (qd, 2H), 3.76 (s, 3H), 3.26 (dd, 2H), 3.05 (dd, 1H), 1.99 (s, 3H), 1.10 (t, 3H), 0.93 (s, 9H), 0.15 (s, 6H). MS (ESI) m/z 581.4 (M+H)⁺.

### Example 68B

### (R)-ethyl 3-(5-((tert-butyldimethylsilyl)oxy)-2-((2-(2-methoxyphenyl)pyrimidin-4-yl)methoxy)phenyl)-2-hydroxypropanoate

To a mixture of Example 68A (12.60 g) in anhydrous ethanol (220 mL) was added anhydrous potassium carbonate (11.99 g), and the mixture was stirred at room temperature and monitored by LC/MS. After 1 hour, LC/MS showed complete consumption of starting material with a major peak consistent with desired product. The mixture was filtered, and the material was rinsed with ethyl acetate. The filtrate was concentrated under reduced pressure. To the residue was added water (100 mL) and ethyl acetate (100 mL). The layers were separated, and the aqueous layer was extracted with three portions of ethyl acetate. The combined organic layers were dried over anhydrous sodium sulfate, filtered and concentrated. The crude product was used in the next step without further purification. LC/MS (APCI) m/z 539.2 (M+H)⁺.

### Example 68C

### (R)-ethyl 2-((5-bromo-6-(4-fluorophenyl)thieno[2,3-d]pyrimidin-4-yl)oxy)-3-(5-((tert-butyldimethylsilyl)oxy)-2-((2-(2-methoxyphenyl)pyrimidin-4-yl)methoxy)phenyl)propanoate

To a mixture of Example 68B (11.10 g) and Example 1D (7.08 g) was added anhydrous cesium carbonate (20.14 g). The mixture was evacuated and backfilled with nitrogen, and anhydrous tert-butanol (180 mL) was added. The mixture was stirred at 65° C. for 5 hours and was concentrated under reduced pressure. The residue was diluted with ethyl acetate, washed with water and brine, dried over anhydrous sodium sulfate, filtered, and concentrated. The crude material was purified by silica gel chromatography on an AnaLogix IntelliFlash²⁸⁰ system (10-70% ethyl acetate/heptanes, linear gradient) to provide the title compound. LC/MS (APCI) m/z 847.1 (M+H)⁺.

### Example 68D

### (2R)-ethyl 2-((5-((1S)-4-(((R)-1-(bis(4-methoxyphenyl)(phenyl)methoxy)-3-(4-methylpiperazin-1-yl)propan-2-yl)oxy)-3-chloro-2-methylphenyl)-6-(4-fluorophenyl)thieno[2,3-d]pyrimidin-4-yl)oxy)-3-(5-((tert-butyldimethylsilyl)oxy)-2-((2-(2-methoxyphenyl)pyrimidin-4-yl)methoxy)phenyl)propanoate

A mixture of Example 68C (5.580 g), Example 64K (7.34 g), bis(di-tert-butyl(4-dimethylaminophenyl)phosphine)dichloropalladium(II) (0.701 g) and cesium carbonate (6.45 g) was evacuated and backfilled with nitrogen twice. Freshly degassed tetrahydrofuran (50 mL) followed by water (12.50 mL) was introduced, and the reaction mixture was evacuated and backfilled with nitrogen twice again with stirring. The mixture was stirred at 40° C. for 1 day. The reaction mixture was partitioned between ethyl acetate and water. The organic layer was collected, and the aqueous layer was extracted with two portions of ethyl acetate. The organics were combined, dried over anhydrous magnesium sulfate, filtered and concentrated. The residue was purified by silica gel flash chromatography on an AnaLogix IntelliFlash²⁸⁰ system (solvent A=2:1 ethyl acetate:ethanol; solvent B=heptane; 20-100% A to B) to provide the title compound. LC/MS (APCI) m/z 1366.6 (M+H)⁺.

### Example 68E

### (2R)-ethyl 3-(5-((tert-butyldimethylsilyl)oxy)-2-((2-(2-methoxyphenyl)pyrimidin-4-yl)methoxy)phenyl)-2-((5-((1S)-3-chloro-4-(((R)-1-hydroxy-3-(4-methylpiperazin-1-yl)propan-2-yl)oxy)-2-methylphenyl)-6-(4-fluorophenyl)thieno[2,3-d]pyrimidin-4-yl)oxy)propanoate

Example 68D (8.62 g) was dissolved in dichloromethane (20 mL) and methanol (20 mL). To the resulting stirring mixture was added formic acid (13.94 g), and the mixture was stirred at ambient temperature for 1 hour. The mixture was treated with saturated aqueous sodium bicarbonate until neutralized. The mixture was diluted with 150 mL of water and was extracted with three portions of ethyl acetate. The organic extracts were combined, dried over anhydrous sodium sulfate, filtered and concentrated. The residue was purified by silica gel chromatography on an AnaLogix IntelliFlash²⁸⁰ system (solvent A=2:1 methanol:water; solvent B=ethyl acetate, 4-30% A to B) to provide the title compound. LC/MS (APCI) m/z 1063.0 (M+H)⁺.

### Example 68F

### (2R)-ethyl 2-((5-((1S)-3-chloro-4-(((R)-1-hydroxy-3-(4-methylpiperazin-1-yl)propan-2-yl)oxy)-2-methylphenyl)-6-(4-fluorophenyl)thieno[2,3-d]pyrimidin-4-yl)oxy)-3-(5-hydroxy-2-((2-(2-methoxyphenyl)pyrimidin-4-yl)methoxy)phenyl)propanoate

Example 68E (4500 mg) was treated with tetrabutylammonium fluoride (25 mL, 1 M in tetrahydrofuran). The reaction mixture was stirred at ambient temperature for 30 minutes and was concentrated under reduced pressure. The residue was purified by silica gel chromatography on an AnaLogix IntelliFlash²⁸⁰ system (eluting, solvent A=2:1 methanol:water; solvent B=ethyl acetate; 2-30% A/B) to obtain the title compound. LC/MS (APCI) m/z 949.2 (M+H)⁺.

### Example 68G

### ethyl (7R,16R,21S)-19-chloro-1-(4-fluorophenyl)-10-{[2-(2-methoxyphenyl)pyrimidin-4-yl]methoxy}-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

A mixture of Example 68F (2600 mg), triphenylphosphine (1006 mg) and N,N,N′,N′-tetramethylazodicarboxamide (660 mg) was evacuated and backfilled with nitrogen twice. Toluene (150 mL) was added, and the the vessel was evacuated and backfilled with nitrogen. The mixture was stirred at 50° C. for 16 hours. The reaction mixture was concentrated under reduced pressure and was purified by silica gel chromatography on an AnaLogix IntelliFlash²⁸⁰ system (0-7% methanol in dichloromethane) to provide the title compound as a mixture of isomers. MS (ESI) m/z 931.3 (M+H)⁺.

### Example 68H

### (7R,16R,21S)-19-chloro-1-(4-fluorophenyl)-10-{[2-(2-methoxyphenyl)pyrimidin-4-yl]methoxy}-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid

To a mixture of Example 68F (1390 mg) in tetrahydrofuran (15 mL) and methanol (15 mL) was added lithium hydroxide (1.0 M in water) (20.15 mL). The mixture was stirred at ambient temperature for 1 day. To the mixture was added N,N-dimethylformamide (1 mL), and the mixture was acidified with trifluoroacetic acid. The mixture was purified on a Gilson RP HPLC (Zorbax, C-18, 250×21.2 mm column, 5 to 90% acetonitrile in water (0.1% trifluoroacetic acid)) to provide the title compound after lyophilization. Example 63 and Example 73 were also isolated from this reaction mixture. ¹H NMR (501 MHz, dimethyl sulfoxide-d₆) δ ppm 8.87 (d, 1H), 8.73 (s, 1H), 7.56-7.50 (m, 2H), 7.49-7.43 (m, 1H), 7.27-7.13 (m, 6H), 7.06 (t, 1H), 6.93 (d, 1H), 6.88 (d, 1H), 6.71 (dd, 1H), 6.29 (dd, 1H), 5.80 (d, 1H), 5.24-5.06 (m, 3H), 4.44-4.30 (m, 1H), 4.02-3.91 (m, 1H), 3.83 (dd, 1H), 3.77 (s, 3H), 3.72-3.00 (m, 9H), 2.99-2.83 (m, 2H), 2.79 (s, 3H), 2.18 (s, 3H). MS (ESI) m/z 903.4 (M+H)⁺.

### Example 69

### (7R,20R)-2,18-dichloro-1-(4-fluorophenyl)-10-{[2-(2-methoxyphenyl)pyrimidin-4-yl]methoxy}-19-methyl-15-[2-(4-methylpiperazin-1-yl)ethyl]-7,8,15,16-tetrahydro-14H-17,20-etheno-9,13-(metheno)-6-oxa-2a,5,15-triazacyclooctadeca[1,2,3-cd]indene-7-carboxylic acid

### Example 69A

### methyl 4-(4-fluorophenyl)-1H-pyrrole-2-carboxylate

To a 3 L three-necked flask with an internal temperature probe, a condenser and a stir bar was added K₃PO₄ (94 g), (4-fluorophenyl)boronic acid (49.4 g), methyl 4-bromo-1H-pyrrole-2-carboxylate (60 g), water (60 mL) and toluene (490 mL). The mixture was sparged with nitrogen gas for 30 minutes. In a separate 250 mL flask, Pd₂(dba)₃ (tris(dibenzylideneacetone)dipalladium(0), 2.69 g) and XPhos (2-dicyclohexylphosphino-2′,4′,6′-triisopropylbiphenyl, 5.89 g) were added followed by 50 mL of toluene that had been sparged with nitrogen gas for 30 minutes. The mixture was heated under nitrogen gas to 70° C. and was stirred for 15 minutes. The contents of the 250 mL flask were transferred to the 3 L flask using a cannula, and the 3 L flask was heated to 85° C. and stirred overnight under nitrogen gas. The next morning the reaction mixture was cooled to ambient temperature. As the reaction cooled, the homogeneous reaction mixture turned into a slurry. The slurry was poured into a 2 L separatory funnel. The reaction vessel was washed with water (400 mL) and ethyl acetate (400 mL). The washings were poured into the separatory funnel, and layers were separated. The aqueous layer was extracted once with 200 mL ethyl acetate. The combined organic layers were dried (brine and magnesium sulfate), filtered and concentrated. To the residue was added 10% ethyl acetate/heptanes (200 mL), and the mixture was stirred for 20 minutes and filtered on a Buchner funnel. The material in the funnel was washed with 10% ethyl acetate/heptanes (800 mL) and dried. The process was repeated on the material obtained after concentrating the filtrate, and the material was combined to provide the title compound. ¹H NMR (400 MHz, dimethyl sulfoxide-d₆) δ ppm 12.07 (bs, 1H), 7.68-7.61 (m, 2H), 7.49 (d, 1H), 7.17 (d, 1H), 7.16-7.10 (m, 2H), 3.78 (s, 3H). MS (ESI) m/z 218.0 (M−H)⁺.

### Example 69B

### 4-(4-fluorophenyl)-1H-pyrrole-2-carboxamide

To a 250 mL Parr stainless steel reactor was added Example 69A (15.25 g) followed by ammonium hydroxide mixture (28% w/w, 318 mL). The reactor was sealed heated at 100° C. with stirring set at 1200 RPM. The reaction mixture was stopped after 4 hours. The reaction mixture was allowed to cool to ambient temperature and filtered to isolate a material that was dried in a vacuum oven (30 mbar, 50° C.) overnight to provide the title compound. ¹H NMR (500 MHz, dimethyl sulfoxide-d₆) δ ppm 11.58 (bs, 1H), 7.62-7.46 (m, 2H), 7.30 (dd, 1H), 7.18-7.13 (m, 2H), 7.11 (dd, 1H), 7.01 (bs, 1H). MS (ESI) m/z 205.1 (M+H)⁺.

### Example 69C

### 7-(4-fluorophenyl)pyrrolo[1,2-a]pyrazin-1-ol

To a 2 L three-necked round bottom flask equipped with a stir bar, an internal temperature probe and a reflux condenser was added Example 69B (35 g), N,N-dimethylformamide (400 mL), cesium carbonate (84 g) and 2-bromo-1,1-dimethoxyethane (30.4 mL). The reaction mixture was heated to 90° C. and was stirred overnight. The next morning, the reaction mixture was cooled to ambient temperature, diluted with ethyl acetate (400 mL) and poured into a separatory funnel containing 400 mL water and 100 mL ammonium hydroxide. The two layers were separated. The aqueous layer was extracted with ethyl acetate (2×150 mL). The combined organic layers were washed with water (4×100 mL) and brine, dried over magnesium sulfate, filtered, and concentrated to obtain crude product. The material was dissolved in dichloromethane (300 mL) and hydrogen chloride (concentrated, 14.25 mL) was added in one portion. The reaction mixture was stirred vigorously at ambient temperature. After 10 minutes, a material started appearing. After 3 hours, the mixture was filtered, and the material was washed with dichloromethane (2×100 mL). The filtrate was concentrated to obtain a slurry to which was added 100 mL of 1:1 ethyl acetate/heptanes. A material precipitated which was filtered and the material in the funnel was washed with 200 mL of 1:1 ethyl acetate/heptanes. The material was combined and placed in a vacuum oven (30 mbar, 50° C.) overnight to obtain the title compound. ¹H NMR (400 MHz, dimethyl sulfoxide-d₆) δ ppm 10.48 (bs, 1H), 7.86 (d, 1H), 7.75-7.67 (m, 2H), 7.28 (d, 1H), 7.26 (d, 1H), 7.24-7.17 (m, 2H), 6.59 (t, 1H). MS (ESI) m/z 229.0 (M+H)⁺.

### Example 69D

### 1-chloro-7-(4-fluorophenyl)pyrrolo[1,2-a]pyrazine

To a 1 L, three-necked round bottom flask equipped with a stir bar, an internal temperature probe and a reflux condenser was added Example 69C (20 g), toluene (400 mL) and N-ethyl-N-isopropylpropan-2-amine (18.32 mL). Neat phosphoryl trichloride (9.80 mL) was added dropwise. During the addition, fumes were observed in the flask, and the internal temperature rose by 1° C. The reaction flask was heated to 111° C. and was stirred overnight. The next morning the reaction mixture was cooled to ambient temperature and was poured over aqueous saturated sodium bicarbonate and extracted with ethyl acetate. The crude material was purified on a silica plug (5″ wide, 2″ high), with 10-25% ethyl acetate/heptanes elution gradient. Fractions containing the desired product were combined, concentrated and dried under vacuum to obtain the title compound. ¹H NMR (501 MHz, dimethyl sulfoxide-d₆) δ ppm 8.32 (d, 1H), 8.29 (dd1H), 7.88-7.83 (m, 2H), 7.36 (d, 1H), 7.29 (dd, 1H), 7.29-7.24 (m, 2H). MS (ESI) m/z 247.1 (M+H)⁺.

### Example 69E

### 1,6-dichloro-7-(4-fluorophenyl)pyrrolo[1,2-a]pyrazine

To a mixture of Example 69D (6 g) in tetrahydrofuran (300 mL) was added N-chlorosuccinimide (16.2 g). The mixture was stirred at 50° C. for 12 hours. The reaction mixture was cooled to room temperature, diluted with ethyl acetate (200 mL) and washed with water (2×200 mL). The organic layer was dried over sodium sulfate, filtered and concentrated. The residue was purified by column chromatography on silica gel, eluting with 50:1-10:1 petroleum ether:ethyl acetate, to provide the title compound. MS (ESI) m/z 280.8 (M+H)⁺.

### Example 69F

### 1,6-dichloro-7-(4-fluorophenyl)-8-iodopyrrolo[1,2-a]pyrazine

To a mixture of Example 69E (5 g) in N,N-dimethylformamide (60 mL) was added N-iodosuccinimide (12.01 g). The mixture was stirred at 50° C. for 12 hours. The reaction mixture was cooled to room temperature, diluted with ethyl acetate (200 mL), and washed with aqueous sodium thiosulfate mixture (2×150 mL) and water (2×200 mL). The organic layer was dried over sodium sulfate, filtered, and concentrated. The residue was purified by column chromatography on silica gel, eluting with 50:1-10:1 petroleum ether:ethyl acetate, to provide the title compound. ¹H NMR (400 MHz, dimethyl sulfoxide-d₆) δ ppm 8.36-8.24 (m, 1H), 7.60-7.51 (m, 1H), 7.51-7.42 (m, 2H) and 7.41-7.32 (m, 2H). MS (ESI) m/z 406.8 (M+H)⁺.

### Example 69G

### 6-chloro-1-fluoro-7-(4-fluorophenyl)-8-iodopyrrolo[1,2-a]pyrazine

To a mixture of Example 69F (3.6 g) in N,N-dimethylformamide (27 mL) was added tetramethylammonium fluoride (1.63 g), and the reaction mixture was allowed to stir overnight. The reaction mixture was diluted with ethyl acetate, washed with water and brine, dried over sodium sulfate, filtered and concentrated. The crude material was purified by normal phase MPLC on a Teledyne Isco Combiflash Rf+ (0-15% ethyl acetate in heptanes) to provide the title compound. MS (ESI) m/z 390.9 (M+H)⁺.

### Example 69H

### (R)-ethyl 2-((6-chloro-7-(4-fluorophenyl)-8-iodopyrrolo[1,2-a]pyrazin-1-yl)oxy)-3-(5-formyl-2-((2-(2-methoxyphenyl)pyrimidin-4-yl)methoxy)phenyl)propanoate

To a mixture of Example 69G (164 mg) and Example 10 (175 mg) in tert-butanol (7.1 mL) and N,N-dimethylformamide (0.900 mL) was added cesium carbonate (392 mg), and the reaction mixture was warmed to 38° C. overnight. The reaction mixture was cooled, concentrated, diluted with water and extracted with ethyl acetate three times. The combined organic layers were dried over sodium sulfate, filtered and concentrated. The residue was purified by normal phase MPLC (20-90% ethyl acetate in heptanes) followed by reverse-phase HPLC Gilson PLC 2020 using a Luna column (250×50 mm, 10 mm) (25-100% acetonitrile in water containing 0.1% trifluoroacetic acid) to provide the title compound. MS (ESI) m/z 807.0 (M+H)⁺.

### Example 69I

### (2R)-ethyl 2-((6-chloro-8-((3-chloro-4-(1,3-dioxan-2-yl)-2-methylphenyl)-7-(4-fluorophenyl)pyrrolo[1,2-a]pyrazin-1-yl)oxy)-3-(5-formyl-2-((2-(2-methoxyphenyl)pyrimidin-4-yl)methoxy)phenyl)propanoate

Example 69H (163 mg), Example 1S (82 mg), bis(di-tert-butyl(4-dimethylaminophenyl)phosphine)dichloropalladium (14.3 mg) and cesium carbonate (197 mg) were combined in a vial and purged with nitrogen three times. Tetrahydrofuran (1.5 mL) and water (470 μL) were added, and the reaction mixture was warmed to 65° C. After 3 minutes, the reaction mixture was cooled to room temperature and was allowed to stir overnight. 1-Pyrrolidinecarbodithioic acid ammonium salt (3.3 mg) was added, and the reaction mixture was stirred for 30 minutes. The reaction mixture was filtered over diatomaceous earth, washing with ethyl acetate. The filtrate was diluted with brine and was extracted with ethyl acetate three times. The combined organic layers were dried over sodium sulfate, filtered and concentrated. The crude residue was purified by normal phase MPLC on a Teledyne Isco Combiflash Rf+ (20-100% ethyl acetate in heptanes) to give a residue that was further purified by normal phase MPLC on a Teledyne Isco Combiflash Rf+ (0-30 ethyl acetate in dichloromethane) to provide the title compound. MS (ESI) m/z 891.2 (M+H)⁺.

### Example 69J

### (2R)-ethyl 2-((6-chloro-8-((3-chloro-4-formyl-2-methylphenyl)-7-(4-fluorophenyl)pyrrolo[1,2-a]pyrazin-1-yl)oxy)-3-(2-((2-(2-methoxyphenyl)pyrimidin-4-yl)methoxy)-5-(((2-(4-methylpiperazin-1-yl)ethyl)amino)methyl)phenyl)propanoate

To a mixture of 2-(4-methylpiperazin-1-yl)ethanamine (7.2 mg) and Example 69I (41 mg) in dichloromethane was added acetic acid (10.5 μL), and the reaction mixture was allowed to stir for 30 minutes. Sodium triacetoxyborohydride (19.5 mg) was added, and the reaction mixture was allowed to stir for 1 hour. The reaction mixture was diluted with ethyl acetate and water. The aqueous layer was extracted three times with ethyl acetate. The combined organic layers were washed with saturated sodium bicarbonate and brine, dried over sodium sulfate, filtered and concentrated to give a crude product that was used without further purification. A mixture of tetrahydrofuran (1 mL), trifluoroacetic acid (1 mL) and water (333 μL) was added to the crude material, and the mixture was allowed to stir for 1 hour. The reaction mixture was slowly quenched with saturated sodium bicarbonate mixture and was extracted with ethyl acetate three times. The combined organic layers were dried over sodium sulfate, filtered and concentrated. The crude residue was purified by reverse-phase HPLC Gilson PLC 2020 using a Luna column (250×50 mm, 10 mm) (5-80% acetonitrile in water containing 0.1% trifluoroacetic acid). The appropriate fractions were combined, neutralized with saturated sodium bicarbonate, extracted with dichloromethane, dried over sodium sulfate, filtered and concentrated to provide the title compound. MS (ESI) m/z 960.3 (M+H)⁺.

### Example 69K

### ethyl (7R,20R)-2,18-dichloro-1-(4-fluorophenyl)-10-{[2-(2-methoxyphenyl)pyrimidin-4-yl]methoxy}-19-methyl-15-[2-(4-methylpiperazin-1-yl)ethyl]-7,8,15,16-tetrahydro-14H-17,20-etheno-9,13-(metheno)-6-oxa-2a,5,15-triazacyclooctadeca[1,2,3-cd]indene-7-carboxylate

To a mixture of Example 69J (28 mg) in dichloromethane (2.9 mL) was added anhydrous magnesium sulfate (250 mg), and the reaction mixture was allowed to stir for 1 hour. To the suspension was added sodium triacetoxyborohydride (18.5 mg), and the reaction mixture was stirred overnight. The reaction mixture was filtered over diatomaceous earth, diluted with saturated sodium bicarbonate and extracted with dichloromethane three times. The combined organic layers were dried over sodium sulfate, filtered and concentrated. The residue was purified by reverse-phase HPLC Gilson PLC 2020 using a Luna column (250×50 mm, 10 mm) (5-70% acetonitrile in water containing 0.1% trifluoroacetic acid) and lyophilized to provide the title compound. MS (ESI) m/z 944.3 (M+H)⁺.

### Example 69L

### ethyl (7R,20S)-2,18-dichloro-1-(4-fluorophenyl)-10-{[2-(2-methoxyphenyl)pyrimidin-4-yl]methoxy}-19-methyl-15-[2-(4-methylpiperazin-1-yl)ethyl]-7,8,15,16-tetrahydro-14H-17,20-etheno-9,13-(metheno)-6-oxa-2a,5,15-triazacyclooctadeca[1,2,3-cd]indene-7-carboxylate

The title compound was obtained as a minor product during the synthesis of Example 69K. MS (ESI) m/z 944.3 (M+H)⁺.

### Example 69M

### (7R,20R)-2,18-dichloro-1-(4-fluorophenyl)-10-{[2-(2-methoxyphenyl)pyrimidin-4-yl]methoxy}-19-methyl-15-[2-(4-methylpiperazin-1-yl)ethyl]-7,8,15,16-tetrahydro-14H-17,20-etheno-9,13-(metheno)-6-oxa-2a,5,15-triazacyclooctadeca[1,2,3-cd]indene-7-carboxylic acid

To a mixture of Example 69K (19.7 mg) in tetrahydrofuran (200 μL) and methanol (200 μL) was added a mixture of lithium hydroxide (7.3 mg), and the reaction mixture was allowed to stir overnight. The reaction mixture was quenched with trifluoroacetic acid (30 μL) and was purified by reverse-phase HPLC Gilson PLC 2020 using a Luna column (250×50 mm, 10 mm) (5-65% acetonitrile in water containing 0.1% trifluoroacetic acid) to provide the title compound after lyophilization. ¹H NMR (400 MHz, dimethyl sulfoxide-d₆) δ ppm 8.53 (d, 1H), 7.90 (d, 1H), 7.54-7.42 (m, 3H), 7.33-7.00 (m, 10H), 6.79 (d, 1H), 6.67 (br s, 1H), 5.80 (dd, 1H), 5.18 (d, 1H), 4.98 (d, 1H), 4.62-4.44 (m, 2H), 4.37-4.22 (m, 2H), 3.75 (s, 3H), 3.33-3.22 (m, 2H), 3.16-2.91 (m, 5H), 2.81 (s, 3H), 1.50 (s, 3H). MS (ESI) m/z 916.2 (M+H)⁺.

### Example 70

### (7R,20S)-10-[(1-butyl-1H-pyrazol-5-yl)methoxy]-18-chloro-1-(4-fluorophenyl)-19-methyl-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylic acid

### Example 70A

### 1-butyl-5-(chloromethyl)-1H-pyrazole

To a mixture of (1-butyl-1H-pyrazol-5-yl)methanol (500 mg) in anhydrous dichloromethane (20 mL) was added triphenylphosphine (1.1 g) at 0° C. The mixture was stirred at 0° C. for 45 minutes, and N-chlorosuccinimide (476 mg) was added. The reaction mixture was allowed to warm to room temperature overnight. The reaction mixture was directly loaded onto a silica gel column that was eluted with 20-60% ethyl acetate in heptane to provide the title compound. MS (DCI) m/z 173 (M+H)⁺.

### Example 70B

### ethyl (7R,20S)-10-[(1-butyl-1H-pyrazol-5-yl)methoxy]-18-chloro-1-(4-fluorophenyl)-19-methyl-15-[2-(4-methylpiperazin-1-yl)ethyl]-7,8,15,16-tetrahydro-14H-17,20-etheno-9,13-(metheno)-6-oxa-2-thia-3,5,15-triazacyclooctadeca[1,2,3-cd]indene-7-carboxylate

Example 70B was prepared according to the procedure described for Example 65N, substituting Example 70A for 65E. MS (APCI) m/z 866.24 (M+H)⁺.

### Example 70C

### (7R,20S)-10-[(1-butyl-1H-pyrazol-5-yl)methoxy]-18-chloro-1-(4-fluorophenyl)-19-methyl-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylic acid

Example 70C was prepared according to the procedure described for Example 65O, substituting Example 70B for Example 65N. ¹H NMR (400 MHz, dimethyl sulfoxide-d₆) δ ppm 8.68 (s, 1H), 7.51 (d, 2H), 7.36-7.28 (m, 2H), 7.28-7.18 (m, 3H), 7.14 (t, 2H), 6.96 (d, 1H), 6.49 (s, 1H), 6.13 (s, 1H), 5.73 (dd, 1H), 5.06 (d, 2H), 4.96 (d, 2H), 4.39-4.23 (m, 2H), 4.16 (s, 2H), 3.87 (td, 3H), 3.13-2.92 (m, 8H), 2.80 (s, 3H), 1.69 (s, 3H), 1.61 (p, 3H), 1.12 (h, 3H), 0.78 (t, 3H). MS (ESI) m/z 838 (M+H)⁺.

### Example 71

### (7R,20S)-18-chloro-1-(4-fluorophenyl)-19-methyl-15-[2-(4-methylpiperazin-1-yl)ethyl]-10-{[2-(3,3,3-trifluoropropoxy)pyrimidin-4-yl]methoxy}-7,8,15,16-tetrahydro-14H-17,20-etheno-13,9-(metheno)-6-oxa-2-thia-3,5,15-triazacyclooctadeca[1,2,3-cd]indene-7-carboxylic acid

### Example 71A

### 4-(chloromethyl)-2-(3,3,3-trifluoropropoxy)pyrimidine

To a mixture of Example 7E (400 mg) in anhydrous dichloromethane (20 mL) was added triphenylphosphine (614 mg) at 0° C. The mixture was stirred at 0° C. for 45 minutes, and N-chlorosuccinimide (264 mg) was added. The reaction mixture was allowed to warm to room temperature for 2 hours, and was directly loaded onto a silica gel column that was eluted with 10-50% ethyl acetate in heptane to provide the title compound. MS (DCI) m/z 257 (M+NH₄)⁺.

### Example 71B

### ethyl (7R,20S)-18-chloro-1-(4-fluorophenyl)-19-methyl-15-[2-(4-methylpiperazin-1-yl)ethyl]-10-{[2-(3,3,3-trifluoropropoxy)pyrimidin-4-yl]methoxy}-7,8,15,16-tetrahydro-14H-17,20-etheno-9,13-(metheno)-6-oxa-2-thia-3,5,15-triazacyclooctadeca[1,2,3-cd]indene-7-carboxylate

Example 71B was prepared according to the procedure described for Example 65N, substituting Example 71A for 65E. MS (APCI) m/z 934.21 (M+H)⁺.

### Example 71C

### (7R,20S)-18-chloro-1-(4-fluorophenyl)-19-methyl-15-[2-(4-methylpiperazin-1-yl)ethyl]-10-{[2-(3,3,3-trifluoropropoxy)pyrimidin-4-yl]methoxy}-7,8,15,16-tetrahydro-14H-17,20-etheno-13,9-(metheno)-6-oxa-2-thia-3,5,15-triazacyclooctadeca[1,2,3-cd]indene-7-carboxylic acid

Example 71C was prepared according to the procedure described for Example 65O, substituting Example 71B for Example 65N. ¹H NMR (400 MHz, dimethyl sulfoxide-d₆) δ ppm 8.65 (s, 1H), 8.41 (d, 1H), 7.51 (d, 2H), 7.32-7.10 (m, 5H), 6.95 (d, 1H), 6.79 (d, 1H), 6.48 (d, 1H), 5.91 (dd, 1H), 5.08 (t, 2H), 4.97 (d, 2H), 4.48 (t, 2H), 4.32 (t, 2H), 4.15 (s, 2H), 3.26-2.97 (m, 11H), 2.86-2.73 (m, 6H), 1.73 (s, 3H). MS (ESI) m/z 906 (M+H)⁺.

### Example 72

### (7R,20S)-2,18-dichloro-1-(4-fluorophenyl)-10-{[2-(2-methoxyphenyl)pyrimidin-4-yl]methoxy}-19-methyl-15-[2-(4-methylpiperazin-1-yl)ethyl]-7,8,15,16-tetrahydro-14H-17,20-etheno-9,13-(metheno)-6-oxa-2a,5,15-triazacyclooctadeca[1,2,3-cd]indene-7-carboxylic acid

To a mixture of Example 69L (3.2 mg) in tetrahydrofuran (150 μL) and methanol (150 μL) was added a mixture of lithium hydroxide (1.2 mg) in water (150 μL), and the reaction mixture was allowed to stir overnight. The reaction mixture was quenched with trifluoroacetic acid (8.6 μL) and was purified by reverse-phase HPLC Gilson PLC 2020 using a Luna column (250×30 mm, 10 mm) (5-60% acetonitrile in water containing 0.1% trifluoroacetic acid) to provide the title compound after lyophilyzation. MS (ESI) m/z 916.3 (M+H)⁺.

### Example 73

### (7R,16R,21S)-19-chloro-1-(4-fluorophenyl)-10-{[2-(2-methoxyphenyl)pyrimidin-4-yl]methoxy}-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid

### Example 73A

### (S)-2,3-dihydroxypropyl 4-methylbenzenesulfonate

To a stirring mixture of (S)-(2,2-dimethyl-1,3-dioxolan-4-yl)methyl 4-methylbenzenesulfonate (9 g) in 36 mL of methanol was slowly added 42 mL of 1 M aqueous HCl mixture, and the reaction mixture was stirred at ambient temperature overnight. The mixture was concentrated under reduced pressure to remove most of the methanol. The mixture was carefully poured into 225 mL of saturated aqueous sodium bicarbonate mixture. The mixture was extracted with three portions of ethyl acetate. The combined organic layers were washed with saturated aqueous brine, dried over anhydrous magnesium sulfate, filtered and concentrated onto silica gel. Purification by silica gel flash chromatography on a CombiFlash® Teledyne Isco system using a Teledyne Isco RediSep® Rf gold 330 g silica gel column (eluting with 10-80% of 2:1 ethyl acetate:ethanol in heptane) provided the title compound, which was quickly carried through to the next step. ¹H NMR (400 MHz, dimethyl sulfoxide-d₆) δ ppm 2.42 (s, 3H), 3.18-3.27 (m, 1H), 3.29-3.34 (m, 1H), 3.61 (ttd, 1H), 3.84 (dd, 1H), 3.97-4.05 (m, 1H), 4.68 (t, 1H), 5.10 (d, 1H), 7.48 (d, 2H), 7.73-7.85 (m, 2H). LC/MS (APCI) m/z 247.3 (M+H)⁺.

### Example 73B

### (S)-3-(bis(4-methoxyphenyl)(phenyl)methoxy)-2-hydroxypropyl 4-methylbenzenesulfonate

To a stirring mixture of Example 73A (6.3 g) in 128 mL of dichloromethane at 0° C., was added 4,4′-dimethoxytrityl chloride (9.10 g) in one portion. To the mixture was added N,N-diisopropylethylamine (4.69 mL) dropwise over 15 minutes. The reaction mixture was stirred at 0° C. for an hour and was quenched with saturated aqueous ammonium chloride (100 mL). The layers were separated, and the aqueous layer was extracted with two portions of dichloromethane. The combined organic extracts was dried over anhydrous magnesium sulfate, filtered and concentrated onto silica gel. Purification by flash chromatography on a CombiFlash® Teledyne Isco system using a Teledyne Isco RediSep® Rf gold 330 g silica gel column (eluting 0-50% ethyl acetate/heptane) provided the title compound. ¹H NMR (400 MHz, dimethyl sulfoxide-d₆) δ ppm 2.39 (s, 3H), 2.84 (dd, 1H), 2.94 (dd, 1H), 3.74 (s, 6H), 3.76-3.81 (m, 1H), 3.96 (dd, 1H), 4.02-4.09 (m, 1H), 5.28 (d, 1H), 6.82-6.92 (m, 4H), 7.12-7.18 (m, 4H), 7.19-7.25 (m, 1H), 7.28 (d, 4H), 7.45 (d, 2H), 7.71-7.79 (m, 2H).

### Example 73C

### (R)-3-(bis(4-methoxyphenyl)(phenyl)methoxy)-2-(4-bromo-2-chloro-3-methylphenoxy)propyl 4-methylbenzenesulfonate

A 500 mL round bottom flask, equipped with stir bar and a thermometer, was loaded with Example 73B (10.2 g), Example 64C (4.94 g) and triphenylphosphine (7.31 g). Tetrahydrofuran (186 mL) was added, and di-tert-butyl azodicarboxylate (6.42 g) was added portionwise while keeping the temperature below 25° C. After the addition, the flask was capped, evacuated and backfilled with nitrogen twice. The reaction mixture was placed in a 45° C. pre-heated oil bath, and the mixture was stirred for 90 minutes. After cooling to ambient temperature, the mixture was concentrated onto silica gel. Purification by flash chromatography on a CombiFlash® Teledyne Isco system using a Teledyne Isco RediSep® Rf gold 330 g silica gel column (eluting 5-40% ethyl acetate/heptane) provided a mixture of the product with hydrazine by-product. An additional purification by flash chromatography was performed using the same instrument and column but with a 10-100% dichloromethane/heptane gradient to obtain the title compound. Analytical SFC was performed on an Aurora A5 SFC Fusion and Agilent 1100 system running under Agilent Chemstation software control. The SFC system included a 10-way column switcher, CO₂ pump, modifier pump, oven, and backpressure regulator. The mobile phase comprised of supercritical CO₂ supplied by a beverage-grade CO₂ cylinder with a modifier mixture of methanol at a flow rate of 3 mL/minutes. Oven temperature was at 35° C. and the outlet pressure was at 150 bar. The mobile phase gradient started with 5% modifier held for 0.1 minutes at a flow rate of 1 mL/minutes, then the flow rate was ramped up to 3 mL/minute and held for 0.4 minutes. The modifier was ramped from 5% to 50% over the next 8 minutes at 3 mL/minute then held for 1 minute at 50% modifier (3 mL/minute). The gradient was ramped down from 50% to 5% modifier over 0.5 minute (3 mL/minute). The instrument was fitted with a Whelk-01 (S,S) column with dimensions of 4.6 mm i.d.×150 mm length with 5 μm particles. The minor enantiomer (R) eluted after 7.3 minutes and the major enantiomer (S) eluted after 7.8 minutes. Using this assay the enantiopurity of title compound was determined to be 96% ee (enantiomeric excess). ¹H NMR (400 MHz, dimethyl sulfoxide-d₆) δ ppm 2.33 (s, 3H), 2.41 (s, 3H), 3.16 (d, 2H), 3.69 (d, 6H), 4.19-4.31 (m, 2H), 4.75 (p, 1H), 6.74-6.86 (m, 5H), 7.06-7.12 (m, 4H), 7.13-7.20 (m, 1H), 7.20-7.25 (m, 4H), 7.31-7.37 (m, 2H), 7.39 (d, 1H), 7.61-7.70 (m, 2H).

### Example 73D

### (R)-3-(bis(4-methoxyphenyl)(phenyl)methoxy)-2-(2-chloro-3-methyl-4-(4,4,5,5-tetramethyl-1,3,2-dioxaborolan-2-yl)phenoxy)propyl 4-methylbenzenesulfonate

The title compound was prepared using the conditions described in Example 7H, substituting Example 73C for Example 7G. ¹H NMR (400 MHz, dimethyl sulfoxide-d₆) δ ppm 1.30 (s, 12H), 2.35 (s, 3H), 2.53 (s, 3H), 3.20 (d, 2H), 3.72 (d, 6H), 4.22-4.38 (m, 2H), 4.77-4.90 (m, 1H), 6.74-6.87 (m, 5H), 7.10-7.17 (m, 4H), 7.17-7.30 (m, 5H), 7.32-7.38 (m, 2H), 7.43 (d, 1H), 7.65-7.71 (m, 2H).

### Example 73E

### (R)-ethyl 2-((5-((1S)-4-(((R)-1-(bis(4-methoxyphenyl)(phenyl)methoxy)-3-(tosyloxy)propan-2-yl)oxy)-3-chloro-2-methylphenyl)-6-(4-fluorophenyl)thieno[2,3-d]pyrimidin-4-yl)oxy)-3-(5-((tert-butyldimethylsilyl)oxy)-2-((2-(2-methoxyphenyl)pyrimidin-4-yl)methoxy)phenyl)propanoate

The title compound was prepared using the conditions described in Example 7N, substituting Example 68C for Example 7M, and substituting Example 73D for Example 7H. ¹H NMR (400 MHz, dimethyl sulfoxide-d₆) δ ppm 0.02-0.06 (m, 6H), 0.86 (s, 9H), 0.93 (t, 3H), 1.97 (s, 3H), 2.26-2.32 (m, 1H), 2.35 (s, 3H), 2.40-2.47 (m, 1H), 2.73 (dd, 1H), 3.08-3.26 (m, 2H), 3.64 (d, 6H), 3.73 (s, 3H), 3.86-3.99 (m, 1H), 4.15-4.30 (m, 2H), 4.67-4.78 (m, 1H), 5.04-5.09 (m, 2H), 5.55 (t, 1H), 6.22 (d, 1H), 6.65 (td, 1H), 6.70-6.76 (m, 3H), 6.84-6.95 (m, 2H), 7.01 (td, 1H), 7.08-7.32 (m, 11H), 7.31-7.41 (m, 4H), 7.41-7.60 (m, 2H), 7.63-7.70 (m, 2H), 8.60 (s, 1H), 8.80 (d, 1H).

### Example 73F

### (R)-ethyl 2-((5-((1S)-4-(((R)-1-(bis(4-methoxyphenyl)(phenyl)methoxy)-3-(tosyloxy)propan-2-yl)oxy)-3-chloro-2-methylphenyl)-6-(4-fluorophenyl)thieno[2,3-d]pyrimidin-4-yl)oxy)-3-(5-hydroxy-2-((2-(2-methoxyphenyl)pyrimidin-4-yl)methoxy)phenyl)propanoate

Example 73E (1.76 g) was dissolved in dichloromethane (61.2 mL) and was treated with tetrabutylammonium fluoride (1.224 mL, 1 M in tetrahydrofuran) at ambient temperature for 15 minutes. The mixture was concentrated onto silica gel and purification by flash chromatography on a CombiFlash® Teledyne Isco system using a Teledyne Isco RediSep® Rf gold 80 g silica gel column (eluting with 10-100% ethyl acetate/heptane) provided the title compound. ¹H NMR (400 MHz, dimethyl sulfoxide-d₆) δ ppm 1.00 (t, 3H), 1.93 (s, 3H), 2.35 (s, 3H), 2.71 (dd, 1H), 3.09 (dd, 1H), 3.24 (dd, 1H), 3.65 (d, 6H), 3.73 (s, 3H), 3.95-4.07 (m, 2H), 4.19-4.35 (m, 2H), 4.72-4.86 (m, 1H), 4.97-5.09 (m, 2H), 5.40 (dd, 1H), 5.93 (d, 1H), 6.56 (dd, 1H), 6.69-6.77 (m, 4H), 6.78-6.85 (m, 2H), 6.88-6.95 (m, 1H), 7.01 (td, 1H), 7.05-7.28 (m, 12H), 7.31-7.40 (m, 4H), 7.41-7.47 (m, 2H), 7.50 (dd, 1H), 7.66-7.75 (m, 2H), 8.59 (s, 1H), 8.81 (s, 1H), 8.83 (d, 1H).

### Example 73G

### ethyl (7R,16S,21S)-16-{[bis(4-methoxyphenyl)(phenyl)methoxy]methyl}-19-chloro-1-(4-fluorophenyl)-10-{[2-(2-methoxyphenyl)pyrimidin-4-yl]methoxy}-20-methyl-7,8,15,16-tetrahydro-18,21-etheno-13,9-(metheno)-6,14,17-trioxa-2-thia-3,5-diazacyclononadeca[1,2,3-cd]indene-7-carboxylate

To a mixture of Example 73F (535 mg) in N,N-dimethylformamide (53.9 mL) was added cesium carbonate (1317 mg). The reaction mixture was stirred at 40° C. for 2 hours. The mixture was cooled to ambient temperature, poured into a separatory funnel, and diluted with ethyl acetate and water. The layers were separated, and the aqueous layer was extracted with two portions of ethyl acetate. The combined organics were washed with brine, dried over anhydrous magnesium sulfate, filtered and concentrated onto silica gel. Purification by silica gel chromatography on a CombiFlash® Teledyne Isco system using a Teledyne Isco RediSep® Rf gold 40 g silica gel column (eluting with 20-100% ethyl acetate/heptane) provided the title compound. LC/MS (APCI) m/z 1151.1 (M+H)⁺.

### Example 73H

### ethyl (7R,16R,21S)-19-chloro-1-(4-fluorophenyl)-16-(hydroxymethyl)-10-{[2-(2-methoxyphenyl)pyrimidin-4-yl]methoxy}-20-methyl-7,8,15,16-tetrahydro-18,21-etheno-13,9-(metheno)-6,14,17-trioxa-2-thia-3,5-diazacyclononadeca[1,2,3-cd]indene-7-carboxylate

Example 73G (350 mg) was treated with a mixture of methanol (1.5 mL), dichloromethane (1.5 mL) and formic acid (1.5 mL) for 15 minutes. The mixture was then carefully poured into 50 mL of saturated aqueous sodium bicarbonate mixture and was extracted with three portions of ethyl acetate. The combined organic layers were washed with saturated aqueous brine, dried over anhydrous magnesium sulfate, filtered and concentrated onto silica gel. Purification by silica chromatography on a CombiFlash® Teledyne Isco system using a Teledyne Isco RediSep® Rf gold 24 g silica gel column (eluting with 20-100% ethyl acetate/heptane) provided the title compound. LC/MS (APCI) m/z 849.3 (M+H)⁺.

### Example 73I

### ethyl (7R,16S,21S)-19-chloro-1-(4-fluorophenyl)-10-{[2-(2-methoxyphenyl)pyrimidin-4-yl]methoxy}-20-methyl-16-{[(4-methylbenzene-1-sulfonyl)oxy]methyl}-7,8,15,16-tetrahydro-18,21-etheno-13,9-(metheno)-6,14,17-trioxa-2-thia-3,5-diazacyclononadeca[1,2,3-cd]indene-7-carboxylate

To a mixture of Example 73H (183 mg) and triethylamine (90 μL) in dichloromethane (2.2 mL) was added para-toluenesulfonyl chloride (82 mg) in one portion. The mixture was stirred at ambient temperature overnight. The mixture was concentrated onto silica gel and purification by flash chromatography on a CombiFlash® Teledyne Isco system using a Teledyne Isco RediSep® Rf gold 24 g silica gel column (eluting with 20-100% ethyl acetate/heptane) provided the title compound. LC/MS (APCI) m/z 1003.1 (M+H)⁺.

### Example 73J

### ethyl (7R,16R,21S)-19-chloro-1-(4-fluorophenyl)-10-{[2-(2-methoxyphenyl)pyrimidin-4-yl]methoxy}-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

A 20 mL vial was charged with Example 73I (670 mg), 1-methylpiperazine (2.0 g) and N,N-dimethylformamide (2.2 mL). The vial was capped and stirred at 45° C. for 24 hours. The mixture was poured into 30 mL of water, and the precipitate obtained was sonicated for a few minutes. The material was filtered and washed with 50 mL of water. The material was collected and dried under high vacuum to obtain the title compound. LC/MS (APCI) m/z 931.1 (M+H)⁺.

### Example 73K

### (7R,16R,21S)-19-chloro-1-(4-fluorophenyl)-10-{[2-(2-methoxyphenyl)pyrimidin-4-yl]methoxy}-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid

Example 73J (560 mg) was dissolved in methanol (8 mL) and tetrahydrofuran (16 mL), and the mixture was cooled to 0° C. To the resulting stirred mixture was slowly added 1 molar aqueous lithium hydroxide (12 mL), and the reaction mixture was stirred at ambient temperature overnight. The mixture was concentrated to remove the volatiles, and the aqueous mixture was treated with acetic acid until the pH was slightly acidic. The precipitate that formed was dissolved by the addition of 5 mL of acetonitrile. The mixture was purified by reverse phase prep LC using a Gilson 2020 system (Luna, C-18, 250×50 mm column, mobile phase A: 0.1% trifluoroacetic acid in water; B: acetonitrile; 5-75% B to A gradient at 70 mL/minute) to obtain the title compound. ¹H NMR (400 MHz, dimethyl sulfoxide-d₆) δ ppm 2.23 (s, 3H), 2.70-2.77 (m, 2H), 2.79 (s, 3H), 2.83-2.95 (m, 1H), 2.95-3.24 (m, 4H), 3.28-3.47 (m, 4H), 3.77 (s, 3H), 3.87 (dd, 1H), 4.36 (dd, 1H), 4.47 (d, 1H), 4.59 (q, 1H), 5.18 (q, 2H), 5.67 (d, 1H), 6.16 (dd, 1H), 6.84 (dd, 1H), 6.88-6.93 (m, 1H), 6.97 (d, 1H), 7.06 (t, 1H), 7.13-7.24 (m, 6H), 7.47 (td, 1H), 7.51-7.58 (m, 2H), 8.75 (s, 1H), 8.89 (d, 1H). MS (ESI) m/z 903.2 (M+H)⁺.

### Example 74

### (7R,20S)-18-chloro-1-(4-fluorophenyl)-10-{[2-(2-methoxyphenyl)pyrimidin-4-yl]methoxy}-19-methyl-15-[3-(4-methylpiperazin-1-yl)propanoyl]-7,8,15,16-tetrahydro-14H-17,20-etheno-13,9-(metheno)-6-oxa-2-thia-3,5,15-triazacyclooctadeca[1,2,3-cd]indene-7-carboxylic acid

### Example 74A

### (R)-ethyl 2-((5-bromo-6-(4-fluorophenyl)thieno[2,3-d]pyrimidin-4-yl)oxy)-3-(5-(hydroxymethyl)-2-((2-(2-methoxyphenyl)pyrimidin-4-yl)methoxy)phenyl)propanoate

The title compound was prepared as described in Example 61J, substituting Example 61I with Example 61H. MS (ESI) m/z 747.1 (M+H)⁺.

### Example 74B

### (R)-ethyl 2-((5-bromo-6-(4-fluorophenyl)thieno[2,3-d]pyrimidin-4-yl)oxy)-3-(5-((N-(tert-butoxycarbonyl)-2-(trimethylsilyl)ethylsulfonamido)methyl)-2-((2-(2-methoxyphenyl)pyrimidin-4-yl)methoxy)phenyl)propanoate

To a cold (ice bath) tetrahydrofuran (2 mL) mixture of Example 74A (0.257 g), tert-butyl (2-(trimethylsilyl)ethyl)sulfonylcarbamate (0.12 g) and triphenylphosphine (0.15 g) was added a tetrahydrofuran mixture of (E)-di-tert-butyl diazene-1,2-dicarboxylate (0.12 g, 1 mL) dropwise by syringe. The mixture was stirred at room temperature for 2 hours. The mixture was concentrated under reduced pressure, and the residue was dissolved in ethyl acetate (20 mL). The ethyl acetate mixture was washed successively with water and brine, dried with anhydrous sodium sulfate, and filtered. The solvents were removed under reduced pressure, and the reaction mixture was purified by silica gel chromatography using a Teledyne ISCO CombiFlash® system and RediSep® Rf SF40-80 g column, eluting with 0-10% ethyl acetate/heptane, to provide the title compound. MS (ESI) m/z 1010.0 (M+H)⁺.

### Example 74C

### (2R)-ethyl 3-(5-((N-(tert-butoxycarbonyl)-2-(trimethylsilyl)ethylsulfonamido)methyl)-2-((2-(2-methoxyphenyl)pyrimidin-4-yl)methoxy)phenyl)-2-((5-((1S)-3-chloro-4-(((tert-butyldimethylsilyl)oxy)methyl)-2-methylphenyl)-6-(4-fluorophenyl)thieno[2,3-d]pyrimidin-4-yl)oxy)propanoate

The title compound was prepared as described in Example 61I, substituting Example 61H with Example 74B. MS (APCI) m/z 1084.2 (M+H)⁺.

### Example 74D

### (2R)-ethyl 2-((5-((1S)-3-chloro-4-(hydroxymethyl)-2-methylphenyl)-6-(4-fluorophenyl)thieno[2,3-d]pyrimidin-4-yl)oxy)-3-(2-((2-(2-methoxyphenyl)pyrimidin-4-yl)methoxy)-5-((2-(trimethylsilyl)ethylsulfonamido)methyl)phenyl)propanoate

To a mixture of Example 74C (0.124 g) in dichloromethane (1 mL) was added trifluoroacetic acid (1 mL). The mixture was stirred at room temperature for 24 hours. The solvents were removed under reduced pressure, and the residue was treated with dichloromethane/water (10:1, 5 mL). Solid sodium bicarbonate (100 mg) was added, and the mixture was stirred at room temperature for 3 hours. Dichloromethane (10 mL) and water (5 mL) were added, and the mixture was filtered through a Biotage® Isolute Phase Separator column. The dichloromethane mixture was concentrated. The residue was purified by silica gel chromatography using a Teledyne ISCO CombiFlash® system and RediSep® Rf SF25-40 g column, eluting with 1-10% methanol in dichloromethane, to provide the title compound. MS (ESI) m/z 984.3 (M+H)⁺.

### Example 74E

### ethyl (7R,20S)-18-chloro-1-(4-fluorophenyl)-10-{[2-(2-methoxyphenyl)pyrimidin-4-yl]methoxy}-19-methyl-15-[2-(trimethylsilyl)ethanesulfonyl]-7,8,15,16-tetrahydro-14H-17,20-etheno-13,9-(metheno)-6-oxa-2-thia-3,5,15-triazacyclooctadeca[1,2,3-cd]indene-7-carboxylate

The title compound was prepared as described in Example 61K, substituting Example 61J with Example 74D. MS (ESI) m/z 966.3 (M+H)⁺.

### Example 74F

### ethyl (7R,20S)-18-chloro-1-(4-fluorophenyl)-10-{[2-(2-methoxyphenyl)pyrimidin-4-yl]methoxy}-19-methyl-7,8,15,16-tetrahydro-14H-17,20-etheno-13,9-(metheno)-6-oxa-2-thia-3,5,15-triazacyclooctadeca[1,2,3-cd]indene-7-carboxylate

The title compound was prepared as described in Example 61J, substituting Example 61I with Example 74E. MS (ESI) m/z 802.2 (M+H)⁺.

### Example 74G

### ethyl (7R,20S)-18-chloro-1-(4-fluorophenyl)-10-{[2-(2-methoxyphenyl)pyrimidin-4-yl]methoxy}-19-methyl-15-[3-(4-methylpiperazin-1-yl)propanoyl]-7,8,15,16-tetrahydro-14H-17,20-etheno-13,9-(metheno)-6-oxa-2-thia-3,5,15-triazacyclooctadeca[1,2,3-cd]indene-7-carboxylate

To a mixture of 1-[bis(dimethylamino)methylene]-1H-1,2,3-triazolo[4,5-b]pyridinium 3-oxide hexafluorophosphate (6 mg) in N,N-dimethylformamide (0.5 mL) was added 3-(4-methylpiperazin-1-yl)propanoic acid (5 mg). The mixture was stirred at room temperature for 5 minutes. Example 74F (10 mg) was added, and the mixture was stirred at room temperature for 3 hours. The reaction mixture was diluted with N,N-dimethylformamide/water (1:1, 1 mL) and was purified by reverse phase HPLC using a Gilson system (Luna column, 250×30 mm, flow rate 50 mL/min) using a gradient of 20-100% acetonitrile in water containing 0.1% v/v trifluoroacetic acid over 30 minutes. The desired product containing fractions were lyophilized to provide title compound. MS (ESI) m/z 956.4 (M+H)⁺.

### Example 74H

### (7R,20S)-18-chloro-1-(4-fluorophenyl)-10-{[2-(2-methoxyphenyl)pyrimidin-4-yl]methoxy}-19-methyl-15-[3-(4-methylpiperazin-1-yl)propanoyl]-7,8,15,16-tetrahydro-14H-17,20-etheno-13,9-(metheno)-6-oxa-2-thia-3,5,15-triazacyclooctadeca[1,2,3-cd]indene-7-carboxylic acid

The title compound was prepared as described in Example 1W, substituting Example 1V with Example 74G. ¹H NMR (400 MHz, dimethyl sulfoxide-d₆) δ ppm 8.61 (m, 2H), 7.45 (m, 2H), 7.26 (m, 5H), 7.08 (m, 5H), 6.79 (d, 1H), 6.21 (s, 1H), 5.85 (m, 1H), 5.12 (m, 3H), 4.67 (m, 1H), 4.43 (m, 1H), 3.72 (s, 3H), 2.67 (m, 4H), 1.62 (s, 3H). MS (ESI) m/z 928.3 (M+H)⁺.

### Example 75

### (7R,16R,21R)-2,19-dichloro-1-(4-fluorophenyl)-10-{[2-(2-methoxyphenyl)pyrimidin-4-yl]methoxy}-20-methyl-16-[(4-methylpiperazin-1-yl)methyl]-7,8,15,16-tetrahydro-18,21-etheno-9,13-(metheno)-6,14,17-trioxa-2a,5-diazacyclononadeca[1,2,3-cd]indene-7-carboxylic acid

### Example 75A

### (R)-ethyl 3-(5-((tert-butyldimethylsilyl)oxy)-2-((2-(2-methoxyphenyl)pyrimidin-4-yl)methoxy)phenyl)-2-((6-chloro-7-(4-fluorophenyl)-8-iodopyrrolo[1,2-a]pyrazin-1-yl)oxy)propanoate

A mixture of Example 68B (152 mg), Example 69G (116 mg) and cesium carbonate (276 mg) in tert-butanol (5.6 mL) was warmed at 27° C. for 24 hours. The reaction mixture was diluted with water and brine and extracted with ethyl acetate three times. The combined organic layers were dried over sodium sulfate, filtered and concentrated. The crude residue was purified by normal phase MPLC on a Teledyne Isco Combiflash Rf+ (5-70% ethyl acetate in heptanes) to provide the title compound. MS (ESI) m/z 909.0 (M+H)⁺.

### Example 75B

### (2R)-ethyl 2-((8-((1R)-4-(((R)-1-(bis(4-methoxyphenyl)(phenyl)methoxy)-3-(4-methylpiperazin-1-yl)propan-2-yl)oxy)-3-chloro-2-methylphenyl)-6-chloro-7-(4-fluorophenyl)pyrrolo[1,2-a]pyrazin-1-yl)oxy)-3-(5-((tert-butyldimethylsilyl)oxy)-2-((2-(2-methoxyphenyl)pyrimidin-4-yl)methoxy)phenyl)propanoate

A mixture of Example 64K (110 mg), Example 75A (106 mg), bis(di-tert-butyl(4-dimethylaminophenyl)phosphine)dichloropalladium(II) (8.3 mg) and cesium carbonate (114 mg) in degassed tetrahydrofuran (1.2 mL) and water (290 μL) was stirred for 46 hours. 1-Pyrrolidinecarboxylic acid ammonium salt (1.9 mg) was added and the reaction mixture was stirred for 30 minutes. The reaction mixture was filtered over diatomaceous earth, washing with ethyl acetate. The mixture was diluted with brine and extracted with ethyl acetate three times. The combined organic layers were dried over sodium sulfate, filtered and concentrated under reduced pressure. The crude residue was purified by normal phase MPLC on a Teledyne Isco Combiflash Rf+ (0-6.5% methanol in dichloromethane) to provide the title compound. MS (ESI) m/z 1382.3 (M+H)⁺.

### Example 75C

### (R)-ethyl 2-(((R)-6-chloro-8-((1R)-3-chloro-4-(((R)-1-hydroxy-3-(4-methylpiperazin-1-yl)propan-2-yl)oxy)-2-methylphenyl)-7-(4-fluorophenyl)pyrrolo[1,2-a]pyrazin-1-yl)oxy)-3-(5-hydroxy-2-((2-(2-methoxyphenyl)pyrimidin-4-yl)methoxy)phenyl)propanoate

To a mixture of Example 75B (23 mg) in dichloromethane (100 μL) and methanol (100 μL) was added formic acid (96 μL), and the reaction mixture was stirred for 90 minutes. The reaction mixture was quenched slowly with saturated sodium bicarbonate mixture and was extracted with ethyl acetate three times. The combined organic layers were dried over sodium sulfate, filtered and concentrated under reduced pressure to give a crude residue that was used without further purification. To the residue in tetrahydrofuran (300 μL) was added tetrabutyl ammonium fluoride (1 M in tetrahydrofuran, 50 μL), and the reaction mixture was allowed to stir for 45 minutes. The reaction mixture was quenched with saturated ammonium chloride mixture and was extracted with ethyl acetate three times. The crude residue was purified by reverse-phase HPLC on a Gilson PLC 2020 using a Luna column (250×50 mm, 10 m, 5-80% acetonitrile in water containing 0.1% trifluoroacetic acid) to provide the title compound. MS (ESI) m/z 967.1 (M+H)⁺.

### Example 75D

### (7R,16R,21R)-2,19-dichloro-1-(4-fluorophenyl)-10-{[2-(2-methoxyphenyl)pyrimidin-4-yl]methoxy}-20-methyl-16-[(4-methylpiperazin-1-yl)methyl]-7,8,15,16-tetrahydro-18,21-etheno-9,13-(metheno)-6,14,17-trioxa-2a,5-diazacyclononadeca[1,2,3-cd]indene-7-carboxylic acid

A mixture of Example 75C (20.6 mg) triphenylphosphine (11.2 mg) and N,N,N,N-tetramethylazodicarboxamide (7.3 mg) was heated at 50° C. overnight. More triphenylphosphine (11 mg) and N,N,N,N-tetramethylazodicarboxamide (7.3 mg) was added and heating was continued overnight. Additional triphenylphosphine (11 mg) and N,N,N,N-tetramethylazodicarboxamide (7.3 mg) were added and heating was continued for 4 hours. Additional triphenylphosphine (11 mg) and N,N,N,N-tetramethylazodicarboxamide (7.3 mg) was added and heating was continued for 2 days. The reaction mixture was cooled, diluted with ethyl acetate, filtered over diatomaceous earth and concentrated to give a crude material. To a mixture of the crude material in tetrahydrofuran (240 μL) and methanol (240 μL) was added lithium hydroxide (7.7 mg) in water (240 μL), and the reaction mixture was stirred overnight. The reaction mixture was quenched with trifluoroacetic acid (33 μL) and was purified by reverse-phase HPLC on a Gilson PLC 2020 using a Luna column (250×30 mm, 10 m) (5-70% acetonitrile in water containing 0.1% trifluoroacetic acid) to provide the title compound. ¹H NMR (400 MHz, dimethyl sulfoxide-d₆) δ ppm 8.87 (d, 1H), 7.99 (d, 1H), 7.57-7.50 (m, 2H), 7.49-7.43 (m, 1H), 7.34 (d, 1H), 7.21-7.11 (m, 6H), 7.09-7.03 (m, 2H), 6.93-6.85 (m, 2H), 6.78 (dd, 1H), 6.11-6.05 (m, 1H), 5.75 (d, 1H), 5.16 (dd, 2H), 4.66-4.57 (m, 1H), 4.44 (d, 1H), 4.31 (dd, 1H), 3.85-3.72 (m, 4H), 3.15-2.85 (m, 6H), 2.78 (s, 3H), 3.75-2.67 (m, 2H), 2.14 (s, 3H). MS (ESI) m/z 921.3 (M+H)⁺.

### Example 76

### (7R,20S)-18-chloro-1-(4-fluorophenyl)-19-methyl-10-[(4-{3-[2-(4-methylpiperazin-1-yl)ethoxy]phenyl}pyrimidin-2-yl)methoxy]-7,8-dihydro-14H,16H-17,20-etheno-13,9-(metheno)-6,15-dioxa-2-thia-3,5-diazacyclooctadeca[1,2,3-cd]indene-7-carboxylic acid

### Example 76A

### (R)-ethyl 2-acetoxy-3-(5-(((tert-butyldimethylsilyl)oxy)methyl)-2-((2-(methylthio)pyrimidin-4-yl)methoxy)phenyl)propanoate

To a mixture of Example 61E (2.5 g), (2-(methylthio)pyrimidin-4-yl)methanol (1.54 g) and triphenylphosphine (3.3 g) in toluene (50 mL) was added N,N,N,N′-tetramethylazodicarboxamide (1.3 g). The reaction mixture was stirred at room temperature overnight. The material was removed by filtration. The filtrate was concentrated, and the residue was purified by silica gel chromatography with 30% ethyl acetate in heptane to provide the title compound. MS (ESI) m/z 535 (M+NH₄)⁺.

### Example 76B

### (R)-ethyl 3-(5-(((tert-butyldimethylsilyl)oxy)methyl)-2-((2-(methylthio)pyrimidin-4-yl)methoxy)phenyl)-2-hydroxypropanoate

To a mixture of Example 76A (2.7 g) in ethanol (50 mL) was added sodium ethoxide (1.7 g, 20% in ethanol). The mixture was stirred at room temperature for 30 minutes. The reaction mixture was quenched with water (100 mL) and was extracted with ethyl acetate (200 mL×2). The organic phase was concentrated and was purified by silica gel chromatography, eluting with 40% ethyl acetate in hexane to provide the title compound. MS (ESI) m/z 493 (M+NH₄)⁺.

### Example 76C

### (R)-ethyl 2-((5-bromo-6-(4-fluorophenyl)thieno[2,3-d]pyrimidin-4-yl)oxy)-3-(5-(((tert-butyldimethylsilyl)oxy)methyl)-2-((2-(methylthio)pyrimidin-4-yl)methoxy)phenyl)propanoate

To a mixture of Example 1D (0.9 g) and Example 76B (0.9 g) in dichloromethane (5 mL) was added tert-butanol (10 mL) and cesium carbonate (0.7 g) and the mixture was stirred at 65° C. overnight. The reaction mixture was partitioned between ethyl acetate (100 mL) and water (100 mL). The organic phase was concentrated, and the residue was purified by silica gel chromatography, eluting with 10% methanol in ethyl acetate to provide the title compound. MS (ESI) m/z 800 (M+NH₄)⁺.

### Example 76D

### (2R)-ethyl 3-(5-(((tert-butyldimethylsilyl)oxy)methyl)-2-((2-(methylthio)pyrimidin-4-yl)methoxy)phenyl)-2-((5-((1S)-4-(((tert-butyldimethylsilyl)oxy)methyl)-3-chloro-2-methylphenyl)-6-(4-fluorophenyl)thieno[2,3-d]pyrimidin-4-yl)oxy)propanoate

A mixture of Example 76C (1.4 g), tert-butyl((2-chloro-3-methyl-4-(4,4,5,5-tetramethyl-1,3,2-dioxaborolan-2-yl)benzyl)oxy)dimethylsilane (0.77 g), bis(di-tert-butyl(4-dimethylaminophenyl)phosphine)dichloropalladium(II) (124 mg) and K₃PO₄ (0.9 g) was evacuated and filled with nitrogen gas. To the mixture were added degassed tetrahydrofuran (50 mL) and water (12 mL). The reaction mixture was stirred at 40° C. overnight. The reaction mixture was quenched with water (100 mL) and was extracted with ethyl acetate (2×100 mL). The organic phase was concentrated, and the residue was purified by silica gel chromatography, eluting with 30% ethyl acetate in heptane to provide the title compound. MS (ESI) m/z 990 (M+NH₄)⁺.

### Example 76E

### (R)-ethyl 2-(((S)-5-((1S)-3-chloro-4-(hydroxymethyl)-2-methylphenyl)-6-(4-fluorophenyl)thieno[2,3-d]pyrimidin-4-yl)oxy)-3-(5-(hydroxymethyl)-2-((2-(methylthio)pyrimidin-4-yl)methoxy)phenyl)propanoate

A mixture of Example 76D (1.3 g) in tetrahydrofuran (20 mL) was cooled to 0° C., and tetrabutylammonium fluoride (1.5 mL, 1M in tetrahydrofuran) was added. The mixture was stirred at room temperature for 3 hours. The reaction mixture was quenched with water (100 mL) and was extracted with ethyl acetate (2×100 mL). The organic phase was concentrated, and the residue was purified by silica gel chromatography, eluting with 80% ethyl acetate in heptane to provide the title compound. MS (ESI) m/z 762 (M+NH₄)⁺.

### Example 76F

### ethyl (7R,20S)-18-chloro-1-(4-fluorophenyl)-19-methyl-10-{[2-(methylsulfanyl)pyrimidin-4-yl]methoxy}-7,8-dihydro-14H,16H-17,20-etheno-13,9-(metheno)-6,15-dioxa-2-thia-3,5-diazacyclooctadeca[1,2,3-cd]indene-7-carboxylate

A mixture of N,N,N′,N′-tetramethylazodicarboxamide (580 mg) in toluene (6 mL) was evacuated, filled with nitrogen, and cooled to 0° C. To this mixture was added tributylphosphine (465 mg). The mixture was warmed up to room temperature and was stirred at room temperature for 10 minutes. A mixture of Example 76E (350 mg) in toluene (1 mL) was added into the reaction and the mixture was stirred overnight. The reaction mixture was quenched with water (100 mL) and was extracted with ethyl acetate (2×100 mL). The organic phase was concentrated, and the residue was purified by silica gel chromatography, eluting with 80% ethyl acetate in heptane to provide the title compound. MS (ESI) m/z 744 (M+NH₄)⁺.

### Example 76G

### (7R,20S)-18-chloro-1-(4-fluorophenyl)-19-methyl-10-[(4-{3-[2-(4-methylpiperazin-1-yl)ethoxy]phenyl}pyrimidin-2-yl)methoxy]-7,8-dihydro-14H,16H-17,20-etheno-13,9-(metheno)-6,15-dioxa-2-thia-3,5-diazacyclooctadeca[1,2,3-cd]indene-7-carboxylic acid

A mixture of Example 76F (30 mg), (3-(2-(4-methylpiperazin-1-yl)ethoxy)phenyl)boronic acid (21 mg), tetrakis(triphenylphosphine)palladium(0) (9 mg) and copper(I) thiophene-2-carboxylate (31 mg) in anhydrous tetrahydrofuran (1 mL) in a sealed microwave tube was degassed and filled with argon. The reaction mixture was processed in a Biotage® Initiator microwave reactor at 90° C. for 30 minutes. The reaction mixture was directly loaded onto a silica gel column and was eluted with 30-80% ethyl acetate/heptane, to provide an intermediate that was dissolved in a mixed solvent of tetrahydrofuran (2 mL), methanol (1 mL) and water (1 mL). LiOH monohydrate (30 mg) was added and the mixture was stirred overnight. Trifluoroacetic acid (1 mL) was added to the reaction. The reaction mixture was purified by reverse phase HPLC using a Gilson system and a gradient of 30% to 100% acetonitrile water with 0.1% trifluoroacetic acid. The desired product containing fractions were lyophilized to provide the title compound. ¹H NMR (501 MHz, methanol-d₄) δ ppm 8.60 (d, 1H), 8.43 (s, 1H), 8.02-7.89 (m, 2H), 7.45-7.32 (m, 2H), 7.34-7.28 (m, 2H), 7.19-7.05 (m, 4H), 7.02-6.87 (m, 2H), 6.74 (d, 1H), 6.66 (d, 1H), 6.01 (dd, 1H), 5.16 (d, 1H), 5.10-4.92 (m, 2H), 4.29 (td, 2H), 3.42-3.31 (m, 2H), 3.30 (p, 8H), 3.17-2.96 (m, 7H), 2.87 (s, 2H), 1.60 (s, 3H). MS (ESI) m/z 888 (M+H)⁺.

### Example 77

### (7R,20S)-18-chloro-1-(4-fluorophenyl)-10-{[2-(3-methoxyphenyl)pyrimidin-4-yl]methoxy}-19-methyl-7,8-dihydro-14H,16H-17,20-etheno-13,9-(metheno)-6,15-dioxa-2-thia-3,5-diazacyclooctadeca[1,2,3-cd]indene-7-carboxylic acid

### Example 77A

### (R)-ethyl 2-acetoxy-3-(5-(((tert-butyldimethylsilyl)oxy)methyl)-2-((2-(methylthio)pyrimidin-4-yl)methoxy)phenyl)propanoate

To a mixture of Example 61E (2.5 g), Example 7B (0.985 g) and triphenylphosphine (3.3 g) in toluene (50 mL) was added tetramethylazodicarboxamide (1.3 g). The reaction mixture was stirred at room temperature overnight. The material was removed by filtration. The filtrate was concentrated and was purified by flash chromatography with 30% ethyl acetate in heptane to give the title compound. MS (ESI) m/z 535 (M+H)⁺.

### Example 77B

### (R)-ethyl 3-(5-(((tert-butyldimethylsilyl)oxy)methyl)-2-((2-(methylthio)pyrimidin-4-yl)methoxy)phenyl)-2-hydroxypropanoate

To a mixture of Example 77A (2.7 g) in ethanol (50 mL) was added sodium ethoxide (1.7 g, 20% in ethanol). The mixture was stirred at room temperature for 30 minutes. The reaction mixture was quenched with water (100 mL) and was extracted with ethyl acetate (200×2). The organic phase was concentrated and was purified by flash chromatography with 40% ethyl acetate in hexane to provide the title compound. MS (ESI) m/z 493 (M+H)⁺.

### Example 77C

### (R)-ethyl 2-((5-bromo-6-(4-fluorophenyl)thieno[2,3-d]pyrimidin-4-yl)oxy)-3-(5-(((tert-butyldimethylsilyl)oxy)methyl)-2-((2-(methylthio)pyrimidin-4-yl)methoxy)phenyl)propanoate

To a mixture of Example 77B (0.9 g) in dichloromethane (5 mL) was added Example 1D (0.9 g). To the resulting mixture was added tert-butanol (10 mL) and Cs₂CO₃ (0.7 g) and the reaction mixture was stirred at 65° C. overnight. The reaction mixture was partitioned between ethyl acetate (100 mL) and water (100 mL). The organic phase was concentrated and was purified by flash chromatography with 10% methanol in ethyl acetate to provide the title compound. MS (ESI) m/z 800 (M+H)⁺.

### Example 77D

### (2R)-ethyl 3-(5-(((tert-butyldimethylsilyl)oxy)methyl)-2-((2-(methylthio)pyrimidin-4-yl)methoxy)phenyl)-2-((5-((1S)-4-(((tert-butyldimethylsilyl)oxy)methyl)-3-chloro-2-methylphenyl)-6-(4-fluorophenyl)thieno[2,3-d]pyrimidin-4-yl)oxy)propanoate

A flask containing Example 77C (430 mg), Example 20G (320 mg), bis(di-tert-butyl(4-dimethylaminophenyl)phosphine)dichloropalladium(II) (38 mg) and K₃PO₄ (285 mg) was degassed and filled with argon. To this mixture a degassed and argon-sparged mixture of tetrahydrofuran (12 mL) and water (3 mL) was added, and the reaction mixture was stirred at 40° C. overnight. The reaction mixture was concentrated, diluted in dichloromethane (2 mL), and purified by flash chromatography (30% ethyl acetate in heptane) to provide the title compound. MS (ESI) m/z 990 (M+H)⁺.

### Example 77E

### (2R)-ethyl 2-((5-((1S)-3-chloro-4-(hydroxymethyl)-2-methylphenyl)-6-(4-fluorophenyl)thieno[2,3-d]pyrimidin-4-yl)oxy)-3-(5-(hydroxymethyl)-2-((2-(methylthio)pyrimidin-4-yl)methoxy)phenyl)propanoate

To a mixture of Example 77D (700 mg) in tetrahydrofuran (10 mL) cooled in an ice bath was added tetrabutyl ammonium fluoride (1.4 mL, IM in tetrahydrofuran). The reaction mixture was stirred at 0° C. for 1 hour. The reaction mixture was partitioned between water (100 mL) and ethyl acetate (200 mL). The organic phase was concentrated and was purified by flash chromatography (50% ethyl acetate in heptane) to provide the title compound. MS (ESI) m/z 762 (M+H)⁺.

### Example 77F

### ethyl (7R,20S)-18-chloro-1-(4-fluorophenyl)-19-methyl-10-{[2-(methylsulfanyl)pyrimidin-4-yl]methoxy}-7,8-dihydro-14H,16H-17,20-etheno-13,9-(metheno)-6,15-dioxa-2-thia-3,5-diazacyclooctadeca[1,2,3-cd]indene-7-carboxylate

A mixture of Example 77E (270 mg) in toluene (10 mL) was heated to 70° C. overnight. After cooling to room temperature, the reaction mixture was loaded onto a silica gel column and was purified by flash chromatography (30% ethyl acetate in heptane) to provide the title compound. MS (ESI) m/z 744 (M+H)⁺.

### Example 77G

### (7R,20S)-18-chloro-1-(4-fluorophenyl)-10-{[2-(3-methoxyphenyl)pyrimidin-4-yl]methoxy}-19-methyl-7,8-dihydro-14H,16H-17,20-etheno-13,9-(metheno)-6,15-dioxa-2-thia-3,5-diazacyclooctadeca[1,2,3-cd]indene-7-carboxylic acid

A mixture of Example 77F (40 mg), (3-methoxyphenyl)boronic acid (16 mg), tetrakis(triphenylphosphine)palladium(0) (12 mg) and copper(I)-thiophene-2-carboxylate (41 mg) in tetrahydrofuran (1 mL) in a sealed microwave tube was degassed and filled with argon. The reaction mixture was processed in a Biotage® Initiator microwave reactor at T=90° C. for 30 minutes. The reaction mixture was purified by flash chromatography (50% ethyl acetate in heptane) to give an intermediate which was dissolved in a mixed solvent of tetrahydrofuran (2 mL), methanol (1 mL) and water (1 mL). LiOH (30 mg) was added and the mixture was stirred overnight. Trifluoroacetic acid (1 mL) was added to the reaction and the mixture was concentrated. The residue was purified by HPLC (Zorbax, C-18, 250×4.6 mm column, Mobile phase A: 0.1% trifluoroacetic acid in H₂O; B: 0.1% trifluoroacetic acid in CH₃CN; 0-70% gradient) to provide the title compound. ¹H NMR (501 MHz, methanol-d₄) δ ppm 8.66 (d, J=5.4 Hz, 1H), 8.49 (s, 1H), 7.74 (dd, J=7.6, 1.8 Hz, 1H), 7.54 (ddd, J=8.6, 7.4, 1.8 Hz, 1H), 7.49 (d, J=5.4 Hz, 1H), 7.39 (d, J=7.8 Hz, 1H), 7.32 (d, J=7.8 Hz, 1H), 7.21-7.14 (m, 3H), 7.14-7.07 (m, 2H), 6.99-6.93 (m, 2H), 6.74 (d, J=8.4 Hz, 1H), 6.64 (d, J=2.2 Hz, 1H), 6.02 (dd, J=10.4, 3.9 Hz, 1H), 5.20 (d, J=15.3 Hz, 1H), 5.08 (d, J=15.3 Hz, 1H), 5.01 (d, J=12.8 Hz, 1H), 4.68-4.60 (m, 3H), 3.88 (s, 3H), 3.39 (dd, J=15.0, 3.9 Hz, 1H), 3.10 (dd, J=15.1, 10.5 Hz, 1H), 1.62 (s, 3H). MS (ESI) m/z 776 (M+H)⁺.

### Example 78

### (7R,20S)-22-chloro-1-(4-fluorophenyl)-21-methyl-10-[(2-{3-[2-(4-methylpiperazin-1-yl)ethoxy]phenyl}pyrimidin-4-yl)methoxy]-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylic acid

### Example 78A

### ethyl (7R,20S)-18-chloro-1-(4-fluorophenyl)-19-methyl-15-[2-(4-methylpiperazin-1-yl)ethyl]-10-{[2-(methylsulfanyl)pyrimidin-4-yl]methoxy}-7,8,15,16-tetrahydro-14H-17,20-etheno-13,9-(metheno)-6-oxa-2-thia-3,5,15-triazacyclooctadeca[1,2,3-cd]indene-7-carboxylate

A mixture of Example 65M (90 mg), 4-(chloromethyl)-2-(methylthio)pyrimidine (43 mg), and cesium carbonate (161 mg) in anhydrous N,N-dimethylformamide (6 mL) was stirred at room temperature for 4 hours. The reaction mixture was partitioned between ethyl acetate and brine. The organic phase was washed with brine, and concentrated. The residue was separated by flash chromatography (0-20% methanol containing 3% NH₄OH in CH₂Cl₂) to provide the title compound. MS (ESI) m/z 868 (M+H)⁺.

### Example 78B

### ethyl (7R,20S)-18-chloro-1-(4-fluorophenyl)-19-methyl-10-[(2-{3-[2-(4-methylpiperazin-1-yl)ethoxy]phenyl}pyrimidin-4-yl)methoxy]-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylate

A mixture of Example 78A (40 mg), (3-(2-(4-methylpiperazin-1-yl)ethoxy)phenyl)boronic acid (24.33 mg), (tetrakis(triphenylphosphine)palladium(0)) (5.33 mg), and copper(I) thiophene-2-carboxylate (17.57 mg) in anhydrous tetrahydrofuran (3 mL) in a microwave vial was purged with nitrogen. The reaction mixture was heated at 90° C. under microwave irradiation (Biotage® Initiator) for 35 minutes. After cooling, the reaction mixture was partitioned between ethyl acetate and aqueous sodium bicarbonate mixture. The organic phase was washed with brine, and was concentrated. The residue was separated by flash chromatography (0-20% methanol containing 3% NH₄OH in CH₂Cl₂) to provide the title compound. MS (ESI) m/z 1041 (M+H)⁺.

### Example 78C

### (7R,20S)-22-chloro-1-(4-fluorophenyl)-21-methyl-10-[(2-{3-[2-(4-methylpiperazin-1-yl)ethoxy]phenyl}pyrimidin-4-yl)methoxy]-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylic acid

To a mixture of Example 78B (12 mg) in tetrahydrofuran (1.5 mL) was added a mixture of lithium hydroxide monohydrate (4.84 mg) in water (1.5 mL) and methanol (1.5 mL). The mixture was stirred at room temperature for 1 day, and trifluoroacetic acid (0.02 mL) was added. The mixture was concentrated, and the residue was separated by HPLC (Zorbax, C-18, 250×4.6 mm column, Mobile phase A: 0.1% trifluoroacetic acid in H₂O; B: 0.1% trifluoroacetic acid in CH₃CN; 0-70% gradient). The desired fraction was lyophilized to provide the title compound. ¹H NMR (400 MHz, dimethyl sulfoxide-d₆) δ ppm 8.71 (d, J=5.0 Hz, 1H), 8.59 (s, 1H), 7.97-7.87 (m, 2H), 7.54-7.41 (m, 3H), 7.33-7.07 (m, 7H), 6.85 (d, J=8.4 Hz, 1H), 6.52 (d, J=2.1 Hz, 1H), 5.92 (dd, J=9.2, 4.3 Hz, 1H), 5.31-5.03 (m, 4H), 4.41-4.00 (m, 8H), 3.42-2.90 (m, 20H), 2.78 (d, J=5.7 Hz, 6H), 1.75 (s, 3H). MS (ESI) m/z 1012 (M+H)⁺.

### Example 79

### (7R,21S)-19-chloro-1-(4-fluorophenyl)-10-{[2-(2-methoxyphenyl)pyrimidin-4-yl]methoxy}-20-methyl-7,8,15,16-tetrahydro-18,21-etheno-9,13-(metheno)-6,14,17-trioxa-2-thia-3,5-diazacyclononadeca[1,2,3-cd]indene-7-carboxylic acid

### Example 79A

### (R)-ethyl 2-((5-bromo-6-(4-fluorophenyl)thieno[2,3-d]pyrimidin-4-yl)oxy)-3-(5-hydroxy-2-((2-(2-methoxyphenyl)pyrimidin-4-yl)methoxy)phenyl)propanoate

A mixture of trifluoroacetic acid and water (9:1, 2.3 mL) was added to Example 68C (200 mg), and the reaction mixture was allowed to stir at room temperature. After 90 minutes, the reaction mixture was quenched slowly with saturated aqueous sodium bicarbonate and was extracted with ethyl acetate three times. The combined organic layers were dried over sodium sulfate, filtered and concentrated. The crude residue was purified by normal phase MPLC on a Teledyne Isco Combiflash Rf+ (10-80% ethyl acetate in heptanes) to provide the title compound. MS (ESI) m/z 731.2 (M+H)⁺.

### Example 79B

### (R)-ethyl 2-((5-bromo-6-(4-fluorophenyl)thieno[2,3-d]pyrimidin-4-yl)oxy)-3-(5-(2-((tert-butyldimethylsilyl)oxy)ethoxy)-2-((2-(2-methoxyphenyl)pyrimidin-4-yl)methoxy)phenyl)propanoate

To a mixture of Example 79A (169 mg) and 2-((tert-butyldimethylsilyl)oxy)ethanol (81 mg) in toluene (2.3 mL) was added triphenylphosphine (121 mg) followed by N,N,N,N-tetramethylazodicarboxamide (80 mg) and the reaction mixture was allowed to stir overnight. The reaction mixture was diluted with ethyl acetate, filtered over diatomaceous earth, and concentrated. The crude residue was purified by normal phase MPLC on a Teledyne Isco Combiflash Rf+ (10-75% ethyl acetate in heptanes) to provide the title compound. MS (ESI) m/z 891.1 (M+H)⁺.

### Example 79C

### 2-chloro-3-methyl-4-(4,4,5,5-tetramethyl-1,3,2-dioxaborolan-2-yl)phenol

Example 64C (20 g), bis(pinacolato)diboron (22.9 g), potassium acetate (17.7 g) and 1,1′-bis(diphenylphosphino)ferrocenedichloro palladium(II) dichloromethane complex (7.37 g) were combined in a 500 mL 3-neck round bottom flask equipped with a thermocouple, a reflux condenser and a stir bar. The system was degassed under a stream of nitrogen for 1 hour. Dioxane (200 mL) was added via cannula. The resulting mixture was heated to an internal temperature of 80° C. overnight. The reaction mixture was cooled and was poured into ice-water (1000 mL). Methyl-tert-butyl ether (500 mL) was added and the mixture was filtered through diatomaceous earth, rinsing with methyl tert-butyl ether. The layers were separated and the aqueous layer was extracted twice more with 500 mL methyl tert-butyl ether. The combined organic extracts were washed with water (3×500 mL) and brine (500 mL), dried over sodium sulfate, filtered, and concentrated. The residue was dissolved in 1:1 methyl tert-butyl ether-toluene and was filtered through a plug of silica, eluting with 1:1 methyl tert-butyl ether-toluene until the UV active spot finished eluting. The resulting mixture was concentrated in vacuo. The residue was triturated with heptane. The heptane mixture was successively concentrated, and the residue was dissolved in 1:1 methyl-tert-butyl ether:toluene and was triturated with heptane twice more to provide the title compound. MS (ESI) m/z 266.9 (M−H)⁻.

### Example 79D

### (2R)-ethyl 3-(5-(2-((tert-butyldimethylsilyl)oxy)ethoxy)-2-((2-(2-methoxyphenyl)pyrimidin-4-yl)methoxy)phenyl)-2-((5-((1S)-3-chloro-4-hydroxy-2-methylphenyl)-6-(4-fluorophenyl)thieno[2,3-d]pyrimidin-4-yl)oxy)propanoate

To a mixture of Example 79B (142 mg), Example 79C (51.4 mg), potassium phosphate tribasic (102 mg) and bis(di-tert-butyl(4-dimethylaminophenyl)phosphine)dichloropalladium(II) (11.30 mg) purged with nitrogen was added degassed tetrahydrofuran (1.3 mL) and water (320 μL), and the reaction mixture was stirred overnight. 1-Pyrrolidinecarbodithioic acid ammonium salt (2.62 mg) was added, and the reaction mixture was allowed to stir for 30 minutes. The reaction mixture was diluted with ethyl acetate and filtered over diatomaceous earth. Brine and water were added, and the aqueous layer was extracted with ethyl acetate three times. The combined organic layers were dried over anhydrous sodium sulfate, filtered and concentrated. The resulting residue was again subjected to the same reaction and workup conditions, and the crude residue was purified by normal phase MPLC on a Teledyne Isco Combiflash Rf+ (0-60% ethyl acetate in heptanes) to provide the title compound. MS (ESI) m/z 951.1 (M+H)⁺.

### Example 79E

### (2R)-ethyl 2-((5-((1S)-3-chloro-4-hydroxy-2-methylphenyl)-6-(4-fluorophenyl)thieno[2,3-d]pyrimidin-4-yl)oxy)-3-(5-(2-hydroxyethoxy)-2-((2-(2-methoxyphenyl)pyrimidin-4-yl)methoxy)phenyl)propanoate

To a mixture of Example 79D (75 mg) in tetrahydrofuran (525 μL) was added tetrabutylammonium fluoride (1 M in tetrahydrofuran, 158 μL), and the reaction mixture was allowed to stir. Upon consumption of the starting material, the reaction mixture was quenched with saturated aqueous ammonium chloride and water, and the aqueous mixture was extracted with ethyl acetate three times. The combined organic layers were dried over anhydrous sodium sulfate, filtered and concentrated. The crude residue was purified by normal phase MPLC on a Teledyne Isco Combiflash Rf+ (25-100% ethyl acetate in heptanes) to provide the title compound. MS (ESI) m/z 837.2 (M+H)⁺.

### Example 79F

### ethyl (7R,21S)-19-chloro-1-(4-fluorophenyl)-10-{[2-(2-methoxyphenyl)pyrimidin-4-yl]methoxy}-20-methyl-7,8,15,16-tetrahydro-18,21-etheno-13,9-(metheno)-6,14,17-trioxa-2-thia-3,5-diazacyclononadeca[1,2,3-cd]indene-7-carboxylate

To a mixture of Example 79E (51 mg) in toluene (6 mL) was added triphenylphosphine (32.0 mg) followed by tetramethylazodicarboxamide (20.98 mg), and the reaction mixture was allowed to stir at room temperature overnight. The reaction mixture was diluted with ethyl acetate, filtered over diatomaceous earth, and concentrated. The crude residue was purified by normal phase MPLC on a Teledyne Isco Combiflash Rf+ (15-80% ethyl acetate in heptanes) to provide the title compound. MS (ESI) m/z 819.3 (M+H)⁺.

### Example 79G

### (7R,21S)-19-chloro-1-(4-fluorophenyl)-10-{[2-(2-methoxyphenyl)pyrimidin-4-yl]methoxy}-20-methyl-7,8,15,16-tetrahydro-18,21-etheno-9,13-(metheno)-6,14,17-trioxa-2-thia-3,5-diazacyclononadeca[1,2,3-cd]indene-7-carboxylic acid

To a mixture of Example 79F (12.6 mg) in tetrahydrofuran (200 μL) and methanol (200 μL) was added lithium hydroxide (7.3 mg) in water (200 μL), and the reaction mixture was allowed to stir for five hours. The reaction mixture was quenched with trifluoroacetic acid (30 μL) and was diluted with water. The aqueous mixture was extracted with dichloromethane three times, and the combined organic layers were dried over anhydrous sodium sulfate, filtered and concentrated. The crude residue was taken up in dimethyl sulfoxide (700 μL) and was purified by RP-HPLC on a Gilson PLC 2020 using a Luna column (250×50 mm, 10 mm) (15-100% acetonitrile in water containing 0.1% trifluoroacetic acid) to provide the title compound after lyophilization. ¹H NMR (500 MHz, dimethyl sulfoxide-d₆) δ ppm 8.87 (d, 1H), 8.73 (s, 1H), 7.57-7.50 (m, 2H), 7.49-7.43 (m, 1H), 7.28-7.13 (m, 6H), 7.06 (dt, 1H), 6.95 (d, 1H), 6.88 (d, 1H), 6.75 (d, 1H), 6.22 (dd, 1H), 5.76 (d, 1H), 5.20-5.08 (m, 2H), 4.85-4.76 (m, 1H), 4.44-4.37 (m, 1H), 4.34-4.26 (m, 1H), 4.16-4.07 (m, 1H), 3.83 (dd, 1H), 3.77 (s, 3H), 2.94-2.86 (m, 1H), 2.17 (s, 3H). MS (ESI) m/z 791.2 (M+H)⁺.

### Example 80

### (7R,21S)-23-chloro-1-(4-fluorophenyl)-10-{[2-(2-methoxyphenyl)pyrimidin-4-yl]methoxy}-22-methyl-7,8,16,17-tetrahydro-15H-18,21-etheno-13,9-(metheno)-6,14-dioxa-2-thia-3,5,17-triazacyclononadeca[1,2,3-cd]indene-7-carboxylic acid

### Example 80A

### (R)-ethyl 2-acetoxy-3-(5-hydroxy-2-((2-(2-methoxyphenyl)pyrimidin-4-yl)methoxy)phenyl)propanoate

Example 1L (2 g), bis(pinacolato)diboron (1.151 g), [1,1′-bis(diphenylphosphino)ferrocene]dichloropalladium(II) dichloromethane (0.154 g) and potassium acetate (1.112 g) were taken up in 20 mL dioxane. The mixture was subjected to several cycles of high vacuum and nitrogen purging, and was stirred at 65° C. for 24 hours. The mixture was cooled and poured into ether, and the mixture was rinsed twice with water, and concentrated. The crude borate was taken up in 100 mL tetrahydrofuran, and to the mixture was added 30 mL pH 7 buffer mixture, and 30% H₂O₂ mixture (0.579 mL). The mixture was stirred for 3 hours. Solid Na₂S₂O₃ (3 g) was added, then NaH₂PO₄ mixture was added to pH 5, and the resulting mixture was extracted with twice 200 mL ethyl acetate. The combined extracts were washed with brine, dried over Na₂SO₄, filtered, and concentrated. The crude material was purified on a silica gel column using 5-50% ethyl acetate in heptanes as the eluent, to provide the title compound. ¹H NMR (400 MHz, dimethyl sulfoxide-d₆) δ ppm 9.01 (s, 1H), 8.92 (d, 1H), 7.55 (m, 2H), 7.45 (m, 1H), 7.16 (d, 1H), 7.06, (t, 1H), 6.89, (d, 1H), 6.60 (m, 2H), 5.15, (m, 3H), 4.06 (q, 2H), 3.77 (s, 3H), 3.21 (dd, 1H), 3.03 (dd, 1H), 2.01, (s, 3H), 1.11 (s, 3H). LC/MS (APCI) m/z 467.3 (M+H)⁺.

### Example 80B

### (R)-ethyl 2-acetoxy-3-(2-((2-(2-methoxyphenyl)pyrimidin-4-yl)methoxy)-5-((triisopropylsilyl)oxy)phenyl)propanoate

Example 80A (1.4 g), triisopropylsilyl chloride (0.954 mL), and imidazole (0.347 g) were stirred in 20 mL N,N-dimethylformamide for 24 hours at 45° C. overnight. The reaction mixture was cooled, and poured into ether. The organics were washed three times with water and brine, dried over Na₂SO₄, filtered, and concentrated. The crude material was purified on a silica gel column using 10-40% ethyl acetate in heptanes as eluent, to provide the title compound. ¹H NMR (400 MHz, dimethyl sulfoxide-d₆) δ 9.01 ppm (s, 1H), 8.93 (d, 1H), 7.57 (d, 1H), 7.54 (d, 1H), 7.45 (dd, 1H), 7.15 (d, 1H), 7.04, (t, 1H), 6.96 (d, 1H), 6.77 (d, 1H), 5.17 (d, 1H), 5.15 (m, 2H), 4.06 (q, 2H), 3.76 (s, 3H), 3.25 (dd, 1H), 3.03 (dd, 1H), 1.99, (s, 3H), 1.01-1.27 (m, 24H). LC/MS (APCI) m/z 623.2 (M+H)⁺.

### Example 80C

### methyl (R)-2-hydroxy-3-(2-((2-(2-methoxyphenyl)pyrimidin-4-yl)methoxy)-5-((triisopropylsilyl)oxy)phenyl)propanoate

Example 80B (2.6 g) and LiOH—H₂O (0.772 g) in 70 mL tetrahydrofuran and 20 mL water were stirred overnight. The mixture was acidified with 1M aqueous HCl and was extracted with twice 200 mL ethyl acetate. The combined extracts were rinsed with brine, dried over Na₂SO₄, filtered, and concentrated. The crude material was taken up in 100 mL 1:1 methanol/ethyl acetate. Trimethylsilyldiazomethane (4.60 mL, 2M in ether) was added. The reaction mixture was stirred for 10 minutes and was concentrated. The crude material was used directly in the next step. LC/MS (APCI) m/z 567.3 (M+H)⁺.

### Example 80D

### 4-bromo-N-(2-((tert-butyldimethylsilyl)oxy)ethyl)-2-chloro-3-methylaniline

Example 7G (8.4 g), 2-((tert-butyldimethylsilyl)oxy)acetaldehyde (7.97 g), and sodium triacetoxyborohydride (11.30 g) were stirred in 200 mL dichloromethane overnight. The mixture was diluted with 400 mL ethyl acetate, washed twice with water, washed with brine, dried over Na₂SO₄, filtered, and concentrated. The crude material was purified on a silica gel column using 10% ethyl acetate in heptanes as the eluent, to provide the title compound. ¹H NMR (400 MHz, dimethyl sulfoxide-d₆) δ ppm 7.43 (d, 1H), 6.69 (d, 1H), 5.35 (t, 1H), 3.67 (t, 2H), 3.32 (dt, 2H), 2.59 (s, 3H), 0.95 (s, 9H), 0.12 (s, 6H). LC/MS (APCI) m/z 263.1 (M+CH₃CN+H)⁺.

### Example 80E

### N-(2-((tert-butyldimethylsilyl)oxy)ethyl)-2-chloro-3-methyl-4-(4,4,5,5-tetramethyl-1,3,2-dioxaborolan-2-yl)aniline

Example 80D (8 g), bis(pinacolato)diboron (6.97 g), [1,1′-bis(diphenylphosphino)ferrocene]dichloropalladium(II) dichloromethane (0.68 g) and potassium acetate (6.22 g) were taken up in 120 mL dioxane and the mixture was subjected to several cycles of high vacuum and nitrogen purging. The mixture was stirred at 65° C. for 24 hours. The mixture was cooled and poured into ethyl acetate, and the mixture was rinsed twice with water, and concentrated. The crude material was purified on a silica gel column using 1-10% ethyl acetate in heptanes as eluent, to yield the title compound. ¹H NMR (400 MHz, dimethyl sulfoxide-d₆) δ ppm 7.43 (d, 1H), 6.58 (d, 1H), 5.46 (t, 1H), 3.74 (t, 2H), 3.25 (dt, 2H), 2.46 (s, 3H), 1.25 (s, 6H), 1.15 (s, 6H), 0.84 (s, 9H), 0.01 (s, 6H). LC/MS (APCI) m/z 426.3 (M+H)⁺.

### Example 80F

### N-(2-((tert-butyldimethylsilyl)oxy)ethyl)-2-chloro-4-(4-chloro-6-(4-fluorophenyl)thieno[2,3-d]pyrimidin-5-yl)-3-methylaniline

Example 1D (1.775 g), Example 80E (2 g), bis(di-tert-butyl(4-dimethylaminophenyl)phosphine)dichloropalladium(II) (0.333 g) and potassium phosphate (2.492 g) were subjected to several vacuum/nitrogen flush cycles. Dioxane/water (40 mL of a 7:1 mixture) was added and the mixture was subjected to several more vacuum/nitrogen flush cycles. The reaction mixture was stirred for two days. The mixture was diluted with 200 mL ethyl acetate, washed with water, dried over Na₂SO₄, filtered, and concentrated. The crude material was purified on a silica gel column using 10-30% ethyl acetate in heptanes as eluent, to yield the title compound. ¹H NMR (400 MHz, dimethyl sulfoxide-d₆) δ ppm 8.95 (s, 1H), 7.36 (dd, 2H), 7.21 (dd, 2H), 6.96 (d, 1H), 6.65 (d, 1H), 5.32 (t, 1H), 3.78 (t, 2H), 3.25 (dt, 2H), 1.99 (s, 3H), 0.85 (s, 9H), 0.00 (s, 6H). LC/MS (APCI) m/z 562.1 (M+H)⁺.

### Example 80G

### (2R)-methyl 2-((5-(3-chloro-4-((2-hydroxyethyl)amino)-2-methylphenyl)-6-(4-fluorophenyl)thieno[2,3-d]pyrimidin-4-yl)oxy)-3-(5-hydroxy-2-((2-(2-methoxyphenyl)pyrimidin-4-yl)methoxy)phenyl)propanoate

Example 80F (115 mg), Example 80C (127 mg), and Cs₂CO₃ (120 mg) were stirred in 4 mL anhydrous tert-butanol at 65° C. for five days. The mixture was diluted with 100 mL ethyl acetate, washed with brine, dried over Na₂SO₄, filtered, and concentrated. The crude material contained a mixture of ester and acid products. The crude material was taken up in 50 mL 1:1 methanol/ethyl acetate, and trimethylsilyldiazomethane (1.5 mL, 2M in ether) was added. The reaction mixture was stirred for 10 minutes and was concentrated. The crude material was taken up in 50 mL tetrahydrofuran, and tetrabutyl ammonium fluoride (2 mL, 1M in tetrahydrofuran) was added. The reaction mixture was stirred for 10 minutes. The mixture was diluted with 200 mL ethyl acetate, washed with twice water, washed with brine, dried over Na₂SO₄, filtered, and concentrated. The crude material was purified on a silica gel column using 10-50% ethyl acetate in heptanes as the eluent, to yield the title compound. ¹H NMR (400 MHz, dimethyl sulfoxide-d₆) δ ppm 8.91 (m, 2H), 8.57 (s, 1H), 7.57 (d, 1H), 7.47 (d, 1H), 7.37 (m, 2H), 7.23 (dd, 2H), 7.15 (dd, 2H), 7.04 (m, 2H), 6.82 (dd, 2H), 6.67 (m, 2H), 5.47 (t, 1H), 5.22 (t, 1H), 5.15 (m, 2H), 4.82, (t, 1H), 3.77 (s, 3H), 3.76 (s, 3H), 3.60 (s, 2H), 3.58 (m, 2H), 3.17 (dd, 1H), 3.09 (dd, 1H), 1.99 (s, 3H). LC/MS (APCI) m/z 822.1 (M+H)⁺.

### Example 80H

### (7R,21S)-23-chloro-1-(4-fluorophenyl)-10-{[2-(2-methoxyphenyl)pyrimidin-4-yl]methoxy}-22-methyl-7,8,16,17-tetrahydro-15H-18,21-etheno-13,9-(metheno)-6,14-dioxa-2-thia-3,5,17-triazacyclononadeca[1,2,3-cd]indene-7-carboxylic acid

Triphenylphosphine (62.2 mg) and diethyl azodicarboxylate (94 μL) were stirred together in 2 mL tetrahydrofuran for 10 minutes. Half of the mixture was added to Example 80G (65 mg) in 2 mL tetrahydrofuran, and the mixture was stirred overnight. Water (1 mL) was added, LiOH—H₂O (1.9 mg) was added and the mixture was stirred overnight. The mixture was then taken up in 50 mL dichloromethane, and 4 mL aqueous NaH₂PO₄ was added. The layers were separated, and the organic layer was dried over Na₂SO₄, filtered, and concentrated. The residue was dissolved in dimethylformamide and was purified on a Grace Reveleris X2 MPLC using a Phenomenex® Luna™ 10 M 150×30 mm C18 column eluting with a gradient over 40 minutes of 15% to 75% acetonitrile/0.1% trifluoroacetic acid in water. The product containing fractions were combined, and free-based by adding 1 mL aqueous Na₂CO₃. The aqueous layer was extracted twice with dichloromethane, and the organic layer was dried over Na₂SO₄, filtered, and concentrated to provide the title compound. ¹H NMR (400 MHz, dimethyl sulfoxide-d₆) δ ppm 14.70 (br s, 1H), 8.83 (s, 1H), 8.42 (s, 1H), 7.58 (d, 1H), 7.49 (m, 2H), 7.39 (m, 1H), 7.30 (d, 1H), 7.13 (m, 4H), 7.01 (d, 1H), 6.73 (dd, 2H), 6.59 (m, 2H), 5.47 (t, 1H), 5.13 (m, 1H), 4.32 (m, 2H), 3.75 (m, 2H), 3.69 (s, 3H), 3.53 (dd, 1H), 3.10 (m, 1H), 2.33 (m, 1H), 2.13 (m, 1H), 1.74 (s, 3H). MS (ESI) m/z 790.0 (M+H)⁺.

### Example 81

### (7R,21S)-23-chloro-1-(4-fluorophenyl)-10-{[2-(2-methoxyphenyl)pyrimidin-4-yl]methoxy}-22-methyl-17-[2-(morpholin-4-yl)ethyl]-7,8,16,17-tetrahydro-15H-18,21-etheno-13,9-(metheno)-6,14-dioxa-2-thia-3,5,17-triazacyclononadeca[1,2,3-cd]indene-7-carboxylic acid

### Example 81A

### N-(2-chloro-3-methylphenyl)-2-morpholinoacetamide

2-Chloro-3-methylaniline (20 g), 2-morpholinoacetic acid (22.55 g), 1-[bis(dimethylamino)methylene]-1H-1,2,3-triazolo[4,5-b]pyridinium 3-oxide hexafluorophosphate (HATU, 61.8 g) and N,N-diisopropylethylamine (29.6 mL) were taken up in 200 mL N,N-dimethylformamide at 0° C. The mixture was warmed to room temperature and was stirred overnight. The mixture was taken up in 2 L water, and was extracted three times with 500 mL ethyl acetate. The combined extracts were washed three times with water, washed with brine, dried over Na₂SO₄, filtered, and concentrated to provide the title compound. LC/MS (APCI) m/z 269.2 (M+H)⁺.

### Example 81B

### N-(2-((tert-butyldimethylsilyl)oxy)ethyl)-N-(2-chloro-3-methylphenyl)-2-morpholinoacetamide

NaH (0.179 g, 60% in mineral oil) was added to Example 81A (1 g) in 12 mL N,N-dimethylformamide and the mixture was stirred for 30 minutes. (2-Bromoethoxy)(tert-butyl)dimethylsilane (1.068 g) was added, and the reaction mixture was stirred for 24 hours. The mixture was taken up in 300 mL ethyl acetate, washed three times with water, washed with brine, dried over Na₂SO₄, filtered, and concentrated. The crude material was purified on a silica gel column using 10-50% ethyl acetate in heptanes as eluent, to provide the title compound. ¹H NMR (400 MHz, dimethyl sulfoxide-d₆) δ ppm 7.48 (dd, 1H), 7.41 (m, 2H), 4.19 (m, 1H), 3.81 (m, 1H), 3.70 (m, 1H), 3.53 (m, 4H), 3.20, (m, 1H), 2.88 (q, 2H), 2.49 (s, 3H), 2.32 (t, 4H), 0.89 (s, 6H), 0.08 (s, 9H). LC/MS (APCI) m/z 427.3 (M+H)⁺.

### Example 81C

### 2-((2-chloro-3-methylphenyl)(2-morpholinoethyl)amino)ethanol

Borane-tetrahydrofuran (72 mL, 1M in tetrahydrofuran) was added to Example 81B (11 g) in 50 mL tetrahydrofuran and the mixture was stirred for two days at 45° C. The mixture was cooled with ice water, and methanol was added slowly via syringe until gas evolution ceased (˜30 mL). The resulting mixture was poured into 200 mL 1M aqueous HCl, and the mixture was stirred overnight. Saturated aqueous Na₂CO₃ was added until the mixture was basic. The reaction mixture was extracted three times with ethyl acetate. The combined extracts were washed with brine, dried over Na₂SO₄, filtered, and concentrated. The crude material was purified on a silica gel column using 10-50% ethyl acetate in heptanes as eluent, to provide the title compound. ¹H NMR (400 MHz, dimethyl sulfoxide-d₆) δ ppm 7.19 (m, 2H), 7.15 (dd, 1H), 4.51 (br s, 1H), 3.54 (m, 4H), 3.47 (t, 2H), 3.27 (t, 2H), 3.18 (t, 2H), 2.36 (m, 9H). LC/MS (APCI) m/z 299.2 (M+H)⁺.

### Example 81D

### 2-((4-bromo-2-chloro-3-methylphenyl)(2-morpholinoethyl)amino)ethanol

Example 81C (3.8 g) and ammonium acetate (0.098 g) were stirred in 90 mL acetonitrile at 0° C., and N-bromosuccinimide (2.490 g) was added in three portions over 10 minutes. The reaction mixture was allowed to warm to room temperature overnight. Saturated sodium thiosulfate mixture (20 mL) was added, and the mixture was extracted twice with ethyl acetate. The combined extracts were washed with brine, dried over Na₂SO₄, filtered, and concentrated. The crude material was purified on a silica gel column using 10-100% ethyl acetate in heptanes, followed by 5% methanol in ethyl acetate with 1% trimethylamine, as eluent, to provide the title compound. ¹H NMR (400 MHz, dimethyl sulfoxide-d₆) δ ppm 7.49 (d, 1H), 7.12 (d, 1H), 4.49 (br s, 1H), 3.48 (m, 4H), 3.42 (t, 2H), 3.24 (t, 2H), 3.15 (t, 2H), 2.45 (s, 3H), 2.30 (m, 6H). LC/MS (APCI) m/z 379.1 (M+H)⁺.

### Example 81E

### 2-((2-chloro-3-methyl-4-(4,4,5,5-tetramethyl-1,3,2-dioxaborolan-2-yl)phenyl)(2-morpholinoethyl)amino)ethanol

Example 81D (1.9 g), bis(pinacolato)diboron (1.66 g), [1,1′-bis(diphenylphosphino)ferrocene]dichloropalladium(II) dichloromethane (0.288 g) and potassium acetate (1.48 g) were taken up in 25 mL dioxane and were subjected to several cycles of high vacuum and nitrogen purging, and were stirred at 70° C. for 24 hours. The crude material was purified on a silica gel column using 0-5% methanol in ethyl acetate with 1% triethylamine as eluent, to yield the title compound. ¹H NMR (400 MHz, dimethyl sulfoxide-d₆) δ ppm 7.51 (d, 1H), 7.12 (d, 1H), 4.49 (br s, 1H), 3.49 (m, 4H), 3.44 (m, 2H), 3.28 (t, 2H), 3.19 (t, 2H), 2.50 (s, 3H), 2.31 (m, 6H), 1.44 (s, 12H). LC/MS (APCI) m/z 425.1 (M+H)⁺.

### Example 81F

### (R)-methyl 2-((5-bromo-6-(4-fluorophenyl)thieno[2,3-d]pyrimidin-4-yl)oxy)-3-(2-((2-(2-methoxyphenyl)pyrimidin-4-yl)methoxy)-5-((triisopropylsilyl)oxy)phenyl)propanoate

Example 1D (1.67 g), Example 80C (2.3 g) and Cs₂CO₃ (2.380 g) were stirred in 25 mL anhydrous tert-butanol at 65° C. overnight. The mixture was cooled, poured into ethyl acetate, washed twice with water, dried over Na₂SO₄, filtered, and concentrated. The crude material was purified on a silica gel column using 10-30% ethyl acetate in heptanes as the eluent to provide the title compound. ¹H NMR (400 MHz, dimethyl sulfoxide-d₆) δ ppm 8.91 (d, 1H), 8.62 (s, 1H), 7.71 (m, 2H), 7.61 (d, 1H), 7.51 (d, 1H), 7.43 (m, 3H), 7.13 (d, 1H), 7.03, (t, 1H), 6.98 (d, 1H), 6.92 (d, 1H), 6.69 (dd, 1H), 5.90 (d, 1H), 5.20 (q, 2H), 3.75 (s, 3H), 3.73 (s, 3H), 3.62 (dd, 1H), 3.24 (dd, 1H), 1.99, (s, 3H), 1.21 (m, 3H), 0.88 (m, 18H). LC/MS (APCI) m/z 873.1 (M+H)⁺.

### Example 81G

### (R)-methyl 2-((5-bromo-6-(4-fluorophenyl)thieno[2,3-d]pyrimidin-4-yl)oxy)-3-(5-hydroxy-2-((2-(2-methoxyphenyl)pyrimidin-4-yl)methoxy)phenyl)propanoate

Example 81F (1.0 g) was stirred in 15 mL tetrahydrofuran and tetrabutyl ammonium fluoride (tetra-n-butylammonium fluoride, 1.144 mL, 1M in tetrahydrofuran) was added dropwise and the reaction mixture was stirred for 10 minutes. The reaction mixture was poured into ethyl acetate, washed with water and brine, dried over Na₂SO₄, filtered, and concentrated. The crude material was purified on a silica gel column using 10-100% ethyl acetate in heptanes as eluent, to yield the title compound. LC/MS (APCI) m/z 718.9 (M+H)⁺.

### Example 81H

### (2R)-methyl 2-((5-((1S)-3-chloro-4-((2-hydroxyethyl)(2-morpholinoethyl)amino)-2-methylphenyl)-6-(4-fluorophenyl)thieno[2,3-d]pyrimidin-4-yl)oxy)-3-(5-hydroxy-2-((2-(2-methoxyphenyl)pyrimidin-4-yl)methoxy)phenyl)propanoate

Example 81G (400 mg), Example 81E (237 mg), [1,1′-bis(diphenylphosphino)ferrocene]dichloropalladium(II) dichloromethane (39.5 mg) and potassium phosphate (355 mg) were placed in a 5 mL pressure vial and the mixture was repeatedly degassed and purged with nitrogen. Tetrahydrofuran (2 mL) and water (0.5 mL) were added via syringe and the mixture was repeatedly degassed and purged with nitrogen. The reaction mixture was stirred overnight. The crude material was purified on a silica gel column using 0-10% methanol in ethyl acetate with 1% triethylamine as eluent, to provide the title compound. ¹H NMR (400 MHz, dimethyl sulfoxide-d₆) δ ppm 8.94 (m, 2H), 8.67 (s, 1H), 7.55 (m, 4H), 7.41 (m, 2H), 7.25 (m, 5H), 7.17 (dd, 1H), 6.92 (dd, 1H), 6.55 (d, 1H), 5.49 (t, 1H), 5.16 (q, 2H), 4.52 (br s, 1H), 3.81 (s, 3H), 3.56 (s, 3H), 3.46 (m, 4H), 3.42 (m, 2H), 3.27 (t, 2H), 3.20 (t, 2H), 2.89 (m, 1H), 2.66 (m, 1H), 2.39 (m, 2H), 2.24 (m, 4H), 2.01 (s, 3H). LC/MS (APCI) m/z 934.9 (M+H)⁺.

### Example 81I

### (7R,21S)-23-chloro-1-(4-fluorophenyl)-10-{[2-(2-methoxyphenyl)pyrimidin-4-yl]methoxy}-22-methyl-17-[2-(morpholin-4-yl)ethyl]-7,8,16,17-tetrahydro-15H-18,21-etheno-13,9-(metheno)-6,14-dioxa-2-thia-3,5,17-triazacyclononadeca[1,2,3-cd]indene-7-carboxylic acid

Triphenylphosphine (101 mg) and diethyl azodicarboxylate (152 μL) were stirred together in 2 mL tetrahydrofuran for 10 minutes, at which point half of the mixture was added to Example 81H (120 mg) in 2 mL tetrahydrofuran. The mixture was stirred overnight. Water (1 mL) was added, then LiOH—H₂O (15.3 mg) was added and the mixture was stirred overnight. The mixture was taken up in 250 mL dichloromethane, and 4 mL aqueous NaH₂PO₄ was added. The layers were separated, and the organic layer was dried over Na₂SO₄, filtered, and concentrated. The residue was dissolved in dimethylformamide and was purified on a Grace Reveleris X2 MPLC using a Phenomenex® Luna™ 10 M 150×30 mm C18 column eluting with a gradient over 55 minutes of 25% to 65% acetonitrile/0.1% trifluoroacetic acid in water. The product-containing fractions were combined and free-based by adding 1 mL aqueous Na₂CO₃. The aqueous layer was extracted twice with dichloromethane, and the combined extracts were dried over Na₂SO₄. Filtration and concentration of the filtrate provided the title compound. ¹H NMR (400 MHz, dimethyl sulfoxide-d₆) δ ppm 10.47 (br s, 1H), 8.90 (s, 1H), 8.76 (s, 1H), 7.57 (m, 3H), 7.47 (m, 1H), 7.26 (m, 1H), 7.18 (m, 4H), 7.07 (m, 1H), 6.98 (m, 1H), 6.89 (m, 1H), 6.79 (s, 1H), 6.17 (s, 1H), 5.70 (s, 1H), 5.16 (q, 2H), 4.44 (m, 1H), 4.15 (s, 1H), 4.05 (s, 1H), 3.98-3.60 (m, 5H), 3.77 (s, 3H), 3.50 (m, 2H), 3.23 (d, 2H), 3.14 (m, 2H), 2.94 (m, 1H), 2.68 (m, 1H), 2.21 (m, 2H), 1.99 (s, 3H). LC/MS (APCI) m/z 903.4 (M+H)⁺.

### Example 82

### (7R,16R,21S)-19-chloro-1-(4-fluorophenyl)-16-({4-[2-(methanesulfonyl)ethyl]piperazin-1-yl}methyl)-10-{[2-(2-methoxyphenyl)pyrimidin-4-yl]methoxy}-20-methyl-7,8,15,16-tetrahydro-18,21-etheno-13,9-(metheno)-6,14,17-trioxa-2-thia-3,5-diazacyclononadeca[1,2,3-cd]indene-7-carboxylic acid

### Example 82A

### ethyl (7R,16R,21S)-19-chloro-1-(4-fluorophenyl)-16-({4-[2-(methanesulfonyl)ethyl]piperazin-1-yl}methyl)-10-{[2-(2-methoxyphenyl)pyrimidin-4-yl]methoxy}-20-methyl-7,8,15,16-tetrahydro-18,21-etheno-9,13-(metheno)-6,14,17-trioxa-2-thia-3,5-diazacyclononadeca[1,2,3-cd]indene-7-carboxylate

Example 82A was prepared according to the procedure described for Example 73J, substituting 1-[2-(methylsulfonyl)ethyl]piperazine for 1-methylpiperazine. LC/MS (APCI) m/z 1023.2 (M+H)⁺.

### Example 82B

### (7R,16R,21S)-19-chloro-1-(4-fluorophenyl)-16-({4-[2-(methanesulfonyl)ethyl]piperazin-1-yl}methyl)-10-{[2-(2-methoxyphenyl)pyrimidin-4-yl]methoxy}-20-methyl-7,8,15,16-tetrahydro-18,21-etheno-13,9-(metheno)-6,14,17-trioxa-2-thia-3,5-diazacyclononadeca[1,2,3-cd]indene-7-carboxylic acid

Example 82A (140 mg) was dissolved in methanol (0.9 mL) and tetrahydrofuran (1.8 mL), and to the resulting stirred mixture was slowly added 1 molar aqueous lithium hydroxide (2.0 mL). The reaction mixture was stirred at ambient temperature overnight. The mixture was concentrated to remove the volatiles, and the aqueous mixture was treated with acetic acid until pH was slightly acidic. The precipitate that was formed was dissolved by the addition of 2 mL of acetonitrile. The mixture was purified by reverse phase prep LC using a Gilson 2020 system (Luna, C-18, 250×50 mm column, mobile phase A: 0.1% trifluoroacetic acid in water; B: acetonitrile; 5-75% B to A gradient at 70 mL/min) to provide the title compound. ¹H NMR (400 MHz, dimethyl sulfoxide-d₆) δ ppm 2.23 (s, 3H), 2.33-2.47 (m, 8H), 2.54-2.63 (m, 2H), 2.67 (t, J=6.7 Hz, 2H), 2.88 (d, J=16.9 Hz, 1H), 3.01 (s, 3H), 3.19-3.28 (m, 2H), 3.77 (s, 3H), 3.83-3.93 (m, 1H), 4.31 (dd, J=13.2, 8.6 Hz, 1H), 4.48 (d, J=12.9 Hz, 1H), 4.52-4.63 (m, 1H), 5.17 (q, J=15.1 Hz, 2H), 5.61-5.70 (m, 1H), 6.13 (dd, J=5.3, 2.9 Hz, 1H), 6.78 (dd, J=9.0, 2.9 Hz, 1H), 6.90 (d, J=9.0 Hz, 1H), 6.95 (d, J=8.3 Hz, 1H), 7.06 (td, J=7.4, 1.0 Hz, 1H), 7.11-7.25 (m, 6H), 7.43-7.50 (m, 1H), 7.50-7.58 (m, 2H), 8.73 (s, 1H), 8.88 (d, J=5.1 Hz, 1H). LC/MS (APCI) m/z 995.2 (M+H)⁺.

### Example 83

### (7R,20S)-18-chloro-1-(4-fluorophenyl)-10-({2-[3-(2-methoxyethyl)oxetan-3-yl]pyrimidin-4-yl}methoxy)-19-methyl-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylic acid

### Example 83A

### ethyl 2-(oxetan-3-ylidene)acetate

To a mixture of 3-oxetanone (1 mL) in dichloromethane (31.2 mL) was added (carbethoxymethylene)triphenylphosphorane (5.98 g) at 0° C. The mixture was allowed to warm to room temperature over 16 hours and was concentrated. The mixture was filtered through 24 g silica gel (2:1 heptanes/ethyl acetate) to provide the title compound. ¹H NMR (400 MHz, chloroform-d): δ ppm 5.60 (m, 1H), 5.47 (m, 2H), 5.27 (m, 2H), 4.13 (q, J=7.1 Hz, 2H), 1.24 (t, J=7.1 Hz, 3H). LC/MS (APCI) m/z 143.2 (M+H)⁺.

### Example 83B

### ethyl 2-(3-cyanooxetan-3-yl)acetate

To a mixture of Example 83A (1.32 g) in acetonitrile (93 mL) was added acetone cyanohydrin (1.696 mL), potassium cyanide (1.209 g), and 18-crown-6 (4.91 g) at room temperature. After stirring for 18 hours, the mixture was concentrated in vacuo and the residue was purified by silica gel flash chromatography (4:1 heptanes/ethyl acetate) to provide the title compound. ¹H NMR (400 MHz, chloroform-d): δ ppm 5.01 (d, J=6.6 Hz, 2H), 4.55 (d, J=6.6 Hz, 2H), 4.22 (q, J=7.1 Hz, 2H), 3.08 (s, 2H), 1.29 (t, J=7.2 Hz, 3H).

### Example 83C

### 3-(2-hydroxyethyl)oxetane-3-carbonitrile

N-Butyllithium in hexane (2.483 mL, 2.5 M in THF) was added to a mixture of diisobutylaluminum hydride (6.21 mL, 1M in THF) in anhydrous tetrahydrofuran (14.78 mL) at 0° C. and the mixture was stirred for 30 minutes. A mixture of Example 83B (0.5 g) in dry tetrahydrofuran (15 mL) at −78° C. was treated with the ate complex over a period of 1 hour. The reaction mixture was then stirred at −78° C. for 3 hours, after which a mixture of sodium borohydride (0.291 g) in absolute ethanol (7.5 mL) was added dropwise. The mixture was allowed to warm to room temperature over 1 hour, and was neutralized with aqueous hydrochloric acid (1M). The mixture was extracted with ethyl acetate. The organic layer was washed with saturated aqueous sodium bicarbonate followed by brine, and concentrated. The crude product was purified by flash column chromatography on a 24 g silica gel column (0-5% methanol/dichloromethane) to provide the title compound. ¹H NMR (400 MHz, chloroform-d) δ ppm 4.98 (d, J=6.3 Hz, 2H), 4.68 (d, J=6.3 Hz, 2H), 3.95 (td, J=5.8, 3.7 Hz, 2H), 2.29 (t, J=5.9 Hz, 2H), 1.51 (t, J=4.2 Hz, 1H).

### Example 83D

### 3-(2-((tert-butyldimethylsilyl)oxy)ethyl)oxetane-3-carbonitrile

Example 83C (230 mg) was dissolved in anhydrous dichloromethane (2.4 mL). Imidazole (160 mg) and tert-butyldimethylsilyl chloride (230 mg) were added and the resulting reaction mixture was stirred for 20 hours at room temperature. The mixture was quenched with water (5 mL) and was extracted with dichloromethane (3×5 mL). The combined organic phase was washed with brine (10 mL) and water (10 mL), dried over MgSO₄, filtered, and concentrated. The title compound was isolated via flash chromatography (0-10% ethyl acetate/heptanes). ¹H NMR (500 MHz, chloroform-d) δ ppm 4.93 (d, J=6.3 Hz, 2H), 4.67 (d, J=6.3 Hz, 2H), 3.87 (t, J=5.6 Hz, 2H), 2.21 (t, J=5.7 Hz, 2H), 0.88 (s, 9H), 0.07 (s, 6H). LC/MS (APCI) m/z 242.4 (M+H)⁺.

### Example 83E

### 3-(2-((tert-butyldimethylsilyl)oxy)ethyl)oxetane-3-carboximidamide

A 2 M mixture of trimethylaluminum in toluene (1.01 mL) was slowly added to a magnetically stirred suspension of ammonium chloride (109 mg) in toluene (3.8 mL) at 0° C. under a nitrogen atmosphere. After the addition, the mixture was warmed to 25° C. and was stirred for 2 hours until gas evolution had ceased. Example 83D (273 mg) in toluene (1.9 mL) was added and the mixture was heated to 80° C. for 12 hours under nitrogen. The mixture was cooled down to 0° C., quenched carefully with 10 mL methanol, and stirred at 20° C. for 2 hours. The material was filtered and washed with methanol several times. The filtrate was concentrated under vacuum to provide the title compound which was used without further purification. LC/MS (APCI) m/z 259.4 (M+H)⁺.

### Example 83F

### 2-(3-(4-(dimethoxymethyl)pyrimidin-2-yl)oxetan-3-yl)ethanol

Example 83E (0.292 g) and (E)-4-(dimethylamino)-1,1-dimethoxybut-3-en-2-one (0.392 g) were taken up in methanol (3.77 mL), and sodium methoxide (0.367 g) was added in portions. The mixture was heated at 80° C. for 20 hours. The reaction mixture was cooled and concentrated. The residue was mixed with ethyl acetate (15 mL), and water was added carefully (20 mL). The mixture was stirred for 15 minutes to dissolve all the material. The mixture was extracted with ethyl acetate. The combined organic layers were washed with brine, dried with Na₂SO₄, filtered, and concentrated. The crude material was purified by silica gel flash chromatography (10-50% ethyl acetate/heptanes) to provide the title compound. ¹H NMR (400 MHz, chloroform-d) δ ppm 8.76 (d, J=5.0 Hz, 1H), 7.43 (d, J=5.1 Hz, 1H), 4.31 (dd, J=7.6, 5.9 Hz, 1H), 4.19-4.03 (m, 4H), 3.98 (dd, J=11.3, 5.8 Hz, 1H), 3.90 (dd, J=11.3, 7.5 Hz, 1H), 3.45 (t, J=0.9 Hz, 6H), 2.50 (ddd, J=12.6, 8.0, 6.3 Hz, 1H), 2.13 (dt, J=12.6, 7.0 Hz, 1H). LC/MS (APCI) m/z 255.4 (M+H)⁺.

### Example 83G

### 4-(dimethoxymethyl)-2-(3-(2-methoxyethyl)oxetan-3-yl)pyrimidine

Example 83F (90 mg) was dissolved in tetrahydrofuran (1.1 mL). Sodium hydride (18.40 mg) was added to the mixture at 0° C. After 20 minutes, iodomethane (44.1 μL) was added to the reaction mixture and the mixture was stirred at 35° C. for 18 hours. The reaction mixture was cooled in an ice bath, quenched with saturated sodium bicarbonate mixture (5 mL), and extracted with dichloromethane (3×10 mL). The combined organic layer was concentrated. The crude product was purified by silica gel chromatography (10-50% ethyl acetate/heptanes) to provide the title compound. LC/MS (APCI) m/z 269.3 (M+H)⁺.

### Example 83H

### (2-(3-(2-methoxyethyl)oxetan-3-yl)pyrimidin-4-yl)methanol

At room temperature, aqueous 2N hydrochloric acid mixture (1.1 mL) was mixed with Example 83G (95 mg) in a 20 mL vial and the mixture was stirred at 60° C. for 3 hours. The reaction mixture was cooled to room temperature and 1,4-dioxane (1.2 mL) was added. The mixture was further cooled to 0° C. Powdered sodium hydroxide (85 mg) was added in portions over about 10 minutes. The reaction mixture was stirred until all the solid sodium hydroxide was dissolved. Sodium hydroxide mixture (1N) was added until the pH was adjusted to around 8. Solid sodium borohydride (26.8 mg, 0.708 mmol) was added to the mixture all at once. The reaction mixture was stirred at 0° C. for 1 hour, quenched with water, stirred for another 30 minutes, and extracted with dichloromethane. The combined organic layer was concentrated and subjected to column chromatography (50-100% ethyl acetate/heptanes) to provide the title compound. ¹H NMR (400 MHz, chloroform-d) δ ppm 8.66 (d, J=5.1 Hz, 1H), 7.12 (dd, J=5.2, 0.8 Hz, 1H), 4.75 (d, J=4.3 Hz, 2H), 4.29 (d, J=9.0 Hz, 1H), 4.07-3.96 (m, 2H), 3.91 (td, J=8.3, 6.6 Hz, 1H), 3.80 (s, 2H), 3.49 (t, J=5.0 Hz, 1H), 3.28 (s, 3H), 2.62 (ddd, J=12.6, 8.1, 5.9 Hz, 1H), 2.20 (ddd, J=12.7, 8.0, 6.7 Hz, 1H). LC/MS (APCI) m/z 225.3 (M+H)⁺.

### Example 83I

### 4-(chloromethyl)-2-(3-(2-methoxyethyl)oxetan-3-yl)pyrimidine

To a mixture of Example 83H (40 mg) in anhydrous dichloromethane (1.8 mL) was added triphenylphosphine (60.8 mg) at 0° C. The mixture was stirred at 0° C. for 45 minutes, and N-chlorosuccinimide (26.2 mg) was added. The reaction mixture was allowed to warm to room temperature for 2 hours. The reaction mixture was directly loaded onto a 12 g silica gel column that was eluted with 0-50% ethyl acetate in heptanes to provide the title compound. ¹H NMR (501 MHz, chloroform-d) δ ppm 8.75 (d, J=5.0 Hz, 1H), 7.39 (d, J=5.1 Hz, 1H), 4.61 (s, 2H), 4.28 (d, J=9.0 Hz, 1H), 4.05-3.95 (m, 2H), 3.90 (q, J=7.7 Hz, 1H), 3.79 (d, J=2.4 Hz, 2H), 3.27 (d, J=1.2 Hz, 3H), 2.62 (ddd, J=13.3, 8.2, 6.0 Hz, 1H), 2.18 (dt, J=13.2, 7.4 Hz, 1H). LC/MS (APCI) m/z 243.3 (M+H)⁺.

### Example 83J

### (7R,20S)-18-chloro-1-(4-fluorophenyl)-10-({2-[3-(2-methoxyethyl)oxetan-3-yl]pyrimidin-4-yl}methoxy)-19-methyl-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylic ethyl ester

A mixture of Example 65M (55 mg), Example 83I (36.6 mg), and cesium carbonate (98 mg) in anhydrous dimethylformamide (2.5 mL) was stirred at room temperature for 16 hours. The reaction mixture was partitioned between ethyl acetate and brine. The organic phase was separated and concentrated. The residue was separated by flash chromatography (0-20% methanol/dichloromethane containing 1% triethylamine) to provide the title compound. LC/MS (APCI) m/z 936.1 (M+H)⁺.

### Example 83K

### (7R,20S)-18-chloro-1-(4-fluorophenyl)-10-({2-[3-(2-methoxyethyl)oxetan-3-yl]pyrimidin-4-yl}methoxy)-19-methyl-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylic acid

Aqueous lithium hydroxide (1 N, 0.7 mL) was added to a mixture of Example 83J (65.6 mg) in ethanol (1.15 mL), tetrahydrofuran (0.35 mL) and methanol (0.35 mL). The reaction mixture was stirred at room temperature for 4 days. The reaction mixture was then quenched with 1N aqueous hydrochloric acid to adjust the pH to 7. The mixture was extracted with 50% methanol/dichloromethane (5 mL×5), and the combined organic layers were concentrated. The residue was purified by reverse-phase HPLC on a Gilson PLC 2020 using a Luna column (250×30 mm, 10 mm) (10-60% acetonitrile/water with 0.1% trifluoroacetic acid) to provide the title compound. ¹H NMR (500 MHz, chloroform-d) δ ppm 8.64 (d, J=5.1 Hz, 1H), 8.61 (d, J=2.2 Hz, 1H), 7.52 (d, J=7.9 Hz, 1H), 7.34 (d, J=7.9 Hz, 1H), 7.17 (dt, J=8.3, 5.6 Hz, 4H), 6.98-6.93 (m, 2H), 6.67 (d, J=8.4 Hz, 1H), 6.37 (s, 1H), 5.10-4.91 (m, 2H), 4.35-4.05 (m, 7H), 4.01-3.95 (m, 2H), 3.89 (q, J=7.8 Hz, 2H), 3.78 (s, 2H), 3.74-3.44 (m, 6H), 3.27 (s, 3H), 3.22-2.90 (m, 6H), 2.79 (s, 3H), 2.63-2.50 (m, 1H), 2.23-2.11 (m, 1H), 1.94 (s, 3H). MS (ESI) m/z 908.3 (M+H)⁺.

### Example 84

### (7R,20S)-10-[(2-{(2S)-1-[(benzyloxy)carbonyl]pyrrolidin-2-yl}pyrimidin-4-yl)methoxy]-18-chloro-1-(4-fluorophenyl)-19-methyl-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylic acid

### Example 84A

### ((benzyloxy)carbonyl)-D-proline

To a mixture of D-proline (25 g) in dichloromethane (500 mL) was added triethylamine (26.4 g) at 0° C. Benzyl carbonochloridate (48.2 g) was added to the reaction. The reaction mixture was stirred at 15° C. for 2 hours. The reaction mixture was quenched by addition of saturated aqueous NH₄Cl (250 mL). The mixture was extracted with dichloromethane (3×250 mL). The combined organic layers were dried over Na₂SO₄ and filtered. The filtrate was concentrated under reduced pressure to give the residue which was purified by column chromatography on silica gel (eluted with ethyl acetate) to provide the title compound. ¹H NMR (400 MHz, CDCl₃) δ ppm 7.39-7.17 (m, 5H), 5.18-5.01 (m, 2H), 4.35-4.24 (m, 1H), 3.64-3.54 (m, 1H), 3.52-3.38 (m, 1H), 2.25-2.09 (m, 1H), 2.08-1.98 (m, 1H), 1.97-1.86 (m, 1H), 1.85-1.74 (m, 1H).

### Example 84B

### benzyl (R)-2-carbamoylpyrrolidine-1-carboxylate

To a mixture of Example 84A (25 g) in tetrahydrofuran (250 mL) was added di(1H-imidazol-1-yl)methanone (48.8 g) at 20° C. and the reaction mixture was stirred for 2 hours. Saturated ammonium hydroxide mixture (200 mL) was added to the reaction mixture dropwise at 0° C. The reaction mixture was extracted with dichloromethane (5×50 mL). The combined organic layers were washed with brine (50 mL), dried over Na₂SO₄ and filtered. The filtrate was concentrated under reduced to give a residue which was purified by column chromatography on silica gel (eluted with dichloromethane methanol=100:1 to 40:1) to provide the title compound. ¹H NMR (400 MHz, CDCl₃) δ ppm 7.33 (br s, 5H), 5.18-5.11 (m, 2H), 4.32 (br s, 1H), 3.61-3.35 (m, 2H), 2.35-1.76 (m, 4H).

### Example 84C

### benzyl (R)-2-(imino(methoxy)methyl)pyrrolidine-1-carboxylate

To a mixture of Example 84B (27 g) in dichloromethane (500 mL) was added trimethyloxonium tetrafluoroborate (24.1 g) at 0° C. and the reaction mixture was stirred at 20° C. for 12 hours. The reaction mixture was quenched by addition of saturated aqueous NaHCO₃ (50 mL). The mixture was extracted with dichloromethane (3×75 mL). The combined organic layers were washed with brine (100 mL) and dried over Na₂SO₄. After filtering, the filtrate was concentrated under reduced pressure to provide the title compound. ¹H NMR (400 MHz, CDCl₃) δ ppm 7.27-7.19 (m, 5H), 5.09-5.00 (m, 2H), 4.21-4.29 (m, 1H), 3.71-3.60 (m, 3H), 3.48-3.32 (m, 2H), 2.14-1.94 (m, 1H), 1.92-1.83 (m, 1H), 1.81-1.65 (m, 2H).

### Example 84D

### benzyl (R)-2-carbamimidoylpyrrolidine-1-carboxylate

To a mixture of Example 84C (18 g) in methanol (300 mL) was added ammonium chloride (4.99 g) at 10° C. and the reaction mixture was stirred at 80° C. for 12 hours. The reaction mixture was concentrated under reduce pressure to give a residue which was dissolved in dichloromethane (50 mL). The material was filtered and the filtrate was acidified to pH 4 by addition of diluted aqueous hydrochloric acid (2 N). The aqueous phase was adjusted to pH 12 and was extracted with dichloromethane (3×100 mL). The combined organic layers were dried over Na₂SO₄ and filtered. The filtrate was concentrated under reduced pressure to provide the title compound. ¹H NMR (400 MHz, CDCl₃) δ ppm 9.08 (br s, 2H), 7.41-7.29 (m, 5H), 6.59 (br s, 1H), 5.16-5.01 (m, 2H), 3.62-3.53 (m, 1H), 3.49-3.31 (m, 2H), 2.43-2.20 (m, 1H), 1.98-1.60 (m, 3H).

### Example 84E

### benzyl 2-(4-(dimethoxymethyl)pyrimidin-2-yl)pyrrolidine-1-carboxylate

To a mixture of Example 84D (28 g) in methanol (200 mL) was added Example 100A (29.4 g) at 15° C. and the reaction mixture was stirred at 80° C. for 12 hours. The reaction mixture was concentrated under reduced pressure to give a residue which was purified by column chromatography on silica gel (eluted with petroleum ether:ethyl acetate=50:1 to 10:1) to provide the title compound. ¹H NMR (400 MHz, CDCl₃) δ ppm 8.59-8.78 (m, 1H), 7.29-7.45 (m, 3H), 7.18 (br d, J=2.20 Hz, 2H), 6.96 (br d, J=3.06 Hz, 1H), 5.10-5.18 (m, 2H), 4.98-5.06 (m, 1H), 4.84-4.93 (m, 1H), 3.61-3.89 (m, 2H), 3.31-3.46 (m, 6H), 2.32-2.55 (m, 1H), 2.01-2.08 (m, 2H), 1.87-1.97 (m, 1H).

### Example 84F

### benzyl (R*)-2-(4-(hydroxymethyl)pyrimidin-2-yl)pyrrolidine-1-carboxylate

To a mixture of Example 84E (18 g) in 1,4-dioxane (250 mL) was added aqueous hydrogen chloride (250 mL, 4 N) at 15° C. and the reaction mixture was stirred at 60° C. for 12 hours. The reaction mixture was cooled to 0° C. and aqueous NaOH (200 mL, 4 N) was added slowly. The mixture was then adjusted to pH 8 by addition of 10% aqueous K₂CO₃. NaBH₄ (3.75 g) was added at 0° C. and the reaction mixture was stirred for 1 hour. The reaction mixture was diluted with water (200 mL) and extracted with ethyl acetate (3×500 mL). The combined organic layers were washed with brine (500 mL) and dried over Na₂SO₄. After filtering, the filtrate was concentrated under reduced pressure to give a racemic mixture. The enantiomers were separated on a Thar SFC80 preparative SFC system using a Chiralpak AD-H 250×30 mm i.d. 5u column with a flow rate of 65 g/minute, a system back pressure of 100 bar, a column temperature of 40° C., and a mobile phase of 35% methanol (0.1% NH₃H₂O) in CO₂ to provide the title compound. ¹H NMR (400 MHz, dimethyl sulfoxide) δ ppm 8.66 (d, J=5.3 Hz, 1H), 8.23 (s, 1H), 7.38 (d, J=4.8 Hz, 1H), 7.25 (br s, 4H), 5.32 (t, J=5.7 Hz, 1H), 5.00-4.91 (m, 2H), 4.50 (br d, J=5.3 Hz, 2H), 3.69-3.52 (m, 2H), 2.42-2.31 (m, 1H), 2.00-1.83 (m, 3H). LC/MS (ESI) m/z 314 (M+H)⁺.

### Example 84G

### benzyl (S*)-2-(4-(hydroxymethyl)pyrimidin-2-yl)pyrrolidine-1-carboxylate

The title compound was also isolated during the synthesis of Example 84F. ¹H NMR (400 MHz, dimethyl sulfoxide) δ ppm 8.66 (d, J=5.3 Hz, 1H), 8.23 (s, 1H), 7.38 (d, J=5.3 Hz, 1H), 7.35-6.74 (m, 4H), 5.32 (t, J=5.5 Hz, 1H), 5.00-4.91 (m, 2H), 4.50 (br d, J=4.4 Hz, 2H), 3.68-3.51 (m, 2H), 2.42-2.31 (m, 1H), 2.02-1.81 (m, 3H). LC/MS (ESI) m/z 314 (M+H)⁺.

### Example 84H

### benzyl (S*)-2-(4-(chloromethyl)pyrimidin-2-yl)pyrrolidine-1-carboxylate

To a mixture of Example 84G (500 mg) in anhydrous CH₂Cl₂ (10 mL) was added triphenylphosphine (544 mg) at 0° C. The mixture was stirred at 0° C. for 45 minutes, and N-chlorosuccinimide (234 mg) was added. The reaction mixture was allowed to warm to room temperature overnight, and was directly loaded onto a silica gel column that was eluted with 20-60% ethyl acetate in heptane to provide the title compound. The material was used immediately in the next step.

### Example 84I

### ethyl (7R,20S)-10-[(2-{(2S*)-1-[(benzyloxy)carbonyl]pyrrolidin-2-yl}pyrimidin-4-yl)methoxy]-18-chloro-1-(4-fluorophenyl)-19-methyl-15-[2-(4-methylpiperazin-1-yl)ethyl]-7,8,15,16-tetrahydro-14H-17,20-etheno-9,13-(metheno)-6-oxa-2-thia-3,5,15-triazacyclooctadeca[1,2,3-cd]indene-7-carboxylate

A mixture of Example 65M (79 mg), Example 84H (71.8), and cesium carbonate (141 mg) in anhydrous N,N-dimethylformamide (5 mL) was stirred at room temperature overnight. The reaction mixture was partitioned between ethyl acetate and brine. The organic phase was washed with brine, and concentrated. The residue was separated by flash chromatography (0-20% methanol containing 3% NH₄OH in CH₂Cl₂) to provide the title compound. MS (ESI) m/z 1025 (M+H)⁺.

### Example 84J

### (7R,20S)-10-[(2-{(2S*)-1-[(benzyloxy)carbonyl]pyrrolidin-2-yl}pyrimidin-4-yl)methoxy]-18-chloro-1-(4-fluorophenyl)-19-methyl-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylic acid

To a mixture of Example 84I (90 mg) in tetrahydrofuran (1.5 mL) was added a mixture of lithium hydroxide monohydrate (30 mg) in water (1.5 mL) and methanol (1.5 mL). The mixture was stirred at room temperature for 1 day before trifluoroacetic acid (0.2 mL) was added. The mixture was concentrated. The residue was separated by HPLC (Zorbax, C-18, 250×5.0 column, mobile phase A: 0.1% trifluoroacetic acid in H₂O; B: 0.1% trifluoroacetic acid in CH₃CN; 0-70% gradient). The desired fraction was lyophilized to provide the title compound. ¹H NMR (400 MHz, dimethyl sulfoxide-d₆) δ ppm 8.99 (d, J=5.0 Hz, 1H), 8.93 (d, J=5.0 Hz, 1H), 8.67 (d, J=4.5 Hz, 2H), 8.60-8.56 (m, 1H), 8.53 (d, J=5.1 Hz, 1H), 8.47 (dd, J=11.4, 5.1 Hz, 1H), 7.83 (d, J=5.0 Hz, 1H), 7.79 (d, J=5.0 Hz, 1H), 7.54 (dd, J=8.1, 3.5 Hz, 2H), 7.40-7.28 (m, 4H), 7.28-7.22 (m, 2H), 7.21-7.07 (m, 4H), 6.87-6.77 (m, 3H), 6.65 (s, 1H), 6.52-6.45 (m, 2H), 6.01-5.93 (m, 2H), 5.18-4.87 (m, 5H), 4.75 (dd, J=12.9, 6.1 Hz, 2H), 4.51-4.30 (m, 2H), 4.22 (s, 2H), 3.26-2.93 (m, 4H), 2.81 (d, J=3.6 Hz, 3H), 2.44-2.31 (m, 1H), 1.96-1.81 (m, 2H), 1.75 (d, J=4.2 Hz, 3H). MS (ESI) m/z 997 (M+H)⁺.

### Example 85

### (7R,20S)-18-chloro-1-(4-fluorophenyl)-19-methyl-15-[2-(4-methylpiperazin-1-yl)ethyl]-10-({2-[(2R)-oxolan-2-yl]pyrimidin-4-yl}methoxy)-7,8,15,16-tetrahydro-14H-17,20-etheno-13,9-(metheno)-6-oxa-2-thia-3,5,15-triazacyclooctadeca[1,2,3-cd]indene-7-carboxylic acid

### Example 85A

### tetrahydrofuran-2-carboxamide

To a mixture of tetrahydrofuran-2-carboxylic acid (12 g) in tetrahydrofuran (200 mL) was added di(1H-imidazol-1-yl) methanone (53.3 g) at 15° C. and the reaction was mixture was stirred for 2 hours. Ammonium hydroxide (100 mL) was added to the reaction at 0° C. and the reaction mixture was stirred at 15° C. for 2 hours. The reaction mixture was separated and the aqueous phase was extracted with dichloromethane (5×50 mL). The combined organic layers were dried over Na₂SO₄ and filtered. The filtrate was concentrated to give the residue which was purified by column chromatography on silica gel (eluted with dichloromethane:methane=200:1 to 30:1) to provide the title compound. ¹H NMR (400 MHz, CDCl₃) δ ppm 1.86-1.95 (m, 2H), 2.08 (td, J=13.37, 6.14 Hz, 1H), 2.23-2.34 (m, 1H), 3.85-4.00 (m, 2H), 4.35 (dd, J=8.55, 5.92 Hz, 1H), 5.97 (br s, 1H), 6.61 (br s, 1H).

### Example 85B

### methyl tetrahydrofuran-2-carbimidate

To a mixture of Example 85A (16 g) in dichloromethane (200 mL) was added trimethyloxonium tetrafluoroborate (22.6 g) at 0° C. The reaction mixture was stirred at 15° C. for 12 hours. The reaction mixture was quenched by addition of saturated aqueous NaHCO₃ (1 L) and was extracted with ethyl acetate (3×100 mL). The combined organic layers were dried over Na₂SO₄. After filtering, the filtrate was concentrated to provide the title compound. ¹H NMR (400 MHz, CDCl₃) δ ppm 1.17-1.29 (m, 1H), 1.78-2.05 (m, 3H), 2.12-2.28 (m, 1H), 3.69-3.77 (m, 3H), 3.81-4.01 (m, 1H), 3.81-4.01 (m, 1H), 3.83-4.02 (m, 1H), 4.22-4.30 (m, 1H), 4.44 (dd, J=8.31, 5.26 Hz, 1H), 4.99-5.23 (m, 1H), 4.99-5.23 (m, 1H), 5.05 (s, 1H), 7.59 (br s, 1H).

### Example 85C

### tetrahydrofuran-2-carboximidamide

To a mixture of Example 85B (24.5 g) in methanol (100 mL) was added ammonium chloride (15.2 g) at 10° C. The reaction mixture was stirred at 70° C. for 12 hours. The reaction mixture was concentrated to give a residue which was diluted with dichloromethane (50 mL) and was filtered. The filtrate was concentrated to provide the title compound. ¹H NMR (400 MHz, dimethyl sulfoxide-d₆) δ ppm 1.75-1.93 (m, 3H), 2.07-2.45 (m, 1H), 2.10-2.20 (m, 1H), 3.40 (s, 1H), 3.62 (s, 1H), 3.73-3.83 (m, 1H), 3.93-4.02 (m, 1H), 4.59 (br s, 1H), 4.39 (dd, J=8.38, 4.85 Hz, 1H), 4.59-4.66 (m, 1H), 9.01 (br s, 2H).

### Example 85D

### 4-(dimethoxymethyl)-2-(tetrahydrofuran-2-yl)pyrimidine

To a mixture of Example 85C (20 g) in methanol (1 L) was added sodium methanolate (105 mL) at 0° C. (E)-4-(Dimethylamino)-1,1-dimethoxybut-3-en-2-one (50.6 g) was added to the reaction. The reaction mixture was stirred at 70° C. for 12 hours. The reaction mixture was quenched by the addition of saturated aqueous NH₄Cl mixture (500 mL) and was extracted with ethyl acetate (3×500 mL). The combined organic layers were washed with brine (1 L), dried over Na₂SO₄ and filtered. The filtrate was concentrated under reduced pressure, and the crude material was purified by column chromatography on silica gel (eluted with petroleum ether:ethyl acetate=50:1 to 10:1) to provide the title compound. ¹H NMR (400 MHz, CDCl₃) δ ppm 1.99-2.16 (m, 3H), 2.39-2.48 (m, 1H), 3.43 (d, J=8.60 Hz, 6H), 3.99-4.07 (m, 1H), 4.23 (q, J=6.61 Hz, 1H), 5.15 (br t, J=6.61 Hz, 1H), 5.29 (s, 1H), 7.43 (br d, J=4.63 Hz, 1H), 8.80 (br s, 1H).

### Example 85E

### (R*)-(2-(tetrahydrofuran-2-yl)pyrimidin-4-yl)methanol

To a mixture of Example 85D (3.5 g) in 1,4-dioxane (70 mL) was added 4 M aqueous hydrogen chloride (70 mL) at 15° C. and the reaction mixture was stirred at 60° C. for 12 hours. The reaction mixture was cooled to 0° C. and the pH was adjusted to approximately seven by progressively adding saturated aqueous NaOH. NaBH₄ (1.18 g) was added at 0° C. and the reaction mixture was stirred for 1 hour. The reaction mixture was diluted with water (250 mL) and was extracted with dichloromethane (10×50 mL). The combined organic layers were dried over Na₂SO₄ and filtered. The filtrate was concentrated and the crude material was purified by column chromatography on silica gel (eluted with dichloromethane:methane=50:1 to 10:1) to provide the title compound. The enantiomers were separated on a Thar SFC80 preparative SFC system using a Chiralpak AD-H 250×30 mm i.d. 5 μm column with a flow rate of 46 g/minute, a system back pressure of 100 bar, a column temperature of 40° C., and a mobile phase of 13% methanol (0.1% NH₃H₂O) in CO₂ to provide the title compound. ¹H NMR (400 MHz, CDCl₃) δ ppm 1.99-2.18 (m, 3H), 2.38-2.49 (m, 1H), 4.03 (td, J=7.70, 5.62 Hz, 1H), 4.17-4.24 (m, 1H), 4.77 (s, 2H), 5.12 (dd, J=7.46, 5.99 Hz, 1H), 7.20 (d, J=5.14 Hz, 1H), 8.69 (d, J=5.13 Hz, 1H).

### Example 85F

### (S*)-(2-(tetrahydrofuran-2-yl)pyrimidin-4-yl)methanol

The title compound was isolated during the synthesis of Example 85E. ¹H NMR (400 MHz, CDCl₃) δ ppm 1.97-2.19 (m, 3H), 2.34-2.50 (m, 1H), 3.56 (br s, 1H), 4.01-4.05 (m, 1H), 4.17-4.20 (m, 1H), 4.76 (s, 2H), 5.11 (dd, J=7.52, 6.05 Hz, 1H), 7.21 (d, J=5.14 Hz, 1H), 8.68 (d, J=5.14 Hz, 1H). LC/MS (ESI) m/z 181 (M+H)⁺.

### Example 85G

### (7R,20S)-18-chloro-1-(4-fluorophenyl)-19-methyl-15-[2-(4-methylpiperazin-1-yl)ethyl]-10-({2-[(2R*)-oxolan-2-yl]pyrimidin-4-yl}methoxy)-7,8,15,16-tetrahydro-14H-17,20-etheno-13,9-(metheno)-6-oxa-2-thia-3,5,15-triazacyclooctadeca[1,2,3-cd]indene-7-carboxylic acid

The title compound was prepared according to the protocols for Example 84H-J, substituting Example 85E for Example 84G. ¹H NMR (400 MHz, dimethyl sulfoxide-d₆) δ ppm 8.66 (s, 1H), 8.61-8.55 (m, 1H), 7.52 (d, J=8.0 Hz, 2H), 7.30 (d, J=7.9 Hz, 2H), 7.28-7.10 (m, 6H), 6.84 (t, J=9.1 Hz, 1H), 6.48 (s, 1H), 5.92 (dd, J=8.4, 4.7 Hz, 1H), 5.20-4.98 (m, 4H), 4.89 (dt, J=7.9, 5.7 Hz, 2H), 4.37 (q, J=14.0 Hz, 2H), 4.19 (s, 2H), 4.03-3.91 (m, 2H), 3.84 (td, J=7.6, 5.3 Hz, 2H), 3.23-2.94 (m, 4H), 2.81 (s, 3H), 2.24 (tdd, J=10.0, 5.0, 2.7 Hz, 2H), 2.07-1.82 (m, 4H), 1.74 (s, 3H). MS (ESI) m/z 864 (M+H)⁺.

### Example 86

### (7R,20S)-18-chloro-1-(4-fluorophenyl)-19-methyl-15-[2-(4-methylpiperazin-1-yl)ethyl]-10-({2-[(2S*)-oxolan-2-yl]pyrimidin-4-yl}methoxy)-7,8,15,16-tetrahydro-14H-17,20-etheno-13,9-(metheno)-6-oxa-2-thia-3,5,15-triazacyclooctadeca[1,2,3-cd]indene-7-carboxylic acid

The title compound was prepared according to the protocols for Example 84H-J, substituting Example 85F for Example 84G. ¹H NMR (400 MHz, dimethyl sulfoxide-d₆) δ ppm 8.66 (s, 1H), 8.58 (d, J=5.2 Hz, 1H), 7.52 (d, J=7.9 Hz, 1H), 7.31 (t, J=7.4 Hz, 1H), 7.27-7.11 (m, 6H), 6.81 (d, J=8.5 Hz, 1H), 6.48 (d, J=2.2 Hz, 1H), 5.94 (dd, J=8.8, 4.5 Hz, 1H), 5.20-4.99 (m, 4H), 4.88 (dd, J=7.6, 5.4 Hz, 2H), 4.35 (s, 2H), 4.17 (s, 2H), 3.97 (q, J=7.0 Hz, 2H), 3.84 (td, J=7.7, 5.1 Hz, 2H), 3.27-2.96 (m, 6H), 2.80 (s, 3H), 2.26 (tdd, J=10.4, 5.3, 2.7 Hz, 2H), 2.13-1.87 (m, 4H), 1.73 (s, 3H). MS (ESI) m/z 864 (M+H)⁺.

### Example 87

### (7R,20S)-18-chloro-1-(4-fluorophenyl)-19-methyl-15-[2-(4-methylpiperazin-1-yl)ethyl]-10-({2-[(2S*)-pyrrolidin-2-yl]pyrimidin-4-yl}methoxy)-7,8,15,16-tetrahydro-14H-17,20-etheno-13,9-(metheno)-6-oxa-2-thia-3,5,15-triazacyclooctadeca[1,2,3-cd]indene-7-carboxylic acid

A mixture of Example 84J (32 mg) was dissolved in methanol (10 mL). The mixture was purged with nitrogen and 20 mg of palladium on carbon (10%) was added. The reaction mixture was purged with hydrogen and was stirred at room temperature overnight. The material was filtered off. The filtrate was concentrated and the residue was purified by HPLC (Zorbax, C-18, 250×5.0 column, mobile phase A: 0.1% trifluoroacetic acid in H₂O; B: 0.1% trifluoroacetic acid in CH₃CN; 0-70% gradient. The desired fraction was lyophilized to provide the title compound. ¹H NMR (400 MHz, dimethyl sulfoxide-d₆) δ ppm 9.81 (s, 1H), 8.96 (s, OH), 8.73 (d, J=5.2 Hz, 1H), 8.65 (s, 1H), 7.50 (d, J=7.9 Hz, 1H), 7.35 (d, J=5.2 Hz, 1H), 7.31-7.20 (m, 3H), 7.20-7.11 (m, 3H), 6.78 (d, J=8.4 Hz, 1H), 6.52 (d, J=2.2 Hz, 1H), 5.95 (dd, J=9.2, 4.3 Hz, 1H), 5.16 (d, J=15.2 Hz, 2H), 5.04 (d, J=15.3 Hz, 2H), 4.88 (s, 2H), 4.21 (s, 3H), 4.04 (s, 3H), 3.25-2.96 (m, 8H), 2.78 (s, 3H), 2.13-1.94 (m, 4H), 1.72 (s, 3H), 1.23 (s, 2H). MS (ESI) m/z 864 (M+H)⁺.

### Example 88

### (7R,16R)-19,23-dichloro-1-(4-fluorophenyl)-10-{[2-(2-methoxyphenyl)pyrimidin-4-yl]methoxy}-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic acid

### Example 88A

### (R)-3-(bis(4-methoxyphenyl)(phenyl)methoxy)-2-(4-bromo-2,6-dichlorophenoxy)propyl 4-methylbenzenesulfonate

To a mixture of Example 73B (300 mg) and 4-bromo-2,6-dichlorophenol (172 mg) in tetrahydrofuran (5.5 mL) was added triphenylphosphine (215 mg) and di-tert-butyl azodicarboxylate (189 mg). The reaction mixture was heated to 45° C. After 2.5 hours, more triphenylphosphine (72 mg) and di-tert-butyl azodicarboxylate (63 mg) were added, and the reaction mixture was heated for another hour. The reaction mixture was cooled and was concentrated. The crude residue was purified by normal phase MPLC on a Teledyne Isco Combiflash Rf+ (5-45% ethyl acetate in heptanes) to provide the title compound which was contaminated with some tert-butyl 2-(tert-butoxy)hydrazinecarboxylate. ¹H NMR (400 MHz, CDCl₃) δ ppm 7.71 (d, 2H), 7.39-7.12 (m, 13H), 6.86-6.73 (m, 4H), 4.51-4.29 (m, 3H), 3.80 (s, 6H), 3.52-3.35 (m, 2H), 2.43 (s, 3H).

### Example 88B

### (R)-3-(bis(4-methoxyphenyl)(phenyl)methoxy)-2-(2,6-dichloro-4-(4,4,5,5-tetramethyl-1,3,2-dioxaborolan-2-yl)phenoxy)propyl 4-methylbenzenesulfonate

To a vial containing potassium acetate (97 mg, heated at 100° C. under vacuum for at least one hour), 1,1′-bis(diphenylphosphino)ferrocene-palladium(II)dichloride dichloromethane complex (20.14 mg), and bis(pinacolato)diboron (150 mg) was added a 2-methyl tetrahydrofuran (2.5 mL) and (R)-3-(bis(4-methoxyphenyl)(phenyl)methoxy)-2-(4-bromo-2,6-dichlorophenoxy)propyl 4-methylbenzenesulfonate (381 mg). The mixture was purged with nitrogen and was heated at 90° C. overnight. The reaction mixture was cooled, diluted with ethyl acetate, filtered over diatomaceous earth and concentrated. The crude residue was purified by normal phase MPLC on a Teledyne Isco Combiflash Rf+ (0-25% ethyl acetate in heptanes) to provide the title compound. ¹H NMR (400 MHz, CDCl₃) δ ppm 7.70 (d, 2H), 7.62 (s, 2H), 7.33-7.13 (m, 11H), 6.83-6.71 (m, 4H), 4.52-4.30 (m, 3H), 3.79 (s, 6H), 3.53-3.37 (m, 2H), 2.42 (s, 3H), 1.35 (s, 12H).

### Example 88C

### (R)-ethyl 2-((5-(4-(((R)-1-(bis(4-methoxyphenyl)(phenyl)methoxy)-3-(tosyloxy)propan-2-yl)oxy)-3,5-dichlorophenyl)-6-(4-fluorophenyl)thieno[2,3-d]pyrimidin-4-yl)oxy)-3-(5-((tert-butyldimethylsilyl)oxy)-2-((2-(2-methoxyphenyl)pyrimidin-4-yl)methoxy)phenyl)propanoate

A vial containing Example 88B (233 mg), Example 68C (185 mg), cesium carbonate (214 mg) and bis(di-tert-butyl(4-dimethylaminophenyl)phosphine)dichloropalladium(II) (15.49 mg) was evacuated and backfilled with nitrogen several times. To the vial was added degassed tetrahydrofuran (1.8 mL) and water (440 μL), and the reaction mixture was stirred overnight at room temperature. 1-Pyrrolidinecarbodithioic acid ammonium salt (3.59 mg) was added, and the reaction was allowed to stir for 30 minutes. The reaction mixture was diluted with ethyl acetate and was filtered over diatomaceous earth. Brine and water were added, and the aqueous layer was extracted with ethyl acetate three times. The combined organic layers were dried over anhydrous sodium sulfate, filtered and concentrated. The crude residue was purified by normal phase MPLC on a Teledyne Isco Combiflash Rf+ (5-65% ethyl acetate in heptanes) to provide the title compound. MS (ESI) m/z 1456.4 (M+H)⁺.

### Example 88D

### (R)-ethyl 2-((5-(4-(((R)-1-(bis(4-methoxyphenyl)(phenyl)methoxy)-3-(tosyloxy)propan-2-yl)oxy)-3,5-dichlorophenyl)-6-(4-fluorophenyl)thieno[2,3-d]pyrimidin-4-yl)oxy)-3-(5-hydroxy-2-((2-(2-methoxyphenyl)pyrimidin-4-yl)methoxy)phenyl)propanoate

To a mixture of Example 88C (263 mg) in tetrahydrofuran (1.8 mL) was added tetrabutylammonium fluoride (180 μL, 1 M in tetrahydrofuran), and the reaction mixture was allowed to stir. After 25 minutes, the reaction mixture was quenched with saturated aqueous ammonium chloride and was extracted with ethyl acetate three times. The combined organic layers were dried over anhydrous sodium sulfate, filtered and concentrated. The crude residue was purified by normal phase MPLC on a Teledyne Isco Combiflash Rf+ (10-75% ethyl acetate in heptanes) to provide the title compound. MS (ESI) m/z 1344.6 (M+H)⁺.

### Example 88E

### ethyl (7R,16S)-16-{[bis(4-methoxyphenyl)(phenyl)methoxy]methyl}-19,23-dichloro-1-(4-fluorophenyl)-10-{[2-(2-methoxyphenyl)pyrimidin-4-yl]methoxy}-7,8,15,16-tetrahydro-18,21-etheno-9,13-(metheno)-6,14,17-trioxa-2-thia-3,5-diazacyclononadeca[1,2,3-cd]indene-7-carboxylate

A mixture of Example 88D (200 mg) and cesium carbonate (485 mg) in tetrahydrofuran (18 mL) was heated at 65° C. overnight. The reaction mixture was cooled and transferred to a separatory funnel with water and ethyl acetate. The aqueous layer was extracted with ethyl acetate three times. The combined organic layers were dried over anhydrous sodium sulfate, filtered and concentrated. The crude residue was purified by normal phase MPLC on a Teledyne Isco Combiflash Rf+ (15-90% ethyl acetate in heptanes) to provide the title compound which was carried forward without further purification. MS (ESI) m/z 1171.3 (M+H)⁺.

### Example 88F

### ethyl (7R,16R)-19,23-dichloro-1-(4-fluorophenyl)-16-(hydroxymethyl)-10-{[2-(2-methoxyphenyl)pyrimidin-4-yl]methoxy}-7,8,15,16-tetrahydro-18,21-etheno-9,13-(metheno)-6,14,17-trioxa-2-thia-3,5-diazacyclononadeca[1,2,3-cd]indene-7-carboxylate

To a mixture of Example 88E (152 mg) in dichloromethane (650 μL) and methanol (650 μL) was added formic acid (647 μL), and the reaction mixture was allowed to stir. After 30 minutes, the reaction mixture was quenched slowly with saturated aqueous sodium bicarbonate and was extracted with ethyl acetate three times. The combined organics extracts were dried over anhydrous sodium sulfate, filtered and concentrated. The residue was purified by normal phase MPLC on a Teledyne Isco Combiflash Rf+ (30-100% ethyl acetate in heptanes) and the desired product containing fractions were concentrated and repurified by RP-HPLC on a Gilson PLC 2020 using a Luna column (250×50 mm, 10 mm) (20-100% over 30 minutes with acetonitrile in water containing 0.1% trifluoroacetic acid). Product containing fractions were neutralized with saturated aqueous sodium bicarbonate and were extracted with dichloromethane three times. The combined organic extracts were dried over anhydrous sodium sulfate, filtered and concentrated to give to provide the title compound. MS (ESI) m/z 869.0 (M+H)⁺.

### Example 88G

### ethyl (7R,16S)-19,23-dichloro-1-(4-fluorophenyl)-10-{[2-(2-methoxyphenyl)pyrimidin-4-yl]methoxy}-16-{[(4-methylbenzene-1-sulfonyl)oxy]methyl}-7,8,15,16-tetrahydro-18,21-etheno-9,13-(metheno)-6,14,17-trioxa-2-thia-3,5-diazacyclononadeca[1,2,3-cd]indene-7-carboxylate

To a mixture of Example 88F (79 mg) and triethylamine (38.0 μL) in dichloromethane (900 μL) was added p-toluenesulfonyl chloride (34.6 mg), and the reaction mixture was allowed to stir. After 4 hours, additional p-toluenesulfonyl chloride (5.8 mg) was added, and the reaction mixture was allowed to stir for another hour. The reaction mixture was diluted with dichloromethane and water. The aqueous layer was extracted with dichloromethane three times, and the combined organic layers were dried over anhydrous sodium sulfate, filtered and concentrated. The crude residue was purified by normal phase MPLC on a Teledyne Isco Combiflash Rf+ (20-80% ethyl acetate in heptanes) to provide the title compound. MS (ESI) m/z 1023.2 (M+H)⁺.

### Example 88H

### ethyl (7R,16R)-19,23-dichloro-1-(4-fluorophenyl)-10-{[2-(2-methoxyphenyl)pyrimidin-4-yl]methoxy}-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylate

A mixture of Example 88G (75 mg) and 1-methylpiperazine (243 μL) in dimethyl formamide (240 μL) was warmed at 45° C. overnight. The reaction mixture was cooled, taken up in dimethyl sulfoxide (600 μL) and purified by RP-HPLC on a Gilson PLC 2020 using a Luna column (250×50 mm, 10 mm) (5-85% over 30 minutes with acetonitrile in water containing 0.1% trifluoroacetic acid) to provide the title compound after lyophilyzation. MS (ESI) m/z 951.4 (M+H)⁺.

### Example 88I

### (7R,16R)-19,23-dichloro-1-(4-fluorophenyl)-10-{[2-(2-methoxyphenyl)pyrimidin-4-yl]methoxy}-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic acid

To a mixture of Example 88H (26.4 mg) in tetrahydrofuran (310 μL) and methanol (310 μL) at 0° C. was added a mixture of lithium hydroxide (13.40 mg) in water (310 μL), and the reaction mixture was allowed to stand at 0° C. overnight. The reaction mixture was quenched with trifluoreacetic acid (51.7 μL), taken up in dimethyl sulfoxide and purified by RP-HPLC on a Gilson PLC 2020 using a Luna column (250×50 mm, 10 mm) (5-65% over 45 minutes with acetonitrile in water containing 0.1% trifluoroacetic acid) to provide the title compound after lyophilyzation. ¹H NMR (500 MHz, dimethyl sulfoxide-d₆) δ 8.90 (d, 1H), 8.75 (s, 1H), 7.58 (d, 1H), 7.54 (dd, 1H), 7.50-7.43 (m, 2H), 7.41 (d, 1H), 7.32-7.20 (m, 4H), 7.15 (d, 1H), 7.09-7.02 (m, 1H), 6.92 (d, 1H), 6.81 (dd, 1H), 6.31 (dd, 1H), 5.96 (d, 1H), 5.25-5.10 (m, 2H), 5.01-4.91 (m, 1H), 4.41-4.31 (m, 2H), 3.76 (s, 3H), 3.73 (d, 1H), 3.48-3.15 (m, 4H), 3.14-2.95 (m, 4H), 2.92-2.74 (m, 5H). MS (ESI) m/z 923.3 (M+H)⁺.

### Example 89

### (7R,20S)-18-chloro-1-(4-fluorophenyl)-19-methyl-15-[2-(4-methylpiperazin-1-yl)ethyl]-10-({2-[(1S,4S)-2-oxa-5-azabicyclo[2.2.1]heptan-5-yl]pyrimidin-4-yl}methoxy)-7,8,15,16-tetrahydro-14H-17,20-etheno-13,9-(metheno)-6-oxa-2-thia-3,5,15-triazacyclooctadeca[1,2,3-cd]indene-7-carboxylic acid

### Example 89A

### methyl 2-((1S,4S)-2-oxa-5-azabicyclo[2.2.1]heptan-5-yl)pyrimidine-4-carboxylate

Methyl 2-chloropyrimidine-4-carboxylate (2.4 g) and (1S,4S)-2-oxa-5-azabicyclo[2.2.1]heptane hydrochloride (2.0 g) were dissolved in dioxane (20 mL). Trimethylamine (4.0 mL) was added and the reaction was stirred at 50° C. under nitrogen overnight. The reaction mixture was partitioned between water and ethyl acetate. The organic layer was washed with brine, and dried over sodium sulfate. After filtration, the crude residue was purified by silica gel chromatography, eluting with 30/70 heptanes/ethyl acetate, to provide the title compound. MS (DCI) m/z 235.9 (M+H)⁺.

### Example 89B

### (2-((1S,4S)-2-oxa-5-azabicyclo[2.2.1]heptan-5-yl)pyrimidin-4-yl)methanol

Example 89A was dissolved in methanol (48 mL) under nitrogen, cooled to −13° C., and sodium borohydride (1.6 g) was added in four portions over 10 minutes. The reaction mixture was stirred at −13° C. for 2.5 hours, and saturated aqueous ammonium chloride (25 mL) was carefully added. The reaction mixture was stirred for 5 minutes. The reaction mixture was partitioned between water and ethyl acetate. The organic layer was washed with brine. The combined aqueous layers were extracted with ethyl acetate, dried sodium sulfate, and filtered. The crude residue was purified by silica gel chromatography, eluting with 97.5/2.5 ethyl acetate/methanol, to provide the title compound. MS (DCI) m/z 208.0 (M+H)⁺.

### Example 89C

### (2-((1S,4S)-2-oxa-5-azabicyclo[2.2.1]heptan-5-yl)pyrimidin-4-yl)methyl methanesulfonate

Example 89B (104 mg) was dissolved in dichloromethane (2.5 mL). Triethylamine (0.092 mL) was added, and the reaction mixture was cooled to 0° C. Methanesulfonyl chloride (0.051 mL) was added. The reaction mixture was stirred cold for 5 minutes, the bath was removed, and the reaction was stirred at room temperature for 75 minutes. The reaction mixture was partitioned between saturated aqueous sodium bicarbonate and dichloromethane. The organic layer was washed with brine. The combined aqueous layers were extracted with ethyl acetate, and the combined organic layers were dried over sodium sulfate. The crude product was carried on with no further purification.

### Example 89D

### ethyl (7R,20S)-18-chloro-1-(4-fluorophenyl)-19-methyl-15-[2-(4-methylpiperazin-1-yl)ethyl]-10-({2-[(1S,4S)-2-oxa-5-azabicyclo[2.2.1]heptan-5-yl]pyrimidin-4-yl}methoxy)-7,8,15,16-tetrahydro-14H-17,20-etheno-13,9-(metheno)-6-oxa-2-thia-3,5,15-triazacyclooctadeca[1,2,3-cd]indene-7-carboxylate

The title compound was prepared by substituting Example 89C for Example 65E in Example 65N. MS (ESI) m/z 919.5 (M+H)⁺.

### Example 89E

### (7R,20S)-18-chloro-1-(4-fluorophenyl)-19-methyl-15-[2-(4-methylpiperazin-1-yl)ethyl]-10-({2-[(1S,4S)-2-oxa-5-azabicyclo[2.2.1]heptan-5-yl]pyrimidin-4-yl}methoxy)-7,8,15,16-tetrahydro-14H-17,20-etheno-13,9-(metheno)-6-oxa-2-thia-3,5,15-triazacyclooctadeca[1,2,3-cd]indene-7-carboxylic acid

The title compound was prepared by substituting Example 89D for Example 65N in Example 65O. ¹H NMR (500 MHz, dimethylsulfoxide-d₆) δ ppm 8.57 (s, 1H), 7.91 (d, 1H), 7.38 (d, 1H), 7.24 (d, 1H), 7.15 (m, 2H), 7.07 (m, 2H), 6.90 (d, 1H), 6.59 (s, 1H), 6.52 (d, 1H), 6.31 (d, 1H), 5.84 (m, 1H), 4.84 (br d, 3H), 4.69 (d, 1H), 4.62 (d, 1H), 3.76 (m, 2H), 3.64 (m, 4H), 3.47 (m, 4H), 3.40 (m, 4H), 3.33 (m, 2H), 2.97 (m, 1H), 2.88 (m, 2H), 2.61 (m, 2H), 2.26 (s, 3H), 1.84 (m, 2H), 1.54 (s, 3H). MS (ESI) m/z 891.3 (M+H)⁺.

### Example 90

### (7R,20S)-18-chloro-1-(4-fluorophenyl)-19-methyl-10-({2-[(3R)-3-methylmorpholin-4-yl]pyrimidin-4-yl}methoxy)-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylic acid

### Example 90A

### (R)-methyl 2-(3-methylmorpholino)pyrimidine-4-carboxylate

The title compound was prepared by substituting (R)-3-methylmorpholine for (1S,4S)-2-oxa-5-azabicyclo[2.2.1]heptane in Example 89A. MS (DCI) m/z 238.0 (M+H)⁺.

### Example 90B

### (R)-(2-(3-methylmorpholino)pyrimidin-4-yl)methanol

The title compound was prepared by substituting Example 90A for Example 89A in Example 89B. MS (DCI) m/z 210.0 (M+H)⁺.

### Example 90C

### (R)-(2-(3-methylmorpholino)pyrimidin-4-yl)methyl methanesulfonate

The title compound was prepared by substituting Example 90B for Example 89B in Example 89C. MS (DCI) m/z 287.9 (M+H)⁺.

### Example 90D

### ethyl (7R,20S)-18-chloro-1-(4-fluorophenyl)-19-methyl-10-({2-[(3R)-3-methylmorpholin-4-yl]pyrimidin-4-yl}methoxy)-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylate

The title compound was prepared by substituting Example 90C for Example 65E in Example 65N. MS (ESI) m/z 921.2 (M+H)⁺.

### Example 90E

### (7R,20S)-18-chloro-1-(4-fluorophenyl)-19-methyl-10-({2-[(3R)-3-methylmorpholin-4-yl]pyrimidin-4-yl}methoxy)-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylic acid

The title compound was prepared by substituting Example 90D for Example 65N in Example 65O. ¹H NMR (400 MHz, dimethylsulfoxide-d₆) δ ppm 8.65 (s, 1H), 8.09 (d, 1H), 7.54 (d, 1H), 7.32 (d, 1H), 7.23, (m, 3H), 7.14 (m, 2H), 6.81 (d, 1H), 6.54 (s, 1H), 6.37 (d, 1H), 5.93 (dd, 1H), 4.97 (d, 1H), 4.82 (d, 1H), 4.55 (m, 2H), 4.49 (d, 1H), 4.39 (d, 1H), 4.25 (s, 1H), 4.19 (d, 2H), 3.91 (m, 1H), 3.70 (d, 2H), 3.57 (m, 6H), 3.40 (m, 4H), 3.21 (m, 1H), 3.10 (m, 4H), 2.82 (s, 3H), 1.70 (s, 3H), 1.16 (d, 3H). MS (ESI) m/z 893.4 (M+H)⁺.

### Example 91

### (7R,16R,21S)-19-chloro-1-(4-fluorophenyl)-16-{[(2-methoxyethyl)(methyl)amino]methyl}-10-{[2-(2-methoxyphenyl)pyrimidin-4-yl]methoxy}-20-methyl-7,8,15,16-tetrahydro-18,21-etheno-13,9-(metheno)-6,14,17-trioxa-2-thia-3,5-diazacyclononadeca[1,2,3-cd]indene-7-carboxylic acid

### Example 91A

### ethyl (7R,16R,21S)-19-chloro-1-(4-fluorophenyl)-16-{[(2-methoxyethyl)(methyl)amino]methyl}-10-{[2-(2-methoxyphenyl)pyrimidin-4-yl]methoxy}-20-methyl-7,8,15,16-tetrahydro-18,21-etheno-9,13-(metheno)-6,14,17-trioxa-2-thia-3,5-diazacyclononadeca[1,2,3-cd]indene-7-carboxylate

Example 91A was prepared according to the procedure described for Example 73J, substituting 2-methoxy-N-methylethanamine for 1-methylpiperazine. LC/MS (APCI) m/z 920.2 (M+H)⁺.

### Example 91B

### (7R,16R,21S)-19-chloro-1-(4-fluorophenyl)-16-{[(2-methoxyethyl)(methyl)amino]methyl}-10-{[2-(2-methoxyphenyl)pyrimidin-4-yl]methoxy}-20-methyl-7,8,15,16-tetrahydro-18,21-etheno-13,9-(metheno)-6,14,17-trioxa-2-thia-3,5-diazacyclononadeca[1,2,3-cd]indene-7-carboxylic acid

The title compound was prepared according to the procedure described for Example 82B, substituting Example 91A for Example 82A. ¹H NMR (500 MHz, dimethyl sulfoxide-d₆) δ ppm 2.22 (d, J=6.6 Hz, 6H), 2.51-2.58 (m, 2H), 2.60-2.70 (m, 2H), 2.88 (d, J=16.7 Hz, 1H), 3.21 (s, 3H), 3.35-3.41 (m, 2H), 3.77 (s, 3H), 3.82-3.92 (m, 1H), 4.31 (dd, J=13.1, 8.7 Hz, 1H), 4.47 (d, J=12.9 Hz, 1H), 4.50-4.61 (m, 1H), 5.08-5.25 (m, 2H), 5.63 (d, J=2.9 Hz, 1H), 6.11 (dd, J=5.3, 2.9 Hz, 1H), 6.80 (dd, J=9.0, 3.0 Hz, 1H), 6.91 (d, J=9.1 Hz, 1H), 6.95 (d, J=8.4 Hz, 1H), 7.02-7.08 (m, 1H), 7.11-7.23 (m, 6H), 7.42-7.49 (m, 1H), 7.51-7.57 (m, 2H), 8.74 (s, 1H), 8.88 (d, J=5.1 Hz, 1H). LC/MS (APCI) m/z 892.3 (M+H)⁺.

### Example 92

### (7R,16R,21S)-19-chloro-1-(4-fluorophenyl)-16-({(3R)-3-[(methanesulfonyl)methyl]-4-methylpiperazin-1-yl}methyl)-10-{[2-(2-methoxyphenyl)pyrimidin-4-yl]methoxy}-20-methyl-7,8,15,16-tetrahydro-18,21-etheno-13,9-(metheno)-6,14,17-trioxa-2-thia-3,5-diazacyclononadeca[1,2,3-cd]indene-7-carboxylic acid

### Example 92A

### (R)-1-benzyl 4-tert-butyl 2-(hydroxymethyl)piperazine-1,4-dicarboxylate

To a stirring mixture of (R)-tert-butyl 3-(hydroxymethyl)piperazine-1-carboxylate (3.46 g) and triethylamine (4.46 mL) in dichloromethane (160 mL) was added benzyl chloroformate (2.5 mL) and the reaction mixture was stirred at ambient temperature for 15 minutes. The mixture was concentrated onto silica gel and purification by chromatography on a CombiFlash® Teledyne Isco system using a Teledyne Isco RediSep® Rf gold 120 g silica gel column (eluting with 20-100% ethyl acetate/heptane) provided the title compound. LC/MS (APCI) m/z 351.3 (M+H)⁺.

### Example 92B

### (R)-1-benzyl 4-tert-butyl 2-(((methylsulfonyl)oxy)methyl)piperazine-1,4-dicarboxylate

To a stirred mixture of Example 92A (3.98 g) and triethylamine (4.75 mL) in 4.1 mL of dichloromethane was added methanesulfonyl chloride (1.3 mL) and the mixture was stirred at ambient temperature for 20 minutes. The mixture was concentrated onto silica gel then purification by flash chromatography on a CombiFlash® Teledyne Isco system using a Teledyne Isco RediSep® Rf gold 120 g silica gel column (eluting with 20-100% ethyl acetate/heptane) provided the title compound. LC/MS (APCI) m/z 329.0 (M+H−BOC)⁺.

### Example 92C

### (R)-1-benzyl 4-tert-butyl 2-((methylthio)methyl)piperazine-1,4-dicarboxylate

An 8 mL vial, equipped with a stir bar, was charged with Example 92B (4.7 g) and sodium methanethiolate (2.3 g). The vial was capped with a septa and evacuated and backfilled with nitrogen. N,N-Dimethylformamide (73.1 mL) was added via syringe, and the mixture was evacuated and backfilled with nitrogen again. The mixture was stirred at 45° C. for 60 minutes, cooled to ambient temperature, and poured into a separatory funnel containing 500 mL of water. The aqueous mixture was extracted with two portions of diethyl ether and the combined organic layers were dried over anhydrous magnesium sulfate, filtered and concentrated onto silica gel. Purification by flash chromatography on a CombiFlash® Teledyne Isco system using a Teledyne Isco RediSep® Rf gold 220 g silica gel column (eluting 5-60% ethyl acetate/heptanes) provided the title compound. LC/MS (APCI) m/z 381.3 (M+H)⁺.

### Example 92D

### (R)-1-benzyl 4-tert-butyl 2-((methylsulfonyl)methyl)piperazine-1,4-dicarboxylate

Example 92C (2.8 g) was dissolved in methanol (147 mL) and the mixture was stirred in an ice bath. Potassium peroxomonosulfate (6.79 g) was added in one portion, the cooling bath was removed and the mixture allowed to stir at ambient temperature for 2 hours. The methanol was then evaporated and the resulting mixture was diluted with ethyl acetate and poured into a separatory funnel. The organic mixture was washed with water and brine, dried over anhydrous magnesium sulfate, filtered and concentrated onto silica gel. Purification by flash chromatography on a CombiFlash® Teledyne Isco system using a Teledyne Isco RediSep® Rf gold 80 g silica gel column (eluting with 20-100% ethyl acetate/heptane) provided the title compound. LC/MS (APCI) m/z 413.2 (M+H)⁺.

### Example 92E

### (R)-tert-butyl 3-((methylsulfonyl)methyl)piperazine-1-carboxylate

Example 92D (2.25 g) was dissolved in methanol (54.5 mL) and palladium hydroxide on carbon (0.766 g, 20% wt on carbon Degussa® type) was added. The reaction mixture was evacuated and backfilled with nitrogen twice then evacuated and backfilled with hydrogen. The reaction mixture was stirred under hydrogen (used hydrogen balloon) at room temperature for 3 hours. The mixture was filtered through a diatomaceous earth pad, concentrated, filtered again through a PTFE membrane and concentrated to provide the title compound. The crude amine was carried through the next step without additional purification. LC/MS (APCI) m/z 279.3 (M+H)⁺.

### Example 92F

### (R)-tert-butyl 4-methyl-3-((methylsulfonyl)methyl)piperazine-1-carboxylate

Example 92E (95 mg) was dissolved in tetrahydrofuran (3.4 mL) and 37% aqueous formaldehyde (76 μL) and sodium triacetoxyborohydride (217 mg) were added. The mixture was stirred at ambient temperature for 2 hours. The mixture was concentrated onto silica gel and purification by flash chromatography on a CombiFlash® Teledyne Isco system using a Teledyne Isco RediSep® Rf gold 12 g silica gel column (eluting with 50-100% 2:1 ethanol:ethyl acetate/heptane) provided the title compound. LC/MS (APCI) m/z 293.2 (M+H)⁺.

### Example 92G

### (R)-1-methyl-2-((methylsulfonyl)methyl)piperazine

Example 92F (95 mg) was dissolved in dichloromethane (1.0 mL) and 1 mL of trifluoroacetic acid was added. The mixture was stirred at ambient temperature for 15 minutes and was concentrated to give the crude trifluoroacetic acid salt. A 20G MEGA BE-SCX Bond Elut® resin cartridge was first washed with 50% methanol/dichloromethane (50 mL) and the crude residue obtained was loaded as a 1:1 methanol:dichloromethane mixture (˜2 mL). The resin was washed with 50% methanol/dichloromethane (50 mL). The filtrate was removed and was replaced with an empty collecting flask. The cartridge was washed with 200 mL of a 2 molar ammonium hydroxide in methanol mixture. The filtrate was concentrated to provide the title compound as a free base. LC/MS (APCI) m/z 193.4 (M+H)⁺.

### Example 92H

### ethyl (7R,16R,21S)-19-chloro-1-(4-fluorophenyl)-16-({(3R)-3-[(methanesulfonyl)methyl]-4-methylpiperazin-1-yl}methyl)-10-{[2-(2-methoxyphenyl)pyrimidin-4-yl]methoxy}-20-methyl-7,8,15,16-tetrahydro-18,21-etheno-9,13-(metheno)-6,14,17-trioxa-2-thia-3,5-diazacyclononadeca[1,2,3-cd]indene-7-carboxylate

Example 92H was synthesized according to the procedure described for Example 73J, substituting Example 92G for 1-methylpiperazine. LC/MS (APCI) m/z 1023.2 (M+H)⁺.

### Example 92I

### (7R,16R,21S)-19-chloro-1-(4-fluorophenyl)-16-({(3R)-3-[(methanesulfonyl)methyl]-4-methylpiperazin-1-yl}methyl)-10-{[2-(2-methoxyphenyl)pyrimidin-4-yl]methoxy}-20-methyl-7,8,15,16-tetrahydro-18,21-etheno-13,9-(metheno)-6,14,17-trioxa-2-thia-3,5-diazacyclononadeca[1,2,3-cd]indene-7-carboxylic acid

Example 92I was synthesized according to the procedure described for Example 82B, substituting Example 92H for Example 82A. ¹H NMR (500 MHz, dimethyl sulfoxide-d₆) δ ppm 2.22 (s, 3H), 2.57-3.05 (m, 10H), 3.15 (s, 3H), 3.20-3.30 (m, 2H), 3.38-3.64 (m, 2H), 3.77 (s, 3H), 3.80-3.87 (m, 2H), 4.38 (dd, J=13.3, 8.7 Hz, 1H), 4.50 (d, J=13.0 Hz, 1H), 4.63-4.75 (m, 1H), 5.12-5.25 (m, 2H), 5.68 (d, J=2.8 Hz, 1H), 6.19 (dd, J=5.0, 3.2 Hz, 1H), 6.85 (dd, J=9.0, 2.9 Hz, 1H), 6.91 (d, J=9.1 Hz, 1H), 6.96 (d, J=8.3 Hz, 1H), 7.06 (t, J=7.4 Hz, 1H), 7.11-7.23 (m, 6H), 7.44-7.50 (m, 1H), 7.52-7.58 (m, 2H), 8.76 (s, 1H), 8.89 (dd, J=5.2, 1.5 Hz, 1H). LC/MS (APCI) m/z 995.2 (M+H)⁺.

### Example 93

### (7R,16R,21S)-19-chloro-1-(4-fluorophenyl)-16-({(3R)-3-[(methanesulfonyl)methyl]piperazin-1-yl}methyl)-10-{[2-(2-methoxyphenyl)pyrimidin-4-yl]methoxy}-20-methyl-7,8,15,16-tetrahydro-18,21-etheno-13,9-(metheno)-6,14,17-trioxa-2-thia-3,5-diazacyclononadeca[1,2,3-cd]indene-7-carboxylic acid

### Example 93A

### (R)-2-((methylsulfonyl)methyl)piperazine

Example 93A was synthesized according to the procedure described for Example 92G, substituting Example 92E for Example 92F. LC/MS (APCI) m/z 179.2 (M+H)⁺.

### Example 93B

### (7R,16R,21S)-19-chloro-1-(4-fluorophenyl)-16-({(3R)-3-[(methanesulfonyl)methyl]piperazin-1-yl}methyl)-10-{[2-(2-methoxyphenyl)pyrimidin-4-yl]methoxy}-20-methyl-7,8,15,16-tetrahydro-18,21-etheno-9,13-(metheno)-6,14,17-trioxa-2-thia-3,5-diazacyclononadeca[1,2,3-cd]indene-7-carboxylate

Example 93B was synthesized according to the procedure described for Example 73J, substituting Example 92A for 1-methylpiperazine. LC/MS (APCI) m/z 1010.1 (M+H)⁺.

### Example 93C

### (7R,16R,21S)-19-chloro-1-(4-fluorophenyl)-16-({(3R)-3-[(methanesulfonyl)methyl]piperazin-1-yl}methyl)-10-{[2-(2-methoxyphenyl)pyrimidin-4-yl]methoxy}-20-methyl-7,8,15,16-tetrahydro-18,21-etheno-13,9-(metheno)-6,14,17-trioxa-2-thia-3,5-diazacyclononadeca[1,2,3-cd]indene-7-carboxylic acid

Example 93C was synthesized according to the procedure described for Example 82B, substituting Example 93B for Example 82A. ¹H NMR (500 MHz, dimethyl sulfoxide-d₆) δ ppm 2.21 (s, 3H), 2.52-2.64 (m, 1H), 2.69-3.10 (m, 7H), 3.16 (s, 3H), 3.18-3.24 (m, 1H), 3.25-3.36 (m, 2H), 3.46 (dd, J=14.6, 4.7 Hz, 1H), 3.55-3.68 (m, 2H), 3.77 (s, 3H), 3.79-3.85 (m, 2H), 4.37 (dd, J=13.3, 8.7 Hz, 1H), 4.50 (d, J=12.9 Hz, 1H), 4.61-4.70 (m, 1H), 5.13 (d, J=15.1 Hz, 1H), 5.21 (d, J=15.0 Hz, 1H), 5.67 (d, J=2.7 Hz, 1H), 6.17-6.21 (m, 1H), 6.87 (d, J=3.0 Hz, 1H), 6.90 (d, J=9.1 Hz, 1H), 6.95 (d, J=8.3 Hz, 1H), 7.06 (t, J=7.5 Hz, 1H), 7.11-7.25 (m, 6H), 7.47 (ddd, J=8.7, 7.4, 1.8 Hz, 1H), 7.52-7.58 (m, 2H), 8.75 (s, 1H), 8.89 (dd, J=5.1, 1.5 Hz, 1H). LC/MS (APCI) m/z 981.2 (M+H)⁺.

### Example 94

### (7R,16R,21S)-19-chloro-16-[(1,1-dioxo-1λ⁶-thiomorpholin-4-yl)methyl]-1-(4-fluorophenyl)-10-{[2-(2-methoxyphenyl)pyrimidin-4-yl]methoxy}-20-methyl-7,8,15,16-tetrahydro-18,21-etheno-13,9-(metheno)-6,14,17-trioxa-2-thia-3,5-diazacyclononadeca[1,2,3-cd]indene-7-carboxylic acid

### Example 94A

### ethyl (7R,16R,21S)-19-chloro-16-[(1,1-dioxo-1λ⁶-thiomorpholin-4-yl)methyl]-1-(4-fluorophenyl)-10-{[2-(2-methoxyphenyl)pyrimidin-4-yl]methoxy}-20-methyl-7,8,15,16-tetrahydro-18,21-etheno-9,13-(metheno)-6,14,17-trioxa-2-thia-3,5-diazacyclononadeca[1,2,3-cd]indene-7-carboxylate

Example 94A was synthesized according to the procedure described for Example 73J, substituting thiomorpholine 1,1-dioxide for 1-methylpiperazine. LC/MS (APCI) m/z 965.9 (M+H)⁺.

### Example 94B

### (7R,16R,21S)-19-chloro-16-[(1,1-dioxo-1λ⁶-thiomorpholin-4-yl)methyl]-1-(4-fluorophenyl)-10-{[2-(2-methoxyphenyl)pyrimidin-4-yl]methoxy}-20-methyl-7,8,15,16-tetrahydro-18,21-etheno-13,9-(metheno)-6,14,17-trioxa-2-thia-3,5-diazacyclononadeca[1,2,3-cd]indene-7-carboxylic acid

Example 94B was synthesized according to the procedure described for Example 82B, substituting Example 94A for Example 82A. ¹H NMR (500 MHz, dimethyl sulfoxide-d₆) δ ppm 2.23 (s, 3H), 2.75-3.28 (m, 11H), 3.77 (s, 3H), 3.88 (dd, J=17.1, 5.4 Hz, 1H), 4.35 (dd, J=13.2, 8.6 Hz, 1H), 4.51 (d, J=12.9 Hz, 1H), 4.54-4.64 (m, 1H), 5.10-5.28 (m, 2H), 5.66 (d, J=2.5 Hz, 1H), 6.16 (dd, J=5.2, 2.9 Hz, 1H), 6.87-6.93 (m, 2H), 6.96 (d, J=8.3 Hz, 1H), 7.06 (t, J=7.4 Hz, 1H), 7.13-7.22 (m, 6H), 7.44-7.50 (m, 1H), 7.52 (d, J=5.2 Hz, 1H), 7.55 (dd, J=7.5, 1.8 Hz, 1H), 8.75 (s, 1H), 8.88 (d, J=5.1 Hz, 1H). LC/MS (APCI) m/z 938.0 (M+H)⁺.

### Example 95

### (7R,16R,21S)-19-chloro-1-(4-fluorophenyl)-10-{[2-(2-methoxyphenyl)pyrimidin-4-yl]methoxy}-20-methyl-16-[(4-methyl-3-oxopiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid

The title compound was prepared using the conditions described in Example 73J and Example 82B substituting 1-methylpiperazin-2-one for 1-[2-(methylsulfonyl)ethyl]piperazine. ¹H NMR (400 MHz, dimethyl sulfoxide-d₆) δ ppm 8.88 (d, J=5.1 Hz, 1H), 8.73 (s, 1H), 7.59-7.50 (m, 2H), 7.47 (ddd, J=9.0, 7.3, 1.8 Hz, 1H), 7.26-7.11 (m, 6H), 7.06 (td, J=7.5, 1.0 Hz, 1H), 6.96 (d, J=8.3 Hz, 1H), 6.90 (d, J=9.0 Hz, 1H), 6.81 (dd, J=9.0, 2.9 Hz, 1H), 6.12 (dd, J=5.3, 2.9 Hz, 1H), 5.65 (d, J=2.8 Hz, 1H), 5.17 (q, J=15.0 Hz, 2H), 4.59 (q, J=6.5 Hz, 1H), 4.46 (d, J=12.9 Hz, 1H), 4.35 (dd, J=13.2, 8.6 Hz, 1H), 3.87 (dd, J=16.9, 5.4 Hz, 1H), 3.77 (s, 3H), 3.25-2.84 (m, 5H), 2.81 (s, 3H), 2.71-2.61 (m, 4H), 2.23 (s, 3H). MS (ESI) m/z 917.0 (M+H)⁺.

### Example 96

### (7R,20S)-18-chloro-1-(4-fluorophenyl)-19-methyl-15-[2-(4-methylpiperazin-1-yl)ethyl]-10-({2-[(1R,5S)-3-oxa-8-azabicyclo[3.2.1]octan-8-yl]pyrimidin-4-yl}methoxy)-7,8,15,16-tetrahydro-14H-17,20-etheno-13,9-(metheno)-6-oxa-2-thia-3,5,15-triazacyclooctadeca[1,2,3-cd]indene-7-carboxylic acid

### Example 96A

### methyl 2-((1R,5S)-3-oxa-8-azabicyclo[3.2.1]octan-8-yl)pyrimidine-4-carboxylate

The title compound was prepared by substituting (1R,5S)-3-oxa-8-azabicyclo[3.2.1]octane hydrochloride for (1S,4S)-2-oxa-5-azabicyclo[2.2.1]heptane in Example 89A. MS (DCI) m/z 250.0 (M+H)⁺.

### Example 96B

### (2-((1R,5S)-3-oxa-8-azabicyclo[3.2.1]octan-8-yl)pyrimidin-4-yl)methanol

The title compound was prepared by substituting Example 96A for Example 89A in Example 89B. MS (DCI) m/z 222.0 (M+H)⁺.

### Example 96C

### (2-((1R,5S)-3-oxa-8-azabicyclo[3.2.1]octan-8-yl)pyrimidin-4-yl)methyl methanesulfonate

The title compound was prepared by substituting Example 96B for Example 89B in Example 89C. MS (DCI) m/z 299.9 (M+H)⁺.

### Example 96D

### ethyl (7R,20S)-18-chloro-1-(4-fluorophenyl)-19-methyl-15-[2-(4-methylpiperazin-1-yl)ethyl]-10-({2-[(1R,5S)-3-oxa-8-azabicyclo[3.2.1]octan-8-yl]pyrimidin-4-yl}methoxy)-7,8,15,16-tetrahydro-14H-17,20-etheno-13,9-(metheno)-6-oxa-2-thia-3,5,15-triazacyclooctadeca[1,2,3-cd]indene-7-carboxylate

The title compound was prepared by substituting Example 96C for Example 65E in Example 65N. MS (ESI) m/z 933.2 (M+H)⁺.

### Example 96E

### (7R,20S)-18-chloro-1-(4-fluorophenyl)-19-methyl-15-[2-(4-methylpiperazin-1-yl)ethyl]-10-({2-[(1R,5S)-3-oxa-8-azabicyclo[3.2.1]octan-8-yl]pyrimidin-4-yl}methoxy)-7,8,15,16-tetrahydro-14H-17,20-etheno-13,9-(metheno)-6-oxa-2-thia-3,5,15-triazacyclooctadeca[1,2,3-cd]indene-7-carboxylic acid

The title compound was prepared by substituting Example 96D for Example 65N in Example 65O. ¹H NMR (400 MHz, dimethylsulfoxide-d₆) δ ppm 8.66 (s, 1H), 8.07 (d, 1H), 7.53 (d, 1H), 7.32 (d, 1H), 7.22 (m, 3H), 7.14 (m, 2H), 6.79 (d, 1H), 6.54 (s, 1H), 6.38 (s, 1H), 5.94 (m, 1H), 4.97 (d, 1H), 4.83 (d, 1H), 4.55 (br s, 3H), 4.42 (m, 2H), 4.22 (br s, 3H), 3.56 (m, 8H), 3.21 (m, 2H), 3.08 (m, 6H), 2.81 (s, 3H), 1.94 (m, 2H), 1.85 (m, 2H), 1.67 (s, 3H). MS (ESI) m/z 903.1 (M−H)⁻.

### Example 97

### (7R,20S)-18-chloro-10-{[2-(2,6-dioxa-9-azaspiro[4.5]decan-9-yl)pyrimidin-4-yl]methoxy}-1-(4-fluorophenyl)-19-methyl-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylic acid

### Example 97A

### methyl 2-(2,6-dioxa-9-azaspiro[4.5]decan-9-yl)pyrimidine-4-carboxylate

The title compound was prepared by substituting 2,6-dioxa-9-azaspiro[4.5]decane for (1S,4S)-2-oxa-5-azabicyclo[2.2.1]heptane in Example 89A. MS (DCI) m/z 280.0 (M+H)⁺.

### Example 97B

### (2-(2,6-dioxa-9-azaspiro[4.5]decan-9-yl)pyrimidin-4-yl)methanol

The title compound was prepared by substituting Example 97A for Example 89A in Example 89B. MS (DCI) m/z 252.0 (M+H)⁺.

### Example 97C

### (2-(2,6-dioxa-9-azaspiro[4.5]decan-9-yl)pyrimidin-4-yl)methyl methanesulfonate

The title compound was prepared by substituting Example 97B for Example 89B in Example 89C. MS (ESI) m/z 329.7 (M+H)⁺.

### Example 97D

### ethyl (7R,20S)-18-chloro-10-{[2-(2,6-dioxa-9-azaspiro[4.5]decan-9-yl)pyrimidin-4-yl]methoxy}-1-(4-fluorophenyl)-19-methyl-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylate

The title compound was prepared by substituting Example 97C for Example 65E in Example 65N. MS (ESI) m/z 963.5 (M+H)⁺.

### Example 97E

### (7R,20S)-18-chloro-10-{[2-(2,6-dioxa-9-azaspiro[4.5]decan-9-yl)pyrimidin-4-yl]methoxy}-1-(4-fluorophenyl)-19-methyl-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylic acid

The title compound was prepared by substituting Example 97D for Example 65N in Example 65O. ¹H NMR (400 MHz, dimethylsulfoxide-d₆) δ ppm 8.64 (s, 1H), 8.09 (dd, 1H), 7.50 (d, 1H), 7.30 (d, 1H), 7.22 (m, 3H), 7.14 (m, 2H), 6.76 (d, 1H), 6.53 (s, 1H), 6.40 (dd, 1H), 5.90 (dd, 1H), 4.97 (d, 1H), 4.79 (d, 1H), 4.32 (v br s, 2H), 4.18 (v br s, 2H), 3.78 (m, 4H), 3.71 (s, 2H), 3.66 (m, 8H), 3.57 (m, 4H), 3.21 (m, 2H), 3.08 (m, 4H), 2.79 (s, 3H), 1.97 (m, 1H), 1.83 (m, 1H), 1.68 (s, 3H). MS (ESI) m/z 935.2 (M+H)⁺.

### Example 98

### (7R,20S)-10-{[2-(bicyclo[1.1.1]pentan-1-yl)pyrimidin-4-yl]methoxy}-18-chloro-1-(4-fluorophenyl)-19-methyl-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylic acid

### Example 98A

### bicyclo[1.1.1]pentane-1-carboxamide

To a mixture of bicyclo[1.1.1]pentane-1-carboxylic acid (4 g) in dichloromethane (40 mL) was added thionyl chloride (4.7 mL). The reaction mixture was heated to reflux for 18 hours. The mixture was cooled to 0° C. and was added to aqueous ammonium hydroxide (9 mL) at 0° C. for 30 minutes. The resulting mixture was filtered to provide the title compound which was used in the next step without further purification. ¹H NMR (400 MHz, dimethyl sulfoxide-d₆) δ ppm 7.16 (br s, 1H), 6.85 (br s, 1H), 2.37-2.32 (m, 1H), 1.89 (s, 6H).

### Example 98B

### methyl bicyclo[1.1.1]pentane-1-carbimidate

To a mixture of Example 98A (4 g) in dichloromethane (2 L) was added trimethyloxonium tetrafluoroborate (13.3 g) at 0° C. and the reaction mixture was stirred at 25° C. for 16 hours under a nitrogen atmosphere. The resulting mixture was treated with saturated aqueous sodium bicarbonate to pH 8 and was separated. The aqueous layer was extracted with dichloromethane (2×50 mL). The combined organic layers were washed with brine (50 mL), dried over Na₂SO₄, filtered and concentrated under vacuum to provide the title compound which was used in the next step without further purification. ¹H NMR (400 MHz, CDCl₃) δ ppm 6.88 (br s, 1H), 3.68 (s, 3H), 2.42 (s, 1H), 2.01-1.93 (m, 6H).

### Example 98C

### bicyclo[1.1.1]pentane-1-carboximidamide hydrochloride

To a mixture of Example 98B (6 g) in methanol (60 mL) was added ammonium chloride (2.9 g). The reaction mixture was stirred at 70° C. for 18 hours. The resulting mixture was filtered and cooled to at 0° C., and was treated with 4M HCl in methanol until pH=2. The mixture was concentrated under reduced pressure. The residue was triturated with dichloromethane (20 mL) to provide the title compound. ¹H NMR (400 MHz, dimethyl sulfoxide-d₆) δ ppm 8.98 (br d, J=11.5 Hz, 4H), 2.48-2.46 (m, 1H), 2.11 (s, 6H).

### Example 98D

### 2-(bicyclo[1.1.1]pentan-1-yl)-4-(dimethoxymethyl)pyrimidine

To a mixture of Example 98C (6 g) in methanol (60 mL) was added sodium methanolate (61.4 mL, 123 mmol). After 10 minutes, (E)-4-(dimethylamino)-1,1-dimethoxybut-3-en-2-one (10.6 g, 61.4 mmol) was added and the reaction mixture was heated to 70° C. for 18 hours under nitrogen. The reaction mixture was concentrated under vacuum. The resulting residue was diluted with water (100 mL) and extracted with dichloromethane (2×150 mL). The combined organic layers were washed with brine (200 mL), dried over Na₂SO₄, filtered and concentrated under vacuum. The residue was purified by column chromatography on silica gel (petroleum:ethylacetate=30:1 to 5:1) to provide the title compound. ¹H NMR (400 MHz, CDCl₃) δ ppm 8.66 (d, J=5.1 Hz, 1H), 7.31 (d, J=5.1 Hz, 1H), 5.19 (s, 1H), 3.44-3.31 (s, 6H), 2.49 (s, 1H), 2.20 (s, 6H).

### Example 98E

### (2-(bicyclo[1.1.1]pentan-1-yl)pyrimidin-4-yl)methanol

To a mixture of Example 98D (8.5 g) in 1,4-dioxane (190 mL) was added an aqueous hydrogen chloride mixture (193 mL, 4 N) in portions, at 15° C. The mixture was stirred at 60° C. for 18 hours. The reaction mixture was cooled to 0° C. and sodium hydroxide (26.2 g) was added portionwise at 0° C. The pH of the reaction mixture was then adjusted to 8 using 30% aqueous sodium hydroxide mixture. To the resulting mixture was added sodium borohydride (2.9 g) in portions with stirring for 2 hours at 0° C. The reaction mixture was extracted with ethyl acetate (3×100 mL). The combined organic layers were washed with brine (100 mL), dried over Na₂SO₄, filtered and concentrated under vacuum. The residue was purified by column chromatography on silica gel (eluted with petroleum ether/ethyl acetate from 100:1 to 3:1) to provide the title compound. ¹H NMR (400 MHz, CDCl₃) δ ppm 8.60 (d, J=5.1 Hz, 1H), 7.11 (d, J=5.3 Hz, 1H), 4.71 (d, J=4.0 Hz, 2H), 3.88 (t, J=4.4 Hz, 1H), 2.57-2.53 (m, 1H), 2.29-2.19 (m, 6H). LC/MS (ESI) m/z 177.1 (M+H)⁺.

### Example 98F

### (7R,20S)-10-{[2-(bicyclo[1.1.1]pentan-1-yl)pyrimidin-4-yl]methoxy}-18-chloro-1-(4-fluorophenyl)-19-methyl-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylic acid

The title compound was prepared according to the protocols for Example 84H-J, substituting Example 98E for Example 84G. ¹H NMR (400 MHz, dimethyl sulfoxide-d₆) δ ppm 8.64 (s, 1H), 8.51 (d, J=5.1 Hz, 1H), 7.52 (d, J=7.9 Hz, 1H), 7.30 (d, J=7.9 Hz, 1H), 7.27-7.19 (m, 3H), 7.18-7.09 (m, 3H), 6.84 (d, J=8.6 Hz, 1H), 6.48 (d, J=2.2 Hz, 1H), 5.92 (dd, J=8.5, 4.7 Hz, 1H), 5.17-4.94 (m, 4H), 4.36 (t, J=14.7 Hz, 3H), 4.19 (s, 3H), 3.26-2.99 (m, 8H), 2.81 (s, 3H), 2.17-2.12 (m, 6H), 1.75 (s, 3H), 1.25 (d, J=12.3 Hz, 2H). MS (ESI) m/z 861 (M+H)⁺.

### Example 99

### (7R,20S)-18-chloro-1-(4-fluorophenyl)-19-methyl-10-({2-[(4-methyloxan-4-yl)methyl]pyrimidin-4-yl}methoxy)-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylic acid

### Example 99A

### ethyl 2-(tetrahydro-4H-pyran-4-ylidene)acetate

To a mixture of sodium hydride (24 g) in toluene (250 mL) was added ethyl 2-(diethoxyphosphoryl) acetate (134 g) at 0° C. After stirring under nitrogen for 30 minutes at 0° C., tetrahydro-4H-pyran-4-one (30 g) was added and the mixture was stirred at 25° C. for 12 hours. The reaction mixture was quenched by addition of aqueous NH₄Cl (1 L) at 0° C. The aqueous layer was extracted with ethyl acetate (2×1 L). The combined organic layers were dried over Na₂SO₄, filtered and concentrated to give a residue which was purified by column chromatography on silica gel (petro ether:ethyl acetate=10:1) to provide the title compound. ¹H NMR (400 MHz, dimethyl sulfoxide-d₆) δ ppm 5.72 (s, 1H), 4.07 (q, J=7.2 Hz, 2H), 3.64 (td, J=5.4, 17.6 Hz, 4H), 2.88 (br t, J=5.2 Hz, 2H), 2.29 (br t, J=5.1 Hz, 2H), 1.19 (t, J=7.1 Hz, 3H).

### Example 99B

### ethyl 2-(4-methyltetrahydro-2H-pyran-4-yl)acetate

To a suspension of copper(I) iodide (63.8 g) in ether (200 mL) at 0° C. was added a mixture of methyllithium in ethyl ether (419 mL, 1.6 M) in portions. The reaction mixture was stirred at 0° C. for 10 minutes. The solvent was evaporated under reduced pressure. Dichloromethane (200 mL) was added under nitrogen at 0° C. The mixture was stirred at 0° C. for 10 minutes. The solvent was evaporated again. Dichloromethane (200 mL) was added under nitrogen at 0° C. The mixture was stirred at 0° C. for 10 minutes. To the mixture was added chlorotrimethylsilane (36.4 g) and a mixture of Example 99A (30 g) in dichloromethane (200 mL) at −78° C. The reaction mixture was stirred at 0° C. for 12 hours. The mixture was quenched by addition of aqueous saturated NH₄Cl mixture (250 mL) and was extracted with dichloromethane (3×250 mL). The combined organic layers were washed with brine (500 mL), dried over Na₂SO₄, filtered and concentrated under vacuum to provide a residue which was purified by column chromatography on silica gel (petroleum:ethyl acetate=30:1˜5:1) to provide the title compound. ¹H NMR (400 MHz, CDCl₃) δ ppm 4.12 (q, J=7.1 Hz, 2H), 3.76-3.59 (m, 4H), 2.30 (s, 2H), 1.66-1.56 (m, 2H), 1.49-1.40 (m, 2H), 1.25 (t, J=7.2 Hz, 3H), 1.12 (s, 3H).

### Example 99C

### 2-(4-methyltetrahydro-2H-pyran-4-yl)acetic acid

To a mixture of Example 99B (20 g) in ethanol (80 mL), tetrahydrofuran (80 mL) and water (20 mL) was added sodium hydroxide (11.6 g) at 0° C. The reaction mixture was stirred at 25° C. for 12 hours. The mixture was concentrated and diluted with water (200 mL). The aqueous layer was extracted with ethyl acetate (2×150 mL). The pH of the aqueous layer was adjusted to 1 with 4 M aqueous HCl. The aqueous layer was extracted with ethyl acetate (2×250 mL). The combined organic layers were dried over Na₂SO₄, filtered and concentrated to provide the title compound. ¹H NMR (400 MHz, CDCl₃) δ ppm 11.08 (br s, 1H), 3.79-3.61 (m, 4H), 2.36 (s, 2H), 1.72-1.60 (m, 2H), 1.54-1.44 (m, 2H), 1.17 (s, 3H).

### Example 99D

### 2-(4-methyltetrahydro-2H-pyran-4-yl)acetyl chloride

A mixture of Example 99C (15 g) in thionyl chloride (60 mL) was stirred at 80° C. for 12 hours. The mixture was cooled to 25° C. The mixture was concentrated to provide the title compound. ¹H NMR (400 MHz, CDCl₃) δ ppm 3.76-3.60 (m, 4H), 2.95 (s, 2H), 1.64 (ddd, J=4.3, 8.7, 13.4 Hz, 2H), 1.51 (td, J=4.2, 13.3 Hz, 2H), 1.22 (s, 3H).

### Example 99E

### 2-(4-methyltetrahydro-2H-pyran-4-yl)acetamide

To a mixture of Example 99D (16.5 g) in dichloromethane (120 mL) was added ammonium hydroxide (90 mL) at 0° C. The reaction mixture was stirred at 25° C. for 3 hours. The mixture was separated and the water layer was extracted with dichloromethane (2×150 mL). The combined organic layers were washed with brine (100 mL), dried over Na₂SO₄, filtered and concentrated under vacuum to provide the title compound. ¹H NMR (400 MHz, CDCl₃) δ ppm 5.62-5.14 (m, 2H), 3.85-3.56 (m, 4H), 2.20 (s, 2H), 1.67 (ddd, J=4.3, 8.7, 17.8 Hz, 2H), 1.49 (td, J=3.7, 13.7 Hz, 2H), 1.18 (s, 3H).

### Example 99F

### methyl 2-(4-methyltetrahydro-2H-pyran-4-yl)acetimidate

To a mixture of Example 99E (12 g) in dichloromethane (150 mL) was added trimethyloxonium tetrafluoroborate (16 g) at 0° C. The reaction mixture was stirred at 20° C. for 12 hours. The mixture was quenched by addition of saturated aqueous NaHCO₃ (150 mL). The mixture was separated and the water layer was extracted with dichloromethane (3×150 mL). The combined organic layers were washed with brine (150 mL), dried over Na₂SO₄, filtered and concentrated under vacuum to provide the title compound. ¹H NMR (400 MHz, CDCl₃) δ ppm 6.87 (br s, 1H), 3.76-3.61 (m, 7H), 2.25 (s, 2H), 1.57 (ddd, J=4.2, 8.9, 13.4 Hz, 2H), 1.39 (td, J=3.7, 13.6 Hz, 2H), 1.11-1.03 (m, 3H).

### Example 99G

### 2-(4-methyltetrahydro-2H-pyran-4-yl)acetimidamide hydrochloride

To a mixture of Example 99F (9 g) in methanol (100 mL) was added ammonium chloride (4 g) at 0° C. The mixture was stirred at 25° C. for 12 hours. The mixture was concentrated to give a residue. The residue was diluted with dichloromethane (50 mL). The mixture was filtered and the filter cake was washed with methanol (100 mL) to provide the title compound. ¹H NMR (400 MHz, dimethyl sulfoxide-d₆) δ ppm 8.91 (br s, 4H), 3.64 (td, J=4.1, 11.8 Hz, 2H), 3.54-3.43 (m, 2H), 2.35 (s, 2H), 1.61-1.48 (m, 2H), 1.26 (br d, J=13.5 Hz, 2H), 1.06 (s, 3H).

### Example 99H

### 4-(dimethoxymethyl)-2-((4-methyltetrahydro-2H-pyran-4-yl)methyl)pyrimidine

To a mixture of Example 99G (6 g) in methanol (30 mL) were added (E)-4-(dimethylamino)-1,1-dimethoxybut-3-en-2-one (6.15 g) and sodium methanolate (29.6 mL) at 25° C. The reaction mixture was stirred in 80° C. oil bath for 12 hours. The mixture was concentrated and diluted with water (50 mL). The mixture was extracted with ethyl acetate (2×50 mL). The combined organic layers were dried over Na₂SO₄, filtered and concentrated to give a residue which was purified by column chromatography on silica gel (petroleum:ethyl acetate=15:1˜5:1) to provide the title compound. ¹H NMR (400 MHz, CDCl₃) δ ppm 8.71 (d, J=5.1 Hz, 1H), 7.38 (d, J=5.1 Hz, 1H), 5.26 (s, 1H), 3.84-3.76 (m, 2H), 3.66 (ddd, J=3.3, 8.5, 11.7 Hz, 2H), 3.41 (s, 6H), 3.02 (s, 2H), 1.69 (ddd, J=4.0, 8.8, 13.2 Hz, 2H), 1.40 (td, J=4.0, 14.1 Hz, 2H), 1.04 (s, 3H).

### Example 99I

### (2-((4-methyltetrahydro-2H-pyran-4-yl)methyl)pyrimidin-4-yl)methanol

To a mixture of Example 99H (4 g) in dioxane (25 mL) was added hydrogen chloride (25 mL) at 25° C. The reaction mixture was stirred at 60° C. for 12 hours. The reaction mixture was cooled to room temperature and the pH of the reaction mixture was adjusted to 8 by addition of 2M aqueous NaOH. Sodium borohydride (1.08 g) was added to the reaction mixture in portions at 0° C. The reaction mixture was stirred at 0° C. for 2 hours. The mixture was concentrated to give a residue. The residue was diluted with water (25 mL) and extracted with ethyl acetate (3×25 mL). The combined organic layers were washed with brine (50 mL), dried over Na₂SO₄, filtered and concentrated under vacuum. The resulting residue was purified by column chromatography on silica gel (petroleum:ethyl acetate=30:1˜3:1) to provide the title compound. ¹H NMR (400 MHz, dimethyl sulfoxide-d₆) δ ppm 8.69 (d, J=5.1 Hz, 1H), 7.38 (d, J=5.1 Hz, 1H), 5.57 (t, J=5.9 Hz, 1H), 4.51 (d, J=5.9 Hz, 2H), 3.72-3.61 (m, 2H), 3.59-3.47 (m, 2H), 2.84 (s, 2H), 1.59-1.47 (m, 2H), 1.28 (ddd, J=3.4, 5.9, 13.4 Hz, 2H), 0.94 (s, 3H). LC/MS (ESI) m/z 223 (M+H)⁺.

### Example 99J

### (7R,20S)-18-chloro-1-(4-fluorophenyl)-19-methyl-10-({2-[(4-methyloxan-4-yl)methyl]pyrimidin-4-yl}methoxy)-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylic acid

The title compound was prepared as described for Example 84H-J, substituting Example 99I for Example 84G. ¹H NMR (400 MHz, dimethyl sulfoxide-d₆) δ ppm 8.64 (s, 1H), 8.53 (d, J=5.2 Hz, 1H), 7.51 (d, J=7.9 Hz, 1H), 7.30 (d, J=7.9 Hz, 1H), 7.22 (qd, J=7.2, 6.4, 2.6 Hz, 3H), 7.18-7.10 (m, 3H), 6.80 (d, J=8.5 Hz, 1H), 6.52 (d, J=2.0 Hz, 1H), 5.91 (dd, J=9.5, 4.2 Hz, 1H), 5.13 (d, J=14.8 Hz, 2H), 4.95 (d, J=14.7 Hz, 2H), 4.34 (d, J=17.1 Hz, 3H), 4.18 (s, 3H), 3.31-2.96 (m, 12H), 2.80 (s, 3H), 1.69 (s, 3H), 1.50 (ddt, J=12.1, 7.7, 3.7 Hz, 4H), 1.26 (ddt, J=14.3, 6.3, 3.9 Hz, 4H), 0.91 (s, 3H). MS (ESI) m/z 907 (M+H)⁺.

### Example 100

### (7R,20S)-18-chloro-10-{[2-(2-cyanophenyl)pyrimidin-4-yl]methoxy}-1-(4-fluorophenyl)-19-methyl-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylic acid

### Example 100A

### (E)-4-(dimethylamino)-1,1-dimethoxybut-3-en-2-one

1,1-Dimethoxy-N,N-dimethylmethanamine (15 g) and 1,1-dimethoxypropan-2-one (14.9 g) were mixed in a 250 mL flask and the mixture was stirred at 110° C. for 3 hours. Thin layer chromatography showed the starting material was consumed. The formed methanol was removed continuously via distillation. The reaction mixture was distilled under high vacuum (decreasing the pressure slowly to 30 mbar) to remove by-products and starting materials. The remaining crude product was distilled at 0.1 mbar. Fractions were collected between 107˜118° C. head temperature (bath temperature 160-165° C.) to provide the title compound. ¹H NMR (400 MHz, dimethyl sulfoxide-d₆) δ ppm 2.78 (s, 3H), 3.09 (s, 3H), 3.26 (s, 6H), 4.42 (s, 1H), 5.18 (d, J=12.35 Hz, 1H), 7.59 (d, J=12.79 Hz, 1H).

### Example 100B

### 2-iodobenzamidine

To a mixture of ammonium chloride (14 g) in toluene (200 mL) was added trimethylaluminum (131 mL, 2M mixture in toluene) in portions at 0° C. The mixture was stirred at 0° C. for 30 minutes. 2-Iodobenzonitrile (25 g) was added in one portion at 0° C. The mixture was stirred at 100° C. for 12 hours. The reaction mixture was cooled down to 0° C. and was quenched by addition of 200 mL of methanol. The resulting mixture was filtered. After filtering, the filtrate was concentrated under vacuum to provide the crude product which was precipitated from 500 mL of ethyl acetate to provide the title compound. ¹H NMR (400 MHz, dimethyl sulfoxide-d₆) δ ppm 9.47 (br s, 3H), 8.00 (m, 1H), 7.55 (m, 2H), 7.34 (ddd, J=7.88, 6.89, 2.21 Hz, 1H).

### Example 100C

### 4-(dimethoxymethyl)-2-(2-iodophenyl)pyrimidine

To a mixture of Example 100B (3.75 g) in methanol (30 mL) were added sodium methanolate (1.56 g) and Example 100A (2.51 g) in one portion at 25° C., and the mixture was stirred at 70° C. for 12 hours. The resulting mixture was concentrated under vacuum. The mixture was diluted with water (50 mL) and extracted with dichloromethane (2×50 mL). The combined organic layers were washed with brine (50 mL) and dried over Na₂SO₄. After filtering, the filtrate was concentrated under vacuum to provide the crude product which was purified by column chromatography on silica gel (petroleum ether:ethyl acetate=30:1 to 10:1) to provide the title compound. ¹H NMR (400 MHz, dimethyl sulfoxide-d₆) δ ppm 9.01 (d, J=5.26 Hz, 1H), 8.02 (dd, J=7.89, 0.66 Hz, 1H), 7.62 (m, 1H), 7.51-7.59 (m, 2H), 7.24 (d, J=1.53 Hz, 1H), 5.36 (s, 1H), 3.38 (s, 6H).

### Example 100D

### (2-(2-iodophenyl)pyrimidin-4-yl)methanol

To a mixture of Example 100C (3.75 g) in 1,4-dioxane (20 mL) was added 4M aqueous hydrochloric acid (20 mL) in one portion at 15° C. The mixture was stirred at 60° C. for 12 hours. The pH of the reaction mixture was adjusted to 8 by slow addition of 2M aqueous NaOH. NaBH₄ (0.79 g) was added to the reaction mixture in portions at 0° C. The reaction mixture was stirred at 0° C. for 2 hours. The resulting mixture was concentrated under vacuum. The mixture was diluted with water (15 mL) and extracted with dichloromethane (2×40 mL). The combined organic layers were washed with brine (40 mL), dried over Na₂SO₄ and filtered. The filtrate was concentrated under vacuum to provide the crude product which was washed with 15 mL of dichloromethane and 10 mL of methanol to provide the title compound. ¹H NMR (400 MHz, dimethyl sulfoxide-d₆) δ ppm 8.92 (d, J=5.07 Hz, 1H), 8.00 (dd, J=7.94, 0.88 Hz, 1H), 7.59-7.63 (m, 1H), 7.57 (d, J=5.29 Hz, 1H), 7.51 (td, J=7.50, 1.10 Hz, 1H), 7.21 (td, J=7.61, 1.76 Hz, 1H), 5.73 (t, J=5.95 Hz, 1H), 4.63 (d, J=5.95 Hz, 2H). MS (ESI) m/z 312.9 (M+H)⁺.

### Example 100E

### 2-(4-(hydroxymethyl)pyrimidin-2-yl)benzonitrile

To a suspension of Example 100D (156 mg), copper(I) iodide (9.52 mg), and potassium cyanide (65.1 mg) in degassed acetonitrile (1.25 mL) was added tetrakis(triphenylphosphine)palladium (0) (28.9 mg). The mixture was heated to reflux overnight. The reaction mixture was cooled to room temperature, diluted with ethyl acetate (10 mL) and filtered through diatomaceous earth. The filtrate was concentrated under vacuum and the residue was purified by silica gel chromatography on a CombiFlash® Teledyne Isco system eluting with 0-50% ethyl acetate in heptanes to provide the title compound. ¹H NMR (500 MHz, CDCl₃) δ ppm 8.85 (d, 1H), 8.54 (ddd, 1H), 7.88 (ddd, 1H), 7.75 (ddd, 1H), 7.61 (td, 1H), 7.28 (dt, 1H), 4.92 (dd, 2H), 3.77 (t, 1H). MS (ESI) m/z 212.0 (M+H)⁺.

### Example 100F

### 2-(4-(chloromethyl)pyrimidin-2-yl)benzonitrile

To a mixture of Example 100E (78 mg) and triphenylphosphine (126 mg) in dichloromethane (4 mL) cooled to 0° C. was added N-chlorosuccinimide (54.2 mg) in one portion. The mixture was warmed to room temperature and was was stirred for 1 hour. The mixture was directly loaded onto a silica gel column and purified using a CombiFlash® Teledyne Isco system eluting with 0-50% ethyl acetate in heptanes to provide the title compound. ¹H NMR (400 MHz, CDCl₃) δ ppm 8.96 (d, 1H), 8.41 (dd, 1H), 7.86 (dd, 1H), 7.73 (td, 1H), 7.65-7.53 (m, 2H), 4.75 (s, 2H). MS (ESI) m/z 230.0 (M+H)⁺.

### Example 100G

### ethyl (7R,20S)-18-chloro-10-{[2-(2-cyanophenyl)pyrimidin-4-yl]methoxy}-1-(4-fluorophenyl)-19-methyl-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylate

To a mixture of Example 100F (15.72 mg) and Example 65M (50 mg) in N,N-dimethylformamide (0.2 mL) was added cesium carbonate (66.9 mg). The mixture was stirred at room temperature for 2 hours. The reaction mixture was quenched with acetic acid (40 μL) and was diluted with 50% acetonitrile in water (2 mL). The mixture was purified by reverse-phase HPLC on a Gilson PLC 2020 using a Luna column (250×50 mm, 10 mm) (5-85% over 30 minutes with acetonitrile in water containing 0.1 trifluoroacetic acid) to provide the title compound after lyophilization. ¹H NMR (500 MHz, dimethyl sulfoxide-d₆) δ ppm 8.87 (d, 1H), 8.56 (s, 1H), 8.30 (dd, 1H), 8.01 (dd, 1H), 7.87 (td, 1H), 7.76 (td, 1H), 7.49 (d, 1H), 7.43 (d, 1H), 7.27 (d, 1H), 7.24-7.20 (m, 3H), 7.19-7.09 (m, 2H), 6.90 (d, 1H), 6.48 (d, 1H), 5.93 (dd, 1H), 5.26 (d, 1H), 5.09 (d, 1H), 4.34 (bs, 2H), 4.16 (bs, 2H), 4.11-4.00 (m, 2H), 3.22-3.10 (m, 2H), 3.04 (bs, 5H), 2.79 (s, 3H), 1.72 (s, 3H), 1.03 (t, 3H). MS (ESI) m/z 923.4 (M+H)⁺.

### Example 100H

### (7R,20S)-18-chloro-10-{[2-(2-cyanophenyl)pyrimidin-4-yl]methoxy}-1-(4-fluorophenyl)-19-methyl-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylic acid

To a mixture of Example 100G (29 mg) in methanol (0.3 mL) and tetrahydrofuran (0.3 mL) was added a mixture of lithium hydroxide (11.28 mg) in water (0.3 mL), and the reaction mixture was allowed to stir overnight. The reaction mixture was quenched with acetic acid (40 μL) and was diluted with methanol (2 mL). The mixture was purified by reverse-phase HPLC on a Gilson PLC 2020 using a Luna column (250×50 mm, 10 mm) (5-85% over 30 minutes with acetonitrile in water containing 0.1% trifluoroacetic acid) to provide the title compound after lyophilization. ¹H NMR (500 MHz, dimethyl sulfoxide-d₆) δ ppm 8.84 (d, 1H), 8.57 (s, 1H), 8.30 (dd, 1H), 8.00 (dd, 1H), 7.87 (td, 1H), 7.75 (td, 1H), 7.46 (d, 1H), 7.42 (d, 1H), 7.27 (d, 1H), 7.25-7.18 (m, 2H), 7.21-7.08 (m, 2H), 6.87 (d, 1H), 6.52-6.48 (m, 1H), 5.92 (dd, 1H), 5.26 (d, 1H), 5.07 (d, 1H), 4.28 (bs, 2H), 4.10 (bs, 2H), 3.28-3.21 (m, 1H), 3.18-3.12 (m, 1H), 3.02 (bs, 6H), 2.78 (s, 3H), 1.71 (s, 3H). MS (ESI) m/z 895.3 (M+H)⁺.

### Example 101

### (7R,20S)-18-chloro-10-({2-[2-(dimethylphosphoryl)phenyl]pyrimidin-4-yl}methoxy)-1-(4-fluorophenyl)-19-methyl-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylic acid

### Example 101A (2-(4-(hydroxymethyl)pyrimidin-2-yl)phenyl)dimethylphosphine oxide

To a suspension of Example 100D (312 mg), dimethylphosphine oxide (137 mg), Xantphos (4,5-bis(diphenylphosphino)-9,9-dimethylxanthene, 28.9 mg) and potassium phosphate tribasic (233 mg) in degassed N,N-dimethylformamide (2.5 mL) was added palladium(II) acetate (11.2 mg). The mixture was heated to 120° C. overnight. After cooling to room temperature, the mixture was diluted with ethyl acetate (10 mL) and filtered through diatomaceous earth. The filtrate was concentrated under vacuum and the residue was diluted with acetonitrile (3 mL) and purified by reverse-phase HPLC on a Gilson PLC 2020 using a Luna column (250×50 mm, 10 mm) (5-85% over 30 minutes with acetonitrile in water containing 0.1% trifluoroacetic acid) to provide the title compound after lyophilization. ¹H NMR (400 MHz, CDCl₃) δ ppm 8.79 (d, 1H), 8.20 (ddd, 1H), 8.07 (ddd, 1H), 7.67 (dtt, 2H), 7.36 (d, 1H), 4.84 (s, 2H), 1.88 (d, 6H). MS (ESI) m/z 263.1 (M+H)⁺.

### Example 101B

### (2-(2-(dimethylphosphoryl)phenyl)pyrimidin-4-yl)methyl methanesulfonate

To a mixture of Example 101A (44 mg) and triethylamine (0.070 mL) in dichloromethane (1.6 mL) cooled to 0° C. was added methanesulfonyl chloride (0.017 mL), and the mixture was stirred at 0° C. for 30 minutes. The reaction mixture was diluted with dichloromethane (10 mL) and was washed with brine (10 mL). The organic layer was dried over anhydrous sodium sulfate, filtered and concentrated under vacuum to provide the title compound which was used in the next step without further purification. LC/MS (APCI) m/z 340.4 (M+H)⁺.

### Example 101C

### ethyl (7R,20S)-18-chloro-10-({2-[2-(dimethylphosphoryl)phenyl]pyrimidin-4-yl}methoxy)-1-(4-fluorophenyl)-19-methyl-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylate

To a mixture of Example 101B (23.30 mg) and Example 65M (50 mg) in N,N-dimethylformamide (0.2 mL) was added cesium carbonate (66.9 mg). The mixture was stirred at room temperature for 1 hour. The reaction mixture was quenched with acetic acid (40 μL) and was diluted with 50% acetonitrile in water (2 mL). The mixture was purified by reverse-phase HPLC on a Gilson PLC 2020 using a Luna column (250×50 mm, 10 mm) (10-75% over 45 minutes with acetonitrile in water containing 0.1% trifluoroacetic acid) to provide the title compound after lyophilization. ¹H NMR (400 MHz, dimethyl sulfoxide-d₆) δ ppm 8.81 (d, 1H), 8.62 (s, 1H), 8.00 (ddd, 1H), 7.78 (ddd, 2H), 7.67 (pt, 2H), 7.52 (d, 1H), 7.34 (d, 1H), 7.33-7.18 (m, 4H), 7.21-7.11 (m, 2H), 6.87 (d, 1H), 6.43 (d, 1H), 5.95 (t, 1H), 5.20 (d, 1H), 5.12 (d, 1H), 4.35 (bs, 2H), 4.16 (bs, 2H), 4.14-3.98 (m, 2H), 3.19 (d, 2H), 3.05 (bs, 4H), 2.80 (s, 3H), 2.61 (bs, 1H), 1.80 (s, 3H), 1.68 (d, 3H), 1.65 (d, 3H), 1.03 (t, 3H). MS (ESI) m/z 974.2 (M+H)⁺.

### Example 101D

### (7R,20S)-18-chloro-10-({2-[2-(dimethylphosphoryl)phenyl]pyrimidin-4-yl}methoxy)-1-(4-fluorophenyl)-19-methyl-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylic acid

To a mixture of Example 101C (23 mg) in methanol (0.3 mL) and tetrahydrofuran (0.3 mL) was added a mixture of lithium hydroxide (8.48 mg) in water (0.3 mL), and the reaction mixture was allowed to stir overnight. The reaction mixture was quenched with acetic acid (30 μL) and was diluted with methanol (2 mL). The mixture was purified by reverse-phase HPLC on a Gilson PLC 2020 using a Luna column (250×50 mm, 10 mm) (5-85% over 30 minutes with acetonitrile in water containing 0.1% trifluoroacetic acid) to provide the title compound after lyophilization. ¹H NMR (500 MHz, dimethyl sulfoxide-d₆) δ ppm 8.78 (d, 1H), 8.62 (s, 1H), 7.99 (dd, 1H), 7.79 (dd, 1H), 7.73-7.62 (m, 2H), 7.53 (d, 1H), 7.32 (d, 1H), 7.29 (d, 1H), 7.29-7.19 (m, 3H), 7.20-7.06 (m, 2H), 6.87 (d, 1H), 6.44 (d, 1H), 5.91 (dd, 1H), 5.19 (d, 1H), 5.09 (d, 1H), 4.35 (d, 2H), 4.17 (bs, 2H), 3.19 (d, 1H), 3.03 (bs, 4H), 2.80 (s, 3H), 2.46 (bs, 1H), 1.79 (s, 3H), 1.69 (d, 3H), 1.67 (d, 3H). MS (ESI) m/z 946.2 (M+H)⁺.

### Example 102

### (7R,16R,21S)-19-chloro-1-(4-fluorophenyl)-16-({[2-(methanesulfonyl)ethyl](methyl)amino}methyl)-10-{[2-(2-methoxyphenyl)pyrimidin-4-yl]methoxy}-20-methyl-7,8,15,16-tetrahydro-18,21-etheno-13,9-(metheno)-6,14,17-trioxa-2-thia-3,5-diazacyclononadeca[1,2,3-cd]indene-7-carboxylic acid

The title compound was prepared using the conditions described in Example 82A and Example 82B substituting 2-(methylamino)-1-(methylsulfonyl)ethane for 1-[2-(methylsulfonyl)ethyl]piperazine. ¹H NMR (501 MHz, dimethyl sulfoxide-d₆) δ ppm 8.88 (d, J=5.2 Hz, 1H), 8.73 (s, 1H), 7.57-7.42 (m, 3H), 7.25-7.11 (m, 6H), 7.06 (td, J=7.5, 1.0 Hz, 1H), 6.96 (d, J=8.3 Hz, 1H), 6.91 (d, J=8.9 Hz, 1H), 6.82 (dd, J=9.0, 3.0 Hz, 1H), 6.11 (dd, J=5.3, 3.0 Hz, 1H), 5.65 (d, J=2.7 Hz, 1H), 5.24-5.07 (m, 2H), 4.57 (q, J=6.6 Hz, 1H), 4.45 (d, J=12.9 Hz, 1H), 4.35 (dd, J=13.2, 8.7 Hz, 1H), 3.86 (dd, J=16.8, 5.4 Hz, 1H), 3.77 (s, 3H), 2.98 (s, 3H), 3.30-3.20 (m, 1H) 2.94-2.76 (m, 4H), 2.68 (d, J=6.0 Hz, 2H), 2.22 (s, 6H). MS (ESI) m/z 940.1 (M+H)⁺.

### Example 103

### (7R,16R,21S)-19-chloro-16-[(dimethylamino)methyl]-1-(4-fluorophenyl)-10-{[2-(2-methoxyphenyl)pyrimidin-4-yl]methoxy}-20-methyl-7,8,15,16-tetrahydro-18,21-etheno-13,9-(metheno)-6,14,17-trioxa-2-thia-3,5-diazacyclononadeca[1,2,3-cd]indene-7-carboxylic acid

The title compound was prepared using the conditions described in Example 82A and Example 82B substituting dimethylamine hydrochloride for 1-[2-(methylsulfonyl)ethyl]piperazine. ¹H NMR (500 MHz, dimethyl sulfoxide-d₆) δ ppm 8.88 (d, J=5.1 Hz, 1H), 8.74 (s, 1H), 7.57-7.52 (m, 2H), 7.47 (ddd, J=8.4, 7.4, 1.8 Hz, 1H), 7.24-7.11 (m, 6H), 7.06 (td, J=7.5, 1.0 Hz, 1H), 6.93 (dd, J=19.5, 8.7 Hz, 2H), 6.81 (dd, J=9.0, 3.0 Hz, 1H), 6.10 (dd, J=5.3, 2.9 Hz, 1H), 5.63 (d, J=2.9 Hz, 1H), 5.29-5.05 (m, 2H), 4.55 (q, J=7.3 Hz, 1H), 4.45 (d, J=12.9 Hz, 1H), 4.32 (dd, J=13.2, 8.7 Hz, 1H), 3.87 (dd, J=16.8, 5.4 Hz, 1H), 3.77 (s, 3H), 2.87 (dd, J=17.2, 2.8 Hz, 1H), 2.59-2.52 (m, 2H) 2.24 (s, 3H), 2.16 (s, 6H). MS (ESI) m/z 848.3 (M+H)⁺.

### Example 104

### (7R,16R,21S)-19-chloro-10-{(R)-fluoro[2-(2-methoxyphenyl)pyrimidin-4-yl]methoxy}-1-(4-fluorophenyl)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid

### Example 104A

### ethyl (7R,16R,21S)-19-chloro-10-{fluoro[2-(2-methoxyphenyl)pyrimidin-4-yl]methoxy}-1-(4-fluorophenyl)-20-methyl-16-{[(4-methylbenzene-1-sulfonyl)oxy]methyl}-7,8,15,16-tetrahydro-18,21-etheno-9,13-(metheno)-6,14,17-trioxa-2-thia-3,5-diazacyclononadeca[1,2,3-cd]indene-7-carboxylate

To a mixture of Example 731 (100 mg) in acetonitrile (600 μL) was added N-fluorobenzenesulfonimide (80 mg) and the mixture was placed in a 55° C. pre-heated pi-block. The mixture was stirred at 55° C. for 18 hours and purification by preparative thin layer chromatography (20×20 cm; 0.5 mm thick; 75% ethyl acetate/heptane) provided the title compound. A 2.5:1 mixture of mono-fluorinated product at the benzylic position was obtained, and absolute configuration of minor and major was not determined. LC/MS (APCI) m/z 1021.2 (M+H)⁺.

### Example 104B

### ethyl (7R,16R,21S)-19-chloro-10-{fluoro[2-(2-methoxyphenyl)pyrimidin-4-yl]methoxy}-1-(4-fluorophenyl)-20-methyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylate

Example 104B was synthesized according to the procedure described for Example 73J, substituting Example 104A for Example 731. A 2.5:1 mixture of mono-fluorinated product at the benzylic position was obtained; absolute configuration of minor and major was not determined. LC/MS (APCI) m/z 949.2 (M+H)⁺.

### Example 104C

### (7R,16R,21S)-19-chloro-10-{(R)-fluoro[2-(2-methoxyphenyl)pyrimidin-4-yl]methoxy}-1-(4-fluorophenyl)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid

The title compound was synthesized as described in Example 82B, substituting Example 104B for Example 82A. Purification provided two diastereomers, the title compound and Example 105. Both were diastereomers of mono-fluorinated products. Absolute configuration was not determined and therefore the benzylic fluorine could read R or S. ¹H NMR (500 MHz, dimethyl sulfoxide-d₆) δ ppm 9.19 (d, J=5.1 Hz, 1H), 8.80 (s, 1H), 7.89 (d, J=5.1 Hz, 1H), 7.68 (dd, J=7.5, 1.8 Hz, 1H), 7.58 (td, J=8.1, 1.9 Hz, 1H), 7.35-7.23 (m, 6H), 7.21 (d, J=8.3 Hz, 1H), 7.16 (t, J=7.4 Hz, 1H), 7.04 (d, J=8.3 Hz, 1H), 7.00-6.83 (m, 2H), 6.23 (dd, J=5.0, 3.2 Hz, 1H), 5.89 (d, J=2.8 Hz, 1H), 4.72 (d, J=7.0 Hz, 1H), 4.66 (d, J=13.0 Hz, 1H), 4.43 (dd, J=13.2, 8.5 Hz, 1H), 3.87 (s, 4H), 3.00 (dd, J=17.6, 3.1 Hz, 1H), 2.76-2.62 (m, 2H), 2.57-2.38 (m, 8H), 2.28 (s, 3H), 2.24 (s, 3H). LC/MS (APCI) m/z 921.0 (M+H)⁺.

### Example 105

### (7R,16R,21S)-19-chloro-10-{(S)-fluoro[2-(2-methoxyphenyl)pyrimidin-4-yl]methoxy}-1-(4-fluorophenyl)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid

The title compound was isolated as a minor diastereomer during purification of Example 104C. The title compound and Example 104C are both diastereomers of mono-fluorinated products. Absolute configuration was not determined and therefore the benzylic fluorine could be R or S. ¹H NMR (500 MHz, dimethyl sulfoxide-d₆) δ ppm 9.10 (d, J=5.0 Hz, 1H), 8.69 (s, 1H), 7.81 (d, J=5.1 Hz, 1H), 7.62 (d, J=7.5 Hz, 1H), 7.50 (t, J=7.7 Hz, 1H), 7.29-7.15 (m, 6H), 7.13 (d, J=8.3 Hz, 1H), 7.08 (t, J=7.4 Hz, 1H), 6.94 (d, J=8.3 Hz, 1H), 6.88 (dd, J=9.1, 2.8 Hz, 1H), 6.77 (d, J=61.0 Hz, 1H), 6.14-6.04 (m, 1H), 5.74 (d, J=2.7 Hz, 1H), 4.66-4.49 (m, 2H), 4.35 (dd, J=13.2, 8.5 Hz, 1H), 3.83-3.71 (m, 4H), 2.84 (d, J=16.9 Hz, 1H), 2.67-2.53 (m, 2H), 2.48-2.32 (m, 8H), 2.22 (s, 3H), 2.18 (s, 3H). LC/MS (APCI) m/z 921.0 (M+H)⁺.

### Example 106

### (7R,16R,21S)-2,19-dichloro-1-(4-fluorophenyl)-10-{[2-(2-methoxyphenyl)pyrimidin-4-yl]methoxy}-20-methyl-16-[(4-methylpiperazin-1-yl)methyl]-7,8,15,16-tetrahydro-18,21-etheno-9,13-(metheno)-6,14,17-trioxa-2a,5-diazacyclononadeca[1,2,3-cd]indene-7-carboxylic acid

The title compound was isolated as a minor product during the synthesis and purification of Example 75D. ¹H NMR (400 MHz, dimethyl sulfoxide-d₆) δ ppm 8.84 (d, 1H), 7.88 (d, 1H), 7.63 (d, 1H), 7.55-7.49 (m, 1H), 7.48-7.39 (m, 1H), 7.27-7.08 (m, 6H), 7.07-6.91 (m, 2H), 6.83 (d, 1H), 6.73 (dd, 1H), 6.53 (d, 1H), 5.98 (d, 1H), 5.58 (dd, 1H), 5.27-5.00 (m, 3H), 4.32 (d, 1H), 4.03 (dd, 1H), 3.74 (s, 3H), 3.07 (br s, 6H), 2.92-2.81 (m, 2H), 2.78 (s, 3H), 2.64-2.50 (m, 2H), 2.44 (s, 3H). MS (ESI) m/z 919.3 (M+H)⁺.

### Example 107

### (7S,16R,21R)-2,19-dichloro-1-(4-fluorophenyl)-10-{[2-(2-methoxyphenyl)pyrimidin-4-yl]methoxy}-20-methyl-16-[(4-methylpiperazin-1-yl)methyl]-7,8,15,16-tetrahydro-18,21-etheno-9,13-(metheno)-6,14,17-trioxa-2a,5-diazacyclononadeca[1,2,3-cd]indene-7-carboxylic acid

The title compound was isolated as a minor product during the synthesis and purification of Example 75D. ¹H NMR (400 MHz, dimethyl sulfoxide-d₆) δ ppm 8.85 (d, 1H), 7.93 (d, 1H), 7.60 (d, 1H), 7.54-7.49 (m, 1H), 7.48-7.40 (m, 1H), 7.26 (d, 1H), 7.19-7.09 (m, 6H), 7.07-7.00 (m, 2H), 6.88 (d, 1H), 6.83 (d, 1H), 6.70 (dd, 1H), 6.61 (d, 1H), 5.88 (d, 1H), 5.68 (dd, 1H), 5.23-5.08 (m, 3H), 4.84 (br s, 2H), 4.19-4.11 (m, 2H), 3.76 (s, 3H), 3.05 (br s, 4H), 2.92-2.81 (m, 3H), 2.78 (s, 3H), 2.69-2.50 (m, 2H), 2.40 (s, 3H). MS (ESI) m/z 919.2 (M+H)⁺.

### Example 108

### (7R,16R,21S)-19-chloro-1-cyclopropyl-10-{[2-(2-methoxyphenyl)pyrimidin-4-yl]methoxy}-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid

### Example 108A

### 5-bromo-4-chloro-6-cyclopropylthieno[2,3-d]pyrimidine

A mixture of Example 1C (520 mg), cyclopropylboronic acid (178 mg), potassium phosphate tribasic (882 mg), tricyclohexylphosphine (38 mg) and palladium (II) acetate (15 mg) in a 100 mL flask was sparged with argon for 10 minutes, and toluene (10 mL) and water (2 mL) were added. The reaction mixture was heated at 100° C. for 24 hours, cooled and filtered. The filtrate was concentrated. The residue was purified by flash chromatography, and was eluted with 0.5% ethyl acetate in heptanes to provide the title compound. MS (APCI) m/z 291.0 (M+H)⁺.

### Example 108B

### (R)-ethyl 2-((5-bromo-6-cyclopropylthieno[2,3-d]pyrimidin-4-yl)oxy)-3-(5-((tert-butyldimethylsilyl)oxy)-2-((2-(2-methoxyphenyl)pyrimidin-4-yl)methoxy)phenyl)propanoate

To a mixture of Example 108A (1.055 g) and Example 68B (1.635 g) in N,N-dimethylformamide (10 mL) was added cesium carbonate (1.978 g) and tert-butanol (10 mL). The mixture was stirred at ambient temperature overnight, diluted with ethyl acetate and washed with water and brine. The organic layer was dried over Na₂SO₄, filtered, and concentrated. The residue was purified by flash chromatography, eluting with 0-50% ethyl acetate in heptanes to provide the title compound. MS (APCI) m/z 793.1 (M+H)⁺.

### Example 108C

### (2R)-ethyl 2-((5-((1S)-4-(((R)-1-(bis(4-methoxyphenyl)(phenyl)methoxy)-3-(tosyloxy)propan-2-yl)oxy)-3-chloro-2-methylphenyl)-6-cyclopropylthieno[2,3-d]pyrimidin-4-yl)oxy)-3-(5-((tert-butyldimethylsilyl)oxy)-2-((2-(2-methoxyphenyl)pyrimidin-4-yl)methoxy)phenyl)propanoate

To a mixture of Example 108B (0.991 g), Example 73D (1 g) and Pd(amphos)Cl₂ (bis(di-tert-butyl(4-dimethylaminophenyl)phosphine)dichloropalladium(II), 0.133 g) was added a mixture of potassium phosphate (0.797 g) in tetrahydrofuran (25 mL) and water (5 mL). The mixture was sparged with nitrogen for 10 minutes, stirred at ambient temperature overnight, diluted with ethyl acetate, and washed with water and brine. The organic layer was dried over Na₂SO₄, filtered, and concentrated. The residue was purified by flash chromatography, eluting with 0-66% ethyl acetate in heptanes to provide the title compound. MS (ESI) m/z 1384.5 (M+H)⁺.

### Example 108D

### (2R)-ethyl 2-((5-((1S)-4-(((R)-1-(bis(4-methoxyphenyl)(phenyl)methoxy)-3-(tosyloxy)propan-2-yl)oxy)-3-chloro-2-methylphenyl)-6-cyclopropylthieno[2,3-d]pyrimidin-4-yl)oxy)-3-(5-hydroxy-2-((2-(2-methoxyphenyl)pyrimidin-4-yl)methoxy)phenyl)propanoate

Example 108C (1.39 g) in CH₂Cl₂ (10 mL), cooled in an ice bath, was treated with 1 M tetrabutyl ammonium fluoride in tetrahydrofuran (1.306 mL) for 10 minutes. The mixture was directly loaded onto a silica gel column, and was eluted with 0-70% ethyl acetate in heptanes to provide the title compound. MS (ESI) m/z 1270.4 (M+H)⁺.

### Example 108E

### ethyl (7R,16R,21S)-16-{[bis(4-methoxyphenyl)(phenyl)methoxy]methyl}-19-chloro-1-cyclopropyl-10-{[2-(2-methoxyphenyl)pyrimidin-4-yl]methoxy}-20-methyl-7,8,15,16-tetrahydro-18,21-etheno-9,13-(metheno)-6,14,17-trioxa-2-thia-3,5-diazacyclononadeca[1,2,3-cd]indene-7-carboxylate

To a mixture of Example 108D (1.15 g) in N,N-dimethylformamide (80 mL) was added cesium carbonate (1.475 g). The mixture was stirred at ambient temperature for 2 days, diluted with ethyl acetate and washed with water and brine. The organic layer was dried over Na₂SO₄, filtered, and concentrated. The residue was purified by flash chromatography, eluting with 0-66% ethyl acetate in heptanes to provide the title compound. MS (ESI) m/z 1097.5 (M+H)⁺.

### Example 108F

### ethyl (7R,16R,21S)-19-chloro-1-cyclopropyl-16-(hydroxymethyl)-10-{[2-(2-methoxyphenyl)pyrimidin-4-yl]methoxy}-20-methyl-7,8,15,16-tetrahydro-18,21-etheno-9,13-(metheno)-6,14,17-trioxa-2-thia-3,5-diazacyclononadeca[1,2,3-cd]indene-7-carboxylate

To a mixture of Example 108E (0.82 g) in CH₂Cl₂ (4 mL) and methanol (4 mL) was added formic acid (3.67 mL). The mixture was stirred at ambient temperature for 10 minutes, diluted with ethyl acetate and washed with water and brine. The organic layer was dried over Na₂SO₄, filtered, and concentrated. The residue was purified by flash chromatography, eluting with 0-70% ethyl acetate in heptanes to provide the title compound. MS (ESI) m/z 795.4 (M+H)⁺.

### Example 108G

### ethyl (7R,16R,21S)-19-chloro-1-cyclopropyl-10-{[2-(2-methoxyphenyl)pyrimidin-4-yl]methoxy}-20-methyl-16-{[(4-methylbenzene-1-sulfonyl)oxy]methyl}-7,8,15,16-tetrahydro-18,21-etheno-9,13-(metheno)-6,14,17-trioxa-2-thia-3,5-diazacyclononadeca[1,2,3-cd]indene-7-carboxylate

To a mixture of Example 108F (267 mg) in CH₂Cl₂ (4 mL) was added triethylamine (0.140 mL) and p-toluenesulfonyl chloride (128 mg). The mixture was stirred at ambient temperature for 22 hours and was directly loaded onto a 60 g silica gel cartridge, eluting with 0-70% ethyl acetate in heptanes to provide the title compound. MS (ESI) m/z 949.4 (M+H)⁺.

### Example 108H

### ethyl (7R,16R,21S)-19-chloro-1-cyclopropyl-10-{[2-(2-methoxyphenyl)pyrimidin-4-yl]methoxy}-20-methyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylate

To a mixture of Example 108G (280 mg) in N,N-dimethylformamide (1 mL) was added 1-methylpiperazine (1.079 mL). The mixture was stirred at ambient temperature for 24 hours at 40° C., diluted with ethyl acetate, and washed with water and brine. The organic layer was dried over Na₂SO₄, filtered, and concentrated to provide the title compound. MS (ESI) m/z 877.2 (M+H)⁺.

### Example 108I

### (7R,16R,21S)-19-chloro-1-cyclopropyl-10-{[2-(2-methoxyphenyl)pyrimidin-4-yl]methoxy}-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid

Example 108H (280 mg) in tetrahydrofuran (5 mL) was cooled in an ice bath for 20 minutes and a cold mixture of 1 M aqueous LiOH (5.74 mL) and methanol (5 mL) was added. The mixture was stirred at ambient temperature for 2.5 days, and the reaction mixture was quenched with acetic acid (0.913 mL). The resulting mixture was concentrated. The residue was purified by RP HPLC on a Gilson PLC 2020 using a Luna column (250×50 mm, 10 mm), eluting with 30%-45% acetonitrile in 0.1% trifluoroacetic acid water to provide the title compound. ¹H NMR (400 MHz, dimethyl sulfoxide-d₆) δ ppm 12.70 (s, br, 1H), 9.42 (s, br, 1H), 8.88 (d, 1H), 8.63 (s, 1H), 7.59-7.44 (m, 3H), 7.34 (d, 1H), 7.24 (d, 1H), 7.16 (d, 1H), 7.06 (t, 1H), 6.91 (d, 1H), 6.84 (dd, 1H), 6.11 (dd, 1H), 5.70 (d, 1H), 5.17 (q, 2H), 4.61 (d, 1H), 4.50 (d, 1H), 4.42 (dd, 1H), 3.83 (dd, 1H), 3.77 (s, 3H), 3.17-2.70 (m, 10H), 2.12 (s, 3H), 1.75 (tt, 1H), 0.99 (ttd, 2H), 0.85-0.75 (m, 1H), 0.75-0.64 (m, 1H). MS (APCI) m/z 850.3 (M+H)⁺.

### Example 109

### (7S,16R,21S)-19-chloro-1-cyclopropyl-10-{[2-(2-methoxyphenyl)pyrimidin-4-yl]methoxy}-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid

The title compound was isolated as a minor product during the synthesis and purification of Example 108H. ¹H NMR (500 MHz, dimethyl sulfoxide-d₆) δ ppm 9.48 (s, 1H), 8.85 (d, 1H), 8.55 (s, 1H), 7.60 (d, 1H), 7.50 (dd, 1H), 7.46-7.41 (m, 1H), 7.15-7.10 (m, 2H), 7.05-6.98 (m, 2H), 6.91 (d, 1H), 6.77 (dd, 1H), 5.87 (d, 1H), 5.74 (dd, 1H), 5.26-5.11 (m, 2H), 4.89 (m, 1H), 4.28 (dd, 1H), 4.20 (dd, 1H), 3.74 (s, 3H), 3.42-3.33 (m, 3H), 3.24-2.76 (m, 10H), 2.33 (s, 3H), 1.77-1.68 (m, 1H), 0.97 (dddd, 2H), 0.82-0.72 (m, 1H), 0.73-0.65 (m, 1H). MS (APCI) m/z 850.3 (M+H)⁺.

### Example 110

### (7R,16R,21R)-23-chloro-1-cyclopropyl-10-{[2-(2-methoxyphenyl)pyrimidin-4-yl]methoxy}-22-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid

The title compound was isolated as a minor product during the synthesis and purification of Example 108H. ¹H NMR (400 MHz, dimethyl sulfoxide-d₆) δ ppm 13.21 (s, br, 1H), 9.47 (s, br, 1H), 8.83 (d, 1H), 8.50 (s, 1H), 7.62 (d, 1H), 7.50 (dd, 1H), 7.44 (ddd, 1H), 7.21 (d, 1H), 7.13 (d, 1H), 7.02 (td, 1H), 6.95 (d, 1H), 6.85 (d, 1H), 6.78 (dd, 1H), 6.03 (d, 1H), 5.70 (dd, 1H), 5.17 (q, 4H), 4.43 (d, 1H), 4.10 (dd, 1H), 3.74 (s, 3H), 3.43 (m, 2H), 3.28 (m, 2H), 3.08 (m, 2H), 2.91 (m, 2H), 2.80 (s, 3H), 2.58-2.52 (m, 2H), 2.30 (s, 3H), 1.88 (tt, 1H), 0.99 (tdd, 2H), 0.83-0.66 (m, 2H). LC/MS (APCI) m/z 850.6 (M+H)⁺.

### Example 111

### (7R,16R)-19-chloro-1-(4-fluorophenyl)-10-{[2-(2-methoxyphenyl)pyrimidin-4-yl]methoxy}-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic acid

### Example 111A

### (R)-3-(bis(4-methoxyphenyl)(phenyl)methoxy)-2-(4-bromo-2-chlorophenoxy)propyl 4-methylbenzenesulfonate

To a mixture of Example 73B (411 mg) and 4-bromo-2-chlorophenol (202 mg) in tetrahydrofuran (7.5 mL) was added triphenylphosphine (393 mg) and di-tert-butyl azodicarboxylate (345 mg), and the reaction mixture was warmed to 45° C. for 3 hours. The reaction mixture was cooled, diluted with ethyl acetate, filtered and concentrated. The residue was purified by normal phase MPLC on a Teledyne Isco Combiflash Rf+ (5-90% ethyl acetate in heptanes) to provide the title compound. ¹H NMR (400 MHz, dimethyl sulfoxide-d₆) δ ppm 7.72-7.63 (m 3H), 7.41-7.35 (m, 3H), 7.31-7.08 (m, 9H), 7.00 (d, 1H), 6.90-6.78 (m, 4H), 4.86-4.76 (m, 1H), 4.33-4.23 (m, 2H), 3.76-3.69 (m, 6H), 3.23-3.13 (m, 2H), 2.37 (s, 3H).

### Example 111B

### (R)-3-(bis(4-methoxyphenyl)(phenyl)methoxy)-2-(2-chloro-4-(4,4,5,5-tetramethyl-1,3,2-dioxaborolan-2-yl)phenoxy)propyl 4-methylbenzenesulfonate

To a vial containing Example 111A (324 mg), potassium acetate (86 mg, heated at 100° C. under vacuum for at least one hour), 1,1′-bis(diphenylphosphino)ferrocene-palladium(II)dichloride dichloromethane complex (17.92 mg), and bis(pinacolato)diboron (134 mg) was added 2-methyl tetrahydrofuran (2.2 mL). The mixture was purged with nitrogen and heated at 90° C. overnight. The reaction mixture was diluted with ethyl acetate, filtered over diatomaceous earth, and concentrated. The crude residue was purified by normal phase MPLC on a Teledyne Isco Combiflash Rf+ (5-90% ethyl acetate in heptanes) to provide the title compound. ¹H NMR (400 MHz, dimethyl sulfoxide-d₆) δ ppm 7.69 (d, 2H), 7.58 (d, 1H), 7.46 (dd, 1H), 7.36 (d, 2H), 7.29-7.08 (m, 9H), 7.01 (d, 1H), 6.87-6.76 (m, 4H), 4.92-4.81 (m, 1H), 4.35-4.23 (m, 2H), 3.77-3.66 (m, 6H), 3.25-3.14 (m, 2H), 2.35 (s, 3H), 1.29 (s, 12H).

### Example 111C

### (R)-ethyl 2-((5-(4-(((R)-1-(bis(4-methoxyphenyl)(phenyl)methoxy)-3-(tosyloxy)propan-2-yl)oxy)-3-chlorophenyl)-6-(4-fluorophenyl)thieno[2,3-d]pyrimidin-4-yl)oxy)-3-(5-((tert-butyldimethylsilyl)oxy)-2-((2-(2-methoxyphenyl)pyrimidin-4-yl)methoxy)phenyl)propanoate

A vial containing Example 111B (197 mg), Example 68C (163 mg), cesium carbonate (188 mg) and bis(di-tert-butyl(4-dimethylaminophenyl)phosphine)dichloropalladium(II) (13.65 mg) was evacuated and backfilled with nitrogen several times. To the vial was added degassed tetrahydrofuran (1.5 mL) and water (385 μL), and the reaction mixture was stirred overnight at room temperature. 1-Pyrrolidinecarbodithioic acid ammonium salt (3.2 mg) was added, and the reaction mixture was allowed to stir for 30 minutes. The reaction mixture was diluted with ethyl acetate and filtered over diatomaceous earth. Brine and water were added, and the aqueous layer was extracted with ethyl acetate three times. The combined organic layers were dried over anhydrous sodium sulfate, filtered and concentrated. The crude residue was purified by normal phase MPLC on a Teledyne Isco Combiflash Rf+ (5-65% ethyl acetate in heptanes) to provide the title compound. MS (ESI) m/z 1423.8 (M+H)⁺.

### Example 111D

### (R)-ethyl 2-((5-(4-(((R)-1-(bis(4-methoxyphenyl)(phenyl)methoxy)-3-(tosyloxy)propan-2-yl)oxy)-3-chlorophenyl)-6-(4-fluorophenyl)thieno[2,3-d]pyrimidin-4-yl)oxy)-3-(5-hydroxy-2-((2-(2-methoxyphenyl)pyrimidin-4-yl)methoxy)phenyl)propanoate

To a mixture of Example 111C (230 mg) in tetrahydrofuran (1.6 mL) was added tetrabutylammonium fluoride (162 μL, 1 M in tetrahydrofuran), and the reaction mixture was allowed to stir. After 20 minutes, the reaction mixture was quenched with saturated aqueous ammonium chloride and was extracted with ethyl acetate three times. The combined organic layers were dried over anhydrous sodium sulfate, filtered and concentrated. The crude residue was purified by normal phase MPLC on a Teledyne Isco Combiflash Rf+ (15-75% ethyl acetate in heptanes) to provide the title compound. MS (ESI) m/z 1311.6 (M+H)⁺.

### Example 111E

### ethyl (7R,16S)-16-{[bis(4-methoxyphenyl)(phenyl)methoxy]methyl}-19-chloro-1-(4-fluorophenyl)-10-{[2-(2-methoxyphenyl)pyrimidin-4-yl]methoxy}-7,8,15,16-tetrahydro-18,21-etheno-9,13-(metheno)-6,14,17-trioxa-2-thia-3,5-diazacyclononadeca[1,2,3-cd]indene-7-carboxylate

A mixture of Example 111D (176 mg) and cesium carbonate (219 mg) in N,N-dimethyl formamide (13.4 mL) was stirred at room temperature for 22 hours. The reaction mixture was transferred to a separatory funnel with water and ethyl acetate. The aqueous layer was extracted with ethyl acetate three times. The combined organic layers were dried over anhydrous sodium sulfate, filtered and concentrated. The crude residue was purified by normal phase MPLC on a Teledyne Isco Combiflash Rf+ (10-75% ethyl acetate in heptanes) to provide the title compound. MS (ESI) m/z 1137.4 (M+H)⁺.

### Example 111F

### ethyl (7R,16R)-19-chloro-1-(4-fluorophenyl)-16-(hydroxymethyl)-10-{[2-(2-methoxyphenyl)pyrimidin-4-yl]methoxy}-7,8,15,16-tetrahydro-18,21-etheno-9,13-(metheno)-6,14,17-trioxa-2-thia-3,5-diazacyclononadeca[1,2,3-cd]indene-7-carboxylate

To a mixture of Example 111E (119 mg) in dichloromethane (530 μL) and methanol (530 μL) was added formic acid (520 μL), and the reaction mixture was allowed to stir. After 30 minutes, the reaction mixture was quenched slowly with saturated aqueous sodium bicarbonate and was extracted with ethyl acetate three times. The combined organics extracts were dried over anhydrous sodium sulfate, filtered and concentrated. The residue was purified by normal phase MPLC on a Teledyne Isco Combiflash Rf+ (15-90% ethyl acetate in heptanes) to provide the title compound. MS (ESI) m/z 835.2 (M+H)⁺.

### Example 111G

### ethyl (7R,16S)-19-chloro-1-(4-fluorophenyl)-10-{[2-(2-methoxyphenyl)pyrimidin-4-yl]methoxy}-16-{[(4-methylbenzene-1-sulfonyl)oxy]methyl}-7,8,15,16-tetrahydro-18,21-etheno-9,13-(metheno)-6,14,17-trioxa-2-thia-3,5-diazacyclononadeca[1,2,3-cd]indene-7-carboxylate

To a mixture of Example 111F (77 mg) and triethylamine (64 μL) in dichloromethane (900 μL) was added p-toluenesulfonyl chloride (52.7 mg), and the reaction mixture was stirred. After 4 hours, the reaction mixture was diluted with dichloromethane and water. The aqueous layer was extracted with dichloromethane three times, and the combined organic layers were dried over anhydrous sodium sulfate, filtered, and concentrated. The crude residue was purified by normal phase MPLC on a Teledyne Isco Combiflash Rf+ (10-75% ethyl acetate in heptanes) to provide the title compound. MS (ESI) m/z 989.4 (M+H)⁺.

### Example 111H

### ethyl (7R,16R)-19-chloro-1-(4-fluorophenyl)-10-{[2-(2-methoxyphenyl)pyrimidin-4-yl]methoxy}-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylate

A mixture of Example 111G (84 mg) and 1-methylpiperazine (255 μL) in N,N-dimethyl formamide (280 μL) was stirred at 40° C. overnight. The reaction mixture was cooled, taken up in dimethyl sulfoxide (600 μL) and purified by RP-HPLC on a Gilson PLC 2020 using a Luna column (250×50 mm, 10 mm) (5-80% over 30 minutes with acetonitrile in water containing 0.1% trifluoroacetic acid) to provide the title compound after lyophilization. MS (ESI) m/z 917.3 (M+H)⁺.

### Example 111I

### (7R,16R)-19-chloro-1-(4-fluorophenyl)-10-{[2-(2-methoxyphenyl)pyrimidin-4-yl]methoxy}-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylic acid

To a mixture of Example 111H (36 mg) in tetrahydrofuran (440 μL) and methanol (440 μL) at 0° C. was added a mixture of lithium hydroxide (18.8 mg) in water (440 μL), and the reaction mixture was allowed to stand at 0° C. overnight. The reaction mixture was quenched with trifluoroacetic acid (73 μL), taken up in dimethyl sulfoxide and purified by RP-HPLC on a Gilson PLC 2020 using a Luna column (250×50 mm, 10 mm) (5-65% over 30 minutes with acetonitrile in water containing 0.1% trifluoroacetic acid) to provide the title compound after lyophilization. ¹H NMR (500 MHz, dimethyl sulfoxide-d₆) δ ppm 8.82 (d, 1H), 8.61 (s, 1H), 7.64 (d, 1H), 7.53 (d, 1H), 7.49 (dd, 1H), 7.46-7.40 (m, 1H), 7.37-7.29 (m, 2H), 7.24-7.08 (m, 4H), 7.06-6.97 (m, 1H), 6.80 (d, 1H), 6.74-6.66 (m, 2H), 6.14 (d, 1H), 5.99 (dd, 1H), 5.20-5.06 (m, 3H), 4.35 (d, 1H), 3.72 (s, 3H), 3.52-3.00 (m, 9H), 2.99-2.83 (m, 4H), 2.79 (s, 3H), 2.72-2.54 (m, 2H).

### Example 112

### (7R,16R)-23-chloro-1-(4-fluorophenyl)-10-{[2-(2-methoxyphenyl)pyrimidin-4-yl]methoxy}-16-[(4-methylpiperazin-1-yl)methyl]-7,8,15,16-tetrahydro-18,21-etheno-9,13-(metheno)-2,6,14,17-tetraoxa-3,5-diazacyclononadeca[1,2,3-cd]indene-7-carboxylic acid

### Example 112A

### (R)-ethyl 2-((5-bromo-6-(4-fluorophenyl)furo[2,3-d]pyrimidin-4-yl)oxy)-3-(5-((tert-butyldimethylsilyl)oxy)-2-((2-(2-methoxyphenyl)pyrimidin-4-yl)methoxy)phenyl)propanoate

A mixture of Example 49C (283 mg), Example 68B (465 mg) and cesium carbonate (844 mg) in anhydrous tert-butanol (10 mL) was heated to 70° C. for 5 hours followed by stirring overnight at room temperature. The solvent was reduced in vacuo, water was added, and the mixture was extracted twice with dichloromethane. The combined organic layers were washed with water and brine, dried over MgSO₄, filtered, and concentrated in vacuo. The residue obtained was purified by silica gel flash chromatography (40 g Grace Reveleris column, eluting with 2-75% ethyl acetate in heptane) to provide the title compound. MS (ESI) m/z 829.2 (M+H)⁺.

### Example 112B

### (R)-3-(bis(4-methoxyphenyl)(phenyl)methoxy)-2-hydroxypropyl 4-methylbenzenesulfonate

The title compound was prepared in the same manner as its enantiomer, Example 73B, using the conditions described in Example 73A and Example 73B, and starting with (R)-(2,2-dimethyl-1,3-dioxolan-4-yl)methyl 4-methylbenzenesulfonate.

### Example 112C

### (S)-3-(bis(4-methoxyphenyl)(phenyl)methoxy)-2-(4-bromo-2-chlorophenoxy)propyl 4-methylbenzenesulfonate

Example 112B (100 mg), 4-bromo-2-chlorophenol (45.4 mg) and triphenylphosphine (71.7 mg) were mixed under argon. Tetrahydrofuran (6 mL) was added, followed by trimethylamine (25 μL), and di-tert-butyl azodicarboxylate (63.0 mg). The reaction mixture was stirred overnight at room temperature. The solvent was removed in vacuo and the residue was purified by silica gel flash chromatography (4 g Silica RediSep® Rf Gold Teledyne Isco column, eluting with 0-30% ethyl acetate in cyclohexane) to provide the title compound which was directly used in the next step.

### Example 112D

### (R)-1-(3-(bis(4-methoxyphenyl)(phenyl)methoxy)-2-(4-bromo-2-chlorophenoxy)propyl)-4-methylpiperazine

A mixture of Example 112C (121.8 mg, 60% purity), 1-methylpiperazine (92 μL) and triethylamine (69 μL) in N,N-dimethylformamide (4 mL) was heated to 80° C. overnight. Water was added and the mixture was extracted with ethyl acetate. The combined organic layers were washed with water, dried over MgSO₄, filtered, and concentrated in vacuo. The residue obtained was purified by silica gel flash chromatography (4 g Silica RediSep® Rf Gold Teledyne Isco column, eluting with 0-30% methanol in dichloromethane) to provide the title compound. MS (ESI) m/z 365.2 ([M−DMTrt]+H)⁺.

### Example 112E

### (R)-1-(3-(bis(4-methoxyphenyl)(phenyl)methoxy)-2-(2-chloro-4-(4,4,5,5-tetramethyl-1,3,2-dioxaborolan-2-yl)phenoxy)propyl)-4-methylpiperazine

Example 112D (204 mg), potassium acetate (60.1 mg), 1,1′-bis(diphenylphosphino)ferrocene-palladium (II) dichloride dichloromethane complex (12.5 mgl) and bis(pinacolato)diboron (86 mg) was added to a reaction vial. The mixture was degassed with argon. 2-Methyltetrahydrofuran (3 mL) was added and the reaction mixture was heated for 12 hours at 90° C. The solvent was removed in vacuo and the crude material was purified by silica gel flash chromatography (4 g Silica RediSep® Rf Gold Teledyne Isco column, eluting with 0-40% methanol in dichloromethane) to provide the title compound. MS (ESI) m/z 411.4 ([M−DMTr]+2H)⁺.

### Example 112F

### (R)-ethyl 2-((5-(4-(((S)-1-(bis(4-methoxyphenyl)(phenyl)methoxy)-3-(4-methylpiperazin-1-yl)propan-2-yl)oxy)-3-chlorophenyl)-6-(4-fluorophenyl)furo[2,3-d]pyrimidin-4-yl)oxy)-3-(5-((tert-butyldimethylsilyl)oxy)-2-((2-(2-methoxyphenyl)pyrimidin-4-yl)methoxy)phenyl)propanoate

A mixture of Example 112A (150 mg), Example 112E (161 mg), cesium carbonate (177.0 mg) and bis (di-tert-butyl(4-dimethylaminophenyl)phosphine)dichloropalladium (II) (12.8 mg) were stirred under argon. A mixture of tetrahydrofuran (4 mL) and water (1 mL) was degassed and added. After stirring for 48 hours at room temperature, water was added and the mixture was extracted twice with ethyl acetate. The combined organic layers were washed with water, dried over MgSO₄, filtered, and concentrated in vacuo. The crude product was used without further purification in the next step. MS (ESI) m/z 1033.3 ([M−DMTr]+H)⁺.

### Example 112G

### (R)-ethyl 3-(5-((tert-butyldimethylsilyl)oxy)-2-((2-(2-methoxyphenyl)pyrimidin-4-yl)methoxy)phenyl)-2-((5-(3-chloro-4-(((S)-1-hydroxy-3-(4-methylpiperazin-1-yl)propan-2-yl)oxy)phenyl)-6-(4-fluorophenyl)furo[2,3-d]pyrimidin-4-yl)oxy)propanoate

Formic acid (920 mg) was added to a mixture of Example 112F (267 mg) in dichloromethane/methanol (2.5 mL/2.5 mL) and the reaction mixture was stirred overnight at room temperature. The pH was adjusted to 9 under ice-cooling using saturated aqueous NaHCO₃. After extraction three times with dichloromethane, the combined organic layers were washed with water, dried over MgSO₄, filtered, and concentrated in vacuo. The residue obtained was purified by silica gel flash chromatography (4 g Silica RediSep® Rf Gold Teledyne Isco, eluting with 0-30% methanol in dichloromethane) to provide the title compound. MS (ESI) m/z 1033.3 (M+H)⁺.

### Example 112H

### (R)-ethyl 2-((5-(3-chloro-4-(((S)-1-hydroxy-3-(4-methylpiperazin-1-yl)propan-2-yl)oxy)phenyl)-6-(4-fluorophenyl)furo[2,3-d]pyrimidin-4-yl)oxy)-3-(5-hydroxy-2-((2-(2-methoxyphenyl)pyrimidin-4-yl)methoxy)phenyl)propanoate

Tetrabutyl ammonium fluoride (0.371 mL, 1M mixture in tetrahydrofuran) was added to a mixture of Example 112G (128 mg) in tetrahydrofuran (5 mL). After stirring for 1 hour at room temperature, aqueous ammonium chloride mixture (10%) was added, and the mixture was extracted twice with ethyl acetate. The combined extracts were washed with water, dried over MgSO₄, and filtered. The solvent was reduced in vacuo. The residue obtained was purified by silica gel flash chromatography (4 g Silica RediSep® Rf Gold Teledyne Isco, column, eluting with 0-30% methanol in dichloromethane) to provide the title compound. MS (ESI) m/z 919.3 (M+H)⁺.

### Example 112I

### ethyl (7R,16R)-19-chloro-1-(4-fluorophenyl)-10-{[2-(2-methoxyphenyl)pyrimidin-4-yl]methoxy}-16-[(4-methylpiperazin-1-yl)methyl]-7,8,15,16-tetrahydro-18,21-etheno-9,13-(metheno)-2,6,14,17-tetraoxa-3,5-diazacyclononadeca[1,2,3-cd]indene-7-carboxylate

Example 112H (57.0 mg) and triphenylphosphine (48.8 mg) were mixed in a microwave vial under an argon atmosphere. Dry and degassed tetrahydrofuran (4 mL) was added. Di-tert-butyl azodicarboxylate (32.0 mg) was added in one portion. After stirring overnight at room temperature, water was added and the mixture was extracted with twice ethyl acetate. The combined extracts were dried over MgSO₄, and filtered. The solvent was reduced in vacuo. To the residue, dichloromethane was added and the precipitate was filtered off. The organic layer was reduced in vacuo and the crude material was purified by silica gel flash chromatography (4 g Silica RediSep® Rf Gold Teledyne Isco column, eluting with 1-100% ethyl acetate in heptane, and then with 100% methanol) to provide the title compound. MS (ESI) m/z 901.3 (M+H)⁺.

### Example 112J

### (7R,16R)-23-chloro-1-(4-fluorophenyl)-10-{[2-(2-methoxyphenyl)pyrimidin-4-yl]methoxy}-16-[(4-methylpiperazin-1-yl)methyl]-7,8,15,16-tetrahydro-18,21-etheno-9,13-(metheno)-2,6,14,17-tetraoxa-3,5-diazacyclononadeca[1,2,3-cd]indene-7-carboxylic acid

LiOH (17.0 mg) was added to a mixture of Example 112I (32 mg) in methanol/tetrahydrofuran/water (0.4 mL/0.4 mL/0.4 mL). The reaction mixture was stirred overnight at room temperature. The solvents were reduced in vacuo. The residue was dissolved in tetrahydrofuran/water (1.0 mL/0.5 mL) and subsequently LiOH (17.0 mg) was added. The reaction mixture was stirred overnight at room temperature. The solvent was removed in vacuo. Purification by HPLC (Waters X-Bridge C18 19×150 mm 5 μm column, gradient 5-100% acetonitrile+0.1% trifluoroacetic acid in water+0.1% trifluoroacetic acid) provided the title compound. ¹H NMR (400 MHz, dimethyl sulfoxide-d₆) δ ppm 13.28 (s, 1H), 9.37 (bs, 1H), 8.87 (d, 1H), 8.56 (s, 1H), 7.65 (m, 1H), 7.60-7.55 (m, 3H), 7.51 (m, 1H), 7.45 (m, 1H), 7.31-7.26 (m, 3H), 7.17-7.13 (m, 2H), 7.04 (m, 1H), 6.86 (m, 1H), 6.76 (m, 1H), 6.27 (s, 1H), 5.88 (bs, 1H), 5.20-5.15 (m, 2H), 5.07 (bs, 1H), 4.30 (m, 1H), 4.14 (m, 1H), 3.75 (s, 3H), 3.40-3.30 (m, 7H), 3.20-3.10 (m, 3H), 2.88 (m, 2H), 2.81 (s, 3H). MS (ESI) m/z 874.4 (M+H)⁺.

### Example 113

### (7R,16R,21S)-19-chloro-16-[(4,4-difluoropiperidin-1-yl)methyl]-1-(4-fluorophenyl)-10-{[2-(2-methoxyphenyl)pyrimidin-4-yl]methoxy}-20-methyl-7,8,15,16-tetrahydro-18,21-etheno-13,9-(metheno)-6,14,17-trioxa-2-thia-3,5-diazacyclononadeca[1,2,3-cd]indene-7-carboxylic acid

The title compound was prepared as described in Example 82A and Example 82B, substituting 4,4-difluoropiperidine for 1-[2-(methylsulfonyl)ethyl]piperazine. ¹H NMR (501 MHz, dimethyl sulfoxide-d₆) δ ppm 8.89 (d, J=5.1 Hz, 1H), 8.76 (s, 1H), 7.56-7.50 (m, 2H), 7.47 (ddd, J=9.0, 7.4, 1.8 Hz, 1H), 7.25-7.13 (m, 6H), 7.06 (td, J=7.4, 1.0 Hz, 1H), 6.98 (d, J=8.4 Hz, 1H), 6.94 (d, J=9.0 Hz, 1H), 6.87 (dd, J=9.0, 3.0 Hz, 1H), 6.18 (dd, J=5.1, 3.2 Hz, 1H), 5.74 (d, J=2.8 Hz, 1H), 5.25-5.10 (m, 2H), 5.00 (s, 1H), 4.46-4.30 (m, 2H), 3.85 (dd, J=17.1, 5.3 Hz, 1H), 3.77 (s, 3H), 3.16-3.70 (m, 4H), 2.98 (d, J=16.0 Hz, 1H), 2.46-2.26 (m, 6H), 2.24 (s, 3H). MS (ESI) m/z 924.3 (M+H)⁺.

### Example 114

### (7R,16R,21S)-19-chloro-1-(4-fluorophenyl)-10-{[2-(2-methoxyphenyl)pyrimidin-4-yl]methoxy}-20-methyl-16-({methyl[2-(morpholin-4-yl)ethyl]amino}methyl)-7,8,15,16-tetrahydro-18,21-etheno-13,9-(metheno)-6,14,17-trioxa-2-thia-3,5-diazacyclononadeca[1,2,3-cd]indene-7-carboxylic acid

The title compound was prepared as described in Example 82A and Example 82B substituting N-methyl-2-morpholinoethanamine for 1-[2-(methylsulfonyl)ethyl]piperazine. ¹H NMR (400 MHz, dimethyl sulfoxide-d₆) δ ppm 8.89 (d, J=5.1 Hz, 1H), 8.75 (s, 1H), 7.57-7.51 (m, 2H), 7.47 (td, J=7.9, 1.8 Hz, 1H), 7.23-7.10 (m, 6H), 7.06 (t, J=7.5 Hz, 1H), 6.98 (d, J=8.4 Hz, 1H), 6.92 (d, J=9.0 Hz, 1H), 6.86 (dd, J=9.0, 2.9 Hz, 1H), 6.16 (dd, J=5.2, 3.2 Hz, 1H), 5.72 (d, J=2.8 Hz, 1H), 5.17 (q, J=15.0 Hz, 2H), 4.91 (d, J=7.0 Hz, 1H), 4.48-4.24 (m, 3H), 3.93-3.81 (m, 1H), 3.76 (s, 3H), 3.30-2.90 (m, 14H) 2.69 (s, 3H), 2.22 (s, 3H). MS (ESI) m/z 947.0 (M+H)⁺.

### Example 115

### (7R,16R,21S)-19-chloro-1-(4-fluorophenyl)-10-{[2-(2-methoxyphenyl)pyrimidin-4-yl]methoxy}-20-methyl-16-{[(3R,5S)-3,4,5-trimethylpiperazin-1-yl]methyl}-7,8,15,16-tetrahydro-18,21-etheno-13,9-(metheno)-6,14,17-trioxa-2-thia-3,5-diazacyclononadeca[1,2,3-cd]indene-7-carboxylic acid

The title compound was prepared as described in Example 82A and Example 82B substituting (2R,6S)-1,2,6-trimethylpiperazine for 1-[2-(methylsulfonyl)ethyl]piperazine. ¹H NMR (501 MHz, dimethyl sulfoxide-d₆) δ ppm 8.89 (d, J=5.1 Hz, 1H), 8.75 (s, 1H), 7.56-7.50 (m, 2H), 7.50-7.43 (m, 1H), 7.24-7.13 (m, 6H), 7.06 (td, J=7.5, 1.0 Hz, 1H), 6.97 (d, J=8.3 Hz, 1H), 6.91 (d, J=9.0 Hz, 1H), 6.83 (dd, J=9.0, 3.0 Hz, 1H), 6.15 (dd, J=5.3, 3.0 Hz, 1H), 5.67 (d, J=2.8 Hz, 1H), 5.26-5.08 (m, 2H), 4.58 (q, J=6.5 Hz, 1H), 4.47 (d, J=12.9 Hz, 1H), 4.37 (dd, J=13.2, 8.5 Hz, 1H), 3.87 (dd, J=16.9, 5.4 Hz, 1H), 3.77 (s, 3H), 3.72-3.26 (m, 4H), 3.16 (d, J=12.7 Hz, 1H), 2.95-2.85 (m, 2H), 2.82 (s, 3H), 2.76-2.66 (m, 2H), 2.23 (s, 3H), 1.27 (d, J=6.3 Hz, 3H), 1.21 (d, J=6.4 Hz, 3H). MS (ESI) m/z 931.2 (M+H)⁺.

### Example 116

### (7R,16R)-19,23-dichloro-1-(4-fluorophenyl)-10-{[2-(2-methoxyphenyl)pyrimidin-4-yl]methoxy}-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid

### Example 116A

### thieno[2,3-d]pyrimidin-4(3H)-one

A mixture of 2-amino-3-cyanothiophene (50 g) in formic acid (100 mL) and H₂SO₄ (22 mL) was heated in a sealed tube for 2 hours at 100° C. The mixture was cooled to 20° C. and diluted with water (1 L). The resulting precipitate was collected by filtration, washed with water twice (2×1 L) and dried under reduced pressure to provide the title compound. ¹H NMR (400 MHz, dimethyl sulfoxide-d₆) δ ppm 12.16 (br. s., 1H), 8.09 (s, 1H), 7.54 (d, 1H), 7.35 (d, 1H).

### Example 116B

### 5,6-diiodothieno[2,3-d]pyrimidin-4(3H)-one

To an ice-cooled 4-neck 2 L flask fit with a mechanical stirrer, reflux condenser and thermocouple/JKEM was added acetic acid (312 mL), sulfuric acid (9.37 mL) and water (63 mL) with stirring. Example 116A (50 g), periodic acid (37.4 g) and iodine (75 g) were added sequentially and the mixture became slightly endothermic. The ice bucket was removed and a heating mantle was added. The reaction mixture was ramped up to 60° C. and was stirred for 1 hour. Midway through, the temperature climbed to 68-69° C. The heating mantle was removed and the temperature was maintained at 70° C. without external heating. The reaction mixture was cooled to room temperature with an ice bath. The resulting suspension was filtered, and washed with 5:1 acetic acid:water (three times) and ether (five times) to provide the title compound.

### Example 116C

### 4-chloro-5,6-diiodothieno[2,3-d]pyrimidine

A 250 mL flask equipped with magnetic stirring, heating mantle, temperature probe and reflux condenser to a nitrogen bubbler was charged with phosphorus oxychloride (57.3 mL) and N,N-dimethylaniline (17.64 mL). To the mixture was added Example 116B (56.22 g) over 5 minutes. The resulting suspension was heated at 105° C. for 30 minutes. After cooling, the resulting material was broken up and transferred to a funnel with heptane. The material was washed with heptane to remove most of the phosphorus oxychloride. The material was slowly scooped into rapidly stirring ice water (600 mL) and stirred for 30 minutes. The material was collected by filtration, washed with water and ether (200 mL), and dried to provide the title compound which was used in the next step without further purification.

### Example 116D

### 4-chloro-5-iodothieno[2,3-d]pyrimidine

A 500 mL 3-neck jacketed flask with magnetic stirring under nitrogen was charged with Example 116C (23 g) and tetrahydrofuran (200 mL). The resulting suspension was cooled to −16° C. using a Huber chiller set to −17° C. To the mixture was added tert-butylmagnesium chloride (40.8 mL, 2 M in ether) dropwise over 40 minutes, keeping the temperature between −15° C. and −16° C. The temperature was slowly raised to 0° C. and was stirred for 30 minutes. The reaction mixture was cooled to −20° C. and quenched by the very slow dropwise addition (initially about 1 drop/minute) of water (23 mL) over 35 minutes, maintaining the temperature at about −20° C., and then slowly warmed to ambient temperature over 1 hour. The stirring was stopped and the supernatant was decanted from the remaining residue. To the residue was added tetrahydrofuran (200 mL). The mixture was stirred briefly, and after standing, the supernatant was decanted from the remaining residue. This was repeated two times. The combined organics were concentrated. The crude material was purified by chromatography on silica gel eluting with isocratic methylene chloride. The title compound was precipitated from a minimum of hot heptanes.

### Example 116E

### 4-chloro-5-(4-methoxy-2,6-dimethylphenyl)thieno[2,3-d]pyrimidine

To a suspension of Example 116D (5 g), (4-methoxy-2,6-dimethylphenyl)boronic acid (6.07 g) and cesium carbonate (10.99 g) in degassed toluene (50.0 mL) and water (12.5 mL) was added bis(di-tert-butyl(4-dimethylaminophenyl)phosphine)dichloropalladium(II) (597 mg). The mixture was heated to 100° C. overnight. After cooling to room temperature, the mixture was diluted with ethyl acetate (200 mL). The organic layer was washed with water and brine, dried over anhydrous sodium sulfate, filtered and concentrated under vacuum. The residue was purified by silica gel chromatography on a CombiFlash® Teledyne Isco system eluting with 0-20% ethyl acetate in heptanes to provide the title compound. ¹H NMR (501 MHz, CDCl₃) δ ppm 8.88 (s, 1H), 7.35 (s, 1H), 6.70 (s, 2H), 3.85 (s, 3H), 1.99 (s, 6H). MS (ESI) m/z 305.1 (M+H)⁺.

### Example 116F

### 4-chloro-6-iodo-5-(4-methoxy-2,6-dimethylphenyl)thieno[2,3-d]pyrimidine

To a mixture of diisopropylamine (4.15 mL) in tetrahydrofuran (50 mL) cooled to −78° C. was added n-butyllithium (9.71 mL, 2.5 M in hexanes) dropwise. The mixture was stirred for 1 minute before Example 116E (3.7 g) was added as a mixture in tetrahydrofuran (50 mL). The resulting mixture was stirred at −78° C. for 15 minutes. Iodine (6.16 g) was added in one portion and the mixture was warmed to room temperature. The reaction mixture was quenched with saturated aqueous ammonium chloride mixture (100 mL) and was extracted with ethyl acetate (50 mL×3). The combined organic layers were washed sequentially with a sodium thiosulfate mixture and brine, dried over anhydrous sodium sulfate, filtered and concentrated onto silica gel. Purification by flash chromatography on a silica gel column eluting with 0-20% ethyl acetate in heptanes provided crude product, which was triturated with heptanes to obtain the title compound. ¹H NMR (501 MHz, CDCl₃) δ ppm 8.82 (s, 1H), 6.72 (s, 2H), 3.87 (s, 3H), 1.94 (s, 6H). MS (ESI) m/z 431.1 (M+H)⁺.

### Example 116G

### 4-chloro-6-(4-fluorophenyl)-5-(4-methoxy-2,6-dimethylphenyl)thieno[2,3-d]pyrimidine

To a mixture of Example 116F (3.3 g), (4-fluorophenyl)boronic acid (2.144 g) di-tert-butyl(2′,4′,6′-triisopropyl-[1,1′-biphenyl]-2-yl)phosphine (0.179 g) and potassium phosphate tribasic (3.25 g) in degassed tetrahydrofuran (60 mL) and water (15 mL) was added tris(dibenzylideneacetone)dipalladium(0) (0.175 g). The mixture was heated to 60° C. overnight. After cooling to room temperature, the mixture was diluted with ethyl acetate (100 mL). The organic layer was washed with brine, dried over anhydrous sodium sulfate, filtered and concentrated under vacuum. The residue was purified by flash chromatography on a silica gel column eluting with 0-20% ethyl acetate in heptanes to give crude product, which was triturated with heptanes to obtain the title compound. ¹H NMR (501 MHz, CDCl₃) δ ppm 8.84 (s, 1H), 7.31-7.23 (m, 2H), 7.02-6.93 (m, 2H), 6.65 (d, 2H), 3.83 (s, 3H), 1.92 (d, 6H). MS (ESI) m/z 399.1 (M+H)⁺.

### Example 116H

### 4-chloro-5-(3,5-dichloro-4-methoxy-2,6-dimethylphenyl)-6-(4-fluorophenyl)thieno[2,3-d]pyrimidine

To a suspension of Example 116G (2.13 g) in acetonitrile (50 mL) was added N-chlorosuccinimide (2.85 g). The mixture was heated to reflux for 1 hour. The mixture was concentrated under vacuum and the residue was redissolved in ethyl acetate (50 mL). The mixture was washed with brine, dried over anhydrous sodium sulfate, filtered and concentrated under vacuum. The residue was purified by silica gel chromatography on a CombiFlash® Teledyne Isco system eluting with 0-10% ethyl acetate in heptanes to provide the title compound. ¹H NMR (400 MHz, CDCl₃) δ ppm 8.89 (s, 1H), 7.28-7.18 (m, 2H), 7.08-6.97 (m, 2H), 3.96 (s, 3H), 2.02 (s, 6H). MS (ESI) m/z 469.1 (M+H)⁺.

### Example 116I

### 2,6-dichloro-4-(4-chloro-6-(4-fluorophenyl)thieno[2,3-d]pyrimidin-5-yl)-3,5-dimethylphenol

To Example 116H (5 g) in 1,2-dichloroethane (200 mL) was added aluminum trichloride (4.28 g), and the mixture was heated to 68° C. for 6 hours and was cooled to room temperature. Saturated aqueous NaHCO₃ (3 mL) was added and the mixture was stirred for 2 minutes. Saturated aqueous NH₄Cl (15 mL) was added. The mixture was diluted with ethyl acetate and the layers were separated. The aqueous layer was extracted once with ethyl acetate. The organic layers were combined and washed with water and brine, dried over Na₂SO₄, filtered, and concentrated to provide the title compound. ¹H NMR (400 MHz, dimethylsulfoxide-d₆) δ ppm 10.10 (br s, 1H), 9.00 (s, 1H), 7.35 (m, 2H), 7.28 (m, 2H), 1.96 (s, 6H). MS (ESI) m/z 452.9 (M−H)⁻.

### Example 116J

### (R)-3-(allyloxy)propane-1,2-diol

To a 250 mL round bottom with (S)-4-((allyloxy)methyl)-2,2-dimethyl-1,3-dioxolane (7.08 g) was added methanol (100 mL) and p-toluenesulfonic acid monohydrate (0.782 g). The mixture was heated to 50° C. for 18 hours, and at 60° C. for 4 hours. The mixture was cooled to room temperature, and potassium carbonate (1.704 g) and 5 g MgSO₄ were added. The material was filtered and washed with ethyl acetate. The mixture was concentrated, and the residue was chromatographed on silica gel using 20-80% ethyl acetate in heptanes as the eluent, to provide the title compound. ¹H NMR (400 MHz, dimethyl sulfoxide-d₆) δ ppm 5.87 (tdd, 1H), 5.25 (dd, 1H), 5.13 (dd, 1H), 4.62 (d, 1H), 4.46 (t, 1H), 3.94 (ddd, 2H), 3.58 (m, 1H), 3.39 (m, 1H), 3.30 (m, 3H).

### Example 116K

### (S)-1-(allyloxy)-3-(bis(4-methoxyphenyl)(phenyl)methoxy)propan-2-ol

To a mixture of Example 116J (2.25 g) and 4,4′-(chloro(phenyl)methylene)bis(methoxybenzene) (DMTrCl) (6.06 g) in dichloromethane (68.1 mL) cooled to 0° C., was added N,N-diisopropylethylamine (3.27 mL). The mixture was allowed to warm to room temperature and was stirred for 30 minutes. The reaction mixture was quenched with saturated aqueous ammonium chloride mixture (50 mL). The organic layer was washed with brine, dried over anhydrous sodium sulfate, filtered and concentrated under vacuum. The residue was purified by silica gel chromatography on a CombiFlash® Teledyne Isco system, eluting with 0-50% ethyl acetate in heptanes to provide the title compound. ¹H NMR (400 MHz, CDCl₃) δ ppm 7.45-7.40 (m, 2H), 7.35-7.24 (m, 6H), 7.24-7.17 (m, 1H), 6.86-6.77 (m, 4H), 5.95-5.79 (m, 1H), 5.24 (dq, 1H), 5.17 (dq, 1H), 4.00 (dt, 2H), 3.98-3.91 (m, 1H), 3.78 (s, 6H), 3.55 (dd, 1H), 3.49 (dd, 1H), 3.24-3.16 (m, 2H), 2.40 (bs, 1H). MS (ESI) m/z 457.1 (M+Na)+.

### Example 116L

### (R)-5-(4-((1-(allyloxy)-3-(bis(4-methoxyphenyl)(phenyl)methoxy)propan-2-yl)oxy)-3,5-dichloro-2,6-dimethylphenyl)-4-chloro-6-(4-fluorophenyl)thieno[2,3-d]pyrimidine

Triphenylphosphine (1.561 g), Example 116I (1.5 g), and Example 116K (1.580 g) were taken up in 18 mL tetrahydrofuran and di-tert-butylazodicarboxylate (1.370 g) was added and the reaction was stirred overnight. The material was filtered off and rinsed with 1:1 ether/ethyl acetate, and the organics were concentrated. The crude material was chromatographed on silica gel using 1-40% ethyl acetate in heptanes as eluent to provide the title compound. MS (ESI) m/z 891.1 (M+Na)⁺.

### Example 116M

### (R)-ethyl 2-((5-(4-(((R)-1-(allyloxy)-3-(bis(4-methoxyphenyl)(phenyl)methoxy)propan-2-yl)oxy)-3,5-dichloro-2,6-dimethylphenyl)-6-(4-fluorophenyl)thieno[2,3-d]pyrimidin-4-yl)oxy)-3-(5-((tert-butyldimethylsilyl)oxy)-2-((2-(2-methoxyphenyl)pyrimidin-4-yl)methoxy)phenyl)propanoate

To a mixture of Example 116L (2.79 g), Example 68B (2.072 g) and cesium carbonate (2.089 g) was added tert-butanol (30 mL). The suspension was heated to 65° C. overnight. After cooling to room temperature, the mixture was diluted with ethyl acetate (50 mL), washed with water (50 mL) and brine, dried over anhydrous sodium sulfate, filtered and concentrated under vacuum. The residue was purified by silica gel chromatography on a CombiFlash® Teledyne Isco system eluting with 0-75% ethyl acetate in heptanes to provide the title compound. ¹H NMR (400 MHz, CDCl₃) δ 8.81 (d, 1H), 8.54 (s, 1H), 7.69 (dd, 1H), 7.50 (d, 1H), 7.50-7.37 (m, 3H), 7.36-7.25 (m, 4H), 7.28-7.10 (m, 5H), 7.12-7.01 (m, 2H), 6.89-6.78 (m, 2H), 6.82-6.71 (m, 4H), 6.72-6.59 (m, 2H), 6.47 (d, 1H), 5.73 (ddt, 1H), 5.62 (t, 1H), 5.15 (s, 2H), 5.14-5.05 (dq, 1H), 5.03 (dq, 1H), 4.62 (p, 1H), 4.13-3.94 (m, 2H), 3.87 (s, 3H), 3.90-3.82 (m, 2H), 3.82-3.77 (dd, 1H), 3.76 (s, 6H), 3.53 (qd, 2H), 2.94 (dd, 1H), 2.65 (dd, 1H), 2.22 (s, 3H), 1.96 (s, 3H), 1.08 (t, 3H), 0.93 (s, 9H), 0.11 (s, 3H), 0.10 (s, 3H). MS (ESI) m/z 1395.3 (M+Na)⁺.

### Example 116N

### (R)-ethyl 2-((5-(4-(((S)-1-(allyloxy)-3-hydroxypropan-2-yl)oxy)-3,5-dichloro-2,6-dimethylphenyl)-6-(4-fluorophenyl)thieno[2,3-d]pyrimidin-4-yl)oxy)-3-(5-((tert-butyldimethylsilyl)oxy)-2-((2-(2-methoxyphenyl)pyrimidin-4-yl)methoxy)phenyl)propanoate

To a mixture of Example 116M (1.51 g) in dichloromethane (5.5 mL) and methanol (5.50 mL) cooled to 0° C. was added formic acid (5.5 mL). The mixture was stirred at 0° C. for 15 minutes. The mixture was diluted with water (5 mL) and solid sodium bicarbonate was added slowly until pH 7-8 was reached. The mixture was extracted with dichloromethane (3×10 mL) and the combined organic layers were washed with brine, dried over anhydrous sodium sulfate, filtered and concentrated under vacuum to give the crude title compound. The crude material was used in the next step without further purification. LC/MS (ESI) m/z 1070.4 (M+H)⁺.

### Example 116O

### (R)-ethyl 2-((5-(4-(((R)-1-(allyloxy)-3-(tosyloxy)propan-2-yl)oxy)-3,5-dichloro-2,6-dimethylphenyl)-6-(4-fluorophenyl)thieno[2,3-d]pyrimidin-4-yl)oxy)-3-(5-((tert-butyldimethylsilyl)oxy)-2-((2-(2-methoxyphenyl)pyrimidin-4-yl)methoxy)phenyl)propanoate

To a mixture of Example 116N (1.177 g) and p-toluenesulfonyl chloride (0.252 g) in dichloromethane (11 mL) was added triethylamine (0.460 mL). The mixture was allowed to stir at room temperature for 2 hours. Additional p-toluenesulfonyl chloride (0.252 g) and triethylamine (0.460 mL) were added and the mixture was stirred overnight. The mixture was diluted with dichloromethane (10 mL), washed with water and brine, dried over anhydrous sodium sulfate, filtered and concentrated under vacuum. The residue was purified by silica gel chromatography on a CombiFlash® Teledyne Isco system eluting with 0-60% ethyl acetate in heptanes to provide the title compound. ¹H NMR (501 MHz, CDCl₃) δ ppm 8.84 (d, 1H), 8.55 (s, 1H), 7.77-7.73 (m, 2H), 7.71 (dd, 1H), 7.51 (d, 1H), 7.47-7.43 (m, 1H), 7.33-7.26 (m, 5H), 7.26-7.21 (m, 2H), 7.11-6.98 (m, 4H), 6.69 (d, 1H), 6.63 (dd, 1H), 6.45 (d, 1H), 5.80-5.63 (m, 2H), 5.22-5.16 (m, 2H), 5.13 (dq, 1H), 5.08 (dq, 1H), 4.61 (p, 1H), 4.41 (dd, 1H), 4.35 (dd, 1H), 4.14-3.99 (m, 2H), 3.88 (s, 3H), 3.87-3.81 (m, 2H), 3.72-3.65 (m, 2H), 2.97 (dd, 1H), 2.64 (dd, 1H), 2.42 (s, 3H), 2.18 (s, 3H), 1.93 (s, 3H), 1.11 (t, 3H), 0.93 (s, 9H), 0.11 (s, 3H), 0.10 (s, 3H). MS (ESI) m/z 1223.2 (M+H)⁺.

### Example 116P

### (R)-ethyl 2-((5-(4-(((R)-1-(allyloxy)-3-(tosyloxy)propan-2-yl)oxy)-3,5-dichloro-2,6-dimethylphenyl)-6-(4-fluorophenyl)thieno[2,3-d]pyrimidin-4-yl)oxy)-3-(5-hydroxy-2-((2-(2-methoxyphenyl)pyrimidin-4-yl)methoxy)phenyl)propanoate

To a mixture of Example 116O (1.26 g) in tetrahydrofuran (10.29 mL) was added tetrabutylammonium fluoride (1.0 M in tetrahydrofuran, 1.029 mL). The mixture was stirred at room temperature for 10 minutes before quenching with saturated ammonium chloride (10 mL). The mixture was extracted with ethyl acetate (10 mL×3), washed with brine, dried over anhydrous sodium sulfate, filtered and concentrated under vacuum to give the crude title compound. The crude material was used in the next step without further purification. LC/MS (ESI) m/z 1112.5 (M+H)⁺.

### Example 116Q

### ethyl (7R,16R)-19,23-dichloro-1-(4-fluorophenyl)-10-{[2-(2-methoxyphenyl)pyrimidin-4-yl]methoxy}-20,22-dimethyl-16-{[(prop-2-en-1-yl)oxy]methyl}-7,8,15,16-tetrahydro-18,21-etheno-9,13-(metheno)-6,14,17-trioxa-2-thia-3,5-diazacyclononadeca[1,2,3-cd]indene-7-carboxylate

To a mixture of Example 116P (1.14 g) in N,N-dimethylformamide (103.00 mL) was added cesium carbonate (1.68 g). The mixture was stirred at room temperature for 90 minutes. The reaction mixture was poured into water (500 mL) and was extracted with ethyl acetate (3×250 mL). The combined organic layers were washed repeatedly with brine, dried over anhydrous sodium sulfate, filtered and concentrated under vacuum. The residue was purified by silica gel chromatography on a CombiFlash® Teledyne Isco system eluting with 0-80% ethyl acetate in heptanes to provide the title compound. ¹H NMR (400 MHz, CDCl₃) δ ppm 8.90 (d, 1H), 8.62 (s, 1H), 7.70 (dd, 1H), 7.59 (d, 1H), 7.45 (ddd, 1H), 7.13-6.99 (m, 4H), 6.97-6.88 (m, 2H), 6.71 (d, 2H), 6.14 (dd, 1H), 6.05-5.86 (m, 2H), 5.34 (dq, 1H), 5.29-5.09 (m, 4H), 4.58 (dd, 1H), 4.35-4.24 (m, 1H), 4.24-3.97 (m, 4H), 3.96-3.77 (m, 2H), 3.88 (s, 3H), 3.51 (dd, 1H), 3.15 (dd, 1H), 2.22 (s, 3H), 1.90 (s, 3H), 1.08 (t, 3H). MS (ESI) m/z 935.3 (M+H)⁺.

### Example 116R

### ethyl (7R,16R)-19,23-dichloro-1-(4-fluorophenyl)-16-(hydroxymethyl)-10-{[2-(2-methoxyphenyl)pyrimidin-4-yl]methoxy}-20,22-dimethyl-7,8,15,16-tetrahydro-18,21-etheno-9,13-(metheno)-6,14,17-trioxa-2-thia-3,5-diazacyclononadeca[1,2,3-cd]indene-7-carboxylate

To a mixture of Example 116Q (757 mg) in degassed tetrahydrofuran (9 mL) and degassed methanol (6 mL) was added tetrakis(triphenylphosphine)palladium(0) (93 mg) followed by 1,3-dimethylbarbituric acid (315 mg). The mixture was stirred at room temperature overnight. To the mixture was added ammonium pyrrolidinedithiocarbamate (200 mg) and the suspension was stirred for 30 minutes. The mixture was diluted with ethyl acetate (50 mL) and was filtered through diatomaceous earth. The filtrate was concentrated under vacuum and the residue was purified by silica gel chromatography on a CombiFlash® Teledyne Isco system eluting with 0-100% ethyl acetate in heptanes to provide the title compound. ¹H NMR (400 MHz, CDCl₃) δ ppm 8.91 (d, 1H), 8.62 (s, 1H), 7.70 (dd, 1H), 7.61 (d, 1H), 7.45 (ddd, 1H), 7.12-6.99 (m, 4H), 6.99-6.90 (m, 2H), 6.71 (d, 2H), 6.06 (dd, 1H), 5.98 (t, 1H), 5.28-5.21 (m, 1H), 5.17 (dd, 2H), 4.59 (dd, 1H), 4.26-4.19 (m, 1H), 4.19-4.01 (m, 3H), 4.00-3.90 (m, 1H), 3.88 (s, 3H), 3.40 (dd, 1H), 3.22 (dd, 1H), 2.35-2.29 (m, 1H), 2.28 (s, 3H), 1.86 (s, 3H), 1.12 (t, 3H). MS (ESI) m/z 897.4 (M+H)⁺.

### Example 116S

### ethyl (7R,16S)-19,23-dichloro-1-(4-fluorophenyl)-10-{[2-(2-methoxyphenyl)pyrimidin-4-yl]methoxy}-20,22-dimethyl-16-{[(4-methylbenzene-1-sulfonyl)oxy]methyl}-7,8,15,16-tetrahydro-18,21-etheno-9,13-(metheno)-6,14,17-trioxa-2-thia-3,5-diazacyclononadeca[1,2,3-cd]indene-7-carboxylate

To a mixture of Example 116R (700 mg) in dichloromethane (8 mL) and cooled to 0° C. was added p-toluenesulfonyl chloride (223 mg) followed by 1,4-diazabicyclo[2.2.2]octane (175 mg). The mixture was stirred at 0° C. for 15 minutes. The reaction mixture was diluted with dichloromethane (20 mL), washed with saturated aqueous ammonium chloride mixture (20 mL) and brine, dried over anhydrous sodium sulfate, filtered and concentrated under vacuum. The residue was purified by silica gel chromatography on a CombiFlash® Teledyne Isco system eluting with 0-100% ethyl acetate in heptanes to provide the title compound. ¹H NMR (400 MHz, CDCl₃) δ ppm 8.90 (d, 1H), 8.61 (s, 1H), 7.87 (d, 2H), 7.70 (dd, 1H), 7.60 (d, 1H), 7.48-7.41 (m, 1H), 7.38 (d, 2H), 7.12-6.97 (m, 5H), 6.94 (t, 2H), 6.75-6.65 (m, 2H), 6.05 (dd, 1H), 5.91 (d, 1H), 5.23-5.12 (m, 3H), 4.55-4.34 (m, 1H), 4.24-3.98 (m, 1H), 3.88 (s, 3H), 3.41 (dd, 1H), 3.18 (dd, 1H), 2.47 (s, 3H), 2.25 (s, 3H), 1.83 (s, 3H), 1.10 (t, 3H). MS (ESI) m/z 1053.3 (M+H)⁺.

### Example 116T

### ethyl (7R,16R)-19,23-dichloro-1-(4-fluorophenyl)-10-{[2-(2-methoxyphenyl)pyrimidin-4-yl]methoxy}-20,22-dimethyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylate

To a mixture of Example 116S (61 mg) in N,N-dimethylformamide (193 μL) was added 1-methylpiperazine (194 μL). The mixture was heated to 40° C. and was stirred for 24 hours. After cooling to room temperature, the reaction mixture was quenched by addition of acetic acid (100 μL) and further diluted with methanol (2 mL). The mixture was purified by reverse-phase HPLC on a Gilson PLC 2020 using a Luna column (250×50 mm, 10 mm) (10-80% over 45 minutes with acetonitrile in water containing 0.1% trifluoroacetic acid) to provide the title compound after lyophilization. ¹H NMR (501 MHz, dimethyl sulfoxide-d₆) δ ppm 8.92 (d, 1H), 8.75 (s, 1H), 7.57-7.51 (m, 2H), 7.50-7.43 (m, 1H), 7.24-7.11 (m, 5H), 7.05 (t, 1H), 6.93 (d, 1H), 6.85 (dd, 1H), 6.28 (dd, 1H), 5.73 (d, 1H), 5.20 (d, 1H), 5.13 (d, 1H), 4.99-4.88 (m, 1H), 4.48 (dd, 1H), 4.39 (d, 1H), 3.99 (dq, 1H), 3.90 (dq, 1H), 3.76 (s, 3H), 3.40 (bs, 4H), 3.23 (bs, 2H), 3.15-2.93 (m, 5H), 2.88 (qd, 2H), 2.80 (s, 3H), 2.01 (s, 3H), 1.97 (s, 3H), 0.90 (t, 3H). MS (ESI) m/z 979.3 (M+H)⁺.

### Example 116U

### (7R,16R)-19,23-dichloro-1-(4-fluorophenyl)-10-{[2-(2-methoxyphenyl)pyrimidin-4-yl]methoxy}-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid

To a mixture of Example 116T (46 mg) in methanol (529 μL) and tetrahydrofuran (529 μL) was added lithium hydroxide (13.68 mg) in water (529 μL). The mixture was stirred at room temperature for 2.5 hours. Additional lithium hydroxide (13.68 mg) was added and the mixture was allowed to stir overnight. The reaction mixture was quenched by additional of acetic acid (90 μL) and was further diluted with methanol (2 mL). The mixture was purified by reverse-phase HPLC on a Gilson PLC 2020 using a Luna column (250×50 mm, 10 mm) (5-85% over 45 minutes with acetonitrile in water containing 0.1% trifluoroacetic acid). Product containing fractions were combined and lyophilized. The crude material was further purified by reverse-phase HPLC on a Gilson PLC 2020 using a Luna column (250×50 mm, 10 mm) (5-75% over 45 minutes with acetonitrile in water containing 10 mM ammonium acetate) to provide the title compound after lyophilization. ¹H NMR (400 MHz, dimethyl sulfoxide-d₆) δ ppm 8.80 (d, 1H), 8.69 (s, 1H), 7.50-7.44 (m, 2H), 7.39 (ddd, 1H), 7.18-7.02 (m, 5H), 6.98 (td, 1H), 6.84 (d, 1H), 6.48 (s, 1H), 6.20 (dd, 1H), 5.73 (d, 1H), 5.14 (d, 1H), 5.07 (d, 1H), 4.81 (p, 1H), 4.39 (d, 2H), 3.69 (s, 3H), 3.61 (d, 1H), 3.57 (d, 1H), 2.94 (d, 1H), 2.90 (d, 1H), 2.70-2.61 (m, 2H), 2.61-2.43 (m, 6H), 2.29 (s, 3H), 1.93 (s, 3H), 1.89 (s, 3H). MS (ESI) m/z 951.1 (M+H)⁺.

### Example 117

### (7S,16R)-19,23-dichloro-1-(4-fluorophenyl)-10-{[2-(2-methoxyphenyl)pyrimidin-4-yl]methoxy}-20,22-dimethyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid

The title compound was isolated as a minor product during the synthesis and isolation of Example 116U. ¹H NMR (500 MHz, dimethyl sulfoxide-d₆) δ ppm 8.86 (d, 1H), 8.77 (s, 1H), 7.58-7.50 (m, 2H), 7.46 (ddd, 1H), 7.24-7.09 (m, 5H), 7.04 (td, 1H), 6.93 (d, 1H), 6.68 (dd, 1H), 6.42 (dd, 1H), 5.92 (d, 1H), 5.24-5.12 (m, 3H), 4.29-4.20 (m, 2H), 3.76 (s, 3H), 3.19 (dd, 2H), 3.15-3.01 (m, 4H), 2.99-2.83 (m, 2H), 2.80 (s, 3H), 2.04 (s, 3H), 1.83 (s, 3H). MS (ESI) m/z 951.1 (M+H)⁺.

### Example 118

### (7R,16R,21S)-19-chloro-1-(4-fluorophenyl)-10-{[2-(2-methoxyphenyl)pyrimidin-4-yl]methoxy}-20-methyl-16-{[4-(2,2,2-trifluoroethyl)piperazin-1-yl]methyl}-7,8,15,16-tetrahydro-18,21-etheno-13,9-(metheno)-6,14,17-trioxa-2-thia-3,5-diazacyclononadeca[1,2,3-cd]indene-7-carboxylic acid

The title compound was prepared as described in Example 82A and Example 82B substituting 1-(2,2,2-trifluoroethyl)piperazine for 1-[2-(methylsulfonyl)ethyl]piperazine. ¹H NMR (400 MHz, dimethyl sulfoxide-d₆) δ ppm 9.77 (s, 1H), 8.90 (d, J=5.1 Hz, 1H), 8.76 (s, 1H), 7.57-7.52 (m, 2H), 7.50-7.44 (m, 1H), 7.25-7.12 (m, 6H), 7.06 (t, J=7.5 Hz, 1H), 7.00-6.91 (m, 2H), 6.86 (dd, J=9.0, 3.0 Hz, 1H), 6.19 (dd, J=5.1, 3.3 Hz, 1H), 5.75 (d, J=2.8 Hz, 1H), 5.26-5.00 (m, 3H), 4.44-4.28 (m, 2H), 3.77 (s, 3H), 3.56-2.71 (m, 14H), 2.23 (s, 3H). MS (ESI) m/z 971.2 (M+H)⁺.

### Example 119

### (7R,16R,21S)-16-{[bis(2-methoxyethyl)amino]methyl}-19-chloro-1-(4-fluorophenyl)-10-{[2-(2-methoxyphenyl)pyrimidin-4-yl]methoxy}-20-methyl-7,8,15,16-tetrahydro-18,21-etheno-13,9-(metheno)-6,14,17-trioxa-2-thia-3,5-diazacyclononadeca[1,2,3-cd]indene-7-carboxylic acid

The title compound was prepared as described in Example 82A and Example 82B, substituting bis(2-methoxyethyl)amine for 1-[2-(methylsulfonyl)ethyl]piperazine. ¹H NMR (400 MHz, dimethyl sulfoxide-d₆) δ ppm 9.62 (s, 1H), 8.89 (d, J=5.1 Hz, 1H), 8.76 (s, 1H), 7.60-7.41 (m, 3H), 7.24-7.11 (m, 6H), 7.06 (td, J=7.5, 1.0 Hz, 1H), 6.99-6.90 (m, 2H), 6.84 (dd, J=9.0, 3.0 Hz, 1H), 6.20 (dd, J=5.1, 3.3 Hz, 1H), 5.77 (d, J=2.8 Hz, 1H), 5.29-5.09 (m, 3H), 4.51-4.29 (m, 2H), 3.83 (dd, J=17.2, 5.3 Hz, 1H), 3.77 (s, 3H), 3.59-3.40 (m, 10H) 3.29 (s, 6H), 3.06-2.96 (m, 1H), 2.22 (s, 3H). MS (ESI) m/z 936.2 (M+H)⁺.

### Example 120

### (7R,16R,21S)-23-chloro-10-{[2-(2-methoxyphenyl)pyrimidin-4-yl]methoxy}-22-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid

### Example 120A

### 6-bromo-4-chlorothieno[2,3-d]pyrimidine

A stirred mixture of Example 116A (60 g) in POCl₃ (491 mL) was heated to reflux for 6 hours. The mixture was concentrated under reduced pressure to give a residue, which was added to saturated aqueous NaHCO₃ (1.5 L) and was extracted with CH₂Cl₂ (3×1.5 L). The combined organic phase was washed with brine (2 L), dried over Na₂SO₄, filtered, and concentrated to provide the title compound. ¹H NMR (400 MHz, CDCl₃) δ ppm 8.82 (s, 1H), 7.49 (s, 1H).

### Example 120B

### 5-bromo-4-chlorothieno[2,3-d]pyrimidine

To a stirred mixture of Example 120A (28 g) in anhydrous tetrahydrofuran (800 mL) was added dropwise a mixture of lithium diisopropylamide (2M in tetrahydrofuran, 76 mL) at −78° C. The mixture was stirred at −78° C. for 1 hour. A mixture of tetrahydrofuran (150 mL) and water (45 mL) was added dropwise slowly. The mixture was allowed to warm up to 0° C. and was poured into water (1.5 L). The mixture was extracted with CH₂Cl₂ (3×1 L). The combined organic phase was washed with brine, dried over Na₂SO₄, filtered, and concentrated under reduced pressure. The residue was purified by column chromatography on silica gel (eluted with petroleum ether:ethyl acetate=100:1 to 20:1) to give a crude product that was triturated with a mixture of petroleum ether:dichloromethane:ethyl acetate=10:1:1 (500 mL) and filtered. The material was dried under reduced pressure to provide the title compound. ¹H NMR (400 MHz, CDCl₃) δ ppm 8.89 (s, 1H), 7.67 (s, 1H).

### Example 120C

### (R)-ethyl 2-((5-bromo-6-cyclopropylthieno[2,3-d]pyrimidin-4-yl)oxy)-3-(5-((tert-butyldimethylsilyl)oxy)-2-((2-(2-methoxyphenyl)pyrimidin-4-yl)methoxy)phenyl)propanoate

The title compound was prepared as described in Example 108B, replacing Example 108A with Example 120B. MS (APCI) m/z 753.1 (M+H)⁺.

### Example 120D

### (2R)-ethyl 2-((5-((1S)-4-(((R)-1-(bis(4-methoxyphenyl)(phenyl)methoxy)-3-(tosyloxy)propan-2-yl)oxy)-3-chloro-2-methylphenyl)-6-cyclopropylthieno[2,3-d]pyrimidin-4-yl)oxy)-3-(5-((tert-butyldimethylsilyl)oxy)-2-((2-(2-methoxyphenyl)pyrimidin-4-yl)methoxy)phenyl)propanoate

The title compound was prepared as described in Example 108C, replacing Example 108B with Example 120C. MS (ESI) m/z 1345.6 (M+H)⁺.

### Example 120E

### (2R)-ethyl 2-((5-((1S)-4-(((R)-1-(bis(4-methoxyphenyl)(phenyl)methoxy)-3-(tosyloxy)propan-2-yl)oxy)-3-chloro-2-methylphenyl)-6-cyclopropylthieno[2,3-d]pyrimidin-4-yl)oxy)-3-(5-hydroxy-2-((2-(2-methoxyphenyl)pyrimidin-4-yl)methoxy)phenyl)propanoate

The title compound was prepared as described in Example 108D, replacing Example 108C with Example 120D. MS (ESI) m/z 1229.6 (M+H)⁺.

### Example 120F

### ethyl (7R,16R,21S)-16-{[bis(4-methoxyphenyl)(phenyl)methoxy]methyl}-19-chloro-10-{[2-(2-methoxyphenyl)pyrimidin-4-yl]methoxy}-20-methyl-7,8,15,16-tetrahydro-18,21-etheno-9,13-(metheno)-6,14,17-trioxa-2-thia-3,5-diazacyclononadeca[1,2,3-cd]indene-7-carboxylate

The title compound was prepared as described in Example 108E, replacing Example 108D with Example 120E.

### Example 120G

### ethyl (7R,16R,21S)-19-chloro-16-(hydroxymethyl)-10-{[2-(2-methoxyphenyl)pyrimidin-4-yl]methoxy}-20-methyl-7,8,15,16-tetrahydro-18,21-etheno-9,13-(metheno)-6,14,17-trioxa-2-thia-3,5-diazacyclononadeca[1,2,3-cd]indene-7-carboxylate

The title compound was prepared as described in Example 108F, replacing Example 108E with Example 120F. MS (ESI) m/z 755.4 (M+H)⁺.

### Example 120H

### ethyl (7R,16R,21S)-19-chloro-10-{[2-(2-methoxyphenyl)pyrimidin-4-yl]methoxy}-20-methyl-16-{[(4-methylbenzene-1-sulfonyl)oxy]methyl}-7,8,15,16-tetrahydro-18,21-etheno-9,13-(metheno)-6,14,17-trioxa-2-thia-3,5-diazacyclononadeca[1,2,3-cd]indene-7-carboxylate

The title compound was prepared as described in Example 108G, replacing Example 108F with Example 120G. MS (ESI) m/z 909.3 (M+H)⁺.

### Example 120I

### ethyl (7R,16R,21S)-19-chloro-10-{[2-(2-methoxyphenyl)pyrimidin-4-yl]methoxy}-20-methyl-16-[(4-methylpiperazin-1-yl)methyl]-7,8,15,16-tetrahydro-18,21-etheno-9,13-(metheno)-6,14,17-trioxa-2-thia-3,5-diazacyclononadeca[1,2,3-cd]indene-7-carboxylate

The title compound was prepared as described in Example 108H, replacing Example 108G with Example 120H.

### Example 120J

### (7R,16R,21S)-23-chloro-10-{[2-(2-methoxyphenyl)pyrimidin-4-yl]methoxy}-22-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid

The title compound was prepared as described in Example 108I, replacing Example 108H with Example 120I. ¹H NMR (501 MHz, dimethyl sulfoxide-d₆) δ ppm 9.41 (s, 1H), 8.81 (d, 1H), 8.59 (s, 1H), 7.63 (s, 1H), 7.59 (d, 1H), 7.50 (dd, 1H), 7.44 (td, 1H), 7.20 (d, 1H), 7.12 (d, 1H), 7.02 (t, 1H), 6.94 (d, 1H), 6.83 (d, 1H), 6.76 (dd, 1H), 6.05 (d, 1H), 5.68 (dd, 1H), 5.27-5.07 (m, 3H), 4.39 (d, 1H), 4.09 (dd, 1H), 3.73 (s, 3H), 3.55-3.42 (m, 1H), 3.30-3.16 (m, 1H), 3.08 (s, 2H), 2.89 (s, 2H), 2.79 (s, 3H), 2.66-2.52 (m, 2H), 2.31 (s, 3H). MS (ESI) m/z 809.4 (M+H)⁺.

### Example 121

### (7R,16R)-2,19,23-trichloro-1-(4-fluorophenyl)-10-{[2-(2-methoxyphenyl)pyrimidin-4-yl]methoxy}-16-[(4-methylpiperazin-1-yl)methyl]-7,8,15,16-tetrahydro-18,21-etheno-9,13-(metheno)-6,14,17-trioxa-2a,5-diazacyclononadeca[1,2,3-cd]indene-7-carboxylic acid

### Example 121A

### (R)-ethyl 2-acetoxy-3-(5-hydroxy-2-((2-(2-methoxyphenyl)pyrimidin-4-yl)methoxy)phenyl)propanoate

To a solution of Example 68A (2 g) in tetrahydrofuran (34.6 mL) at 0° C. was added tetrabutylammonium fluoride (3.5 mL, 1 M in tetrahydrofuran), and the reaction was allowed to stir at room temperature. The reaction mixture was quenched with saturated aqueous ammonium chloride and water, and the aqueous layer was extracted with ethyl acetate three times. The combined organic layers were washed with water and brine, dried over anhydrous sodium sulfate, filtered and concentrated. The crude residue was purified by normal phase MPLC on a Teledyne Isco Combiflash Rf+ (20-85% ethyl acetate in heptanes) to give the title compound. MS (ESI) m/z 467.1 (M+H)⁺.

### Example 121B

### (2R)-ethyl 2-acetoxy-3-(2-((2-(2-methoxyphenyl)pyrimidin-4-yl)methoxy)-5-((tetrahydro-2H-pyran-2-yl)oxy)phenyl)propanoate

To a solution of Example 121A (1.55 g) in 3,4-dihydro-2H-pyran (2.72 mL) was added p-toluenesulfonic acid monohydrate (2.5 mg), and the reaction was allowed to stir at room temperature. After 30 minutes, p-toluenesulfonic acid monohydrate (63 mg) and dichloromethane (3 mL) were added, and the reaction was allowed to stir. After 3.5 hours, p-toluenesulfonic acid monohydrate (31 mg) and 3,4-dihydro-2H-pyran (1 mL) were added and the reaction was stirred overnight. The reaction mixture was poured into saturated aqueous sodium bicarbonate. The aqueous layer was extracted with ethyl acetate three times, and the combined organic layers were dried over anhydrous sodium sulfate, filtered and concentrated. The residue was purified by normal phase MPLC on a Teledyne Isco Combiflash Rf+(15-75% ethyl acetate in heptanes) to give the title compound. MS (ESI) m/z 551.4 (M+H)⁺.

### Example 121C

### (2R)-ethyl 2-hydroxy-3-(2-((2-(2-methoxyphenyl)pyrimidin-4-yl)methoxy)-5-((tetrahydro-2H-pyran-2-yl)oxy)phenyl)propanoate

To a solution of Example 121B (1.64 g) in ethanol (6 mL) at room temperature was added sodium ethoxide (55 μL, 21% by weight in ethanol), and the reaction was allowed to stir. After 90 minutes, a majority of the ethanol was removed by rotary evaporation, and the residue was taken up in ethyl acetate and water. The aqueous layer was extracted with ethyl acetate three times. The combined organic layers were washed with brine, dried over anhydrous sodium sulfate, filtered and concentrated. The residue was purified by normal phase MPLC on a Teledyne Isco Combiflash Rf+ (20-80% ethyl acetate in heptanes) to give the title compound. MS (ESI) m/z 509.2 (M+H)⁺.

### Example 121D

### (2R)-ethyl 2-((6-chloro-7-(4-fluorophenyl)-8-iodopyrrolo[1,2-a]pyrazin-1-yl)oxy)-3-(2-((2-(2-methoxyphenyl)pyrimidin-4-yl)methoxy)-5-((tetrahydro-2H-pyran-2-yl)oxy)phenyl)propanoate

To a solution of Example 121C (988 mg) and Example 69G (797 mg) in t-butanol (38.9 mL) was added cesium carbonate (1.9 g), and the reaction was warmed to 40° C. overnight. The reaction mixture was cooled, and some t-butanol was removed by rotary evaporation. The residue was taken up in ethyl acetate, water and brine. The aqueous layer was extracted with ethyl acetate three times, and the combined organic layers were washed with water and brine, dried over anhydrous sodium sulfate, filtered and concentrated. The residue was purified by normal phase MPLC on a Teledyne Isco Combiflash Rf+(5-75% ethyl acetate in heptanes) to give the title compound. MS (ESI) m/z 879.2 (M+H)⁺.

### Example 121E

### (R)-ethyl 2-((6-chloro-7-(4-fluorophenyl)-8-iodopyrrolo[1,2-a]pyrazin-1-yl)oxy)-3-(5-hydroxy-2-((2-(2-methoxyphenyl)pyrimidin-4-yl)methoxy)phenyl)propanoate

To a suspension of Example 121D (1.3 g) in cyclopentyl methyl ether (5.4 mL) was added 3 M HCl in cyclopentyl methyl ether (5 mL), and the reaction was allowed to stir. After 30 minutes, the cyclopentyl methyl ether was removed by rotary evaporation. Water, saturated aqueous sodium bicarbonate and ethyl acetate were added to the material, and the aqueous layer was extracted with ethyl acetate three times. The combined organic layers were dried over anhydrous sodium sulfate, filtered and concentrated. The crude residue was purified by normal phase MPLC on a Teledyne Isco Combiflash Rf+ (10-80% ethyl acetate in heptanes) to give the title compound. MS (ESI) m/z 794.9 (M+H)⁺.

### Example 121F

### (R)-ethyl 2-((8-(4-(((R)-1-(bis(4-methoxyphenyl)(phenyl)methoxy)-3-(tosyloxy)propan-2-yl)oxy)-3,5-dichlorophenyl)-6-chloro-7-(4-fluorophenyl)pyrrolo[1,2-a]pyrazin-1-yl)oxy)-3-(5-hydroxy-2-((2-(2-methoxyphenyl)pyrimidin-4-yl)methoxy)phenyl)propanoate

A vial containing Example 88B (238 mg), Example 121E (210 mg), cesium carbonate (258 mg) and bis(di-tert-butyl(4-dimethylaminophenyl)phosphine)dichloropalladium(II) (18.7 mg) was evacuated and backfilled with nitrogen several times. To this vial was added degassed tetrahydrofuran (2.1 mL) and water (530 μL), and the reaction was stirred overnight at room temperature. 1-Pyrrolidinecarbodithioic acid ammonium salt (4.3 mg) was added, and the reaction was allowed to stir for 30 minutes. The reaction mixture was diluted with ethyl acetate and filtered over diatomaceous earth. Brine and water were added, and the aqueous layer was extracted with ethyl acetate three times. The combined organic layers were dried over anhydrous sodium sulfate, filtered and concentrated. The crude residue was purified by normal phase MPLC on a Teledyne Isco Combiflash Rf+ (5-80% ethyl acetate in heptanes) to give the title compound. MS (ESI) m/z 1360.7 (M+H)⁺.

### Example 121G

### ethyl (7R,16S)-16-{[bis(4-methoxyphenyl)(phenyl)methoxy]methyl}-2,19,23-trichloro-1-(4-fluorophenyl)-10-{[2-(2-methoxyphenyl)pyrimidin-4-yl]methoxy}-7,8,15,16-tetrahydro-18,21-etheno-13,9-(metheno)-6,14,17-trioxa-2a,5-diazacyclononadeca[1,2,3-cd]indene-7-carboxylate

A mixture of Example 121F (213 mg) and cesium carbonate (255 mg) in N,N-dimethylformamide (15.8 mL) was stirred at room temperature. After 6 hours, the reaction mixture was transferred to a separatory funnel with water and ethyl acetate. The aqueous layer was extracted with ethyl acetate three times. The combined organic layers were washed with water three times and brine, dried over anhydrous sodium sulfate, filtered and concentrated. The crude residue was purified by normal phase MPLC on a Teledyne Isco Combiflash Rf+ (5-75% ethyl acetate in heptanes) to give the title compound. MS (ESI) m/z 1189.5 (M+H)⁺.

### Example 121H

### ethyl (7R,16R)-2,19,23-trichloro-1-(4-fluorophenyl)-16-(hydroxymethyl)-10-{[2-(2-methoxyphenyl)pyrimidin-4-yl]methoxy}-7,8,15,16-tetrahydro-18,21-etheno-13,9-(metheno)-6,14,17-trioxa-2a,5-diazacyclononadeca[1,2,3-cd]indene-7-carboxylate

To a solution of Example 121G (172 mg) in dichloromethane (730 μL) and methanol (730 μL) was added formic acid (722 μL), and the reaction was allowed to stir. After 30 minutes, the reaction was quenched slowly with saturated aqueous sodium bicarbonate with water bath cooling. The aqueous layer was extracted with ethyl acetate three times, and the combined organic extracts were dried over anhydrous sodium sulfate, filtered and concentrated. The residue was purified by normal phase MPLC on a Teledyne Isco Combiflash Rf+ (15-85% ethyl acetate in heptanes) to give the title compound. MS (ESI) m/z 887.3 (M+H)⁺.

### Example 121I

### ethyl (7R,16S)-2,19,23-trichloro-1-(4-fluorophenyl)-10-{[2-(2-methoxyphenyl)pyrimidin-4-yl]methoxy}-16-{[(4-methylbenzene-1-sulfonyl)oxy]methyl}-7,8,15,16-tetrahydro-18,21-etheno-13,9-(metheno)-6,14,17-trioxa-2a,5-diazacyclononadeca[1,2,3-cd]indene-7-carboxylate

To a solution of Example 121H (103 mg) and triethylamine (81 μL) in dichloromethane (1.1 mL) at room temperature was added p-toluenesulfonyl chloride (66.5 mg), and the reaction was allowed to stir. After 4 hours, the reaction mixture was diluted with dichloromethane and quenched with water. The aqueous layer was extracted with dichloromethane three times, and the combined organic layers were dried over anhydrous sodium sulfate, filtered and concentrated. The crude residue was purified by normal phase MPLC on a Teledyne Isco Combiflash Rf+ (5-75% ethyl acetate in heptanes) to give the title compound. MS (ESI) m/z 1039.4 (M+H)⁺.

### Example 121J

### ethyl (7R,16R)-2,19,23-trichloro-1-(4-fluorophenyl)-10-{[2-(2-methoxyphenyl)pyrimidin-4-yl]methoxy}-16-[(4-methylpiperazin-1-yl)methyl]-7,8,15,16-tetrahydro-18,21-etheno-13,9-(metheno)-6,14,17-trioxa-2a,5-diazacyclononadeca[1,2,3-cd]indene-7-carboxylate

A solution of Example 121I (111 mg) and 1-methylpiperazine (363 μL) in dimethyl formamide (360 μL) was warmed at 38° C. overnight. The reaction was cooled and diluted with ethyl acetate and water. The aqueous layer was extracted with ethyl acetate three times. The combined organic layers were washed with water then brine, dried over anhydrous sodium sulfate, filtered and concentrated. The residue was taken up in dimethyl sulfoxide (2.5 mL) and was purified by RP-HPLC on a Gilson PLC 2020 using a Luna column (250×50 mm, 10 mm) (5-80% over 30 minutes with acetonitrile in water containing 0.1% trifluoroacetic acid) to give the title compound after lyophilyzation. MS (ESI) m/z 969.3 (M+H)⁺.

### Example 121K

### (7R,16R)-2,19,23-trichloro-1-(4-fluorophenyl)-10-{[2-(2-methoxyphenyl)pyrimidin-4-yl]methoxy}-16-[(4-methylpiperazin-1-yl)methyl]-7,8,15,16-tetrahydro-18,21-etheno-9,13-(metheno)-6,14,17-trioxa-2a,5-diazacyclononadeca[1,2,3-cd]indene-7-carboxylic acid

To a solution of Example 121J (69 mg) in tetrahydrofuran (800 μL) and methanol (800 μL) at 0° C. was added a solution of lithium hydroxide (34.5 mg) in water (800 μL), and the reaction was allowed to stir at 0° C. overnight. The reaction was warmed to room temperature and stirred for 6 hours, and quenched with trifluoroacetic acid (133 μL). The mixture was diluted with dimethyl sulfoxide (700 μL) and purified by RP-HPLC on a Gilson PLC 2020 using a Luna column (250×50 mm, 10 mm, 5-75% over 30 minutes with acetonitrile in water containing 0.1% trifluoroacetic acid) to give the title compound after lyophilyzation. ¹H NMR (400 MHz, dimethyl sulfoxide-d₆) δ ppm 8.89 (d, 1H), 7.98 (d, 1H), 7.59 (d, 1H), 7.54 (dd, 1H), 7.50 (d, 1H), 7.49-7.42 (m, 1H), 7.37 (d, 1H), 7.30-7.18 (m, 4H), 7.16 (d, 1H), 7.10-7.00 (m, 2H), 6.90 (d, 1H), 6.73 (dd, 1H) 6.30 (dd, 1H), 6.08 (d, 1H), 5.16 (app q, 2H), 5.06-4.93 (m, 1H), 4.37-4.21 (m, 3H), 3.77 (s, 3H), 3.71 (dd, 1H), 3.52-2.97 (m, 7H), 2.95-2.81 (m, 2H), 2.79 (s, 3H), 2.54 (br s, 2H). MS (ESI) m/z 939.4 (M+H)⁺.

### Example 122

### (7R,16R,21S)-19-chloro-10-{[2-(2-cyanophenyl)pyrimidin-4-yl]methoxy}-1-(4-fluorophenyl)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid

### Example 122A

### (R)-ethyl 2-acetoxy-3-(5-((tert-butyldimethylsilyl)oxy)-2-((2-(2-cyanophenyl)pyrimidin-4-yl)methoxy)phenyl)propanoate

A solution of N¹,N¹,N²,N²-tetramethyldiazene-1,2-dicarboxamide (1.881 g) and triphenylphosphine (2.87 g) were stirred together in tetrahydrofuran (27.3 mL) at 0° C. for 20 minutes. The fine suspension was added to a flask containing Example 100E (1.50 g) and Example 16D (2.090 g) cooled in an ice bath under an atmosphere of nitrogen. The reaction mixture was stirred for 1 hour at 0° C. and was allowed to warm to room temperature and stir overnight. The reaction mixture was filtered, washed with tetrahydrofuran (20 mL) and concentrated. The residue was purified on a silica gel column (Teledyne Isco RediSep® Rf gold 220 g, gradient of 5-40% ethyl acetate/heptanes) to give the title compound. ¹H NMR (400 MHz, chloroform-d) δ ppm 8.95 (d, 1H), 8.41 (d, 1H), 7.87 (d, 1H), 7.78-7.70 (m, 2H), 7.59 (td, 1H), 6.80 (d, 1H), 6.76-6.69 (m, 2H), 5.35 (dd, 1H), 5.32-5.20 (m, 2H), 4.23 (qd, 2H), 3.42 (dd, 1H), 3.03 (dd, 1H), 2.08 (d, 3H), 1.27 (td, 3H), 0.99 (d, 9H), 0.15 (s, 6H). MS (ESI) m/z 576.2 (M+H)⁺.

### Example 122B

### (R)-ethyl 3-(5-((tert-butyldimethylsilyl)oxy)-2-((2-(2-cyanophenyl)pyrimidin-4-yl)methoxy)phenyl)-2-hydroxypropanoate

To a solution of Example 122A (2.65 g) in anhydrous ethanol (23.01 mL) was added 21% sodium ethoxide solution in ethanol (0.086 mL). The reaction was stirred four hours at ambient temperature, then additional 21% sodium ethoxide solution in ethanol (0.086 mL) was added and stirring was continued for 30 minutes. Acetic acid (0.040 mL) was added to the reaction mixture and the mixture was stirred for 10 minutes. The reaction mixture was concentrated and the residue was loaded directly onto a silica gel column (Teledyne Isco RediSep® Rf gold 120 g) and was eluted with a gradient of 5-50% ethyl acetate/heptanes) to give the title compound. ¹H NMR (400 MHz, chloroform-d) δ ppm 8.94 (d, 1H), 8.41 (dd, 1H), 7.87 (dd, 1H), 7.74 (td, 1H), 7.67 (d, 1H), 7.60 (td, 1H), 6.82-6.75 (m, 2H), 6.70 (dd, 1H), 5.30-5.20 (m, 2H), 4.54 (ddd, 1H), 4.31-4.16 (m, 2H), 3.28 (dd, 1H), 3.00 (dd, 1H), 2.84 (d, 1H), 1.28 (t, 3H), 0.98 (s, 9H), 0.18 (s, 6H). MS (ESI) m/z 534.3 (M+H)⁺.

### Example 122C

### (R)-ethyl 2-((5-bromo-6-(4-fluorophenyl)thieno[2,3-d]pyrimidin-4-yl)oxy)-3-(5-((tert-butyldimethylsilyl)oxy)-2-((2-(2-cyanophenyl)pyrimidin-4-yl)methoxy)phenyl)propanoate

A solution of Example 122B (1.98 g), Example 1D (1.339 g) and cesium carbonate (3.63 g) was heated in t-butanol (14.84 mL) under an atmosphere of nitrogen for 3 hours. The reaction mixture was diluted with ethyl acetate (100 mL), washed with water (50 mL) and brine (50 mL), dried over magnesium sulfate, filtered, and concentrated. The residue was loaded onto silica (Teledyne Isco RediSep® Rf gold 120 g) and was eluted using a gradient of 5-50% ethyl acetate/heptanes) to give the title compound. ¹H NMR (400 MHz, chloroform-d) δ ppm 8.93 (d, 1H), 8.52 (s, 1H), 8.40 (d, 1H), 7.87 (d, 1H), 7.78-7.70 (m, 2H), 7.67-7.56 (m, 3H), 7.22-7.15 (m, 2H), 6.97 (d, 1H), 6.80 (d, 1H), 6.69 (dd, 1H), 5.89 (dd, 1H), 5.37-5.19 (m, 2H), 4.34-4.18 (m, 2H), 3.65 (dd, 1H), 3.35 (dd, 1H), 1.27 (t, 3H), 0.95 (s, 9H), 0.13 (s, 3H), 0.12 (s, 3H). MS (ESI) m/z 841.9 (M+H)⁺.

### Example 122D

### (R)-ethyl 2-((5-((1S)-4-(((R)-1-(bis(4-methoxyphenyl)(phenyl)methoxy)-3-(tosyloxy)propan-2-yl)oxy)-3-chloro-2-methylphenyl)-6-(4-fluorophenyl)thieno[2,3-d]pyrimidin-4-yl)oxy)-3-(5-((tert-butyldimethylsilyl)oxy)-2-((2-(2-cyanophenyl)pyrimidin-4-yl)methoxy)phenyl)propanoate

To a mixture of Example 73D (1.799 g), Example 122C (1.577 g), cesium carbonate (1.833 g) and bis(di-tert-butyl(4-dimethylaminophenyl)phosphine)dichloropalladium(II) (0.199 g) in tetrahydrofuran (15.00 mL) and water (3.75 mL) was purged with nitrogen and was stirred for 2 days at room temperature. Additional Pd(amphos)₂Cl₂ (0.199 g) was added, and stirring was continued for another 24 hours. Pyrrolidine-1-carbodithioic acid, ammonia salt (0.046 g) was added and the reaction was stirred for 1 hour. The reaction mixture was diluted with ethyl acetate (100 mL) and was filtered through diatomaceous earth. The organic layer was washed with water (50 mL) and brine (50 mL), dried over magnesium sulfate, filtered, and concentrated. The residue was loaded onto a silica gel column (Teledyne Isco RediSep® Rf gold 120 g) and the column was eluted using a gradient of 5-50% ethyl acetate/heptanes to give the title compound.

### Example 122E

### ethyl (7R,16S,21S)-16-{[bis(4-methoxyphenyl)(phenyl)methoxy]methyl}-19-chloro-10-{[2-(2-cyanophenyl)pyrimidin-4-yl]methoxy}-1-(4-fluorophenyl)-20-methyl-7,8,15,16-tetrahydro-18,21-etheno-13,9-(metheno)-6,14,17-trioxa-2-thia-3,5-diazacyclononadeca[1,2,3-cd]indene-7-carboxylate

To a mixture of Example 122D (0.95 g) in tetrahydrofuran (6.63 mL) was added tetrabutylammonium fluoride (1.0 M in tetrahydrofuran, 0.994 mL) and the reaction was stirred at room temperature. After 20 minutes, the reaction mixture was diluted with ethyl acetate (100 mL), washed with water (50 mL) and brine (50 mL), dried over magnesium sulfate, filtered, and concentrated. The residue was dissolved in N,N-dimethylformamide (65 mL) and was treated with cesium carbonate (1.080 g) and stirred overnight. The reaction mixture was diluted with ethyl acetate (100 mL) and was washed with water (50 mL) and brine (50 mL), dried over magnesium sulfate, filtered, and concentrated. The residue was loaded onto silica gel (Teledyne Isco RediSep® Rf gold 80 g) and was eluted using a gradient of 5-75% ethyl acetate/heptanes to give the title compound. MS (ESI) m/z 1168.1 (M+Na)⁺.

### Example 122F

### ethyl (7R,16R,21S)-19-chloro-10-{[2-(2-cyanophenyl)pyrimidin-4-yl]methoxy}-1-(4-fluorophenyl)-16-(hydroxymethyl)-20-methyl-7,8,15,16-tetrahydro-18,21-etheno-9,13-(metheno)-6,14,17-trioxa-2-thia-3,5-diazacyclononadeca[1,2,3-cd]indene-7-carboxylate

Example 122E (441 mg) in dichloromethane (1.9 mL) and methanol (1.9 mL) was treated with formic acid (14.75 μL) and the reaction was stirred at room temperature. After 30 minutes, the reaction was carefully poured into a mixture of saturated aqueous sodium bicarbonate solution, extracted with dichloromethane (2×25 mL), washed with brine (25 mL), dried over magnesium sulfate, filtered, and concentrated. The residue was loaded onto silica gel (Teledyne Isco RediSep® Rf gold 120 g) and was eluted using a gradient of 5-75% ethyl acetate/heptanes to give the title compound. MS (ESI) m/z 844.1 (M+H)⁺.

### Example 122G

### ethyl (7R,16S,21S)-19-chloro-10-{[2-(2-cyanophenyl)pyrimidin-4-yl]methoxy}-1-(4-fluorophenyl)-20-methyl-16-{[(4-methylbenzene-1-sulfonyl)oxy]methyl}-7,8,15,16-tetrahydro-18,21-etheno-13,9-(metheno)-6,14,17-trioxa-2-thia-3,5-diazacyclononadeca[1,2,3-cd]indene-7-carboxylate

To a solution of Example 122F (250 mg) in dichloromethane (2.0 mL) at 0° C. was added p-toluenesulfonyl chloride (85 mg) followed by DABCO (1,4-diazabicyclo[2.2.2]octane, 66.4 mg). The mixture was stirred at 0° C. for 30 minutes. The reaction was directly loaded onto silica gel (Teledyne Isco RediSep® Rf gold 40 g) and was eluted using a gradient of 5-70% ethyl acetate/heptanes to give the title compound. MS (ESI) m/z 988.3 (M+H)⁺.

### Example 122H

### ethyl (7R,16R,21S)-19-chloro-10-{[2-(2-cyanophenyl)pyrimidin-4-yl]methoxy}-1-(4-fluorophenyl)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

To a solution of Example 122G (285 mg) in dimethylformamide (1.0 mL) was added 1-methylpiperazine (950 μL) and the reaction was stirred at 35° C. under nitrogen for 20 hours. The reaction mixture was cooled, diluted with ethyl acetate (50 mL), washed with water (2×25 mL) and brine (25 mL), dried over magnesium sulfate, filtered, and concentrated to give the title compound. MS (ELSD) m/z 926.4 (M+H)⁺.

### Example 122I

### (7R, 16R,21S)-19-chloro-10-{[2-(2-cyanophenyl)pyrimidin-4-yl]methoxy}-1-(4-fluorophenyl)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid

To a solution of Example 122H (0.125 g) in tetrahydrofuran (0.818 mL) and methanol (0.818 mL) was added a solution of lithium hydroxide (0.048 g) in water (1.00 mL). The reaction was stirred overnight. The reaction was quenched with a solution of N,N-dimethylformamide (0.75 mL) and water (0.25 mL) containing 2,2,2-trifluoroacetic acid (0.177 mL). The resulting solution was purified by Prep HPLC using a Gilson 2020 system (Luna column, 250×50 mm, flow 70 mL/minute) using a gradient of 5-75% acetonitrile/water containing trifluoroacetic acid over 45 minutes. The product containing fractions were lyophilized. The material was further purified by Prep HPLC using a Gilson 2020 system (Luna column, 250×50 mm, flow 70 mL/minute) using a gradient of 10-85% acetonitrile/water containing 10 nM ammonium acetate over 45 minutes. Desired product containing fractions were lyophilized to give the title compound. ¹H NMR (501 MHz, dimethyl sulfoxide-d₆) δ ppm 8.99 (d, 1H), 8.71 (s, 1H), 8.32 (dd, 1H), 7.99 (dd, 1H), 7.85 (td, 1H), 7.72 (td, 1H), 7.63 (d, 1H), 7.20-7.13 (m, 3H), 7.10 (d, 1H), 6.92 (d, 1H), 6.87 (d, 1H), 6.74 (dd, 1H), 6.13 (dd, 1H), 5.66 (d, 1H), 5.31-5.18 (m, 2H), 4.51 (q, 1H), 4.45 (d, 1H), 4.28 (dd, 1H), 3.87 (dd, 1H), 2.92-2.83 (m, 2H), 2.60-2.49 (m, 2H), 2.46-2.31 (m, 8H), 2.21 (s, 3H), 2.19 (s, 3H). MS (ESI) m/z 898.4 (M+H)⁺.

### Example 123

### (7R,20R)-18-chloro-10-{[2-(3-fluoro-2-methoxyphenyl)pyrimidin-4-yl]methoxy}-1-(4-fluorophenyl)-19-methyl-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylic acid

### Example 123A

### (2-(3-fluoro-2-methoxyphenyl)pyrimidin-4-yl)methanol

To a solution of (3-fluoro-2-methoxyphenyl)boronic acid (1.71 g) and (2-chloropyrimidin-4-yl)methanol (1.45 g) in tetrahydrofuran (30 mL) was added tetrakis(triphenylphosphine)palladium(0) (580 mg) and saturated aqueous NaHCO₃ (40 mL). The mixture was stirred under nitrogen at 70° C. overnight. The mixture was concentrated under vacuum and the residue was diluted with water (60 mL), and ethyl acetate (300 mL). The organic layer was separated, washed with water and brine, dried over Na₂SO₄, and filtered. Evaporation of the solvent gave crude product which was loaded on an 80 g column (Grace) and was eluted with 20% ethyl acetate in dichloromethane to give the title compound. MS (ESI) m/z 235.1 (M+H)⁺.

### Example 123B

### 4-(chloromethyl)-2-(3-fluoro-2-methoxyphenyl)pyrimidine

To a solution of Example 123A (234 mg) in dioxane (6 mL) was added (chloromethylene)dimethyliminium chloride (160 mg). The mixture was stirred for 45 minutes. The mixture was diluted with ethyl acetate (100 mL), washed with aqueous NaHCO₃, water, and brine, dried over Na₂SO₄, and filtered. Evaporation of the solvent and column (24 g Grace) purification (20% ethyl acetate in heptane) provided the title compound. MS (ESI) m/z 253.1 (M+H)⁺.

### Example 123C

### ethyl (7R,20S)-18-chloro-10-{[2-(3-fluoro-2-methoxyphenyl)pyrimidin-4-yl]methoxy}-1-(4-fluorophenyl)-19-methyl-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylate

The title compound was prepared as described in Example 65N, substituting Example 123B for Example 65E. MS (ESI) m/z 946.4 (M+H)⁺.

### Example 123D

### (7R,20S)-18-chloro-10-{[2-(3-fluoro-2-methoxyphenyl)pyrimidin-4-yl]methoxy}-1-(4-fluorophenyl)-19-methyl-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylic acid

The title compound was prepared as described in Example 10F, substituting Example 123C for Example 10E. ¹H NMR (400 MHz, dimethyl sulfoxide-d₆) δ ppm 8.75 (d, 1H), 8.63 (s, 1H), 7.56-7.39 (m, 3H), 7.36-7.21 (m, 7H), 7.19-7.10 (m, 2H), 6.87 (d, 1H), 6.49 (d, 1H), 5.94 (dd, 1H), 5.31-5.02 (m, 2H), 4.38 (d, 2H), 4.18 (s, 2H), 3.84 (s, 3H), 3.26-3.13 (m, 2H), 3.04 (p, 2H), 2.80 (s, 3H), 1.73 (s, 3H). MS (ESI) m/z 918.5 (M+H)⁺.

### Example 124

### (7R,20S)-18-chloro-10-{[2-(5-fluoro-2-methoxyphenyl)pyrimidin-4-yl]methoxy}-1-(4-fluorophenyl)-19-methyl-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylic acid

### Example 124A

### (2-(5-fluoro-2-methoxyphenyl)pyrimidin-4-yl)methanol

To a solution of (5-fluoro-2-methoxyphenyl)boronic acid (1.71 g) and (2-chloropyrimidin-4-yl)methanol (1.45 g) in tetrahydrofuran (30 mL) was added Pd(Ph₃P)₄ (tetrakis(triphenylphosphine)palladium(0), 580 mg) and saturated aqueous NaHCO₃ (40 mL). The mixture was stirred under nitrogen at 70° C. overnight. The mixture was concentrated under vacuum and the residue was diluted with water (60 mL) and ethyl acetate (300 mL). The organic layer was separated, washed with water and brine, dried over Na₂SO₄, and filtered. Evaporation of solvent gave crude product which was loaded on an 80 g column (Grace) and was eluted with 20% ethyl acetate in dichloromethane to give the title compound. MS (ESI) m/z 235.1 (M+H)⁺.

### Example 124B

### 4-(chloromethyl)-2-(5-fluoro-2-methoxyphenyl)pyrimidine

To a solution of Example 124A (234 mg) in dioxane (6 mL) was added (chloromethylene)dimethyliminium chloride (160 mg). The mixture was stirred at room temperature for 45 minutes. LC/MS showed the desired product as a major peak. The mixture was diluted with ethyl acetate (100 mL), washed with aqueous NaHCO₃, water, and brine, dried over Na₂SO₄, and filtered. Evaporation of solvent and column (24 g Grace) purification (20% ethyl acetate in heptane) provided the title compound. MS (ESI) m/z 253.1 (M+H)⁺.

### Example 124C

### ethyl (7R,20S)-18-chloro-10-{[2-(5-fluoro-2-methoxyphenyl)pyrimidin-4-yl]methoxy}-1-(4-fluorophenyl)-19-methyl-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylate

The title compound was prepared as described in Example 65N, substituting Example 124B for Example 65E. MS (ESI) m/z 946.4 (M+H)⁺.

### Example 124D

### (7R,20S)-18-chloro-10-{[2-(5-fluoro-2-methoxyphenyl)pyrimidin-4-yl]methoxy}-1-(4-fluorophenyl)-19-methyl-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylic acid

The title compound was prepared as described in Example 10F substituting Example 124C for Example 10E. ¹H NMR (501 MHz, dimethyl sulfoxide-d₆) δ ppm 8.70 (d, 1H), 8.64 (s, 1H), 7.54 (d, 1H), 7.39-7.30 (m, 3H), 7.27-7.22 (m, 4H), 7.21-7.13 (m, 3H), 6.89 (d, 1H), 6.50 (d, 1H), 5.95 (dd, 1H), 5.25-4.98 (m, 2H), 4.58-4.34 (m, 2H), 4.24 (q, 2H), 3.76 (s, 3H), 3.58 (q, 3H), 3.31-2.98 (m, 4H), 2.82 (s, 3H), 1.75 (s, 3H). MS (ESI) m/z 918.3 (M+H)⁺.

### Example 125

### (7R,20S)-18-chloro-1-(4-fluorophenyl)-10-{[2-(4-hydroxyphenyl)pyrimidin-4-yl]methoxy}-19-methyl-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylic acid

### Example 125A

### methyl 2-(4-((tert-butyldimethylsilyl)oxy)phenyl)pyrimidine-4-carboxylate

A mixture of methyl 2-chloropyrimidine-4-carboxylate (3.57 g) and 4-(tert-butyldimethylsilyloxy)phenylboronic acid (15.7 g) were suspended in previously degassed 1,4-dioxane, (140 mL). Potassium carbonate (10.75 g) was solubilized in previously degassed water (21.5 mL), and was added to the reaction mixture. 1,1′-Bis(diphenylphosphino)ferrocene-palladium(II)dichloride dichloromethane complex (2.050 g) was then added and the reaction mixture was placed under an argon atmosphere, then heated at 80° C. for 7 hours. The reaction mixture was diluted with 250 mL of dichloromethane and 200 mL of water and the layers were separated. The aqueous layer was extracted with 3×150 mL of dichloromethane. The combined organic layers were dried over MgSO₄, filtered, and concentrated to provide the crude material. Purification was performed by flash chromatography on a Biotage® silica gel cartridge (KPSil 340 g), eluting from 5-20% ethyl acetate in cyclohexane to afford the title compound. LC/MS (APCI) m/z 345.0 (M+H)⁺.

### Example 125B

### (2-(4-((tert-butyldimethylsilyl)oxy)phenyl)pyrimidin-4-yl)methanol

To a solution of Example 125A (14.06 g) in tetrahydrofuran (100 mL) and methanol (200 mL) was added at −10° C., sodium borohydride (5.40 g) and the reaction was stirred at 0° C. for 30 minutes. The reaction was quenched at 0° C. with 400 mL saturated aqueous NH₄Cl and the organic solvents were evaporated. The remaining mixture was diluted with 300 mL dichloromethane. The organic layer was collected and the aqueous phase was extracted with 3×200 mL dichloromethane. The organic layers were combined, dried with MgSO₄, filtered and concentrated. The crude material was purified on a silica gel column eluting with 5-20% ethyl acetate in cyclohexane to afford the title compound. LC/MS (APCI) m/z 317.0 (M+H)⁺.

### Example 125C

### 4-(4-(hydroxymethyl)pyrimidin-2-yl)phenol

To an ambient solution of Example 125B (1.5 g) in tetrahydrofuran (60 mL) was added tetrabutylammonium fluoride (5.21 mL, 1.0 M in tetrahydrofuran) via syringe. The reaction was stirred overnight and was quenched by the addition of methanol (30 mL). The mixture was concentrated under reduced pressure. The residue was purified by silica gel chromatography (50 g), eluting with a gradient of 0-5% methanol in dichloromethane to give the title compound. ¹H NMR (300 MHz, dimethyl sulfoxide-d₆) δ ppm 9.92 (s, 1H), 8.78 (d, 1H), 8.23 (d, 2H), 7.37 (d, 1H), 6.86 (d, 2H), 5.62 (t, 1H), 4.59 (d, 2H).

### Example 125D

### (2-(4-((2-(trimethylsilyl)ethoxy)methoxy)phenyl)pyrimidin-4-yl)methanol

To a cold (0° C.) solution of Example 125C (30 mg) in tetrahydrofuran (1 mL) was added sodium hydride (6 mg, 60% in mineral oil) followed by 2-(trimethylsilyl)ethoxymethyl chloride (25 mg). The cold bath was removed, and the reaction was stirred for 24 hours. The reaction mixture was quenched by the slow addition of methanol (0.5 mL) and saturated aqueous sodium bicarbonate solution (5 mL). The layers were separated, and the aqueous layer was extracted with additional dichloromethane (3×10 mL). The combined organic layers were dried with magnesium sulfate, filtered and concentrated under reduced pressure. The residue was purified by silica gel chromatography (10 g), eluting with a gradient of 10-25% ethyl acetate in cyclohexane to give the title compound. MS (ESI) m/z 332.9 (M+H)⁺.

### Example 125E

### 4-(chloromethyl)-2-(4-((2-(trimethylsilyl)ethoxy)methoxy)phenyl)pyrimidine

To a cold (0° C.) solution of Example 125D (296 mg) in dichloromethane was added triphenylphosphine (420 mg) followed by 1-chloropyrrolidine-2,5-dione (178 mg). The reaction was stirred at 0° C. for 5 hours. The reaction mixture was loaded directly to a silica gel column (20 g) and was eluted with a gradient of 10-50% ethyl acetate in cyclohexane to give the title compound. MS (ESI) m/z 351.2 (M+H)⁺.

### Example 125F

### ethyl (7R,20S)-18-chloro-1-(4-fluorophenyl)-19-methyl-15-[2-(4-methylpiperazin-1-yl)ethyl]-10-{[2-(4-{[2-(trimethylsilyl)ethoxy]methoxy}phenyl)pyrimidin-4-yl]methoxy}-7,8,15,16-tetrahydro-14H-17,20-etheno-13,9-(metheno)-6-oxa-2-thia-3,5,15-triazacyclooctadeca[1,2,3-cd]indene-7-carboxylate

To a mixture of Example 125E (144 mg) and Example 65M (300 mg) in N,N-dimethylformamide (1.2 mL) was added cesium carbonate (402 mg), and the reaction mixture was stirred for 2.5 hours. The reaction was diluted with water, and the sample was purified directly by reverse-phase HPLC (Kinetex XB C-18 30×150 mm column, 42 mL/minute flow rate), eluting with a gradient of 10-100% acetonitrile in water containing 0.1 v/v formic acid. The fractions containing the desired product were lyophilized to give the title compound. MS (ESI) m/z 1044.5 (M+H)⁺.

### Example 125G

### ethyl (7R,20S)-18-chloro-1-(4-fluorophenyl)-10-{[2-(4-hydroxyphenyl)pyrimidin-4-yl]methoxy}-19-methyl-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylate

To a cold (0° C.) mixture of Example 125F (108 mg) in tetrahydrofuran (3.0 mL) and methanol (3.0 mL) was added concentrated sulfuric acid (6 μL). The ice bath was removed, and the reaction was stirred for an additional 5 hours. Saturated aqueous sodium bicarbonate solution (15 mL) was cautiously added to the solution, and the mixture was extracted with dichloromethane (3×30 mL). The combined organic layers were dried with anhydrous magnesium sulfate, filtered and concentrated under reduced pressure to give the title compound, which was used in the next step without further purification. MS (ESI) m/z 914.4 (M+H)⁺.

### Example 125H

### (7R,20S)-18-chloro-1-(4-fluorophenyl)-10-{[2-(4-hydroxyphenyl)pyrimidin-4-yl]methoxy}-19-methyl-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylic acid

To Example 125G (93 mg) in a mixture of 1,4-dioxane (2.5 mL) and water (2.5 mL) was added lithium hydroxide hydrate (42.7 mg). The resulting mixture was stirred at room temperature for 15 hours and was quenched by the addition of water and 1N aqueous HCl solution until neutral. The mixture was extracted twice with chloroform. The combined organic layers were dried with anhydrous magnesium sulfate, filtered and concentrated under reduced pressure. The residue was dissolved in tetrahydrofuran and was passed through a 0.45 m filter. The eluent was lyophilized to provide the title compound. ¹H NMR (500 MHz, dimethyl sulfoxide-d₆) δ ppm (10.20 (br s, 1H), 8.54 (s, 1H), 8.47 (d, 1H), 8.23 (s, 1H), 8.18 (d, 2H), 7.39 (d, 1H), 7.24 (d, 1H), 7.18 (dd, 2H), 7.11 (dd, 2H), 7.06 (d, 1H), 6.92 (d, 1H), 6.86 (d, 1H), 6.64 (m, 2H), 5.85 (d, 1H), 5.08 (d, 1H), 4.95 (d, 1H), 3.82 (d, 2H), 3.66 (m, 2H), 3.50 (d, 2H), 3.24 (d, 2H), 3.01 (m, 2H), 2.88 (m, 4H), 2.60 (m, 4H), 2.19 (s, 3H), 2.18 (s, 3H). MS (ESI) m/z 886.3 (M+H)⁺.

### Example 126

### (7R,16R)-1-(4-fluorophenyl)-10-{[2-(2-methoxyphenyl)pyrimidin-4-yl]methoxy}-16-[(4-methylpiperazin-1-yl)methyl]-7,8,15,16-tetrahydro-18,21-etheno-9,13-(metheno)-2,6,14,17-tetraoxa-3,5-diazacyclononadeca[1,2,3-cd]indene-7-carboxylic acid

### Example 126A

### (S)-3-(bis(4-methoxyphenyl)(phenyl)methoxy)-2-(4-bromophenoxy)propyl 4-methylbenzenesulfonate

Example 112B (200 mg), 4-bromophenol (76 mg) and triphenylphosphine (143 mg) were mixed under an argon atmosphere. Tetrahydrofuran (3.6 mL) was added followed by addition of trimethylamine (76 μL). Subsequently di-tert-butyl azodicarboxylate (126 mg) was dissolved in tetrahydrofuran (1.6 mL) and was added to the reaction mixture. After stirring for 3 days at room temperature, ethyl acetate and water were added. The aqueous phase was extracted with ethyl acetate. The combined organic extracts were dried over MgSO₄, and filtered. The solvent was reduced in vacuo. The residue was purified by a short silica gel flash chromatography (10% ethyl acetate in heptane) to give the title compound which was directly used in the next step.

### Example 126B

### (R)-1-(3-(bis(4-methoxyphenyl)(phenyl)methoxy)-2-(4-bromophenoxy)propyl)-4-methylpiperazine

A solution of Example 126A (300 mg), 1-methylpiperazine (96 mg) and triethylamine (80 μL) in N,N-dimethylformamide (2 mL) was heated to 140° C. for 1 hour. Ethyl acetate was added and the organic phase was washed twice with water and brine. The organic layer was dried over MgSO₄, filtered, and concentrated in vacuo. The residue obtained was purified by silica gel flash chromatography (12 g Chromabond® column, gradient methanol in dichloromethane 0-4.8%) to give the title compound. MS (ESI) m/z 329.25/331.30 ([M−DMTr]+H)⁺.

### Example 126C

### (R)-1-(3-(bis(4-methoxyphenyl)(phenyl)methoxy)-2-(4-(4,4,5,5-tetramethyl-1,3,2-dioxaborolan-2-yl)phenoxy)propyl)-4-methylpiperazine

A solution of Example 126B (75 mg) in 2-methyltetrahydrofuran (1.5 mL) was degassed and added to a mixture of potassium acetate (23.3 mg), 1,1′-bis(diphenylphosphino)ferrocene-palladium (II) dichloride dichloromethane complex (4.9 mg) and bis(pinacolato)diboron (36.2 mg). The reaction mixture was heated for 16 hours at 90° C. Additional 1,1′-bis(diphenylphosphino)ferrocene-palladium (II) dichloride dichloromethane complex (4.9 mg) was added and the reaction mixture was heated for an additional 16 hours at 90° C. Ethyl acetate was added to the reaction mixture and the mixture was filtered through diatomaceous earth. The solvent was removed in vacuo and the crude product was purified by silica gel flash chromatography (4 g Chromabond® column, gradient ethanol in ethyl acetate 0-60%) to give the title compound. MS (ESI) m/z 377.40 ([M−DMTr]+H)⁺.

### Example 126D

### (R)-ethyl 2-((5-(4-(((S)-1-(bis(4-methoxyphenyl)(phenyl)methoxy)-3-(4-methylpiperazin-1-yl)propan-2-yl)oxy)phenyl)-6-(4-fluorophenyl)furo[2,3-d]pyrimidin-4-yl)oxy)-3-(5-((tert-butyldimethylsilyl)oxy)-2-((2-(2-methoxyphenyl)pyrimidin-4-yl)methoxy)phenyl)propanoate

A mixture of Example 68C (40 mg), Example 126C (40.9 mg), cesium carbonate (47.1 mg) and bis (di-tert-butyl(4-dimethylaminophenyl)phosphine)dichloropalladium (II) (3.4 mg) were stirred under argon. A solution of tetrahydrofuran (1.2 mL) and water (0.3 mL) was degassed and was added. After stirring for 48 hours at room temperature, water was added and the mixture was extracted with ethyl acetate. The combined organic layers were washed with water, dried over MgSO₄, filtered, and concentrated in vacuo. The residue obtained was used without any further purification in the next step. MS (ESI) m/z 999.55 ([M−DMTr]+H)⁺.

### Example 126E

### (R)-ethyl 3-(5-((tert-butyldimethylsilyl)oxy)-2-((2-(2-methoxyphenyl)pyrimidin-4-yl)methoxy)phenyl)-2-((6-(4-fluorophenyl)-5-(4-(((S)-1-hydroxy-3-(4-methylpiperazin-1-yl)propan-2-yl)oxy)phenyl)furo[2,3-d]pyrimidin-4-yl)oxy)propanoate

Formic acid (136 mg) was added to a solution of Example 126D (77 mg) in dichloromethane/methanol (0.4 mL/0.4 mL) and the reaction mixture was stirred for 48 hours at room temperature. The pH was adjusted to 9 under ice-cooling using saturated aqueous NaHCO₃ solution. After extraction with ethyl acetate, the combined organic layers were washed with water, dried over MgSO₄, filtered, and concentrated in vacuo. The residue obtained was purified by silica gel flash chromatography (4 g Chromabond® column, gradient methanol in dichloromethane 1-10%) to give the title compound. MS (ESI) m/z 999.50 (M+H)⁺.

### Example 126F

### (R)-ethyl 2-((6-(4-fluorophenyl)-5-4-(((S)-1-hydroxy-3-(4-methylpiperazin-1-yl)propan-2-yl)oxy)phenyl)furo[2,3-d]pyrimidin-4-yl)oxy)-3-(5-hydroxy-2-((2-(2-methoxyphenyl)pyrimidin-4-yl)methoxy)phenyl)propanoate

TBAF (tetrabutyl ammonium fluoride, 135 μL, 1M solution in tetrahydrofuran) was added to a solution of Example 126E (90 mg) in tetrahydrofuran (2 mL). After stirring for 15 minutes at room temperature, aqueous ammonium chloride solution (10%) was added and the mixture was extracted with ethyl acetate. The combined extracts were washed with water, dried over MgSO₄, filtered, and the solvent was reduced in vacuo. The residue obtained was purified by silica gel flash chromatography (4 g Chromabond® column, gradient methanol in dichloromethane 1-15%) to give the title compound. MS (ESI) m/z 885.40 (M+H)⁺.

### Example 126G

### ethyl (7R,16R)-1-(4-fluorophenyl)-10-{[2-(2-methoxyphenyl)pyrimidin-4-yl]methoxy}-16-[(4-methylpiperazin-1-yl)methyl]-7,8,15,16-tetrahydro-18,21-etheno-9,13-(metheno)-2,6,14,17-tetraoxa-3,5-diazacyclononadeca[1,2,3-cd]indene-7-carboxylate

Example 126F (45.0 mg) and triphenylphosphine (40.0 mg) were mixed in a vial under argon. Tetrahydrofuran (2 mL) was added. Subsequently, di-tert-butyl azodicarboxylate (35.0 mg) was added. After stirring for 64 hours at room temperature, water was added and the mixture was extracted with ethyl acetate. The combined extracts were dried over MgSO₄, filtered, and the solvent was reduced in vacuo. The residue was purified by preparative HPLC (Waters X-Bridge C18 19×150 mm 5 μm column, gradient 5-100% acetonitrile+0.1% trifluoroacetic acid in water+0.1% trifluoroacetic acid) to give the title compound. MS (ESI) m/z 867.40 (M+H)⁺.

### Example 126H

### (7R,16R)-1-(4-fluorophenyl)-10-{[2-(2-methoxyphenyl)pyrimidin-4-yl]methoxy}-16-[(4-methylpiperazin-1-yl)methyl]-7,8,15,16-tetrahydro-18,21-etheno-9,13-(metheno)-2,6,14,17-tetraoxa-3,5-diazacyclononadeca[1,2,3-cd]indene-7-carboxylic acid

LiOH (18.8 mg) was added to a solution of Example 126G (27 mg) in tetrahydrofuran/water (1.0 mL/0.4 mL). The reaction mixture was stirred for 3 days at room temperature. 2,2,2-Trifluoroacetic acid (65 μL) was added to the reaction mixture. The solvent was removed in vacuo. Purification by HPLC (Waters X-Bridge C18 19×150 mm 5 m column, gradient 5-100% acetonitrile+0.1% trifluoroacetic acid in water+0.1% trifluoroacetic acid) provided the title compound ¹H NMR (400 MHz, methanol-d) δ ppm 8.82 (d, 1H), 8.42 (s, 1H), 7.76 (d, 1H), 7.64-7.58 (m, 5H), 7.49 (m, 1H), 7.13-7.05 (m, 6H), 6.79 (m, 1H), 6.74 (m, 1H), 6.37 (d, 1H), 5.90 (dd, 1H), 5.18 (m, 2H), 5.03 (m, 1H), 4.35 (m, 1H), 4.14 (m, 1H), 3.84 (s, 3H), 3.45-3.30 (m, 5H), 3.25-3.15 (m, 5H), 2.90 (m, 5H). MS (ESI) m/z 839.4 (M+H)⁺.

### Example 127

### (7R,16R)-19,23-dichloro-1-(4-fluorophenyl)-10-{[2-(2-methoxyphenyl)pyrimidin-4-yl]methoxy}-16-[(4-methylpiperazin-1-yl)methyl]-7,8,15,16-tetrahydro-14H-18,21-etheno-9,13-(metheno)-6,17-dioxa-2-thia-3,5-diazacyclononadeca[1,2,3-cd]indene-7-carboxylic acid

### Example 127A

### (S)-2,2-dimethyl-4-vinyl-1,3-dioxolane

To a solution of (S)-but-3-ene-1,2-diol (8.8 g) and 2,2-dimethoxypropane (20.8 g) in dichloromethane (60 mL) was added para-toluenesulfonic acid monohydrate (0.42 g). The reaction mixture was stirred at room temperature overnight. The mixture was diluted with ether, and washed with water/brine. The organic layer was dried over Na₂SO₄, filtered, and concentrated carefully under vacuum to give the title compound. ¹H NMR (400 MHz, CDCl₃) δ ppm 5.86 (m, 1H), 5.37 (d, 1H), 5.32 (d, 1H), 4.49 (dd, 1H), 4.10 (dd, 1H), 3.60 (t, 1H), 1.43 (s, 3H), 1.40 (s, 3H).

### Example 127B

### (2R)-ethyl 2-acetoxy-3-(5-((E)-2-(2,2-dimethyl-1,3-dioxolan-4-yl)vinyl)-2-((2-(2-methoxyphenyl)pyrimidin-4-yl)methoxy)phenyl)propanoate

To a 100 mL round bottom flask was added Example 1L (3.3 g), Example 127A (1.5 g), tri-O-tolylphosphine (379 mg), palladium(II) acetate (140 mg), and N,N-diisopropylethylamine (40 mL). The reaction mixture was purged with argon and was stirred at 95° C. overnight. The reaction mixture was cooled to room temperature, diluted with ethyl acetate (300 mL), washed with water and brine, dried over Na₂SO₄, and filtered. Evaporation of the solvent and column purification (20% ethyl acetate in dichloromethane) of the crude material provided the title compound. MS (ESI) m/z 577.3 (M+H)⁺.

### Example 127C

### (2R)-ethyl 2-acetoxy-3-(5-(2-(2,2-dimethyl-1,3-dioxolan-4-yl)ethyl)-2-((2-(2-methoxyphenyl)pyrimidin-4-yl)methoxy)phenyl)propanoate

To a solution of Example 127B (1.8 g) in tetrahydrofuran (10 mL) was added Pd/C (10%, 0.2 g). The mixture was stirred under hydrogen (50 psi) for 6 hours. The mixture was filtered and concentrated under vacuum to give the title compound. MS (ESI) m/z 579.4 (M+H)⁺.

### Example 127D

### (2R)-ethyl 3-(5-(2-(2,2-dimethyl-1,3-dioxolan-4-yl)ethyl)-2-((2-(2-methoxyphenyl)pyrimidin-4-yl)methoxy)phenyl)-2-hydroxypropanoate

To a solution of Example 127C (0.592 g) in ethanol (20 mL) was added K₂CO₃ (0.72 g). The mixture was stirred at room temperature for 1 hour. The mixture was diluted with ethyl acetate (400 mL), washed with water and brine, dried over Na₂SO₄, and filtered. Evaporation of the solvent provided the title compound. MS (ESI) m/z 537.3 (M+H)⁺.

### Example 127E

### (R)-ethyl 2-((5-bromo-6-(4-fluorophenyl)thieno[2,3-d]pyrimidin-4-yl)oxy)-3-(5-(2-((S)-2,2-dimethyl-1,3-dioxolan-4-yl)ethyl)-2-((2-(2-methoxyphenyl)pyrimidin-4-yl)methoxy)phenyl)propanoate

To a solution of Example 127D (500 mg) and Example 1D (384 mg) in t-butanol (20 mL) was added Cs₂CO₃ (911 mg). The reaction mixture was stirred at 65° C. for 3 hours. The mixture was concentrated under vacuum. The residue was dissolved in ethyl acetate (300 mL), washed with water and brine, dried over Na₂SO₄, and filtered. Evaporation of the solvent and column purification of the crude material (20% ethyl acetate in dichloromethane) provided the title compound. MS (ESI) m/z 845.1 (M+H)⁺.

### Example 127F

### (R)-ethyl 2-((5-bromo-6-(4-fluorophenyl)thieno[2,3-d]pyrimidin-4-yl)oxy)-3-(5-((S)-3,4-dihydroxybutyl)-2-((2-(2-methoxyphenyl)pyrimidin-4-yl)methoxy)phenyl)propanoate

To a solution of Example 127E (717 mg) in tetrahydrofuran (10 mL) was added 1N aqueous HCl (10 mL). The reaction mixture was stirred at room temperature overnight. The mixture was concentrated under vacuum and the residue was taken up in ethyl acetate (300 mL) and aqueous Na₂CO₃ (50 mL). The organic layer was washed with brine and dried over Na₂SO₄. Filtration, and evaporation of the solvent provided the title compound. MS (ESI) m/z 803.3 (M+H)⁺.

### Example 127G

### (R)-ethyl 2-((5-bromo-6-(4-fluorophenyl)thieno[2,3-d]pyrimidin-4-yl)oxy)-3-(5-((S)-3-hydroxy-4-(tosyloxy)butyl)-2-((2-(2-methoxyphenyl)pyrimidin-4-yl)methoxy)phenyl)propanoate

To a solution of Example 127F (163 mg) in dichloromethane (10 mL) at 0° C. was added triethylamine (0.8 mL) followed by a solution of para-toluenesulfonic acid monohydrate (46.5 mg) in dichloromethane (2 mL), and the reaction was allowed to stir at room temperature overnight. The reaction mixture was diluted with ethyl acetate (200 mL) and saturated aqueous NaHCO₃. The aqueous layer was extracted three times with ethyl acetate, and the combined organic layers were dried (Na₂SO₄), filtered and concentrated. The residue was purified by column chromatography (20% ethyl acetate in dichloromethane) to give the title compound. MS (ESI) m/z 958.9 (M+H)⁺.

### Example 127H

### ethyl (R)-2-((5-(3,5-dichloro-4-hydroxyphenyl)-6-(4-fluorophenyl)thieno[2,3-d]pyrimidin-4-yl)oxy)-3-(5-((S)-3-hydroxy-4-(tosyloxy)butyl)-2-((2-(2-methoxyphenyl)pyrimidin-4-yl)methoxy)phenyl)propanoate

(3,5-Dichloro-4-hydroxyphenyl)boronic acid (19 mg), Example 127G (88 mg), bis(di-tert-butyl(4-dimethylaminophenyl)phosphine)dichloropalladium(II) (13.01 mg) and K₃PO₄ (58.5 mg) were placed in 20 mL vial. Tetrahydrofuran (10 mL) and water (5 mL) were added. The reaction mixture was purged with argon for 3 minutes. The reaction mixture was stirred at room temperature 3 hours. The mixture was diluted with ethyl acetate (300 mL), washed with water and brine, dried over Na₂SO₄, and filtered. Evaporation of the solvent gave the crude product which was used without further purification. MS (ESI) m/z 1040.2 (M+H)⁺.

### Example 127I

### ethyl (7R,16R)-19,23-dichloro-1-(4-fluorophenyl)-10-{[2-(2-methoxyphenyl)pyrimidin-4-yl]methoxy}-16-{[(4-methylbenzene-1-sulfonyl)oxy]methyl}-7,8,15,16-tetrahydro-14H-18,21-etheno-13,9-(metheno)-6,17-dioxa-2-thia-3,5-diazacyclononadeca[1,2,3-cd]indene-7-carboxylate

To a solution of Example 127G (114 mg) in dichloromethane (3 mL) was added tetrakis(triphenylphosphine)palladium(0) (34.5 mg) and di-tert-butyl azodicarboxylate (30.3 mg). The mixture was stirred at 40° C. for 1.5 hours. The mixture was loaded on a column (25 g Grace) and eluted with 20% ethyl acetate in dichloromethane to give the title compound. MS (ESI) m/z 1023.2 (M+H)⁺.

### Example 127J

### ethyl (7R,16R)-19,23-dichloro-1-(4-fluorophenyl)-10-{[2-(2-methoxyphenyl)pyrimidin-4-yl]methoxy}-16-[(4-methylpiperazin-1-yl)methyl]-7,8,15,16-tetrahydro-14H-18,21-etheno-13,9-(metheno)-6,17-dioxa-2-thia-3,5-diazacyclononadeca[1,2,3-cd]indene-7-carboxylate

To a solution of Example 127I (69.2 mg) in N,N-dimethylformamide (1 mL) was added 1-methylpiperazine (203 mg). The reaction was stirred at 65° C. overnight. The mixture was diluted with ethyl acetate (100 mL), washed with water and brine, dried over Na₂SO₄, and filtered. Evaporation of the solvent provided the title compound. MS (ESI) m/z 951.1 (M+H)⁺.

### Example 127K

### (7R,16R)-19,23-dichloro-1-(4-fluorophenyl)-10-{[2-(2-methoxyphenyl)pyrimidin-4-yl]methoxy}-16-[(4-methylpiperazin-1-yl)methyl]-7,8,15,16-tetrahydro-14H-18,21-etheno-9,13-(metheno)-6,17-dioxa-2-thia-3,5-diazacyclononadeca[1,2,3-cd]indene-7-carboxylic acid

The title compound was prepared as described in Example 10F, substituting Example 127J for Example 10E. ¹H NMR (400 MHz, dimethyl sulfoxide-d₆) δ ppm 8.88 (d, 1H), 8.65 (s, 1H), 8.59 (d, 2H), 7.91 (d, 1H), 7.64 (d, 1H), 7.53-7.40 (m, 5H), 7.32-7.22 (m, 2H), 7.18-7.07 (m, 3H), 7.06-6.89 (m, 4H), 6.30 (d, 1H), 5.80-5.67 (m, 1H), 5.32-5.14 (m, 2H), 4.88-4.70 (m, 1H), 3.74 (s, 31H), 3.17-2.88 (m, 4H), 2.79 (s, 3H), 2.42 (dt, 1H), 1.92 (p, J=5.5 Hz, 2H). MS (ESI) m/z 921.3 (M+H)⁺.

### Example 128

### (7S,16R)-19,23-dichloro-1-(4-fluorophenyl)-10-{[2-(2-methoxyphenyl)pyrimidin-4-yl]methoxy}-16-[(4-methylpiperazin-1-yl)methyl]-7,8,15,16-tetrahydro-14H-18,21-etheno-9,13-(metheno)-6,17-dioxa-2-thia-3,5-diazacyclononadeca[1,2,3-cd]indene-7-carboxylic acid

The title compound was isolated as a minor product from Example 127K. ¹H NMR (501 MHz, dimethyl sulfoxide-d₆) δ ppm 8.89 (d, 1H), 8.73 (s, 1H), 7.89 (d, 1H), 7.56 (dd, 1H), 7.52-7.44 (m, 2H), 7.38-7.32 (m, 2H), 7.30-7.23 (m, 3H), 7.17 (dd, 1H), 7.08 (dd, 1H), 6.95 (dd, 1H), 6.89 (d, 1H), 6.08 (d, 1H), 6.00 (dd, 1H), 5.17 (s, 2H), 4.24 (d, 1H), 3.94 (dd, 1H), 3.78 (s, 3H), 3.32 (d, 1H), 3.19-2.89 (m, 4H), 2.76 (s, 3H), 2.70-2.55 (m, 1H), 2.17-1.98 (m, 3H). MS (ESI) m/z 921.3 (M+H)⁺.

### Example 129

### (7R,16R)-19,23-dichloro-1-(4-fluorophenyl)-10-{[2-(2-methoxyphenyl)pyrimidin-4-yl]methoxy}-16-[(4-methylpiperazin-1-yl)methyl]-7,8,15,16-tetrahydro-18,21-etheno-9,13-(metheno)-2,6,14,17-tetraoxa-3,5-diazacyclononadeca[1,2,3-cd]indene-7-carboxylic acid

### Example 129A

### (S)-3-(bis(4-methoxyphenyl)(phenyl)methoxy)-2-(4-bromo-2,6-dichlorophenoxy)propyl 4-methylbenzenesulfonate

Example 112B (2.0 g), 4-bromo-2,6 dichlorophenol (1.06 g) and triphenylphosphine (1.43 g) were mixed under argon. Tetrahydrofuran (15 mL) was added followed by di-tert-butyl azodicarboxylate (1.26 g). The reaction mixture was heated to 55° C. for 4 hours. After addition of more triphenylphosphine (143 mg) and di-tert-butyl azodicarboxylate (125 mg), the stirring was continued for an additional 1.5 hours at 55° C. The solvent was removed in vacuo, the residue obtained was treated with cyclohexane, and the mixture stirred for 2 hours at room temperature. The material was filtered off and washed with cyclohexane. The filtrate and some gummy material left in the reaction flask were combined, dried in vacuo and purified by silica gel flash chromatography (120 g Grace Reveleris column, gradient ethyl acetate in heptane 2-50%) to give the title compound which was directly used in the next step.

### Example 129B

### (R)-1-(3-(bis(4-methoxyphenyl)(phenyl)methoxy)-2-(4-bromo-2,6-dichlorophenoxy)propyl)-4-methylpiperazine

A solution of Example 129A (2.21 g), 1-methylpiperazine (1.43 g) and triethylamine (0.87 mg) in N,N-dimethylformamide (20 mL) was heated to 85° C. overnight. Water was added and the mixture was extracted with ethyl acetate. The combined organic layers were washed with water, dried over MgSO₄, filtered, and concentrated in vacuo. The residue obtained was purified by silica gel flash chromatography (40 g Grace Reveleris column, gradient ethyl acetate/ethanol (2:1) in heptane 2-100%) to give the title compound. MS (ESI) m/z 397.0 ([M−DMTr]+2H)⁺.

### Example 129C

### (R)-1-(3-(bis(4-methoxyphenyl)(phenyl)methoxy)-2-(2,6-dichloro-4-(4,4,5,5-tetramethyl-1,3,2-dioxaborolan-2-yl)phenoxy)propyl)-4-methylpiperazine

A solution of Example 129B (1000 mg) in 2-methyltetrahydrofuran (14 mL) was degassed and added to a mixture of potassium acetate (280 mg, dried at 100° C.), 1,1′-bis(diphenylphosphino)ferrocene-palladium (II) dichloride dichloromethane complex (58 mg) and bis(pinacolato)diboron (435 mg). The reaction mixture was heated for 14 hours at 90° C. Dilution with ethyl acetate followed by filtration (diatomaceous earth) and removal of the solvent in vacuo provided the crude product which was purified by silica gel flash chromatography (40 g Grace Reveleris column, gradient ethyl acetate/ethanol (2:1) in heptane 2-100%) to provide the title compound. MS (ESI) m/z 445.1 ([M−DMTr]+2H)⁺.

### Example 129D

### (R)-ethyl 2-((5-(4-(((S)-1-(bis(4-methoxyphenyl)(phenyl)methoxy)-3-(4-methylpiperazin-1-yl)propan-2-yl)oxy)-3,5-dichlorophenyl)-6-(4-fluorophenyl)furo[2,3-d]pyrimidin-4-yl)oxy)-3-(5-((tert-butyldimethylsilyl)oxy)-2-((2-(2-methoxyphenyl)pyrimidin-4-yl)methoxy)phenyl)propanoate

A mixture of Example 68C (100.0 mg), Example 129C (113.0 mg), cesium carbonate (118.0 mg) and bis (di-tert-butyl(4-dimethylaminophenyl)phosphine)dichloropalladium (II) (8.5 mg) were stirred under argon. A solution of tetrahydrofuran (2.4 mL) and water (0.6 mL) was degassed and added to the reaction mixture. After stirring for 4 days at room temperature, water was added and the mixture was extracted with ethyl acetate. The combined organic layers were washed with water, dried over MgSO₄, filtered, and concentrated in vacuo. The residue obtained was purified by silica gel flash chromatography (12 g Grace Reveleris column, gradient methanol in dichloromethane 1-10%) to give the title compound. MS (ESI) m/z 1067.4 ([M−DMT]+2H)⁺.

### Example 129E

### (R)-ethyl 3-(5-((tert-butyldimethylsilyl)oxy)-2-((2-(2-methoxyphenyl)pyrimidin-4-yl)methoxy)phenyl)-2-((5-(3,5-dichloro-4-(((S)-1-hydroxy-3-(4-methylpiperazin-1-yl)propan-2-yl)oxy)phenyl)-6-(4-fluorophenyl)furo[2,3-d]pyrimidin-4-yl)oxy)propanoate

Formic acid (544 mg) was added to a solution of Example 129D (180 mg) in dichloromethane/methanol (0.8 mL/0.8 mL) and the reaction mixture was stirred for 5 hours at room temperature. The pH was adjusted to 9 under ice-cooling using saturated aqueous NaHCO₃ solution. After extraction with ethyl acetate, the combined organic layers were washed with water, dried over MgSO₄, filtered, and concentrated in vacuo. The residue obtained was purified by silica gel flash chromatography (12 g Grace Reveleris column, gradient methanol in dichloromethane 1-10%) to give the title compound. MS (ESI) m/z 1067.3.2 (M+H)⁺.

### Example 129F

### (R)-ethyl 2-((5-(3,5-dichloro-4-(((S)-1-hydroxy-3-(4-methylpiperazin-1-yl)propan-2-yl)oxy)phenyl)-6-(4-fluorophenyl)furo[2,3-d]pyrimidin-4-yl)oxy)-3-(5-hydroxy-2-((2-(2-methoxyphenyl)pyrimidin-4-yl)methoxy)phenyl)propanoate

TBAF (tetrabutyl ammonium fluoride, 0.28 mL, 1M solution in tetrahydrofuran) was added to a solution of Example 129E (100 mg) in tetrahydrofuran (2 mL). After stirring for 25 minutes at room temperature, aqueous ammonium chloride solution (10%) was added and the mixture was extracted with ethyl acetate. The combined extracts were washed with water, dried over MgSO₄, filtered, and the solvent was reduced in vacuo. The residue obtained was purified by silica gel flash chromatography (4 g Grace Reveleris column, gradient methanol in dichloromethane 1-15%) to give the title compound. MS (ESI) m/z 953.2 (M+H)⁺.

### Example 129G

### ethyl (7R,16R)-19,23-dichloro-1-(4-fluorophenyl)-10-{[2-(2-methoxyphenyl)pyrimidin-4-yl]methoxy}-16-[(4-methylpiperazin-1-yl)methyl]-7,8,15,16-tetrahydro-18,21-etheno-9,13-(metheno)-2,6,14,17-tetraoxa-3,5-diazacyclononadeca[1,2,3-cd]indene-7-carboxylate

Example 129F (25.0 mg), triphenylphosphine (20.6 mg) and di-tert-butyl azodicarboxylate (18.1 mg) were mixed in a microwave vial under argon atmosphere. Tetrahydrofuran (5 mL) was added and the mixture obtained was stirred overnight at room temperature. After heating for 4 hours at 50° C., the solvent was removed in vacuo. Purification by HPLC (xBridge prepMS C18 19×150 mm 5 m column, gradient 5-100% acetonitrile+0.1% trifluoroacetic acid in water+0.1% trifluoroacetic acid over 11 minutes, retention time 5.3 minutes) provided the title compound. MS (ESI) m/z 935.4 (M+H)⁺.

### Example 129H

### (7R,16R)-19,23-dichloro-1-(4-fluorophenyl)-10-{[2-(2-methoxyphenyl)pyrimidin-4-yl]methoxy}-16-[(4-methylpiperazin-1-yl)methyl]-7,8,15,16-tetrahydro-18,21-etheno-9,13-(metheno)-2,6,14,17-tetraoxa-3,5-diazacyclononadeca[1,2,3-cd]indene-7-carboxylic acid

A solution of LiOH (9.0 mg) in water (0.2 mL) was added to a solution of Example 129G (22 mg) in methanol/water (0.2 mL/0.2 mL). The reaction mixture was stirred overnight at room temperature. After addition of trifluoroacetic acid (53.9 mg) the solvent was removed in vacuo. Purification by HPLC (Waters X-Bridge C18 19×150 mm 5 m column, gradient 5-100% acetonitrile+0.1% trifluoroacetic acid in water+0.1% trifluoroacetic acid over 11 minutes, retention time 5.6 minutes) provided the title compound. ¹H NMR (600 MHz, dimethyl sulfoxide-d₆,) δ ppm 13.15 (s, 1H), 9.37 (s, 1H), 8.90 (d, 1H), 8.64 (s, 1H), 7.71 (d, 1H), 7.60 (d, 1H), 7.57-7.51 (m, 3H), 7.49-7.45 (m, 2H), 7.34-7.30 (m, 2H), 7.16 (d, 1H), 7.06 (t, 1H), 6.92 (d, 1H), 6.76 (dd, 1H), 6.23 (d, 1H), 6.17 (dd, 1H), 5.21-5.13 (m, 2H), 5.07-5.03 (m, 1H), 4.39-4.33 (m, 1H), 4.29-4.25 (m, 1H), 3.77 (s, 3H), 3.75-3.29 (broad m, 3H), 3.27-3.22 (m, 2H), 3.14-3.03 (broad m, 5H), 2.97-2.85 (m, 2H), 2.81 (s, 3H). MS (ESI) m/z 907.4 (M+H)⁺.

### Example 130

### (7R,20S)-18-chloro-1-(4-fluorophenyl)-10-({2-[2-(methanesulfonyl)phenyl]pyrimidin-4-yl}methoxy)-19-methyl-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylic acid

### Example 130A

### 2-(methylsulfonyl)benzimidamide

To a mixture of ammonium chloride (11.22 g) in toluene (100 mL) was added trimethylaluminum (105 mL, 2M in toluene) slowly at 0° C. under nitrogen until there was no further evolution of gas. Next, 2-(methylsulfonyl)benzonitrile (10 g) was added and the reaction mixture was stirred at 100° C. for 12 hours. The combined mixture was cooled to 0° C., quenched carefully with 50 mL methanol, and stirred at 20° C. for 2 hours. The material was filtered and washed with methanol several times. The filtrate was concentrated under vacuum to give the title compound. ¹H NMR (400 MHz, dimethyl sulfoxide-d₆) δ ppm 8.97 (br s, 3H), 8.12 (m, 1H), 7.90 (m, 2H), 7.71 (m, 1H), 3.37 (s, 3H).

### Example 130B

### 4-(dimethoxymethyl)-2-(2-(methylsulfonyl)phenyl)pyrimidine

To a mixture of Example 130A (10 g) in methanol (50 mL) was added sodium methanolate (45.4 mLl, 2M in methanol) and Example 100A (9.93 g). The reaction mixture was stirred at 80° C. for 12 hours. The mixture was concentrated, diluted with water (50 mL), and extracted with ethyl acetate (2×50 mL). The combined organic layers were dried over Na₂SO₄, filtered and concentrated to give a residue which was purified by column chromatography on silica gel (petroleum ether/ethyl acetate=20:1 to 2:1) to give the title compound. ¹H NMR (400 MHz, dimethyl sulfoxide-d₆) δ ppm 9.00 (d, 1H), 8.10 (d, 1H), 7.88 (m, 1H), 7.78 (m, 2H), 7.60 (d, 1H), 5.41 (s, 1H), 3.59 (s, 3H), 3.33 (s, 6H).

### Example 130C

### (2-(2-(methylsulfonyl)phenyl)pyrimidin-4-yl)methanol

To a mixture of Example 130B (7.5 g) in dioxane (52 mL) was added 4 M aqueous hydrogen chloride (52.0 mL) at 25° C. The reaction mixture was stirred at 60° C. for 12 hours. The pH of the reaction mixture was adjusted to 8 by addition of saturated sodium hydroxide solution. To this mixture was added sodium borohydride (1.748 g) at 0° C. The reaction mixture was stirred at 0° C. for 2 hours. The mixture was extracted with ethyl acetate (3×300 mL). The combined organic layers were washed with brine (300 mL), dried over Na₂SO₄, filtered and concentrated under vacuum to afford a residue which was chromatographed on silica gel (petroleum ether/ethyl acetate 10:1-1:1) to give the title compound. ¹H NMR (400 MHz, chloroform-d) δ ppm 8.78 (d, J=5.1 Hz, 1H), 8.20 (d, J=8.4 Hz, 1H), 7.82-7.72 (m, 2H), 7.71-7.63 (m, 1H), 7.38 (d, J=5.1 Hz, 1H), 4.82 (d, J=5.3 Hz, 2H), 3.51 (s, 3H), 3.22 (t, J=5.5 Hz, 1H).

### Example 130D

### 4-(chloromethyl)-2-(2-(methylsulfonyl)phenyl)pyrimidine

To a solution of Example 130C (256 mg) in dioxane (6 mL) was added (chloromethylene)dimethyliminium chloride (160 mg). The mixture was stirred at room temperature for 45 minutes. The mixture was diluted with ethyl acetate (100 mL), washed with aqueous NaHCO₃, water, and brine, dried over Na₂SO₄, and filtered. Evaporation of the solvent and column (24 g Grace) purification (20% ethyl acetate in heptane) provided the title compound. MS (ESI) m/e 283.1 (M+H)⁺.

### Example 130E

### ethyl (7R,20S)-18-chloro-1-(4-fluorophenyl)-10-({2-[2-(methanesulfonyl)phenyl]pyrimidin-4-yl}methoxy)-19-methyl-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylate

The title compound was prepared as described in Example 65N, substituting Example 130D for Example 65E. MS (ESI) m/e 976.2 (M+H)⁺.

### Example 130F

### (7R,20S)-18-chloro-1-(4-fluorophenyl)-10-({2-[2-(methanesulfonyl)phenyl]pyrimidin-4-yl}methoxy)-19-methyl-15-[2-(4-methylpiperazin-1-yl)ethyl]-7,8,15,16-tetrahydro-14H-17,20-etheno-13,9-(metheno)-6-oxa-2-thia-3,5,15-triazacyclooctadeca[1,2,3-cd]indene-7-carboxylic acid

The title compound was prepared as described in Example 10F, substituting Example 130E for Example 10E. ¹H NMR (501 MHz, dimethyl sulfoxide-d₆) δ ppm 9.14 (d, 1H), 8.92 (d, 1H), 8.80 (d, 1H), 8.75-8.57 (m, 2H), 8.17-8.05 (m, 2H), 7.94-7.70 (m, 9H), 7.59-7.52 (m, 1H), 7.40-7.09 (m, 10H), 6.70-6.49 (m, 1H), 6.01-5.90 (m, 2H), 5.31-5.14 (m, 1H), 4.89 (s, 2H), 3.19 (s, 3H), 3.09-2.96 (m, 2H), 2.80 (s, 1H), 1.80 (s, 3H). MS (ESI) m/e 948.3 (M+H)⁺.

### Example 131

### (7R,20S)-18-chloro-1-(4-fluorophenyl)-19-methyl-15-[2-(4-methylpiperazin-1-yl)ethyl]-10-({2-[(3R)-oxolan-3-yl]pyrimidin-4-yl}methoxy)-7,8,15,16-tetrahydro-14H-17,20-etheno-13,9-(metheno)-6-oxa-2-thia-3,5,15-triazacyclooctadeca[1,2,3-cd]indene-7-carboxylic acid

### Example 131A

### tetrahydrofuran-3-carboxamide

Tetrahydrofuran-3-carboxylic acid (15 g) was dissolved in tetrahydrofuran (300 mL), and cooled to 3° C. using an ice-water bath. 1,1′-Carbonyldiimidazole (25 g) was added all at once. The reaction was stirred cold for five minutes, and the bath was removed and stirring was continued at room temperature for two hours. The reaction was cooled using an ice-water bath for 15 minutes, and concentrated ammonium hydroxide (25 mL) was added. The reaction mixture was stirred cold for one hour, then at room temperature for one hour. The reaction mixture was concentrated and partitioned between ethyl acetate (150 mL) and 6 N aqueous HCl (40 mL). The layers were separated, and the aqueous layer was extracted with ethyl acetate (4×200 mL). The combined ethyl acetate layers were dried over sodium sulfate, filtered, and concentrated. The crude product was carried on with no purification. MS (DCI) m/z 134.0 (M+H)⁺.

### Example 131B

### methyl tetrahydrofuran-3-carbimidate

Example 131A (7.0 g) was added to dichloromethane (190 mL), and cooled using an ice-water bath for 15 minutes. Trimethyloxonium tetrafluoroborate (10.0 g) was added all at once. The reaction was allowed to come to room temperature overnight. Saturated aqueous sodium bicarbonate (240 mL) was added and the layers were separated. The aqueous layer was extracted with ethyl acetate (3×150 mL). The combined ethyl acetate layers were dried over sodium sulfate, filtered, and concentrated. The crude product was carried on with no purification.

### Example 131C

### tetrahydrofuran-3-carboximidamide, hydrochloride salt

Example 131B (6.1 g) was dissolved in methanol (140 mL), and cooled using an ice-water bath for 15 minutes. Ammonium hydrochloride (3.8 g) was added all at once. The reaction was stirred cold for five minutes, at room temperature for 30 minutes, and finally at 70° C. overnight. The reaction was cooled and concentrated, and the residue was dried under high vacuum for one hour. The residue was vigorously shaken in dichloromethane/methanol 30/1 (45 mL) for 10 minutes, and filtered through diatomaceous earth. The filtrate was concentrated to give the title compound that was carried on with no further purification. MS (DCI) m/z 114.9 (M+H)⁺.

### Example 131D

### 4-(dimethoxymethyl)-2-(tetrahydrofuran-3-yl)pyrimidine

The title compound was prepared by substituting Example 131C for Example 65B in Example 65C. MS (DCI) m/z 225.0 (M+H)⁺.

### Example 131E

### (2-(tetrahydrofuran-3-yl)pyrimidin-4-yl)methanol

The title compound was prepared by substituting Example 131D for Example 65C in Example 65D. MS (DCI) m/z 181.0 (M+H)⁺.

### Example 131F

### (R*)-(2-(tetrahydrofuran-3-yl)pyrimidin-4-yl)methanol

Example 131E (1.5 g) was subjected to supercritical fluid chromatography: 21×250 mm (5) YMC Amylose-C column, 25% isopropanol in supercritical carbon dioxide, 60 mL/minute, 3.5 minutes total time. The title compound had a retention time of 1.98 minutes. The absolute stereochemistry was arbitrarily assigned. MS (DCI) m/z 181.0 (M+H)⁺.

### Example 131G

### (S*)-(2-(tetrahydrofuran-3-yl)pyrimidin-4-yl)methanol

The title compound was obtained via chromatography as described in Example 131F. The title compound had a retention time of 2.59 minutes. The absolute stereochemistry was arbitrarily assigned. MS (DCI) m/z 181.0 (M+H)⁺.

### Example 131H

### (R*)-4-(chloromethyl)-2-(tetrahydrofuran-3-yl)pyrimidine

The title compound was prepared by substituting Example 131F for Example 65D in Example 65E. MS (DCI) m/z 199.0 (M+H)⁺.

### Example 131I

### ethyl (7R,20S)-18-chloro-1-(4-fluorophenyl)-19-methyl-15-[2-(4-methylpiperazin-1-yl)ethyl]-10-({2-[(3R*)-oxolan-3-yl]pyrimidin-4-yl}methoxy)-7,8,15,16-tetrahydro-14H-17,20-etheno-13,9-(metheno)-6-oxa-2-thia-3,5,15-triazacyclooctadeca[1,2,3-cd]indene-7-carboxylate

Example 65M (50 mg) and Example 131H (27 mg) were dissolved in dimethylformamide (0.25 mL), and cesium carbonate (70 mg) was added. The reaction mixture was stirred at room temperature overnight. The reaction mixture was diluted with dimethylformamide (1 mL), followed by the addition of acetic acid (0.12 mL) and water (0.1 mL). Purification was by preparative LC: 250×50 mm Luna column using 10-80% acetonitrile in 0.1% aqueous trifluoroacetic acid over 30 minutes. Product-containing fractions were lyophilized to provide the title compound. MS (ESI) m/z 892.2 (M+H)⁺.

### Example 131J

### (7R,20S)-18-chloro-1-(4-fluorophenyl)-19-methyl-15-[2-(4-methylpiperazin-1-yl)ethyl]-10-({2-[(3R*)-oxolan-3-yl]pyrimidin-4-yl}methoxy)-7,8,15,16-tetrahydro-14H-17,20-etheno-13,9-(metheno)-6-oxa-2-thia-3,5,15-triazacyclooctadeca[1,2,3-cd]indene-7-carboxylic acid

The title compound was prepared by substituting Example 131I for Example 65N in Example 65O. ¹H NMR (500 MHz, dimethylsulfoxide-d₆) δ ppm 8.65 (s, 1H), 8.56 (d, 1H), 7.53 (d, 1H), 7.29 (d, 1H), 7.23 (m, 2H), 7.16 (br s, 1H), 7.13 (m, 3H), 6.83 (d, 1H), 6.51 (s, 1H), 5.94 (dd, 1H), 5.15 (d, 1H), 5.00 (d, 1H), 4.36 (v br s, 2H), 4.18 (br s, 2H), 4.08 (m, 1H), 3.83 (m, 4H), 3.61 (m, 6H), 3.20 (m, 4H), 3.06 (m, 4H), 2.81 (s, 3H), 2.23 (m, 2H) 1.72 (s, 3H). MS (ESI) m/z 864.3 (M+H)⁺.

### Example 132

### (7R,20S)-18-chloro-1-(4-fluorophenyl)-19-methyl-15-[2-(4-methylpiperazin-1-yl)ethyl]-10-({2-[(3S)-oxolan-3-yl]pyrimidin-4-yl}methoxy)-7,8,15,16-tetrahydro-14H-17,20-etheno-13,9-(metheno)-6-oxa-2-thia-3,5,15-triazacyclooctadeca[1,2,3-cd]indene-7-carboxylic acid

### Example 132A

### (S*)-4-(chloromethyl)-2-(tetrahydrofuran-3-yl)pyrimidine

The title compound was prepared by substituting Example 131G for Example 65D in Example 65E. MS (DCI) m/z 199.0 (M+H)⁺.

### Example 132B

### ethyl (7R,20S)-18-chloro-1-(4-fluorophenyl)-19-methyl-15-[2-(4-methylpiperazin-1-yl)ethyl]-10-({2-[(3S*)-oxolan-3-yl]pyrimidin-4-yl}methoxy)-7,8,15,16-tetrahydro-14H-17,20-etheno-13,9-(metheno)-6-oxa-2-thia-3,5,15-triazacyclooctadeca[1,2,3-cd]indene-7-carboxylate

The title compound was prepared by substituting Example 132A for Example 131H in Example 131I. MS (ESI) m/z 892.3 (M+1).

### Example 132C

### (7R,20S)-18-chloro-1-(4-fluorophenyl)-19-methyl-15-[2-(4-methylpiperazin-1-yl)ethyl]-10-({2-[(3S*)-oxolan-3-yl]pyrimidin-4-yl}methoxy)-7,8,15,16-tetrahydro-14H-17,20-etheno-13,9-(metheno)-6-oxa-2-thia-3,5,15-triazacyclooctadeca[1,2,3-cd]indene-7-carboxylic acid

The title compound was prepared by substituting Example 132B for Example 65N in Example 65O. ¹H NMR (500 MHz, dimethylsulfoxide-d₆) δ ppm 8.63 (s, 1H), 8.55 (d, 1H), 7.50 (d, 1H), 7.28 (d, 1H), 7.22 (m, 2H), 7.18 (br s, 1H), 7.14 (m, 3H), 6.80 (d, 1H), 6.50 (s, 1H), 5.92 (dd, 1H), 5.15 (d, 1H), 4.98 (d, 1H), 4.29 (v br s, 2H), 4.12 (br s, 2H), 4.06 (m, 1H), 3.83 (m, 4H), 3.61 (m, 6H), 3.19 (m, 4H), 3.11 (m, 4H), 2.79 (s, 3H), 2.23 (m, 2H) 1.71 (s, 3H). MS (ESI) m/z 864.3 (M+H)⁺.

### Example 133

### (7R,16R,21S)-19-chloro-16-{[(3R)-3,4-dimethylpiperazin-1-yl]methyl}-1-(4-fluorophenyl)-10-{[2-(2-methoxyphenyl)pyrimidin-4-yl]methoxy}-20-methyl-7,8,15,16-tetrahydro-18,21-etheno-13,9-(metheno)-6,14,17-trioxa-2-thia-3,5-diazacyclononadeca[1,2,3-cd]indene-7-carboxylic acid

### Example 133A

### ethyl (7R,16R,21S)-19-chloro-16-{[(3R)-3,4-dimethylpiperazin-1-yl]methyl}-1-(4-fluorophenyl)-10-{[2-(2-methoxyphenyl)pyrimidin-4-yl]methoxy}-20-methyl-7,8,15,16-tetrahydro-18,21-etheno-13,9-(metheno)-6,14,17-trioxa-2-thia-3,5-diazacyclononadeca[1,2,3-cd]indene-7-carboxylate

A 4 mL vial was charged with Example 731 (60 mg), (2R)-1,2-dimethylpiperazine (109 mg) and dimethylformamide (0.15 mL). The vial was capped and stirred at 45° C. for 19 hours. To the mixture was added 2 mL of water. The precipitate obtained was sonicated for a few minutes, filtered and washed with 2 mL of water. The material was collected and dried under high vacuum to afford the title compound. MS (ESI) m/z 945.3 (M+H)⁺.

### Example 133B

### (7R,16R,21S)-19-chloro-16-{[(3R)-3,4-dimethylpiperazin-1-yl]methyl}-1-(4-fluorophenyl)-10-{[2-(2-methoxyphenyl)pyrimidin-4-yl]methoxy}-20-methyl-7,8,15,16-tetrahydro-18,21-etheno-13,9-(metheno)-6,14,17-trioxa-2-thia-3,5-diazacyclononadeca[1,2,3-cd]indene-7-carboxylic acid

To a solution of Example 133A (50 mg) in tetrahydrofuran (0.53 mL) and methanol (0.265 mL) was slowly added LiOH solution (1.0 M in H₂O, 0.53 mL). The mixture was stirred for one day. The reaction mixture was acidified at 0° C. with acetic acid and was purified on a Gilson prep HPLC (Zorbax, C-18, 250×21.2 mm column, 5-75% acetonitrile in water (0.1% trifluoroacetic acid)) to give the title compound after lyophilization. ¹H NMR (400 MHz, dimethyl sulfoxide-d₆) δ ppm 9.45 (s, 1H), 8.89 (d, J=5.1 Hz, 1H), 8.75 (s, 1H), 7.58-7.51 (m, 2H), 7.47 (td, J=7.9, 1.8 Hz, 1H), 7.26-7.12 (m, 6H), 7.10-7.03 (m, 1H), 6.97 (d, J=8.3 Hz, 1H), 6.91 (d, J=9.0 Hz, 1H), 6.84 (dd, J=9.0, 2.8 Hz, 1H), 6.16 (d, J=4.8 Hz, 1H), 5.66 (s, 1H), 5.18 (q, J=15.0 Hz, 2H), 4.64-4.29 (m, 4H), 3.90-3.83 (m, 2H), 3.77 (s, 3H), 3.45-2.99 (m, 4H), 2.90 (d, J=15.7 Hz, 2H), 2.80 (s, 3H), 2.71 (d, J=5.8 Hz, 2H), 2.24 (s, 3H). MS (ESI) m/z 917.4 (M+H)⁺.

### Example 134

### (7R,16S,21S)-19-chloro-1-(4-fluorophenyl)-10-{[2-(2-methoxyphenyl)pyrimidin-4-yl]methoxy}-20-methyl-16-[(4-methylpiperazin-1-yl)methyl]-7,8,15,16-tetrahydro-14H-18,21-etheno-9,13-(metheno)-6,17-dioxa-2-thia-3,5-diazacyclononadeca[1,2,3-cd]indene-7-carboxylic acid

### Example 134A

### (R)-ethyl 2-((5-bromo-6-(4-fluorophenyl)thieno[2,3-d]pyrimidin-4-yl)oxy)-3-(5-((S)-4-((tert-butyldiphenylsilyl)oxy)-3-hydroxybutyl)-2-((2-(2-methoxyphenyl)pyrimidin-4-yl)methoxy)phenyl)propanoate

To a solution of Example 127F (470 mg) in N,N-dimethylformamide (10 mL) was added imidazole (80 mg), and tert-butylchlorodiphenylsilane (193 mg). The reaction mixture was stirred at ambient temperature overnight. The mixture was diluted with ethyl acetate, and washed with water and brine. The organic layer was dried over Na₂SO₄, filtered, and concentrated. The crude material was loaded on a column and was eluted with 20% ethyl acetate in dichloromethane to give the title compound. MS (ESI) m/z 1043.2 (M+H)⁺.

### Example 134B

### (R)-ethyl 3-(5-((R)-3-acetoxy-4-((tert-butyldiphenylsilyl)oxy)butyl)-2-((2-(2-methoxyphenyl)pyrimidin-4-yl)methoxy)phenyl)-2-((5-bromo-6-(4-fluorophenyl)thieno[2,3-d]pyrimidin-4-yl)oxy)propanoate

To a cooled (0° C.) solution of Example 134A (440 mg) and triphenylphosphine (133 mg) in tetrahydrofuran (10 mL) was added di-tert-butyl azodicarboxylate (117 mg). The reaction mixture was stirred at 0° C. for 5 minutes and acetic acid (36 mg) was added. The mixture was stirred room temperature overnight. The mixture was diluted with ethyl acetate, and washed with water and brine. The organic layer was dried over Na₂SO₄, filtered, and concentrated. The crude material was loaded on a column and was eluted with 20% ethyl acetate in dichloromethane to give the title compound. MS (ESI) m/z 1085.2 (M+H)⁺.

### Example 134C

### (R)-ethyl 2-((5-bromo-6-(4-fluorophenyl)thieno[2,3-d]pyrimidin-4-yl)oxy)-3-(5-((R)-4-((tert-butyldiphenylsilyl)oxy)-3-hydroxybutyl)-2-((2-(2-methoxyphenyl)pyrimidin-4-yl)methoxy)phenyl)propanoate

To a solution of Example 134B (72 mg) in ethanol (1 mL) was added K₂CO₃ (46 mg). The reaction was stirred at room temperature 3 hours. The mixture was diluted with ethyl acetate (100 mL), washed with water and brine, dried over Na₂SO₄, and filtered. Evaporation of the solvent provided the title compound. MS (ESI) m/z 1043.2 (M+H)⁺.

### Example 134D

### (2-chloro-3-methyl-4-(4,4,5,5-tetramethyl-1,3,2-dioxaborolan-2-yl)phenoxy)triisopropylsilane

Example 64B (35.35 g) was taken up in tetrahydrofuran (312 mL) and was cooled to −78° C. (external) under Ar. n-Butyllithium (2.5 M, 41.2 mL) was added dropwise via syringe. The clear solution was stirred for 10 minutes and 2-isopropoxy-4,4,5,5-tetramethyl-1,3,2-dioxaborolane (20.89 g) was added dropwise. The reaction was warmed to room temperature and was stirred overnight. The volatiles were removed by rotary evaporation and the residue was taken up in ethyl acetate and poured into water. The layers were separated and the organics were washed with water and brine. The aqueous layer was back extracted and the combined organics were dried over Na₂SO₄, treated with activated charcoal (to remove pink color), filtered, and concentrated by rotary evaporation. The rotavap was placed under high vacuum and the water bath was set at 80° C. for about an hour. The resulting material was frozen in a dry ice/acetone bath, and methanol was added (25 mL). The mixture was put under high vacuum. The material was triturated at room temperature with methanol again to provide the title compound. MS (ESI) m/z 425.1 (M+H)⁺.

### Example 134E

### (2R)-ethyl 3-(5-((R)-4-((tert-butyldiphenylsilyl)oxy)-3-hydroxybutyl)-2-((2-(2-methoxyphenyl)pyrimidin-4-yl)methoxy)phenyl)-2-((5-((1S)-3-chloro-4-hydroxy-2-methylphenyl)-6-(4-fluorophenyl)thieno[2,3-d]pyrimidin-4-yl)oxy)propanoate

Example 134D (68.5 mg), Example 134C (168 mg), bis(di-tert-butyl(4-dimethylaminophenyl)phosphine)dichloropalladium(II) (23.01 mg) and K₃PO₄ (103 mg) were placed in a 20 mL vial. Tetrahydrofuran (10 mL) and water (5 mL) were added. The reaction mixture was purged with argon for 3 minutes. The reaction mixture was stirred at room temperature 3 hours. The mixture was diluted with ethyl acetate (300 mL), washed with water and brine, dried over Na₂SO₄, and filtered. Evaporation of the solvent gave crude product which was dissolved in N,N-dimethylformamide (5 mL). Potassium acetate (500 mg) was added. The mixture was stirred at room temperature for 3 hours. The mixture was diluted with ethyl acetate (200 mL), washed with saturated aqueous NH₄Cl, water and brine, dried over Na₂SO₄, and filtered. Evaporation of the solvent provided the title compound. MS (ESI) m/z 1103.4 (M+H)⁺.

### Example 134F

### ethyl (7R,16S,21S)-16-({[tert-butyl(diphenyl)silyl]oxy}methyl)-19-chloro-1-(4-fluorophenyl)-10-{[2-(2-methoxyphenyl)pyrimidin-4-yl]methoxy}-20-methyl-7,8,15,16-tetrahydro-14H-18,21-etheno-9,13-(metheno)-6,17-dioxa-2-thia-3,5-diazacyclononadeca[1,2,3-cd]indene-7-carboxylate

To a solution of Example 134E (160 mg) in dichloromethane (10 mL) was added Ph₃P (tetrakis(triphenylphosphine)palladium(0), 45.6 mg) and di-tert-butyl azodicarboxylate (40.1 mg). The mixture was stirred at 40° C. for 1.5 hours. The mixture was loaded on a column (25 g Grace) and was eluted with 20% ethyl acetate in heptane to give the title compound. MS (ESI) m/z 1085.4 (M+H)⁺.

### Example 134G

### ethyl (7R,16S,21S)-19-chloro-1-(4-fluorophenyl)-16-(hydroxymethyl)-10-{[2-(2-methoxyphenyl)pyrimidin-4-yl]methoxy}-20-methyl-7,8,15,16-tetrahydro-14H-18,21-etheno-9,13-(metheno)-6,17-dioxa-2-thia-3,5-diazacyclononadeca[1,2,3-cd]indene-7-carboxylate

To a solution of Example 134F (110 mg) in tetrahydrofuran (5 mL) was added 2 mL of TBAF (tetrabutyl ammonium fluoride, 1 M in tetrahydrofuran, 0.2 mL). The mixture was stirred at room temperature overnight. The mixture was diluted with ethyl acetate (100 mL), washed with water and brine, dried over Na₂SO₄, and filtered. Evaporation of the solvent provided the title compound. MS (ESI) m/z 847.3 (M+H)⁺.

### Example 134H

### ethyl (7R,16S,21S)-19-chloro-1-(4-fluorophenyl)-10-{[2-(2-methoxyphenyl)pyrimidin-4-yl]methoxy}-20-methyl-16-{[(4-methylbenzene-1-sulfonyl)oxy]methyl}-7,8,15,16-tetrahydro-14H-18,21-etheno-9,13-(metheno)-6,17-dioxa-2-thia-3,5-diazacyclononadeca[1,2,3-cd]indene-7-carboxylate

To a solution of Example 134G (80 mg) in dichloromethane (10 mL) was added para-toluenesulfonic acid monohydrate (36 mg) and triethylamine (28.7 mg). The mixture was stirred at room temperature overnight. The mixture was diluted with ethyl acetate (100 mL), washed with water and brine, dried over Na₂SO₄, and filtered. Evaporation of the solvent provided the title compound. MS (ESI) m/z 1001.1 (M+H)⁺.

### Example 134I

### ethyl (7R,16S,21S)-19-chloro-1-(4-fluorophenyl)-10-{[2-(2-methoxyphenyl)pyrimidin-4-yl]methoxy}-20-methyl-16-[(4-methylpiperazin-1-yl)methyl]-7,8,15,16-tetrahydro-14H-18,21-etheno-9,13-(metheno)-6,17-dioxa-2-thia-3,5-diazacyclononadeca[1,2,3-cd]indene-7-carboxylate

To a solution of Example 134H (85 mg) in N,N-dimethylformamide (4 mL) was added 1-methylpiperazine (255 mg). The mixture was stirred at 40° C. for three days. The mixture was diluted with ethyl acetate (100 mL), washed with water and brine, dried over Na₂SO₄, and filtered. Evaporation of the solvent provided the title compound. MS (ESI) m/z 929.5 (M+H)⁺.

### Example 134J

### (7R,16S,21S)-19-chloro-1-(4-fluorophenyl)-10-{[2-(2-methoxyphenyl)pyrimidin-4-yl]methoxy}-20-methyl-16-[(4-methylpiperazin-1-yl)methyl]-7,8,15,16-tetrahydro-14H-18,21-etheno-9,13-(metheno)-6,17-dioxa-2-thia-3,5-diazacyclononadeca[1,2,3-cd]indene-7-carboxylic acid

The title compound was prepared as described in Example 10F, substituting Example 134I for Example 10E. ¹H NMR (400 MHz, dimethyl sulfoxide-d₆) δ ppm 8.89 (d, 1H), 8.73 (s, 1H), 7.60-7.43 (m, 4H), 7.33-7.14 (m, 7H), 7.07 (t, 1H), 7.01 (d, 1H), 6.93 (dd, 1H), 6.87 (d, 1H), 5.92 (dd, 1H), 5.84 (d, 1H), 5.31-5.10 (m, 2H), 3.98 (dq, 2H), 3.78 (s, 3H), 2.76 (s, 3H), 2.43 (dd, 1H), 2.36 (s, 3H), 2.09 (q, 2H), 1.15 (d, 2H). MS (ESI) m/z 901.2 (M+H)⁺.

### Example 135

### (7S,16S,21S)-19-chloro-1-(4-fluorophenyl)-10-{[2-(2-methoxyphenyl)pyrimidin-4-yl]methoxy}-20-methyl-16-[(4-methylpiperazin-1-yl)methyl]-7,8,15,16-tetrahydro-14H-18,21-etheno-9,13-(metheno)-6,17-dioxa-2-thia-3,5-diazacyclononadeca[1,2,3-cd]indene-7-carboxylic acid

The title compound was isolated as a minor product from Example 134J. ¹H NMR (501 MHz, dimethyl sulfoxide-d₆) δ ppm 8.80 (d, 1H), 8.65 (s, 1H), 7.54-7.42 (m, 2H), 7.36 (d, 1H), 7.30-7.25 (m, 2H), 7.22-7.12 (m, 4H), 7.10-6.96 (m, 4H), 6.79 (d, 1H), 6.46 (d, 1H), 5.70 (d, 1H), 5.03 (s, 2H), 4.79 (s, 1H), 3.77 (d, 3H), 3.11 (dd, 1H), 2.79 (s, 3H), 2.72-2.55 (m, 1H), 2.43-2.34 (m, 3H), 2.07 (d, 1H), 1.97 (s, 3H). MS (ESI) m/z 901.5 (M+H)⁺.

### Example 136

### (7R,16R,21S)-10-(benzyloxy)-19-chloro-1-(4-fluorophenyl)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid

### Example 136A

### tert-butyl 2-acetoxy-2-(diethoxyphosphoryl)acetate

A 3 L jacketed round bottom flask equipped with an overhead stirrer was charged with glyoxylic acid monohydrate (15 g) and diethyl phosphite (20.82 mL) and was heated to a 60° C. jacket temperature with stirring. The flask headspace was continuously purged with a nitrogen sweep. After stirring overnight, dichloromethane (250 mL) was added, the reaction was cooled to an internal temperature of 5° C. and pyridine (13.05 mL) was added dropwise. After stirring for 1 hour at the same temperature, acetyl chloride (11.47 mL) was added dropwise over 20 minutes. The reaction was warmed to 20° C., stirred for 1.5 hours, and cooled to 5° C. internal temperature. Pyridine (19.57 mL) was added slowly. Tert-butanol (15.43 mL) was added in one portion followed by dropwise addition of 2,4,6-tripropyl-1,3,5,2,4,6-trioxatriphosphinane 2,4,6-trioxide (144 mL, 50% by weight in ethyl acetate) over 20 minutes. After stirring for 1 hour, the reaction was warmed to 20° C. and was stirred overnight. The reactor was then cooled to 5° C. and 1 N aqueous hydrochloric acid (200 mL) was added slowly. The biphasic mixture was stirred for 30 minutes at 20° C., and poured into a separatory funnel. Dichloromethane (400 mL) and 1N aqueous hydrochloric acid (250 mL) were added and the mixture was separated. The aqueous layer was extracted with dichloromethane (400 mL), and the combined organic layers were washed with a mixture of water (300 mL) and saturated aqueous sodium chloride solution (300 mL). The combined organics were dried over anhydrous magnesium sulfate, filtered and concentrated under reduced pressure. The crude material was purified by plug filtration on silica gel eluting with 1:1 ethyl acetate/heptanes to give the title compound after concentration under reduced pressure. ¹H NMR (400 MHz, Chloroform-d) δ ppm 5.32 (d, 1H), 4.29-4.18 (m, 4H), 2.21 (s, 3H), 1.37 (tdd, 6H). MS (ESI) m/z 255.0 (M-tert-butyl+2H)⁺.

### Example 136B

### (E)-tert-butyl 2-acetoxy-3-(2-(benzyloxy)-5-((tert-butyldimethylsilyl)oxy)phenyl)acrylate

An oven dried 2 L 3-neck round bottomed flask equipped with overhead stirring was charged with anhydrous lithium chloride (5.55 g). The flask was purged with a sweep of argon for 10 minutes and anhydrous tetrahydrofuran (350 mL) was added. A solution of Example 136A (40.6 g) in tetrahydrofuran (50 mL) was added. A solution of 1,8-diazabicyclo[5.4.0]undec-7-ene) (19.72 mL) in tetrahydrofuran (50 mL) was added dropwise. The stirring mixture became cloudy and was cooled in an ice-water bath to an internal temperature of 15° C. A mixture of Example 16A (32 g) in tetrahydrofuran (50 mL) was added over 30 minutes. The reaction was stirred overnight, cooled to an internal temperature of 5° C., and quenched by addition of 1% by weight aqueous citric acid (700 mL). Ethyl acetate (400 mL) was added and the layers were separated. The combined organic layers were washed with saturated aqueous sodium chloride solution (400 mL), dried over anhydrous magnesium sulfate, filtered and concentrated under reduced pressure. The crude material was purified by flash column chromatography on a Grace Reveleris system using a Teledyne Isco RediSep® Gold 330 g column, eluting with a 0-25% ethyl acetate/heptanes gradient to give the title compound in a 9:1 mixture of E- and Z-isomers. E-isomer ¹H NMR (501 MHz, Chloroform-d) δ ppm 7.39 (ddt, 2H), 7.36 (ddd, 2H), 7.32-7.27 (m, 1H), 6.88 (dd, 1H), 6.85 (d, 1H), 6.76 (d, 1H), 6.71 (ddd, 1H), 5.01 (s, 2H), 2.22 (s, 3H), 1.34 (s, 9H), 0.97 (s, 9H), 0.17 (s, 6H). MS (ESI) m/z 515.9 (M+NH₄)⁺. This isomer was assigned E by 2D NOE experiments. Z-isomer: ¹H NMR (501 MHz, Chloroform-d) δ ppm 7.74 (s, 1H), 7.45 (ddt, 2H), 7.38 (ddd, 2H), 7.35-7.30 (m, 1H), 7.29-7.26 (m, 1H), 6.83 (d, 1H), 6.79 (dd, 1H), 5.06 (s, 2H), 2.30 (d, 3H), 1.53 (s, 9H), 0.99 (s, 9H), 0.18 (s, 6H). MS (ESI) m/z 515.9 (M+NH₄)⁺. This isomer was assigned Z by 2D NMR experiments.

### Example 136C

### (R)-tert-butyl 2-acetoxy-3-(2-(benzyloxy)-5-((tert-butyldimethylsilyl)oxy)phenyl)propanoate

A 600 mL stainless steel reactor was charged with (1,2-bis[(2R,5R)-2,5-diethylphospholano]benzene(1,5-cyclooctadiene)rhodium(I) trifluoromethanesulfonate (1.88 g), followed by a solution of Example 136B (34.86 g) in methanol (350 mL). The reactor was purged with nitrogen 3 times and 2 times with hydrogen. The mixture was stirred at 1200 RPM under 120 psi of hydrogen with no external heating for 24 hours. The mixture was concentrated under reduced pressure, suspended in 5:1 heptanes/dichloromethane (70 mL) and filtered through a pad of diatomaceous earth. The filtrate was concentrated under reduced pressure and purified on a Grace Reveleris system using a 750 g Teledyne Isco Redisep gold column eluting with an ethyl acetate/heptanes gradient (0-25%). The title compound was concentrated under reduced pressure. ¹H NMR (400 MHz, Chloroform-d) δ ppm 7.45 (d, 2H), 7.42-7.34 (m, 2H), 7.34-7.28 (m, 1H), 6.77 (d, 1H), 6.70 (d, 1H), 6.67 (dd, 1H), 5.19 (dd, 1H), 5.05 (d, 1H), 5.01 (d, 1H), 3.29 (dd, 1H), 2.92 (dd, 1H), 2.03 (s, 3H), 1.40 (s, 9H), 0.97 (s, 9H), 0.16 (s, 6H). MS (DCI) m/z 518.2 (M+NH₄)⁺.

### Example 136D

### (R)-tert-butyl 3-(2-(benzyloxy)-5-((tert-butyldimethylsilyl)oxy)phenyl)-2-hydroxypropanoate

An oven dried 250 mL 3-neck flask was charged with Example 136C (27.46 g). The flask was equipped with a magnetic star bar and rubber septa, and vacuum purged with dinitrogen twice. Anhydrous ethanol (274 mL) was added, and the mixture stirred. To the stirring solution was added dropwise sodium ethoxide (21% wt in ethanol, 1.024 mL). The reaction was stirred for three hours at ambient temperature and quenched by addition of acetic acid (0.3 mL). The bulk of the solvents were removed by rotary evaporation, and the material was diluted with ethyl acetate (300 mL). Saturated aqueous sodium bicarbonate was added (300 mL). The layers were separated and the aqueous layer was extracted with ethyl acetate (300 mL). The combined organic layers were washed with saturated aqueous sodium chloride, dried over MgSO₄, treated with activated charcoal (0.5 g) and stirred for 1 hour before filtering through diatomaceous earth to give the title compound after concentration under reduced pressure. ¹H NMR (400 MHz, chloroform-d) δ ppm 7.48-7.42 (m, 2H), 7.42-7.36 (m, 2H), 7.36-7.29 (m, 1H), 6.79 (d, 1H), 6.75 (d, 1H), 6.67 (dd, 1H), 5.10-4.99 (m, 2fH), 4.39 (ddd, 1H), 3.16 (dd, 1H), 2.91 (d, 1H), 2.86 (dd, 1H), 1.41 (s, 9H), 0.99 (s, 9H), 0.18 (s, 6H). MS (DCI) m/z 476.2 (M+NH₄)⁺.

### Example 136E

### (R)-tert-butyl 3-(2-(benzyloxy)-5-((tert-butyldimethylsilyl)oxy)phenyl)-2-((5-bromo-6-(4-fluorophenyl)thieno[2,3-d]pyrimidin-4-yl)oxy)propanoate

A 1 L flask containing Example 136D (24.03 g) and Example 1D (19.08 g) was equipped with a stir bar, thermocouple for internal temperature monitoring and sealed with a rubber septum. The flask was flushed with argon, and warm tert-butanol (262 mL) was added via cannula. Cesium carbonate (51.2 g) was added in one portion. The reaction was heated to an internal temperature of 65° C. After four hours at this temperature, the reaction was allowed to cool to ambient temperature, diluted with methyl tert-butyl ether (100 mL) and filtered through a pad of diatomaceous earth. The filter pad was washed with ethyl acetate (2×100 mL). The solvents were evaporated, and the crude material was re-dissolved in ethyl acetate (500 mL). The mixture was washed with water (300 mL) and saturated sodium chloride solution (300 mL), dried over anhydrous magnesium sulfate, filtered, and concentrated. The crude residue was purified on a Grace Reveleris instrument using a Teledyne Isco Redisep Gold 750 g column, eluting with a 0-30% ethyl acetate/heptanes gradient. The desired fractions were combined and concentrated to give the title compound. ¹H NMR (501 MHz, Chloroform-d) δ 8.49 (s, 1H), 7.68-7.59 (m, 2H), 7.48-7.44 (m, 2H), 7.39-7.32 (m, 2H), 7.32-7.27 (m, 1H), 7.21-7.13 (m, 2H), 6.91 (d 1H), 6.77 (d, 1H), 6.65 (dd, 1H), 5.76 (dd, 1H), 5.07 (d, 1H), 5.04 (d, 1H), 3.49 (dd, 1H), 3.26 (dd, 1H), 1.40 (s, 9H), 0.93 (s, 9H), 0.11 (s, 3H), 0.10 (s, 3H). MS (ESI) m/z 765.2 (M+H)⁺.

### Example 136F

### (3-chloro-4-hydroxy-2-methylphenyl)boronic acid

A 5 L 3 neck jacketed flask equipped with overhead stirring and thermocouple for internal temperature monitoring was charged with Example 64C (50 g), chloro[(tri-tert-butylphosphine)-2-(2-aminobiphenyl)]palladium(II) (5.78 g), tetrahydroxydiboron (60.7 g), and potassium acetate (55.4 g) which had been dried overnight under vacuum at 50° C. The flask was flow purged with an N₂ sweep for 2 hours, and cooled until the internal temperature of the material reached −6° C. An oven dried 2 L round bottomed flask was charged with anhydrous methanol (1129 mL) and anhydrous ethylene glycol (376 mL). The stirring solvents were degassed by subsurface sparging with nitrogen gas for two hours and were cooled to −8° C. in an ice/ethanol bath. The solvent mixture was transferred to the reaction flask via cannula over 10 minutes. The reaction was stirred at −7° C. for 2.5 hours, quenched by addition of water (1 L), and allowed to stir at 0° C. for 1 hour. The mixture was filtered through a large pad of diatomaceous earth and the filter pad was washed with 1:1 water/methanol (2×500 mL). The filtrate was concentrated on a rotary evaporator until approximately 1.5 L of solvent had been removed. The mixture was extracted with ethyl acetate (2×1 L). The combined organic extracts were washed with brine, dried over anhydrous magnesium sulfate, filtered, and concentrated under reduced pressure. The crude material was treated with dichloromethane (200 mL), and the title compound was collected by filtration. ¹H NMR (400 MHz, dimethylsulfoxide-d₆/deuterium oxide) δ ppm 7.19 (d, 1H), 6.75 (d1H), 2.38 (s, 3H). MS (ESI) m/z 412.9 (M−H)⁻.

### Example 136G

### (R)-tert-butyl 3-(2-(benzyloxy)-5-((tert-butyldimethylsilyl)oxy)phenyl)-2-(((1S)-5-(3-chloro-4-hydroxy-2-methylphenyl)-6-(4-fluorophenyl)thieno[2,3-d]pyrimidin-4-yl)oxy)propanoate

A 1 L 3 neck flask equipped with overhead stirring was charged with Example 136E (30.2 g), 4-(di-tert-butylphosphino)-N,N-dimethylaniline (1.15 g), (tris(dibenzylideneacetone)dipalladium(0)) (1.806 g), and Example 136F (14.70 g). The flask was sealed with rubber septa and was flushed with argon for 15 minutes. A separate 500 mL round bottomed flask equipped with a magnetic stir bar was charged with cesium carbonate (25.7 g) and was sealed with a septum. The flask was flushed with argon for 10 minutes and water (46.9 mL) and 1,4-dioxane (235 mL) were added. The flask was degassed by subsurface sparging with stirring for 30 minutes and the contents were transferred to the reaction flask via cannula. The reaction was stirred for 60 hours and was quenched by addition of ammonium pyrrolidine-1-carbodithioate (1.296 g). The reaction was stirred for 1 hour at which point ethyl acetate (200 mL) and water (100 mL) were added. The biphasic mixture was filtered through a pad of diatomaceous earth, washing with ethyl acetate (100 mL) and water (50 mL). The layers were separated and the aqueous layer was extracted with ethyl acetate (200 mL). The combined organic layers were washed with a solution of saturated aqueous sodium chloride, dried over anhydrous magnesium sulfate, filtered and concentrated under reduced pressure. The crude material was purified by flash column chromatography using a Grace Reveleris system using a Teledyne Isco Redisep Gold 750 g column eluting with a 0-30% ethyl acetate/heptanes gradient. The pure fractions were collected and concentrated under reduced pressure to give the title compound. ¹H NMR (501 MHz, dimethylsulfoxide-d₆) δ ppm 10.10 (s, 1H), 8.61 (s, 1H), 7.43-7.38 (m, 2H), 7.36-7.24 (m, 5H), 7.24-7.18 (m, 2H), 6.92 (d, 1H), 6.89 (d, 1H), 6.80 (d, Hz, 1H), 6.68 (dd, 1H), 6.43 (d, 1H), 5.34 (t, 1H), 5.03 (s, 2H), 2.70-2.60 (m, 2H), 1.91 (s, 3H), 1.17 (s, 9H), 0.89 (s, 9H), 0.09 (s, 3H), 0.08 (s, 3H). MS (ESI) m/z 827.1 (M+H)⁺.

### Example 136H

### (S)-3-(allyloxy)-2-hydroxypropyl 4-methylbenzenesulfonate

A 1 L 3 necked round bottomed flask equipped with a magnetic stir bar was charged with a solution of Example 116J (45.8 g) in dichloromethane (500 mL). 4-Dimethylaminopyridine (0.572 g) and N-ethyl-N-isopropylpropan-2-amine (60.3 mL) were then added sequentially. Solid 4-methylbenzene-1-sulfonyl chloride (33 g) was added portionwise and the reaction was heated to an internal temperature of 40° C. overnight. Upon cooling to ambient temperature, a solution of saturated aqueous ammonium chloride was added (300 mL). The layers were separated, and the organic layer was washed with a solution of saturated sodium chloride (200 mL), dried over anhydrous magnesium sulfate, filtered and concentrated under reduced pressure. The crude material was purified by flash column chromatography on a Grace Reveleris System using a Teledyne Isco Redisep Gold 750 g column eluting with a 0-40% ethyl acetate/heptanes gradient to give the title compound. ¹H NMR (400 MHz, chloroform-d) δ ppm 7.79 (d, 2H), 7.35 (d, 2H), 5.82 (ddt, 1H), 5.22 (dq,), 5.16 (dq, 1H), 4.10 (dd, 1H), 4.04 (dd, 1H), 3.98 (dd, 1H), 3.94 (dt, 2H), 3.47 (dd, 1H), 3.43 (dd, 1H), 2.87 (d, 1H), 2.44 (s, 3H). MS (ESI) m/z 304.0 (M+NH₄)⁺.

### Example 136I

### (R)-tert-butyl 2-(((1S)-5-(4-(((R)-1-(allyloxy)-3-(tosyloxy)propan-2-yl)oxy)-3-chloro-2-methylphenyl)-6-(4-fluorophenyl)thieno[2,3-d]pyrimidin-4-yl)oxy)-3-(2-(benzyloxy)-5-((tert-butyldimethylsilyl)oxy)phenyl)propanoate

An oven dried 250 mL 3-necked flask was charged with Example 136H (3.11 g) and Example 136G (5.0 g). The flask was equipped with a magnetic stir bar, sealed with rubber septa, and purged with an argon sweep for 15 minutes. Toluene (30 mL) was added and upon dissolution, the flask was cooled in an ice bath to an internal temperature of 5° C. Triphenylphosphine (3.17 g) was added and the reaction mixture was stirred for 5 minutes at which point di-tert-butyl azodicarboxylate (2.78 g) was added. After 30 minutes, the cooling bath was removed and the flask was allowed to warm to ambient temperature and stirred overnight. The reaction mixture was loaded onto a 400 mL Buchner funnel packed with silica gel which had been equilibrated with heptanes. The silica gel plug was eluted with a mixture of 1:3 ethyl acetate/heptanes (600 mL), which was concentrated. The crude product was purified by flash column chromatography on a Teledyne Isco Combiflash Rf instrument using a Teledyne Isco RediSep® Gold 220 g column. The pure fractions were combined and concentrated to give the title compound. ¹H NMR (400 MHz, dimethylsulfoxide-d₆) δ ppm 8.62 (s, 1H), 7.75 (d, 1H), 7.46-7.33 (m, 5H), 7.33-7.25 (m, 3H), 7.22 (t, 2H), 7.09 (d, 1H), 6.96 (d, 1H), 6.91 (d, 1H), 6.67 (dd, 1H), 6.39 (d, 1H), 5.62 (ddt, 1H), 5.31 (dd, 1H), 5.06-4.99 (m, 3H), 4.97 (dq, 1H), 4.69 (dt, 1H), 4.28 (dd, 1H), 4.18 (dd, 1H), 3.73 (dq, 2H), 3.45 (d, 2H), 2.58 (qd, 2H), 2.38 (s, 3H), 1.94 (s, 3H), 1.15 (s, 9H), 0.88 (s, 9H), 0.08 (s, 3H), 0.08 (s, 3H). MS (ESI) m/z 1095.3 (M+H)⁺.

### Example 136J

### (R)-tert-butyl 2-(((1S)-5-(4-(((R)-1-(allyloxy)-3-(tosyloxy)propan-2-yl)oxy)-3-chloro-2-methylphenyl)-6-(4-fluorophenyl)thieno[2,3-d]pyrimidin-4-yl)oxy)-3-(2-(benzyloxy)-5-hydroxyphenyl)propanoate

A 100 mL round bottomed flask was charged with Example 136I (3.58 g), sealed with a septum and purged with nitrogen gas for 10 minutes. Tetrahydrofuran (23 mL) was added followed by acetic acid (0.3 mL). The stirring homogeneous solution was cooled in an ice bath to 5° C. internal temperature and a solution of tetra-N-butylammonium fluoride (4.75 mL, 1M) in tetrahydrofuran was added dropwise. After 1 hour, the reaction was quenched by addition of saturated aqueous sodium bicarbonate (40 mL), and diluted with methyl tert-butyl ether (160 mL). The layers were separated and the organic layer was washed sequentially with water and brine, then dried over MgSO₄, filtered and concentrated. The crude residue was purified by flash column chromatography on a Teledyne Isco Combiflash Rf instrument using a Teledyne Isco RediSep® Gold 80 g column eluting with a 0-60% ethyl acetate/heptanes gradient. The desired fractions were collected, combined and concentrated to give the title compound. ¹H NMR (400 MHz, dimethylsulfoxide-d₆) δ ppm 8.78 (s, 1H), 8.61 (s, 1H), 7.80-7.70 (m, 2H), 7.45-7.40 (m, 2H), 7.40-7.33 (m, 4H), 7.32-7.24 (m, 3H), 7.24-7.19 (m, 2H), 7.13 (d, 1H), 7.01 (d, 1H), 6.83 (d, 1H), 6.57 (dd, 1H), 6.17 (d, 1H), 5.63 (ddt, 1H), 5.21 (dd, 1H), 5.04 (dq, 1H), 4.98 (ddt, 3H), 4.73 (dt, 1H), 4.29 (dd, 1H), 4.19 (dd, Hz, 1H), 3.75 (q, 1H), 3.74 (q, 1H), 3.48 (d, 2H), 2.59 (dd, 1H), 2.50 (d, 1H), 2.38 (s, 3H), 1.93 (s, 3H), 1.17 (s, 9H). MS (ESI) m/z 981.1 (M+H)⁺.

### Example 136K

### tert-butyl (7R,16R,21S)-10-(benzyloxy)-19-chloro-1-(4-fluorophenyl)-16-(allyloxymethyl)-20-methyl-7,8,15,16-tetrahydro-18,21-etheno-13,9-(metheno)-6,14,17-trioxa-2-thia-3,5-diazacyclononadeca[1,2,3-cd]indene-7-carboxylate

An oven dried 3 neck 500 mL round bottomed flask was charged with Example 136J (3.13 g), and equipped with a magnetic stir bar and sealed with rubber septa. The flask was purged with an argon flow for 10 minutes. N,N-Dimethylformamide (319 mL) was added and the material dissolved with stirring at ambient temperature. Cesium carbonate (5.19 g) was added and the suspension was stirred at ambient temperature for 3 hours. Ethyl acetate (100 mL) was added and the mixture was filtered through a pad of diatomaceous earth. The solvents were concentrated under vacuum, and the crude residue was treated with ethyl acetate (200 mL) and water (100 mL). A 1 M aqueous solution of lithium chloride was added (50 mL), and the layers were separated. The organic layer was dried over anhydrous magnesium sulfate, filtered and concentrated under reduced pressure. The crude residue was purified by flash column chromatography on a Teledyne Isco Combiflash Rf instrument using a Teledyne Isco RediSep® Gold 120 g column eluting with a 0-50% ethyl acetate/heptanes gradient. The desired fractions were collected, combined and concentrated to give the title compound. ¹H NMR (400 MHz, dimethylsulfoxide-d₆) δ ppm 8.70 (s, 1H), 7.49-7.43 (m, 3H), 7.43-7.36 (m, 3H), 7.37-7.29 (m, 1H), 7.26-7.14 (m, 6H), 6.97-6.91 (m, 3H), 6.88 (dd, 1H), 5.97 (dd, 1H), 5.89 (ddt, 1H), 5.52 (d, 1H), 5.27 (dq, 1H), 5.16 (dq, 1H), 5.04 (d, 1H), 4.97 (d, 1H), 4.50 (hept, 1H), 4.46-4.41 (m, 1H), 4.41-4.37 (m, 1H), 4.06-3.97 (m, 1H), 4.01-3.92 (m, 1H), 3.76 (dd, 1H), 3.68 (dd, 1H), 3.62 (dd, 1H), 2.71 (d, 1H), 2.23 (s, 3H), 1.01 (s, 9H). MS (ESI) m/z 809.1 (M+H)⁺.

### Example 136L

### tert-butyl (7R,16R,21S)-10-(benzyloxy)-19-chloro-1-(4-fluorophenyl)-16-(hydroxymethyl)-20-methyl-7,8,15,16-tetrahydro-18,21-etheno-13,9-(metheno)-6,14,17-trioxa-2-thia-3,5-diazacyclononadeca[1,2,3-cd]indene-7-carboxylate

An oven dried 100 mL round bottomed flask was charged with Example 136K (2.23 g), tetrakis(triphenylphosphine)palladium(0) (0.318 g), 1,3-dimethylpyrimidine-2,4,6(1H,3H,5H)-trione (0.946 g), and a magnetic stir bar, and sealed with a septum. The flask was purged with a flow of argon for 15 minutes. A mixture of tetrahydrofuran (18 mL) and methanol (9 mL) which was degassed by subsurface sparging with argon for 30 minutes was added via cannula. The reaction was stirred at ambient temperature for 40 hours at which point ammonium pyrrolidine-1-carbodithioate (0.181 g) was added and the stirring was continued for 1 hour. The reaction mixture was filtered through a plug of diatomaceous earth, and the filter pad was washed with ethyl acetate (25 mL) and water (25 mL). The filtrate layers were separated and the aqueous layer was extracted once with ethyl acetate (25 mL). The combined organic layers were washed with a solution of saturated aqueous sodium chloride (50 mL), dried over anhydrous magnesium sulfate and concentrated under reduced pressure. The crude residue was purified by flash column chromatography on a Teledyne Isco Combiflash Rf instrument using a Teledyne Isco RediSep® Gold 80 g column eluting with a 0-50% ethyl acetate/heptanes gradient. The pure fractions were collected, combined and concentrated to give the title compound. ¹H NMR (400 MHz, dimethylsulfoxide-d₆) δ ppm 8.70 (s, 1H), 7.50-7.43 (m, 2H), 7.44-7.36 (m, 2H), 7.37-7.30 (m, 1H), 7.26-7.14 (m, 5H), 6.98-6.90 (m, 2H), 6.86 (dd, 1H), 5.96 (dd, 1H), 5.52 (d, 1H), 5.04 (d, 1H), 4.98 (q, 2H), 4.48-4.31 (m, 3H), 3.76 (dd, 1H), 3.69 (ddd, 1H), 3.56 (dt, 1H), 2.77-2.66 (m, 1H), 2.23 (s, 3H), 1.02 (s, 9H). MS (ESI) m/z 769.2 (M+H)⁺.

### Example 136M

### tert-butyl (7R,16R,21S)-10-(benzyloxy)-19-chloro-1-(4-fluorophenyl)-20-methyl-16-{[(4-methylbenzene-1-sulfonyl)oxy]methyl}-7,8,15,16-tetrahydro-18,21-etheno-13,9-(metheno)-6,14,17-trioxa-2-thia-3,5-diazacyclononadeca[1,2,3-cd]indene-7-carboxylate

A 50 mL round bottomed flask was charged with Example 136L (1.81 g), and a magnetic stir bar. Dichloromethane was added (16 mL), and the mixture was stirred to dissolution. 1,4-Diazabicyclo[2.2.2]octane (0.660 g) and p-toluenesulfonyl chloride (0.673 g) were added sequentially. The reaction was stirred at ambient temperature for 1 hour and quenched by addition of ethylenediamine (0.079 mL). The reaction mixture was stirred for 10 minutes and was diluted with dichloromethane (20 mL). A solution of 1.0 M sodium dihydrogen phosphate NaH₂PO₄ (30 mL) was added. The layers were separated and the aqueous layer was extracted with dichloromethane (20 mL). The combined organic layers were dried over anhydrous magnesium sulfate, filtered and concentrated to give the title compound which was used without further purification. ¹H NMR (400 MHz, dimethylsulfoxide-d₆) δ ppm 8.70 (s, 1H), 7.84-7.77 (m, 2H), 7.46 (ddd, 4H), 7.44-7.37 (m, 2H), 7.37-7.31 (m, 1H), 7.20 (d, 3H), 7.11-7.04 (m, 1H), 6.94 (d, 1H), 6.92 (d, 1H), 6.87 (dd, 1H), 5.97 (dd, 1H), 5.48 (d, 1H), 5.06 (d, 1H), 4.99 (d, 1H), 4.61-4.49 (m, 1H), 4.39-4.32 (m, 3H), 4.29 (dd, 1H), 3.75 (dd, 1H), 2.75-2.64 (m, 1H), 2.40 (s, 3H), 2.21 (s, 3H), 1.01 (s, 9H). MS (ESI) m/z 923.0 (M+H)⁺.

### Example 136N

### tert-butyl (7R,16R,21S)-10-(benzyloxy)-19-chloro-1-(4-fluorophenyl)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

An oven dried 100 mL round bottomed flask was charged with Example 136M (2.17 g) and a magnetic stir bar then sealed with a rubber septum. The flask was purged with a nitrogen gas sweep for 10 minutes. Dimethylformamide (8 mL) and 1-methylpiperazine (8 mL) were added sequentially. The reaction was stirred for 60 hours at ambient temperature and 16 hours at 30° C. The reaction was cooled in an ice bath, and diluted with ethyl acetate (20 mL) and water (20 mL). The reaction was allowed to warm to ambient temperature and further diluted with water (80 mL) and ethyl acetate (80 mL). The layers were separated and the aqueous layer was extracted with ethyl acetate (2×50 mL). The combined organic layers were washed sequentially with water and a 0.5 M aqueous solution of lithium chloride, dried over anhydrous magnesium sulfate, and concentrated. The crude residue was purified by flash column chromatography on a Teledyne Isco Combiflash Rf instrument using a Teledyne Isco RediSep® Gold 80 g column eluting with a 0-10% methanol/dichlormethane gradient to yield the title compound. ¹H NMR (501 MHz, dimethylsulfoxide-d₆) δ ppm 8.71 (s, 1H), 7.47-7.43 (m, 3H), 7.43-7.37 (m, 3H), 7.37-7.29 (m, 2H), 7.26-7.13 (m, 5H), 6.93 (d, J=2.9 Hz, 1H), 6.91 (d, J=3.7 Hz, 1H), 6.82 (dd, J=9.0, 2.9 Hz, 2H), 6.01 (dd, J=5.9, 2.3 Hz, 2H), 5.53 (d, J=2.7 Hz, 1H), 5.06 (d, J=12.1 Hz, 1H), 4.98 (d, J=12.1 Hz, 1H), 4.48 (d, J=13.2 Hz, 1H), 4.44 (dd, J=8.2, 5.5 Hz, 1H), 4.32 (dd, J=13.0, 8.4 Hz, 1H), 3.78 (dd, J=16.7, 5.9 Hz, 1H), 2.75-2.68 (m, 1H), 2.60-2.55 (m, 1H), 2.54 (dd, J=13.0, 7.8 Hz, 1H), 2.31 (d, J=29.0 Hz, 8H), 2.24 (s, 3H), 2.15 (s, 3H), 1.01 (s, 9H). MS (ESI) m/z 851.0 (M+H)⁺.

### Example 136O

### (7R,16R,21S)-10-(benzyloxy)-19-chloro-1-(4-fluorophenyl)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid

A 1 dram vial was charged with Example 136N (25 mg) and was equipped with a magnetic stir bar and septum screw cap. Dichloromethane (0.2 mL) and trifluoroacetic acid (0.2 mL) were sequentially added and the reaction mixture was stirred for 5 hours. The volatiles were evaporated under a stream of nitrogen and the residue was purified by preparative reversed phase high pressure liquid chromatography on a Gilson PLC 2250 system equipped with a Phenomenex® Luna™ C18(2) 50×250 mm column eluting with a 10-90% acetonitrile/(0.1% aqueous trifluoroacetic acid) gradient. The volatiles were removed by lyophilization to give the title compound as the bis-trifluoroacetic acid salt. ¹H NMR (501 MHz, dimethylsulfoxide-d₆) δ 9.50 (s, 1H), 8.73 (s, 1H), 7.44 (d, 2H), 7.39 (dd, 2H), 7.36-7.29 (m, 1H), 7.22-7.16 (m, 4H), 7.14 (d, 1H), 6.95 (d, 1H), 6.89 (d, 1H), 6.81 (dd, 1H), 6.11 (dd, 1H), 5.65 (d, 1H), 5.07 (d, 1H), 5.00 (d, 1H), 4.57 (d, 1H), 4.48 (d, 1H), 4.35 (dd, 1H), 3.77 (dd, 1H), 3.12-2.96 (m, 4H), 2.91-2.81 (m, 1H), 2.80 (s, 3H), 2.74-2.61 (m, 2H), 2.20 (s, 3H). MS (ESI) m/z 795.4 (M+H)⁺.

### Example 137

### (7S,16R)-19,23-dichloro-1-(4-fluorophenyl)-10-{[2-(2-methoxyphenyl)pyrimidin-4-yl]methoxy}-16-[(4-methylpiperazin-1-yl)methyl]-7,8,15,16-tetrahydro-18,21-etheno-9,13-(metheno)-2,6,14,17-tetraoxa-3,5-diazacyclononadeca[1,2,3-cd]indene-7-carboxylic acid

The title compound was isolated as a minor product during the synthesis of Example 129H. ¹H NMR (500 MHz, dimethyl sulfoxide-d₆,) δ ppm 13.08 (s, 1H), 9.36 (s, 1H), 8.90 (d, 1H), 8.65 (s, 1H), 7.60 (d, 1H), 7.58 (d, 1H), 7.55-7.53 (m, 2H), 7.52-7.44 (m, 3H), 7.34-7.29 (m, 2H), 7.16 (d, 1H), 7.05 (t, 1H), 6.93 (d, 1H), 6.71 (dd, 1H), 6.35 (d, 1H), 6.32 (m, 1H), 5.18 (d, 2H), 5.14 (m, 1H), 4.33 (d, 1H), 4.14 (dd, 1H), 3.77 (s, 3H), 3.69 (br d, 1H), 3.66 (broad d, 1H), 3.29-3.14 (br m, 5H), 3.12-3.0 (br m, 3H), 2.97-2.84 (m, 2H), 2.81 (s, 3H). MS (ESI) m/z 907.2 (M+H)⁺.

### Example 138

### (7R,16R)-19-chloro-1-cyclobutyl-10-{[2-(2-methoxyphenyl)pyrimidin-4-yl]methoxy}-20-methyl-16-[(4-methylpiperazin-1-yl)methyl]-7,8,15,16-tetrahydro-18,21-etheno-13,9-(metheno)-2,6,14,17-tetraoxa-3,5-diazacyclononadeca[1,2,3-cd]indene-7-carboxylic acid

### Example 138A

### 5-bromo-4-chlorofuro[2,3-d]pyrimidine

4-Chlorofuro[2,3-d]pyrimidine (4 g) was dissolved in chloroform (15 mL). Acetic acid (1.63 mL) was added followed by bromine (4.00 mL). The reaction mixture was stirred for 16 hours at 25° C. The reaction mixture was diluted with additional chloroform (35 mL) and was cooled to 5° C. 1,8-Diazabicyclo[5.4.0]undec-7-ene (12 mL) was added. The reaction mixture was allowed to warm up to 25° C. and was stirred for a further 30 minutes. The reaction mixture was cooled to 5° C. and water (100 mL) was added. The mixture was extracted with dichloromethane (2×200 mL). The combined organic layers were washed with water and aqueous sodium thiosulfate solution, dried over MgSO₄, filtered, and concentrated in vacuo. The residue obtained was purified by silica gel flash chromatography (80 g Chromabond® column, gradient ethyl acetate in heptane 0-30%). The residue was dissolved in dichloromethane (20 mL), and pentane (80 mL) was added. The precipitated material was filtered off, washed with pentane and dried to give the title compound. MS (ESI) m/z 232.9/234.9 (M+H)⁺.

### Example 138B

### 4-chloro-5-(3-chloro-2-methyl-4-((triisopropylsilyl)oxy)phenyl)furo[2,3-d]pyrimidine

A mixture of Example 138A (740 mg), Example 134D (1500 mg), bis(di-tert-butyl(4-dimethylaminophenyl)phosphine)dichloropalladium(II) (200 mg) and tribasic potassium phosphate (1817 mg) were stirred under a nitrogen atmosphere. A solution of tetrahydrofuran (16 mL) and water (4 mL) was degassed and added. The mixture was stirred for 20 hours at room temperature, and additional Example 138B (500 mg) was added. After stirring for a further 3 hours at room temperature, the tetrahydrofuran was removed by rotary evaporation, water was added to the residue, and the mixture was extracted with ethyl acetate. The combined organic layers were washed with brine, dried over MgSO₄, filtered, and concentrated in vacuo. The residue obtained was purified by silica gel flash chromatography (25 g Chromabond® column, gradient ethyl acetate in heptane 0-30%) to give the title compound. MS (ESI) m/z 451.2 (M+H)⁺.

### Example 138C

### 6-bromo-4-chloro-5-(3-chloro-2-methyl-4-((triisopropylsilyl)oxy)phenyl)furo[2,3-d]pyrimidine

Example 138B (1.28 g) was dissolved in dimethylformamide (15 mL). N-Bromosuccinimide (800 mg) was added and the mixture was stirred for 3 hours at room temperature. Additional N-bromosuccinimide (500 mg) was added and stirring was continued for 21 hours. Additional N-bromosuccinimide (800 mg) was added and the reaction was stirred a further 8 hours. Additional N-bromosuccinimide (500 mg) was added and the reaction was stirred a further 16 hours. Additional N-bromosuccinimide (500 mg) was added and the reaction was stirred a further 8 hours. Additional N-bromosuccinimide (500 mg) was added and the reaction was stirred a further 16 hours. Water (100 mL) was added and the mixture was extracted with ethyl acetate. The combined organic layers were washed with 1 M aqueous hydrochloric acid solution and brine, dried over MgSO₄, filtered, and concentrated in vacuo. The residue was purified by silica gel flash chromatography (40 g Chromabond® column, gradient ethyl acetate in heptane 0-25%) to give the title compound. MS (ESI) m/z 531.1 (M+H)⁺.

### Example 138D

### (2R)-ethyl 2-((6-bromo-5-(3-chloro-2-methyl-4-((triisopropylsilyl)oxy)phenyl)furo[2,3-d]pyrimidin-4-yl)oxy)-3-(5-((tert-butyldimethylsilyl)oxy)-2-((2-(2-methoxyphenyl)pyrimidin-4-yl)methoxy)phenyl)propanoate

A mixture of Example 138C (210 mg), Example 68B (213 mg) and cesium carbonate (387 mg) in anhydrous tert-butanol (6 mL) was stirred for 5 hours at 70° C. Water was added and the mixture was extracted with ethyl acetate. The combined organic layers were washed with brine, dried over MgSO₄, filtered, and concentrated in vacuo. The residue was purified by silica gel flash chromatography (15 g Chromabond® column, gradient ethyl acetate in heptane 0-50%) to give the title compound. MS (ESI) m/z 1033.4 (M+H)⁺.

### Example 138E

### (2R)-ethyl 2-((6-bromo-5-(3-chloro-4-hydroxy-2-methylphenyl)furo[2,3-d]pyrimidin-4-yl)oxy)-3-(5-((tert-butyldimethylsilyl)oxy)-2-((2-(2-methoxyphenyl)pyrimidin-4-yl)methoxy)phenyl)propanoate

Example 138D (310 mg) was stirred in dimethylformamide (5 mL). A solution of potassium acetate (3 mg) in water (0.263 mL) was added. The reaction mixture was stirred for 5 hours at 25° C. Water (30 mL) and aqueous NaHCO₃ solution (1 M, 10 mL) were added and the mixture was extracted with ethyl acetate. The combined organic layers were washed with brine, dried over MgSO₄, filtered and concentrated in vacuo. The residue obtained was purified by silica gel flash chromatography (4 g Chromabond® column, gradient ethyl acetate in heptane 0-60%) to give the title compound. MS (ESI) m/z 877.2 (M+H)⁺.

### Example 138F

### (2R)-ethyl 2-((5-(4-(((R)-1-(bis(4-methoxyphenyl)(phenyl)methoxy)-3-(tosyloxy)propan-2-yl)oxy)-3-chloro-2-methylphenyl)-6-bromofuro[2,3-d]pyrimidin-4-yl)oxy)-3-(5-((tert-butyldimethylsilyl)oxy)-2-((2-(2-methoxyphenyl)pyrimidin-4-yl)methoxy)phenyl)propanoate

Example 138E (100 mg), Example 112B (75 mg), di-tert-butyl azodicarboxylate (39.4 mg) and triphenylphosphine (44.9 mg) were stirred together under argon in an ice-water cooling bath. Tetrahydrofuran (5 mL), followed by triethylamine (0.032 mL), were added. The mixture was stirred for 20 minutes in the cooling bath and at 25° C. for 2 days. Water was added and the mixture was extracted with ethyl acetate. The combined organic layers were washed with water, dried over MgSO₄, filtered and concentrated in vacuo. The residue was purified by silica gel flash chromatography (12 g Reveleris column, gradient ethyl acetate in heptane 1-60%) to give the title compound. MS (ESI) m/z 1407.4 (M+H)⁺.

### Example 138G

### (2R)-ethyl 2-((5-(4-(((R)-1-(bis(4-methoxyphenyl)(phenyl)methoxy)-3-(tosyloxy)propan-2-yl)oxy)-3-chloro-2-methylphenyl)-6-bromofuro[2,3-d]pyrimidin-4-yl)oxy)-3-(5-hydroxy-2-((2-(2-methoxyphenyl)pyrimidin-4-yl)methoxy)phenyl)propanoate

TBAF (tetrabutyl ammonium fluoride, 0.10 mL, 1 M solution in tetrahydrofuran) was added to a stirred, ice-water cooled solution of Example 138F (70 mg) in tetrahydrofuran (5 mL). After stirring for 25 minutes at 0-5° C., aqueous ammonium chloride solution (3 mL, 10%) was added and the mixture was extracted with ethyl acetate. The combined extracts were washed with water, dried over MgSO₄, and filtered. The solvent was reduced in vacuo. The residue was purified by silica gel flash chromatography (4 g Reveleris column, gradient ethyl acetate in heptane 1-75%) to give the title compound. MS (ESI) m/z 1293.4 (M+H)⁺.

### Example 138H

### ethyl (7R,16S)-16-{[bis(4-methoxyphenyl)(phenyl)methoxy]methyl}-1-bromo-19-chloro-10-{[2-(2-methoxyphenyl)pyrimidin-4-yl]methoxy}-20-methyl-7,8,15,16-tetrahydro-18,21-etheno-13,9-(metheno)-2,6,14,17-tetraoxa-3,5-diazacyclononadeca[1,2,3-cd]indene-7-carboxylate

To Example 138G (75 mg) dissolved in tetrahydrofuran (5 mL) was added Cs₂CO₃ (25 mg) and the reaction mixture was stirred for 24 hours at 50° C. To the reaction mixture was added water (40 mL) and the aqueous phase was extracted twice with ethyl acetate (20 mL). The combined organic extracts were washed twice with brine (20 mL), dried over MgSO₄, filtered, and concentrated in vacuo. The residue obtained was purified by silica gel flash chromatography (4 g Chromabond® column, gradient ethyl acetate in n-heptane 10-60%) to give the title compound. MS (ESI) m/z 1121.4 (M+H)⁺.

### Example 138I

### ethyl (7R,16R)-1-bromo-19-chloro-16-(hydroxymethyl)-10-{[2-(2-methoxyphenyl)pyrimidin-4-yl]methoxy}-20-methyl-7,8,15,16-tetrahydro-18,21-etheno-13,9-(metheno)-2,6,14,17-tetraoxa-3,5-diazacyclononadeca[1,2,3-cd]indene-7-carboxylate

To Example 138H (24 mg) dissolved in methanol (1 mL) and dichloromethane (1 mL) was added formic acid (0.5 mL) and the reaction mixture was stirred for 30 minutes at room temperature. To the reaction mixture was added water (30 mL) and the aqueous phase was extracted twice with dichloromethane (15 mL). The combined organic extracts were washed with water (20 mL) and saturated aqueous NaHCO₃ solution (20 mL), dried over MgSO₄, filtered, and concentrated in vacuo. The residue obtained was purified by silica gel flash chromatography (4 g Chromabond® column, gradient ethyl acetate in n-heptane 0-10%) to give the title compound. MS (ESI) m/z 819.0 (M+H)⁺.

### Example 138J

### ethyl (7R,16S)-1-bromo-19-chloro-10-{[2-(2-methoxyphenyl)pyrimidin-4-yl]methoxy}-20-methyl-16-{[(4-methylbenzene-1-sulfonyl)oxy]methyl}-7,8,15,16-tetrahydro-18,21-etheno-13,9-(metheno)-2,6,14,17-tetraoxa-3,5-diazacyclononadeca[1,2,3-cd]indene-7-carboxylate

To Example 138I (14 mg) dissolved in dichloromethane (2 mL) was added triethylamine (10 μL) and p-toluenesulfonyl chloride (7 mg). The reaction mixture was stirred for 16 hours at room temperature. Because the reaction was not complete, triethylamine (10 μL) and p-toluenesulfonyl chloride (7 mg) were added and the reaction mixture was stirred at reflux for 1 hour and subsequently at room temperature for 24 hours. To the reaction mixture was added water (30 mL) and saturated aqueous NaHCO₃ solution (10 mL). The aqueous phase was extracted twice with ethyl acetate. The organic phase was washed with brine, dried over MgSO₄, filtered, and concentrated in vacuo to give the title compound. MS (ESI) m/z 973.0 (M+H)⁺.

### Example 138K

### ethyl (7R,16R)-1-bromo-19-chloro-10-{[2-(2-methoxyphenyl)pyrimidin-4-yl]methoxy}-20-methyl-16-[(4-methylpiperazin-1-yl)methyl]-7,8,15,16-tetrahydro-18,21-etheno-13,9-(metheno)-2,6,14,17-tetraoxa-3,5-diazacyclononadeca[1,2,3-cd]indene-7-carboxylate

To Example 138J (19 mg) dissolved in N,N-dimethylformamide (4 mL) was added 1-methylpiperazine (72 mg). The reaction mixture was stirred at 55° C. for 48 hours. To the reaction mixture was added water (30 mL) and saturated aqueous NaHCO₃ solution (10 mL). The aqueous phase was extracted twice with ethyl acetate. The organic phase was washed with brine, dried over MgSO₄, filtered, and concentrated in vacuo. The residue obtained was purified by silica gel flash chromatography (4 g Chromabond® column, gradient methanol in dichloromethane 0-10%) to give the title compound. MS (ESI) m/z 901.2 (M+H)⁺.

### Example 138L

### ethyl (7R,16R)-19-chloro-1-cyclobutyl-10-{[2-(2-methoxyphenyl)pyrimidin-4-yl]methoxy}-20-methyl-16-[(4-methylpiperazin-1-yl)methyl]-7,8,15,16-tetrahydro-18,21-etheno-13,9-(metheno)-2,6,14,17-tetraoxa-3,5-diazacyclononadeca[1,2,3-cd]indene-7-carboxylate

To a dry 5 mL microwave vial, which was dried for 24 hours at 70° C. under vacuum and stored in a glove box, was added Example 138K (6 mg), potassium cyclobutyltrifluoroborate (3 mg), Cs₂CO₃ (5 mg), dichloro(4,4′-di-tert-butyl-2,2′-bipyridine)nickel (0.4 mg), and (4,4′-di-t-butyl-2,2′-bipyridine)bis[3,5-difluoro-2-[5-trifluoromethyl-2-pyridinyl-kN)phenyl-kC]iridium(III) hexafluorophosphate (1 mg) in a glove box. Dry dioxane (1.0 mL degassed with nitrogen) was added and the reaction mixture was exposed to blue light (40 W Kessil blue LEDs; vial was placed 4 cm in front of the light source). The reaction mixture was stirred at room temperature for 2 hours. The reaction mixture was diluted with water (20 mL) and extracted twice with ethyl acetate. The combined organic layers were washed with brine, dried over MgSO₄, filtered and concentrated in vacuo. The residue obtained was used without any further purification in the next step. MS (ESI) m/z 875.4 (M+H)⁺.

### Example 138M

### (7R,16R)-19-chloro-1-cyclobutyl-10-{[2-(2-methoxyphenyl)pyrimidin-4-yl]methoxy}-20-methyl-16-[(4-methylpiperazin-1-yl)methyl]-7,8,15,16-tetrahydro-18,21-etheno-13,9-(metheno)-2,6,14,17-tetraoxa-3,5-diazacyclononadeca[1,2,3-cd]indene-7-carboxylic acid

Example 138L (8 mg) was dissolved in ethanol (0.5 mL) and tetrahydrofuran (0.5 mL). LiOH (3.0 mg) was dissolved in water (0.5 mL) and was added to the reaction mixture. The reaction mixture was stirred overnight at room temperature. Because the reaction was not complete, additional LiOH (3.0 mg) was added and the reaction mixture was stirred for 72 hours at room temperature. Trifluoroacetic acid (26 μL) was added to the reaction mixture and the solvent was removed in vacuo. Purification by HPLC (Waters X-Bridge C18 19×150 mm, 5 μm column, gradient 5-95% acetonitrile+0.1% trifluoroacetic acid in water+0.1% trifluoroacetic acid) provided the title compound. ¹H NMR (400 MHz, dimethyl sulfoxide-d₆) δ ppm 13.23 (s, 1H), 9.34 (bs, 1H), 8.84 (d, 1H), 8.45 (s, 1H), 7.56 (d, 1H), 7.50 (d, 1H), 7.45 (m, 1H), 7.19 (d, 1H), 7.13 (d, 1H), 7.03 (m, 1H), 6.88 (m, 1H), 6.83 (m, 1H), 6.75 (m, 1H), 6.10 (s, 1H), 5.54 (m, 1H), 5.16-5.09 (m, 3H), 4.22 (m, 1H), 4.12 (m, 1H), 3.74 (s, 3H), 3.53 (m, 1H), 3.42 (m, 3H), 3.29 (m, 1H), 3.21 (m, 1H), 3.09 (m, 4H), 2.90 (m, 2H), 2.81 (m, 3H), 2.73 (m, 1H), 2.40-2.30 (m, 6H), 2.10 (m, 1H), 1.92 (m, 2H). MS (ESI) m/z 847.4 (M+H)⁺.

### Example 139

### (7R,16R,21S)-19-chloro-10-({2-[2-(difluoromethoxy)phenyl]pyrimidin-4-yl}methoxy)-1-(4-fluorophenyl)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid

### Example 139A

### 2-(4-(dimethoxymethyl)pyrimidin-2-yl)phenol

To a solution of 2-hydroxybenzene-1-carboximidamide hydrochloride (5 g) in ethanol (120 mL) was added sodium ethoxide (18.77 g) followed by Example 100A (5.52 mL) and the mixture was stirred at 70° C. overnight. After cooling to ambient temperature, the mixture was concentrated and the residue was treated with 100 mL of a 1:1 ethyl acetate:heptane mixture, and poured into a separatory funnel. The aqueous mixture was washed with one portion of saturated aqueous ammonium chloride, water, and saturated aqueous brine, then dried over anhydrous magnesium sulfate, filtered and concentrated. The crude material was carried through the next step without further purification. LC/MS (APCI) m/z 247.3 (M+H)⁺.

### Example 139B

### 2-(2-(difluoromethoxy)phenyl)-4-(dimethoxymethyl)pyrimidine

To a stirring mixture of Example 139A (6.5 g) in 130 mL of acetonitrile was added 130 mL of water. To the resulting slurry was added potassium hydroxide (29.6 g). After dissolution of the material, the mixture was cooled to −15° C. Next, diethyl (bromodifluoromethyl)phosphonate (10.57 g) was added in one portion. The mixture was stirred at −15° C. for one hour and the cooling bath was removed and the mixture was stirred at ambient temperature for 2 hours. The reaction mixture was poured into a separatory funnel, diluted with water, and extracted with diethyl ether. The organic layer was washed with saturated aqueous brine, dried over anhydrous magnesium sulfate, filtered and concentrated onto silica gel. Purification by flash chromatography on a CombiFlash® Teledyne Isco system using a Teledyne Isco RediSep® Rf gold 220 g silica gel column (eluting with 0-50% ethyl acetate in heptanes) afforded the title compound. LC/MS (APCI) m/z 297.3 (M+H)⁺.

### Example 139C

### 2-(2-(difluoromethoxy)phenyl)pyrimidine-4-carbaldehyde

To a stirring mixture of Example 139B (2.69 g) in tetrahydrofuran (56.7 mL) was added aqueous 1 M HCl (54.5 mL) and the mixture was stirred at 55° C. for 5 hours. After cooling, the reaction mixture was poured into a separatory funnel containing saturated aqueous sodium bicarbonate. The mixture was extracted with one portion of ethyl acetate, and the organic layer was washed with saturated aqueous brine, dried over anhydrous magnesium sulfate, filtered and concentrated to obtain the crude title compound. ¹H NMR (501 MHz, dimethyl sulfoxide-d₆) δ ppm 10.00 (d, J=0.7 Hz, 1H), 9.25 (dd, J=4.9, 0.7 Hz, 1H), 7.96 (dd, J=7.8, 1.8 Hz, 1H), 7.87 (d, J=5.0 Hz, 1H), 7.63 (ddd, J=8.2, 7.4, 1.8 Hz, 1H), 7.48 (td, J=7.6, 1.1 Hz, 1H), 7.41-7.37 (m, 1H), 7.22 (t, J=74.8 Hz, 1H).

### Example 139D

### (2-(2-(difluoromethoxy)phenyl)pyrimidin-4-yl)methanol

To a stirring mixture of Example 139C (2.272 g) in tetrahydrofuran (56.8 mL) was added sodium borohydride (0.687 g) in one portion followed by 15 mL of methanol. The resulting mixture was stirred for 30 minutes and carefully quenched by slow addition of 60 mL of saturated aqueous ammonium chloride solution. The mixture obtained was stirred for 15 minutes, poured into a separatory funnel, diluted with water, and extracted with two portions of ethyl acetate. The combined organic layers were dried over anhydrous magnesium sulfate, filtered and concentrated onto silica gel. Purification by flash chromatography on a CombiFlash® Teledyne Isco system using a Teledyne Isco RediSep® Rf gold 80 g silica gel column (eluting with 30-100% ethyl acetate in heptanes) provided the title compound. LC/MS (APCI) m/z 253.3 (M+H)⁺.

### Example 139E

### tert-butyl (7R,16R,21S)-19-chloro-1-(4-fluorophenyl)-10-hydroxy-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

A 20 mL Barnstead Hastelloy C reactor was charged with palladium on carbon (0.55 g, 5% weight palladium, wet). A solution of Example 136N in tetrahydrofuran (2.5 mL) was added and the reactor was purged with argon. The mixture was stirred at 1600 rotations per minute under 50 psi of hydrogen at 25° C. for 48 hours. The mixture was filtered, concentrated under reduced pressure and purified by flash column chromatography on a Teledyne Isco Combiflash Rf instrument using a Teledyne Isco RediSep® Gold 40 g column eluting with a 0-10% methanol/dichlormethane gradient to yield the title compound. ¹H NMR (400 MHz, dimethylsulfoxide-d₆) δ ppm 9.03 (s, 1H), 8.67 (s, 1H), 7.32-7.04 (m, 7H), 6.88 (d, 1H), 6.78-6.51 (m, 2H), 5.91 (dd, 1H), 5.33 (d, 1H), 4.43-4.32 (m, 2H), 4.24 (dd, 1H), 3.65 (dd, 1H), 2.57 (d, 1H), 2.53-2.47 (m, 3H), 2.36-2.25 (m, 8H), 2.24 (s, 3H), 2.10 (s, 3H), 1.01 (s, 9H). MS (ESI+) m/z 761.5 (M+H)⁺.

### Example 139F

### tert-butyl (7R,16R,21S)-19-chloro-10-({2-[2-(difluoromethoxy)phenyl]pyrimidin-4-yl}methoxy)-1-(4-fluorophenyl)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

A 4 mL vial, equipped with stir bar, was charged with, Example 139D (27.2 mg), Example 139E (41 mg) and triphenylphosphine (29.7 mg). The vial was capped with a septa and evacuated and backfilled with nitrogen twice. Toluene (539 μL) was added and after all the reagents completely dissolved the mixture was cooled to 0° C. with an ice bath. Next, (E)-di-tert-butyl diazene-1,2-dicarboxylate (24.80 mg) was added in one portion, and the vial was capped with a septa and evacuated and backfilled with nitrogen twice again. The mixture was stirred at 0° C. for 10 minutes, the cooling bath was removed, and the mixture allowed to stir for 16 hours. The mixture was concentrated onto silica gel, and purification by flash chromatography on a CombiFlash® Teledyne Isco system using a Teledyne Isco RediSep® Rf gold 12 g silica gel column (eluting with 0-10% methanol in dichloromethane) afforded the title compound. LC/MS (APCI) m/z 995.3 (M+H)⁺.

### Example 139G

### (7R,16R,21S)-19-chloro-10-({2-[2-(difluoromethoxy)phenyl]pyrimidin-4-yl}methoxy)-1-(4-fluorophenyl)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid

To a solution of Example 139F (38 mg) in dichloromethane (382 μL) was added trifluoroacetic acid (382 μL). The mixture was stirred at ambient for 5 hours, concentrated and purified directly by reverse phase prep LC using a Gilson 2020 system (Luna, C-18, 250×50 mm column, Mobile phase A: 0.1% trifluoroacetic acid in water; B: acetonitrile; 5-75% B to A gradient at 70 mL/minute) to afford the title compound. ¹H NMR (400 MHz, dimethyl sulfoxide-d₆) δ ppm 8.95 (d, J=5.2 Hz, 1H), 8.75 (s, 1H), 7.89 (dd, J=7.8, 1.8 Hz, 1H), 7.60 (td, J=7.7, 2.0 Hz, 2H), 7.45 (t, J=7.5 Hz, 1H), 7.39-6.95 (m, 9H), 6.92-6.79 (m, 2H), 6.16 (dd, J=5.3, 3.0 Hz, 1H), 5.67 (d, J=2.7 Hz, 1H), 5.20 (q, J=15.2 Hz, 2H), 4.58 (q, J=6.7 Hz, 1H), 4.47 (d, J=13.0 Hz, 1H), 4.36 (dd, J=13.2, 8.4 Hz, 1H), 3.87 (dd, J=17.0, 5.3 Hz, 1H), 3.67-3.46 (m, 2H), 3.16-2.95 (m, 2H), 2.95-2.63 (m, 7H), 2.48-2.31 (m, 2H), 2.22 (s, 3H). LC/MS (APCI) m/z 932.2 (M+H)⁺.

### Example 140

### (7R,16R,21S)-19-chloro-1-(4-fluorophenyl)-10-({2-[2-(methoxymethyl)phenyl]pyrimidin-4-yl}methoxy)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid

### Example 140A

### (2-(2-(methoxymethyl)phenyl)pyrimidin-4-yl)methanol

A mixture of (2-chloropyrimidin-4-yl)methanol (0.50 g), (2-(methoxymethyl)phenyl)boronic acid (0.746 g) and tetrakis(triphenylphosphine)palladium(0) (0.20 g) in tetrahydrofuran (22 mL) and saturated aqueous sodium bicarbonate solution (12 mL) was heated to 75° C. under an atmosphere of nitrogen overnight. The reaction was cooled, diluted with ethyl acetate (75 mL), and washed with water (50 mL) and brine (50 mL). The organic layer was dried over magnesium sulfate, filtered and concentrated. The residue was loaded onto silica gel (Teledyne Isco RediSep® Rf gold 80 g) and was eluted using a gradient of 5-75% heptanes/ethyl acetate. The desired fractions were concentrated to give the title compound. ¹H NMR (400 MHz, chloroform-d) δ ppm 8.79 (d, J=5.0 Hz, 1H), 7.98 (d, J=7.7 Hz, 1H), 7.62 (d, J=7.1 Hz, 1H), 7.49 (td, J=7.6, 7.5, 1.5 Hz, 1H), 7.43 (td, J=7.5, 7.4, 1.5 Hz, 1H), 7.20 (d, J=5.2 Hz, 1H), 4.83 (s, 2H), 4.82 (d, J=5.1 Hz, 2H), 3.70 (t, J=5.1, 5.1 Hz, 1H), 3.35 (s, 3H). MS (ESI) m/z 253.0 (M+Na)⁺.

### Example 140B

### tert-butyl (7R,16R,21S)-19-chloro-1-(4-fluorophenyl)-10-({2-[2-(methoxymethyl)phenyl]pyrimidin-4-yl}methoxy)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

To a mixture of Example 140A (0.012 g), Example 139E (0.020 g) and triphenylphosphine (0.014 g) in toluene (0.263 mL) under nitrogen at 0° C. was added di-tert-butyl azodicarboxylate (0.012 g). The reaction was allowed to warm to room temperature and was stirred for 6 hours. The reaction mixture was loaded onto silica gel (Teledyne Isco RediSep® Rf gold 4 g) and was eluted using a gradient of 0.5-10% methanol/dichloromethane. Product containing fractions were pooled and concentrated from ether to give the title compound. MS (ESI) m/z 973.3 (M+H)⁺.

### Example 140C

### (7R,16R,21S)-19-chloro-1-(4-fluorophenyl)-10-({2-[2-(methoxymethyl)phenyl]pyrimidin-4-yl}methoxy)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid

To a solution of Example 140B (0.018 g) in dichloromethane (0.2 mL) was added trifluoroacetic acid (200 μL) and the reaction was stirred at room temperature. After 6 hours, the reaction was concentrated and dissolved in N,N-dimethylformamide (1 mL) and water (1 mL). The resulting solution was purified by Prep HPLC using a Gilson 2020 system (Luna column, 250×50 mm, flow 70 mL/minutes) using a gradient of 5-75% acetonitrile water over 30 minutes. The product containing fractions were lyophilized to give the title compound. ¹H NMR (400 MHz, dimethyl sulfoxide-d₆) δ ppm 8.94 (d, 1H), 8.75 (s, 1H), 7.93 (dd, 1H), 7.60 (d, 1H), 7.55 (d, 1H), 7.52 (td, 1H), 7.45 (td, 1H), 7.23-7.17 (m, 4H), 7.15 (d, 2H), 6.97 (d, 1H), 6.92 (d, 1H), 6.84 (dd, 1H), 6.17 (dd, 1H), 5.68 (d, 1H), 5.22 (q, 2H), 4.83 (s, 2H), 4.61 (q, 1H), 4.47 (d, 1H), 4.36 (dd, 1H), 3.88 (dd, 1H), 3.39 (d, 3H), 3.23 (s, 3H), 3.05 (s, 4H), 2.92 (dd, 2H), 2.79 (s, 3H), 2.75 (d, 2H), 2.22 (s, 3H). MS (ESI) m/z 917.3 (M+H)⁺.

### Example 141

### (7R,20S)-18-chloro-1-(4-fluorophenyl)-19-methyl-15-[2-(4-methylpiperazin-1-yl)ethyl]-10-({2-[(2R)-oxan-2-yl]pyrimidin-4-yl}methoxy)-7,8,15,16-tetrahydro-14H-17,20-etheno-13,9-(metheno)-6-oxa-2-thia-3,5,15-triazacyclooctadeca[1,2,3-cd]indene-7-carboxylic acid

### Example 141A

### tetrahydro-2H-pyran-2-carboxamide

The title compound was prepared by substituting tetrahydro-2H-pyran-2-carboxylic acid for tetrahydrofuran-3-carboxylic acid in Example 131A. MS (DCI) m/z 130.0 (M+H)⁺.

### Example 141B

### methyl tetrahydro-2H-pyran-2-carbimidate

The title compound was prepared by substituting Example 141A for Example 131A in Example 131B.

### Example 141C

### tetrahydro-2H-pyran-2-carboximidamide, hydrochloride salt

The title compound was prepared by substituting Example 141B for Example 131B in Example 131C. MS (DCI) m/z 128.8 (M+H)⁺.

### Example 141D

### 4-(dimethoxymethyl)-2-(tetrahydro-2H-pyran-2-yl)pyrimidine

The title compound was prepared by substituting Example 141C for Example 65B in Example 65C. MS (DCI) m/z 239.0 (M+H)⁺.

### Example 141E

### (2-(tetrahydro-2H-pyran-2-yl)pyrimidin-4-yl)methanol

The title compound was prepared by substituting Example 141D for Example 65C in Example 65D. MS (DCI) m/z 195.0 (M+H)⁺.

### Example 141F

### (R*)-(2-(tetrahydro-2H-pyran-2-yl)pyrimidin-4-yl)methanol

The title compound was prepared by substituting Example 141E for Example 131E in Example 131F. The absolute stereochemistry was arbitrarily assigned. MS (DCI) m/z 195.0 (M+H)⁺.

### Example 141G

### (S*)-(2-(tetrahydro-2H-pyran-2-yl)pyrimidin-4-yl)methanol

The title compound was prepared during the chromatography procedure described in Example 141F. The absolute stereochemistry was arbitrarily assigned. MS (DCI) m/z 181.0 (M+H)⁺.

### Example 141H

### (R*)-(2-(tetrahydro-2H-pyran-2-yl)pyrimidin-4-yl)methyl methanesulfonate

The title compound was prepared by substituting Example 141F for Example 89B in Example 89C. MS (DCI) m/z 273.0 (M+H)⁺.

### Example 141I

### ethyl (7R,20S)-18-chloro-1-(4-fluorophenyl)-19-methyl-15-[2-(4-methylpiperazin-1-yl)ethyl]-10-({2-[(2R*)-oxan-2-yl]pyrimidin-4-yl}methoxy)-7,8,15,16-tetrahydro-14H-17,20-etheno-13,9-(metheno)-6-oxa-2-thia-3,5,15-triazacyclooctadeca[1,2,3-cd]indene-7-carboxylate

The title compound was prepared by substituting Example 141H for Example 131H in Example 131I. MS (ESI) m/z 906.2 (M+H)⁺.

### Example 141J

### (7R,20S)-18-chloro-1-(4-fluorophenyl)-19-methyl-15-[2-(4-methylpiperazin-1-yl)ethyl]-10-({2-[(2R*)-oxan-2-yl]pyrimidin-4-yl}methoxy)-7,8,15,16-tetrahydro-14H-17,20-etheno-13,9-(metheno)-6-oxa-2-thia-3,5,15-triazacyclooctadeca[1,2,3-cd]indene-7-carboxylic acid

The title compound was prepared by substituting Example 141I for Example 65N in Example 65O. ¹H NMR (500 MHz, dimethylsulfoxide-d₆) δ ppm 8.65 (s, 1H), 8.57 (d, 1H), 7.50 (d, 1H), 7.27 (d, 1H), 7.24 (m, 2H), 7.15 (m, 4H), 6.79 (d, 1H), 6.47 (s, 1H), 5.91 (dd, 1H), 5.15 (d, 1H), 5.05 (d, 1H), 4.41 (dd, 1H), 4.26 (v br s, 2H), 4.08 (v br s, 2H), 3.96 (br m, 1H), 3.52 (m, 5H), 3.18 (m, 4H), 3.05 (m, 4H), 2.78 (s, 3H), 1.87 (m, 1H), 1.75 (m, 2H), 1.74 (s, 3H), 1.63 (m, 1H), 1.55 (m, 2H). MS (ESI) m/z 878.5 (M+H)⁺.

### Example 142

### (7R,20S)-18-chloro-1-(4-fluorophenyl)-19-methyl-15-[2-(4-methylpiperazin-1-yl)ethyl]-10-({2-[(2S)-oxan-2-yl]pyrimidin-4-yl}methoxy)-7,8,15,16-tetrahydro-14H-17,20-etheno-13,9-(metheno)-6-oxa-2-thia-3,5,15-triazacyclooctadeca[1,2,3-cd]indene-7-carboxylic acid

### Example 142A

### (S*)-(2-(tetrahydro-2H-pyran-2-yl)pyrimidin-4-yl)methyl methanesulfonate

The title compound was prepared by substituting Example 141G for Example 89B in Example 89C. MS (DCI) m/z 273.0 (M+H)⁺.

### Example 142B

### ethyl (7R,20S)-18-chloro-1-(4-fluorophenyl)-19-methyl-15-[2-(4-methylpiperazin-1-yl)ethyl]-10-({2-[(2S*)-oxan-2-yl]pyrimidin-4-yl}methoxy)-7,8,15,16-tetrahydro-14H-17,20-etheno-13,9-(metheno)-6-oxa-2-thia-3,5,15-triazacyclooctadeca[1,2,3-cd]indene-7-carboxylate

The title compound was prepared by substituting Example 142A for Example 131H in Example 131I. MS (ESI) m/z 906.2 (M+H)⁺.

### Example 142C

### (7R,20S)-18-chloro-1-(4-fluorophenyl)-19-methyl-15-[2-(4-methylpiperazin-1-yl)ethyl]-10-({2-[(2S*)-oxan-2-yl]pyrimidin-4-yl}methoxy)-7,8,15,16-tetrahydro-14H-17,20-etheno-13,9-(metheno)-6-oxa-2-thia-3,5,15-triazacyclooctadeca[1,2,3-cd]indene-7-carboxylic acid

The title compound was prepared by substituting Example 142B for Example 65N in Example 65O. ¹H NMR (500 MHz, dimethylsulfoxide-d₆) δ ppm 8.66 (s, 1H), 8.58 (d, 1H), 7.51 (d, 1H), 7.29 (d, 1H), 7.22 (m, 4H), 7.15 (m, 2H), 6.80 (d, 1H), 6.46 (s, 1H), 5.92 (dd, 1H), 5.16 (d, 1H), 5.05 (d, 1H), 4.41 (dd, 1H), 4.32 (v br m, 2H), 4.16 (v br s, 2H), 3.97 (br m, 1H), 3.54 (m, 5H), 3.19 (m, 4H), 3.05 (m, 4H), 2.80 (s, 3H), 1.86 (m, 1H), 1.76 (m, 2H), 1.75 (s, 3H), 1.65 (m, 1H), 1.55 (m, 2H). MS (ESI) m/z 878.5 (M+H)⁺.

### Example 143

### (7R,15S,21S)-19-chloro-1-(4-fluorophenyl)-10-{[2-(2-methoxyphenyl)pyrimidin-4-yl]methoxy}-20-methyl-15-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid

### Example 143A

### (S)-4-((4-bromo-2-chloro-3-methylphenoxy)methyl)-2,2-dimethyl-1,3-dioxolane

Triphenylphosphine (10.45 g) and N,N,N′,N′-tetramethylazodicarboxamide (6.61 g) were stirred in 220 mL tetrahydrofuran at 0° C. for 10 minutes, and (S)-(2,2-dimethyl-1,3-dioxolan-4-yl)methanol (4.14 g) and 4-bromo-2-chloro-3-methylphenol (6.3 g) were added and the reaction was stirred overnight. Ether (100 mL) was added, 150 mL heptanes were added slowly, and the mixture was stirred another 20 minutes. The mixture was filtered, and ethyl acetate was added to the organic layer, which was then washed twice with 1M aqueous NaOH, washed with brine, dried over Na₂SO₄, filtered and concentrated. The crude material was chromatographed on silica gel using 10% ethyl acetate in heptanes to give the title compound. MS (APCI) m/z 335.1 (M+H)⁺.

### Example 143B

### (R)-3-(4-bromo-2-chloro-3-methylphenoxy)propane-1,2-diol

To a stirring mixture of Example 143A (8.6 g) in 100 mL methanol was slowly added 1M aqueous HCl (32.0 mL), and the reaction was stirred overnight. The mixture was concentrated to remove most of the methanol, and carefully poured into 150 mL of saturated aqueous NaHCO₃ solution. The aqueous solution was extracted three times with ethyl acetate. The extracts were washed with brine, dried over Na₂SO₄, filtered and concentrated to give the title compound. ¹H NMR (dimethylsulfoxide-d₆) δ ppm 7.51 (d, 1H), 6.99 (d, 1H), 4.97 (d, 1H), 4.66 (t, 1H), 4.04 (dd, 1H), 3.96 (d, 1H), 3.80 (m, 1H), 3.47 (m, 2H), 2.44 (s, 3H).

### Example 143C

### (S)-1-(4-bromo-2-chloro-3-methylphenoxy)-3-((tert-butyldimethylsilyl)oxy)propan-2-ol

DMAP (4-dimethylaminopyridine, 0.076 g) was added to a mixture of Example 143B (3.7 g), TBS-Cl (tert-butyldimethylchlorosilane, 1.887 g), and triethylamine (1.745 mL) in 50 mL N,N-dimethylformamide, and the reaction was stirred for 4 hours. The reaction was poured into 400 mL water and was extracted three times with ethyl acetate. The combined extracts were washed three times with water, washed with brine, dried over Na₂SO₄, filtered and concentrated. The crude material was chromatographed on silica gel using 10% ethyl acetate in heptanes to give the title compound. MS (APCI) m/z 409.9 (M+H)⁺.

### Example 143D

### (S)-1-((tert-butyldimethylsilyl)oxy)-3-(2-chloro-3-methyl-4-(4,4,5,5-tetramethyl-1,3,2-dioxaborolan-2-yl)phenoxy)propan-2-ol

Example 143C (3.3 g), bis(pinacolato)diboron (2.454 g), PdCl₂dppf ([1,1′-bis(diphenylphosphino)ferrocene]dichloropalladium(II), 0.329 g) and potassium acetate (1.581 g) were taken up in 40 mL dioxane, and the mixture was subjected to several vacuum/nitrogen cycles, and heated to 90° C. overnight. The mixture was cooled, poured into ethyl acetate, washed with water and brine, dried over Na₂SO₄, filtered and concentrated. The crude material was chromatographed on silica gel using 1-10% ethyl acetate in heptanes to give the title compound. MS (APCI) m/z 457.1 (M+H)⁺.

### Example 143E

### (2R)-ethyl 3-(5-((tert-butyldimethylsilyl)oxy)-2-((2-(2-methoxyphenyl)pyrimidin-4-yl)methoxy)phenyl)-2-((5-((1S)-4-((S)-3-((tert-butyldimethylsilyl)oxy)-2-hydroxypropoxy)-3-chloro-2-methylphenyl)-6-(4-fluorophenyl)thieno[2,3-d]pyrimidin-4-yl)oxy)propanoate

Example 68C (2.96 g), Example 143D (2.08 g), potassium phosphate (1.858 g) and bis(di-tert-butyl(4-dimethylaminophenyl)phosphine)dichloropalladium (0.124 g) were placed in a 25 mL flask. The mixture was degassed and purged with nitrogen. Tetrahydrofuran (6 mL) and water (1.5 mL) were added via syringe and the solution was repeatedly degassed and purged with nitrogen. The reaction was stirred overnight. The crude material was chromatographed on silica gel using 1-50% ethyl acetate in heptanes to give the title compound. MS (APCI) m/z 1095.2 (M+H)⁺.

### Example 143F

### (2R)-ethyl 2-((5-((1S)-3-chloro-4-((R)-2,3-dihydroxypropoxy)-2-methylphenyl)-6-(4-fluorophenyl)thieno[2,3-d]pyrimidin-4-yl)oxy)-3-(5-hydroxy-2-((2-(2-methoxyphenyl)pyrimidin-4-yl)methoxy)phenyl)propanoate

Example 143E (1.89 g) was taken up in 50 mL tetrahydrofuran, and 1M TBAF (tetra-N-butylammonium fluoride) in tetrahydrofuran (3.65 mL) was added. The reaction was stirred for 10 minutes. The reaction was quenched with saturated aqueous NaH₂PO₄ solution, and extracted with ethyl acetate. The organic layer was washed with brine, and concentrated. The crude material was chromatographed on silica gel using 10-100% ethyl acetate in heptanes to give the title compound. MS (APCI) m/z 867.1 (M+H)⁺.

### Example 143G

### (2R)-ethyl 2-((5-((1S)-4-((S)-3-((tert-butyldimethylsilyl)oxy)-2-hydroxypropoxy)-3-chloro-2-methylphenyl)-6-(4-fluorophenyl)thieno[2,3-d]pyrimidin-4-yl)oxy)-3-(5-hydroxy-2-((2-(2-methoxyphenyl)pyrimidin-4-yl)methoxy)phenyl)propanoate

tert-Butyldimethylsilyl trifluoromethanesulfonate (132 μL) was added to Example 143F (500 mg) and 2,6-lutidine (101 μL) in 6 mL dichloromethane at −40° C. The reaction was stirred for 20 minutes. The crude mixture was directly chromatographed on silica gel using 10-100% ethyl acetate in heptanes to give the title compound. MS (APCI) m/z 981.3 (M+H)⁺.

### Example 143H

### ethyl (7R,15S,21S)-19-chloro-1-(4-fluorophenyl)-15-(hydroxymethyl)-10-{[2-(2-methoxyphenyl)pyrimidin-4-yl]methoxy}-20-methyl-7,8,15,16-tetrahydro-18,21-etheno-13,9-(metheno)-6,14,17-trioxa-2-thia-3,5-diazacyclononadeca[1,2,3-cd]indene-7-carboxylate

To a solution of triphenylphosphine (524 mg) in 5 mL tetrahydrofuran at 0° C. was added N,N,N′,N′-tetramethylazodicarboxamide (345 mg), and the reaction was stirred for 10 minutes. A solution of Example 143G (1160 mg) in 6 mL tetrahydrofuran was added, and the reaction was stirred at 30° C. for two days. The crude mixture was directly chromatographed on silica gel using 10-100% ethyl acetate in heptanes to give the silylated product. The material was taken up in 10 mL tetrahydrofuran, and 1M TBAF (tetra-N-butylammonium fluoride) in tetrahydrofuran (1182 μL) was added. The reaction was stirred for 5 minutes. The reaction was quenched with saturated aqueous NaH₂PO₄ solution, and extracted with ethyl acetate. The organic layer was washed with brine, and concentrated. The crude material was purified by reverse phase using a 20-90% gradient of acetonitrile in water (with 0.1% trifluoroacetic acid) over 45 minutes on a Grace Reveleris equipped with a Luna column: C18(2), 100 A, 250×50 mm to isolate the title compound. MS (APCI) m/z 849.3 (M+H)⁺.

### Example 143I

### ethyl (7R,15R,21S)-19-chloro-1-(4-fluorophenyl)-10-{[2-(2-methoxyphenyl)pyrimidin-4-yl]methoxy}-20-methyl-15-{[(4-methylbenzene-1-sulfonyl)oxy]methyl}-7,8,15,16-tetrahydro-18,21-etheno-13,9-(metheno)-6,14,17-trioxa-2-thia-3,5-diazacyclononadeca[1,2,3-cd]indene-7-carboxylate

TsCl (p-toluenesulfonyl chloride, 32.1 mg) was added to a solution of Example 143H (130 mg) and triethylamine (32.0 μL) in 1 mL dichloromethane and the reaction was stirred for four days total. The crude mixture was chromatographed on silica gel using 10-100% ethyl acetate in heptanes to give the title compound. MS (APCI) m/z 1003.1 (M+H)⁺.

### Example 143J

### ethyl (7R,15S,21S)-19-chloro-1-(4-fluorophenyl)-10-{[2-(2-methoxyphenyl)pyrimidin-4-yl]methoxy}-20-methyl-15-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

Example 143I (30 mg) and 1-methylpiperazine (120 mg) were taken up in 1 mL N,N-dimethylformamide and the mixture was stirred at 35° C. for 6 days. The crude material was purified by reverse phase using a 20-90% gradient of acetonitrile in water (with 0.1% trifluoroacetic acid) over 40 minutes on a Grace Reveleris equipped with a Luna column: C18(2), 100 A, 250×50 mm to isolate the title compound. MS (APCI) m/z 931.5 (M+H)⁺.

### Example 143K

### (7R,15S,21S)-19-chloro-1-(4-fluorophenyl)-10-{[2-(2-methoxyphenyl)pyrimidin-4-yl]methoxy}-20-methyl-15-[(4-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid

A 1M aqueous solution of lithium hydroxide (215 μL) was added to Example 143J (50 mg) in 0.8 mL tetrahydrofuran and 0.3 mL methanol and the reaction was stirred overnight. The crude material was purified by reverse phase using a 10-85% gradient of acetonitrile in water (with 0.1% trifluoroacetic acid) over 40 minutes on a Grace Reveleris equipped with a Luna column: C18(2), 100 A, 250×50 mm to isolate the title compound. ¹H NMR (dimethylsulfoxide-d₆) δ ppm 9.55 (br s, 1H), 8.88 (d, 1H), 8.73 (d, 1H), 7.63-7.43 (m, 4H), 7.32-7.16 (m, 6H), 7.07 (dd, 1H), 6.95 (d, 1H), 6.89 (s, 2H), 6.19 (s, 1H), 5.64 (s, 1H), 5.17 (q, 2H), 4.67 (dd, 1H), 4.52 (d, 1H), 4.32 (d, 1H), 3.83 (dd, 1H), 3.78 (s, 3H), 3.11 (m, 4H), 2.89 (m, 2H), 2.78 (s, 3H), 2.74 (m, 2H), 2.46 (m, 2H), 2.19 (s, 3H). MS (APCI) m/z 904.4 (M+H)⁺.

### Example 144

### (7R,16R,21S)-19-chloro-10-{[2-(5-fluoro-2-methoxyphenyl)pyrimidin-4-yl]methoxy}-1-(4-fluorophenyl)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid

### Example 144A

### (2-(5-fluoro-2-methoxyphenyl)pyrimidin-4-yl)methanol

To a solution of (5-fluoro-2-methoxyphenyl)boronic acid (1.71 g) and (2-chloropyrimidin-4-yl)methanol (1.45 g) in tetrahydrofuran (30 mL) was added Pd(Ph₃P)₄ (tetrakis(triphenylphosphine)palladium(0), 580 mg) and a solution of aqueous saturated sodium bicarbonate (40 mL). The mixture was stirred under nitrogen at 70° C. overnight. After cooling to ambient temperature, the solvent was evaporated under vacuum and the residue was diluted with water (60 mL) and ethyl acetate (300 mL). The organic layer was separated and washed with water and brine, dried over anhydrous sodium sulfate, filtered and concentrated under reduced pressure. The crude material was purified by flash column chromatography on a Teledyne Isco Combiflash Rf instrument using a Teledyne Isco RediSep® Gold 80 g column eluting with a 5-95% ethyl acetate/heptanes gradient to give the title compound. ¹H NMR (501 MHz, Chloroform-d) δ ppm 8.80 (d, 1H), 7.50 (dd, 1H), 7.25 (dt, 1H), 7.13 (ddd, 1H), 6.98 (dd, 1H), 4.81 (d, 2H), 3.85 (s, 3H), 3.67 (t, 1H). LC/MS (ESI) 235.07 (M+H)⁺.

### Example 144B

### tert-butyl (7R,16R,21S)-19-chloro-10-{[2-(5-fluoro-2-methoxyphenyl)pyrimidin-4-yl]methoxy}-1-(4-fluorophenyl)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

An oven dried 1 dram vial equipped with a magnetic stir bar was charged with Example 139E (36 mg), and Example 144A (19 mg). Toluene (0.5 mL) was added and the mixture was stirred. Triphenylphosphine (25 mg) was added followed by di-tert-butyl azodicarboxylate (22 mg). The reaction was stirred for 3 days at which point the reaction mixture was loaded onto a small filtration flask loaded with silica gel (10 g). The filtration plug was eluted with 30% (3:1 ethyl acetate/ethanol)/heptanes (30 mL). The initial filtrate was discarded and the silica plug was then eluted with 10% methanol/dichloromethane (40 mL). The filtrate was concentrated under reduced pressure and the crude material was repurified by flash column chromatography on a Teledyne Isco Combiflash Rf instrument using a Teledyne Isco RediSep® Gold 80 g column eluting with a 0-10% methanol/dichloromethane gradient to give the title compound. ¹H NMR (500 MHz, dimethylsulfoxide-d₆) δ ppm 8.90 (d, 1H), 8.67 (s, 1H), 7.56 (d, 1H), 7.32 (dd, 1H), 7.26 (td, 1H), 7.19-7.09 (m, 6H), 6.90 (d, 1H), 6.88 (d, 1H), 6.82 (dd, 1H), 5.53 (d, H), 5.14 (d, 1H), 5.06 (d, 1H), 4.44 (q, Hz, 1H), 4.39 (d, 1H), 4.32 (dd, 1H), 3.80 (dd, 1H), 3.70 (s, 3H), 3.11-2.79 (m, 4H), 2.78-2.62 (m, 6H), 2.19 (s, 3H), 0.94 (s, 9H). MS (ESI) m/z 977.2 (M+H)⁺.

### Example 144C

### (7R,16R,21S)-19-chloro-10-{[2-(5-fluoro-2-methoxyphenyl)pyrimidin-4-yl]methoxy}-1-(4-fluorophenyl)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid

A 1 dram vial was charged with Example 144B and was equipped with a magnetic stir bar and septum screw cap. Dichloromethane (0.2 mL) and trifluoroacetic acid (0.2 mL) were sequentially added and the reaction mixture was stirred for 5 hours. The volatiles were concentrated under a stream of nitrogen and the residue was purified by preparative reversed phase high pressure liquid chromatography on a Gilson PLC 2250 system equipped with a Phenomenex® Luna™ C18(2) 50×250 mm column eluting with 10-90% acetonitrile/(0.1% aqueous trifluoroacetic acid) gradient. The volatiles were removed by lyophilization to give the title compound as the bis-trifluoroacetic acid salt. ¹H NMR (501 MHz, dimethylsulfoxide-d₆) δ ppm 8.91 (d, 1H), 8.75 (s, 1H), 7.56 (d, 1H), 7.40 (dd, 1H), 7.33 (ddd, 1H), 7.24-7.17 (m, 5H), 7.16 (d, 1H), 6.97 (d, 1H), 6.91 (d, J=9.1 Hz, 1H), 6.84 (dd, 1H), 6.16 (dd, 1H), 5.67 (d, 1H), 5.22 (d, 1H), 5.15 (d, 1H), 4.60 (q, 1H), 4.47 (d, 1H), 4.37 (dd, 1H), 3.87 (dd, 1H), 3.77 (s, 3H), 3.44-3.30 (m, 2H), 3.23-2.97 (m, 4H), 2.90 (dd, 1H), 2.79 (s, 3H), 2.78-2.71 (m, 2H), 2.23 (s, 3H). MS (ESI) m/z 921.2 (M+H)⁺.

### Example 145

### (7R,16R,21S)-19-chloro-1-(4-fluorophenyl)-20-methyl-16-[(4-methylpiperazin-1-yl)methyl]-10-({2-[(2S)-oxolan-2-yl]pyrimidin-4-yl}methoxy)-7,8,15,16-tetrahydro-18,21-etheno-13,9-(metheno)-6,14,17-trioxa-2-thia-3,5-diazacyclononadeca[1,2,3-cd]indene-7-carboxylic acid

### Example 145A

### tert-butyl (7R,16R,21S)-19-chloro-1-(4-fluorophenyl)-20-methyl-16-[(4-methylpiperazin-1-yl)methyl]-10-({2-[(2S)-oxolan-2-yl]pyrimidin-4-yl}methoxy)-7,8,15,16-tetrahydro-18,21-etheno-13,9-(metheno)-6,14,17-trioxa-2-thia-3,5-diazacyclononadeca[1,2,3-cd]indene-7-carboxylate

The title compound was prepared by substituting Example 85F for Example 144A in Example 144B. MS (ESI) m/z 923.2 (M+H)⁺.

### Example 145B

### (7R,16R,21S)-19-chloro-1-(4-fluorophenyl)-20-methyl-16-[(4-methylpiperazin-1-yl)methyl]-10-({2-[(2S)-oxolan-2-yl]pyrimidin-4-yl}methoxy)-7,8,15,16-tetrahydro-18,21-etheno-13,9-(metheno)-6,14,17-trioxa-2-thia-3,5-diazacyclononadeca[1,2,3-cd]indene-7-carboxylic acid

The title compound was prepared by substituting Example 145A for Example 144B in Example 144C. ¹H NMR (500 MHz, dimethylsulfoxide-d₆) δ ppm 8.79 (d, 1H), 8.74 (s, 1H), 7.48 (d, 1H), 7.20 (m, 4H), 7.15 (d, 1H), 6.96 (d, 1H), 6.85 (m, 2H), 6.16 (m, 1H), 5.66 (d, 1H), 5.18 (d, 1H), 5.10 (d, 1H), 4.96 (dd, 1H), 4.59 (m, 1H), 4.46 (d, 1H), 4.36 (m, 1H), 4.00 (m, 1H), 3.85 (m, 4H), 3.82 (m, 1H), 3.37 (v br s, 2H), 3.08 (v br s, 2H), 2.89 (d, 2H), 2.80 (s, 3H), 2.76 (br m, 2H), 2.30 (m, 1H), 2.22 (s, 3H), 2.05 (m, 2H), 1.94 (m, 1H). MS (ESI) m/z 867.4 (M+H)⁺.

### Example 146

### (7R,16R,21S)-19-chloro-1-(4-fluorophenyl)-10-({2-[2-(methanesulfonyl)phenyl]pyrimidin-4-yl}methoxy)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid

### Example 146A

### tert-butyl (7R,16R,21S)-19-chloro-1-(4-fluorophenyl)-10-({2-[2-(methanesulfonyl)phenyl]pyrimidin-4-yl}methoxy)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

The title compound was prepared as described in Example 140B substituting Example 130C for Example 140A. MS (ESI) m/z 1007.2 (M+H)⁺.

### Example 146B

### (7R,16R,21S)-19-chloro-1-(4-fluorophenyl)-10-({2-[2-(methanesulfonyl)phenyl]pyrimidin-4-yl}methoxy)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid

The title compound was prepared as described in Example 140C, substituting Example 146A for Example 140B. ¹H NMR (501 MHz, Chloroform-d) δ ppm 8.87 (d, J=5.1 Hz, 1H), 8.63 (s, 1H), 8.21 (dd, 1H), 7.82-7.72 (m, 2H), 7.71-7.65 (m, 2H), 7.16 (d, 1H), 7.13-7.07 (m, 2H), 6.99-6.89 (m, 3H), 6.81-6.64 (m, 2H), 6.07 (dd, 1H), 5.78 (d, 1H), 5.14 (s, 2H), 4.64 (d, 1H), 4.45 (dd, 1H), 4.36 (dd, 1H), 3.89 (dd, 1H), 3.52 (s, 3H), 3.48 (q, 2H), 2.90 (dd, 1H), 2.77 (dd, 1H), 2.62-2.35 (m, 8H), 2.29 (s, 3H), 2.24 (s, 3H), 1.21 (t, 2H). MS (ESI) m/z 951.0 (M+H)⁺.

### Example 147

### (7R,16R,21S)-19-chloro-1-(4-fluorophenyl)-20-methyl-16-[(4-methylpiperazin-1-yl)methyl]-10-({2-[(2S)-oxan-2-yl]pyrimidin-4-yl}methoxy)-7,8,15,16-tetrahydro-18,21-etheno-13,9-(metheno)-6,14,17-trioxa-2-thia-3,5-diazacyclononadeca[1,2,3-cd]indene-7-carboxylic acid

### Example 147A

### tert-butyl (7R,16R,21S)-19-chloro-1-(4-fluorophenyl)-20-methyl-16-[(4-methylpiperazin-1-yl)methyl]-10-({2-[(2S)-oxan-2-yl]pyrimidin-4-yl}methoxy)-7,8,15,16-tetrahydro-18,21-etheno-13,9-(metheno)-6,14,17-trioxa-2-thia-3,5-diazacyclononadeca[1,2,3-cd]indene-7-carboxylate

The title compound was prepared by substituting Example 141G for Example 144A in Example 144B. MS (ESI) m/z 937.4 (M+H)⁺.

### Example 147B

### (7R,16R,21S)-19-chloro-1-(4-fluorophenyl)-20-methyl-16-[(4-methylpiperazin-1-yl)methyl]-10-({2-[(2S)-oxan-2-yl]pyrimidin-4-yl}methoxy)-7,8,15,16-tetrahydro-18,21-etheno-13,9-(metheno)-6,14,17-trioxa-2-thia-3,5-diazacyclononadeca[1,2,3-cd]indene-7-carboxylic acid

The title compound was prepared by substituting Example 147A for Example 144B in Example 144C. ¹H NMR (500 MHz, dimethylsulfoxide-d₆) δ ppm 8.79 (d, 1H), 8.74 (s, 1H), 7.49 (d, 1H), 7.19 (m, 4H), 7.14 (d, 1H), 6.96 (d, 1H), 6.86 (d, 1H), 6.83 (m, 1H), 6.14 (m, 1H), 5.65 (d, 1H), 5.18 (d, 1H), 5.11 (d, 1H), 4.58 (m, 1H), 4.47 (m, 2H), 4.36 (m, 1H), 3.97 (m, 1H), 3.83 (dd, 1H), 3.57 (m, 1H), 3.37 (v br s, 2H), 3.07 (v br s, 3H), 2.88 (d, 2H), 2.80 (s, 3H), 2.73 (br m, 2H), 2.39 (m, 2H), 2.22 (s, 3H), 1.82 (m, 3H), 1.66 (m, 1H), 1.56 (m, 2H). MS (ESI) m/z 881.2 (M+H)⁺.

### Example 148

### (7R,16R,21S)-19-chloro-1-(4-fluorophenyl)-10-{[2-(2-hydroxyphenyl)pyrimidin-4-yl]methoxy}-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid

### Example 148A

### 2-(4-(dimethoxymethyl)pyrimidin-2-yl)phenol

2-Hydroxybenzimidamide hydrochloride (2.5 g) was dissolved in ethanol (60 mL). Sodium ethanolate (21% in ethanol, 10.81 mL) was added, followed by Example 100A (2.76 g). The reaction was stirred at 70° C. for 16 hours. The solvent was removed by rotary evaporation. The residue was taken up in 50% ethyl acetate in heptanes (100 mL). Saturated aqueous ammonium chloride (20 mL) was added and the layers were separated. The organic layer was washed with water (2×20 mL) and with brine (20 mL). The solution was dried on anhydrous sodium sulfate and filtered. The solvent was removed under vacuum to yield the title compound. ¹H NMR (400 MHz, dimethyl sulfoxide-d₆) δ ppm 13.15 (s, 1H), 9.03 (d, 1H), 8.41 (dd, 1H), 7.55 (d, 1H), 7.44 (td, 1H), 7.01 (dd, 1H), 6.99 (d, 1H), 5.49 (s, 1H), 3.40 (s, 6H). MS (ESI) m/z 245 (M−H)⁻.

### Example 148B

### 2-(4-(hydroxymethyl)pyrimidin-2-yl)phenol

Example 148A (1.5 g) was dissolved in 1,4-dioxane (25 mL). Aqueous hydrogen chloride (2 M, 25 mL) was added and the solution was heated to 50° C. for 16 hours. The solution was cooled to room temperature and further cooled to 0° C. using an ice bath. The pH of the solution was adjusted to eight using concentrated aqueous sodium hydroxide. To the solution was added sodium borohydride (0.461 g) in three portions, five minutes apart. The solution was mixed at 0° C. for two hours. While keeping the reaction at 0° C., 10 mL of ethyl acetate was added, and the mixture was stirred for 10 minutes. The mixture was diluted further with ethyl acetate (20 mL), keeping the reaction at 0° C. Saturated aqueous ammonium chloride (5 mL) was added, and the solution was stirred for 10 minutes. The phases were separated. The pH of the aqueous layer was adjusted to five using 2 M aqueous HCl. The aqueous layer was extracted once with ethyl acetate (20 mL). The organic portions were combined and dried on anhydrous sodium sulfate, and filtered. The mixture was concentrated under vacuum and was purified by flash column chromatography on silica gel using a gradient of 60-80% ethyl acetate in heptanes. The solvent was removed by rotary evaporation to yield the title compound. ¹H NMR (400 MHz, dimethyl sulfoxide-d₆) δ ppm 13.29 (s, 1H), 8.93 (d, 1H), 8.40 (dd, 1H), 7.54 (d, 1H), 7.41 (td, 1H), 6.98-6.94 (m, 2H), 5.78 (t, 1H), 4.69 (d, 2H). MS (ESI) m/z 203 (M+H)⁺.

### Example 148C

### 2-(4-(((tert-butyldimethylsilyl)oxy)methyl)pyrimidin-2-yl)phenol

Example 148B (1000 mg) was dissolved in tetrahydrofuran (12 mL). 1H-Imidazole (741 mg) was added and the solution was cooled to 0° C. tert-Butylchlorodimethylsilane (820 mg) dissolved in tetrahydrofuran (6 mL) was added. The solution was stirred at 0° C. for 5 minutes, and was allowed to warm to room temperature. Additional tetrahydrofuran (10 mL) was added, and the solution was stirred at room temperature for 16 hours. Saturated aqueous ammonium chloride (5 mL) was added. The solution was extracted with ethyl acetate (2×20 mL). The organic extracts were combined and were washed with water (10 mL) and brine (10 mL). The solution was dried over anhydrous sodium sulfate. The solution was concentrated on vacuum and was purified by flash column chromatography on silica gel using a gradient of 20-100% ethyl acetate in heptanes. The solvent was removed by rotary evaporation to provide the title compound. ¹H NMR (400 MHz, dimethyl sulfoxide-d₆) δ ppm 13.21 (s, 1H), 8.95 (d, 1H), 8.38 (dd, 1H), 7.48 (d, 1H), 7.41 (td, 1H), 6.96 (d, 1H), 6.95 (dd, 1H), 4.88 (s, 2H), 0.94 (s, 9H), 0.14 (s, 6H). LC/MS (APCI) m/z 317 (M+H)⁺.

### Example 148D

### tert-butyl (2-(4-(((tert-butyldimethylsilyl)oxy)methyl)pyrimidin-2-yl)phenyl) carbonate

Example 148C (500 mg) was dissolved in tetrahydrofuran (10 mL). Sodium hydride (60% in mineral oil, 69.5 mg) was added, and the solution was stirred at room temperature for five minutes. Di-tert-butyl dicarbonate (379 mg) was added, and the solution was stirred at room temperature for 16 hours. The solvent was removed under vacuum, and the residue was taken up in ethyl acetate (10 mL). Saturated aqueous ammonium chloride (2 mL) and water (0.5 mL) were added. The layers were separated. The organic layer was washed with brine, dried on anhydrous sodium sulfate, and filtered. The solvent was removed under vacuum to yield the title compound. ¹H NMR (400 MHz, dimethyl sulfoxide-d₆) δ ppm 8.91 (d, 1H), 8.11 (dd, 1H), 7.55 (td, 1H), 7.45 (d, 1H), 7.42 (td, 1H), 7.26 (dd, 1H), 4.80 (s, 2H), 1.40 (s, 9H), 0.94 (s, 9H), 0.13 (s, 6H). LC/MS (APCI) m/z 417 (M+H)⁺.

### Example 148E

### tert-butyl (2-(4-(hydroxymethyl)pyrimidin-2-yl)phenyl) carbonate

Example 148D (658 mg) was dissolved in tetrahydrofuran (6 mL). Acetic acid (0.271 mL) was added. Tetrabutylammonium fluoride (1 M in tetrahydrofuran, 3.16 mL) was added. The solution was stirred at room temperature for 30 minutes. The solution was concentrated under vacuum and the crude material was purified by flash column chromatography on silica gel using a gradient of 50-70% ethyl acetate in heptanes. The solvent was removed by rotary evaporation to yield the title compound. ¹H NMR (400 MHz, dimethyl sulfoxide-d₆) δ ppm 8.87 (d, 1H), 8.11 (dd, 1H), 7.54 (td, 1H), 7.50 (d, 1H), 7.41 (td, 1H), 7.25 (dd, 1H), 5.68 (t, 1H), 4.61 (d, 2H), 1.41 (s, 9H). MS (ESI) m/z 303 (M+H)⁺.

### Example 148F

### tert-butyl (7R,16R,21S)-10-[(2-{2-[(tert-butoxycarbonyl)oxy]phenyl}pyrimidin-4-yl)methoxy]-19-chloro-1-(4-fluorophenyl)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

Example 148E (48 mg), Example 139E (60 mg), and triphenylphosphine (43 mg) were dissolved in toluene (0.8 mL). The solution was cooled to 0° C. using an ice bath. (E)-Di-tert-butyl diazene-1,2-dicarboxylate (36 mg) was added. The reaction was allowed to warm to room temperature and stir for 16 hours. Additional Example 148D (48 mg), triphenylphosphine (43 mg) and (E)-di-tert-butyl diazene-1,2-dicarboxylate (36 mg) were added. The reaction was stirred another 24 hours at room temperature. The mixture was purified by flash column chromatography on silica gel using a gradient of 0-10% methanol in dichloromethane. The solvent was removed by rotary evaporation to yield the title compound. ¹H NMR (400 MHz, dimethyl sulfoxide-d₆) δ ppm 8.96 (d, 1H), 8.73 (s, 1H), 8.15 (m, 1H), 7.60 (d, 1H), 7.43 (td, 1H), 7.32-7.26 (m, 3H), 7.24-7.16 (m, 4H), 6.95-6.92 (m, 2H), 6.83 (dd, 1H), 6.08 (dd, 1H), 5.57 (d, 1H), 5.20 (m, 2H), 4.66 (m, 1H), 4.48 (d, 1H), 4.33 (dd, 1H), 3.88 (dd, 2H), 2.82 (m, 2H), 2.35-2.21 (m, 6H), 2.26 (s, 3H), 2.19 (s, 2H), 2.10 (s, 3H), 1.40 (s, 9H), 1.00 (s, 9H). MS (ESI) m/z 1045 (M+H)⁺.

### Example 148G

### (7R,16R,21S)-19-chloro-1-(4-fluorophenyl)-10-{[2-(2-hydroxyphenyl)pyrimidin-4-yl]methoxy}-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid

Example 148F (41 mg) was dissolved in dichloromethane (0.25 mL). Trifluoroacetic acid (0.2 mL) was added and the solution was stirred at room temperature. After five hours, more trifluoroacetic acid (0.2 mL) was added. The reaction was stirred for an additional two hours, and more trifluoroacetic acid (0.1 mL) was added. The reaction was stirred for an additional 1.5 hours, and the solvents were removed under vacuum. The residue was taken up in N,N-dimethylformamide (1 mL) and water (1 mL). The material was purified by reverse phase chromatography using a 30-100% gradient of acetonitrile in water (with 0.1% trifluoroacetic acid) over 40 minutes on a Grace Reveleris equipped with a Luna column: C18(2), 100 A, 250×50 mm. The desired fractions were pooled, frozen and lyophilized to isolate the title compound as the bis trifluoroacetic acid salt. ¹H NMR (400 MHz, dimethyl sulfoxide-d₆) δ ppm 8.98 (d, 1H), 8.75 (s, 1H), 8.42 (dd, 1H), 7.60 (d, 1H), 7.44 (td, 1H), 7.23-7.16 (m, 4H), 7.14 (d, 1H), 7.01-6.91 (m, 4H), 6.83 (dd, 1H), 6.17 (m, 1H), 5.68 (d, 1H), 5.31 (dd, 2H), 4.59 (m, 1H), 4.47 (d, 1H), 4.36 (dd, 1H), 3.88 (dd, 2H), 3.13-2.97 (m, 4H), 2.93 (d, 1H), 2.90-2.83 (m, 1H), 2.79 (s, 3H), 2.72 (m, 2H), 2.49-2.38 (m, 2H), 2.21 (s, 3H). MS (ESI) m/z 889 (M+H)⁺.

### Example 149

### (7R,16R,21S)-19-chloro-1-(4-fluorophenyl)-10-({2-[4-(hydroxymethyl)phenyl]pyrimidin-4-yl}methoxy)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid

### Example 149A

### methyl 2-(4-(((tert-butyldimethylsilyl)oxy)methyl)phenyl)pyrimidine-4-carboxylate

Methyl 2-chloropyrimidine-4-carboxylate (8.3 g) and (4-(((tert-butyldimethylsilyl)oxy)methyl)phenyl)boronic acid (13.44 g) were suspended in previously degassed 1,4-dioxane (83 mL). Potassium carbonate (8.31 g) was solubilized in previously degassed water (83 mL) and added to the reaction mixture. 1,1′-Bis(diphenylphosphino)ferrocene-palladium(II)dichloride dichloromethane complex (1.178 g) was added and the reaction mixture was stirred at 80° C. for 4 hours under nitrogen gas. The reaction mixture was concentrated under reduced pressure and diluted with 100 mL of water and extracted with 3×100 mL of dichloromethane. The combined organic layers were dried over MgSO₄, filtered, and concentrated. Purification was performed by flash chromatography on a Biotage® silica gel cartridge (KPSil 340 g), eluting with 5-25% ethyl acetate in cyclohexane to afford the title compound. LC/MS (APCI) m/z 359.0 (M+H)⁺.

### Example 149B

### (2-(4-(((tert-butyldimethylsilyl)oxy)methyl)phenyl)pyrimidin-4-yl)methanol

To a solution of Example 149A (8.88 g) in tetrahydrofuran (53 mL) and methanol (106 mL) was added at −10° C., sodium borohydride (3.28 g). The reaction was stirred at 0° C. for 30 minutes. The reaction was quenched at 0° C. with 120 mL saturated aqueous NH₄Cl and the organic solvents were evaporated. The remaining mixture was diluted with 150 mL dichloromethane. The organic layer was collected and the aqueous phase was extracted with 2×75 mL dichloromethane, The organic layers were combined, dried with MgSO₄, filtered and concentrated. The crude material was purified on a silica gel column eluting with 5-20% ethyl acetate in cyclohexane to afford the title compound. LC/MS (APCI) m/z 331.0 (M+H)⁺.

### Example 149C

### tert-butyl (7R,16R,21S)-10-({2-[4-({[tert-butyl(dimethyl)silyl]oxy}methyl)phenyl]pyrimidin-4-yl}methoxy)-19-chloro-1-(4-fluorophenyl)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

A 4 mL vial, equipped with stir bar, was charged with Example 139E (60 mg), Example 149B (52.1 mg) and triphenylphosphine (43.4 mg). The vial was capped with septa and evacuated and backfilled with nitrogen gas twice. Toluene (0.79 mL) was added and once all the reagents completely dissolved, the mixture was cooled with an ice bath. Di-tert-butyl azodicarboxylate (36.3 mg) was added in one portion. The vial was capped with septa, evacuated and backfilled with nitrogen gas twice again. The mixture was stirred at 0° C. for 10 minutes and at ambient overnight. The mixture was concentrated and purified by silica gel flash chromatography on Analogix Intelliflash²⁸⁰ system (eluting 0-8% methanol/CH₂Cl₂) to afford the title compound. MS (ESI) m/z 1073.4 (M+H)⁺.

### Example 149D

### (7R,16R,21S)-19-chloro-1-(4-fluorophenyl)-10-({2-[4-(hydroxymethyl)phenyl]pyrimidin-4-yl}methoxy)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid

To a solution of Example 149C (66 mg) in CH₂Cl₂ (0.66 mL) was added trifluoroacetic acid (0.66 mL). The mixture was stirred for 5 hours and concentrated in vacuo overnight. The material was taken up in tetrahydrofuran (0.40 mL) and methanol (0.40 mL). To the mixture was added lithium hydroxide solution (1.0 M in H₂O, 0.49 mL) and the mixture was stirred for 10 minutes. Dimethylformamide was added and the solution was neutralized with trifluoroacetic acid. The reaction mixture was purified on a Gilson prep HPLC (Zorbax, C-18, 250×21.2 mm column, 5-75% acetonitrile in water (0.1% trifluoroacetic acid)) to give the title compound after lyophilization. ¹H NMR (400 MHz, dimethyl sulfoxide-d₆) δ ppm 9.52 (s, 1H), 8.90 (d, J=5.1 Hz, 1H), 8.75 (s, 1H), 8.40-8.31 (m, 2H), 7.51-7.44 (m, 3H), 7.25-7.08 (m, 5H), 6.97 (d, J=8.3 Hz, 1H), 6.92 (d, J=9.0 Hz, 1H), 6.83 (dd, J=9.0, 2.9 Hz, 1H), 6.17 (dd, J=5.2, 3.0 Hz, 1H), 5.68 (d, J=2.8 Hz, 1H), 5.32-5.14 (m, 2H), 4.63-4.54 (m, 3H), 4.47 (d, J=12.9 Hz, 1H), 4.36 (dd, J=13.2, 8.5 Hz, 1H), 3.89 (dd, J=17.0, 5.4 Hz, 1H), 3.38-2.82 (m, 9H), 2.78 (s, 3H), 2.73 (t, J=5.0 Hz, 2H), 2.22 (s, 3H). MS (ESI) m/z 903.4 (M+H)⁺.

### Example 150

### (7R,16R,21S)-19-chloro-1-(4-fluorophenyl)-10-{[2-(4-hydroxyphenyl)pyrimidin-4-yl]methoxy}-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid

### Example 150A

### tert-butyl (7R,16R,21S)-10-{[2-(4-{[tert-butyl(dimethyl)silyl]oxy}phenyl)pyrimidin-4-yl]methoxy}-19-chloro-1-(4-fluorophenyl)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

The title compound was prepared using the conditions described in Example 149C, substituting Example 125B for Example 149B. MS (ESI) m/z 1059.4 (M+H)⁺.

### Example 150B

### (7R,16R,21S)-10-{[2-(4-{[tert-butyl(dimethyl)silyl]oxy}phenyl)pyrimidin-4-yl]methoxy}-19-chloro-1-(4-fluorophenyl)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid

To a solution of Example 150A (60 mg) in CH₂Cl₂ (0.60 mL) was added trifluoroacetic acid (0.60 mL). The mixture was stirred for 5 hours, and concentrated in vacuo to give the title compound. MS (ESI) m/z 1003.7 (M+H)⁺.

### Example 150C

### (7R,16R,21S)-19-chloro-1-(4-fluorophenyl)-10-{[2-(4-hydroxyphenyl)pyrimidin-4-yl]methoxy}-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid

To a solution of Example 150B (57.2 mg) in CH₂Cl₂ (2 mL) was added tetrabutylammonium fluoride solution (1.0 M in tetrahydrofuran, 0.228 mL). The mixture was stirred for one day. Dimethylformamide was added to dissolve the material. The reaction mixture was purified on a Gilson prep HPLC (Zorbax, C-18, 250×21.2 mm column, 5-75% acetonitrile in water (0.1% trifluoroacetic acid)) to give the title compound after lyophilization. ¹H NMR (400 MHz, dimethyl sulfoxide-d₆) δ ppm 8.81 (d, J=5.1 Hz, 1H), 8.75 (s, 1H), 8.31-8.21 (m, 2H), 7.39 (d, J=5.1 Hz, 1H), 7.24-7.11 (m, 5H), 6.96 (d, J=8.3 Hz, 1H), 6.89 (d, J=8.9 Hz, 3H), 6.82 (dd, J=9.1, 2.9 Hz, 1H), 6.16 (dd, J=5.2, 3.0 Hz, 1H), 5.67 (d, J=2.8 Hz, 1H), 5.19 (q, J=15.1 Hz, 2H), 4.59 (q, J=6.4 Hz, 1H), 4.47 (d, J=12.9 Hz, 1H), 4.36 (dd, J=13.2, 8.5 Hz, 1H), 3.88 (dd, J=17.0, 5.4 Hz, 1H), 3.44-2.81 (m, 9H), 2.78 (s, 3H), 2.74 (d, J=4.4 Hz, 2H), 2.22 (s, 3H). MS (ESI) m/z 889.3 (M+H)⁺.

### Example 151

### (7R,16R,21S)-19-chloro-1-(4-fluorophenyl)-10-({2-[2-(hydroxymethyl)phenyl]pyrimidin-4-yl}methoxy)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid

### Example 151A

### ((2-bromobenzyl)oxy)(tert-butyl)dimethylsilane

To a solution of 2-bromobenzyl alcohol (5.00 g), imidazole (4.00 g) and tert-butyldimethylsilyl chloride (4.43 g) in dimethylformamide (18 mL) at 0° C. was added dropwise 4-dimethylaminopyridine (0.327 g) in dimethylformamide (2 mL). The reaction mixture was stirred for one day. The mixture was diluted with water and extracted with ether. The combined extracts were washed with saturated brine, dried over Na₂SO₄, filtered and concentrated in vacuo to afford the title compound. ¹H NMR (501 MHz, dimethyl sulfoxide-d₆) δ ppm 7.58 (dd, J=7.9, 1.2 Hz, 1H), 7.50 (ddt, J=7.7, 1.8, 0.9 Hz, 1H), 7.41 (td, J=7.5, 1.2 Hz, 1H), 7.22 (dddd, J=8.1, 7.3, 1.7, 0.9 Hz, 1H), 4.70 (d, J=0.9 Hz, 2H), 0.92 (s, 9H), 0.10 (s, 6H).

### Example 151B

### (2-(((tert-butyldimethylsilyl)oxy)methyl)phenyl)boronic acid

A 25 mL vial charged with potassium acetate (0.326 g) was dried in an 80° C. oven under vacuum for 16 hours and cooled under nitrogen gas. Tetrahydroxydiboron (0.298 g) and chloro[(tri-tert-butylphosphine)-2-(2-aminobiphenyl)] palladium(II) (0.043 g) were added and the mixture was evacuated under vacuum, refilled with nitrogen, and cooled to 0° C. A solution of Example 151A (0.50 g) in 30% ethylene glycol in methanol (4 mL) was transferred via cannula under nitrogen gas. The reaction mixture was stirred at 0° C. for 30 minutes and ambient temperature for one hour. The mixture was quenched with 20 mL brine and was transferred to a separatory funnel with 10 mL water and 30 mL ethyl acetate. The separated organic layer was washed with brine (20 mL), dried with MgSO₄, filtered and concentrated. The residue was purified by silica gel flash chromatography on Analogix Intelliflash²⁸⁰ system (eluting with 0-10% ethyl acetate/heptanes) to afford the title compound. ¹H NMR (501 MHz, dimethyl sulfoxide-d₆) δ ppm 7.55-7.48 (m, 1H), 7.38-7.29 (m, 2H), 7.20 (td, J=7.2, 1.5 Hz, 1H), 4.84 (s, 2H), 0.90 (s, 9H), 0.07 (s, 6H). LC-MS (ESI) m/z 267.1 (M+H)⁺.

### Example 151C

### (2-(2-(((tert-butyldimethylsilyl)oxy)methyl)phenyl)pyrimidin-4-yl)methanol

A stirring solution of (2-chloropyrimidin-4-yl)methanol (50 mg), Example 151B (101 mg) and tetrakis(triphenylphosphine)palladium(0) (40.0 mg) in tetrahydrofuran (2.2 mL) and saturated aqueous sodium bicarbonate solution (1.30 mL) was degassed by bubbling nitrogen gas through the mixture via syringe needle for 10 minutes. The mixture was stirred at 75° C. overnight. The mixture was diluted with water and was extracted with three portions of ethyl acetate. The combined organic layers were dried over anhydrous magnesium sulfate, filtered and concentrated. The residue was purified by silica gel flash chromatography on an AnaLogix IntelliFlash²⁸⁰ system (eluting with 0-40% ethyl acetate/hexanes) to give the title compound. MS (ESI) m/z 331.2 (M+H)⁺.

### Example 151D

### tert-butyl (7R,16R,21S)-10-({2-[2-({[tert-butyl(dimethyl)silyl]oxy}methyl)phenyl]pyrimidin-4-yl}methoxy)-19-chloro-1-(4-fluorophenyl)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylate

The title compound was prepared using the conditions described in Example 149C, substituting Example 151C for Example 149B. MS (ESI) m/z 1073.6 (M+H)⁺.

### Example 151E

### (7R,16R,21S)-19-chloro-1-(4-fluorophenyl)-10-({2-[2-(hydroxymethyl)phenyl]pyrimidin-4-yl}methoxy)-20-methyl-16-[(4-methylpiperazin-1-yl)methyl]-7,8,15,16-tetrahydro-18,21-etheno-13,9-(metheno)-6,14,17-trioxa-2-thia-3,5-diazacyclononadeca[1,2,3-cd]indene-7-carboxylic acid

To a solution of Example 151D (64 mg) in dichloromethane (0.66 mL) was added trifluoroacetic acid (0.66 mL). The mixture was stirred for 2 hours, concentrated in vacuo and dissolved in acetonitrile and N,N-dimethylformamide. The reaction mixture was purified on Gilson prep HPLC (Zorbax, C-18, 250×21.2 mm column, 5-75% acetonitrile in water (0.1% trifluoroacetic acid)) to give the title compound after lyophilization. ¹H NMR (400 MHz, dimethyl sulfoxide-d₆) δ ppm 9.51 (s, 1H), 8.95 (d, J=5.1 Hz, 1H), 8.75 (s, 1H), 7.97 (dd, J=7.7, 1.4 Hz, 1H), 7.69 (d, J=7.6 Hz, 1H), 7.59-7.48 (m, 2H), 7.42 (dd, J=8.1, 6.9 Hz, 1H), 7.25-7.12 (m, 5H), 6.94 (dd, J=19.1, 8.7 Hz, 2H), 6.84 (dd, J=9.0, 2.9 Hz, 1H), 6.17 (dd, J=5.2, 3.0 Hz, 1H), 5.68 (d, J=2.8 Hz, 1H), 5.23 (q, J=15.1 Hz, 2H), 4.82 (s, 2H), 4.60 (q, J=6.5 Hz, 1H), 4.47 (d, J=12.9 Hz, 1H), 4.36 (dd, J=13.2, 8.5 Hz, 1H), 3.88 (dd, J=17.1, 5.4 Hz, 1H), 3.43-2.84 (m, 9H), 2.79 (s, 3H), 2.74 (t, J=5.2 Hz, 2H), 2.22 (s, 3H). MS (ESI) m/z 903.4 (M+H)⁺.

### BIOLOGICAL EXAMPLES

### Exemplary MCL-1 Inhibitors Bind MCL-1

The ability of the exemplary MCL-1 inhibitors of Examples 1 through 151 to bind MCL-1 was demonstrated using the Time Resolved-Fluorescence Resonance Energy Transfer (TR-FRET) Assay. Tb-anti-GST antibody was purchased from Invitrogen (Catalog No. PV4216).

### Probe Synthesis

Reagents

All reagents were used as obtained from the vendor unless otherwise specified. Peptide synthesis reagents including diisopropylethylamine (DIEA), dichloromethane (DCM), N-methylpyrrolidone (NMP), 2-(1H-benzotriazole-1-yl)-1,1,3,3-tetramethyluronium hexafluorophosphate (HBTU), N-hydroxybenzotriazole (HOBt) and piperidine were obtained from Applied Biosystems, Inc. (ABI), Foster City, Calif. or American Bioanalytical, Natick, Mass.

Preloaded 9-Fluorenylmethyloxycarbonyl (Fmoc) amino acid cartridges (Fmoc-Ala-OH, Fmoc-Cys(Trt)-OH, Fmoc-Asp(tBu)-OH, Fmoc-Glu(tBu)-OH, Fmoc-Phe-OH, Fmoc-Gly-OH, Fmoc-His(Trt)-OH, Fmoc-Ile-OH, Fmoc-Leu-OH, Fmoc-Lys(Boc)-OH, Fmoc-Met-OH, Fmoc-Asn(Trt)-OH, Fmoc-Pro-OH, Fmor-Gln(Trt)-OH, Fmoc-Arg(Pbf)-OH, Fmoc-Ser(tBu)-OH, Fmoc-Thr(tBu)-OH, Fmoc-Val-OH, Fmoc-Trp(Boc)-OH, Fmoc-Tyr(tBu)-OH) were obtained from ABI or Anaspec, San Jose, Calif.

The peptide synthesis resin (Fmoc-Rink amide MBHA resin) and Fmoc-Lys(Mtt)-OH were obtained from Novabiochem, San Diego, Calif.

Single-isomer 6-carboxyfluorescein succinimidyl ester (6-FAM-NHS) was obtained from Anaspec.

Trifluoroacetic acid (TFA) was obtained from Oakwood Products, West Columbia, S.C.

Thioanisole, phenol, triisopropylsilane (TIS), 3,6-dioxa-1,8-octanedithiol (DODT) and isopropanol were obtained from Aldrich Chemical Co., Milwaukee, Wis.

Matrix-assisted laser desorption ionization mass-spectra (MALDI-MS) were recorded on an Applied Biosystems Voyager DE-PRO MS).

Electrospray mass-spectra (ESI-MS) were recorded on Finnigan SSQ7000 (Finnigan Corp., San Jose, Calif.) in both positive and negative ion mode.

General Procedure for Solid-Phase Peptide Synthesis (SPPS)

Peptides were synthesized with, at most, 250 μmol preloaded Wang resin/vessel on an ABI 433A peptide synthesizer using 250 μmol scale Fastmoc™ coupling cycles. Preloaded cartridges containing 1 mmol standard Fmoc-amino acids, except for the position of attachment of the fluorophore, where 1 mmol Fmoc-Lys(Mtt)-OH was placed in the cartridge, were used with conductivity feedback monitoring. N-terminal acetylation was accomplished by using 1 mmol acetic acid in a cartridge under standard coupling conditions.

Removal of 4-Methyltrityl (Mtt) from Lysine

The resin from the synthesizer was washed thrice with dichloromethane and kept wet. 150 mL of 95:4:1 dichloromethane:triisopropylsilane:trifluoroacetic acid was flowed through the resin bed over 30 minutes. The mixture turned deep yellow then faded to pale yellow. 100 mL of N,N-dimethylformamide (DMF) was flowed through the bed over 15 minutes. The resin was then washed thrice with DMF and filtered. Ninhydrin tests showed a strong signal for primary amine.

Resin Labeling with 6-Carboxyfluorescein-NHS (6-FAM-NHS)

The resin was treated with 2 equivalents 6-FAM-NHS in 1% DIEA/DMF and stirred or shaken at ambient temperature overnight. When complete, the resin was drained, washed thrice with DMF, thrice with (1×dichloromethane and 1×methanol) and dried to provide an orange resin that was negative by ninhydrin test.

General Procedure for Cleavage and Deprotection of Resin-Bound Peptide

Peptides were cleaved from the resin by shaking for 3 hours at ambient temperature in a cleavage cocktail consisting of 80% TFA, 5% water, 5% thioanisole, 5% phenol, 2.5% TIS, and 2.5% EDT (1 mL/0.1 g resin). The resin was removed by filtration and rinsing twice with TFA. The TFA was evaporated from the filtrates, and product was precipitated with ether (10 mL/0.1 g resin), recovered by centrifugation, washed twice with ether (10 mL/0.1 g resin) and dried to give the crude peptide.

General Procedure for Purification of Peptides

The crude peptides were purified on a Gilson preparative HPLC system running Unipoint® analysis software (Gilson, Inc., Middleton, Wis.) on a radial compression column containing two 25×100 mm segments packed with Delta-Pak™ C18 15 M particles with 100 A pore size and eluted with one of the gradient methods listed below. One to two milliliters of crude peptide solution (10 mg/mL in 90% DMSO/water) was purified per injection. The peaks containing the product(s) from each run were pooled and lyophilized. All preparative runs were run at 20 mL/minute with eluents as buffer A: 0.1% TFA-water and buffer B: acetonitrile.

General Procedure for Analytical HPLC

Analytical HPLC was performed on a Hewlett-Packard 1200 series system with a diode-array detector and a Hewlett-Packard 1046A fluorescence detector running HPLC 3D ChemStation software version A.03.04 (Hewlett-Packard. Palo Alto, Calif.) on a 4.6×250 mm YMC column packed with ODS-AQ 5 μm particles with a 120 Å pore size and eluted with one of the gradient methods listed below after preequilibrating at the starting conditions for 7 minutes. Eluents were buffer A: 0.1% TFA-water and buffer B: acetonitrile. The flow rate for all gradients was 1 mL/min.

Synthesis of Probe F-Bak

Peptide probe F-bak, which binds MCL-1, was synthesized as described below. Probe F-Bak is acetylated at the N-terminus, amidated at the C-terminus and has the amino acid sequence GQVGRQLAIIGDKINR (SEQ ID NO:1). It is fluoresceinated at the lysine residue (K) with 6-FAM. Probe F-Bak can be abbreviated as follows: acetyl-GQVGRQLAIIGDK(6-FAM)INR-NH₂.

To make probe F-Bak, Fmoc-Rink amide MBHA resin was extended using the general peptide synthesis procedure to provide the protected resin-bound peptide (1.020 g). The Mtt group was removed, labeled with 6-FAM-NHS and cleaved and deprotected as described hereinabove to provide the crude product (0.37 g). This product was purified by RP-HPLC. Fractions across the main peak were tested by analytical RP-HPLC, and the pure fractions were isolated and lyophilized, with the major peak providing the title compound (0.0802 g). MALDI-MS m/z=2137.1 [(M+H)⁺].

Alternative Synthesis of Peptide Probe F-Bak

In an alternative method, the protected peptide was assembled on 0.25 mmol Fmoc-Rink amide MBHA resin (Novabiochem) on an Applied Biosystems 433A automated peptide synthesizer running Fastmoc™ coupling cycles using pre-loaded 1 mmol amino acid cartridges, except for the fluorescein(6-FAM)-labeled lysine, where 1 mmol Fmoc-Lys(4-methyltrityl) was weighed into the cartridge. The N-terminal acetyl group was incorporated by putting 1 mmol acetic acid in a cartridge and coupling as described hereinabove. Selective removal of the 4-methyltrityl group was accomplished with a solution of 95:4:1 DCM:TIS:TFA (v/v/v) flowed through the resin over 15 minutes, followed by quenching with a flow of dimethylformamide. Single-isomer 6-carboxyfluorescein-NHS was reacted with the lysine side-chain in 1% DIEA in DMF and confirmed complete by ninhydrin testing. The peptide was cleaved from the resin and side-chains deprotected by treating with 80:5:5:5:2.5:2.5 TFA/water/phenol/thioanisole/triisopropylsilane: 3,6-dioxa-1,8-octanedithiol (v/v/v/v/v/v), and the crude peptide was recovered by precipitation with diethyl ether. The crude peptide was purified by reverse-phase high-performance liquid chromatography, and its purity and identity were confirmed by analytical reverse-phase high-performance liquid chromatography and matrix-assisted laser-desorption mass-spectrometry (m/z=2137.1 ((M+H)⁺).

Time Resolved-Fluorescence Resonance Energy Transfer (TR-FRET) Assay

The ability of exemplary MCL-1 inhibitors Example 1 to Example 151 to compete with probe F-Bak for binding MCL-1 was demonstrated using a Time Resolved Fluorescence Resonance Energy Transfer (TR-FRET) binding assay.

Method

For the assay, test compounds were serially diluted in DMSO starting at 50 μM (2× starting concentration; 10% DMSO) and 10 μL transferred into a 384-well plate. 10 μL of a protein/probe/antibody mix was then added to each well at final concentrations listed below:

| | | |
|---|---|---|
| Protein: | GST-MCL-1 | 1 nM |
| Antibody | Tb-anti-GST | 1 nM |
| Probe: | F-Bak | 100 nM |

The samples were then mixed on a shaker for 1 minute and incubated for an additional 2 hours at room temperature. For each assay plate, a probe/antibody and protein/antibody/probe mixture were included as a negative and a positive control, respectively. Fluorescence was measured on the Envision (Perkin Elmer) using a 340/35 nm excitation filter and 520/525 (F-Bak) and 495/510 nm (Tb-labeled anti-his antibody) emission filters. Dissociation constants (K₁) were determined using Wang's equation (Wang, 1995, FEBS Lett. 360:111-114). The TR-FRET assay can be performed in the presence of varying concentrations of human serum (HS) or fetal bovine serum (FBS). Compounds were tested both without HS and in the presence of 10% HS.

Results

The results of binding assays (Kᵢ in nanomolar) are provided in Table 2, below, and demonstrate the ability of compounds of the disclosure to bind MCL-1 protein.

TABLE 2
TR-FRET MCL-1 Binding Data

| | MCL-1 Binding | MCL-1 Binding |
|---|---|---|
| Example | Kᵢ (nM) | Kᵢ (nM, 10% HS) |
| 1 | 0.066 | 0.520 |
| 2 | 2.890 | 18.437 |
| 3 | 0.114 | 0.878 |
| 4 | 0.299 | 1.677 |
| 5 | 1.234 | 10.162 |
| 6 | 0.855 | 10.174 |
| 7 | 142.211 | >444 |
| 8 | 1.156 | 4.676 |
| 9 | 56.478 | 205.000 |
| 10 | 0.157 | 1.945 |
| 11 | 0.042 | 0.242 |
| 12 | 18.148 | 52.930 |
| 13 | 46.144 | 397.339 |
| 14 | 0.334 | 73.087 |
| 15 | 45.920 | 402.000 |
| 16 | 0.169 | 0.892 |
| 17 | 0.620 | 23.007 |
| 18 | 0.708 | 170.118 |
| 19 | 9.655 | 157.000 |
| 20 | 0.106 | 0.959 |
| 21 | 9.987 | 36.942 |
| 22 | 0.123 | 3.075 |
| 23 | 0.364 | 6.401 |
| 24 | 0.181 | 4.634 |
| 25 | 0.182 | 0.893 |
| 26 | 19.100 | 58.300 |
| 27 | 0.563 | 1.286 |
| 28 | 0.626 | 1.296 |
| 29 | NT | NT |
| 30 | 0.377 | 4.625 |
| 31 | 0.156 | 1.165 |
| 32 | 0.074 | 0.404 |
| 33 | 37.506 | 122.833 |
| 34 | 0.056 | 0.350 |
| 35 | 0.154 | 1.553 |
| 36 | 5.815 | 86.744 |
| 37 | 0.067 | 0.204 |
| 38 | 0.322 | 3.353 |
| 39 | 0.187 | 3.029 |
| 40 | 0.083 | 0.735 |
| 41 | 0.135 | 1.156 |
| 42 | 0.070 | 0.395 |
| 43 | 0.178 | 2.541 |
| 44 | NT | NT |
| 45 | NT | NT |
| 46 | 0.108 | 0.300 |
| 47 | 0.978 | 10.000 |
| 48 | 0.231 | 1.170 |
| 49 | 0.651 | 6.672 |
| 50 | 0.104 | 0.819 |
| 51 | 0.239 | 4.045 |
| 52 | 0.176 | 1.079 |
| 53 | 5.404 | 197.221 |
| 54 | 0.090 | 0.846 |
| 55 | 0.070 | 0.721 |
| 56 | NT | NT |
| 57 | 0.171 | 0.845 |
| 58 | 0.059 | 0.896 |
| 59 | 11.645 | 50.993 |
| 60 | 2.460 | 12.908 |
| 61 | 0.047 | 1.538 |
| 62 | 0.056 | 0.451 |
| 63 | 0.933 | 30.209 |
| 64 | 0.456 | 18.676 |
| 65 | 0.057 | 0.943 |
| 66 | 0.060 | 0.413 |
| 67 | NT | NT |
| 68 | 0.064 | 2.019 |
| 69 | 3.473 | 27.710 |
| 70 | 4.432 | 46.164 |
| 71 | 0.290 | 4.150 |
| 72 | 34.000 | 296.000 |
| 73 | 0.029 | 0.140 |
| 74 | 0.102 | 0.317 |
| 75 | 0.096 | 3.428 |
| 76 | 0.127 | 1.390 |
| 77 | NT | NT |
| 78 | 0.139 | 0.499 |
| 79 | 0.108 | 13.050 |
| 80 | 7.750 | 259.000 |
| 81 | 0.093 | 7.620 |
| 82 | 0.012 | 0.162 |
| 83 | 0.703 | 3.940 |
| 84 | 1.600 | 32.300 |
| 85 | 0.216 | 1.130 |
| 86 | 0.136 | 0.807 |
| 87 | 0.481 | 1.880 |
| 88 | 0.017 | 2.700 |
| 89 | 3.390 | 22.300 |
| 90 | 11.900 | 73.300 |
| 91 | 0.027 | 0.616 |
| 92 | 0.006 | 0.172 |
| 93 | 0.029 | 0.138 |
| 94 | 0.012 | 0.291 |
| 95 | 0.011 | 0.278 |
| 96 | 2.315 | 15.530 |
| 97 | 5.659 | 31.812 |
| 98 | 1.106 | 10.599 |
| 99 | 1.812 | 9.042 |
| 100 | 0.336 | 2.651 |
| 101 | 0.040 | 0.560 |
| 102 | 0.009 | 0.581 |
| 103 | 0.020 | 0.609 |
| 104 | 0.254 | 1.304 |
| 105 | 0.121 | 2.287 |
| 106 | 20.469 | 244.753 |
| 107 | 13.880 | 44.074 |
| 108 | 0.042 | 12.996 |
| 109 | 13.409 | 122.248 |
| 110 | 1.362 | 11.283 |
| 111 | 0.129 | 12.749 |
| 112 | 0.828 | 310.000 |
| 113 | 0.029 | 7.756 |
| 114 | 0.030 | 0.665 |
| 115 | 0.016 | 0.203 |
| 116 | 0.007 | 0.236 |
| 117 | 0.483 | 6.919 |
| 118 | 0.043 | 0.522 |
| 119 | 0.027 | 1.785 |
| 120 | 4.411 | 108.314 |
| 121 | 0.339 | 13.100 |
| 122 | 0.027 | 2.734 |
| 123 | 0.177 | 1.876 |
| 124 | 0.045 | 0.596 |
| 125 | 0.164 | 1.408 |
| 126 | 1.749 | 12.332 |
| 127 | 3.32 | 160 |
| 128 | 0.353 | 13.4 |
| 129 | 0.213 | 4.77 |
| 130 | 0.474 | 24.5 |
| 131 | 0.395 | 1.415 |
| 132 | 0.259 | 1.361 |
| 133 | 0.009 | 0.707 |
| 134 | 0.187 | 2.15 |
| 135 | 0.314 | 6.28 |
| 136 | 9.09 | 198 |
| 137 | 100.011 | 284.268 |
| 138 | 51.323 | 52.225 |
| 139 | 0.025 | 0.334 |
| 140 | 0.026 | 0.282 |
| 141 | 0.089 | 0.716 |
| 142 | 0.08 | 0.44 |
| 143 | 56.717 | 45.92 |
| 144 | 4.287 | 0.274 |
| 145 | 0.047 | 0.237 |
| 146 | 0.762 | 0.247 |
| 147 | 0.037 | 0.11 |
| 148 | 0.261 | 3.903 |
| 149 | 0.032 | 0.281 |
| 150 | 0.033 | 0.228 |
| 151 | 0.024 | 0.089 |

NT = not tested, NV = not valid

### Exemplary MCL-1 Inhibitors Demonstrate In Vitro Efficacy in Tumor Cell Viability Assays

The in vitro efficacy of exemplary MCL-1 inhibitors can be determined in cell-based killing assays using a variety of cell lines and mouse tumor models. For example, their activity on cell viability can be assessed on a panel of cultured tumorigenic and non-tumorigenic cell lines, as well as primary mouse or human cell populations. MCL-1 inhibitory activity of exemplary MCL-1 inhibitors was confirmed in a cell viability assay with AMO-1 and NCI-H929 human multiple myeloma tumor cell lines.

Method

In one exemplary set of conditions, NCI-H929 or AMO-1 (ATCC, Manassas, Va.) were plated 4,000 cells per well in 384-well tissue culture plates (Corning, Corning, NY) in a total volume of 25 μL RPMI tissue culture medium supplemented with 10% fetal bovine serum (Sigma-Aldrich, St. Louis, Mo.) and treated with a 3-fold serial dilution of the compounds of interest with a Labcyte Echo from a final concentration of 10 μM to 0.0005 μM. Each concentration was tested in duplicate at least 3 independent times. A luminescent signal proportional to the number of viable cells following 24 hours of compound treatment was determined using the CellTiter-Glo® Luminescent Cell Viability Assay according to the manufacturer's recommendations (Promega Corp., Madison, Wis.). The plates were read in a Perkin Elmer Envision using a Luminescence protocol. To generate dose response curves the data is normalized to percent viability by setting the averages of the staurosporine (10 μM) and DMSO only control wells to 0% and 100% viability respectively. The IC50 values for the compounds are generated by fitting the normalized data with Accelrys Assay Explorer 3.3 to a sigmoidal curve model using linear regression, Y=(100*xn)/(Kn+xn), where Y is the measured response, x is the compound concentration, n is the Hill Slope and K is the IC50 and the lower and higher asymptotes are constrained to 0 and 100 respectively. Results

The results of AMO-1 and H929 cell viability assays (IC₅₀ in nanomolar) carried out in the presence of 10% FBS for exemplary MCL-1 inhibitors are provided in Table 3, below. The results demonstrate the ability of compounds of the disclosure to potently inhibit the growth of human tumor cells in vitro.

TABLE 3
MCL-1 Inhibitor In Vitro Cell Efficacy Data

| | AMO-1 Viability | H929 Viability |
|---|---|---|
| | IC₅₀ (μM, 10% | IC₅₀ (μM, |
| EXAMPLE | FBS) | 10% FBS) |
| 1 | 0.2236 | 0.1486 |
| 2 | NT | 4.5999 |
| 3 | NT | 0.5850 |
| 4 | NT | 1.0085 |
| 5 | NT | 1.0604 |
| 6 | NT | 1.6371 |
| 7 | NT | NT |
| 8 | NT | 3.0028 |
| 9 | NT | >10.00 |
| 10 | NT | >10.00 |
| 11 | NT | 1.1576 |
| 12 | NT | >10.00 |
| 13 | NT | 0.7300 |
| 14 | NT | >10.00 |
| 15 | NT | >10.00 |
| 16 | NT | 4.2177 |
| 17 | NT | 2.7774 |
| 18 | NT | 3.6400 |
| 19 | NT | >10.00 |
| 20 | NT | 0.2339 |
| 21 | NT | 0.0130 |
| 22 | NT | 0.3086 |
| 23 | NT | 1.0492 |
| 24 | NT | 0.4263 |
| 25 | NT | 3.3195 |
| 26 | NT | >10.00 |
| 27 | NT | 0.1133 |
| 28 | NT | 0.0877 |
| 29 | NT | 0.2857 |
| 30 | NT | 0.7781 |
| 31 | NT | 0.2596 |
| 32 | NT | 0.1761 |
| 33 | NT | 3.6575 |
| 34 | NT | 0.9134 |
| 35 | NT | 2.0319 |
| 36 | NT | >10.00 |
| 37 | NT | 0.4014 |
| 38 | NT | 1.0040 |
| 39 | NT | 0.5261 |
| 40 | NT | 0.6459 |
| 41 | NT | 0.9276 |
| 42 | NT | 0.1196 |
| 43 | NT | 0.6690 |
| 44 | NT | NT |
| 45 | NT | 1.5997 |
| 46 | NT | 0.6949 |
| 47 | NT | 9.7800 |
| 48 | NT | 0.3178 |
| 49 | 0.0132 | 0.0091 |
| 50 | NT | 0.3358 |
| 51 | NT | 3.2133 |
| 52 | NT | 2.7323 |
| 53 | NT | >10.00 |
| 54 | NT | >10.00 |
| 55 | NT | >10.00 |
| 56 | NT | 1.0200 |
| 57 | NT | 0.1666 |
| 58 | 0.2678 | 0.1382 |
| 59 | NT | 5.3770 |
| 60 | 0.0693 | 0.1234 |
| 61 | 0.1800 | 0.2317 |
| 62 | 1.1800 | 1.8500 |
| 63 | 0.0723 | 0.1602 |
| 64 | NT | 0.1248 |
| 65 | 0.2844 | 0.1294 |
| 66 | 0.1570 | 0.0961 |
| 67 | NT | NT |
| 68 | 0.0189 | 0.0441 |
| 69 | 0.8384 | 2.2689 |
| 70 | 1.2507 | 4.6048 |
| 71 | 0.2273 | 0.4209 |
| 72 | >10.00 | >10.00 |
| 73 | 0.0008 | 0.0016 |
| 74 | 0.0474 | 0.0840 |
| 75 | 0.0016 | 0.0044 |
| 76 | 0.0873 | 0.1906 |
| 77 | NT | NT |
| 78 | 1.0210 | 0.3384 |
| 79 | 0.2540 | 0.7060 |
| 80 | >10.00 | >10.00 |
| 81 | 0.2736 | 0.3906 |
| 82 | 0.0355 | 0.0500 |
| 83 | >1.0 | >1.0 |
| 84 | >1.0 | >1.0 |
| 85 | >1.0 | >1.0 |
| 86 | 0.7220 | 0.6773 |
| 87 | >1.0 | >1.0 |
| 88 | 0.0029 | 0.0085 |
| 89 | >1.0 | >1.0 |
| 90 | >1.0 | >1.0 |
| 91 | 0.0196 | 0.0380 |
| 92 | 0.1836 | 0.1984 |
| 93 | 0.1336 | 0.2267 |
| 94 | 0.4437 | 0.3698 |
| 95 | 0.3348 | 0.2432 |
| 96 | >1.0 | >1.0 |
| 97 | >1.0 | >1.0 |
| 98 | 0.5590 | >1.0 |
| 99 | >1.0 | >1.0 |
| 100 | >1.0 | 0.6230 |
| 101 | >1.0 | >1.0 |
| 102 | 0.3719 | 0.2796 |
| 103 | 0.0063 | 0.0089 |
| 104 | 0.0052 | 0.0139 |
| 105 | 0.0028 | 0.0103 |
| 106 | 0.4026 | 0.8143 |
| 107 | 0.2121 | 0.7546 |
| 108 | 0.0011 | 0.0031 |
| 109 | 0.1635 | 0.2839 |
| 110 | 0.0316 | 0.0719 |
| 111 | 0.0135 | 0.0390 |
| 112 | 0.0510 | 0.1660 |
| 113 | 0.1469 | 0.1820 |
| 114 | 0.0658 | 0.0679 |
| 115 | 0.0101 | 0.0146 |
| 116 | 0.0002 | 0.0005 |
| 117 | 0.0113 | 0.0209 |
| 118 | 0.0902 | 0.1479 |
| 119 | 0.2212 | 0.1414 |
| 120 | 0.1037 | 0.2413 |
| 121 | 0.0120 | 0.0517 |
| 122 | 0.0013 | 0.0043 |
| 123 | 0.5631 | 0.5333 |
| 124 | 0.2955 | 0.2421 |
| 125 | >1.0 | 0.7403 |
| 126 | 0.5299 | >1.0 |
| 127 | 0.3710 | 0.8120 |
| 128 | 0.1579 | 0.3052 |
| 129 | 0.0198 | 0.0681 |
| 130 | >1.0 | >1.0 |
| 131 | 0.8560 | 0.6243 |
| 132 | 0.6420 | 0.5328 |
| 133 | 0.0021 | 0.0033 |
| 134 | 0.0117 | 0.0217 |
| 135 | 0.0375 | 0.0551 |
| 136 | 0.0139 | 0.1164 |
| 137 | 0.6690 | >1.0 |
| 138 | 0.0507 | 0.1170 |
| 139 | 0.0003 | 0.0025 |
| 140 | 0.0003 | 0.0010 |
| 141 | >1.0 | 0.6458 |
| 142 | 0.3456 | 0.3189 |
| 143 | >1.0 | >1.0 |
| 144 | 0.0010 | 0.0035 |
| 145 | 0.0028 | 0.0070 |
| 146 | 0.0052 | 0.0149 |
| 147 | 0.0006 | 0.0014 |
| 148 | 0.0011 | 0.0056 |
| 149 | 0.0010 | 0.0024 |
| 150 | 0.0033 | 0.0069 |
| 151 | 0.0011 | 0.0020 |

NT = not tested, NV = not valid

The ability of certain exemplary compounds of the present disclosure to inhibit the growth of tumor cells in mice was demonstrated in xenograft models derived from a human multiple myeloma cell line, AMO-1.

### Evaluation of Efficacy in Xenograft Models Methods

AMO-1 cells were obtained from the Deutsche Sammlung von Microorganismen und Zellkulturen (DSMZ, Braunschweig, Germany). The cells were cultured as monolayers in RPMI-1640 culture media (Invitrogen, Carlsbad, Calif.) that was supplemented with 10% Fetal Bovine Serum (FBS, Hyclone, Logan, Utah). To generate xenografts, 5×10⁶ viable cells were inoculated subcutaneously into the right flank of immune deficient female SCID/bg mice (Charles River Laboratories, Wilmington, Mass.) respectively. The injection volume was 0.2 mL and composed of a 1:1 mixture of S MEM and Matrigel (BD, Franklin Lakes, N.J.). Tumors were size matched at approximately 200 mm³. MCL-1 inhibitors were Formulated in 5% DMSO, 20% cremaphor EL and 75% D5W for injection and injected intraperitoneally. Injection volume did not exceed 200 μL. Alternatively, MCL-1 inhibitors were Formulated in 5% DMSO, 10% cremaphor and 85% D5W for injection and injected intravenously. Injection volume did not exceed 200 μL. Therapy began within 24 hours after size matching of the tumors. Mice weighed approximately 21 g at the onset of therapy. Tumor volume was estimated two to three times weekly. Measurements of the length (L) and width (W) of the tumor were taken via electronic caliper and the volume was calculated according to the following equation: V=L×W²/2. Mice were euthanized when tumor volume reached 3,000 mm³ or skin ulcerations occurred. Eight mice were housed per cage. Food and water were available ad libitum. Mice were acclimated to the animal facilities for a period of at least one week prior to commencement of experiments. Animals were tested in the light phase of a 12-hour light: 12-hour dark schedule (lights on at 06:00 hours).

To refer to efficacy of therapeutic agents, parameters of amplitude (TGIₘₐₓ), and durability (TGD) of therapeutic response are used. TGIₘₐₓ is the maximum tumor growth inhibition during the experiment. Tumor growth inhibition is calculated by 100*(1−Tᵥ/Cᵥ) where Tᵥ and Cᵥ are the mean tumor volumes of the treated and control groups, respectively. TGD or tumor growth delay is the extended time of a treated tumor needed to reach a volume of 1 cm³ relative to the control group. TGD is calculated by 100*(Tₜ/Cₜ−1) where Tₜ and Cₜ are the median time periods to reach 1 cm³ of the treated and control groups, respectively.

Results

As shown in Tables 4-8, compounds of the present disclosure are efficacious in an AMO-1 xenograft model of multiple myeloma, rendering significant tumor growth inhibition and tumor growth delay after intraperitoneal (IP) dosing of drug.

TABLE 4
In vivo efficacy of MCL-1 inhibitors in AMO-1 Xenograft Model

| | Dose | Route/ | TGIₘₐₓ | TGD |
|---|---|---|---|---|
| Treatment | (mg/kg/day) | Regimen | (%) | (%) |
| Vehicle | 0 | IP^{(a)}/QD × 1 | 0 | 0 |
| Example 1 | 100 | IP^{(a)}/QD × 5 | 56* | 46* |

^{(a)}IP Formulation = 5% DMSO, 20% cremophor EL, 75% D5W
*= p < 0.05 as compared to control treatment
8 mice per treatment group

TABLE 5
In vivo efficacy of MCL-1 inhibitors in AMO-1 Xenograft Model

| | Dose | Route/ | TGIₘₐₓ | |
|---|---|---|---|---|
| Treatment | (mg/kg/day) | Regimen | (%) | TGD (%) |
| Vehicle | 0 | IP^{(a)}/QD × 1 | 0 | 0 |
| Example 68 | 100 | IP/QD × 1 | 71* | 36* |
| Example 68 | 100 | IP/QD × 5 | 99* | 343* |

^{(a)}IP Formulation = 5% DMSO, 20% cremophor EL, 75% D5W
*= p < 0.05 as compared to control treatment
8 mice per treatment group

TABLE 6
In vivo efficacy of MCL-1 inhibitors in AMO-1 Xenograft Model

| | Dose | Route/ | TGIₘₐₓ | TGD |
|---|---|---|---|---|
| Treatment | (mg/kg/day) | Regimen | (%) | (%) |
| Vehicle | 0 | IP^{(a)}/QD × 1 | 0 | 0 |
| Example 63 | 100 | IP/QD × 1 | 19* | 0 |
| Example 49 | 100 | IP/QD × 1 | 87* | 139* |

^{(a)}IP Formulation = 5% DMSO, 20% cremophor EL, 75% D5W
*= p < 0.05 as compared to control treatment
8 mice per treatment group

TABLE 7
In vivo efficacy of MCL-1 inhibitors in AMO-1 Xenograft Model

| | Dose | Route/ | TGIₘₐₓ | TGD |
|---|---|---|---|---|
| Treatment | (mg/kg/day) | Regimen | (%) | (%) |
| Vehicle | 0 | IP^{(a)}/QD × 1 | 0 | 0 |
| Example 73 | 25 | IP/QD × 1 | 99* | 235* |

^{(a)}IP Formulation = 5% DMSO, 20% cremophor EL, 75% D5W
*= p < 0.05 as compared to control treatment
8 mice per treatment group

TABLE 8
In vivo efficacy of MCL-1 inhibitors in AMO-1 Xenograft Model

| | Dose | Route/ | TGIₘₐₓ | TGD |
|---|---|---|---|---|
| Treatment | (mg/kg/day) | Regimen | (%) | (%) |
| Vehicle | 0 | IP^{(a)}/QD × 1 | 0 | 0 |
| Example 73 | 25 | IP/QD × 1 | 97* | >92* |
| Example 88 | 25 | IP/QD × 1 | 84* | 58* |
| Example 112 | 25 | IP/QD × 1 | 61* | 17* |
| Example 75 | 25 | IP/QD × 1 | 76* | 75* |
| Example 108 | 25 | IP/QD × 1 | 70* | 33* |
| Example | 25 | IP/QD × 1 | 79* | 58* |
| 122^{(b)} | | | | |

^{(a)}IP Formulation = 5% DMSO, 20% cremophor EL, 75% D5W
*= p < 0.05 as compared to control treatment
7 mice per treatment group, 6 per group in^{(b)}

It is understood that the foregoing detailed description and accompanying examples are merely illustrative and are not to be taken as limitations upon the scope of the disclosure, which is defined solely by the appended claims and their equivalents. Various changes and modifications to the disclosed embodiments will be apparent to those skilled in the art. All publications, patents, and patent applications cited herein are hereby incorporated by reference in their entirety for all purposes.

We claim:

1. A compound of Formula (I) or a pharmaceutically acceptable salt thereof, 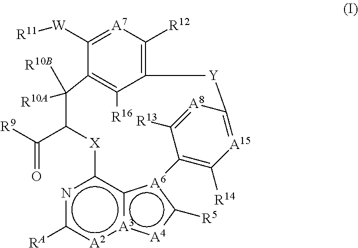
wherein
A² is CR², A³ is N, A⁴ is CR^{4a}, and A⁶ is C; or
A² is CR², A³ is N, A⁴ is O or S, and A⁶ is C; or
A² is N, A³ is C, A⁴ is O or S and A⁶ is C; or
A² is N, A³ is C, A⁴ is CR^{4a}, and A⁶ is N;
R^{A} is hydrogen, CH₃, halogen, CN, CH₂F, CHF₂, or CF₃;
X is O, or N(R^{x2}); wherein R^{x2} is hydrogen, C₁-C₃ alkyl, or unsubstituted cyclopropyl;
Y is (CH₂)ₘ, —CH═CH—(CH₂)ₙ—, —(CH₂)ₚ—CH═CH—, or —(CH₂)_{q}—CH═CH—(CH₂)ᵣ—; wherein 0, 1, 2, or 3 CH₂ groups are each independently replaced by O, N(R^{ya}), C(R^{ya})(R^{yb}), C(O), NC(O)R^{ya}, or S(O)₂;
m is 2, 3, 4, or 5;
n is 1, 2, or 3;
p is 1, 2, or 3;
q is 1 or 2; and
r is 1 or 2; wherein the sum of q and r is 2 or 3;
R^{ya}, at each occurrence, is independently hydrogen, C₂-C₆ alkenyl, C₂-C₆ alkynyl, G¹, C₁-C₆ alkyl, or C₁-C₆ haloalkyl; wherein the C₂-C₆ alkenyl, C₂-C₆ alkynyl, C₁-C₆ alkyl, and C₁-C₆ haloalkyl are optionally substituted with 1 or 2 substituents independently selected from the group consisting of oxo, —N(R^{yd})(R^{ye}), G¹, —OR^{yf}, —SR^{yg}, —S(O)₂N(R^{yd})(R^{ye}), and —S(O)₂-G¹; and
R^{yb} is C₂-C₆ alkenyl, C₂-C₆ alkynyl, C₁-C₆ alkyl, or C₁-C₆ haloalkyl; wherein the C₂-C₆ alkenyl, C₂-C₆ alkynyl, C₁-C₆ alkyl, and C₁-C₆ haloalkyl are optionally substituted with 1 or 2 substituents independently selected from the group consisting of oxo, —N(R^{yd})(R^{ye}), G¹, —OR^{yf}, —SR^{yg}, —S(O)₂N(R^{yd})(R^{ye}), and —S(O)₂-G¹; or
R^{ya} and R^{yb} together with the carbon atom to which they are attached, form a C₃-C₇ monocyclic cycloalkyl, C₄-C₇ monocyclic cycloalkenyl, or a 4-7 membered monocyclic heterocycle; wherein the C₃-C₇ monocyclic cycloalkyl, C₄-C₇ monocyclic cycloalkenyl, and the 4-7 membered monocyclic heterocycle are each optionally substituted with 1 —OR^{m} and 0, 1, 2, or 3 independently selected R^{s} groups;
R^{yd}, R^{ye}, R^{yf}, and R^{yg}, at each occurrence, are each independently hydrogen, G¹, C₁-C₆ alkyl, or C₁-C₆ haloalkyl; wherein the C₁-C₆ alkyl and the C₁-C₆ haloalkyl are optionally substituted with one substituent selected from the group consisting of G¹, —OR^{yh}, —SR^{yh}, —SO₂R^{yh}, and —N(R^{yi})(R^{yk});
G¹, at each occurrence, is piperazinyl, piperidinyl, pyrrolidinyl, thiomorpholinyl, tetrahydropyranyl, morpholinyl, or oxetanyl; wherein each G¹ is optionally substituted with 1 —OR^{m} and 0, 1, 2, or 3 substituents independently selected from the group consisting of G², —(C₁-C₆ alkylenyl)-G², and R^{s};
G², at each occurrence, is a C₃-C₇ monocyclic cycloalkyl, C₄-C₇ monocyclic cycloalkenyl, oxetanyl, or morpholinyl; wherein each G² is optionally substituted with 1 independently selected R^{t} groups;
R² is independently hydrogen, halogen, CH₃, or CN;
R^{4a}, at each occurrence, is independently hydrogen, halogen, CN, C₂-C₄ alkenyl, C₂-C₄ alkynyl, C₁-C₄ alkyl, C₁-C₄ haloalkyl, G^{A}, C₁-C₄ alkyl-G^{A}, or C₁-C₄alkyl-O-G^{A}; wherein each G^{A} is independently C₆-C₁₀ aryl, C₃-C₇ monocyclic cycloalkyl, C₄-C₇ monocyclic cycloalkenyl, or 4-7 membered heterocycle; wherein each G^{A} is optionally substituted with 1, 2, or 3 R^{u} groups;
R⁵ is independently hydrogen, halogen, G³, C₁-C₆ alkyl, C₂-C₆ alkenyl, or C₂-C₆ alkynyl; wherein the C₁-C₆ alkyl, C₂-C₆ alkenyl, and C₂-C₆ alkynyl are each optionally substituted with one G³;
G³, at each occurrence, is independently C₆-C₁₀ aryl, 5-11 membered heteroaryl, C₃-C₁₁ cycloalkyl, cycloalkenyl, oxetanyl, or 2-oxaspiro[3.3]heptanyl; wherein each G³ is optionally substituted with 1, 2, or 3 R^{v} groups;
A⁷ is N or CR⁷;
A⁸ is N or CR⁸;
A¹⁵ is N or CR¹⁵;
R⁷, R¹² and R¹⁶ are each independently hydrogen, halogen, C₁-C₄ alkyl, C₁-C₄ haloalkyl, —CN, —OR^{7a}, —SR^{7a}, or —N(R^{7b})(R^{7c});
R⁸, R¹³, R¹⁴, and R¹⁵, are each independently hydrogen, halogen, C₁-C₄ alkyl, C₁-C₄ haloalkyl, —CN, —OR^{8a}, —SR^{8a}, —N(R^{8b})(R^{8c}), or C₃-C₄ monocyclic cycloalkyl; wherein the C₃-C₄ monocyclic cycloalkyl is optionally substituted with one or two substituents independently selected from the group consisting of halogen, C₁-C₃ alkyl, and C₁-C₃ haloalkyl; or
R⁸ and R¹³ are each independently hydrogen, halogen, C₁-C₄ alkyl, C₁-C₄ haloalkyl, —CN, —OR^{8a}, —SR^{8a}, —N(R^{8b})(R^{8c}), or C₃-C₄ monocyclic cycloalkyl; wherein the C₃-C₄ monocyclic cycloalkyl is optionally substituted with one or two substituents independently selected from the group consisting of halogen, C₁-C₃ alkyl, and C₁-C₃ haloalkyl; and
R¹⁴ and R¹⁵, together with the carbon atoms to which they are attached, form a monocyclic ring selected from the group consisting of benzene, cyclobutane, cyclopentane, and pyridine; wherein the monocyclic ring is optionally substituted with 1, 2, or 3 substituents independently selected from the group consisting of halogen, C₁-C₄ alkyl, C₁-C₄ haloalkyl, —CN, —OR^{8a}, —SR^{8a}, and —N(R^{8b})(R^{8c});
R⁹ is —OH, —O—C₁-C₄ alkyl, —O—CH₂—OC(O)(C₁-C₆ alkyl), —NHOH, 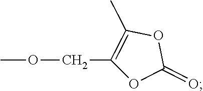
or —N(H)S(O)₂—(C₁-C₆ alkyl);
R^{10A} and R^{10B}, are each independently hydrogen, C₁-C₃ alkyl, or C₁-C₃ haloalkyl; or R^{10A} and R^{10B}, together with the carbon atom to which they are attached, form a cyclopropyl; wherein the cyclopropyl is optionally substituted with one or two substituents independently selected from the group consisting of halogen and CH₃;
W is —CH═CH—, C₁-C₄ alkyl, —O—CHF—, -L¹-CH₂—, or —CH₂-L¹-; wherein L¹ at each occurrence, is independently O, S, S(O), S(O)₂, S(O)₂N(H), N(H), or N(C₁-C₃ alkyl);
R¹¹ is a C₆-C₁₀ aryl or a 5-11 membered heteroaryl; wherein each R¹¹ is optionally substituted with 1, 2, or 3 independently selected R^{w} groups;
R^{w}, at each occurrence, is independently C₁-C₆ alkyl, C₂-C₆ alkenyl, C₂-C₆ alkynyl, halogen, C₁-C₆ haloalkyl, —CN, NO₂, —OR^{11a}, —SR^{11b}, —S(O)₂R^{11b}, —S(O)₂N(R^{11c})₂, —C(O)R^{11a}, —C(O)N(R^{11c})₂, —N(R^{11c})₂, —N(R^{11c})C(O)R^{11b}, —N(R^{11c})S(O)₂R^{11b}, —N(R^{11c})C(O)O(R^{11b}), —N(R^{11c})C(O)N(R^{11c})₂, G⁴, —(C₁-C₆ alkylenyl)-OR^{11a}, —(C₁-C₆alkylenyl)-OC(O)N(R^{11c})₂, —(C₁-C₆ alkylenyl)-SR^{11a}, —(C₁-C₆ alkylenyl)-S(O)₂R^{11b}, —(C₁-C₆ alkylenyl)-S(O)₂N(R^{11c})₂, —(C₁-C₆ alkylenyl)-C(O)R^{11a}, —(C₁-C₆ alkylenyl)-C(O)N(R^{11c})₂, —(C₁-C₆ alkylenyl)-N(R^{11c})₂, —(C₁-C₆ alkylenyl)-N(R^{11c})C(O)R^{11b}, —(C₁-C₆ alkylenyl)-N(R^{11c})S(O)₂R^{11b}, —(C₁-C₆alkylenyl)-N(R^{11c})C(O)O(R^{11b}), —(C₁-C₆ alkylenyl)-N(R^{11c})C(O)N(R^{11c})₂, —(C₁-C₆ alkylenyl)-CN, or —(C₁-C₆alkylenyl)-G⁴;
R^{11a} and R^{11c}, at each occurrence, are each independently hydrogen, C₁-C₆ alkyl, C₂-C₆ alkenyl, C₁-C₆ haloalkyl, G⁴, —(C₂-C₆ alkylenyl)-OR^{11d}, —(C₂-C₆ alkylenyl)-N(R^{11e})₂, or —(C₂-C₆ alkylenyl)-G⁴;
R^{11b}, at each occurrence, is independently C₁-C₆ alkyl, C₂-C₆ alkenyl, C₁-C₆ haloalkyl, G⁴, —(C₂-C₆alkylenyl)-OR^{11d}, —(C₂-C₆alkylenyl)-N(R^{11e})₂, or —(C₂-C₆ alkylenyl)-G⁴;
G⁴, at each occurrence, is independently phenyl, monocyclic heteroaryl, C₃-C₁₁ cycloalkyl, C₄-C₁₁ cycloalkenyl, oxetanyl, tetrahydrofuranyl, tetrahydropyranyl, morpholinyl, 2,6-dioxa-9-azaspiro[4.5]decanyl, 2-oxa-5-azabicyclo[2.2.1]heptanyl, 3-oxa-8-azabicyclo[3.2.1]octanyl, piperidinyl, azetidinyl, dihydropyranyl, tetrahydropyridinyl, dihydropyrrolyl, or pyrrolidinyl; wherein each G⁴ is optionally substituted with 1 —OR^{m} and 0, 1, 2, 3, or 4 substituents independently selected from the group consisting of G⁵, R^{y}, —(C₁-C₆ alkylenyl)-G⁵, and -L²-(C₁-C₆alkylenyl)ₛ-G⁵;
L² is O, C(O), N(H), N(C₁-C₆ alkyl), NHC(O), C(O)O, S, S(O), or S(O)₂;
s is 0 or 1;
G⁵, at each occurrence, is independently phenyl, monocyclic heteroaryl, C₃-C₇ monocyclic cycloalkyl, C₄-C₇ monocyclic cycloalkenyl, or piperazine; wherein each G⁵ is optionally substituted with 1 independently selected —OR^{m} or R^{z} group;
R^{s}, R^{t}, R^{u}, R^{v}, R^{y}, and R^{z}, at each occurrence, are each independently C₁-C₆ alkyl, C₂-C₆ alkenyl, C₂-C₆ alkynyl, halogen, C₁-C₆ haloalkyl, —CN, oxo, NO₂, P(O)(R^{k})₂, —OC(O)R^{k}, —OC(O)N(R^{j})₂, —SR^{j}, —S(O)₂R^{k}, —S(O)₂N(R^{j})₂, —C(O)R^{j}, —C(O)N(R^{j})₂, —N(R^{j})₂, —N(R^{j})C(O)R^{k}, —N(R^{j})S(O)₂R^{k}, —N(R^{j})C(O)O(R^{k}), —N(R^{j})C(O)N(R^{j})₂, —(C₁-C₆ alkylenyl)-OR^{j}, —(C₁-C₆ alkylenyl)-OC(O)N(R^{j})₂, —(C₁-C₆alkylenyl)-SR^{j}, —(C₁-C₆alkylenyl)-S(O)₂R^{k}, —(C₁-C₆ alkylenyl)-S(O)₂N(R^{j})₂, —(C₁-C₆ alkylenyl)-C(O)R^{j}, —(C₁-C₆ alkylenyl)-C(O)N(R^{j})₂, —(C₁-C₆ alkylenyl)-N(R^{j})₂, —(C₁-C₆alkylenyl)-N(R^{j})C(O)R^{k}, —(C₁-C₆ alkylenyl)-N(R^{j})S(O)₂R^{k}, —(C₁-C₆ alkylenyl)-N(R^{j})C(O)O(R^{k}), —(C₁-C₆ alkylenyl)-N(R^{j})C(O)N(R^{j})₂, or —(C₁-C₆ alkylenyl)-CN;
R^{m} is hydrogen, C₁-C₆ alkyl, C₁-C₆ haloalkyl, —(C₂-C₆alkylenyl)-OR^{j}, or —(C₂-C₆ alkylenyl)-N(R^{j})₂;
R^{yh}, R^{yi}, R^{yk}, R^{7a}, R^{7b}, R^{7c}, R^{8a}, R^{8b}, R^{8c}, R^{11d}, R^{11e}, and R^{j}, at each occurrence, are each independently hydrogen, C₁-C₆ alkyl, or C₁-C₆ haloalkyl; and
R^{k}, at each occurrence, is independently C₁-C₆ alkyl or C₁-C₆ haloalkyl.

2. The compound of claim 1, or a pharmaceutically acceptable salt thereof, wherein R^{A} is hydrogen.

3. The compound of claim 1, or a pharmaceutically acceptable salt thereof, wherein R⁹ is —OH.

4. The compound of claim 1, or a pharmaceutically acceptable salt thereof, wherein R^{10A} and R^{10B}, are each independently hydrogen.

5. The compound of claim 1, or a pharmaceutically acceptable salt thereof, wherein R⁷, R¹² and R¹⁶ are each independently hydrogen.

6. The compound of claim 1, or a pharmaceutically acceptable salt thereof, wherein X is O.

7. The compound of claim 1, or a pharmaceutically acceptable salt thereof, wherein
R^{A} is hydrogen;
X is O;
R⁹ is —OH;
R^{10A} and R^{10B}, are each independently hydrogen; and
R⁷, R¹² and R¹⁶ are each independently hydrogen.

8. The compound of claim 7, or a pharmaceutically acceptable salt thereof, wherein
A² is CH;
A³ is N;
A⁴ is CH; and
A⁶ is C.

9. The compound of claim 7, or a pharmaceutically acceptable salt thereof, wherein
A² is N;
A³ is C;
A⁴ is O; and
A⁶ is C.

10. The compound of claim 7 or a pharmaceutically acceptable salt thereof, wherein
A² is N;
A³ is C;
A⁴ is S; and
A⁶ is C.

11. The compound of claim 10, or a pharmaceutically acceptable salt thereof, wherein
Y is (CH₂)ₘ; wherein 1 CH₂ group is independently replaced by N(R^{ya}); and
m is 3.

12. The compound of claim 10 or a pharmaceutically acceptable salt thereof, wherein
Y is (CH₂)ₘ; wherein 2 CH₂ groups are each independently replaced by O and 1 CH₂ group is replaced by C(R^{ya})(R^{yb}); and
m is 4.

13. The compound of claim 11, or a pharmaceutically acceptable salt thereof, wherein G¹ is piperazinyl substituted with 1 R^{s}.

14. The compound of claim 12, or a pharmaceutically acceptable salt thereof, wherein G¹ is piperazinyl substituted with 1 R^{s}.

15. The compound of claim 13, or a pharmaceutically acceptable salt thereof, wherein
W is -L¹-CH₂—; and
L¹ is independently O.

16. The compound of claim 14, or a pharmaceutically acceptable salt thereof, wherein
W is -L¹-CH₂—; and
L¹ is independently O.

17. The compound of claim 16, or a pharmaceutically acceptable salt thereof, wherein
W is —O—CH₂—, and
R¹¹ is pyrimidinyl, optionally substituted with 1, 2, or 3 independently selected R^{w} groups.

18. The compound of claim 1, or a pharmaceutically acceptable salt thereof, wherein the compound is selected from the group consisting of Example 1-Example 151 of Table 1.

19. A pharmaceutical composition comprising a therapeutically effective amount of a compound of Formula (I) according to claim 1, or a pharmaceutically acceptable salt thereof, in combination with a pharmaceutically acceptable carrier.

20. A method for treating multiple myeloma in a subject comprising administering a therapeutically effective amount of a compound of Formula (I) according to claim 1, or a pharmaceutically acceptable salt thereof, to a subject in need thereof.
